SEQUENCE LISTING (60 sequences)

SEQ ID NO: 1 - DNA, 18 bp, synthetic construct
  misc_feature 1..18: note = Antisense oligonucleotide sequence
  modified_base 18: mod_base = OTHER; note = 5-methylcytosine
  source 1..18: mol_type = other DNA
aggcattataggtttttc

SEQ ID NO: 2 - DNA, 19 bp, synthetic construct
  misc_feature 1..19: note = Antisense oligonucleotide sequence
  modified_base 19: mod_base = OTHER; note = 5-methylcytosine
  source 1..19: mol_type = other DNA
aaaagtttctcagataggc

SEQ ID NO: 3 - DNA, 19 bp, synthetic construct
  misc_feature 1..19: note = Antisense oligonucleotide sequence
  source 1..19: mol_type = other DNA
ggacatgttaattttactt

SEQ ID NO: 4 - DNA, 19 bp, synthetic construct
  misc_feature 1..19: note = Antisense oligonucleotide sequence
  modified_base 17: mod_base = OTHER; note = 5-methylcytosine
  source 1..19: mol_type = other DNA
tgaacttgttatatacctg

SEQ ID NO: 5 - DNA, 17 bp, synthetic construct
  misc_feature 1..17: note = Antisense oligonucleotide sequence
  modified_base 16: mod_base = OTHER; note = 5-methylcytosine
  source 1..17: mol_type = other DNA
tagagttttgtgtgtct

SEQ ID NO: 6 - DNA, 17 bp, synthetic construct
  misc_feature 1..17: note = Antisense oligonucleotide sequence
  modified_base 2: mod_base = OTHER; note = 5-methylcytosine
  modified_base 16: mod_base = OTHER; note = 5-methylcytosine
  source 1..17: mol_type = other DNA
gcattataggtttttct

SEQ ID NO: 7 - DNA, 17 bp, synthetic construct
  misc_feature 1..17: note = Antisense oligonucleotide sequence
  modified_base 17: mod_base = OTHER; note = 5-methylcytosine
  source 1..17: mol_type = other DNA
ggcattataggtttttc

SEQ ID NO: 8 - DNA, 17 bp, synthetic construct
  misc_feature 1..17: note = Antisense oligonucleotide sequence
  modified_base 3: mod_base = OTHER; note = 5-methylcytosine
  modified_base 17: mod_base = OTHER; note = 5-methylcytosine
  source 1..17: mol_type = other DNA
ggcattataggtttttc

SEQ ID NO: 9 - DNA, 18 bp, synthetic construct
  misc_feature 1..18: note = Antisense oligonucleotide sequence
  modified_base 7: mod_base = OTHER; note = t is a uracil nucleoside
  modified_base 18: mod_base = OTHER; note = 5-methylcytosine
  source 1..18: mol_type = other DNA
tggacatgttaattttac

SEQ ID NO: 10 - DNA, 19 bp, synthetic construct
  misc_feature 1..19: note = Antisense oligonucleotide sequence
  modified_base 8: mod_base = OTHER; note = t is a uracil nucleoside
  source 1..19: mol_type = other DNA
ggacatgttaattttactt

SEQ ID NO: 11 - DNA, 20 bp, synthetic construct
  misc_feature 1..20: note = Antisense oligonucleotide sequence
  source 1..20: mol_type = other DNA
gttttaagattgtggtgttt

SEQ ID NO: 12 - DNA, 17 bp, synthetic construct
  misc_feature 1..17: note = Antisense oligonucleotide sequence
  modified_base 9: mod_base = OTHER; note = t is a uracil nucleoside
  modified_base 16: mod_base = OTHER; note = 5-methylcytosine
  source 1..17: mol_type = other DNA
tagagttttgtgtgtct

SEQ ID NO: 13 - DNA, 17 bp, synthetic construct
  misc_feature 1..17: note = Antisense oligonucleotide sequence
  modified_base 13: mod_base = OTHER; note = t is a uracil nucleoside
  modified_base 16: mod_base = OTHER; note = 5-methylcytosine
  source 1..17: mol_type = other DNA
tagagttttgtgtgtct

SEQ ID NO: 14 - DNA, 17 bp, synthetic construct
  misc_feature 1..17: note = Antisense oligonucleotide sequence
  modified_base 6: mod_base = OTHER; note = 7-deaza-8-azaguanine
  modified_base 17: mod_base = OTHER; note = 5-methylcytosine
  source 1..17: mol_type = other DNA
atagagttttgtgtgtc

SEQ ID NO: 15 - DNA, 19 bp, synthetic construct
  misc_feature 1..19: note = Antisense oligonucleotide sequence
  modified_base 5: mod_base = OTHER; note = 7-deaza-8-azaguanine
  modified_base 19: mod_base = OTHER; note = 5-methylcytosine
  source 1..19: mol_type = other DNA
aaaagtttctcagataggc

SEQ ID NO: 16 - DNA, 19 bp, synthetic construct
  misc_feature 1..19: note = Antisense oligonucleotide sequence
  modified_base 13: mod_base = OTHER; note = 7-deaza-8-azaguanine
  modified_base 19: mod_base = OTHER; note = 5-methylcytosine
  source 1..19: mol_type = other DNA
aaaagtttctcagataggc

SEQ ID NO: 17 - DNA, 19 bp, synthetic construct
  misc_feature 1..19: note = Antisense oligonucleotide sequence
  modified_base 8: mod_base = OTHER; note = t is a uracil nucleoside
  modified_base 19: mod_base = OTHER; note = 5-methylcytosine
  source 1..19: mol_type = other DNA
aaaagtttctcagataggc

SEQ ID NO: 18 - DNA, 18 bp, synthetic construct
  misc_feature 1..18: note = Antisense oligonucleotide sequence
  modified_base 13: mod_base = OTHER; note = t is a uracil nucleoside
  modified_base 18: mod_base = OTHER; note = 5-methylcytosine
  source 1..18: mol_type = other DNA
aggcattataggtttttc

SEQ ID NO: 19 - DNA, 18 bp, synthetic construct
  misc_feature 1..18: note = Antisense oligonucleotide sequence
  modified_base 6: mod_base = OTHER; note = t is a uracil nucleoside
  modified_base 18: mod_base = OTHER; note = 5-methylcytosine
  source 1..18: mol_type = other DNA
aggcattataggtttttc

SEQ ID NO: 20 - DNA, 18 bp, synthetic construct
  misc_feature 1..18: note = Antisense oligonucleotide sequence
  modified_base 2: mod_base = OTHER; note = 5-methylcytosine
  modified_base 18: mod_base = OTHER; note = 5-methylcytosine
  source 1..18: mol_type = other DNA
gcattataggtttttctc

SEQ ID NO: 21 - DNA, 18 bp, synthetic construct
  misc_feature 1..18: note = Antisense oligonucleotide sequence
  modified_base 2: mod_base = OTHER; note = 5-methylcytosine
  modified_base 15: mod_base = OTHER; note = t is a uracil nucleoside
  modified_base 18: mod_base = OTHER; note = 5-methylcytosine
  source 1..18: mol_type = other DNA
gcattataggtttttctc

SEQ ID NO: 22 - DNA, 18 bp, synthetic construct
  misc_feature 1..18: note = Antisense oligonucleotide sequence
  modified_base 2: mod_base = OTHER; note = 5-methylcytosine
  modified_base 14: mod_base = OTHER; note = t is a uracil nucleoside
  modified_base 18: mod_base = OTHER; note = 5-methylcytosine
  source 1..18: mol_type = other DNA
gcattataggtttttctc

SEQ ID NO: 23 - DNA, 18 bp, synthetic construct
  misc_feature 1..18: note = Antisense oligonucleotide sequence
  modified_base 2: mod_base = OTHER; note = 5-methylcytosine
  modified_base 13: mod_base = OTHER; note = t is a uracil nucleoside
  modified_base 18: mod_base = OTHER; note = 5-methylcytosine
  source 1..18: mol_type = other DNA
gcattataggtttttctc

SEQ ID NO: 24 - DNA, 17 bp, synthetic construct
  misc_feature 1..17: note = Antisense oligonucleotide sequence
  modified_base 2: mod_base = OTHER; note = 5-methylcytosine
  modified_base 13: mod_base = OTHER; note = t is a uracil nucleoside
  modified_base 16: mod_base = OTHER; note = 5-methylcytosine
  source 1..17: mol_type = other DNA
gcattataggtttttct

SEQ ID NO: 25 - DNA, 17 bp, synthetic construct
  misc_feature 1..17: note = Antisense oligonucleotide sequence
  modified_base 2: mod_base = OTHER; note = 5-methylcytosine
  modified_base 12: mod_base = OTHER; note = t is a uracil nucleoside
  modified_base 16: mod_base = OTHER; note = 5-methylcytosine
  source 1..17: mol_type = other DNA
gcattataggtttttct

SEQ ID NO: 26 - DNA, 18 bp, synthetic construct
  misc_feature 1..18: note = Antisense oligonucleotide sequence
  modified_base 2: mod_base = OTHER; note = 5-methylcytosine
  modified_base 11: mod_base = OTHER; note = t is a uracil nucleoside
  modified_base 18: mod_base = OTHER; note = 5-methylcytosine
  source 1..18: mol_type = other DNA
gcattataggtttttctc

SEQ ID NO: 27 - DNA, 17 bp, synthetic construct
  misc_feature 1..17: note = Antisense oligonucleotide sequence
  modified_base 2: mod_base = OTHER; note = 5-methylcytosine
  modified_base 11: mod_base = OTHER; note = t is a uracil nucleoside
  modified_base 16: mod_base = OTHER; note = 5-methylcytosine
  source 1..17: mol_type = other DNA
gcattataggtttttct

SEQ ID NO: 28 - DNA, 17 bp, synthetic construct
  misc_feature 1..17: note = Antisense oligonucleotide sequence
  modified_base 2: mod_base = OTHER; note = 5-methylcytosine
  modified_base 7: mod_base = OTHER; note = t is a uracil nucleoside
  modified_base 16: mod_base = OTHER; note = 5-methylcytosine
  source 1..17: mol_type = other DNA
gcattataggtttttct

SEQ ID NO: 29 - DNA, 17 bp, synthetic construct
  misc_feature 1..17: note = Antisense oligonucleotide sequence
  modified_base 2: mod_base = OTHER; note = 5-methylcytosine
  modified_base 5: mod_base = OTHER; note = t is a uracil nucleoside
  modified_base 16: mod_base = OTHER; note = 5-methylcytosine
  source 1..17: mol_type = other DNA
gcattataggtttttct

SEQ ID NO: 30 - DNA, 17 bp, synthetic construct
  misc_feature 1..17: note = Antisense oligonucleotide sequence
  modified_base 2: mod_base = OTHER; note = 5-methylcytosine
  modified_base 4: mod_base = OTHER; note = t is a uracil nucleoside
  modified_base 16: mod_base = OTHER; note = 5-methylcytosine
  source 1..17: mol_type = other DNA
gcattataggtttttct

SEQ ID NO: 31 - RNA, 18 nt, synthetic construct
  misc_feature 1..18: note = Target sequence A1CF 6953-6970
  source 1..18: mol_type = other RNA
gaaaaacctataatgcct

SEQ ID NO: 32 - RNA, 19 nt, synthetic construct
  misc_feature 1..19: note = Target sequence A1CF 2181-2199
  source 1..19: mol_type = other RNA
gcctatctgagaaactttt

SEQ ID NO: 33 - RNA, 19 nt, synthetic construct
  misc_feature 1..19: note = Target sequence A1CF 16970-16988
  source 1..19: mol_type = other RNA
aagtaaaattaacatgtcc

SEQ ID NO: 34 - RNA, 19 nt, synthetic construct
  misc_feature 1..19: note = Target sequence A1CF 38053-38071
  source 1..19: mol_type = other RNA
caggtatataacaagttca

SEQ ID NO: 35 - RNA, 17 nt, synthetic construct
  misc_feature 1..17: note = Target sequence A1CF 78973-78989
  source 1..17: mol_type = other RNA
agacacacaaaactcta

SEQ ID NO: 36 - RNA, 17 nt, synthetic construct
  misc_feature 1..17: note = Target sequence A1CF 6952-6968
  source 1..17: mol_type = other RNA
agaaaaacctataatgc

SEQ ID NO: 37 - RNA, 17 nt, synthetic construct
  misc_feature 1..17: note = Target sequence A1CF 6953-6969
  source 1..17: mol_type = other RNA
gaaaaacctataatgcc

SEQ ID NO: 38 - RNA, 18 nt, synthetic construct
  misc_feature 1..18: note = Target sequence A1CF 16972-16989
  source 1..18: mol_type = other RNA
gtaaaattaacatgtcca

SEQ ID NO: 39 - RNA, 20 nt, synthetic construct
  misc_feature 1..20: note = Target sequence A1CF 26358-26377
  source 1..20: mol_type = other RNA
aaacaccacaatcttaaaac

SEQ ID NO: 40 - RNA, 17 nt, synthetic construct
  misc_feature 1..17: note = Target sequence A1CF 78974-78990
  source 1..17: mol_type = other RNA
gacacacaaaactctat

SEQ ID NO: 41 - RNA, 18 nt, synthetic construct
  misc_feature 1..18: note = Target sequence A1CF 6951-6968
  source 1..18: mol_type = other RNA
gagaaaaacctataatgc

SEQ ID NO: 42 - RNA, 20 nt, synthetic construct
  misc_feature 1..20: note = Target sequence A1CF 6951-6970
  source 1..20: mol_type = other RNA
gagaaaaacctataatgcct

SEQ ID NO: 43 - RNA, 20 nt, synthetic construct
  misc_feature 1..20: note = Target sequence A1CF 16970-16989
  source 1..20: mol_type = other RNA
aagtaaaattaacatgtcca

SEQ ID NO: 44 - RNA, 18 nt, synthetic construct
  misc_feature 1..18: note = Target sequence A1CF 78973-78990
  source 1..18: mol_type = other RNA
agacacacaaaactctat

SEQ ID NO: 45 - DNA, 86267 bp, Homo sapiens
  source 1..86267: mol_type = other DNA
tttgatatgacgattagagcataacccgagtgacacgttgaattcgccataatcaaggaaaccttttccgggtggggatctctgaaattactcaggtatgatccatgtgttttgcttttttctatttcaactctgtgaaactgtgtattgtggtatcagtgggaatgcagtgaaaacagcttttaccctcttctgttatcgagcctaattcaaagaattatctagcttgtttttagactaatgaaacaaccagaaaacaaaagaatatattggaattattatataatctctaaatgagatagctgccaggatttaataaatttctgtagacttaccacggagggttcaagagtggagccaatgttgtgagaagtttatttagctctaaccagacagcagggagagttgtggttgagtgcctgtcccaatcctaggaccctgagaaacaaaaatataattaacaaaagaaatagtattaacatcaccagcagattttgagttctgtgtgtgggtcataattcctatcatactttagacttcatatattcaattatctccagagctactaccaccataaaaaagaaagtcttggtacaactcttgcgattccgcctgccattatcttgctaaccaaaggaatgtttaaactattcacttttgattctgaccaaaaaaatattatggaaaatgactggttttcagtgtgttaaatctgaaccttggaatggctaattattatattgtttccacaatttgggcaactgtgctgaggtggatatattttcacatattttttttgtgtactccaaaaacattataatggtattttagctaaagaaagaaatcaatctggacatttaccaaaaatatcccatttttaccagaaaccccctaaactctctgactttttgtcatgattacctatgtaaattccaagaagaaaggattccaaactaaacaattttatttactataggacttttcagatatgtattgtatatctccaagaagatgaagaacacagcattatttgcacattgtgcaggattagtgttttatagatcgtattttggcaaattcagctctagttatagtttaggagcatcaaaaaatgtgttggtctgctctgaaattttctttataacgtgtgctatggagcatgcaatgggagcacaggtctggaaatgacagtgatatcacttatttgtcaaagcagtaaggttttgccctgctaaatacagagaaagagacaaatgacttataaggttcatactttctggaataacattgactctgcccatttcagacaactcttaacattttactgttctattttactatttatcgatttcaagggaaaaaagaagacagctggcaaggaaaagaaatattgttgtgggcaaacattataaaatgacataatggatattaaaacactataaatactttgttcttattttctactgcactaatgccaaggtggttttatgtcatttcaaatatcaacatttcctacactatattcagtggatgttaataaatgtggctcaaaagaaaagcaactccattgtcaagtaagttgaggaaacagtgttagaaaagttatagcaggttttgttaccacatgagtgttttgagtaataatgataacaagcatctgtgaagcactatattatcgggtctacttctaagttctctccatgcattatctcatgttattcttggaacaactctatggtgtgatactattataacagtttataaatgggaaaatggaggcacagacaaattacctaacaggctcagctactcagttagcaagtggcagagccaggctcctttgtctggtagtctgatccagagcccaagcacgtaaccattatatcatacgctcgagcactggggactcatgacacatgcaaatattgtcatttatcacaactatctaagagagtaggcaacgttttgtttcctgggcttaatttacaacagaacatcttctttcaggaacatctatcaatatctctcaaaattggcatttctcagaatacagaacaagaaagaccaaatcgcttttatagcccatgtccagcattcagaacaatcagaatccattgaaataatcacagttgaaaaatctaaaggactttagagtctagtcttccaccaaataggactatgcctatctgagaaacttttaaaattaattacggaaagaacactaggctttgtgcctcgacacagctaagtggaaacgcctattgtaaaactccctaggtttgtgaccttgggcatattgtttgactccactgaatctcagattcctcaaatgtgaaatgggggtaagatctgacccatagtattttgtgaagtttaaattagatgataaagcaaaggagagaaatgtggtccccggtacttaagaattcagcaaaaaccccacttcccttttcattttcctatttgtaacaacctccagaaaaattttctaaaattgcccaagtatacctattaggccttgtttttcaaaacaatactaataaagttgaaatcacaaactgtacataaaaaattttgattaatcacaaatagtagcactattccatttattcagagaaagacagtctgtaaatttttttagtattaaagcacacacacagagtatatttttattttagagattattgctgaagaaaactcttagttataaaaccccaacaccttttcacccaaacccatgggacaccctgacacagtgcatgctcatttcatcatggaggcagttgatgttaaaaatctgtttcagtaactgtcttttaaaaattgtcatcccttagaaagctgagtagtactgtgggtactattgcacaatggttttcattaccttatgtgtctctaaaatgactcaataagttcaaacaagacagtattgatcgacagaggataaacaccattttatcttattcacagaaacagtaaccaggctcctgcaaagtgcagctctcacaatccaactgcccccaggaaaagcatctggaaaaggagcagcactcttgcggtgacattctgtattagaaatctgtatcccatctgctccttggcctgatcctgtaaaatctggagagttattaacaaatgtttgctgctgattcaaccaggaaagctagttcatcatttcatttcatcaaatctactccaactaggttatcagttttttgttgttgatgatggtttgtttgtatgagatggagtatcgttctgttgcctaggctggagtggtggtgcaatctcagctcatcacaacctccacctcctggattcaagtgattcttgtgcctcagcctcccgagtagctgggattacaggcacacgccaccatgccaggctaacttttgtatttttagtagagacggggtttcattatgttggctaggctggtctcgaactcctgaactcatgatctgcctgcctcagcctcccaaagtgctgggattacaggcatgagccaacatgcccggtctaggttatcagttttaatgaccacttagaatttttaatttaataaactaaaattttgtattgaatatttttgattgatgcaatccaaaaattaaaacatgcccttttgtaaaaaatcatcgtcctgtataagatatgctggtatcttgagagctctaatttgatttcttttaaacatatgtaagctaagatttttatcctatatagatttatatgtggagttttatgcatatatgtattaacaatcacatggagaacattttccaagtttgtttattcaataggaattggtccctggtaatattaagagagaaatactaataaggatgtaagataaatccttctgatttgcacagttaagtgcaatgctctgctccagatgtaatatgactaaagagaattaacacatgacaatttaggcagacagaatgtctaagtgagcagattggtttataattctataaaatcccacctgcagtagtgatgtagtgatgccttccctccctccagcaattaaaaactattcattgttttaggatgcccaattgatttaaaaatcactaggtttaagtagtagaactaactacaatatcttatttccataggatattctagtttataaagcttttcatatacaagaacttataaagatttttaatggcaaatttcttatagttgatgtcaattcagtatgcctagtttcttcaaggtaactgtgtcaatattcaccattccaattgtacaaatctgattaactgaaagatgctcccacctatcgtttataagcccaactctgtttatgtaacacaatgatgtccaacttcactggacattaactaatgtacctatcagttaagaggctagatgaggccgggcgcagtggctcacgcctgtaattcgaacactatgggaggccaaggcaggcagatcacctgaggtcaggagttcaagaccagtctggctaacatgacgaaaccccacctccagaaaaatacaaaaattagccaggcatggtggtgggtacctgtaatcccagctacccaggaggctgaggcgggaaaatcacttgaacccaggaggcgaaggttgcagtgagccgagatcgcaccattgcactccagcctaggcgacagagtgagactccacctcaaaaaaaaaaaaagagtctaaatgaaaacagtgaagagaggacataaaggtccatgtggaaggtgagagaagaaccacccccatatgggacctagaagaaaacagccctaaaaagatgtaattataactcagtccagatgctgtgactaacacagtgaaagatataaaggaacagaggtttataaaatctcctacctgcctggcaggggactggcaaaacattgtgcacaaatgaaattttttatttctattgattttagtattctatccagctcttctactgggattctgcttagtgcttatctttccatctggggccttcagcatgaactaaaatgtactttaaattattagatgtaattaaactctaatgcctctgattatttgaaatggaagtcagaaatcaaaatcaggttccatggggggaaaactgtgttggcacatatagatgttcatctcaggaatcctctttctaaccttcttttccgaagatccctaagagttcctcgccattcatgtacttactaccaactaattataattggcaccctacgatgatgataacaatgatgttaataaaatctcttacatcctcttacatcctgccacatgccaggcacataggttaactcatttaatcctccatcactctatggaatgggtactattgctacgccatttttccaataaagcagctacagcttagggacacagagaaatttgccccaggtcacaccactaataagtggtgcaggagtgagtccgggcagtctggcacaggccaccctgtgaacaaaggacaaatggaacactgggaagcatcaaggcttagaactgaagaaaacttttgagatcctttatttgacagagaaaagcaagttgggttccccagcttgggtttttgttagaatgagaacccaagagcagaacctgaaccctctgtggatacttttccattataccacttcccactgtaaagcatttctaattcagcctgattctctgtgaaaattctgagagacgaattaatagaatccctatagttaaacaggaatactcccttcactccaagtctcaccttccttgctgtgcccagactcatggactgacctagccagagactcagttaaaggagacccattttgtgacttggagagactgacaggcccacatcacagaggaaatacatgcagacacagacatggatatggtttagctttttgtccccacccaaatctcatcttgaattgtaatccttaggcctgaaggaaagaatctggtgagaagtgataggattatgggggtggtttcccccatgctgttttcatgataatgagtgaattctcatgagacctggtggttttataagtgtttgacttttccttctgtacacactacttctctcccctgctgtcacataagaaggcctaagcttgcttcccctttgccttccaccatgattgtgagtttcctgaggcctccccagccatgcagaactgtgagtcaattaaacctctttctttataaattacccagtctcaggtatttctttgtagcagtatgagaacagactaatacaggcaccatgaacatagtgacaggataaaccatggaggcattgataaaaataaaaagtaattgtgatgaatgacttggttgcaattacactcaaattctgttagtgtctgaagaggacatagcacctcctacctggaggacatacccttttccccatggaaaaaataattgggctgctaggtctgtgtctgctaagggagtcccatatttaaactgacctaattcagaactttaaattgagactcagatgctcccaaatagcagctccaccaacctgaatagagttactcatttagaataagagtttgttcccacaagttttgttaaaatacaaattagttgggatgcggggtgtgagtgatagggactaaaatcgcttaacagactagcctggcaaagccctgtgggaagcaagttttgatgatagggaagagaagatgtcaagagcagatggtccagggaggaagtgtcacaagtaacttgggggtgatataagggacttgagtagcaggtatttgtctgatctttaaggaattaacttattaaagaattttgttagtaaagtcatttgagtatcacacttgcaacatactaattcatccatgtctagttttctcattaaatcacagatatttaacaggtgcattaacttgaattgacatcatgcagcaaatgccattatatggacccttaagatattttaagagaaaaacctataatgcctggaaactcttagttttctctgacatttaaaaacaagccgtaaaatcaaatcctttctataataatttagtaactcagaattgcaaaggatttcctaaagcccaatgatccaaagctctgacctgtgatgttggcatagcagagtgggagacaaatcatatgttaaagcagaaggctctggatctggaatgcttggatacaaattctggctctaccacttgctacaatttaactctaggcaaattactgaactctgtgactatttcctcttctgtaaagtcagggttgtttcaataattagatcagaaattgtaaagtgcttagaataataccaagcaagtgtgcataaactttagccatttagctataactctcatcatatttctgtgttgattacaaaatgctttactagaggaaaatagatgagaaagaggccatgaggtaaaaatgtacagtgctttttaaagtagaatgaggtggcttagaaattaattctatccccatcactggcttgtagtagaaatttgaaacagtctctccctgattctctggcagtttttctctttttttcttttttttctttttgagatggagtctcactctgtcacccaggctggagtgtagtagcgtgatctcagctcactgcaacctccgcttcccgggttcaagcgattctcctgcctcagcctcctgagtagctgggactacaagcatgtgccaccacaactggctaattttttgtatttttggtagagacagggtttcaccttgttagccaggatggtctcgatctcctgacctcgtgatcctcctgcctcagcctcccaaagtggggggattacaggcgtgagccacggcgcccggccctctttcagttttttctaagacagagacactccctatcacagacagggtcttctaaattagtagagaatgatttgaaggaaactgggtaattatcagaggaagtaaaagagaaatacagatcctcacaccagaatgcaaatctctaagactttattattttcatcactatgaaagaaaagatcagtactcaaagccacttatgatgatgcatgtaaattagcataaaatcagaacaaatataatatggcttttaaaatagaaacataaggctatctttccttacaagtgtcagattgcttctgtgccactgtaacacagagctttagatggattttttaaagcttcactcagagatcaaatgtactccattagtcagtgattggcagacttggtgaaaattgagttttcaacttaaatttctccaaattggtctgaggaaaccagatgcagcaactaagttcagaaggtcagagtctcaataaggttaaaaagagagggagaaacataacactagaagtatgaatgaatgaaatagaccacacacagctcatcaggtctctataatataccacgcaaataattggtaccaggactattgaataatcaactgcttttaagctgaagaatatctacattgtataccatgattaccactttaccaattcaaaagacaacttaaaaattaaacatatatacaaagacattctgcttttttcattaggccatgtcattctataatttctaaattctgtcaactctcttccccaggaatcttctagaagtcaggcattttgggaatagttgagtatcataattgaataaagatagcacaacagtcacctgtagtagtctgatcttacaggtactagaacaataacagcacaaaaacagagttacgtgtatgtattattcagtgttctctagaagaaaacatagtatatgtatatataggcaatacatttataaatgcataaaacaaatataaatttgtgtaagtatgcatataaatatgtaagtctacacatataaatacaaataaagagatttattctatgggattggctcatgcaattatagaagcaggagtcccaagatttgcagtaagcaagctggagattcaggggagcccatgcatagttccagcccaagtccaaaggcttgagacccagaaaagcaatagtgtaagttccagtccagaagccatcaggttagagacgcaagaagagccaatatttcaagtccgaagatcaggaaaggccaatgtccctgctctgtcatgcaggcccagctccctcttactcagcttttatgttttattcaggttttcaattgattggatgaggcccactcacattgcatctgctgttaaaattttccaagatctttttggggttaaaaacctatcattcatctctcaatcttcagtgttgacaaggacagagcacttatgagtattcctgtttctgagtttctgataagcttgtccaagtggagcacaggctggacaatttataagacagtattgaagtcagagcaattacaaaaggaaaggggtaggtttcttgtgtgatgtgctgcaaacactgacctctcagaatttattcggccctgtgtgtatatacgtattcatgtctgcacatatgtgctagttcacttatacatctatacatacacacaatcacatatacccaagatctgatggaatagacatgaatttggcttagccagggaaattatagaaagaataatgggaatcctaggcatgtgggaaatgggaaacaaggacaaaggcgcctaggtaaaataaaaagcaactggtgtgcctaggagacctgatggacttgagcatggaatagctccagacttgtattgaaagaaaatcaggaaagtcaagttacgaccaggtcatggcaggcactgaatgtcaggttaaggggctgaactgtttctttctagttagtgatgggtccagtgaaggatttggggcaggagaatactagaaggaatattttagggagattgatcttccagtggtgtgcagaacagatatgccatgcctagagggtagtaagcagtgacatttctttcactctacactttagctgagctcagggtaagtccagtaatggaagatggggtgggaaatagagctaggagtgtagagagggcaacaggtaaggatacatatgtaccaaaagcagaaaacaagttaagatccaggcttcatgaggtgcctatattgaagattgctctggataaagagactgttaggacttcagagttagaattcatagcaattcacagaactgctcaggctcttcattattcacctgactcaactctcctacatctgctcctttctcctaagagatttccttgctctctgtcctttctctaccccacagcttttgcttcctttccgtacatagaaccacacaacaagtactagttttatgcatgcagtggtcatataaatatcttattggcgagtcctcatcaaaactgagttgaaacaaaccatttgagccagaatttagtaagagtcagattttttaaggtacttcaaggattttatttataaatacaactaggaaaacatttactcattagaaatgatgaaaccatagacaaaacaattattagagaaaattttgaattattcttttctcctgctgtctaatttctacctttctcatgtagtctttttctttttaattagattttttatgtgagtatatggtccatgtatttggctggtggaataaattcaattattctcaattgttttgtttgacctgtcacgtgtgtaaattagagcttcagtcagctgttctagatgcaactgcatattgttatcacctgagaagctttaaaatctgtcatttcctgggccccattccaaaccagctgaatcagaatccttccgggagggtaagatgatgcctaagcacaggtgattctaccatgagtccaggcagggagtctggtttttgtgcacagcttctgtatgtcatggctattggcacctggctctgctacggcggttcttaagtgatcttttaatgccgattgggagtatgttttaatagtggcacaatattttaagaataaggctttaaaggaagttattgcaagtaacagttaagggatttactcaaactggacacaacctctgttggagcagattacagtgaagttttgggggcatgggataaaaaagaggaaggagagatttaatggagcgaatattatttgctaaaatttaagaacgtttgcagttaaatctcgtactattgcaacaagatgtgcacaggtgcataggtgaatgcttcctcttctggtatttccttgtgtcatttatagttcaagttaaatcctagggatttaaagtgcttctcattgatatttaaagggcatttttaacaaatggagaaatgtctttcgaaacagataaaatgtacctttataaaatccactttatgagaatatgctaaaaaggaagatgtgtttctattggatcataaattatacaattttaaacagatttttatatgaaagtaaattgttccaactatttgaaattactttttcctcaaacaaaacaaaaaagatttctagatgccaggattgttttgcaaaaatgatgatctgatgatctgtgggccagatgaatatgcttaactttcctaagagttaaggtaacatttttcctttaatgagcctgtatctgatttctgatttgatgtagttttttaaaaattgagaaaatatgcctcctctaccccttgcatgttttaatgccctgatttaatgccggtgtgtgtacatatgtgcatatacacagatatatacatatatacacaaacacatattaaaattataataaaataatcatttgttcattttatacctgcttgttctttttctctgataaactctaagacagtggttcctatggtattttttctctgatatagtttttcttttctctatagatattttcattacagtgcaatttttccagtgtcttaggttttgctattttaatttactctacttaggacttagaatgattccatataggatggtatatagattttaaacttcaacaaacagtaatcaaacatcagctagccctacgcatccttgcaaaaataacaatttatttagaaagttgtatcagcattactcctctacaacatttaatatcttgccttaaaagttcaatatacatgtcagatggattcagaaacttctttagccatgggtgtcaccaaaagagcaggaaagggaacaaaaaattaaagaataaaacagaaaaagagaaaaaggaaaaatgaattatatggtcatttgaggatggagagtgagaagaccttactcattccctgtgtgtagctgtgtgtaacacaactacacatagttgtgttagctgtcttgagaagcacttggggctttgattacctccattgcaacctcatgagatatttggtctatagataatttcttcttctcttgctatctacaatccagaagaactaaagatcagtggaaagaagaatcaacactttaaagaatttaaatggctgggcgaggtggctcacacctataatcccagcattttgggaggccaaggtgagtgaatcactggaggtcaggagttcgagaccagcctggccaaaatggcaaaaccctgtctctactaaaaatacaaaaattagccaggcatagtggcacacatctgtaatcccagctacttgggaggctgaggcaggagaatttcttgaacccaggaggagaaagttgcagtgagctgagatcaccccaccacactccagcctgggtgatagagcgagattctgtctcaaaaaaaaaaaaaaaaaaaaaaaaaaaggaacttaaatataagaataaaagtaagcatgaaagctgagccagatcttgccaaatgatacatcatttggtaaaccatgaaggcgccacacttgccaaggagtaacatttcaatgagatatgactctgcatagcagacagctgggtccatttggaccatatccacaggggatcaatccttgatgtgtccgagtgaagcatcagacaatccacccacaccactcctcgaaggagctattacaatagctcactttcattccggcaaagaggctaagttaccatagtgcttcagatgctccagaccatgtgtccctatggaagagaatggggaccacagcatccccctcattggttcattatggtgtcctgtgttcttcacagcaacactttaaggcaggcagggtagatataatttccacaccccctgcctatttttttttaagatgaggaaactggaaagaagtaaaattagtaattaggaacacaatctcagcctccaggctacctaaattcaaatcatctgtctgccactcattaccagtgtaaccttgggcactttagcctctctctaccttgatttattacccatatcataggaataataatagctacatcactgagcttttatgaggattaaatgaattatgtttaaaattttcagaacatagcatatgtgttcagtaaatgttagctatgtttattatccttatttaaaatatttttacatattgccacataataggcaaaaaaatgttatcttggttttttgttttaatttgccttttcctgatcactaaagattaccatttttaaatcatgtttataagctattttgatttctttaaattgacagttcatttcctttgctcatttgtccactgatatgtgtctattatttaaaagtaatctgtatattacagataatatttctctatctattatataaataacaattattctcttcctgtctgtagactctttttaacttcatttattttgccattacagaagtttgtaatatttttgtagtcaaatctgctagtcttttcatttatggttcttaatatgcttggtttagaagaccttctgtaccatgagattataaaaacactctactatatttacttctggaagttacacaatgtttttaaaggttatttctttattccatttagaatgctggcatgttatgaggtgatatgcccactttattttttttctcaaattgtttatgatccccctatgaatttgaaatagcacctattggtgaattccagtataaacaaaactatgggtccagagtttttattttactccattaatctggttctctatttctatgcttgttttaatctaaactgttaattagggctgttccaaatcattatacactttcaatatatgttgtggcatttattgcacattgttcctgcccggtgaactatgaaatcaatttgtcaaatttcagaataatttcattggactttaaattagaattgcattaaagtaataatttgggaaaaactgatatttttacatcagcaaaacttccatttactaaggcctttagatctttcagtcaaattttatcatttttttatataggttttaaacttttctcgttaagtttattcaacttttatgtttttattctattgtttagaccttattatacttcacttgttatttctattaaatacagaacatttattttataattttccttgtagtaatctttcattagtttcaatcatattatatagttgatttacttgtttttaatgagaataaatgattgtggtgtttcattactaagtataatgtttgtttcatgtttctgagagatgctctttatcaatttaaggatgttttgttctattgttcacttatgaagattttttttaaaccaatactgggtattaactttcattaagtgccatttttagatttactgtgatgatcacgtgttttctttttaacagtgaaatgaattaaacttcgataatccctaatgtttaataaactttatatttttgtagtgatcccatcttgttcattgtatattattattttggatgctttttcatatgtagttcatatctatactcctatgatactggtctagtgttctactttgtagtatatttttctctatcagatatgatgagtttgtagataatttcagaagttttctattattttctactcttcaaaaggctttacatggcaaaggaataatttgcttttaaagagatttataaaacttgcctaataaaaccatctggatctggtgttatttgggaggaaaacattgaatgttttcaatatagattatgattactagtttattcaaatttttaacttatccttgagattaaaaattcacacttttctaaggttttgtccactacatgtatatttttcaaattattggtaaaagtattctcttttttaaaagtctatgaatctttggttatatccaatttttctcttgttttgaaaatttattttctcctttttaccttgtcaggattgccagctgaatgctaatttactactacactcagctgttacatacatttatcaagtctacaaatttgttttattaattctacttttatctttgattccttccaatttctttttgtccatttcattcttttcctagcttctttagttgaatgcttattttcagtgtttattgtcttgtaattaataataaacagactatcgtttacaaaaatctagtgtacatttcaaaatagctagagaaaatgattttaatgtttttagcataaagaagagacaaatgtttaaagtgatagatatcccaattatacttctttaagctttacaaattatataaatggattaaattatcacatgtaccctcaaaatatgttaaatctattatgtattaattaaaaaagaaaaacaagaatagaaacttgaccacatcttcatgaaaagtatgatattttcattaacatccatttattaacataattttaacttttatattttatattatgagcaagagctatatatcataggaagctaaaaatagtcaaataagcaagaatgaagaaaacaaaaataatttgttaatccactcacccagagataacaaattagccttgtgtaatcaaagcatatttgaatataaaaattaatacaaatgaaatcagaccatacattctgttttgtaacctgccttttctactttattatatttcacaagtatctgtctaaattattatatattcttctagaacattgatttgattagctgtactgtatcactgcttattcaaatatttctttatcattagatatttcagttgtgcctacctttcataacaatactaatatattctatgagaggtgacaaacatgaacagtcttaggaccttaacaacttttctactaggtagcaaaaacaaagataaatgaaataactggatccattagtaaggcaatataaaagagctcaagataatgcattcgaaattgaatttttctctgcattggagaaacaggaggagaataaccaatgagaaaatcacaggtcatggctaagatgagttaaacaaagcagcaaataaagaataagaatagaatatttctacagcaccttctatatgccaggtactatgttaggcgctctcacattcattaccttatttattccatgcttaaaagaaccttcaagttaggtatcagtattcttatccttttttagactagaaaactagacctcagaaaagttaaaatcatggtggcagaactaaattttttttaacttttttttttaagttcagggatacaagtgcagatttattacacaggtgaacttgtgtcatgggggtttgttgtgcagattatttcatcacccaggtgttaagcctagtacccactagttatttttcctgttcctctccttcctcccaccctgcatcctctgaaaggtgccagtgtgtgttgtccccctctatgtgtccatgtgttcccatcatttagctcccacttatatgtgagaacatatggtatttggttttctgttatcctcctctgcagcaggttccacaattttaaagatgcttacatttacatttcagttctgtccctgtgtaagtttgctacagttaattcaagaacagaactgaaatttcagtgtaagcttgttcttctttaaaaatgtggaaacctactacagaggaaaatttttgtcaatacttacaagtaaaattaacatgtccactatgaacaacttttacatttcttttctttctttaatttaagccttgtcttggcttaaattgacaagttccaaattaatatatgattttttgctaatgcctgaaaaacatagaggaggttctggttagttttttttctagtattgttttaaaaaactaagatgttgcacattgggactttttgtgatgtcttcatggctatattttgaaattttatgtatacatattctctattaattttgctatggctcatgatccatgaccaaagtgcatgctaccacaagaattaatttccaaaatagacttaagatcaattgtaaatatttttccaaggtggatattagagctttgtatcaagcaatgcaaagcaatatttacttagcatcatcaggcaaatatatagacttcagtcataaatccttcaccaaccatttcagtactatctagaatttctttcacgtaaaattcccaggagatactcaagggctgaaattcgtgctaaaaccacttgtacaacttgcatgttacataaaagttatgtccagcctacatcatgtaatgtcaaggcacaactaaacattgaggaaagactaggttcagtttggagaggtcccagaggcaagtgaagccatcatggatttgaaatgcacgtggagcacctcactgtacgggccccagtggcttctcattcaattatgaaagaacacttctctgcttaccaagtcagaaatagtctttgtccccattcagtttccttatccaatttcttccgaaaaatcaaatcaagatagtaaacatactattaacagaatgccttcatttcctaactctaaattgtctaccacgttgcagatattaaattaatatttgctgatccttttgttaaagtcgaggataattactttaatttttaaccaagaaatcttctaactttaattctgtctttatcttttgtctcctttctgttcctctctgcctgtcattcacaatgaccatttcttttgtttgtataaacttatagggtgcaagtgtaattttgttatatgcctagattgcagagtggtcaagacttttagggtatccatcatccaaataatgtacattgtaaccattaaggaatccctcatcatccaccaccttttcacttcattacccttccaaatctccattgtctatcatttcactctctacctccatgtgtacacattatgtagctcccatttatgaatgagaacagatttgtttttctgtgtctgacttgtttcacttaagaaaatagcctccagttccaaccaggttgctgcaaaatataaaataaaataaaagaatttcattctttttcatggctgaataaaattctactatacatatataatattatataaaattttctttatccaattgttgatggatacttaggttgattccatatctttgccattatgaatattgctgctataaacatatgagtgcaggtatgtttttgatatattgatttcttttcctttggatagatacccagtagtgcccaactacgatttctagttctgtttttgccaagctatgctttcatgactcgttaaatgtgtttttctagtatgtactcctcttacctttgtcttgtatcttccccaatttaccatttgaaagacattaagaaaaaaaaaaaaacaggccaggcatgttggatcacgcctgtaatcccagcagtctaggaggccgagatgggaagactgcttgagcccaggagttcgagaccaacctgggcaacatagctagccccatctctattttcttttaaaaacttatatacatatattactcattttccctctttgtctgtttccctagctgccaacttatggtctgctagggcagtaagaccttctaccattctcataacttcaggcaaatctagataacaaatctcacttaaaatcctcgcttagttttctttccatgacttggttcttcatgtgggatcagcttttaggtacaagataaatgtgtttcctctttctagctcagggttataatcaaagagcaggacatactatccagctaacaggtctagatttagaaaagtcagaatgtactacactgcctgtgtctctcttccaacttccttctcatctgtttccctgccaacttccctgaaactggacccactggcacctgaggccaacagggtcgggaactgcagctctaactttgcccaatagtgaactatggcaatttccattgagcagctgcagccaagtgacgaggtgagctattcattccaatccctctctgcctctcaagaaggcagaagtcttgaacccccaagaatttctagatctttgttttcttggtcaaataggcactgatgttgcaaatataaaaccagaaagagatagaaaagtgtatacaaatatcaactttatattttaatcactctgcactctgaatttttttacttttcttgtctctctcacaagtatgatgacatattcataaatgcagagaaacaacaaaaatcaatttcttatataaaacaaattcattccaggatggaaatttcaggaaggaactggaaaaagagaaataataagtattaggactttcaattcagattaaatttaattctgattgactgcaagtctctgtaatttataaaattgctgatccaaagttttgcatttcttctaaaagtatcaattaagcacttagcataatgtcaagctgagccttctgggcaaacaaagatgaggtacaagatagtccctgtgctcagaaagctcacagtttagtgaaaaagttagacatgtaagccaataaatacagtcatgtgcgaaacaatacaaggatgtacaggtttttttgagaggactgaggagctgacacttgaaggttacctacagaggttgtcaaagaaagaaagcatcacagagcaaagaagatgttaatcaagtctagaaggatcagtaaggccaatgtggcaaagttaggaggcatgggaaggaaaagagattccaagcagagaaactggaagtgtaagaaagcatgggaaggccaagtggcagggcatgtgaatgacactggaaggggagtctagggtctagtcctgaagggacaatgtgtcatgctaagaacctgggactttatccttcacacagttggttttgaaattctgcctcttggagttagagattgataattataataattaaaatgatataatagctaatatttattggatggttattaaattccagaaaccattttaagtattttaaaggcatattcattatttagctcccaatattaatattgaatcctcaaagaaccctatgatggaattatcatcatcctaattgtatttattttagtagtagtagtagtagtagtaatagcagcagcagcagcagtagtagtaggagtacctttgagacagggtctcattctgtcacccaggctggagtgcagtggcctaatcgcagctcactgtaagctcaacctcccaggctcaggtgatccacctcccaggtagcatgactacagatgcatgacactacacctggctaaatttttgtattttttgttgaaatgagcttttgccatgttacccaggctggtctggaactcctgggcctcccaaattgctggaattacagggatgagccactgcacccggccccatcgtaattttatatgtaaagaaacaggctcagatagcttcctctagggtgaaagtggaagaagaaaagaggagacctgccgggaggattggccaggcacggtggctcactcctgtaatcccagcactttcggaggccaaggctggcggatcacctgaggccaggaggtcgagaccagcctgaccaacatggagaaaccctatctctactaaaaatacaaaattagccaggcgtagtggtgcctgcctgtaatcccagcttctcgggaggctgaggcaggagaatcgcttgaacctgggatgcggaggttgcggtgagctgagatcatgccattgcactccagcctgggcaacaagagcaaaactccatctcaaaaaaaaagtaaagaaaagaggggaccatttttacatagcaggtttggtggtgaggatacatttaaaccaagtgttccattgctaataaaaacacagtgtctgagaggacaagaagactctgatcgccagttaatggaagctgtaagaactagaggaagccctggctttcaatactttgtgtcctttgctcatcccattgttagaagggatataaaggagaagggaagggaaaggggaggaaattatacaaggaggagttgacatttcatcaggccctttctggagtccttttgctgttcctctactctgcttctctcctccaatatgagatattagcaaacctagggcaatgcttcacaggtgttttctcatttcatcttcacaacaatatgttgcacagcctgctcatcaccccaattcacaaatgaggaaattgatgcttagagacagtatgtgaatatatgattctttcctgtaatactaaccgtagccttcccctccatccaatctccatcttttttgaccctttctgctctcacccatcctcaactagataacagtgctgtgccaaaaacctgtggattttctctacaaaaattatgtaagtatactatattttacttactgtgtattggcatatattagttgtacatattctgagggtatatgtgatattgtgatacctctatacaatgtgtaatgatcaaatcagagtaattggaatatcacatcaaacatttattttctctttatgtttggaacattataaatcttctagttattttaaaatatataattattgttaactataatttccctactatactatcaaatagtagaacttctgtctcactgtattttgtaccccttaaccaacttctattcatctccttctccccactacctttctcagcctctagtaaccacctttctacttttaacctccatgagatccactttttgtattctacttattgatcttatttattacctctctcatcccctgtaaaactccatgatattttgggggatgagaggtttggttttattttttctcatctacatccctatgtcttgaaaattcttagaagacagtagatactcaataaataatttctgaattaataggtgataaatactcatgaaagcttttctctatctttccaaagtcttaatgcctgctctatttgttttaaattctattgcttcaataagatactttaagaactattctttcataattctgatctgaagaacttacaaggtgctaaattagaagatgtattgcaaaggctttatttcaatttggccaagctttttaataaaagtttgacttgcttaaaagaaatatttatctgggattcgagttcactccttagactaggtcaacactttgagagggggttgtttttgcaaaataaaaattaaagattaagtgtttctcatgacaaattttgaattttatttccatgccctgattcccaatgacttgacactataattgagatattatacagattttctcaataacttcagagtgcatttatatgtgatcaaaagacctttctgatttcaagatacctttaataacctttactaattattaaccaaaactaaaggcagattttgcaaatataaacttgtcatttcagcatgactgttatctttttgatggaaaagatgaagttcagcgaagaacaaatctacaacttggccagaagactgtagactttgccttcaaagaggtgtaacacatacgtaaataatcccatcagaccccatccacagcctccctgctcttgcaatttaattaagcacagtgagagggtttgagagtatcctcacgaaaagacaattttggtagcatttgggaaaaaagcaagagttaatttttatctgaaagactagtttcctttgtaagtcttcaaaatgttttgccttttgcaaagtacagacactgaagctggcatgttaacttactaagttagcagattccccctaaaatgcaatgccctaaagctggacagagggtagaaatagcaaacagagagcattaactatatgcttcttcaaacaatcagtgaatgcaaactgaattgccctgagtcagcagcaggcaagaaccatcagaaaggtgaaggcaagaatatccatgtttatataaagaggatgacattgttctgtccctaaaaacagctaactcttgataatctcattttttcctttgatgttctaaattataatgaaaatatacttccttttttttttgaaacagagtctcactctgttgcccaggctgcagtgcaatggcatgatctcagcttactgcaaccttcacctcccaggttcaagcaattctcatgcttcagcttcccaagtagctgggattacaggcatgcaccaccactcctggctaatttttgcatttttaatagagacagggtttcaccatgttgggcaggctggtctcgaactcctgacctcaggtgatctgccttccttggcctcccaaagtgctgggattacaggccagagccactgcacctggccaaaatatcactttctaaatgatgcttatctattagcttggggacatttatttaattctactacttcagtagtagaatacccattgctattactaatagtagtactattagtattactatagtattactattattacgattagtattgctaatcataatatatatagtattagcatactatatgcactactatagtaatactatactagtatactaatagtagtattataaatactattaatattactattgctactatcatagtattattataattactagtaataattactattactattattatgattagtattactataatcataatagtagtaatagtaataattaccagtaattataacaatatattactattgctactactattactatattggtattactactattagtaatactagcactgtcattattaatagtgctacaattagtaatactagtgttattagtaatattggtactaatattggtgctattagtaatagtagtactattaatactaatattagtaggtagtcctaataatattgttattgttactaatagtatatcttattactattctgttattcaagtaatagtaattttagtaatagaatacctctactttagtaatagaatacctattactatattgttatcattactactattagtgatactagcactgtcattactactagtgctgcaattagtaatactagtgttagtaatattggtactaatattagtgctatcagtaatagtagtactattaatactaatattagtaggtagtcttagtaatattgctattgttagtaatattctattactattctgttattcaagtaatagtaattttagtaatagaataccttaaagctgcatttttcagaaacgatcacttcagcttcacttttttctttcacccaaattttcacccagtttcttatattattaatggtgtcttcattctgagtctttaaaggacaatgtgaagcagtacaggaattctcaacgcatatgacagaaaattttcaaaagtacctatattcaaatacattctagttgcatttgattcagatcattgtaaaacatattcctctgtcctggttggcagagcacattaagcagggtgaaaacccagcaaagaaccttccctgagccactaataactagaacccagtgtggcagccttcacctctgtgtatgtgggaacttccagggcctcacactcctacagcctagaaccttcaaaatggacattatcttgttctgggaacttgtggatgtgagcaaccatcatatttggaatgtgattcaaatcatgttcccaaaatagaaaatgtagacaatgaaatgctgaaattatacatttatatctgtacacacaaacgtgaacatatgtaaactttcatcattttttaaaatttacaaaagttttcatgtactctactgaacatgtctgtggtatgtgatctgggtgttagcaaacccttcccacgtcaagtcaaaagcatccattgttcttgttttgttaaaattaatgtcacatttttattacatagagtactttgcatgatgacttctgtgtatgtgatactggcatattttgctctaaaaatttatctttggagaataagtagataactatcttagctgacacacagactaaaactcaatctctgagaattctgatttcttatcttagtggttagaatgagcgacagtgtcctcttctcacgtctgtaaggaacattcttcatattcttttaatagatgtgagctgatctgtttttgagtatttggtgacatcctgagataagtggattcagaattactaatctgcattataattggagaatgggtattgttcatttttttttgtttctaaatttcaaccaatattgtgcatttttaaaagtttttttttaagagcacatttacacagccttgtttgtatgtaactcttagttgctctaactttaatgacgcatttacagagaatatttcctaaagatgtccttcatgaaaaggagaagtttggacttgacaacagtaatttacttaatttatcttaaatttggtttcagtgagcaaccctaattaacctgattttttgctgataatcactctcaatggaatcaaatcacaaatccggggatggattgagcggcactcagaaggaagcagccctccgcgcactggtccagcgcacaggatatagcttggtccaggtaggaacttgtgaaatctgctgagactcaccactgaatttgcacagtggtgggaatattggaggtcctaagatgcaaaatgggaatggtcagttacatagctctttagagaaatgtccctttctaaaagagaaggaaattgcaacatttctgttttgtagatgacactgaatgtaaccttctttctactctaggttatcaattcaaccttaacttgtcacagaatcagtgtccacagttatagagactatcccaaaaaggcagctagaatttgccacccaaggctatgaagtagaaccacattggtgtttcaggaatcagcagaaaataaactacagcaggaaccaggcttagtttccagagagatcactgcacacctaagacttctagatgtgggatccaaggaatgaactttttgtgtctagctgctgcagtttgttctctcagaacacttgctcatccagtctaggatatgtaatactaacttgtctccatgcttagcattttgtaaaaggaaaaaagaaaacaccacaatcttaaaactgtctatttatagacactgccttttagaaattatctgtatattattattatctttatgatatcttcttttagagaacttatgctgcatttaaaaaaatgtagtgttagatacatatattatcttatttaaagaaaaaaatgttgctgtttttacagtaggacaaagtattttaatttctcagctattttctttcactcacatactttcccaacttaaccaaatcttgaattcaaactactatgaattcaaatgtcatgtgtgattagcaataaaatatgcaagagtgtccttccattagatgctcctttgtcagttgcaaaatatacatttattttttaattaattttataaatcatggcatgctttgttagacatagtaatagttattttcttcatcctaatgaagagcgttagtcttttttaagatttcaaaaagtttctattatttcatagttgactttttgaagtttcacttaagtccatggagatactgacatattagtttttttaatacttaaaattcaaattattttagactcattaaacttttgaaaagagggttttctttctttttgcaatcaacacttccatagatgttatacaatcaagaatgtttttgaatctttttaaaattttgaacttcttgatattagaaacatttgtgcaaagacagtagaaatagaaagcatttcaaccctttcaaacagcaatctgttaatatttatatatgagtgttccttttctggaactgtatttctaatgacccataatataagatagaaatatgtataaatctgtcaagggtgacattagctgtaatgcatatcacaaacttaaatgtataaatacaagaaaattattaagtaaattatgaatgtataattattatacccaacaagcagaatttatgaaaaataatcctaaaacaatataaattatcaattacatttcattcaattctggatactggttcttccttttccccccacatgaaattcaggtaaagtctcataacttttctggtaacgttcaggtaataaaaatccttcatttaaaaaaattgatatttttttcatattgatgttattccaaactagattgtttggaataatagtaaagacataaaatcattcaaagttgcagttatttccctttctcaagtatgtcagggtggaggataatttcttttctgtttttctgcactatgttttcctctttcaacaccagtttggtaactacaatttctgactcctgattgaagcagaagggagaaataaaagcaaagaaaatggtaaaggaaagtttttactgagctaccttcattgggctctcaggcctgacaggtatcgaagcttgtgagattctttttacgatcttcagacacccagcctcctgctgggttctctttgatatttgaagcagccctcatggagaggatggaagataatcattaatttctacacgtggccaaactctgatccccggaaacacacattccctgacccaacaaaatctggaaattgaggtgacacaaagcaaccacttctccttgccaacaacaaagctgcaggccaacccattgttcacagtgagagatttcaggagtgagttagatccaccagcttagtgtgttccccaaacacaaaggcagggaatacacatcaagctgtctaagtggtttctcctgctttaggcaaaatgacaggcgcctcccaattcctcccacttagggggaactaagagtcacaacacaacagcagtcatctccaaatatctttgtgcaaattccaaaaacttctctcaaactctccaatctcttttctaagcttagggtgttgggtgacagttaacaatggaaaaatagtttcctgctaccttctcatcctgcttggtgctctagtcacttggtttcatacctaaatcagaggctagagttaatgataatttattacatatttcaaaacagctagaagagaagatttgaaatgttcccaacacaaataaatgtttgaggtgagggatatcctaattaccctgatttgatcattacatattgtacgcgtgtattaaaatatcatacatggactccttaaatgtgtacaattattatgcaccaattttaaaagtccaattccgtcttaaaatcctgtcacagatgaaatagtattcagtaactaacagtaatcatataaaaaaatctgaaggacaattgtaacacattattgttaaagctatgaagaaacctttcaaataatttttttgaaaagagacccaaataacaatggagataatcttactccatttaaaaattttgctccattgtaaggtcaaatgttatgatttaaggacatttatctgatagagaaaataatgatatgtataattgaaaatgttaacatataaaatactcctaaaatatatggccaatctttattttactttgatcttatctattaaatgatattcagaaaatgcttataataaggacctagaccacaagttaattgtaataataataataacaaaaaatattctatctgctttgtgtatattaactcatctagtccttactatacactgtgtgatagctactatgtttatccccaatttcctgatgaagaaaacaaggcacagacagtttagataacttgtgagattaagattctgttgttattggccaaaatttttggttagactcagaaaactgagagttgttataatgtgacttttaagattgcaattttagagatcagcatcctagagcatgtgacaaacatttttttggaacctaaactttgtaacatgattcactttaagtaatattgactagcagcgtgactgttgaatcagaagtgcttcaataatgtagaagcttaatgttccaaaagctagttatctatacctaaggcctctaatcaaaatgtaaatacctatataactcaatcattttgaatgaattaaagaatccctcctgattttggctacagagtgtggaaaattatccaagactttaaatcagaaggcctgtaatctaatctagtccctatgacatatgagttgtatgatctttaaaaagtcccttaacatctccaggccataatctcctggaagatcaggtacgcaatacccaactatttgtccatgatttttgtgattctagtatcttctgaatctagtcactggttagcacactggactatttggtcgaaagggtagcctaaatatgtagaaacacaacttcaaactctttgtaacttagggaaaatcctttagtcttactaatacttcagccttcctactctggattatacaaaagatcaatatgattatccaaaaagaaagaaagaaaggcaacattaagaacatcaagtttactttttaaaaaagtttttgtacaaaaattaagataaagaaacatcctaatacaatccaagcagcatacccataaaatgatagtttagaattttatagattagggtttgaaggatttacttttgctgtatctttgcagtctctgatattgaatgttgaatatttacagaacaagaattcaaaaaacagagggtgatgaatttgtgctctcttatcactagcaggacaaatacagctatcaaaaatagaattttgtgagttacttaataacgtgacccaggcttttatgtagcaactgatgcaataatgcctgcttctaagggtttggaaatatgctttgtgatctttttaaaataatcaaacatctttggatattgatgactgaagtttttatcaaagtactgtagagaaaaaagagatataatgtagtaccccacatatagtatgtatttaatacatactcttgttgaaaattgtataatattactaagttaaactttaagcttcataattgtattatgccttataaaaatattttgtaaggaagtgtgctgcagtattaacatgtgaacagtctttgtctctgtgtaatggaattatgtgtggggattcttttctctacttttttattcttaactgtacttcctgatttcgaaagctagcatatacttctttgatagctaaagacaaaaaaaattacattatgatgagtctttcctcaagaatgcattaatgatgtgggtcataaactatgatatgtgatgctcctctcacctgaaattttctccttagaactagaaaaatatattctcatatgtgagcatattcagaaaaatatacacttccacccaacgtggtgtctgttgacatcaagtttaattctattgcaactaaacctaaatatatatttaataaaaatcaccctcaggtagtcttaattttttaaaaatagactcattatatctaatatatataaaatttaaaatgtggtgtgtattagctttgtttatctagttatcctattggtagtctgttttctaaaaatcattacagcacaatcaatttttctgaggtgtgatagtccccttgctttcgtaaaccaatgatcatcctaaataaacatcaatattttttgttttgttttgttttgttcttctgttttgcttccttagtccctattattgagaaaaatgtattagtgctgggtctcgggattatagagtataaaaatgcagtgcctgacctaaagttcatataaagtgggtgaaatgaaaaagaatgtatctaaaatttacaacaacataaggcagcctcaaatgagcaatacagctgtgggtaccaaaaagatcagcattttctatatagagccttaaaattgacaatgctctttgacctgaggaatgaggacaaatatttagacataatgatgttattatagtgaaatgtataattttttagaaatttaaacttaaacatatcctaaatatccaatgtaatattaatggtcaggcaaattatgatcattcttatattttcaaattatacagtaatataaaattatgatttcaaagaattttcaaagacatggagaagtattcaattttaacatttagttaaaaatctgaaggctacatataaatttgtgaagaatattttaaaaaattattcacatacagtaaagaatattgtaaaataacaaagtataatctctaagtttttcttgcctgatgctcttcttcattttctaaattttctataataaatgcagatcgtttttatcacaaggaaaaaatctcataaggtagaaaagatttctaagggtcagtcaggagttaaaaaaaagaaaagaaaaaaatcatggaaaaaaactttattattaaatttgggtgataggtccctaggttcactagactattctttctaattctacatatacatgaaatttgtttaatattgatatagtccaatccttttgttctgcagttagaaaataagccccagaattacacaattattaatcaggggtcaacaaactatgccccatgaaccaaatgtggctacagcttgtttatgtagtaattgcaagatatgattttttttttacattttaagcattttttttaaaaaaaagcctaaaatatcaactatttggccttttacaaaaaaaaaaaaaaattgctgactccaactcgattccttagcctactgctctatattagaagcgaatctagtttttaaagaaaattctttaatttagtatttaatctcccttaaaagaagtaggtgcatcccttcttgtgctgcccattcacaccacctcaagcactgggccctcactcgttcatgtattaattcatttcttgattccaccacaaagagagacatgtttctacctttatgaagattagtctcatagaaaagatagaaaattaacagataaagaaacaagcaaagtaattacagatcatggtaagtgtcaagaatgtgataaaccaggctgtgtaaaagtcaaagccaggtgaccttctttaggaaaaattacattgaagctaagacttaccagatgagagcaaacaaactctgcaaggagccaagtatccctgggagatggagcacacaggcagtactccttaaacaaaatatttatacagtgcagaacacttttgggtagtgaagacattaatgtgtcttactttcaaagatctagtgagattacagcagataagtcggtggatttcatctttcccttcctctgttaatcagccatgaacctggtgtcctgggaacaatctcctcaaactaggaacaaaatacagtattcaattcccacccctcacactttcctttatatgcattctctcttgtatggaatttcaatgaaatcacatgttttgattctaatttgttcagttcttcagcaaatacaagccatcaggtacaaagaatggagctagatcaagtctaattagaataccgttcctcttctaagagtgccccaaattaagctaactctccatctttccggtttgagataatgtacttaggtaattttaattgaatgttcctcaaatggcattttttcctataaataatacaataaaaataacctgagggtctcaggtttactaattggcttcatctagactggctccgagaggagcagatggaaaaacactgtggcattgacattctttttcaaccactgcacaaaaaatcctagggacagaaataggaatttgacttcatggcaaaatatagctcctgggtcctactttttcctctattgcctgttttcaccacctgctcacatccctgctatccaaggacttaggtcattcaccaagtgcacggataccaattctttaaataggtagcaataagcattagaacaggatgggaccagtagattatcacaggctcatgcttccctaagcttatcaccccagccacccacaggccaactttattttaaagctcagaagcagtcttacttctactagccagagatgccatagcgtggtgtgaaagactccaaatgaaggagcagttctgtgaatccctttgtggagggggtgtgtgcccaacatttgtgggaacttcattatcccaaggaacacagatatgttccagaagctattatttccaaatagcaatgaatttctcctcgaatagcctcatagggtgttgactatatatgaagtgtttgatataagattcaaaatcttcataaacgacacaaggtagtgctttcccttaagctagaagcacagactttttctattttatcagcttaaaaattggtgtgacctcatgacccaaatatgcagaagcatgagcaggtcttgtggactttggactgcgcacgctgacaaattcacactttttcttaaagtgctattcatttgtgagtactgattggcaactaaaattaagtgactcttcatcttcagaacctgaaagcttagcattatgagatgcaaaagacagctttcaaacaagctaggcaaagaagaaattattgagatcacagttccttgcaatcatgaatttgtataggatttcagcagctgtgatcctttgtaacattgttaaagaagtgctgagcagggaaccaagatgatgttaaatggcacattgtccacatcattaaataacatccaatcccatatagagagaattattccactttcagttctctctcaggcttccaggtgaggaaagttagttgttggaggtatgatttaaacacctcttcaggccctgctaacctttttaagtcccgagagcagaactagagtgcagagcttttagtagacagtgtctatgtagaagttgaccatgatttcccttgtttgcaattgtttttataatcaccgaccatttatgggaaatgatactgatattccacttataagaacaatatattttctttttgatataagcttatttaagtaacaaagtgaatcaatgcataaagtcattaggtaattaacagaatagatgtcaattggacatggaattgaagatacactgatttttaaaattacaatatcctcttaaggctccaattgactaactcacaaaacatcctatgaacattttcctccattaaacatgatatcaaagtcagccttccttttacaagcaccctcacacccctacaggactactgcaatcacctagtcaaccctcctactccttcatagatgaaataattaaagtcagaatagatttagtgccttgccaagtccctcagcttgtcagaaattaaaattcacaagagtgttgtaaggagaagaatgattatacaaaattccttacatgcctcaaacccggtgcattggatgggattatgtttggcatatgttgaactaggaaaagatagaagagcaattacccaaaccaaatgttctttattatgtctttatctgcacatgggaagttttgcataacttaatcagatctagcggccataataatagttaattcaaaaaggcaaattgtaagtaaacaaaaaagttagatgggcctattgaataaattacacattctttaaggataaaagctccaaacggattaaggaatgactaaagctgtcacctaattacttcttgaaaggaatttagaggttcctatagatggaagcagtgtgtctgggcacatgcccagagccagaagcgagcatgagcacagcaattcctggcctcaaaaaaggaaataatgctttgcagtccatcattttgcaaactttgctggaaaaggtaagcagagggtcttagaagtgagttggcattttactcgttctcttttacagaaacacacacacacacacaaatgctttttgttttctaattttgaaaaacactcgaataagttttctgttttgtaattttgaaaaacactgacaaaatgttttctgttttctaattttgtctattttcaaattcaatattcttatgaatgaccaaaatcttacattataaaaagctttgcaaaacacatcatttaaaaaagcctatttgcttttgaatgcttcaaacgttgatactgaataatccttaaacataaagggaaattattaaaagatattattaaatgcatcaacttgaaaacataaattacttctgcttccataagatagttaacaacttcaagtgaaattgggattaatttccttggaaatagactttcaatggtcaatcttccaaagtgaaacaggaaggtgactcataatttactcatagttgtttccttagaagaaccatatcatacattttttaaagataaaatgtcagggctttgtaaaaagtgaaaccaataaatgaaagtcaagagccaagagccatttgcatctttcttcatgatttttgccatagatacatgccaccaatattatttatttagaattcattaaatctcctggctaacacagtgaaactccgtctctactaaaaagtacaaaaaattagccaggcatggttgcaggcgcctgtagtctcagctactggggaggcagaggcaggagaatagcgtgaagccaggaggcggagcttgcagtgagctgagattgcgccactgcactccagcctgggcgacagagcgagactccgtctcaaaaaaaaaaaaaaaaaaaaaaaaagaattcattaaatcaactcactatttttagtcgtttatgttaaaagcaaactttttctcacatatgtaagaaacccacaggttttgtgtgctaattatactttttctaatgtacattaaaataaatgcataattattaaaataagaaatactgacctataaaccatctaatatcatcctcacatattttgagaaacactaggctatatgatgataaagctaaattaatatttgataatgttcatatcgtagcttttcctgtctttgtttctaatctacatttccacaaaatttcaacaactcatttagtctttatacacaatttttattattcttcctactttaggaacattatatctggcattcaagttgcatacatcttgtaaaaattgtagaaaaatagccaatagaaataatgccaacttctacagtatattaagccaaataaattctgctttctaaattccagtctaaaaacagattttaccaacatgaagtcactgattttaacaagatgtgaaggtgaacaaactatacaaaatagataattattttatatacaatttagaaatgaatattccctgcagcactgtgtgttgttctcaggcaacaaaaactttagaagagaccaaaggtaaaatcacattgaagaggatcctgttggtaccttttatggccccatagactttccttaaactatctatcacataatgaaggatctagtatataggtggaatacaatagaaataatgtctaataaatacttttattcaatttcacaaatcaacaactaggaaaatacagtatagactcatgaaacacaagtgacacagtcaagagtgatgtcctccatgccttgttgtcaactttaagaaccatgcacaggagtctgatcacagaaccactgttctgggtatggtcctggtgactggcaagctggtaaaatcttgaaatgctactaagcaagggtagatttggctaggacccaagggaacaggagcaggaataaatactaacctggtcaattggaaaatgtacaggaaaacatctgaaacaagtttagttatgaaaaatacacagtttcttccctcttagagagaggggagaaaaagaaacaaagaatcaaagagaaccacaaagacgattacaaacttgaaatatgaactttccaaagagccattaaagaaactgaaattactactcaggaaaaaacaggctgaatgagggttcattaattaatgtccttccggttatgaatgggctcatttatgggaaacaataaccatctggtgtccatgattggtgaagttgagcaggaaaataaatgtaacacacaacatgaggatttatgaggctgataagcctgagaagaataatcatcacaatttttgagtactatatatgccataaagttcaccaaattgagaatgataggacctttattctagtcttttctacaataaattaaaccagtgtgatattagacaagttatgtatctctggacttagtttcctcatctataaagttagtatattgtgttaatgtaaaaatgaaggaaagataatggaactacaagcgtcttcatattttttctctgctgtttactgcttgtatgaatttggacatattttatgccttggattccttgcctataaaatggaagtaatagtagtagcatcctcatagatttgttgagaagattaaataaatgtaaagcactcagaaaaatacctaattcaattggagatagttgttgttttttgctgtcattactgtcatgtgcaagtaacagaagctaactctgactggctaagtataaaagagtttattaaaagggtactggttcacagaaactttagatgtggagaatccaattcaagggtaagcttttaagaacaaaacccaaaaccacatcacataattgtcatgataaggaaatgttatctgccatcacagaatgctcaacattaaagtttgcaccatttgtggcttttgcatcagaactcaattttagtaagggaaaccatgattgtccatggtttagtaaaggaaaccatgattttccaataaccatgatggccccagctgcaaggaaggctagaaaaatcaagtatctgtcatttttaagttctataggagaaggcaatttttgcccagattccccaggtatataacaagttcaaacgctcagtggccaaaattaactcagttcattcaatcttcatgctaagacttactgcttagaatgatttattgaggaaggttttgtggcttttatccaaggtcaagaagatggtagacaaaatctcttgaagactcaatggcttcatcatgaaggacttgaaatatctgtgataggaggaaatttccaagtactcttgacatataaactgagtaactgggtaggctcagcatccaatatgtaaattaaagccactggatttatagttagaattgtgtcctcaattgttaatttacataggtgaagactttccacctatttgtatatgactctagagagacaacggtttgtaagtctttggaaaaataaaagtttccagagtttaataaacattctctcatgaaaaacaaaaatctatattcttttagtctgaactattaatacatttgtgctgctggggagaatgcttcccttgaggaagacatcagctaggagagaaaagagcagttataactgggctttatctattttaaactcctcaactatgaaaatagtacctgacatattagcaacccagagcccaactgtcctttctgagtaatggacagctattctaaccccatgactaatgcccattgcctgtcttaaattgatctgaacttccattactggtacattccaagaccagcgggtggctcacagagcaaatctcccctactgagtgaactacagggcatccctgaactggccagactctttacagtctcaaaaaatattctttggattttttttcaagtgtttataaaaaacaaagaaagacaattaatatgctttcttgcccagttatttcagattgtaagcaggtaaatcttcctatatgcatcatatgcataaatagttggatggcatttttagcaattatagcaatcaagaaagttgaagatatatttaaacaggtcaatctgctggttgcctatttattttcaaactatcaaagctattcaaagcagaatttctggaattaccattttactattatgagattaaagtttggtttcctcaacttgaagggtaaaaataccacccaatgtatagtaaacttataatctgttgaatgttacagacatcaaagatacataaaaatgggctcagaagcatcattatcaccgaaatggaaagagtttgcccagggagaaggatggtaatttggcaattcaggttgagtatcttttatccaaaatgcttaggaccagaagtattttggatttcagacgtttttaattttagaatttttgcatatacattataaagatatcttggagttgggacccaaagtataaacacagaatacacttataattcatatataccttatacacatagcttgaaggtaattttatacaatgcttttaacgatttcgtatacccatcacatgaggtcaggtgtggaattttctacttgtggcatcatgttggcactgaaaagttttgaattttggattttggggttagaaatatgagatatgttcttccaaatattttatgtaataaaatgatcggaaattgaaaggattttaaattgcagtagagaatttgctatttaatggcatgatacattttttcataaataaggagttttttttttttttttgagacagagtcttgctctatcacccaggtgggagtgcagtgacaacatctcagttcactgcaacctctacttcctgggttcaagtgattctcatgcctcagactcccaagtagctggaattacaggcatgtgccaccatgtccagctaatttttgtatttttagtagagatggggtttcaccatgttggccaggctggtctcgaactcctagactcaagccatccacccacctcagcctcccaaagtgctgggattacaggcataagccaccatgccagaccaataaggaatattttaatgtatgtatctgttataagtggcttccacgtatcaaaggagcaaaatgcaaaaattaagctgatgaactttgatgcctgactgcctgggttcaaattctggcttctccacttcccaaccatgtgaccttggcaaattaatttatctgggctttgataccttatgtaaatgggataataactgtccttaacccatgacattaagtaatgacagagcagtatctgacatatagtaaatgctatataggtatttgtttgaaaagaagtaaataaataaaactcattaatgtctctgaaaatgtcaattgcacatttttaaggtaattcttggttgagatataaaccactaaattaccaaggttttggggtttttttctataagcataagtatttcaatttctcagatttctcagtgatatcaaagttttactaatggacaagaatattagggtaagaaaaaagtacaggaactctctaggcaaaaatttaaatattcaattagattttaaaaatactcagttgtttgaaacaaaaaaagtaatccaggcaaagtagaagactcatatgatatctttttaagttattatttttaaaaaacgtggtatgaattattagaaataatgtgtgatcatgacccacagacaccacagatatgaattaaatactgtgatccaaagagcctttgtgctaacacttacaaaatataattgagaaagactcaagttggtgtatatgaagatttaacagggacactacaagctgctttacttcaattttaaacaacagtcatacaaagcagtgtatttaaacaactaacatatgtcttttaaaaatatacatagaaaaatatttgaaaaggcctatgaaatttttgacagtaactacttctaaggatggtacagtgatacattggagaatttttctttttactttacatgtcactgtgttttttccacaacttgtatcattttatagcatgaaacttcttaaaatattagttgtagaagttagtagtgaactcttgggctcaagggatccttctgcctcagtcagagtaagttctattgttctatagcactgcaggatgactacgtttaacaataatatattattaatatgtagttgtaaatagctagaagaaggctattccttgtgttcccaacacaaagaaatgataaatgtttgagatgatggatatactaattaccctgatctatttactctttaatgtggtaagaagatagatctcttaagtgttcttactcaatgaaattaaaaaagaaaaaaaaaatatttgcctagcagtttacaagttttcacaatgtttatctcaagcaagctgctgaagcacagagtgtggtactataatccacataaaaagctttaatttttctcctttaaccagtccactattattacttgtcaatgataaatacattcattaatgtgaaattattttgaaaaatactcagggaaattgaattaaagtcttgatcattttctcctctccttcacacctgccctggatttgcaggaaaatggacaaagaaaatatggtggccctccacctggttgggatgctgcaccccctgaaaggggctgtgaaatttttattggaaaacttccccgagacctttttgaggatgagcttataccattatgtgaaaaagtgagtccaaatttaacatttaatttttcttatcgttcaagggaaaaatactgttctcttcactttgctttcattctcagccttcttggtcagtggctgtttccattccaattaccattcctcaattatagacaaagtttctagtgattatctctttataatcactgccttgacctagctcttgtcctgttactgacagcttgaattgctacaatttctaatgactgtcaaatctatatcttgttccccaatctttccactgtcatgccatctgactacaaactatccatcaacaataaccctacaataacctgtctaaaattagatattgctccaacttgaaattttaatacagttttataaagtacaacattttcattttaaaagaatgtgtatgtttgacaaggctcaacatgtatctataatccagaatgaaatctccaggtctgaattttttgtctacctaaatgcatcaagaagattaagaacatggcttttcagttaaaggaagaatacaaaattaaattttatttttaataatttaccaagtagtttttagcaacaagcacattcctgaaaaatttagtaaatataagaatttagctctctgcctgaatgccaaaatcaattttgtgtgtgtgtgttaacagcctcagattcagaactgcacctaattttacacatacaatccctcttggcatgaggacaaatgatgaggacttaattctgtaagaatcttatatactgaatctgaaaatgagcatcaagaaacacctgaaacatagagatgtaacatgttcaaactatttcctaatgagcccagaagaaataggtgttcattactgaatacgtgagggtgggctagggtaaggctgagctgagaagtgaccaaccacaaatggcatgtgcttaatggcacttctctccagacatgcgccatcatatttatttgggcaacccttcttaaagcagccagtgatagtaaattgctcagtctagattgctgcaggacagttgtggagtagaagggagtattacatgggcaagtagagtcactatagccaggggaatagagtgttctcctgctctggaggccctagccctgagttagtagtatttaggctagcatttacattcagccagcagacagagaaaatgacaagcctcccagatcatcccctgaaacatatctgtgttcattttgatgtcatggctgaggaaaaagactgctaagaagttagtttggcgaatgagcattcttggattttattttaaacatcaatgatagaataggaccaactggaaattaaactatggagatttcagaggacaatttagaggaaatggacatatacatcaaaaaattgcactttttttgagacagtgtcttgctctgttttccaggctggagtgcagtggcacaattatggctcattacagcctctattgcctgggctcaagcaattctcccacctccgccttccaagtagctgggactacaagcacgtgccaccacgctcagctaatttttgtattttttgtagactcagggtgtcaccatcttgcccaaactggtttcaaattcccagggttaagcaatccacctgccttggcctcccaaaatgctggaattacaggtgtaagaaactgtacttatgataaaatgtgttttcatgtctctaagatgttccagataattatgtgataaaaggtcatttgccctaccaaaaacacattctgttaactcaggctgaaatgtcacttgtacacattttatttaacttaacttctctcatgaatcagataactgatctgaacccacatgaaaatatgcaaaccattccacatgggatgcaaatgccaaatagtactggcaggaggcaaacttcagaaagcattgtgtgtgtgtgtgtttgtgtgtgtatcagcacatgcacacatacattcagaagtacaaacgtataaatataaatgttctatttctattacttgcagatcggtaaaatttatgaaatgagaatgatgatggattttaatggcaacaatagaggatatgcatttgtaacattttcaaataaagtggaagccaagaatgcaatcaagcaacttaataattatgaaattaggtaagctccgcctcttctagattaaaagtgaaacaaacaatgcacctgtgtctgtttttctatcaagtttatctaatatggtgtatgccaaaaaaaaagccaagccagaaatgaaatagattagtatttttccttggcaaaaataaatttgattaaatatttattaatatcaatactatatgaataccaaactaagttctatgagtgttcaaagaaacaaaacttaataatttgcctgtttcataaatatatattgggcactgttcaaaatattgggaatatatcaaggtataatttcctacttgcagagcaaatatataagatcactcaaaatagtatgaaaaaatcaaaccagggatgggataaagttgtgtgagaaggtggctactctaatttggtgtatttaagaaaatactctctggcagagtgaaacttaatctgagacttgaatgaaaagagccagtcatgacagtcagaccttgggaaggttatttcaggaaaggagagaacatcaagtataaagttcccgaagtgaaaacaagcgtgatatgtttgaggggcagaaagcaagcccatgtggccggagagggtaaagagaggaggactgaaagcaatacagttggagagctgtgcactgtgtgaggaagtgtagatgaagtacagaagtctctgtggtgatgaatttgaattttcttctaatagaagggaggaaagagttttgattacatttatagttttttaaacattcctctgctgtttggttgagaatgtactatagcaggtgaggtttgaaagaggacaccagttagaaggctgctgcagttgtctagatttggacaagagatggtggtagttggatgagggggtccatggtggacaagataagaaagggtgagatctgggatatacacaaatctggctagagattggatttagactgagagagaattcagaattcagggaggaaacctaggattttggtccgagtatctgtgaacttgttagtgccattgcaatggaaaagactggaagaacagcagaggaaggaccttttagggcactttacatttgagatgcacattgaacatcccaggggaggtgttgagaaggtgactgtctgcattgtggtggagtcaggagacaggtcacagttggaatataaatttgggggtcagctaaatatagatagcaattaagaccatgggactaggggagagtcccatgtaattgactttgtgcaaccaattgcccgttttgataaactggaaataaaactgacatcatgaaattgtctgatttctaaaaccttcacttggagagagaaccgtcataaattctttcttaaaggaagccctgagccacgtgagaggaagattagtgcagcagttaaagagcaatagttttggagtcagccatgcttcatttgagtgccaccaagtaacttcaccattctaagcctcaatttcctcatctataaaatggggataccatctatttcataggtttattgctataacaactttacagtgcttaacccagtgtttggaacacagtgataagccgacaaatgataaaaaaaaaaaaattgtattttagcacagttcatcaattctagatgcagtttttttttttacatcttatccccttctgaaagagaactgcaccttaaaatgggtagatcttaaatttgatgaatgcagaaagagcagatttaccacaattatttttctctcactttctataggaccaaaagaagtcatacattatccttacagtctgtaaatcaattaactactggcttattttattaaaaattcctgggccggacacagtgactcacgcctgtaatcctagcactttgggaggccaaggcgggcggatcacctgaggttgggagtttgagaccagcctgaccaacctggagaaacctcatctctactaaaaatacaaaattagcagggcgtggtggcgcatgcctgtagtcccagctactcgggaggcaggagaattgcttgaacctggcagggtggagattgcagtgagccgagatcacaccactgctctccagcctgggcaagaagagcgaaactctgtctcaaaaaacaaacaaacaaacaacgccttagccacaggaagatcagttggattcggcaactacttgtagtctaaccattcaattccaaggctggattctagaaaagagtcttggagaaaacataaaggaggcttccctcccacaggtgcctccatggacccaaaagagctttgtgtagtatgtggaggattgtttgcagtaggaatcaggagatctggggtccagcccaggttcctttggacgaatgctttaacctctttgagcatcacttttatcattggcacctaaacacagagatgttctgagcattcaccatatgaaagaatatatactaaagtgctttgaaagggctacaaaacgctacattacagttttatgatttccagaattggaagtttcccccattccttggtctttcatggaaccttgacatcgtaacagtaagggttattcccaaaacggtatttgagttgagatagatatggaattggctatgtagggaatcaagtggatttactgtatctacttaacacattgttacccaatagggcctgtaaatacttttggacagcagataggatatgaataatgaataaaatcaatccactacggccactgataattaacagtatcctctgtgaattttagtgtctttgtctgtgaaatagagataaaaatatacctacctcatgaggtgttttgaaaaataaatgagataatatacatacatctcagtgctgacacatactaggtgctctatgaatgtttgcagaaggttatggtggtgatgatggaggaggagaagggaaaagaaaaggaaaaatatgacagaaattaggaagagaaagttactggccaaaattccagtcaaagcacaggttggatgccagaagcaaggtaaaggcagagtgttaccttaaaaattagggggtagaataaacggaataagacaagccagaataaacaccccatagacaacctcacctacttgcactctgccttgggtcagtcctgccagggaaagaacttttgttgcagagaagaaaccttcaatttgcttttatccaccatttctcacccacctatcattatatcagcttctacttcatcagatattcactgatcgtcatctaaatgtctggcttctagagacattaaagttgtggcctcagcttttgaaaggagatggctctttgtctccaaataattccgggaaaggtgcctgacaaaccacaaccatttaaaaataaaatatggaattctaggctcctcctgctggtaactgatatatgaccagccccctaactatcaaaaataatataatttctaaatacattaatactgtggaaaacaaggttttggtgttttcttttttaagtaaagtttaaaaaaaaacttggaagattattttttcaacccttccctcaccttgagaaccacactcctggcacaattcttactagactgagatggacctgtcacagatctgacacacggtccttggttcttcagcaaagcgtaggagacagctaggacagtggcctaaccctggtcagaaaacctgccacacaaccaagagccattcgctccatggatatcagaccaatcgcccactcacctggctgagagcttttattcatggcaaatgggtcgtttctgctaagcatacttcacccatttttaataacttagtgttattacaaaagtcatatagcctaatatgtaacacatggaaaacatcaaaaagcataaagtaataaaagttaaaaccatttctccgcaactccattgccccaaaataaccactatgttgatattttattgtctttccagatttttctaaacatttttaatcaaaatggccttcacatatacttattttatagccttttaaagcctagcagccaattgtgaactgtttttttaaaccaatatatattcttctacgttactctttttaacgactgcagagcgtgcaatggtatgaatgcctgtcttttgtttaccagtgattctcaatcaggagcagtactgtccacccaagggggcatttagaaaggtggggaggcacttttgaatgatcagattggtcactattggcatgtagtacgcaagaactagagatggtaaacgtactacagtatgcaatgagaaaatgtccaatccaaagccagtaacatctcagttgagagacattaaaccagttctttaccgtgaggtattttgctcatttccaatttgggggagattttaaagagcagtaataaatatccttaaacataaattctagggctcacttttattttccagggacatattcccagaagatactttatttggtcaaaggttgtgcacacttaagacttttgatacattttgccaaatgttctccagaatagtttaatccatgtgtactctgccagtgaaggtgtacatacctcaacttgctaagaagcaactctgtacagtggaagaaacaggaactgagatcccacagctaggtttcattccttaaaatctataacattttaagcaagtctttttatttcttggagtttcaagttcatctgtaaatggaaataacttctactctgccgatctcacagtctttttgtgtggggcagataaggtcatatgtgaaaatgtgttcaagtataaaagactgtttttaacagagatcacatcagaacagcttttctctttggctacaaaagaaaaggaatgtcaggtgttgcaaactcaagcgactgcagggccaggcaggtggcttcagtaacctgggttaggtgtagggacctcagtgttggggatgcttcagggagcggtgaggattgacaactcttgaatccacatctagcctaaagaaagcagatgctatgcagccatgaaaaaggatgagttcatgtcctttgtagggacatggatgaaactggaaaccatcattctcagcaaactatcgcaaggacaaaaaaccaaacactgcatgttctcactcataggtgggaattgaacaatgagaacacttggacacaggaaggggaacattacacaccagggcctgttgtggggtggggggaggggggagggatagcattaggagatatacctaatgcaaatgacgagttaatgggtgcagcacaccaacatggcacatgtatacatatgtaacaaacctgcatgttgtgcacatgtaccctagaacttaaagtaaaataaaataaaaaaagaaagcagatgctactcagcttcagataacttttttgtttgaaaatgtagagcacatgttgacacatctaataatcttgcaaaagaagccacaactctccaaaaatggtttcaacactatgaacccaaacatcccacatcttcaggcaacatttggcccacagccatacattttcaacctgaatcctacaaggattctcatcaatcaaaatccctgctctttttgcagaaatgggcgcctcttaggggtttgtgccagtgtggacaactgccgattatttgttgggggcatcccaaaaaccaaaaagagagaagaaatcttatcggagatgaaaaaggttactgaaggtgttgtcgatgtcatcgtctacccaagcgctgcagataaaaccaaaaaccgaggctttgccttcgtggagtatgagagtcatcgagcagctgccatggcgaggaggaaactgctaccaggttagcaatcccctcctcaaagatgagacttctctgcctatcctgcctgcctccgcaaagaaaataagcatttttaattttttttggctatctttcattacgctgtattttaaattttttatccttcttcactgtctcacagttgattccattccctgaagttttcagactcagttttgaaaaatattcattctgtgcattgaggctaatttacatgcactaaagaaacccagaactttaagtacagcaatcctttgatgaataaagagtcttcttaaaattaagtatatcaatcacagttacttttgtgtatagattttgttaccaggcttattagaattagggtatattctccattacagtgtagaaataaaaacctcaagtgataccttattccaatgacaactaggtgacatactttcattatgactccctgttgtctaataatttctgtctaatttgagagaagagagataaaagctctccttagaaagccacctttatttgttagttattacaaccacaggatagatcccatttggtgaataactatcaaattctctatccaccccaacactgcctcggtcagatcaacttaggaaaattttttacagttcttggctcattctaaaacattttctacaacttacacaggtttttaaatgaatactaaaataagaggatttcaaaaatgacagactggacacactggaatgaacttttctcatgaaaattatgggagagccaccagaattcaaaacaaaacaaccacaacaaagatgtctacatttatgttgcctttgtaaacactcttcaaaacaaaccaatactagatcagccccctagatatcttcaaaactaggagacagtttctggcttggggaagatctttcaataatatcttcatgaaggcaacttaaagggaaaaaaaatgagacagtcccaaaggttgctgaatcttttagagaagtagatatttttaatgttgaatctttcattcacaacttcttccctcttcactggccagcatttggtcaagtcatctaaatcacaggtccatagatagactcactaagactcctcatgggggcataccttttggtgacctggacgtaatattttgctatctgcttcaattcttctttcttcttcaacatcgacaactgtaccattgaaaagtcttttgtatccatcctgttcttaggccaccactgaagtctaaacacacatattctaaaccttatgtaatttttttcaagatctaattattctcccagctctgctttctcttccatgtgccccaccctccgacttctccatcctgaacattaccaccagattaatgtttccaaaatatcctattatcctttcaacaccttcttgcaaatagtaggaattcaatctctatctataggctgaatgatgtctgtcactaactggccagttcaaaaatattcataactccttgctgtgtgtaagaactaaatatgtgatttcattattcaataccttttattcatttcctggtcttttatgaccatatttgctaccttctatttcccaataaaaactcttccttctatcaggacagacacctcaatgacctagacacttccatgcttatgtctgtctcgtttattcctaatgctatatcctctactaggaatacctgcccctccgtcacttgtgacattcaccccagcccagccagcaccttcttgagaaacctacctccagtattccagcccgaattagtcactttcctccctcaatgcaggaagccattcattatctgtaaaaacagatatgtgaaataatttggggtgtgggggagtaggacagtgaaagaaggtccaaaacaagtgaaagacagtttccagggtcaagacaatccagtaatcccagagaagctccacctcctgaaagcttccctatctcttcagggagaatggcacattggcccaccgaaggaactcctttttctaagtcaagcagactagctagccaggtcctatcagataagcttcagaaattttcaagcacttcattaagcttccacccacagtcctgacttttccttgtggattctctgtccaggatatgccatagggaggctgcctcccctccccgtccacaactcatattcactatgatccaatgcaacccccttacctatggccctttgagccagtctcaggtggatttcactcagctggattttagtcagcaactaactcatcagcaaactaacaagatgaggcacagtggtgagtaagccagccagagcaaacttcttcacctctaatgagatctcagaacaaaacctatcacccatttggatcactctacctgttaccccattgcagactcacccacaacaaaggaattctttaattgccaaccctttgagaacctagtttaaaagaacaaggagactgttgagaaaaatgataagtgaagacaacaagaaacttttctttcttaattgacccagacttggagacagagagacaatagttgtctaaagggatgtaactgctcctcagtcacttacctcctctctaaacagtggcagttcccccttgagaaatctcacctttattgagctctttacagaacaaagttgctgcatcccactcagaaactagaaaggtgttctaagaatgctctgaagaaaagtcctggcagtgattctcaatgtcaggctgcatcagcatcacctggagggcttgttatagcacagattgctagatcccaccgccagagttcttgattcagtaggtctggtgtcaggccaggtaattcgcatttacaacaagctcccaggatgctactactcccaggactgcacatttgaaaacccatgatttgaatcaatgattttccatcctaggctgcacattggaattacctgatgttttcaaacaatgtgagtacccatgtcagcctctaatcaattgaatctgcatcactggaatataaagttagggaactggtgttttaagttttccagatgattctattcttcagtcaaattttgtgattcacagctaaatctatcagctaatgatactgggtaatcatcttcatggcatgtaaataggaatacctgggaactgctcagtcagtaaatatttattaagcatctagaacacatattgctcccttctgaatgacatgagagattctaaaaataaaaaaaaaaacatagtacctaaccattgcactcaaagagtgcatattctgagaaaactatacatacacaaaaacaggaaaaattattaagaggagagtgtattgttaaatcataatttaaaagttattaagtcttctctgcatcatcttttcctgacctcacaacaatagtaggttgtagataaggaaactgatgttagaaaggatatgttttctagccaggatgattgttctgacatttgaacttgtcttacttctagctcatgctctcaaatcactctgtaacactcactttcaccagaggccttcacaaggatgttggtggaaatcactgcttcccatatttactctcatttctgttcccttagccttatgcagctcttcgtggtcctaggctgagaagactaaagctagagcagtagtactcaactgtggctgcattttagaattaactaagctacttttaaaactcctgagatccagcccacttatatcagaattgggaagggtaggtagtccagaggtcagtaacgagctggtaaatctttaacagccagctcccagaggaaaaaacaaaagccctgatttgtagcatttgccagtttccatggtattaacactcccactgtggctgatttcaggctaccaatgcgactttaactggctcacaaaattctgatactttctttatctatttatctgtcaatgaacatttaggttgattccataccatggtaattgtgaataatgcttcaataaacatgggaatgcaggaatctctttaacataatgatttcatttggggtgaatacatacccagtatggtgattgcgggatcatatggtagctctatttttatttttttgaggaacctgcatactgttttccataatggctctaccaacttacattcccatcaacagtatgcaagtattctcatttctccaaatccttgccaacatcatctttgatctttttgataatggccattctaccaggtgtgaggtgataactcattttagttttgatttatatttgcctgatgattagagatgttaagcattttttcatatacctgttggcgatttatatttcttcttttgagatatgtctattcaaatccattgccctgtttttgttttttatttttgagacggagtttcactcttattgcccaggctagagtgcaatggcatgatcttggctcactgcaacctctgcctcccgggttcaagtgattctcctgcctcagcctcctgagtagctgggattacaggcatgtgccaccacgcctggctaactttgtatttttagtagagatggggtttctccatgttggtcaggctggtctcgaactactgacctcagatgatctgccagccttggcctcccaaagtgctgggattacaggtgtgagccaccacgcccggccttttgcccatttttaattgggttatctgttttcctactattgattgtttgagttctctatatatttgggatattaactccttatcaaatgtatgctttgtaaatatttttttcccattccttaggttatctcttcactccattgattgtttcctttgctgtgcggaaagtttagtttgatgcaatcccattcatctatttttgcttttgttgtctgtgctttgggggttatagccaaaaaataactgcccagaccaatgtcaagaaactttcccttatgtttctttctagtagttttactgtttccagtcttacatttaagtctttattccattttaacttggcttatgtgtatattgtaagataagggtcccatctcattcttttgcatatggatatccaattttccttacaccatttgttgaagaggctgttccttctccattgtgtgttcttggcacctttgtcaaagatcaattggccataaatgtgtggatttacttctggactctattttgtttcattggcctaaatgtctgtttttatgccagtatcatactattttgattactatagctttgcagtatattttgagatcaagtagtgtgatgaccccagctttgttcttttggttcagatttctttggctagggtcttttgtggccccatacaaattttagaattgtattttctatttctgtgaaaaacatcactggaatttcgatagagactgcattgaatctgtagattgctctgagtagcgtggactttataacaataataatgcctccaatccatgaacataggatatttttccatttatttgtttcttctttaattttttatcaatattttacagttttaagtatacagatctttcattttgttggttaaatttatttctaagtatttttggggggatgatactataaatgagattgttttcttgatttctgtttcagttttcttgatttcttgttgttagtgtatagaaaggctacttatttatgtatgttagttttgtattttacagctatactgagcttgtttattagttacaatggaatattagtcaatctttacaaagaaggaaattctgtcatttgcaacaacatggatgaaactggaggacattatattaagtgaaataagccagacacagaaagacaaatattgcataatctcacttatatgtggaatctaaaaagttgaattcatataagcagagtgtagaatggtggttaccagggctgggggcagaggtatggagagatattggtcaaacgatacaaagtttccattaaaatgaataatttctgcaaatctattgtataatatgactatagttaatgtatttgtatacttgaaaattggtaagagagtagatcttaaatgttatcaccaccaaaaaaaagtattgaggtgatgcatatgttaattagcttcatttaatcattttacatgtatacatgtatcaaaacatcatgttgtataccacaaatagatgcaattttgattgatcaattatacctttaaaaagatgggaaggaaaaatgcaaaaatttaatcatctatttttgcaagtagataagaactggctctagtacatcactgggtggaacccaggtttcattattttttaaacctcacccaatatggagccaaggctgagaaccactgagctaccaaagccagaattatgtgggagccttggggatcttggaataatacaaacaaaaaaagtctatgtaaagcatacaaggggaaaggaagagagtaatgaagaaaactatcccctgaaatgcaacacagaacaagatggtcacagctagggtaacaagagccagcttctccagggtcagttatctcttaagacatgcctattcttcatttgggttttagaatgccagggccacggatgcttttactgactgcttttcaaggaatataaagatgcattcttattgaaaataattgcttcctatgtgtagtaattaaacaaataaacccttaccaatactgagagttcctgattttgttctctcaagctttgtattgcttacgggataaagtttattcccaaataacgaataaaaaacaaactagatttccaatcaaatttagaacttgggttgactctcactctcctctcatcacttcaacaagatctgcactgcgtaggggtataggagcagagtcaccaaaaaacaaaacaaaacaaaacaacaacaaaaaagggggaggtggttaagtagaacaggcctgggaaagttatcctctctaaagctcatttcccattctgtaaaattgggagtaattcacctccccacagggttactgtgagaagtaactgagatggcacatgggaaacagatggtacagtacaagacactcaataaaatgtgttcatagtaatacaaaaatatatgtagccatggagaacaactaccctttacagaagaattaaaattctcattatagactcttccattgcttgtcatctgtcttcttaattaggactttttgaaaatgtctccagattcccaatttattcatgttgtgaaagaggattaagagggaacacagctgatctaagccttgaccaagtgtgagggacagagaacaaattaaggatgcctgagtcccacccacctttgcccaaacaccattttttgaacatctcaattgtggtacctagcctaattaacaacacagattccagcagcagtaaatgctcttcctagtaaccttcttaatgttctgtggagtcagattctatatctgggccctaaatccataattttcctatctcccttgtgctatcttaccttaagtgactaccttcacccaatgagtgttgttctaagagccaaatgtggcctctgctgcctcatttctgttcaacctcaaaccacgtcctggttccctttataatcgatagttataacaagccaccggtcacacagaactcccttctaatcagctactccacttggaatgtctcttaagatcaacactcaccagttttaatggtcttcttgctcattcaagacctcaagggtcaggtcaatttaaaagaccattggtataaattgagatgatgttgtcctgattcctacataattaatcataatggtgatcattttcaacaggaagaattcagttatggggacatggtattgcagtagactgggcagagccagaagtagaagttgatgaagatacaatgtcttcagtgaaaatcctatatgtaagaaatcttatgctgtctacctctgaagagatgattgaaaaggaattcaacaatatcaaaccaggtaggacatatatacaagattagtttttcaaaacattctttgtcctggtttagtcttgggagggtgtatgtgtcgaggaatttgtccatttcttctagatttctagtttatttgcatagaggtgtttatagtattctctgacggtagtttgtatttctgtgggatcggtggcgatatcccctttatcattttttattgtgcttatttgattcttctctcttttcttctttattagtcttgctagtggtctatcaattttgttgatcttttaaaaaaaacagctcctggattcactgatttttttgaaggatttttgtgcctctatctcattcagttctgctctgatcttagttatttcttgccttctgctagcttttgaatgtgtttgctcttgcttctctagttcttttaattgtgatgttagggtgtcaattttagatctttcctgctttctcttgtgggcatttagtgctataaatttccctctacacattgctttaaatgtgttccagagattctggtatgttatgtatgtctttgttctcatcggtttcaaagaacatctttatttccaccttcatttcgttgtgtactcagtagtcattcaggagcaggctgttcagtttccttgtagttgagtggtttggggtgagtttcttaatcctgagttctagtttgattgcactgtggtctgagagacagtttgttataatttctgttcttttacatttgctgaggagtgctttacttccaactatgtggtcaattttggaataagtgtgatgtggagctgagaagaatgtatattctgttgatttggggtggagaattctgtagatgtctattaggtccgcttggtgcagagctgagttcaattcctgaatatccttgttaactttctgtctcgttgatctgtctaatgttgacagtggggtgttaaagtctcccattattattgtgtgggagtctaagtctctttgtaggtctctaaggacttgctttatgactctgggtactcctgtattgggtgcatatatatttaggatagttagctcgtcttgttgaattgattcctttaccattatgtaatggccttctttgtctcttttgatctttgttggtttaaagtctcttttatcagagactaggattgcaacccctgcctttttttgttttccatttgcttggtagatcttcctccatccatttactttgagcctatgtgtgtctctgcatgtgagatgggtttcctgaatacagcgcactgatgagtcttgactctttatccaatttgccagtctgtgtcttttaattggagcatttagcccacttaccttttaaggttaatattgttatgtgtaaatttgatcctgtcattatgatgttagctggttattttgctcattagttgatgcagtttcttcctagcctggatggtctttacaatttggcatgtttttgcagtcgctggtactggttattcctttccatgtttagcgcttccttcaggagttcttgtagggcaggcctggtagtgacaaaatctctcagaatttgcttgtctgtaaagtattttatttctcctttgcttatgaagcttagtttggctggatatgaaattctgggttgaaaattattttctttaagaatgttgaatattggcccccactctctcctggcttgtcgagtttctgccgagagatcaaaacattctttaaaagcacctatgcttgagatgacttcccataactttaaatagaaatgcttttatttagttcatctcataattccatgcagacctctcaaactatgttcctaactttggattagaaaatcaagccatgtggtcctacaacatttatcaatattataattgcatacttatctatgtgattatttaatgaatatctattttcttcactaaactataaatttcatgagtttttcccctcttattgtttcctccatcttcagacatttaatctctgggcctggggtacaatgattgctcaataaatatttactgtaatgaagaatgcatgccataaccataggtctgatgcagggtttctcagcctcaccactattgatattttggaacagatcattcttggctgtgggggtctgtccagtggattgtaggatgtttagcagcatcccaggcctgtctctgttagataccagtagcaacctccaagatgtgacaacgaaaaatgccttcagacattgcaaaatatactctacgggtgggcaaaattgcccccagttgagaaccactggcctaatggaatacacttgtgacataagtaaaaaggtgacaatcctaggaaactgatttgagtgaagaggaagggaaaggaatagagggaaggtggaaggggatagttagttgctactcatcttgtcaatacttttcagtatcaattctaagccatcagattgactccccatctctttcttctccatataggagctacaacactgagagaggtagagcatttcaggaaacaatatccaaattaccatttcccaaaaacagttcagaacattaattctacaatcttttggtggatgttgctgataaaagggctccatggtcaaataagcttgtaaacaccagattctgcaaaattaaatgaatttcttcattatggaacttatcaaagtcttttgccttctaacatgctttgaaattctccaagaagcgatgcagaaatcttcatttttcaagtttctgaaacatgactgctcttttttgccatatcattttgtgggattactgtgtctttaaacacactctgggcagtagtggcttagggttatcatagtagtaatatcctttcataactgagaatttcccagtctcctggcaaattatctaggttgttaataagggtgggccatggtgctgtcatgttaaagctgcctaagaatattcacttgcacatactcacatcgtctgtttccaactctgggtgctacttcagatagaacagaatgataagctgatgatttgctctgaaggacattcaggaaggatgtcactgctccattttgaaagattcaccaaagatctttctccagcctcagggactctgccttccctgaaatattagtgtgagggtgtggctccacaccaacagctgtgaatccatgactaagctagaatctgcaaatgatggaccaccttactgtgtgccaagcattgcagtgggcactttcacagcatgtgtcatttattcatcacaaccctataatacaggttgctttattcattccattactttctagataagaaaaacaggacccagagtagttaagtagtttgcccaggttcacacagctagcaggtcgcagatcctggatttgaactcaggtctggtaaagggcatgttcttaaaaactttaatactctcatgccgcctccttattccattcactcactcattcattcaactgtctttccttcagtagctcctacatgccaggcactgtggaaggaacaaacaagtatgatgtgtgcatggaaaaagtttaagaaattccagaaaaatacagctttgacagactactcttggagtgcagagaagacttgtgttttgaagcgacatctaagctgagatctgaatgttgatgcagttgtaggcataggaacattatgtgtgtataaatattgcccactattctcccctcattccatgttgactttctaccttcccagagtgaataatacttcttatttttatctaattaaaacagcacctttttttgccaagggctagaatgagaaactactaaaaggctgcttcctttcctagtccctgattatcagctttcccagtctgcgtattggcatatattaaagagacatgtgtgcccggatcctactaattatcatgaaaataatgaacatcacatatctctctcttgctcactctctcactttctctttcttcttgggactaaagtgtgtccagcgatttcagggcacagtctgcagctggtggcacaactctcagggctatataatgatgtatggcttgttcttcgtggccctctgcaaaaaaaaatatgaatttcagaacgtgaacaactagcagacatctgctgcattaagagggaaaagtcagagctaactagtttatcttggaaagtatgtactgtgccaaggctatacttatctagaaaaatttctggaaacaaacagaaaaatgactctaaaaattccatttaaagtttaggtccctcagaggaacctacatattacctcaaaatatttgaccacgtatcagtttaaccatgatgaacttgaataatattatattgccactgagagacacaaaagataattagaacaaaaattgagacctgaagatggtttgttcttccccactgtagcctaagggtatctgctgaatttccaattatagtcattctacaaaatctgatcttcctacaagagtttagcagtattagaatcacctgaagggcatgttaaatcactgattgcttggcccatcatgggtgggttcaagcatttccatttctattaagtttccaggcaaagtggattctgctggtcagggcaccacacttcgggaaccatgaccctctgataaccttccctttggcatgcaggtgtgtttctcctcaggtttatgtgttgctgacaagttgataagaaacacagcaataattttccaattccttgcatcatgatttcagaggatctttttcttctttgctaatggatgattgagtaataatgatcactcatattctatggactcaccctctgccaaatgaggatcaattattaaacaaactcctatgcactaaagaagaaacataataaaataagccatgtggtgaatactattgtagagaaaaccaacttttatgacacagactctaactttttaacttgtctgttttatgaggctaagtttttaaatatcaaattttccaaaaccagatgcatctggaagttatcgtccaacagtgtaagataatttcagatatctgcaaacagaatttcagaaaatctaaattcattatcatctcaaatgccactaactggtgtaaatgacagaagttgggatgtgaattaactgataatattcccttattccatgtgtatttatagagtagctcctgtgtccaaggcagtaggcaggaaccaggtgaagatgataccaagaagtctctgaagagggtcctgttctcagggagataacagaccagtgagcctaggtggagaatgacattgaaatatgaaccaagcaaagactggaaaggtgaaggtcatcattggccaacgtcttccttctcagttactaaaacaagtcatgggattaagtcatgatcatcaccccatgtgacactgaaaggaatgaagagactatattggatattcagttaattatatgtctcatgcttcatggtctcagagagctttatttatccttagcctggccagataattttgctctgtataaccattgccataagaagtcaagggtcgactgttgaagggagtgaaaagagaagtatcacaatgtctttaaattgtccttatgttacaggccgactatcattccaaaggaagataattcttagtcattgaaccacgtgggagagtaaaaactctaattacctttgttaccttgaatctaagaagataatgaaactttgataaggacagaaaggacttccccctcttcttttccctggcataatgtctcatcctatgttgtctcccggcactctcaaggtacacccaagtgagtgtagggattaaagaagataattggcatgctaaactaatagtggttaaacaaatttccaccttaccttcactctaagcaaaaggtctatttttaaaattgagtaaggaaaaacattttaaaaatataacatccaactatctcggtacaactatctttttcatttttatatgttgccatcatgttcagcagttcccttacataaaaatagatagttggtttgtttgggttttctacacaaataatataaataaactttgcaaatatttgtaaaataccaaatatatcaattaaaatatgaaaacctccttcattcttcccaacttttctccccattcctgaagtaacctctgttaagagtttggtctctatcttttcagttcactttctgtatgagtgtatttatatttgtgtgcacatatatatggttttggattattaaaaacacaaatgctgtaatattgtacttactattttataactttttaaaaattaacaataggtcttgggccaggcacggtggctcatgcctgtaatctcaacattttggcaggccgaggtaggcagatcacctgaggtcgggagttcgagaccagcctgaccaacatggtgaaactccttctctactaaaaatacaaaattagccaggcacatggtgatgcatgcctgtaatcccagctactcgggaggctgaggcagaagaatcgcttgaaccagggaggggtgggttgcggtgagctgagatcacgccattgcactctagcctgggcaacaagagcaaaactccgtctgaaaaaaaaaaataggtctctaatctttccatgtaacaacatacaggtctattttctttatagactccttcgtgttggtataaatgcagcggatttaatcactcatgaaaatagacattaaattgttttatttttgctctttcaacagtagtgtaattgatatccttatgcatgatccttgtacatgtgtgtgagtgtttctgtaagagacattactcaaagtagaatagtagatatttacattgtttccaatctttagctatcatgacagcaaggcaggcaacttatctttgttcccagatgttctttcggaaacaactttctttttcttctttttgttttttatttaaagattattggataaggatatagagaatccagttgtagttcctctctgtgtgcactctaaatctcagttcccttgtcagaactgtagttacctaatatttaaatttttaaaagtaatttaaagttaattacataagacaaaagagataaagacattttgctggggtgaaaagcaaaagtaatcacctaaatatatgtttgtaaaatgatcaataattgagttcaactcaattcaacaagtattttaccatgcaaaatgtactatcttttacattacataaaatgcaaaagaagatggaattcccacccttagacaccttgtaatctaggaagtggaagacacaaatccataacaaattataataagtcagaatataaattttattttaaaaactgcaaaaaaaatcttctgttgaatgttcttgatgagatccttacttaaatatgaggtatccataaaaaaaatcaaatactctttgcagctagatgctaaaatatatcactttcaaggctctcttaattttgttaatggggcaaccttcactctattttacaggtgctgtggagagggtgaagaaaattcgagactatgcttttgtgcacttcagtaaccgagaagatgcagttgaggctatgaaagctttaaatggcaaggtaaggtagaagaaagaaaaaaatatgcagattacatccaagtttggtgtggatattacatccaaaagcacagtcctgcaagcctgtctcactccagccttctctgacatggctgcctcagtccatttaagatgtactaaagagaaatgatggaaaatacaatggaagttggagatgtacctaatatatccaactttacctctatttgttttttataatttggacaagtcactggccctcaatttccatggaaagaggagatttaagtagattatttcaagatgtttttcacaattccagttttctctcattacttcaaagagattatcatcattttagtgcagtattttagaagctgatgaattaaaaacatgtcaagatagctgttattctgccttcatagtttgatcttccacttggaaagcttagagtctctcaccttgccaagagtaggggctggggaagcttctatatcctcactactgagttgtctacagaaagcaatataatatttatatatcaagccaataaacatttaaaaccttctgtagaccacttatcatgctaggatacaaagagaatcaaagatgtttgacaaagagttaaaagttggttggaggaataagacaactacacaaactcgtgcaaagtataggcaagtaaatatcataaagagagataaacgtggtgagcaagttcaaaggaagaagtgggctataaagaataaaacatcttcatggatatgagaagataatatttaagagaagccacgaaaattgggtataatttcaaaaatcaaaatatggaaagacaggaagtcttagaggcacagaatttgagaagagcaagacaagattggagaacaggaaaatgtggagtaacttggctggaacataatgcacgtgggactgtgttacctatcggatctggcatcggtcagggtcccaaaagaaaacaaatagcaaaggaattaattacaaaagtgcgagtggggactagggaaatcaaaaaagagtgtcagaatttggggctcacagcattctggagtcaaaggtacaaggggctggagaattaatagaaatcctgaaaggggagagaattgtgtagagcagatctctttgcaagaagttgctgggtgcagtggctcgcacctgtaatctcagcactttgggaggctgaggtagaaggattgcttgagcccaggagtgcaagaccagcctggacaagatggcaagacctcatctctacaaaaaattagccgagcatggtagcatggactactacagtagtcccagctactccgggggctgaggtgggaggatctcttgatcccaggaggctgaggctgcagtgatctatgattgtgcctttgcactccagcctgggcaacagagtgagaccccaccattaaggaaagaaagaaaagagaaaagaaagaagcagtgattttggtcaaaggacacagtcagcccaaggtgacctctcaaagacgggtccagcagaataaatgccccgaattcactgttgtctcttctccaacctcctaccagtgttccccattggctaaatgcaatcacaatctagaaggctaaagaggaccccatgtagaacagcttcccagagcagagagcaggctggagctggtaggtggtgaattccataggcagtggatccagatggcaaagggaggatctcttagcctaggcccttcagacactgtggagcctcttgcatattagctcctcaggggcccactcaaactcaaaaagtttgaatactggagggtggggtggagaaagtattcactctgaaatacaaaaaagaaaactgcaaagaaaaactaatgcataaatacatgtctataaagaaaagcattatttcatcttcattgatgatcacatttagtatcataaaagtgttattacttggaaaacaatttgtaagttagattttctctatatggaaacattcctgagtatacatgatgattgctgggaaggaacagccatcacaaattaaatgccaaaaattacgttggaataattacaattgaaacatattcaaattcccaatttttgcagctagatatttttagagtaattatttacacatattatttagttaaatatatatttaatattatatttgactataatgtggtatggaaaaatcaaaagtaagttttcctaggaactatgaaggtttttgagcatatatagagatatttatatttaatttgttagatgaagtgagatcttgaaattttttctacgataattatcacatttacaatttaggaaaattagcctagtaagaactgactttttaatacaggtgagagaaacatgtcatagaaactagttataaagctattatagtcaaccaaatgggacaaaataaagtcttgaacttaggtaggaatggaatgtaggagacagccatatgcagaacagtaagggataaattcaaaggacgcaggaactgattggagatggggtgaaaaagaaggacaagtcattataagcttccaatatggtaggttgttatgaaagtcaggaagggaagcctattcagagagaaaaatgatgaattgagcttcagaatgattattcatttattctttttgtaagtctatgaagtgaaagaaattttactaagcttgggctatacagattaataagatattgtcctagctctggaaatatctaaaccaaataactaaaatacaatattgaatttaaaatgcttttggatgggacaaaggaaggcagaacaaaatttgttacaagatatctgaatttgccgcaaagaatcattccatgcaaaacatctcttgatatggaagtgcctataagcagagacctatttgggtctctcatcttcctcaagactggactgaagatataaagaatcttatacccaagttcctcccctattttcagtatgccaggtctactttttctaattacatcaacatcaatataagattgttgaatatcagctggattctgtctgtctctcagagtattccatctaacttatttgacactggctcctgctactaaaaagtaaggtaaatacataatactggggacaaattgaccaagaggctgaacaaaatggtcaggatgaccatgaagcacaagctttgtccagcagacttcaataatccaggaaccactgagtcactgttgaaatcagagggtaagggtgcacactcagctgctgtgaatgggaatttctgtagaaacactttacataaaagtgttctttatataaaacagaggatcttcacattcttattcaaaaacaaagccacaaatgctttcagacaatactttgttcctttgataataaatacatcaatctatcaagaatttcttgagagccatcattgtttattgtgtaggcaagcaatacatagaagttagtagcacacgtagatctacatacatccattatctttgacatctggagctgtttgcagtctcagaatttgtgtcagaattcttaaggacaggttttaagcagtgcatgatctgagaacctcaatactactctcactcccaactactcctgacccctgtcatccctgtttattagctcccaactgttcagcagatcaaaaggtggcttcacaaggaagaacctgtggcaggcaatgcaacagcctcccagattcctcttcctttggtagagccacagtatgtctctcttctccagtgaaaacttacttgtgtggctgctctaaggagcaagacagttagcgggctatcagctggaagggcctgatttggctgcccatgctaaatgctaataattgataagatttctttattggtttcctttcaccttcatcttattgtgcaaggtaatggagattttataccatccataacaaacctaaattgtcttgtagttatataattatataaatatgtaatttaaaacacatatatgacctaataaaaatattacataactttctgtctcagtgactgtaaccagattgtaaatttataatgaatatgtcaatggacactggccaatatgtcactgtcaaacctgttggcatcttatgcaacttaccaacaaaatggctcagaaaaagttcaactggcatacaaaacaaacctataaaatgacaagatacaatcaataaacctacagcaaatagtctaacacttagaaggagtcatgttattagggctcagccacacttggttatcttcatatcatctcgcgttgatatttctttgctctctacaacaggtgctggatggttcccccattgaagtcaccctagcaaaaccagtggacaaggacagttatgttaggtatacccgaggcacaggtggaaggggcaccatgctgcaaggagagtatacctactctttgggccaagtttatgatcccaccacaacctaccttggagctcctgtcttctatgccccccagacctatgcagcaattcccagtcttcatttcccagccaccaaaggacatctcagcaacagagccattatccgagccccttctgttagaggtaacaccagtcagatcttgctttagagtaatcccaagatttcaaatgactcaaattgcagtagccttagatacattggatgggtccacttggagaaaagcactgaaaaattgtttattaaaaatcaactattatatcaatagttttcctttaataggaatctttttcacattacaactggttattgaaaaaacgtcatcagaacattgtgcaaatgagcctagtgcacagattcaaactttgcatgggcccataaaatttgcacaaactaaaacctagttctataattgttagcttcttctatatcacttgtccaccactgtgtagtcatctgtggctcattacaacacttctggaatagactagtatagtccagtggaaaccaattacctcaaatagcaaaaccctttcaaggcatgtgtgtatttgatggtgactcagtggcattgtcttaatatgtaaatcacaaattattataaatataatctaacagacatatgagatgttctcatctgacctcctccaggaaatatgttatactttcttagaagccaaataattaaggacattactatatttctttaatacatggagaactgaacaacaaatagaaaaaaaaccctcaatcagactgctaataaaacaataaatatttcacatttgaatcctgtaagaattctaaatgtttgagcagttttccaagctccatttcttcaagaaagctagaagataaagaaagcctttattctatttccaagaaactagcttgtccctgtctttttggctccttcagatgagtttttctaggatgtggctaatgccaactgaatcccagttctatcctccaaatacgacatgaatctgggcaattcgataaactgtgcatttaagtctattcttgggtttgacttagaagcaaaaaggcacatctagattactgtggaaacaattatcttactatgttttactacttttcagtataactaaattgatcatattaggagctctagtcttcaacagccttaaaagtttcatactcataattacttgccccaaaattgtaatgcaaatgaaataatcaagatttcctttaaggtaacttgtctatgtgataataaattatatcattgcaacattatgcatttagaatataaaatcacctcattaaccaatccatacattaaatacaaattgtcaaccagttatgatgtcgaaaaaaaacaaactccactataagggaaaaaatgcctatcttataacaagaaaggaatcaatgaatgatattcatggatgaagggtaatcaatttaattttttatctctatgccaccaatataaaatcaaagtagtaacatggcaaaacaaaaatcaaaaggaatctatgcatgaaactcaatggaaatttaagcctcacattgaccgaacatcagagagcatggtgaacctaaaagcatgctttctaaatgtttgttttacaataagtcattggaagtgggataattagggaagccagaatttatttctgatatactgatcagtgacagtctctatgcaagactgatttcaaaataagtggtcgcattatgttggaatgttctaataaatacattcccagtctttattagtctgtataaaatatcaaggttacctaaagacaaaaattgcaaattttctatcccaaaatttttaagttgtttgtgtacttatcaatagtattgtatttatcaatggcattgttttcttctgaaggtagtttttcaaaaaagagagacacctatgttaaaccttaccttatgttgagtttcattacattagaatcctagagtctcccacccatcttcagggcaataatcaacgtgaattgggctaacttttccattacagtaagattcagggtgattttctggtggtttgattcagtttaatgccttcttacgttcctagaaatttacatgaatgtacctgtaggggctgcgggagtgagaggactgggcggccgtggctatttggcatacacaggcctgggtcgaggataccaggtcaaaggagacaaaagagaagacaaactctatgacattttacctgggatggagctcaccccaatgaatcctgtcacattaaaaccccaaggaattaaactcgctccccaggtaggtaatgttattccagaaatagtttctttaagttgtgccaatgagcatcattcacttttatcaaatgatttgacttgatccctactcaagctatgggtttttttcttttaatatataaagcatccaatgaatgtcacagtctttgaaggaatgctgggcatctggtgccattattctattataggtcctttctcttttttaaaaaatcataaagtctaacaccttgaacaatattccactgaaatgcaaagaatctgagagctattaagaattatcaacccatacccgaaatgaggcaaagaccccatctcagaataaatgcctattgtatgtgagccaggttggctggcaaatgaagagtattatggcagtgccctgaatagaccatttaggacatcttttcttggaaatactgtatctaagagaaaactccaccatgatatctgggtgaattattaactatgagatgtcatcagtcataccaaattaactcttcctccatagttatattttggcagtggcattacagagagtttttactgtgttcatttaacagttctaagtttccacacttcaacattaacctgtggtttttattattggaaaattaaaagctataaaagtttcaatatggaaactgactcccggggttaaaaataaaacaaaacagagtctatgctaaccctgagactgttccctgtgggagtgtgataggtgctgatctctctgaaatacattttggctgaataatcagagttacagtttgtgtatcttgaaggaaacttccctattcccatgacaaaggtgggcttatagcctcacatttatggaagtagcccttataatttcaatccagaagaaaactggttttctaaaagctcatcttgggtaattttttaatgggatttctggctttcttttatgcacaaattagcttatctaaggtttctgctagccccacccagggaattttaaaaaaaatgcctatcagactgctaaacagcaatcatttgttcacataagatttagtaaacttctcttatgcaccatgtagtgcgctaggtgctggagatgtagaagtgaatataacaaaatccttatcatgaaaaattagatggcaatccagtgggaaaaaactgacacgtaaacatataatgaaatctggaagtgatatgatgaagtttgttcagaaaatataaaagccactcatccagtttctcttaggttaaaacccaaaacaaaaaacctgattaaagtactggaatacttagaaatattttaagaaaataaaaaataatagtcgcaaattctgtataataatagttccgagagctgcaatagtatgaaaagagatctcttcattttacattaaactcaagttagaaattcatcatgtcttttattagtatgtgagtaacaatctctatcaattaataaaagttctttaatttgtgtctgaccttctacaactatggcttagaaatgaagaccatgccaccaccaaccaacacagatggggcctaaacaatcccaaagtagagttgtgaacttccagccagaagcagagagacaagccctttcctgcaaagtcagcagtgagagctggtgttccttccgcccaggtttgctcacctggcataaacaagcaggcataatatctaccagcaaaagctcctttattattgcccaagaactgtagagaatgctctgaggattacaagagatcctacacaacaagagtattaaaaagaaaatatcaatagaaaaaacagtaacaatctacaaattaataaactggaaaatatcatgctattgcagctgggttagttagcataaatgaggtaagaaacctcagagacacatcctgagacattgttctgtcttacagagttattttctcaggcttatgccctgctttccacagtctactgaatctgctgactaacgagagcatcaggacctctggacaaccaaaaatggcttcagaataaagaaactatgaatctttagaaaatattcagtttggagtaattacagaagaaaaaacatgcaggaggatgctaaaaagcatattgtcattaagcaaaaaaacaaacaaacaaacaaaaaaggcagaaatagtgtcttaccaaacaaataaaaacattgcaactgtcaacacccatctaggaacactcgataaataccaagcattaaactttttaattctatttttatgaaattgtgagattaaaaccctatgacaaatgtcacgaaataaaaatatattcttaggctatcatctctaaaccattataaacacagagattgagagtaataatgagtaaatctatacaaatctatacacacagtgttggagcaacaatttttacaaaaacaaatattccaaacaaatcataaaattaagcttttggtaaaaagttacagctaatagactaaactaaaaacatattacagtaaatggtgtgaaagtgattttatgaactaatagacctaacattttaaatggagttcaaattgagttcatcttaaattttatgagtcattaaatcatgtggcattttatgtcttcagatactagcttgaactctgaaaattagagtttaaaaacctgaaaacttgggaaataatgtgaatgtgtcatttgagggggaaataactttttttattcttgtagatattagaagagatttgtcagaaaaataactggggacagccagtgtaccagctgcactctgctattggacaagaccaaagacagctattcttgtacaaaataactattcctgctctagccagccagaatcctgcaatgtgcgtaacttctccaaattccttaccattttagagcaggatacacttataaattaaatgcatttagtcaccatctaaacaagatactgaggtctactacctactcatttgtcattcaatgcaaatattgagatgtgtagttgggatgtgccagccatgaaaccagcatataaaggcccccaaatttcatatacacaatgaaggttgtgtagtatcaccagccagtggataacagtcagtagtgtaaaatgtaatagaaataaaaactatagcccattcttggctggatcacttgaggtcgggagtttgagaccaacctggccaacatggtgaaaccccgtctctactaaaaatacaaaaattagccggatgtagtagccggtacaggcaggcgcctgtagtcatagttactcagcaggcgcctgtagtcatagttactcagcaggctgaggcaggagaatcacttgaacctgagaggtggagtttgcttagattgtaccactgcactccaacctgggcaatagagtgagactccatttcaaacaaacaaagctatagcccattctttcatctgtgcagtgtttaggttgaagcatatgaaactaccattttatacataataagacaatggcccatcatttaattttggaccaaagatatgacctctctgttagtggatatcacctcctgaattttaccttgagcctgcatttctatatattttaggaacaatttgtttcacttgtgctttagaaaagccatgtagtgaattacttgtatgtcccaggaatgtcatcaaccactgatgttctagatcaatttgtaaatatttctacatgcaacctatatttagaagctttaatagcaaacagaatgaaaaacacccactcaaaataccaccatccagtcatcttacaaagctgatagtgccttaagttttcacctgactcagttgtaccagtttaaaaatgcctaccctgactgacttgctttccagccaccctttcacacctccaaagctgagtgcctttgtggatgaagcaaagacgtatgcagccgaatacaccctgcagaccctgggcatccccactgatggaggcgatggcaccatggctactgctgctgctgctgctactgctttcccaggtatgggaaaattgtgccagaaatggcgccctgcagagagtatttgggaaacccttttttggtaccattgaatcacagaaatgtttgtgtaccctacttacagcaatctgacttgcacaaccttgggaccaaaaagagtttcccaaatgagtgactcattaaaacacgaggcaagaaaatcaagctgggcagcagagtgggttctggtctcggctcagctttaaactgctgtgtgatcttgaatgagtcagtctctctgagtctggtttgttttggtttttacttttagtacgagagtttgaaactacctatagatatatgatgtttattggagatcttactgtgtgctaggcactcacttaaatggtcaggaagcgttataatatcttagtaatactcatgataccaccaagagttaggttccatcagaaagtctggtatacaaatgaagagcccatatacagaaagattaagtaatttggccactggcaattagcttttcagcaatcttttctccctctccagtcttcaatctacttttattaatttgatttcaaaaaagattttttaaagtcaaaaattttgtgagagtgagagacagacagagatcctttcaatatgtgaataatcaggactcaactcaatttatttgttctataattttggtctggggcatatgctgattgcttaaagcaaagcctatccgtggatcaacaatgccttcagaggtcacattttaaaagatttgtccccatttgttatgtacccacaacaaatggtaataattaaaactgaatcctttagagattgaaggggaccaggcaagaaagttgtttaggaaattttttctcagctgaaaattttttatcctttttttttttagtaaaagattatttttattaaagaaatctgtcattttttacttctcattgtgtcagcgtgtgccacataagtccaaattagagctctaagtagacctagaaacatgatattcttattgattaattaattcatatattggccatgcgtaggtgtgaaaaattttatattcctcacacccactgcacctggggctccttctcagtagtgttttttggttttggggttttttttctcaaaagttttgaatatatcctccattctggccagtggggctaacattaatgatgtttcacatctgaaagtgacctcaattctggctttgtttctctgtaagaacatggagatgctctgtggtgagtccctcggaggttaccaaggtaacagctgacattgcagagggcaaggtccttcctgggcagtgtggggccagcagccagtgcttgctttccacataacagggtgtttacatgtccatttactcaggatgcctgccagggcttctcagttcctagagaaagttgagcccctgtcttacagaaagctgcaccccaaagacacctaacaaatgtactattaaactaatgagccactgtcagaattttaaagtttgctttcacattggaaaggatttgcttttacccaagaatgttttatcttattctcacttccactggcttctgtgagaagttaaaaatatttagggcatgagtgttgtcttgggaatgcctaagaatctgagagcctctcttataactttgctgtggagtcaagcaaagaataataggctccaagggaagaatcagtttgaaacccaaacaccctggcacttaacaaattgaccaagttgaaaaagaaaattcaaaatccctaaaggtaaaatctgtgccacaaacacatgcatgcatgtgtgcacacactcacacagttacattactcacttagtttagaaatactgtttccaaaaccatgaaagtaactgttaacacatgtctcctatccttggaaaatggaccaaacttcactagtgcattaatcagcattactgaccaaaatgaatgcaccaagagtggattcaatttttgttcaaaataatccaaccagcttcctgccaccactttctgagaagccactagaaaaaaatcaatgtgtctggggttgggcaggcttctactgcacataaactaaatggggtttaaaacaacaatgaaaaataggtgcccatttgtacttaagtatacttctctctttttcttgctatcttttacaatttctggataatctcagaaatactcaggttttaaactaaaacaatacagagaaataatgctttagatagtagctaatatgctctcttcttcaaaagtgaatgctcatccactagacacaatgctaacatgaaagcgcatagattatttttttaattctcagatactttggttctctgaggcggagaaccagcatcaaggcctctgaatacaacaagcacaggcttgatgaggggcttgggaggtaagaaggtgcttttgccattcatagttacatgtaaatatggacaggttaggcatttgcttttaaggtattatggaaaagcacacttaacctttacagtatttctcctactgatctcccagtctttacacatcttcttgggtaaaggcaaatgttttaaatgtttcatttaggcaggtgtggggttacaggagaaccagtcccacagatccccacctccccacataccatgcccagccagcccctcagtcaccttttctcatttataaatttggagaccctcaaaacgctaaaaatcacaagctttaatcaactcagtatcctgttaagtttctctttcttggaccctatcccaagcatctttaattctcttaagtataaagtgattctgacacaggtggttgaccccactttagggaacattgtcctaggactgtttaaatatcattgaaaatcactgagtccacatattgtgtccttaaggacactttttaaaacatatatattaacttttagctttaagcaatatgttaaatgttcgtatttctaaaagaagacaaataagccaaaagggagcaaatgtgaattcctttcatcaagttttctctgcctcttcctccaggatatgctgtccctaatgcaactgcacccgtgtctgcagcccagctcaagcaagcggtaacccttggacaagacttagcagcatatacaacctatgaggtctacccaacttttgcagtgactgcccgaggggatggatatggcaccttctgaagatgcttttttaaatttaagaataagacacacaaaactctattaaaaaaaaaaaagaaataaacctctaactcggtccccaatgatcataaataatatgtttcctaaagaaatgcctttccagagactgtatagcttataccaattatagaatcatgaagtaagaacgttattctgaaatgaaatcaaatgttaaagagccttttgtttaacagaaagctggagagccactgccccaaaagttgttcttccagtcttcctgagtgtttggggagtatgtttacaaatcctgccagggtgaagagagatcttgtagctggcaagatttgggagggatggagcagacctgggcagtcagtaataatcaagggaagccaagtaaactccagagtcttacctctggctcacagctgcttttgtgatacacaaagtcacaaagaggaaaagggaagggaggaggttgcttttcaaaatgcattcaccatggctcactgtgtggggcatgattgcaggcctgagctcatgaaagactgtccaagattttgtggtttcaacctctcttcagcctgaaaattccatccagtcgatttgtcacagatcaggatcttcctgtcccctgcagcccatggggacttcctagaaatagatatgatatgttgtagatgtggttaaaacaaatccaggccccattgtttattcagctcattagtccacacctatttttccctagaactcctaccttttccaataatcatgccagcatgaaatgaccttttctactttaaaattttttgacttgttcatattgcttgattttcatcatctcagactcatttttctcctacctaatatcataaaaaagtaaatcagatatacagaatatcatttatgatacttcttcctttcagcaaattgactgaaaagtcattttaaagagagaagtgcagatttgctctcatttctttgatattgttgcagtatccaccccaccatctagcaatcagagatgcttatattaacttgggattagctttgcattatctaacccatttattttaaatctgccaggaaatcctctaactttccttcctttttgtttcagtaagtatcaggcagcttcaccatacctgagtccttttgtcttgaagctgccacagaaaaatcttacagcaatcattgctgattagaaactgtttcagacaatcagcatgggtgttatttaccaaattccccccagagtcctaggcctcttctccagaaatatctgatgatgaagtgaggggagggcaacggtgctacaaaacacggaacagaggtaaagagaaggcactactttcttgccatacttgtaaatgattgctttgttcaaacataaataatcttaagtccaacaccaaatacctgttactcctacatcaatctcattagtggtttaagacacagtactagaattttcattttttaaaatcccttggcccttaaaaaaatttacagaacacaaaggaaaacataaacacaaagacatggaaaattttgtcaactccttaatggaattctgtgatcaaaaagcaggccagattctaatcaaaatcaggtaaattttaatcacaatcagaagtacttgtaacatttcagttgtcctaactccaatgagataacaaagcctccaaggctacagctgaaactctgaaaggccctgtgctttctactttacatttagcgtctaatatttcctaggacagtagttcccaaagtaggctgtacattagaatctcctggagagctttttaaatgctaatgccaataacatatctcataaaatttacactagaatttccttggatgggtgcctggcatcagtattttttaatgatccctaggtgatttcaatgtgcacctaggtttgggaattgctatattcttgtatttatactctatctctttatttcttttcctttttatttcacttttagtaaatagaaaaatgcaattaaaaaatcatctaagttaagacaaatagccaaatcatatttatcccataaatagtatgaatttaaatgtaacttacgtaggcttcaaactaactctaaaaatgagaaagtaaaacaatcttgtcctaaaacaattcttagatccaaatttacactcccctacagatctgcttttattctcaaagacagtgattacattgacataacaaatgtccattggggcaggcaaatttctactgagtctaaaaagcattccaaattctaggagcaaaagttctgattttccttaaaatctttgcaaataaacagcaaagggtattctggatgtcttgtcaatgcaaattaggtttgatttagagaagttttaaatttaatcgcaatgaaaagattagttaatacatgaatctttttatgagaaaaaatcattattctggataaagtcataattgtttgtttacaatattaagagcagacacctaaacatacaataataccaaagtattgagaaaattaccactggaacaccatctcaagttatcacaccatttacctctttagagccagttttgtataagtaggtagaagtggttttggtttttttgctaaaattttactttgttttagtaatatctagggaatactttgaattccattaaaataccttcatagaaagtgttaatgtgtctaccatgccatatgtttatttttactttccccaagttaggtagatagagcaagtcctgctaccataagtttaaaattattaagacaacgggcttttttaaattgctggtagaaccaaaggctaattttgcataggtttatatttttagacttaagaaattagcacctatataataccttcataccttctgcatcactatcaatttttctgtaatttcttatttgaatgcttcctaattctttacagaaaaaattaaaatttgtcacccaaccaggtgataataacaaaacaataatttaggaacatccagtaaatttgaattagttctctaaattttggtaattaatagcttattgataattaaaatcagcctaatgttgcttaagtaggaggaaattgatcagcaatgggattgggtatctattttctttcctataaaagtgctgcctcttctcaaattgctttatttctctcccactgcattagtatatctagaacagcaccttcaactgcaacaaaatataattctcctagatgactaggatatcctccaatctggtatcatgccaaacaacactatgtttggggaacagcacccgaaatgctgctaaaaagctacctcatgtcaagaaaatatggatttcaaaaaatatctggcacagtggcccatgcctataaccccagtactttgggaggccaaggcaggaggattgctggaggccaggagttcaagaccagcctgggcaacatggagagactccgcctctacaaaaaaaaaaaaaacaaatttaatttaattggccagttgtggtggcgtgggcctgtggtcccagctacttgggaagctaaggcaggaggattgctggaggcctggagggcaaggctgcagtgagccatgttcacatcgctgcactccagcctgggtgacagagcaagaccctgtctccaaaaaagcaaaaataaatatctaacaaaaaggaacctgtttccaaaccacagtttcttaactttttcatgatggaatagaaattaaactataagtatatactgataatagctgtgcaggaaattggtttttattttacttaaaagtattttttagaaaaaatgttattctgattgcatagattagcaaaatattgcatcaatataatgttccatttttattaatttttgttatattctaaatgaccgttttataattctgtcttggacaactgggccacagttagaatccacatagacagggaggttcttaggtgtgccatttgtagaataaagggcaatcaagttaatattcggggtaaattttcattgcattctaacctatgaagtttgataaagtatattattattgaacaactctaacactaagatttcctaaaagtaacttacaacaaaaaatgaactatgtgctcaatattcacataaagtcatgattattctcttcttgcattcatataaaagttcccagggggaagaaaatatatctaaaaattagactagaaacttggcactttttatgtattgtagatacactttcatatattgtaaatacatctgttaaagataaaactaagtttctaacatagcactacaaggaaactttggataaatatttttaaaaagaaaatgttaataggataacttaagattttgtatatacttattggaagacaggaagaaactaggcagtaaatattcctacctgacgtgcaaccattgtcaatcaggttttgttaaactgtcaaatttgattttgaaattttagtttgcccaaataatatcttgaaaatgctctgaattttactagaacatattcatagaaagtgtaatgtatctaccaggctctatgtttgtgtttgccaacattagaaaaacaaagtaagttgcgctataatgagtgaaactaaggaaaatgagatttttctgactcccattccctctcttaaaaagtaaaatatttaagctttttttattaaaggaaaaatgggtacagaatactctattgccctccaaatgtccaacatttttaaagccatgttgtccaacaatgtttatacagaaaatagaatgcataggtttaatatagatagcaatgtatatgtaggctcatttgtgggagattgcaaactttatgtgcccccagttctccttggttttgtaggaaagctaaaagaaaaccttgtactctcactaataatgaaattcatagccaatttagaaagggaaaataacctgaaaaccaaaatacctcattcatctgaagatgcctggaataaaatatggaattcttctgtacctacctcccccttacagacttttctcctattttgagagagaaattcaagaagaagaatacagcttgttctctttccacaaatgatactttgtctaagggaataggagataccaagaaacagtatctttaaaaaagtaagctgcccacaggccttcttggagaatgaagaatatcagccccattcagggtttaccaatgtctctctgaggtgacaatacattctggttttccaggacagtcctggcttaggtctgctgtccttctataaatattaacaatgatccctcttatttccaaacgtgtcccagtttagacaacaaattatacagtgtctgcccccaacacacacacacacaccccaccatctttatgggttgttgttgttttcttgttgtttttaattttgtagaattggctttgtcttctccaaagcagataacagcttaaactaaagagcctagctacttctttctgactgagcagggtcatagtaaacagagaagaatggatcacttcctcctctgaaatctccatcaatccactttctgtccaactatctctctacccagcctatttttggtcctagttttcctctcataacccctgggctgtgagtcctgagttctgccttcatatggcagagcaagctagtcattattatgccaggttaggtggtggggcaagacaaaagatagctcttggggacacttgcttggtctgtagagatccacagggcactagtagggtattagagcatttattggtttctttcttccttgagttgagttacttctatcccaaaacagacaggtcttacgttaaccttcaccttaaaaaggatattgccttttttccttctgcttcacaaatttgctccaacactaattcccttgcatttgatttcatcattatgattgttcatcaaccattcttcacgaaaaatcctcacatcacgtcatataaaggttttattatttttaatggaaaataacattgagagattattttcctttttcataaaatttccctttttccctccctctattctcttcactaacattggaaattaaacaagttttacaggctctattgttattaaccctcaaatctattcttcagagaatatatgatcagcctgtgtttactatgcacctactcttataccagaaactgtgaatacaaatacatatatttcctacttctctctttaattggggcacacagtcccatggtccattctgcatttttcaaaattgaatataattgcaacacctgataggttttgccacttagaattctgaaaataaatcaactctgttctaaaccttaagtttccactattattgccaaactctggactgatacaataagccttttaaaaggtgaatttaacacttcacctattatggttatttaagtaaaatgagttttcaaatatttcccccaggagaaatataaaggactatgcaaaaatagaaggagaaaaaggttagttagaggtccagaaggagaattaaaacatggaattgataaattcacctactgatttatgatgttaatgagcaggtacctatacattttatttacttgaaaacattttggccattaactatttctgttttctaaatacatttgtgtttataaaattttaaaatctatatcacaagacttagacatgcctttctttgcaataaagtatatatacctttttacaaatgtaaattttattttgaaacttggcattttctttataattcctaagatatctttgggcacacagactacagataaatctatttaaatcaattcatcattatcactggtttaaatatttaatgtaaccattggaatttttattcttattccttcagagaattctccttttcttctatgtttctgactgcaatacagcccgatattgttattccctatatcttaatgaaaagcaagctgtcagcaaaatgcatttatagtttccagatgtttaacaatctctttcttcttttcttttttgttattatttttatttatgtgaagttagttagctgaggtaccaaaaaagagacaaagacagtattaaaaagtgtgcataaagcacttatttttgtactttagtacaatgttttatagaactttgtgatctatctaaatatattaagtaaaattacaccattcacttgttgggaaaataatctttggtttggaagatattaacataatgggcatcttagaatcataaatcacatgaaatgagagacaatgcaatattgtataattcctggatgatgcaattgttttaattgaattttcaagtgccattataaagttttaaaaattatcaa

SEQ ID NO: 46 - RNA, 9221 nt, Homo sapiens
  source 1..9221: mol_type = mRNA
ataatcaaggaaaccttttccgggtggggatctctgaaattactcagataacagtgctgtgccaaaaacctgtggattttctctacaaaaattattgagcaaccctaattaacctgattttttgctgataatcactctcaatggaatcaaatcacaaatccggggatggattgagcggcactcagaaggaagcagccctccgcgcactggtccagcgcacaggatatagcttggtccaggaaaatggacaaagaaaatatggtggccctccacctggttgggatgctgcaccccctgaaaggggctgtgaaatttttattggaaaacttccccgagacctttttgaggatgagcttataccattatgtgaaaaaatcggtaaaatttatgaaatgagaatgatgatggattttaatggcaacaatagaggatatgcatttgtaacattttcaaataaagtggaagccaagaatgcaatcaagcaacttaataattatgaaattagaaatgggcgcctcttaggggtttgtgccagtgtggacaactgccgattatttgttgggggcatcccaaaaaccaaaaagagagaagaaatcttatcggagatgaaaaaggttactgaaggtgttgtcgatgtcatcgtctacccaagcgctgcagataaaaccaaaaaccgaggctttgccttcgtggagtatgagagtcatcgagcagctgccatggcgaggaggaaactgctaccaggaagaattcagttatggggacatggtattgcagtagactgggcagagccagaagtagaagttgatgaagatacaatgtcttcagtgaaaatcctatatgtaagaaatcttatgctgtctacctctgaagagatgattgaaaaggaattcaacaatatcaaaccaggtgctgtggagagggtgaagaaaattcgagactatgcttttgtgcacttcagtaaccgagaagatgcagttgaggctatgaaagctttaaatggcaaggtgctggatggttcccccattgaagtcaccctagcaaaaccagtggacaaggacagttatgttaggtatacccgaggcacaggtggaaggggcaccatgctgcaaggagagtatacctactctttgggccaagtttatgatcccaccacaacctaccttggagctcctgtcttctatgccccccagacctatgcagcaattcccagtcttcatttcccagccaccaaaggacatctcagcaacagagccattatccgagccccttctgttagaggggctgcgggagtgagaggactgggcggccgtggctatttggcatacacaggcctgggtcgaggataccaggtcaaaggagacaaaagagaagacaaactctatgacattttacctgggatggagctcaccccaatgaatcctgtcacattaaaaccccaaggaattaaactcgctccccagatattagaagagatttgtcagaaaaataactggggacagccagtgtaccagctgcactctgctattggacaagaccaaagacagctattcttgtacaaaataactattcctgctctagccagccagaatcctgcaatccaccctttcacacctccaaagctgagtgcctttgtggatgaagcaaagacgtatgcagccgaatacaccctgcagaccctgggcatccccactgatggaggcgatggcaccatggctactgctgctgctgctgctactgctttcccaggatatgctgtccctaatgcaactgcacccgtgtctgcagcccagctcaagcaagcggtaacccttggacaagacttagcagcatatacaacctatgaggtctacccaacttttgcagtgactgcccgaggggatggatatggcaccttctgaagatgcttttttaaatttaagaataagacacacaaaactctattaaaaaaaaaaaagaaataaacctctaactcggtccccaatgatcataaataatatgtttcctaaagaaatgcctttccagagactgtatagcttataccaattatagaatcatgaagtaagaacgttattctgaaatgaaatcaaatgttaaagagccttttgtttaacagaaagctggagagccactgccccaaaagttgttcttccagtcttcctgagtgtttggggagtatgtttacaaatcctgccagggtgaagagagatcttgtagctggcaagatttgggagggatggagcagacctgggcagtcagtaataatcaagggaagccaagtaaactccagagtcttacctctggctcacagctgcttttgtgatacacaaagtcacaaagaggaaaagggaagggaggaggttgcttttcaaaatgcattcaccatggctcactgtgtggggcatgattgcaggcctgagctcatgaaagactgtccaagattttgtggtttcaacctctcttcagcctgaaaattccatccagtcgatttgtcacagatcaggatcttcctgtcccctgcagcccatggggacttcctagaaatagatatgatatgttgtagatgtggttaaaacaaatccaggccccattgtttattcagctcattagtccacacctatttttccctagaactcctaccttttccaataatcatgccagcatgaaatgaccttttctactttaaaattttttgacttgttcatattgcttgattttcatcatctcagactcatttttctcctacctaatatcataaaaaagtaaatcagatatacagaatatcatttatgatacttcttcctttcagcaaattgactgaaaagtcattttaaagagagaagtgcagatttgctctcatttctttgatattgttgcagtatccaccccaccatctagcaatcagagatgcttatattaacttgggattagctttgcattatctaacccatttattttaaatctgccaggaaatcctctaactttccttcctttttgtttcagtaagtatcaggcagcttcaccatacctgagtccttttgtcttgaagctgccacagaaaaatcttacagcaatcattgctgattagaaactgtttcagacaatcagcatgggtgttatttaccaaattccccccagagtcctaggcctcttctccagaaatatctgatgatgaagtgaggggagggcaacggtgctacaaaacacggaacagaggtaaagagaaggcactactttcttgccatacttgtaaatgattgctttgttcaaacataaataatcttaagtccaacaccaaatacctgttactcctacatcaatctcattagtggtttaagacacagtactagaattttcattttttaaaatcccttggcccttaaaaaaatttacagaacacaaaggaaaacataaacacaaagacatggaaaattttgtcaactccttaatggaattctgtgatcaaaaagcaggccagattctaatcaaaatcaggtaaattttaatcacaatcagaagtacttgtaacatttcagttgtcctaactccaatgagataacaaagcctccaaggctacagctgaaactctgaaaggccctgtgctttctactttacatttagcgtctaatatttcctaggacagtagttcccaaagtaggctgtacattagaatctcctggagagctttttaaatgctaatgccaataacatatctcataaaatttacactagaatttccttggatgggtgcctggcatcagtattttttaatgatccctaggtgatttcaatgtgcacctaggtttgggaattgctatattcttgtatttatactctatctctttatttcttttcctttttatttcacttttagtaaatagaaaaatgcaattaaaaaatcatctaagttaagacaaatagccaaatcatatttatcccataaatagtatgaatttaaatgtaacttacgtaggcttcaaactaactctaaaaatgagaaagtaaaacaatcttgtcctaaaacaattcttagatccaaatttacactcccctacagatctgcttttattctcaaagacagtgattacattgacataacaaatgtccattggggcaggcaaatttctactgagtctaaaaagcattccaaattctaggagcaaaagttctgattttccttaaaatctttgcaaataaacagcaaagggtattctggatgtcttgtcaatgcaaattaggtttgatttagagaagttttaaatttaatcgcaatgaaaagattagttaatacatgaatctttttatgagaaaaaatcattattctggataaagtcataattgtttgtttacaatattaagagcagacacctaaacatacaataataccaaagtattgagaaaattaccactggaacaccatctcaagttatcacaccatttacctctttagagccagttttgtataagtaggtagaagtggttttggtttttttgctaaaattttactttgttttagtaatatctagggaatactttgaattccattaaaataccttcatagaaagtgttaatgtgtctaccatgccatatgtttatttttactttccccaagttaggtagatagagcaagtcctgctaccataagtttaaaattattaagacaacgggcttttttaaattgctggtagaaccaaaggctaattttgcataggtttatatttttagacttaagaaattagcacctatataataccttcataccttctgcatcactatcaatttttctgtaatttcttatttgaatgcttcctaattctttacagaaaaaattaaaatttgtcacccaaccaggtgataataacaaaacaataatttaggaacatccagtaaatttgaattagttctctaaattttggtaattaatagcttattgataattaaaatcagcctaatgttgcttaagtaggaggaaattgatcagcaatgggattgggtatctattttctttcctataaaagtgctgcctcttctcaaattgctttatttctctcccactgcattagtatatctagaacagcaccttcaactgcaacaaaatataattctcctagatgactaggatatcctccaatctggtatcatgccaaacaacactatgtttggggaacagcacccgaaatgctgctaaaaagctacctcatgtcaagaaaatatggatttcaaaaaatatctggcacagtggcccatgcctataaccccagtactttgggaggccaaggcaggaggattgctggaggccaggagttcaagaccagcctgggcaacatggagagactccgcctctacaaaaaaaaaaaaaacaaatttaatttaattggccagttgtggtggcgtgggcctgtggtcccagctacttgggaagctaaggcaggaggattgctggaggcctggagggcaaggctgcagtgagccatgttcacatcgctgcactccagcctgggtgacagagcaagaccctgtctccaaaaaagcaaaaataaatatctaacaaaaaggaacctgtttccaaaccacagtttcttaactttttcatgatggaatagaaattaaactataagtatatactgataatagctgtgcaggaaattggtttttattttacttaaaagtattttttagaaaaaatgttattctgattgcatagattagcaaaatattgcatcaatataatgttccatttttattaatttttgttatattctaaatgaccgttttataattctgtcttggacaactgggccacagttagaatccacatagacagggaggttcttaggtgtgccatttgtagaataaagggcaatcaagttaatattcggggtaaattttcattgcattctaacctatgaagtttgataaagtatattattattgaacaactctaacactaagatttcctaaaagtaacttacaacaaaaaatgaactatgtgctcaatattcacataaagtcatgattattctcttcttgcattcatataaaagttcccagggggaagaaaatatatctaaaaattagactagaaacttggcactttttatgtattgtagatacactttcatatattgtaaatacatctgttaaagataaaactaagtttctaacatagcactacaaggaaactttggataaatatttttaaaaagaaaatgttaataggataacttaagattttgtatatacttattggaagacaggaagaaactaggcagtaaatattcctacctgacgtgcaaccattgtcaatcaggttttgttaaactgtcaaatttgattttgaaattttagtttgcccaaataatatcttgaaaatgctctgaattttactagaacatattcatagaaagtgtaatgtatctaccaggctctatgtttgtgtttgccaacattagaaaaacaaagtaagttgcgctataatgagtgaaactaaggaaaatgagatttttctgactcccattccctctcttaaaaagtaaaatatttaagctttttttattaaaggaaaaatgggtacagaatactctattgccctccaaatgtccaacatttttaaagccatgttgtccaacaatgtttatacagaaaatagaatgcataggtttaatatagatagcaatgtatatgtaggctcatttgtgggagattgcaaactttatgtgcccccagttctccttggttttgtaggaaagctaaaagaaaaccttgtactctcactaataatgaaattcatagccaatttagaaagggaaaataacctgaaaaccaaaatacctcattcatctgaagatgcctggaataaaatatggaattcttctgtacctacctcccccttacagacttttctcctattttgagagagaaattcaagaagaagaatacagcttgttctctttccacaaatgatactttgtctaagggaataggagataccaagaaacagtatctttaaaaaagtaagctgcccacaggccttcttggagaatgaagaatatcagccccattcagggtttaccaatgtctctctgaggtgacaatacattctggttttccaggacagtcctggcttaggtctgctgtccttctataaatattaacaatgatccctcttatttccaaacgtgtcccagtttagacaacaaattatacagtgtctgcccccaacacacacacacacaccccaccatctttatgggttgttgttgttttcttgttgtttttaattttgtagaattggctttgtcttctccaaagcagataacagcttaaactaaagagcctagctacttctttctgactgagcagggtcatagtaaacagagaagaatggatcacttcctcctctgaaatctccatcaatccactttctgtccaactatctctctacccagcctatttttggtcctagttttcctctcataacccctgggctgtgagtcctgagttctgccttcatatggcagagcaagctagtcattattatgccaggttaggtggtggggcaagacaaaagatagctcttggggacacttgcttggtctgtagagatccacagggcactagtagggtattagagcatttattggtttctttcttccttgagttgagttacttctatcccaaaacagacaggtcttacgttaaccttcaccttaaaaaggatattgccttttttccttctgcttcacaaatttgctccaacactaattcccttgcatttgatttcatcattatgattgttcatcaaccattcttcacgaaaaatcctcacatcacgtcatataaaggttttattatttttaatggaaaataacattgagagattattttcctttttcataaaatttccctttttccctccctctattctcttcactaacattggaaattaaacaagttttacaggctctattgttattaaccctcaaatctattcttcagagaatatatgatcagcctgtgtttactatgcacctactcttataccagaaactgtgaatacaaatacatatatttcctacttctctctttaattggggcacacagtcccatggtccattctgcatttttcaaaattgaatataattgcaacacctgataggttttgccacttagaattctgaaaataaatcaactctgttctaaaccttaagtttccactattattgccaaactctggactgatacaataagccttttaaaaggtgaatttaacacttcacctattatggttatttaagtaaaatgagttttcaaatatttcccccaggagaaatataaaggactatgcaaaaatagaaggagaaaaaggttagttagaggtccagaaggagaattaaaacatggaattgataaattcacctactgatttatgatgttaatgagcaggtacctatacattttatttacttgaaaacattttggccattaactatttctgttttctaaatacatttgtgtttataaaattttaaaatctatatcacaagacttagacatgcctttctttgcaataaagtatatatacctttttacaaatgtaaattttattttgaaacttggcattttctttataattcctaagatatctttgggcacacagactacagataaatctatttaaatcaattcatcattatcactggtttaaatatttaatgtaaccattggaatttttattcttattccttcagagaattctccttttcttctatgtttctgactgcaatacagcccgatattgttattccctatatcttaatgaaaagcaagctgtcagcaaaatgcatttatagtttccagatgtttaacaatctctttcttcttttcttttttgttattatttttatttatgtgaagttagttagctgaggtaccaaaaaagagacaaagacagtattaaaaagtgtgcataaagcacttatttttgtactttagtacaatgttttatagaactttgtgatctatctaaatatattaagtaaaattacaccattcacttgttgggaaaataatctttggtttggaagatattaacataatgggcatcttagaatcataaatcacatgaaatgagagacaatgcaatattgtataattcctggatgatgcaattgttttaattgaattttcaagtgccattataaagttttaaaaattatcaa

SEQ ID NO: 47 - RNA, 9245 nt, Homo sapiens
  source 1..9245: mol_type = mRNA
ataatcaaggaaaccttttccgggtggggatctctgaaattactcagataacagtgctgtgccaaaaacctgtggattttctctacaaaaattattgagcaaccctaattaacctgattttttgctgataatcactctcaatggaatcaaatcacaaatccggggatggattgagcggcactcagaaggaagcagccctccgcgcactggtccagcgcacaggatatagcttggtccaggaaaatggacaaagaaaatatggtggccctccacctggttgggatgctgcaccccctgaaaggggctgtgaaatttttattggaaaacttccccgagacctttttgaggatgagcttataccattatgtgaaaaaatcggtaaaatttatgaaatgagaatgatgatggattttaatggcaacaatagaggatatgcatttgtaacattttcaaataaagtggaagccaagaatgcaatcaagcaacttaataattatgaaattagaaatgggcgcctcttaggggtttgtgccagtgtggacaactgccgattatttgttgggggcatcccaaaaaccaaaaagagagaagaaatcttatcggagatgaaaaaggttactgaaggtgttgtcgatgtcatcgtctacccaagcgctgcagataaaaccaaaaaccgaggctttgccttcgtggagtatgagagtcatcgagcagctgccatggcgaggaggaaactgctaccaggaagaattcagttatggggacatggtattgcagtagactgggcagagccagaagtagaagttgatgaagatacaatgtcttcagtgaaaatcctatatgtaagaaatcttatgctgtctacctctgaagagatgattgaaaaggaattcaacaatatcaaaccaggtgctgtggagagggtgaagaaaattcgagactatgcttttgtgcacttcagtaaccgagaagatgcagttgaggctatgaaagctttaaatggcaaggtgctggatggttcccccattgaagtcaccctagcaaaaccagtggacaaggacagttatgttaggtatacccgaggcacaggtggaaggggcaccatgctgcaaggagagtatacctactctttgggccaagtttatgatcccaccacaacctaccttggagctcctgtcttctatgccccccagacctatgcagcaattcccagtcttcatttcccagccaccaaaggacatctcagcaacagagccattatccgagccccttctgttagagaaatttacatgaatgtacctgtaggggctgcgggagtgagaggactgggcggccgtggctatttggcatacacaggcctgggtcgaggataccaggtcaaaggagacaaaagagaagacaaactctatgacattttacctgggatggagctcaccccaatgaatcctgtcacattaaaaccccaaggaattaaactcgctccccagatattagaagagatttgtcagaaaaataactggggacagccagtgtaccagctgcactctgctattggacaagaccaaagacagctattcttgtacaaaataactattcctgctctagccagccagaatcctgcaatccaccctttcacacctccaaagctgagtgcctttgtggatgaagcaaagacgtatgcagccgaatacaccctgcagaccctgggcatccccactgatggaggcgatggcaccatggctactgctgctgctgctgctactgctttcccaggatatgctgtccctaatgcaactgcacccgtgtctgcagcccagctcaagcaagcggtaacccttggacaagacttagcagcatatacaacctatgaggtctacccaacttttgcagtgactgcccgaggggatggatatggcaccttctgaagatgcttttttaaatttaagaataagacacacaaaactctattaaaaaaaaaaaagaaataaacctctaactcggtccccaatgatcataaataatatgtttcctaaagaaatgcctttccagagactgtatagcttataccaattatagaatcatgaagtaagaacgttattctgaaatgaaatcaaatgttaaagagccttttgtttaacagaaagctggagagccactgccccaaaagttgttcttccagtcttcctgagtgtttggggagtatgtttacaaatcctgccagggtgaagagagatcttgtagctggcaagatttgggagggatggagcagacctgggcagtcagtaataatcaagggaagccaagtaaactccagagtcttacctctggctcacagctgcttttgtgatacacaaagtcacaaagaggaaaagggaagggaggaggttgcttttcaaaatgcattcaccatggctcactgtgtggggcatgattgcaggcctgagctcatgaaagactgtccaagattttgtggtttcaacctctcttcagcctgaaaattccatccagtcgatttgtcacagatcaggatcttcctgtcccctgcagcccatggggacttcctagaaatagatatgatatgttgtagatgtggttaaaacaaatccaggccccattgtttattcagctcattagtccacacctatttttccctagaactcctaccttttccaataatcatgccagcatgaaatgaccttttctactttaaaattttttgacttgttcatattgcttgattttcatcatctcagactcatttttctcctacctaatatcataaaaaagtaaatcagatatacagaatatcatttatgatacttcttcctttcagcaaattgactgaaaagtcattttaaagagagaagtgcagatttgctctcatttctttgatattgttgcagtatccaccccaccatctagcaatcagagatgcttatattaacttgggattagctttgcattatctaacccatttattttaaatctgccaggaaatcctctaactttccttcctttttgtttcagtaagtatcaggcagcttcaccatacctgagtccttttgtcttgaagctgccacagaaaaatcttacagcaatcattgctgattagaaactgtttcagacaatcagcatgggtgttatttaccaaattccccccagagtcctaggcctcttctccagaaatatctgatgatgaagtgaggggagggcaacggtgctacaaaacacggaacagaggtaaagagaaggcactactttcttgccatacttgtaaatgattgctttgttcaaacataaataatcttaagtccaacaccaaatacctgttactcctacatcaatctcattagtggtttaagacacagtactagaattttcattttttaaaatcccttggcccttaaaaaaatttacagaacacaaaggaaaacataaacacaaagacatggaaaattttgtcaactccttaatggaattctgtgatcaaaaagcaggccagattctaatcaaaatcaggtaaattttaatcacaatcagaagtacttgtaacatttcagttgtcctaactccaatgagataacaaagcctccaaggctacagctgaaactctgaaaggccctgtgctttctactttacatttagcgtctaatatttcctaggacagtagttcccaaagtaggctgtacattagaatctcctggagagctttttaaatgctaatgccaataacatatctcataaaatttacactagaatttccttggatgggtgcctggcatcagtattttttaatgatccctaggtgatttcaatgtgcacctaggtttgggaattgctatattcttgtatttatactctatctctttatttcttttcctttttatttcacttttagtaaatagaaaaatgcaattaaaaaatcatctaagttaagacaaatagccaaatcatatttatcccataaatagtatgaatttaaatgtaacttacgtaggcttcaaactaactctaaaaatgagaaagtaaaacaatcttgtcctaaaacaattcttagatccaaatttacactcccctacagatctgcttttattctcaaagacagtgattacattgacataacaaatgtccattggggcaggcaaatttctactgagtctaaaaagcattccaaattctaggagcaaaagttctgattttccttaaaatctttgcaaataaacagcaaagggtattctggatgtcttgtcaatgcaaattaggtttgatttagagaagttttaaatttaatcgcaatgaaaagattagttaatacatgaatctttttatgagaaaaaatcattattctggataaagtcataattgtttgtttacaatattaagagcagacacctaaacatacaataataccaaagtattgagaaaattaccactggaacaccatctcaagttatcacaccatttacctctttagagccagttttgtataagtaggtagaagtggttttggtttttttgctaaaattttactttgttttagtaatatctagggaatactttgaattccattaaaataccttcatagaaagtgttaatgtgtctaccatgccatatgtttatttttactttccccaagttaggtagatagagcaagtcctgctaccataagtttaaaattattaagacaacgggcttttttaaattgctggtagaaccaaaggctaattttgcataggtttatatttttagacttaagaaattagcacctatataataccttcataccttctgcatcactatcaatttttctgtaatttcttatttgaatgcttcctaattctttacagaaaaaattaaaatttgtcacccaaccaggtgataataacaaaacaataatttaggaacatccagtaaatttgaattagttctctaaattttggtaattaatagcttattgataattaaaatcagcctaatgttgcttaagtaggaggaaattgatcagcaatgggattgggtatctattttctttcctataaaagtgctgcctcttctcaaattgctttatttctctcccactgcattagtatatctagaacagcaccttcaactgcaacaaaatataattctcctagatgactaggatatcctccaatctggtatcatgccaaacaacactatgtttggggaacagcacccgaaatgctgctaaaaagctacctcatgtcaagaaaatatggatttcaaaaaatatctggcacagtggcccatgcctataaccccagtactttgggaggccaaggcaggaggattgctggaggccaggagttcaagaccagcctgggcaacatggagagactccgcctctacaaaaaaaaaaaaaacaaatttaatttaattggccagttgtggtggcgtgggcctgtggtcccagctacttgggaagctaaggcaggaggattgctggaggcctggagggcaaggctgcagtgagccatgttcacatcgctgcactccagcctgggtgacagagcaagaccctgtctccaaaaaagcaaaaataaatatctaacaaaaaggaacctgtttccaaaccacagtttcttaactttttcatgatggaatagaaattaaactataagtatatactgataatagctgtgcaggaaattggtttttattttacttaaaagtattttttagaaaaaatgttattctgattgcatagattagcaaaatattgcatcaatataatgttccatttttattaatttttgttatattctaaatgaccgttttataattctgtcttggacaactgggccacagttagaatccacatagacagggaggttcttaggtgtgccatttgtagaataaagggcaatcaagttaatattcggggtaaattttcattgcattctaacctatgaagtttgataaagtatattattattgaacaactctaacactaagatttcctaaaagtaacttacaacaaaaaatgaactatgtgctcaatattcacataaagtcatgattattctcttcttgcattcatataaaagttcccagggggaagaaaatatatctaaaaattagactagaaacttggcactttttatgtattgtagatacactttcatatattgtaaatacatctgttaaagataaaactaagtttctaacatagcactacaaggaaactttggataaatatttttaaaaagaaaatgttaataggataacttaagattttgtatatacttattggaagacaggaagaaactaggcagtaaatattcctacctgacgtgcaaccattgtcaatcaggttttgttaaactgtcaaatttgattttgaaattttagtttgcccaaataatatcttgaaaatgctctgaattttactagaacatattcatagaaagtgtaatgtatctaccaggctctatgtttgtgtttgccaacattagaaaaacaaagtaagttgcgctataatgagtgaaactaaggaaaatgagatttttctgactcccattccctctcttaaaaagtaaaatatttaagctttttttattaaaggaaaaatgggtacagaatactctattgccctccaaatgtccaacatttttaaagccatgttgtccaacaatgtttatacagaaaatagaatgcataggtttaatatagatagcaatgtatatgtaggctcatttgtgggagattgcaaactttatgtgcccccagttctccttggttttgtaggaaagctaaaagaaaaccttgtactctcactaataatgaaattcatagccaatttagaaagggaaaataacctgaaaaccaaaatacctcattcatctgaagatgcctggaataaaatatggaattcttctgtacctacctcccccttacagacttttctcctattttgagagagaaattcaagaagaagaatacagcttgttctctttccacaaatgatactttgtctaagggaataggagataccaagaaacagtatctttaaaaaagtaagctgcccacaggccttcttggagaatgaagaatatcagccccattcagggtttaccaatgtctctctgaggtgacaatacattctggttttccaggacagtcctggcttaggtctgctgtccttctataaatattaacaatgatccctcttatttccaaacgtgtcccagtttagacaacaaattatacagtgtctgcccccaacacacacacacacaccccaccatctttatgggttgttgttgttttcttgttgtttttaattttgtagaattggctttgtcttctccaaagcagataacagcttaaactaaagagcctagctacttctttctgactgagcagggtcatagtaaacagagaagaatggatcacttcctcctctgaaatctccatcaatccactttctgtccaactatctctctacccagcctatttttggtcctagttttcctctcataacccctgggctgtgagtcctgagttctgccttcatatggcagagcaagctagtcattattatgccaggttaggtggtggggcaagacaaaagatagctcttggggacacttgcttggtctgtagagatccacagggcactagtagggtattagagcatttattggtttctttcttccttgagttgagttacttctatcccaaaacagacaggtcttacgttaaccttcaccttaaaaaggatattgccttttttccttctgcttcacaaatttgctccaacactaattcccttgcatttgatttcatcattatgattgttcatcaaccattcttcacgaaaaatcctcacatcacgtcatataaaggttttattatttttaatggaaaataacattgagagattattttcctttttcataaaatttccctttttccctccctctattctcttcactaacattggaaattaaacaagttttacaggctctattgttattaaccctcaaatctattcttcagagaatatatgatcagcctgtgtttactatgcacctactcttataccagaaactgtgaatacaaatacatatatttcctacttctctctttaattggggcacacagtcccatggtccattctgcatttttcaaaattgaatataattgcaacacctgataggttttgccacttagaattctgaaaataaatcaactctgttctaaaccttaagtttccactattattgccaaactctggactgatacaataagccttttaaaaggtgaatttaacacttcacctattatggttatttaagtaaaatgagttttcaaatatttcccccaggagaaatataaaggactatgcaaaaatagaaggagaaaaaggttagttagaggtccagaaggagaattaaaacatggaattgataaattcacctactgatttatgatgttaatgagcaggtacctatacattttatttacttgaaaacattttggccattaactatttctgttttctaaatacatttgtgtttataaaattttaaaatctatatcacaagacttagacatgcctttctttgcaataaagtatatatacctttttacaaatgtaaattttattttgaaacttggcattttctttataattcctaagatatctttgggcacacagactacagataaatctatttaaatcaattcatcattatcactggtttaaatatttaatgtaaccattggaatttttattcttattccttcagagaattctccttttcttctatgtttctgactgcaatacagcccgatattgttattccctatatcttaatgaaaagcaagctgtcagcaaaatgcatttatagtttccagatgtttaacaatctctttcttcttttcttttttgttattatttttatttatgtgaagttagttagctgaggtaccaaaaaagagacaaagacagtattaaaaagtgtgcataaagcacttatttttgtactttagtacaatgttttatagaactttgtgatctatctaaatatattaagtaaaattacaccattcacttgttgggaaaataatctttggtttggaagatattaacataatgggcatcttagaatcataaatcacatgaaatgagagacaatgcaatattgtataattcctggatgatgcaattgttttaattgaattttcaagtgccattataaagttttaaaaattatcaa

SEQ ID NO: 48 - RNA, 9316 nt, Homo sapiens
  source 1..9316: mol_type = mRNA
ataatcaaggaaaccttttccgggtggggatctctgaaattactcagtgagcaaccctaattaacctgattttttgctgataatcactctcaatggaatcaaatcacaaatccggggatggattgagcggcactcagaaggaagcagccctccgcgcactggtccagcgcacaggatatagcttggtccaggaatttagaggttcctatagatggaagcagtgtgtctgggcacatgcccagagccagaagcgagcatgagcacagcaattcctggcctcaaaaaaggaaataatgctttgcagtccatcattttgcaaactttgctggaaaaggaaaatggacaaagaaaatatggtggccctccacctggttgggatgctgcaccccctgaaaggggctgtgaaatttttattggaaaacttccccgagacctttttgaggatgagcttataccattatgtgaaaaaatcggtaaaatttatgaaatgagaatgatgatggattttaatggcaacaatagaggatatgcatttgtaacattttcaaataaagtggaagccaagaatgcaatcaagcaacttaataattatgaaattagaaatgggcgcctcttaggggtttgtgccagtgtggacaactgccgattatttgttgggggcatcccaaaaaccaaaaagagagaagaaatcttatcggagatgaaaaaggttactgaaggtgttgtcgatgtcatcgtctacccaagcgctgcagataaaaccaaaaaccgaggctttgccttcgtggagtatgagagtcatcgagcagctgccatggcgaggaggaaactgctaccaggaagaattcagttatggggacatggtattgcagtagactgggcagagccagaagtagaagttgatgaagatacaatgtcttcagtgaaaatcctatatgtaagaaatcttatgctgtctacctctgaagagatgattgaaaaggaattcaacaatatcaaaccaggtgctgtggagagggtgaagaaaattcgagactatgcttttgtgcacttcagtaaccgagaagatgcagttgaggctatgaaagctttaaatggcaaggtgctggatggttcccccattgaagtcaccctagcaaaaccagtggacaaggacagttatgttaggtatacccgaggcacaggtggaaggggcaccatgctgcaaggagagtatacctactctttgggccaagtttatgatcccaccacaacctaccttggagctcctgtcttctatgccccccagacctatgcagcaattcccagtcttcatttcccagccaccaaaggacatctcagcaacagagccattatccgagccccttctgttagaggggctgcgggagtgagaggactgggcggccgtggctatttggcatacacaggcctgggtcgaggataccaggtcaaaggagacaaaagagaagacaaactctatgacattttacctgggatggagctcaccccaatgaatcctgtcacattaaaaccccaaggaattaaactcgctccccagatattagaagagatttgtcagaaaaataactggggacagccagtgtaccagctgcactctgctattggacaagaccaaagacagctattcttgtacaaaataactattcctgctctagccagccagaatcctgcaatccaccctttcacacctccaaagctgagtgcctttgtggatgaagcaaagacgtatgcagccgaatacaccctgcagaccctgggcatccccactgatggaggcgatggcaccatggctactgctgctgctgctgctactgctttcccaggatatgctgtccctaatgcaactgcacccgtgtctgcagcccagctcaagcaagcggtaacccttggacaagacttagcagcatatacaacctatgaggtctacccaacttttgcagtgactgcccgaggggatggatatggcaccttctgaagatgcttttttaaatttaagaataagacacacaaaactctattaaaaaaaaaaaagaaataaacctctaactcggtccccaatgatcataaataatatgtttcctaaagaaatgcctttccagagactgtatagcttataccaattatagaatcatgaagtaagaacgttattctgaaatgaaatcaaatgttaaagagccttttgtttaacagaaagctggagagccactgccccaaaagttgttcttccagtcttcctgagtgtttggggagtatgtttacaaatcctgccagggtgaagagagatcttgtagctggcaagatttgggagggatggagcagacctgggcagtcagtaataatcaagggaagccaagtaaactccagagtcttacctctggctcacagctgcttttgtgatacacaaagtcacaaagaggaaaagggaagggaggaggttgcttttcaaaatgcattcaccatggctcactgtgtggggcatgattgcaggcctgagctcatgaaagactgtccaagattttgtggtttcaacctctcttcagcctgaaaattccatccagtcgatttgtcacagatcaggatcttcctgtcccctgcagcccatggggacttcctagaaatagatatgatatgttgtagatgtggttaaaacaaatccaggccccattgtttattcagctcattagtccacacctatttttccctagaactcctaccttttccaataatcatgccagcatgaaatgaccttttctactttaaaattttttgacttgttcatattgcttgattttcatcatctcagactcatttttctcctacctaatatcataaaaaagtaaatcagatatacagaatatcatttatgatacttcttcctttcagcaaattgactgaaaagtcattttaaagagagaagtgcagatttgctctcatttctttgatattgttgcagtatccaccccaccatctagcaatcagagatgcttatattaacttgggattagctttgcattatctaacccatttattttaaatctgccaggaaatcctctaactttccttcctttttgtttcagtaagtatcaggcagcttcaccatacctgagtccttttgtcttgaagctgccacagaaaaatcttacagcaatcattgctgattagaaactgtttcagacaatcagcatgggtgttatttaccaaattccccccagagtcctaggcctcttctccagaaatatctgatgatgaagtgaggggagggcaacggtgctacaaaacacggaacagaggtaaagagaaggcactactttcttgccatacttgtaaatgattgctttgttcaaacataaataatcttaagtccaacaccaaatacctgttactcctacatcaatctcattagtggtttaagacacagtactagaattttcattttttaaaatcccttggcccttaaaaaaatttacagaacacaaaggaaaacataaacacaaagacatggaaaattttgtcaactccttaatggaattctgtgatcaaaaagcaggccagattctaatcaaaatcaggtaaattttaatcacaatcagaagtacttgtaacatttcagttgtcctaactccaatgagataacaaagcctccaaggctacagctgaaactctgaaaggccctgtgctttctactttacatttagcgtctaatatttcctaggacagtagttcccaaagtaggctgtacattagaatctcctggagagctttttaaatgctaatgccaataacatatctcataaaatttacactagaatttccttggatgggtgcctggcatcagtattttttaatgatccctaggtgatttcaatgtgcacctaggtttgggaattgctatattcttgtatttatactctatctctttatttcttttcctttttatttcacttttagtaaatagaaaaatgcaattaaaaaatcatctaagttaagacaaatagccaaatcatatttatcccataaatagtatgaatttaaatgtaacttacgtaggcttcaaactaactctaaaaatgagaaagtaaaacaatcttgtcctaaaacaattcttagatccaaatttacactcccctacagatctgcttttattctcaaagacagtgattacattgacataacaaatgtccattggggcaggcaaatttctactgagtctaaaaagcattccaaattctaggagcaaaagttctgattttccttaaaatctttgcaaataaacagcaaagggtattctggatgtcttgtcaatgcaaattaggtttgatttagagaagttttaaatttaatcgcaatgaaaagattagttaatacatgaatctttttatgagaaaaaatcattattctggataaagtcataattgtttgtttacaatattaagagcagacacctaaacatacaataataccaaagtattgagaaaattaccactggaacaccatctcaagttatcacaccatttacctctttagagccagttttgtataagtaggtagaagtggttttggtttttttgctaaaattttactttgttttagtaatatctagggaatactttgaattccattaaaataccttcatagaaagtgttaatgtgtctaccatgccatatgtttatttttactttccccaagttaggtagatagagcaagtcctgctaccataagtttaaaattattaagacaacgggcttttttaaattgctggtagaaccaaaggctaattttgcataggtttatatttttagacttaagaaattagcacctatataataccttcataccttctgcatcactatcaatttttctgtaatttcttatttgaatgcttcctaattctttacagaaaaaattaaaatttgtcacccaaccaggtgataataacaaaacaataatttaggaacatccagtaaatttgaattagttctctaaattttggtaattaatagcttattgataattaaaatcagcctaatgttgcttaagtaggaggaaattgatcagcaatgggattgggtatctattttctttcctataaaagtgctgcctcttctcaaattgctttatttctctcccactgcattagtatatctagaacagcaccttcaactgcaacaaaatataattctcctagatgactaggatatcctccaatctggtatcatgccaaacaacactatgtttggggaacagcacccgaaatgctgctaaaaagctacctcatgtcaagaaaatatggatttcaaaaaatatctggcacagtggcccatgcctataaccccagtactttgggaggccaaggcaggaggattgctggaggccaggagttcaagaccagcctgggcaacatggagagactccgcctctacaaaaaaaaaaaaaacaaatttaatttaattggccagttgtggtggcgtgggcctgtggtcccagctacttgggaagctaaggcaggaggattgctggaggcctggagggcaaggctgcagtgagccatgttcacatcgctgcactccagcctgggtgacagagcaagaccctgtctccaaaaaagcaaaaataaatatctaacaaaaaggaacctgtttccaaaccacagtttcttaactttttcatgatggaatagaaattaaactataagtatatactgataatagctgtgcaggaaattggtttttattttacttaaaagtattttttagaaaaaatgttattctgattgcatagattagcaaaatattgcatcaatataatgttccatttttattaatttttgttatattctaaatgaccgttttataattctgtcttggacaactgggccacagttagaatccacatagacagggaggttcttaggtgtgccatttgtagaataaagggcaatcaagttaatattcggggtaaattttcattgcattctaacctatgaagtttgataaagtatattattattgaacaactctaacactaagatttcctaaaagtaacttacaacaaaaaatgaactatgtgctcaatattcacataaagtcatgattattctcttcttgcattcatataaaagttcccagggggaagaaaatatatctaaaaattagactagaaacttggcactttttatgtattgtagatacactttcatatattgtaaatacatctgttaaagataaaactaagtttctaacatagcactacaaggaaactttggataaatatttttaaaaagaaaatgttaataggataacttaagattttgtatatacttattggaagacaggaagaaactaggcagtaaatattcctacctgacgtgcaaccattgtcaatcaggttttgttaaactgtcaaatttgattttgaaattttagtttgcccaaataatatcttgaaaatgctctgaattttactagaacatattcatagaaagtgtaatgtatctaccaggctctatgtttgtgtttgccaacattagaaaaacaaagtaagttgcgctataatgagtgaaactaaggaaaatgagatttttctgactcccattccctctcttaaaaagtaaaatatttaagctttttttattaaaggaaaaatgggtacagaatactctattgccctccaaatgtccaacatttttaaagccatgttgtccaacaatgtttatacagaaaatagaatgcataggtttaatatagatagcaatgtatatgtaggctcatttgtgggagattgcaaactttatgtgcccccagttctccttggttttgtaggaaagctaaaagaaaaccttgtactctcactaataatgaaattcatagccaatttagaaagggaaaataacctgaaaaccaaaatacctcattcatctgaagatgcctggaataaaatatggaattcttctgtacctacctcccccttacagacttttctcctattttgagagagaaattcaagaagaagaatacagcttgttctctttccacaaatgatactttgtctaagggaataggagataccaagaaacagtatctttaaaaaagtaagctgcccacaggccttcttggagaatgaagaatatcagccccattcagggtttaccaatgtctctctgaggtgacaatacattctggttttccaggacagtcctggcttaggtctgctgtccttctataaatattaacaatgatccctcttatttccaaacgtgtcccagtttagacaacaaattatacagtgtctgcccccaacacacacacacacaccccaccatctttatgggttgttgttgttttcttgttgtttttaattttgtagaattggctttgtcttctccaaagcagataacagcttaaactaaagagcctagctacttctttctgactgagcagggtcatagtaaacagagaagaatggatcacttcctcctctgaaatctccatcaatccactttctgtccaactatctctctacccagcctatttttggtcctagttttcctctcataacccctgggctgtgagtcctgagttctgccttcatatggcagagcaagctagtcattattatgccaggttaggtggtggggcaagacaaaagatagctcttggggacacttgcttggtctgtagagatccacagggcactagtagggtattagagcatttattggtttctttcttccttgagttgagttacttctatcccaaaacagacaggtcttacgttaaccttcaccttaaaaaggatattgccttttttccttctgcttcacaaatttgctccaacactaattcccttgcatttgatttcatcattatgattgttcatcaaccattcttcacgaaaaatcctcacatcacgtcatataaaggttttattatttttaatggaaaataacattgagagattattttcctttttcataaaatttccctttttccctccctctattctcttcactaacattggaaattaaacaagttttacaggctctattgttattaaccctcaaatctattcttcagagaatatatgatcagcctgtgtttactatgcacctactcttataccagaaactgtgaatacaaatacatatatttcctacttctctctttaattggggcacacagtcccatggtccattctgcatttttcaaaattgaatataattgcaacacctgataggttttgccacttagaattctgaaaataaatcaactctgttctaaaccttaagtttccactattattgccaaactctggactgatacaataagccttttaaaaggtgaatttaacacttcacctattatggttatttaagtaaaatgagttttcaaatatttcccccaggagaaatataaaggactatgcaaaaatagaaggagaaaaaggttagttagaggtccagaaggagaattaaaacatggaattgataaattcacctactgatttatgatgttaatgagcaggtacctatacattttatttacttgaaaacattttggccattaactatttctgttttctaaatacatttgtgtttataaaattttaaaatctatatcacaagacttagacatgcctttctttgcaataaagtatatatacctttttacaaatgtaaattttattttgaaacttggcattttctttataattcctaagatatctttgggcacacagactacagataaatctatttaaatcaattcatcattatcactggtttaaatatttaatgtaaccattggaatttttattcttattccttcagagaattctccttttcttctatgtttctgactgcaatacagcccgatattgttattccctatatcttaatgaaaagcaagctgtcagcaaaatgcatttatagtttccagatgtttaacaatctctttcttcttttcttttttgttattatttttatttatgtgaagttagttagctgaggtaccaaaaaagagacaaagacagtattaaaaagtgtgcataaagcacttatttttgtactttagtacaatgttttatagaactttgtgatctatctaaatatattaagtaaaattacaccattcacttgttgggaaaataatctttggtttggaagatattaacataatgggcatcttagaatcataaatcacatgaaatgagagacaatgcaatattgtataattcctggatgatgcaattgttttaattgaattttcaagtgccattataaagttttaaaaattatcaa

SEQ ID NO: 49 - RNA, 9314 nt, Homo sapiens
  source 1..9314: mol_type = mRNA
ataatcaaggaaaccttttccgggtggggatctctgaaattactcagataacagtgctgtgccaaaaacctgtggattttctctacaaaaattatcatgactgttatctttttgatggaaaagatgaagttcagcgaagaacaaatctacaacttggccagaagactgtagactttgccttcaaagagtgagcaaccctaattaacctgattttttgctgataatcactctcaatggaatcaaatcacaaatccggggatggattgagcggcactcagaaggaagcagccctccgcgcactggtccagcgcacaggatatagcttggtccaggaaaatggacaaagaaaatatggtggccctccacctggttgggatgctgcaccccctgaaaggggctgtgaaatttttattggaaaacttccccgagacctttttgaggatgagcttataccattatgtgaaaaaatcggtaaaatttatgaaatgagaatgatgatggattttaatggcaacaatagaggatatgcatttgtaacattttcaaataaagtggaagccaagaatgcaatcaagcaacttaataattatgaaattagaaatgggcgcctcttaggggtttgtgccagtgtggacaactgccgattatttgttgggggcatcccaaaaaccaaaaagagagaagaaatcttatcggagatgaaaaaggttactgaaggtgttgtcgatgtcatcgtctacccaagcgctgcagataaaaccaaaaaccgaggctttgccttcgtggagtatgagagtcatcgagcagctgccatggcgaggaggaaactgctaccaggaagaattcagttatggggacatggtattgcagtagactgggcagagccagaagtagaagttgatgaagatacaatgtcttcagtgaaaatcctatatgtaagaaatcttatgctgtctacctctgaagagatgattgaaaaggaattcaacaatatcaaaccaggtgctgtggagagggtgaagaaaattcgagactatgcttttgtgcacttcagtaaccgagaagatgcagttgaggctatgaaagctttaaatggcaaggtgctggatggttcccccattgaagtcaccctagcaaaaccagtggacaaggacagttatgttaggtatacccgaggcacaggtggaaggggcaccatgctgcaaggagagtatacctactctttgggccaagtttatgatcccaccacaacctaccttggagctcctgtcttctatgccccccagacctatgcagcaattcccagtcttcatttcccagccaccaaaggacatctcagcaacagagccattatccgagccccttctgttagaggggctgcgggagtgagaggactgggcggccgtggctatttggcatacacaggcctgggtcgaggataccaggtcaaaggagacaaaagagaagacaaactctatgacattttacctgggatggagctcaccccaatgaatcctgtcacattaaaaccccaaggaattaaactcgctccccagatattagaagagatttgtcagaaaaataactggggacagccagtgtaccagctgcactctgctattggacaagaccaaagacagctattcttgtacaaaataactattcctgctctagccagccagaatcctgcaatccaccctttcacacctccaaagctgagtgcctttgtggatgaagcaaagacgtatgcagccgaatacaccctgcagaccctgggcatccccactgatggaggcgatggcaccatggctactgctgctgctgctgctactgctttcccaggatatgctgtccctaatgcaactgcacccgtgtctgcagcccagctcaagcaagcggtaacccttggacaagacttagcagcatatacaacctatgaggtctacccaacttttgcagtgactgcccgaggggatggatatggcaccttctgaagatgcttttttaaatttaagaataagacacacaaaactctattaaaaaaaaaaaagaaataaacctctaactcggtccccaatgatcataaataatatgtttcctaaagaaatgcctttccagagactgtatagcttataccaattatagaatcatgaagtaagaacgttattctgaaatgaaatcaaatgttaaagagccttttgtttaacagaaagctggagagccactgccccaaaagttgttcttccagtcttcctgagtgtttggggagtatgtttacaaatcctgccagggtgaagagagatcttgtagctggcaagatttgggagggatggagcagacctgggcagtcagtaataatcaagggaagccaagtaaactccagagtcttacctctggctcacagctgcttttgtgatacacaaagtcacaaagaggaaaagggaagggaggaggttgcttttcaaaatgcattcaccatggctcactgtgtggggcatgattgcaggcctgagctcatgaaagactgtccaagattttgtggtttcaacctctcttcagcctgaaaattccatccagtcgatttgtcacagatcaggatcttcctgtcccctgcagcccatggggacttcctagaaatagatatgatatgttgtagatgtggttaaaacaaatccaggccccattgtttattcagctcattagtccacacctatttttccctagaactcctaccttttccaataatcatgccagcatgaaatgaccttttctactttaaaattttttgacttgttcatattgcttgattttcatcatctcagactcatttttctcctacctaatatcataaaaaagtaaatcagatatacagaatatcatttatgatacttcttcctttcagcaaattgactgaaaagtcattttaaagagagaagtgcagatttgctctcatttctttgatattgttgcagtatccaccccaccatctagcaatcagagatgcttatattaacttgggattagctttgcattatctaacccatttattttaaatctgccaggaaatcctctaactttccttcctttttgtttcagtaagtatcaggcagcttcaccatacctgagtccttttgtcttgaagctgccacagaaaaatcttacagcaatcattgctgattagaaactgtttcagacaatcagcatgggtgttatttaccaaattccccccagagtcctaggcctcttctccagaaatatctgatgatgaagtgaggggagggcaacggtgctacaaaacacggaacagaggtaaagagaaggcactactttcttgccatacttgtaaatgattgctttgttcaaacataaataatcttaagtccaacaccaaatacctgttactcctacatcaatctcattagtggtttaagacacagtactagaattttcattttttaaaatcccttggcccttaaaaaaatttacagaacacaaaggaaaacataaacacaaagacatggaaaattttgtcaactccttaatggaattctgtgatcaaaaagcaggccagattctaatcaaaatcaggtaaattttaatcacaatcagaagtacttgtaacatttcagttgtcctaactccaatgagataacaaagcctccaaggctacagctgaaactctgaaaggccctgtgctttctactttacatttagcgtctaatatttcctaggacagtagttcccaaagtaggctgtacattagaatctcctggagagctttttaaatgctaatgccaataacatatctcataaaatttacactagaatttccttggatgggtgcctggcatcagtattttttaatgatccctaggtgatttcaatgtgcacctaggtttgggaattgctatattcttgtatttatactctatctctttatttcttttcctttttatttcacttttagtaaatagaaaaatgcaattaaaaaatcatctaagttaagacaaatagccaaatcatatttatcccataaatagtatgaatttaaatgtaacttacgtaggcttcaaactaactctaaaaatgagaaagtaaaacaatcttgtcctaaaacaattcttagatccaaatttacactcccctacagatctgcttttattctcaaagacagtgattacattgacataacaaatgtccattggggcaggcaaatttctactgagtctaaaaagcattccaaattctaggagcaaaagttctgattttccttaaaatctttgcaaataaacagcaaagggtattctggatgtcttgtcaatgcaaattaggtttgatttagagaagttttaaatttaatcgcaatgaaaagattagttaatacatgaatctttttatgagaaaaaatcattattctggataaagtcataattgtttgtttacaatattaagagcagacacctaaacatacaataataccaaagtattgagaaaattaccactggaacaccatctcaagttatcacaccatttacctctttagagccagttttgtataagtaggtagaagtggttttggtttttttgctaaaattttactttgttttagtaatatctagggaatactttgaattccattaaaataccttcatagaaagtgttaatgtgtctaccatgccatatgtttatttttactttccccaagttaggtagatagagcaagtcctgctaccataagtttaaaattattaagacaacgggcttttttaaattgctggtagaaccaaaggctaattttgcataggtttatatttttagacttaagaaattagcacctatataataccttcataccttctgcatcactatcaatttttctgtaatttcttatttgaatgcttcctaattctttacagaaaaaattaaaatttgtcacccaaccaggtgataataacaaaacaataatttaggaacatccagtaaatttgaattagttctctaaattttggtaattaatagcttattgataattaaaatcagcctaatgttgcttaagtaggaggaaattgatcagcaatgggattgggtatctattttctttcctataaaagtgctgcctcttctcaaattgctttatttctctcccactgcattagtatatctagaacagcaccttcaactgcaacaaaatataattctcctagatgactaggatatcctccaatctggtatcatgccaaacaacactatgtttggggaacagcacccgaaatgctgctaaaaagctacctcatgtcaagaaaatatggatttcaaaaaatatctggcacagtggcccatgcctataaccccagtactttgggaggccaaggcaggaggattgctggaggccaggagttcaagaccagcctgggcaacatggagagactccgcctctacaaaaaaaaaaaaaacaaatttaatttaattggccagttgtggtggcgtgggcctgtggtcccagctacttgggaagctaaggcaggaggattgctggaggcctggagggcaaggctgcagtgagccatgttcacatcgctgcactccagcctgggtgacagagcaagaccctgtctccaaaaaagcaaaaataaatatctaacaaaaaggaacctgtttccaaaccacagtttcttaactttttcatgatggaatagaaattaaactataagtatatactgataatagctgtgcaggaaattggtttttattttacttaaaagtattttttagaaaaaatgttattctgattgcatagattagcaaaatattgcatcaatataatgttccatttttattaatttttgttatattctaaatgaccgttttataattctgtcttggacaactgggccacagttagaatccacatagacagggaggttcttaggtgtgccatttgtagaataaagggcaatcaagttaatattcggggtaaattttcattgcattctaacctatgaagtttgataaagtatattattattgaacaactctaacactaagatttcctaaaagtaacttacaacaaaaaatgaactatgtgctcaatattcacataaagtcatgattattctcttcttgcattcatataaaagttcccagggggaagaaaatatatctaaaaattagactagaaacttggcactttttatgtattgtagatacactttcatatattgtaaatacatctgttaaagataaaactaagtttctaacatagcactacaaggaaactttggataaatatttttaaaaagaaaatgttaataggataacttaagattttgtatatacttattggaagacaggaagaaactaggcagtaaatattcctacctgacgtgcaaccattgtcaatcaggttttgttaaactgtcaaatttgattttgaaattttagtttgcccaaataatatcttgaaaatgctctgaattttactagaacatattcatagaaagtgtaatgtatctaccaggctctatgtttgtgtttgccaacattagaaaaacaaagtaagttgcgctataatgagtgaaactaaggaaaatgagatttttctgactcccattccctctcttaaaaagtaaaatatttaagctttttttattaaaggaaaaatgggtacagaatactctattgccctccaaatgtccaacatttttaaagccatgttgtccaacaatgtttatacagaaaatagaatgcataggtttaatatagatagcaatgtatatgtaggctcatttgtgggagattgcaaactttatgtgcccccagttctccttggttttgtaggaaagctaaaagaaaaccttgtactctcactaataatgaaattcatagccaatttagaaagggaaaataacctgaaaaccaaaatacctcattcatctgaagatgcctggaataaaatatggaattcttctgtacctacctcccccttacagacttttctcctattttgagagagaaattcaagaagaagaatacagcttgttctctttccacaaatgatactttgtctaagggaataggagataccaagaaacagtatctttaaaaaagtaagctgcccacaggccttcttggagaatgaagaatatcagccccattcagggtttaccaatgtctctctgaggtgacaatacattctggttttccaggacagtcctggcttaggtctgctgtccttctataaatattaacaatgatccctcttatttccaaacgtgtcccagtttagacaacaaattatacagtgtctgcccccaacacacacacacacaccccaccatctttatgggttgttgttgttttcttgttgtttttaattttgtagaattggctttgtcttctccaaagcagataacagcttaaactaaagagcctagctacttctttctgactgagcagggtcatagtaaacagagaagaatggatcacttcctcctctgaaatctccatcaatccactttctgtccaactatctctctacccagcctatttttggtcctagttttcctctcataacccctgggctgtgagtcctgagttctgccttcatatggcagagcaagctagtcattattatgccaggttaggtggtggggcaagacaaaagatagctcttggggacacttgcttggtctgtagagatccacagggcactagtagggtattagagcatttattggtttctttcttccttgagttgagttacttctatcccaaaacagacaggtcttacgttaaccttcaccttaaaaaggatattgccttttttccttctgcttcacaaatttgctccaacactaattcccttgcatttgatttcatcattatgattgttcatcaaccattcttcacgaaaaatcctcacatcacgtcatataaaggttttattatttttaatggaaaataacattgagagattattttcctttttcataaaatttccctttttccctccctctattctcttcactaacattggaaattaaacaagttttacaggctctattgttattaaccctcaaatctattcttcagagaatatatgatcagcctgtgtttactatgcacctactcttataccagaaactgtgaatacaaatacatatatttcctacttctctctttaattggggcacacagtcccatggtccattctgcatttttcaaaattgaatataattgcaacacctgataggttttgccacttagaattctgaaaataaatcaactctgttctaaaccttaagtttccactattattgccaaactctggactgatacaataagccttttaaaaggtgaatttaacacttcacctattatggttatttaagtaaaatgagttttcaaatatttcccccaggagaaatataaaggactatgcaaaaatagaaggagaaaaaggttagttagaggtccagaaggagaattaaaacatggaattgataaattcacctactgatttatgatgttaatgagcaggtacctatacattttatttacttgaaaacattttggccattaactatttctgttttctaaatacatttgtgtttataaaattttaaaatctatatcacaagacttagacatgcctttctttgcaataaagtatatatacctttttacaaatgtaaattttattttgaaacttggcattttctttataattcctaagatatctttgggcacacagactacagataaatctatttaaatcaattcatcattatcactggtttaaatatttaatgtaaccattggaatttttattcttattccttcagagaattctccttttcttctatgtttctgactgcaatacagcccgatattgttattccctatatcttaatgaaaagcaagctgtcagcaaaatgcatttatagtttccagatgtttaacaatctctttcttcttttcttttttgttattatttttatttatgtgaagttagttagctgaggtaccaaaaaagagacaaagacagtattaaaaagtgtgcataaagcacttatttttgtactttagtacaatgttttatagaactttgtgatctatctaaatatattaagtaaaattacaccattcacttgttgggaaaataatctttggtttggaagatattaacataatgggcatcttagaatcataaatcacatgaaatgagagacaatgcaatattgtataattcctggatgatgcaattgttttaattgaattttcaagtgccattataaagttttaaaaattatcaa

SEQ ID NO: 50 - RNA, 9481 nt, Homo sapiens
  source 1..9481: mol_type = mRNA
ataatcaaggaaaccttttccgggtggggatctctgaaattactcagataacagtgctgtgccaaaaacctgtggattttctctacaaaaattatcatgactgttatctttttgatggaaaagatgaagttcagcgaagaacaaatctacaacttggccagaagactgtagactttgccttcaaagagtgagcaaccctaattaacctgattttttgctgataatcactctcaatggaatcaaatcacaaatccggggatggattgagcggcactcagaaggaagcagccctccgcgcactggtccagcgcacaggatatagcttggtccaggaatttagaggttcctatagatggaagcagtgtgtctgggcacatgcccagagccagaagcgagcatgagcacagcaattcctggcctcaaaaaaggaaataatgctttgcagtccatcattttgcaaactttgctggaaaaggaaaatggacaaagaaaatatggtggccctccacctggttgggatgctgcaccccctgaaaggggctgtgaaatttttattggaaaacttccccgagacctttttgaggatgagcttataccattatgtgaaaaaatcggtaaaatttatgaaatgagaatgatgatggattttaatggcaacaatagaggatatgcatttgtaacattttcaaataaagtggaagccaagaatgcaatcaagcaacttaataattatgaaattagaaatgggcgcctcttaggggtttgtgccagtgtggacaactgccgattatttgttgggggcatcccaaaaaccaaaaagagagaagaaatcttatcggagatgaaaaaggttactgaaggtgttgtcgatgtcatcgtctacccaagcgctgcagataaaaccaaaaaccgaggctttgccttcgtggagtatgagagtcatcgagcagctgccatggcgaggaggaaactgctaccaggaagaattcagttatggggacatggtattgcagtagactgggcagagccagaagtagaagttgatgaagatacaatgtcttcagtgaaaatcctatatgtaagaaatcttatgctgtctacctctgaagagatgattgaaaaggaattcaacaatatcaaaccaggtgctgtggagagggtgaagaaaattcgagactatgcttttgtgcacttcagtaaccgagaagatgcagttgaggctatgaaagctttaaatggcaaggtgctggatggttcccccattgaagtcaccctagcaaaaccagtggacaaggacagttatgttaggtatacccgaggcacaggtggaaggggcaccatgctgcaaggagagtatacctactctttgggccaagtttatgatcccaccacaacctaccttggagctcctgtcttctatgccccccagacctatgcagcaattcccagtcttcatttcccagccaccaaaggacatctcagcaacagagccattatccgagccccttctgttagagaaatttacatgaatgtacctgtaggggctgcgggagtgagaggactgggcggccgtggctatttggcatacacaggcctgggtcgaggataccaggtcaaaggagacaaaagagaagacaaactctatgacattttacctgggatggagctcaccccaatgaatcctgtcacattaaaaccccaaggaattaaactcgctccccagatattagaagagatttgtcagaaaaataactggggacagccagtgtaccagctgcactctgctattggacaagaccaaagacagctattcttgtacaaaataactattcctgctctagccagccagaatcctgcaatccaccctttcacacctccaaagctgagtgcctttgtggatgaagcaaagacgtatgcagccgaatacaccctgcagaccctgggcatccccactgatggaggcgatggcaccatggctactgctgctgctgctgctactgctttcccaggatatgctgtccctaatgcaactgcacccgtgtctgcagcccagctcaagcaagcggtaacccttggacaagacttagcagcatatacaacctatgaggtctacccaacttttgcagtgactgcccgaggggatggatatggcaccttctgaagatgcttttttaaatttaagaataagacacacaaaactctattaaaaaaaaaaaagaaataaacctctaactcggtccccaatgatcataaataatatgtttcctaaagaaatgcctttccagagactgtatagcttataccaattatagaatcatgaagtaagaacgttattctgaaatgaaatcaaatgttaaagagccttttgtttaacagaaagctggagagccactgccccaaaagttgttcttccagtcttcctgagtgtttggggagtatgtttacaaatcctgccagggtgaagagagatcttgtagctggcaagatttgggagggatggagcagacctgggcagtcagtaataatcaagggaagccaagtaaactccagagtcttacctctggctcacagctgcttttgtgatacacaaagtcacaaagaggaaaagggaagggaggaggttgcttttcaaaatgcattcaccatggctcactgtgtggggcatgattgcaggcctgagctcatgaaagactgtccaagattttgtggtttcaacctctcttcagcctgaaaattccatccagtcgatttgtcacagatcaggatcttcctgtcccctgcagcccatggggacttcctagaaatagatatgatatgttgtagatgtggttaaaacaaatccaggccccattgtttattcagctcattagtccacacctatttttccctagaactcctaccttttccaataatcatgccagcatgaaatgaccttttctactttaaaattttttgacttgttcatattgcttgattttcatcatctcagactcatttttctcctacctaatatcataaaaaagtaaatcagatatacagaatatcatttatgatacttcttcctttcagcaaattgactgaaaagtcattttaaagagagaagtgcagatttgctctcatttctttgatattgttgcagtatccaccccaccatctagcaatcagagatgcttatattaacttgggattagctttgcattatctaacccatttattttaaatctgccaggaaatcctctaactttccttcctttttgtttcagtaagtatcaggcagcttcaccatacctgagtccttttgtcttgaagctgccacagaaaaatcttacagcaatcattgctgattagaaactgtttcagacaatcagcatgggtgttatttaccaaattccccccagagtcctaggcctcttctccagaaatatctgatgatgaagtgaggggagggcaacggtgctacaaaacacggaacagaggtaaagagaaggcactactttcttgccatacttgtaaatgattgctttgttcaaacataaataatcttaagtccaacaccaaatacctgttactcctacatcaatctcattagtggtttaagacacagtactagaattttcattttttaaaatcccttggcccttaaaaaaatttacagaacacaaaggaaaacataaacacaaagacatggaaaattttgtcaactccttaatggaattctgtgatcaaaaagcaggccagattctaatcaaaatcaggtaaattttaatcacaatcagaagtacttgtaacatttcagttgtcctaactccaatgagataacaaagcctccaaggctacagctgaaactctgaaaggccctgtgctttctactttacatttagcgtctaatatttcctaggacagtagttcccaaagtaggctgtacattagaatctcctggagagctttttaaatgctaatgccaataacatatctcataaaatttacactagaatttccttggatgggtgcctggcatcagtattttttaatgatccctaggtgatttcaatgtgcacctaggtttgggaattgctatattcttgtatttatactctatctctttatttcttttcctttttatttcacttttagtaaatagaaaaatgcaattaaaaaatcatctaagttaagacaaatagccaaatcatatttatcccataaatagtatgaatttaaatgtaacttacgtaggcttcaaactaactctaaaaatgagaaagtaaaacaatcttgtcctaaaacaattcttagatccaaatttacactcccctacagatctgcttttattctcaaagacagtgattacattgacataacaaatgtccattggggcaggcaaatttctactgagtctaaaaagcattccaaattctaggagcaaaagttctgattttccttaaaatctttgcaaataaacagcaaagggtattctggatgtcttgtcaatgcaaattaggtttgatttagagaagttttaaatttaatcgcaatgaaaagattagttaatacatgaatctttttatgagaaaaaatcattattctggataaagtcataattgtttgtttacaatattaagagcagacacctaaacatacaataataccaaagtattgagaaaattaccactggaacaccatctcaagttatcacaccatttacctctttagagccagttttgtataagtaggtagaagtggttttggtttttttgctaaaattttactttgttttagtaatatctagggaatactttgaattccattaaaataccttcatagaaagtgttaatgtgtctaccatgccatatgtttatttttactttccccaagttaggtagatagagcaagtcctgctaccataagtttaaaattattaagacaacgggcttttttaaattgctggtagaaccaaaggctaattttgcataggtttatatttttagacttaagaaattagcacctatataataccttcataccttctgcatcactatcaatttttctgtaatttcttatttgaatgcttcctaattctttacagaaaaaattaaaatttgtcacccaaccaggtgataataacaaaacaataatttaggaacatccagtaaatttgaattagttctctaaattttggtaattaatagcttattgataattaaaatcagcctaatgttgcttaagtaggaggaaattgatcagcaatgggattgggtatctattttctttcctataaaagtgctgcctcttctcaaattgctttatttctctcccactgcattagtatatctagaacagcaccttcaactgcaacaaaatataattctcctagatgactaggatatcctccaatctggtatcatgccaaacaacactatgtttggggaacagcacccgaaatgctgctaaaaagctacctcatgtcaagaaaatatggatttcaaaaaatatctggcacagtggcccatgcctataaccccagtactttgggaggccaaggcaggaggattgctggaggccaggagttcaagaccagcctgggcaacatggagagactccgcctctacaaaaaaaaaaaaaacaaatttaatttaattggccagttgtggtggcgtgggcctgtggtcccagctacttgggaagctaaggcaggaggattgctggaggcctggagggcaaggctgcagtgagccatgttcacatcgctgcactccagcctgggtgacagagcaagaccctgtctccaaaaaagcaaaaataaatatctaacaaaaaggaacctgtttccaaaccacagtttcttaactttttcatgatggaatagaaattaaactataagtatatactgataatagctgtgcaggaaattggtttttattttacttaaaagtattttttagaaaaaatgttattctgattgcatagattagcaaaatattgcatcaatataatgttccatttttattaatttttgttatattctaaatgaccgttttataattctgtcttggacaactgggccacagttagaatccacatagacagggaggttcttaggtgtgccatttgtagaataaagggcaatcaagttaatattcggggtaaattttcattgcattctaacctatgaagtttgataaagtatattattattgaacaactctaacactaagatttcctaaaagtaacttacaacaaaaaatgaactatgtgctcaatattcacataaagtcatgattattctcttcttgcattcatataaaagttcccagggggaagaaaatatatctaaaaattagactagaaacttggcactttttatgtattgtagatacactttcatatattgtaaatacatctgttaaagataaaactaagtttctaacatagcactacaaggaaactttggataaatatttttaaaaagaaaatgttaataggataacttaagattttgtatatacttattggaagacaggaagaaactaggcagtaaatattcctacctgacgtgcaaccattgtcaatcaggttttgttaaactgtcaaatttgattttgaaattttagtttgcccaaataatatcttgaaaatgctctgaattttactagaacatattcatagaaagtgtaatgtatctaccaggctctatgtttgtgtttgccaacattagaaaaacaaagtaagttgcgctataatgagtgaaactaaggaaaatgagatttttctgactcccattccctctcttaaaaagtaaaatatttaagctttttttattaaaggaaaaatgggtacagaatactctattgccctccaaatgtccaacatttttaaagccatgttgtccaacaatgtttatacagaaaatagaatgcataggtttaatatagatagcaatgtatatgtaggctcatttgtgggagattgcaaactttatgtgcccccagttctccttggttttgtaggaaagctaaaagaaaaccttgtactctcactaataatgaaattcatagccaatttagaaagggaaaataacctgaaaaccaaaatacctcattcatctgaagatgcctggaataaaatatggaattcttctgtacctacctcccccttacagacttttctcctattttgagagagaaattcaagaagaagaatacagcttgttctctttccacaaatgatactttgtctaagggaataggagataccaagaaacagtatctttaaaaaagtaagctgcccacaggccttcttggagaatgaagaatatcagccccattcagggtttaccaatgtctctctgaggtgacaatacattctggttttccaggacagtcctggcttaggtctgctgtccttctataaatattaacaatgatccctcttatttccaaacgtgtcccagtttagacaacaaattatacagtgtctgcccccaacacacacacacacaccccaccatctttatgggttgttgttgttttcttgttgtttttaattttgtagaattggctttgtcttctccaaagcagataacagcttaaactaaagagcctagctacttctttctgactgagcagggtcatagtaaacagagaagaatggatcacttcctcctctgaaatctccatcaatccactttctgtccaactatctctctacccagcctatttttggtcctagttttcctctcataacccctgggctgtgagtcctgagttctgccttcatatggcagagcaagctagtcattattatgccaggttaggtggtggggcaagacaaaagatagctcttggggacacttgcttggtctgtagagatccacagggcactagtagggtattagagcatttattggtttctttcttccttgagttgagttacttctatcccaaaacagacaggtcttacgttaaccttcaccttaaaaaggatattgccttttttccttctgcttcacaaatttgctccaacactaattcccttgcatttgatttcatcattatgattgttcatcaaccattcttcacgaaaaatcctcacatcacgtcatataaaggttttattatttttaatggaaaataacattgagagattattttcctttttcataaaatttccctttttccctccctctattctcttcactaacattggaaattaaacaagttttacaggctctattgttattaaccctcaaatctattcttcagagaatatatgatcagcctgtgtttactatgcacctactcttataccagaaactgtgaatacaaatacatatatttcctacttctctctttaattggggcacacagtcccatggtccattctgcatttttcaaaattgaatataattgcaacacctgataggttttgccacttagaattctgaaaataaatcaactctgttctaaaccttaagtttccactattattgccaaactctggactgatacaataagccttttaaaaggtgaatttaacacttcacctattatggttatttaagtaaaatgagttttcaaatatttcccccaggagaaatataaaggactatgcaaaaatagaaggagaaaaaggttagttagaggtccagaaggagaattaaaacatggaattgataaattcacctactgatttatgatgttaatgagcaggtacctatacattttatttacttgaaaacattttggccattaactatttctgttttctaaatacatttgtgtttataaaattttaaaatctatatcacaagacttagacatgcctttctttgcaataaagtatatatacctttttacaaatgtaaattttattttgaaacttggcattttctttataattcctaagatatctttgggcacacagactacagataaatctatttaaatcaattcatcattatcactggtttaaatatttaatgtaaccattggaatttttattcttattccttcagagaattctccttttcttctatgtttctgactgcaatacagcccgatattgttattccctatatcttaatgaaaagcaagctgtcagcaaaatgcatttatagtttccagatgtttaacaatctctttcttcttttcttttttgttattatttttatttatgtgaagttagttagctgaggtaccaaaaaagagacaaagacagtattaaaaagtgtgcataaagcacttatttttgtactttagtacaatgttttatagaactttgtgatctatctaaatatattaagtaaaattacaccattcacttgttgggaaaataatctttggtttggaagatattaacataatgggcatcttagaatcataaatcacatgaaatgagagacaatgcaatattgtataattcctggatgatgcaattgttttaattgaattttcaagtgccattataaagttttaaaaattatcaa

SEQ ID NO: 51 - RNA, 9364 nt, Homo sapiens
  source 1..9364: mol_type = mRNA
ataatcaaggaaaccttttccgggtggggatctctgaaattactcagataacagtgctgtgccaaaaacctgtggattttctctacaaaaattattgagcaaccctaattaacctgattttttgctgataatcactctcaatggaatcaaatcacaaatccggggatggattgagcggcactcagaaggaagcagccctccgcgcactggtccagcgcacaggatatagcttggtccaggaatttagaggttcctatagatggaagcagtgtgtctgggcacatgcccagagccagaagcgagcatgagcacagcaattcctggcctcaaaaaaggaaataatgctttgcagtccatcattttgcaaactttgctggaaaaggaaaatggacaaagaaaatatggtggccctccacctggttgggatgctgcaccccctgaaaggggctgtgaaatttttattggaaaacttccccgagacctttttgaggatgagcttataccattatgtgaaaaaatcggtaaaatttatgaaatgagaatgatgatggattttaatggcaacaatagaggatatgcatttgtaacattttcaaataaagtggaagccaagaatgcaatcaagcaacttaataattatgaaattagaaatgggcgcctcttaggggtttgtgccagtgtggacaactgccgattatttgttgggggcatcccaaaaaccaaaaagagagaagaaatcttatcggagatgaaaaaggttactgaaggtgttgtcgatgtcatcgtctacccaagcgctgcagataaaaccaaaaaccgaggctttgccttcgtggagtatgagagtcatcgagcagctgccatggcgaggaggaaactgctaccaggaagaattcagttatggggacatggtattgcagtagactgggcagagccagaagtagaagttgatgaagatacaatgtcttcagtgaaaatcctatatgtaagaaatcttatgctgtctacctctgaagagatgattgaaaaggaattcaacaatatcaaaccaggtgctgtggagagggtgaagaaaattcgagactatgcttttgtgcacttcagtaaccgagaagatgcagttgaggctatgaaagctttaaatggcaaggtgctggatggttcccccattgaagtcaccctagcaaaaccagtggacaaggacagttatgttaggtatacccgaggcacaggtggaaggggcaccatgctgcaaggagagtatacctactctttgggccaagtttatgatcccaccacaacctaccttggagctcctgtcttctatgccccccagacctatgcagcaattcccagtcttcatttcccagccaccaaaggacatctcagcaacagagccattatccgagccccttctgttagaggggctgcgggagtgagaggactgggcggccgtggctatttggcatacacaggcctgggtcgaggataccaggtcaaaggagacaaaagagaagacaaactctatgacattttacctgggatggagctcaccccaatgaatcctgtcacattaaaaccccaaggaattaaactcgctccccagatattagaagagatttgtcagaaaaataactggggacagccagtgtaccagctgcactctgctattggacaagaccaaagacagctattcttgtacaaaataactattcctgctctagccagccagaatcctgcaatccaccctttcacacctccaaagctgagtgcctttgtggatgaagcaaagacgtatgcagccgaatacaccctgcagaccctgggcatccccactgatggaggcgatggcaccatggctactgctgctgctgctgctactgctttcccaggatatgctgtccctaatgcaactgcacccgtgtctgcagcccagctcaagcaagcggtaacccttggacaagacttagcagcatatacaacctatgaggtctacccaacttttgcagtgactgcccgaggggatggatatggcaccttctgaagatgcttttttaaatttaagaataagacacacaaaactctattaaaaaaaaaaaagaaataaacctctaactcggtccccaatgatcataaataatatgtttcctaaagaaatgcctttccagagactgtatagcttataccaattatagaatcatgaagtaagaacgttattctgaaatgaaatcaaatgttaaagagccttttgtttaacagaaagctggagagccactgccccaaaagttgttcttccagtcttcctgagtgtttggggagtatgtttacaaatcctgccagggtgaagagagatcttgtagctggcaagatttgggagggatggagcagacctgggcagtcagtaataatcaagggaagccaagtaaactccagagtcttacctctggctcacagctgcttttgtgatacacaaagtcacaaagaggaaaagggaagggaggaggttgcttttcaaaatgcattcaccatggctcactgtgtggggcatgattgcaggcctgagctcatgaaagactgtccaagattttgtggtttcaacctctcttcagcctgaaaattccatccagtcgatttgtcacagatcaggatcttcctgtcccctgcagcccatggggacttcctagaaatagatatgatatgttgtagatgtggttaaaacaaatccaggccccattgtttattcagctcattagtccacacctatttttccctagaactcctaccttttccaataatcatgccagcatgaaatgaccttttctactttaaaattttttgacttgttcatattgcttgattttcatcatctcagactcatttttctcctacctaatatcataaaaaagtaaatcagatatacagaatatcatttatgatacttcttcctttcagcaaattgactgaaaagtcattttaaagagagaagtgcagatttgctctcatttctttgatattgttgcagtatccaccccaccatctagcaatcagagatgcttatattaacttgggattagctttgcattatctaacccatttattttaaatctgccaggaaatcctctaactttccttcctttttgtttcagtaagtatcaggcagcttcaccatacctgagtccttttgtcttgaagctgccacagaaaaatcttacagcaatcattgctgattagaaactgtttcagacaatcagcatgggtgttatttaccaaattccccccagagtcctaggcctcttctccagaaatatctgatgatgaagtgaggggagggcaacggtgctacaaaacacggaacagaggtaaagagaaggcactactttcttgccatacttgtaaatgattgctttgttcaaacataaataatcttaagtccaacaccaaatacctgttactcctacatcaatctcattagtggtttaagacacagtactagaattttcattttttaaaatcccttggcccttaaaaaaatttacagaacacaaaggaaaacataaacacaaagacatggaaaattttgtcaactccttaatggaattctgtgatcaaaaagcaggccagattctaatcaaaatcaggtaaattttaatcacaatcagaagtacttgtaacatttcagttgtcctaactccaatgagataacaaagcctccaaggctacagctgaaactctgaaaggccctgtgctttctactttacatttagcgtctaatatttcctaggacagtagttcccaaagtaggctgtacattagaatctcctggagagctttttaaatgctaatgccaataacatatctcataaaatttacactagaatttccttggatgggtgcctggcatcagtattttttaatgatccctaggtgatttcaatgtgcacctaggtttgggaattgctatattcttgtatttatactctatctctttatttcttttcctttttatttcacttttagtaaatagaaaaatgcaattaaaaaatcatctaagttaagacaaatagccaaatcatatttatcccataaatagtatgaatttaaatgtaacttacgtaggcttcaaactaactctaaaaatgagaaagtaaaacaatcttgtcctaaaacaattcttagatccaaatttacactcccctacagatctgcttttattctcaaagacagtgattacattgacataacaaatgtccattggggcaggcaaatttctactgagtctaaaaagcattccaaattctaggagcaaaagttctgattttccttaaaatctttgcaaataaacagcaaagggtattctggatgtcttgtcaatgcaaattaggtttgatttagagaagttttaaatttaatcgcaatgaaaagattagttaatacatgaatctttttatgagaaaaaatcattattctggataaagtcataattgtttgtttacaatattaagagcagacacctaaacatacaataataccaaagtattgagaaaattaccactggaacaccatctcaagttatcacaccatttacctctttagagccagttttgtataagtaggtagaagtggttttggtttttttgctaaaattttactttgttttagtaatatctagggaatactttgaattccattaaaataccttcatagaaagtgttaatgtgtctaccatgccatatgtttatttttactttccccaagttaggtagatagagcaagtcctgctaccataagtttaaaattattaagacaacgggcttttttaaattgctggtagaaccaaaggctaattttgcataggtttatatttttagacttaagaaattagcacctatataataccttcataccttctgcatcactatcaatttttctgtaatttcttatttgaatgcttcctaattctttacagaaaaaattaaaatttgtcacccaaccaggtgataataacaaaacaataatttaggaacatccagtaaatttgaattagttctctaaattttggtaattaatagcttattgataattaaaatcagcctaatgttgcttaagtaggaggaaattgatcagcaatgggattgggtatctattttctttcctataaaagtgctgcctcttctcaaattgctttatttctctcccactgcattagtatatctagaacagcaccttcaactgcaacaaaatataattctcctagatgactaggatatcctccaatctggtatcatgccaaacaacactatgtttggggaacagcacccgaaatgctgctaaaaagctacctcatgtcaagaaaatatggatttcaaaaaatatctggcacagtggcccatgcctataaccccagtactttgggaggccaaggcaggaggattgctggaggccaggagttcaagaccagcctgggcaacatggagagactccgcctctacaaaaaaaaaaaaaacaaatttaatttaattggccagttgtggtggcgtgggcctgtggtcccagctacttgggaagctaaggcaggaggattgctggaggcctggagggcaaggctgcagtgagccatgttcacatcgctgcactccagcctgggtgacagagcaagaccctgtctccaaaaaagcaaaaataaatatctaacaaaaaggaacctgtttccaaaccacagtttcttaactttttcatgatggaatagaaattaaactataagtatatactgataatagctgtgcaggaaattggtttttattttacttaaaagtattttttagaaaaaatgttattctgattgcatagattagcaaaatattgcatcaatataatgttccatttttattaatttttgttatattctaaatgaccgttttataattctgtcttggacaactgggccacagttagaatccacatagacagggaggttcttaggtgtgccatttgtagaataaagggcaatcaagttaatattcggggtaaattttcattgcattctaacctatgaagtttgataaagtatattattattgaacaactctaacactaagatttcctaaaagtaacttacaacaaaaaatgaactatgtgctcaatattcacataaagtcatgattattctcttcttgcattcatataaaagttcccagggggaagaaaatatatctaaaaattagactagaaacttggcactttttatgtattgtagatacactttcatatattgtaaatacatctgttaaagataaaactaagtttctaacatagcactacaaggaaactttggataaatatttttaaaaagaaaatgttaataggataacttaagattttgtatatacttattggaagacaggaagaaactaggcagtaaatattcctacctgacgtgcaaccattgtcaatcaggttttgttaaactgtcaaatttgattttgaaattttagtttgcccaaataatatcttgaaaatgctctgaattttactagaacatattcatagaaagtgtaatgtatctaccaggctctatgtttgtgtttgccaacattagaaaaacaaagtaagttgcgctataatgagtgaaactaaggaaaatgagatttttctgactcccattccctctcttaaaaagtaaaatatttaagctttttttattaaaggaaaaatgggtacagaatactctattgccctccaaatgtccaacatttttaaagccatgttgtccaacaatgtttatacagaaaatagaatgcataggtttaatatagatagcaatgtatatgtaggctcatttgtgggagattgcaaactttatgtgcccccagttctccttggttttgtaggaaagctaaaagaaaaccttgtactctcactaataatgaaattcatagccaatttagaaagggaaaataacctgaaaaccaaaatacctcattcatctgaagatgcctggaataaaatatggaattcttctgtacctacctcccccttacagacttttctcctattttgagagagaaattcaagaagaagaatacagcttgttctctttccacaaatgatactttgtctaagggaataggagataccaagaaacagtatctttaaaaaagtaagctgcccacaggccttcttggagaatgaagaatatcagccccattcagggtttaccaatgtctctctgaggtgacaatacattctggttttccaggacagtcctggcttaggtctgctgtccttctataaatattaacaatgatccctcttatttccaaacgtgtcccagtttagacaacaaattatacagtgtctgcccccaacacacacacacacaccccaccatctttatgggttgttgttgttttcttgttgtttttaattttgtagaattggctttgtcttctccaaagcagataacagcttaaactaaagagcctagctacttctttctgactgagcagggtcatagtaaacagagaagaatggatcacttcctcctctgaaatctccatcaatccactttctgtccaactatctctctacccagcctatttttggtcctagttttcctctcataacccctgggctgtgagtcctgagttctgccttcatatggcagagcaagctagtcattattatgccaggttaggtggtggggcaagacaaaagatagctcttggggacacttgcttggtctgtagagatccacagggcactagtagggtattagagcatttattggtttctttcttccttgagttgagttacttctatcccaaaacagacaggtcttacgttaaccttcaccttaaaaaggatattgccttttttccttctgcttcacaaatttgctccaacactaattcccttgcatttgatttcatcattatgattgttcatcaaccattcttcacgaaaaatcctcacatcacgtcatataaaggttttattatttttaatggaaaataacattgagagattattttcctttttcataaaatttccctttttccctccctctattctcttcactaacattggaaattaaacaagttttacaggctctattgttattaaccctcaaatctattcttcagagaatatatgatcagcctgtgtttactatgcacctactcttataccagaaactgtgaatacaaatacatatatttcctacttctctctttaattggggcacacagtcccatggtccattctgcatttttcaaaattgaatataattgcaacacctgataggttttgccacttagaattctgaaaataaatcaactctgttctaaaccttaagtttccactattattgccaaactctggactgatacaataagccttttaaaaggtgaatttaacacttcacctattatggttatttaagtaaaatgagttttcaaatatttcccccaggagaaatataaaggactatgcaaaaatagaaggagaaaaaggttagttagaggtccagaaggagaattaaaacatggaattgataaattcacctactgatttatgatgttaatgagcaggtacctatacattttatttacttgaaaacattttggccattaactatttctgttttctaaatacatttgtgtttataaaattttaaaatctatatcacaagacttagacatgcctttctttgcaataaagtatatatacctttttacaaatgtaaattttattttgaaacttggcattttctttataattcctaagatatctttgggcacacagactacagataaatctatttaaatcaattcatcattatcactggtttaaatatttaatgtaaccattggaatttttattcttattccttcagagaattctccttttcttctatgtttctgactgcaatacagcccgatattgttattccctatatcttaatgaaaagcaagctgtcagcaaaatgcatttatagtttccagatgtttaacaatctctttcttcttttcttttttgttattatttttatttatgtgaagttagttagctgaggtaccaaaaaagagacaaagacagtattaaaaagtgtgcataaagcacttatttttgtactttagtacaatgttttatagaactttgtgatctatctaaatatattaagtaaaattacaccattcacttgttgggaaaataatctttggtttggaagatattaacataatgggcatcttagaatcataaatcacatgaaatgagagacaatgcaatattgtataattcctggatgatgcaattgttttaattgaattttcaagtgccattataaagttttaaaaattatcaa

SEQ ID NO: 52 - RNA, 9173 nt, Homo sapiens
  source 1..9173: mol_type = mRNA
ataatcaaggaaaccttttccgggtggggatctctgaaattactcagtgagcaaccctaattaacctgattttttgctgataatcactctcaatggaatcaaatcacaaatccggggatggattgagcggcactcagaaggaagcagccctccgcgcactggtccagcgcacaggatatagcttggtccaggaaaatggacaaagaaaatatggtggccctccacctggttgggatgctgcaccccctgaaaggggctgtgaaatttttattggaaaacttccccgagacctttttgaggatgagcttataccattatgtgaaaaaatcggtaaaatttatgaaatgagaatgatgatggattttaatggcaacaatagaggatatgcatttgtaacattttcaaataaagtggaagccaagaatgcaatcaagcaacttaataattatgaaattagaaatgggcgcctcttaggggtttgtgccagtgtggacaactgccgattatttgttgggggcatcccaaaaaccaaaaagagagaagaaatcttatcggagatgaaaaaggttactgaaggtgttgtcgatgtcatcgtctacccaagcgctgcagataaaaccaaaaaccgaggctttgccttcgtggagtatgagagtcatcgagcagctgccatggcgaggaggaaactgctaccaggaagaattcagttatggggacatggtattgcagtagactgggcagagccagaagtagaagttgatgaagatacaatgtcttcagtgaaaatcctatatgtaagaaatcttatgctgtctacctctgaagagatgattgaaaaggaattcaacaatatcaaaccaggtgctgtggagagggtgaagaaaattcgagactatgcttttgtgcacttcagtaaccgagaagatgcagttgaggctatgaaagctttaaatggcaaggtgctggatggttcccccattgaagtcaccctagcaaaaccagtggacaaggacagttatgttaggtatacccgaggcacaggtggaaggggcaccatgctgcaaggagagtatacctactctttgggccaagtttatgatcccaccacaacctaccttggagctcctgtcttctatgccccccagacctatgcagcaattcccagtcttcatttcccagccaccaaaggacatctcagcaacagagccattatccgagccccttctgttagaggggctgcgggagtgagaggactgggcggccgtggctatttggcatacacaggcctgggtcgaggataccaggtcaaaggagacaaaagagaagacaaactctatgacattttacctgggatggagctcaccccaatgaatcctgtcacattaaaaccccaaggaattaaactcgctccccagatattagaagagatttgtcagaaaaataactggggacagccagtgtaccagctgcactctgctattggacaagaccaaagacagctattcttgtacaaaataactattcctgctctagccagccagaatcctgcaatccaccctttcacacctccaaagctgagtgcctttgtggatgaagcaaagacgtatgcagccgaatacaccctgcagaccctgggcatccccactgatggaggcgatggcaccatggctactgctgctgctgctgctactgctttcccaggatatgctgtccctaatgcaactgcacccgtgtctgcagcccagctcaagcaagcggtaacccttggacaagacttagcagcatatacaacctatgaggtctacccaacttttgcagtgactgcccgaggggatggatatggcaccttctgaagatgcttttttaaatttaagaataagacacacaaaactctattaaaaaaaaaaaagaaataaacctctaactcggtccccaatgatcataaataatatgtttcctaaagaaatgcctttccagagactgtatagcttataccaattatagaatcatgaagtaagaacgttattctgaaatgaaatcaaatgttaaagagccttttgtttaacagaaagctggagagccactgccccaaaagttgttcttccagtcttcctgagtgtttggggagtatgtttacaaatcctgccagggtgaagagagatcttgtagctggcaagatttgggagggatggagcagacctgggcagtcagtaataatcaagggaagccaagtaaactccagagtcttacctctggctcacagctgcttttgtgatacacaaagtcacaaagaggaaaagggaagggaggaggttgcttttcaaaatgcattcaccatggctcactgtgtggggcatgattgcaggcctgagctcatgaaagactgtccaagattttgtggtttcaacctctcttcagcctgaaaattccatccagtcgatttgtcacagatcaggatcttcctgtcccctgcagcccatggggacttcctagaaatagatatgatatgttgtagatgtggttaaaacaaatccaggccccattgtttattcagctcattagtccacacctatttttccctagaactcctaccttttccaataatcatgccagcatgaaatgaccttttctactttaaaattttttgacttgttcatattgcttgattttcatcatctcagactcatttttctcctacctaatatcataaaaaagtaaatcagatatacagaatatcatttatgatacttcttcctttcagcaaattgactgaaaagtcattttaaagagagaagtgcagatttgctctcatttctttgatattgttgcagtatccaccccaccatctagcaatcagagatgcttatattaacttgggattagctttgcattatctaacccatttattttaaatctgccaggaaatcctctaactttccttcctttttgtttcagtaagtatcaggcagcttcaccatacctgagtccttttgtcttgaagctgccacagaaaaatcttacagcaatcattgctgattagaaactgtttcagacaatcagcatgggtgttatttaccaaattccccccagagtcctaggcctcttctccagaaatatctgatgatgaagtgaggggagggcaacggtgctacaaaacacggaacagaggtaaagagaaggcactactttcttgccatacttgtaaatgattgctttgttcaaacataaataatcttaagtccaacaccaaatacctgttactcctacatcaatctcattagtggtttaagacacagtactagaattttcattttttaaaatcccttggcccttaaaaaaatttacagaacacaaaggaaaacataaacacaaagacatggaaaattttgtcaactccttaatggaattctgtgatcaaaaagcaggccagattctaatcaaaatcaggtaaattttaatcacaatcagaagtacttgtaacatttcagttgtcctaactccaatgagataacaaagcctccaaggctacagctgaaactctgaaaggccctgtgctttctactttacatttagcgtctaatatttcctaggacagtagttcccaaagtaggctgtacattagaatctcctggagagctttttaaatgctaatgccaataacatatctcataaaatttacactagaatttccttggatgggtgcctggcatcagtattttttaatgatccctaggtgatttcaatgtgcacctaggtttgggaattgctatattcttgtatttatactctatctctttatttcttttcctttttatttcacttttagtaaatagaaaaatgcaattaaaaaatcatctaagttaagacaaatagccaaatcatatttatcccataaatagtatgaatttaaatgtaacttacgtaggcttcaaactaactctaaaaatgagaaagtaaaacaatcttgtcctaaaacaattcttagatccaaatttacactcccctacagatctgcttttattctcaaagacagtgattacattgacataacaaatgtccattggggcaggcaaatttctactgagtctaaaaagcattccaaattctaggagcaaaagttctgattttccttaaaatctttgcaaataaacagcaaagggtattctggatgtcttgtcaatgcaaattaggtttgatttagagaagttttaaatttaatcgcaatgaaaagattagttaatacatgaatctttttatgagaaaaaatcattattctggataaagtcataattgtttgtttacaatattaagagcagacacctaaacatacaataataccaaagtattgagaaaattaccactggaacaccatctcaagttatcacaccatttacctctttagagccagttttgtataagtaggtagaagtggttttggtttttttgctaaaattttactttgttttagtaatatctagggaatactttgaattccattaaaataccttcatagaaagtgttaatgtgtctaccatgccatatgtttatttttactttccccaagttaggtagatagagcaagtcctgctaccataagtttaaaattattaagacaacgggcttttttaaattgctggtagaaccaaaggctaattttgcataggtttatatttttagacttaagaaattagcacctatataataccttcataccttctgcatcactatcaatttttctgtaatttcttatttgaatgcttcctaattctttacagaaaaaattaaaatttgtcacccaaccaggtgataataacaaaacaataatttaggaacatccagtaaatttgaattagttctctaaattttggtaattaatagcttattgataattaaaatcagcctaatgttgcttaagtaggaggaaattgatcagcaatgggattgggtatctattttctttcctataaaagtgctgcctcttctcaaattgctttatttctctcccactgcattagtatatctagaacagcaccttcaactgcaacaaaatataattctcctagatgactaggatatcctccaatctggtatcatgccaaacaacactatgtttggggaacagcacccgaaatgctgctaaaaagctacctcatgtcaagaaaatatggatttcaaaaaatatctggcacagtggcccatgcctataaccccagtactttgggaggccaaggcaggaggattgctggaggccaggagttcaagaccagcctgggcaacatggagagactccgcctctacaaaaaaaaaaaaaacaaatttaatttaattggccagttgtggtggcgtgggcctgtggtcccagctacttgggaagctaaggcaggaggattgctggaggcctggagggcaaggctgcagtgagccatgttcacatcgctgcactccagcctgggtgacagagcaagaccctgtctccaaaaaagcaaaaataaatatctaacaaaaaggaacctgtttccaaaccacagtttcttaactttttcatgatggaatagaaattaaactataagtatatactgataatagctgtgcaggaaattggtttttattttacttaaaagtattttttagaaaaaatgttattctgattgcatagattagcaaaatattgcatcaatataatgttccatttttattaatttttgttatattctaaatgaccgttttataattctgtcttggacaactgggccacagttagaatccacatagacagggaggttcttaggtgtgccatttgtagaataaagggcaatcaagttaatattcggggtaaattttcattgcattctaacctatgaagtttgataaagtatattattattgaacaactctaacactaagatttcctaaaagtaacttacaacaaaaaatgaactatgtgctcaatattcacataaagtcatgattattctcttcttgcattcatataaaagttcccagggggaagaaaatatatctaaaaattagactagaaacttggcactttttatgtattgtagatacactttcatatattgtaaatacatctgttaaagataaaactaagtttctaacatagcactacaaggaaactttggataaatatttttaaaaagaaaatgttaataggataacttaagattttgtatatacttattggaagacaggaagaaactaggcagtaaatattcctacctgacgtgcaaccattgtcaatcaggttttgttaaactgtcaaatttgattttgaaattttagtttgcccaaataatatcttgaaaatgctctgaattttactagaacatattcatagaaagtgtaatgtatctaccaggctctatgtttgtgtttgccaacattagaaaaacaaagtaagttgcgctataatgagtgaaactaaggaaaatgagatttttctgactcccattccctctcttaaaaagtaaaatatttaagctttttttattaaaggaaaaatgggtacagaatactctattgccctccaaatgtccaacatttttaaagccatgttgtccaacaatgtttatacagaaaatagaatgcataggtttaatatagatagcaatgtatatgtaggctcatttgtgggagattgcaaactttatgtgcccccagttctccttggttttgtaggaaagctaaaagaaaaccttgtactctcactaataatgaaattcatagccaatttagaaagggaaaataacctgaaaaccaaaatacctcattcatctgaagatgcctggaataaaatatggaattcttctgtacctacctcccccttacagacttttctcctattttgagagagaaattcaagaagaagaatacagcttgttctctttccacaaatgatactttgtctaagggaataggagataccaagaaacagtatctttaaaaaagtaagctgcccacaggccttcttggagaatgaagaatatcagccccattcagggtttaccaatgtctctctgaggtgacaatacattctggttttccaggacagtcctggcttaggtctgctgtccttctataaatattaacaatgatccctcttatttccaaacgtgtcccagtttagacaacaaattatacagtgtctgcccccaacacacacacacacaccccaccatctttatgggttgttgttgttttcttgttgtttttaattttgtagaattggctttgtcttctccaaagcagataacagcttaaactaaagagcctagctacttctttctgactgagcagggtcatagtaaacagagaagaatggatcacttcctcctctgaaatctccatcaatccactttctgtccaactatctctctacccagcctatttttggtcctagttttcctctcataacccctgggctgtgagtcctgagttctgccttcatatggcagagcaagctagtcattattatgccaggttaggtggtggggcaagacaaaagatagctcttggggacacttgcttggtctgtagagatccacagggcactagtagggtattagagcatttattggtttctttcttccttgagttgagttacttctatcccaaaacagacaggtcttacgttaaccttcaccttaaaaaggatattgccttttttccttctgcttcacaaatttgctccaacactaattcccttgcatttgatttcatcattatgattgttcatcaaccattcttcacgaaaaatcctcacatcacgtcatataaaggttttattatttttaatggaaaataacattgagagattattttcctttttcataaaatttccctttttccctccctctattctcttcactaacattggaaattaaacaagttttacaggctctattgttattaaccctcaaatctattcttcagagaatatatgatcagcctgtgtttactatgcacctactcttataccagaaactgtgaatacaaatacatatatttcctacttctctctttaattggggcacacagtcccatggtccattctgcatttttcaaaattgaatataattgcaacacctgataggttttgccacttagaattctgaaaataaatcaactctgttctaaaccttaagtttccactattattgccaaactctggactgatacaataagccttttaaaaggtgaatttaacacttcacctattatggttatttaagtaaaatgagttttcaaatatttcccccaggagaaatataaaggactatgcaaaaatagaaggagaaaaaggttagttagaggtccagaaggagaattaaaacatggaattgataaattcacctactgatttatgatgttaatgagcaggtacctatacattttatttacttgaaaacattttggccattaactatttctgttttctaaatacatttgtgtttataaaattttaaaatctatatcacaagacttagacatgcctttctttgcaataaagtatatatacctttttacaaatgtaaattttattttgaaacttggcattttctttataattcctaagatatctttgggcacacagactacagataaatctatttaaatcaattcatcattatcactggtttaaatatttaatgtaaccattggaatttttattcttattccttcagagaattctccttttcttctatgtttctgactgcaatacagcccgatattgttattccctatatcttaatgaaaagcaagctgtcagcaaaatgcatttatagtttccagatgtttaacaatctctttcttcttttcttttttgttattatttttatttatgtgaagttagttagctgaggtaccaaaaaagagacaaagacagtattaaaaagtgtgcataaagcacttatttttgtactttagtacaatgttttatagaactttgtgatctatctaaatatattaagtaaaattacaccattcacttgttgggaaaataatctttggtttggaagatattaacataatgggcatcttagaatcataaatcacatgaaatgagagacaatgcaatattgtataattcctggatgatgcaattgttttaattgaattttcaagtgccattataaagttttaaaaattatcaa

SEQ ID NO: 53 - RNA, 9197 nt, Homo sapiens
  source 1..9197: mol_type = mRNA
ataatcaaggaaaccttttccgggtggggatctctgaaattactcagtgagcaaccctaattaacctgattttttgctgataatcactctcaatggaatcaaatcacaaatccggggatggattgagcggcactcagaaggaagcagccctccgcgcactggtccagcgcacaggatatagcttggtccaggaaaatggacaaagaaaatatggtggccctccacctggttgggatgctgcaccccctgaaaggggctgtgaaatttttattggaaaacttccccgagacctttttgaggatgagcttataccattatgtgaaaaaatcggtaaaatttatgaaatgagaatgatgatggattttaatggcaacaatagaggatatgcatttgtaacattttcaaataaagtggaagccaagaatgcaatcaagcaacttaataattatgaaattagaaatgggcgcctcttaggggtttgtgccagtgtggacaactgccgattatttgttgggggcatcccaaaaaccaaaaagagagaagaaatcttatcggagatgaaaaaggttactgaaggtgttgtcgatgtcatcgtctacccaagcgctgcagataaaaccaaaaaccgaggctttgccttcgtggagtatgagagtcatcgagcagctgccatggcgaggaggaaactgctaccaggaagaattcagttatggggacatggtattgcagtagactgggcagagccagaagtagaagttgatgaagatacaatgtcttcagtgaaaatcctatatgtaagaaatcttatgctgtctacctctgaagagatgattgaaaaggaattcaacaatatcaaaccaggtgctgtggagagggtgaagaaaattcgagactatgcttttgtgcacttcagtaaccgagaagatgcagttgaggctatgaaagctttaaatggcaaggtgctggatggttcccccattgaagtcaccctagcaaaaccagtggacaaggacagttatgttaggtatacccgaggcacaggtggaaggggcaccatgctgcaaggagagtatacctactctttgggccaagtttatgatcccaccacaacctaccttggagctcctgtcttctatgccccccagacctatgcagcaattcccagtcttcatttcccagccaccaaaggacatctcagcaacagagccattatccgagccccttctgttagagaaatttacatgaatgtacctgtaggggctgcgggagtgagaggactgggcggccgtggctatttggcatacacaggcctgggtcgaggataccaggtcaaaggagacaaaagagaagacaaactctatgacattttacctgggatggagctcaccccaatgaatcctgtcacattaaaaccccaaggaattaaactcgctccccagatattagaagagatttgtcagaaaaataactggggacagccagtgtaccagctgcactctgctattggacaagaccaaagacagctattcttgtacaaaataactattcctgctctagccagccagaatcctgcaatccaccctttcacacctccaaagctgagtgcctttgtggatgaagcaaagacgtatgcagccgaatacaccctgcagaccctgggcatccccactgatggaggcgatggcaccatggctactgctgctgctgctgctactgctttcccaggatatgctgtccctaatgcaactgcacccgtgtctgcagcccagctcaagcaagcggtaacccttggacaagacttagcagcatatacaacctatgaggtctacccaacttttgcagtgactgcccgaggggatggatatggcaccttctgaagatgcttttttaaatttaagaataagacacacaaaactctattaaaaaaaaaaaagaaataaacctctaactcggtccccaatgatcataaataatatgtttcctaaagaaatgcctttccagagactgtatagcttataccaattatagaatcatgaagtaagaacgttattctgaaatgaaatcaaatgttaaagagccttttgtttaacagaaagctggagagccactgccccaaaagttgttcttccagtcttcctgagtgtttggggagtatgtttacaaatcctgccagggtgaagagagatcttgtagctggcaagatttgggagggatggagcagacctgggcagtcagtaataatcaagggaagccaagtaaactccagagtcttacctctggctcacagctgcttttgtgatacacaaagtcacaaagaggaaaagggaagggaggaggttgcttttcaaaatgcattcaccatggctcactgtgtggggcatgattgcaggcctgagctcatgaaagactgtccaagattttgtggtttcaacctctcttcagcctgaaaattccatccagtcgatttgtcacagatcaggatcttcctgtcccctgcagcccatggggacttcctagaaatagatatgatatgttgtagatgtggttaaaacaaatccaggccccattgtttattcagctcattagtccacacctatttttccctagaactcctaccttttccaataatcatgccagcatgaaatgaccttttctactttaaaattttttgacttgttcatattgcttgattttcatcatctcagactcatttttctcctacctaatatcataaaaaagtaaatcagatatacagaatatcatttatgatacttcttcctttcagcaaattgactgaaaagtcattttaaagagagaagtgcagatttgctctcatttctttgatattgttgcagtatccaccccaccatctagcaatcagagatgcttatattaacttgggattagctttgcattatctaacccatttattttaaatctgccaggaaatcctctaactttccttcctttttgtttcagtaagtatcaggcagcttcaccatacctgagtccttttgtcttgaagctgccacagaaaaatcttacagcaatcattgctgattagaaactgtttcagacaatcagcatgggtgttatttaccaaattccccccagagtcctaggcctcttctccagaaatatctgatgatgaagtgaggggagggcaacggtgctacaaaacacggaacagaggtaaagagaaggcactactttcttgccatacttgtaaatgattgctttgttcaaacataaataatcttaagtccaacaccaaatacctgttactcctacatcaatctcattagtggtttaagacacagtactagaattttcattttttaaaatcccttggcccttaaaaaaatttacagaacacaaaggaaaacataaacacaaagacatggaaaattttgtcaactccttaatggaattctgtgatcaaaaagcaggccagattctaatcaaaatcaggtaaattttaatcacaatcagaagtacttgtaacatttcagttgtcctaactccaatgagataacaaagcctccaaggctacagctgaaactctgaaaggccctgtgctttctactttacatttagcgtctaatatttcctaggacagtagttcccaaagtaggctgtacattagaatctcctggagagctttttaaatgctaatgccaataacatatctcataaaatttacactagaatttccttggatgggtgcctggcatcagtattttttaatgatccctaggtgatttcaatgtgcacctaggtttgggaattgctatattcttgtatttatactctatctctttatttcttttcctttttatttcacttttagtaaatagaaaaatgcaattaaaaaatcatctaagttaagacaaatagccaaatcatatttatcccataaatagtatgaatttaaatgtaacttacgtaggcttcaaactaactctaaaaatgagaaagtaaaacaatcttgtcctaaaacaattcttagatccaaatttacactcccctacagatctgcttttattctcaaagacagtgattacattgacataacaaatgtccattggggcaggcaaatttctactgagtctaaaaagcattccaaattctaggagcaaaagttctgattttccttaaaatctttgcaaataaacagcaaagggtattctggatgtcttgtcaatgcaaattaggtttgatttagagaagttttaaatttaatcgcaatgaaaagattagttaatacatgaatctttttatgagaaaaaatcattattctggataaagtcataattgtttgtttacaatattaagagcagacacctaaacatacaataataccaaagtattgagaaaattaccactggaacaccatctcaagttatcacaccatttacctctttagagccagttttgtataagtaggtagaagtggttttggtttttttgctaaaattttactttgttttagtaatatctagggaatactttgaattccattaaaataccttcatagaaagtgttaatgtgtctaccatgccatatgtttatttttactttccccaagttaggtagatagagcaagtcctgctaccataagtttaaaattattaagacaacgggcttttttaaattgctggtagaaccaaaggctaattttgcataggtttatatttttagacttaagaaattagcacctatataataccttcataccttctgcatcactatcaatttttctgtaatttcttatttgaatgcttcctaattctttacagaaaaaattaaaatttgtcacccaaccaggtgataataacaaaacaataatttaggaacatccagtaaatttgaattagttctctaaattttggtaattaatagcttattgataattaaaatcagcctaatgttgcttaagtaggaggaaattgatcagcaatgggattgggtatctattttctttcctataaaagtgctgcctcttctcaaattgctttatttctctcccactgcattagtatatctagaacagcaccttcaactgcaacaaaatataattctcctagatgactaggatatcctccaatctggtatcatgccaaacaacactatgtttggggaacagcacccgaaatgctgctaaaaagctacctcatgtcaagaaaatatggatttcaaaaaatatctggcacagtggcccatgcctataaccccagtactttgggaggccaaggcaggaggattgctggaggccaggagttcaagaccagcctgggcaacatggagagactccgcctctacaaaaaaaaaaaaaacaaatttaatttaattggccagttgtggtggcgtgggcctgtggtcccagctacttgggaagctaaggcaggaggattgctggaggcctggagggcaaggctgcagtgagccatgttcacatcgctgcactccagcctgggtgacagagcaagaccctgtctccaaaaaagcaaaaataaatatctaacaaaaaggaacctgtttccaaaccacagtttcttaactttttcatgatggaatagaaattaaactataagtatatactgataatagctgtgcaggaaattggtttttattttacttaaaagtattttttagaaaaaatgttattctgattgcatagattagcaaaatattgcatcaatataatgttccatttttattaatttttgttatattctaaatgaccgttttataattctgtcttggacaactgggccacagttagaatccacatagacagggaggttcttaggtgtgccatttgtagaataaagggcaatcaagttaatattcggggtaaattttcattgcattctaacctatgaagtttgataaagtatattattattgaacaactctaacactaagatttcctaaaagtaacttacaacaaaaaatgaactatgtgctcaatattcacataaagtcatgattattctcttcttgcattcatataaaagttcccagggggaagaaaatatatctaaaaattagactagaaacttggcactttttatgtattgtagatacactttcatatattgtaaatacatctgttaaagataaaactaagtttctaacatagcactacaaggaaactttggataaatatttttaaaaagaaaatgttaataggataacttaagattttgtatatacttattggaagacaggaagaaactaggcagtaaatattcctacctgacgtgcaaccattgtcaatcaggttttgttaaactgtcaaatttgattttgaaattttagtttgcccaaataatatcttgaaaatgctctgaattttactagaacatattcatagaaagtgtaatgtatctaccaggctctatgtttgtgtttgccaacattagaaaaacaaagtaagttgcgctataatgagtgaaactaaggaaaatgagatttttctgactcccattccctctcttaaaaagtaaaatatttaagctttttttattaaaggaaaaatgggtacagaatactctattgccctccaaatgtccaacatttttaaagccatgttgtccaacaatgtttatacagaaaatagaatgcataggtttaatatagatagcaatgtatatgtaggctcatttgtgggagattgcaaactttatgtgcccccagttctccttggttttgtaggaaagctaaaagaaaaccttgtactctcactaataatgaaattcatagccaatttagaaagggaaaataacctgaaaaccaaaatacctcattcatctgaagatgcctggaataaaatatggaattcttctgtacctacctcccccttacagacttttctcctattttgagagagaaattcaagaagaagaatacagcttgttctctttccacaaatgatactttgtctaagggaataggagataccaagaaacagtatctttaaaaaagtaagctgcccacaggccttcttggagaatgaagaatatcagccccattcagggtttaccaatgtctctctgaggtgacaatacattctggttttccaggacagtcctggcttaggtctgctgtccttctataaatattaacaatgatccctcttatttccaaacgtgtcccagtttagacaacaaattatacagtgtctgcccccaacacacacacacacaccccaccatctttatgggttgttgttgttttcttgttgtttttaattttgtagaattggctttgtcttctccaaagcagataacagcttaaactaaagagcctagctacttctttctgactgagcagggtcatagtaaacagagaagaatggatcacttcctcctctgaaatctccatcaatccactttctgtccaactatctctctacccagcctatttttggtcctagttttcctctcataacccctgggctgtgagtcctgagttctgccttcatatggcagagcaagctagtcattattatgccaggttaggtggtggggcaagacaaaagatagctcttggggacacttgcttggtctgtagagatccacagggcactagtagggtattagagcatttattggtttctttcttccttgagttgagttacttctatcccaaaacagacaggtcttacgttaaccttcaccttaaaaaggatattgccttttttccttctgcttcacaaatttgctccaacactaattcccttgcatttgatttcatcattatgattgttcatcaaccattcttcacgaaaaatcctcacatcacgtcatataaaggttttattatttttaatggaaaataacattgagagattattttcctttttcataaaatttccctttttccctccctctattctcttcactaacattggaaattaaacaagttttacaggctctattgttattaaccctcaaatctattcttcagagaatatatgatcagcctgtgtttactatgcacctactcttataccagaaactgtgaatacaaatacatatatttcctacttctctctttaattggggcacacagtcccatggtccattctgcatttttcaaaattgaatataattgcaacacctgataggttttgccacttagaattctgaaaataaatcaactctgttctaaaccttaagtttccactattattgccaaactctggactgatacaataagccttttaaaaggtgaatttaacacttcacctattatggttatttaagtaaaatgagttttcaaatatttcccccaggagaaatataaaggactatgcaaaaatagaaggagaaaaaggttagttagaggtccagaaggagaattaaaacatggaattgataaattcacctactgatttatgatgttaatgagcaggtacctatacattttatttacttgaaaacattttggccattaactatttctgttttctaaatacatttgtgtttataaaattttaaaatctatatcacaagacttagacatgcctttctttgcaataaagtatatatacctttttacaaatgtaaattttattttgaaacttggcattttctttataattcctaagatatctttgggcacacagactacagataaatctatttaaatcaattcatcattatcactggtttaaatatttaatgtaaccattggaatttttattcttattccttcagagaattctccttttcttctatgtttctgactgcaatacagcccgatattgttattccctatatcttaatgaaaagcaagctgtcagcaaaatgcatttatagtttccagatgtttaacaatctctttcttcttttcttttttgttattatttttatttatgtgaagttagttagctgaggtaccaaaaaagagacaaagacagtattaaaaagtgtgcataaagcacttatttttgtactttagtacaatgttttatagaactttgtgatctatctaaatatattaagtaaaattacaccattcacttgttgggaaaataatctttggtttggaagatattaacataatgggcatcttagaatcataaatcacatgaaatgagagacaatgcaatattgtataattcctggatgatgcaattgttttaattgaattttcaagtgccattataaagttttaaaaattatcaa

SEQ ID NO: 54 - DNA, 20 bp, synthetic construct
  misc_feature 1..20: note = Probe
  source 1..20: mol_type = other DNA
gaggcaggtcccctagaaga

SEQ ID NO: 55 - DNA, 21 bp, synthetic construct
  misc_feature 1..21: note = Primer
  source 1..21: mol_type = other DNA
ggagtgtggattcgcactcct

SEQ ID NO: 56 - DNA, 20 bp, synthetic construct
  misc_feature 1..20: note = Primer
  source 1..20: mol_type = other DNA
agattgagatcttctgcgac

SEQ ID NO: 57 - DNA, 19 bp, synthetic construct
  misc_feature 1..19: note = Primer
  source 1..19: mol_type = other DNA
ctgtgccttgggtggcttt

SEQ ID NO: 58 - DNA, 24 bp, synthetic construct
  misc_feature 1..24: note = Primer
  source 1..24: mol_type = other DNA
aaggaaagaagtcagaaggcaaaa

SEQ ID NO: 59 - DNA, 34 bp, synthetic construct
  misc_feature 1..34: note = Probe
  source 1..34: mol_type = other DNA
agctccaaattctttataagggtcgatgtccatg

SEQ ID NO: 60 - DNA, 11 bp, synthetic construct
  source 1..11: mol_type = other DNA
gcctatctgag

(19) United States
(12) Patent Application Publication
(10) Pub. No.: US 2024/0167040 A1
(43) Pub. Date: May 23, 2024

(54) ANTISENSE OLIGONUCLEOTIDE

(71) Applicant: Hoffmann-La Roche Inc., Little Falls, NJ (US)

(72) Inventors: Gitte Friis, Kobenhavn S (DK); Helene Maria Gylling, Kobenhavn S (DK); Dennis Jul Hansen, Farum (DK); Erich Koller, Basel (CH); Anaïs Irène Marie Lopez, Basel (CH); Dénia Mellal, Neuilly sur Marne (FR); Susanne Mohr, Basel (CH); Lykke Pedersen, Kobenhavn NV (DK); Johanna Marie Pose Vicente, Birsfelden (CH); Sabine Sewing, Basel-City (CH); Christian Weile, Farum (DK)

(21) Appl. No.: 18/170,685

(22) Filed: Feb. 17, 2023

(30) Foreign Application Priority Data
Feb. 21, 2022 (EP) 22157822.2
Jul. 18, 2022 (EP) 22185488.8

(51) Int. Cl. C12N 15/113; A61P 31/20

(52) U.S. Cl. CPC C12N 15/1137; A61P 31/20; C12N 2310/11; C12N 2310/351

(57) ABSTRACT

The present invention relates to antisense oligonucleotides that reduce expression of A1CF, as well as conjugates, salts and pharmaceutical compositions thereof. The invention also relates to uses of such antisense oligonucleotides, conjugates, salts and pharmaceutical compositions in methods for reducing A1CF expression and in medical uses and methods of treatment of disease, particularly treatment of hepatitis B virus (HBV) infection.

### CROSS-REFERENCE TO RELATED APPLICATIONS

This present application claims the benefit of European Patent application No. 22185488.8 filed on Jul. 18, 2022, which claims priority from European Patent application No. 22157822.2 filed on Feb. 21, 2022, which is incorporated hereby in its entirety.

### INCORPORATION BY REFERENCE OF SEQUENCE LISTING PROVIDED ELECTRONICALLY

This application contains a Sequence Listing submitted as an electronic text file named “P37373-WO-US.xml”, having a size of 263,832 bytes, and created on Nov. 13, 2023. The information contained in this electronic file is hereby incorporated by reference in its entirety. No new matter is contained in Sequence Listing

### FIELD OF THE INVENTION

The present invention relates to antisense oligonucleotides that reduce expression of A1CF, as well as conjugates, salts and pharmaceutical compositions thereof. The invention also relates to uses of such antisense oligonucleotides, conjugates, salts and pharmaceutical compositions in methods for reducing A1CF expression and in medical uses and methods of treatment of disease, particularly treatment of hepatitis B virus (HBV) infection.

### BACKGROUND

Hepatitis B is an infectious disease caused by the hepatitis B virus (HBV), a small hepatotropic virus that replicates through reverse transcription. Chronic HBV infection is a key factor for severe liver diseases such as liver cirrhosis and hepatocellular carcinoma. Current treatments for chronic HBV infection are based on administration of pegylated type 1 interferons or nucleos(t)ide analogues, such as lamivudine, adefovir, entecavir, tenofovir disoproxil, and tenofovir alafenamide, which target the viral polymerase, a multifunctional reverse transcriptase. Treatment success is usually measured as loss of hepatitis B surface antigen (HBsAg). However, a complete HBsAg clearance is rarely achieved since Hepatitis B virus DNA persists in the body after infection.

HBV persistence is mediated by an episomal form of the HBV genome which is stably maintained in the nucleus. This episomal form is called “covalently closed circular DNA” (cccDNA). HBV is an incomplete double-stranded circular DNA virus. After HBV infects a cell, the circular DNA is transferred into the nucleus and is repaired to cccDNA by DNA polymerase. The cccDNA serves as a template for all HBV transcripts, including pregenomic RNA (pgRNA).

pgRNA serves as a template for HBV genomic DNA, and is thus a viral replicative intermediate critical for viral replication. The presence of a few copies of cccDNA might be sufficient to reinitiate a full-blown HBV infection. Current treatments for HBV do not target cccDNA. A cure of chronic HBV infection, however, would require the elimination of cccDNA (reviewed by Nassal, Gut. 2015 December; 64(12):1972-84. doi: 10.1136/gutjnl-2015-309809).

A1CF (APOBEC1 complementation factor) is a component of the apolipoprotein B mRNA editing enzyme complex which is responsible for the posttranscriptional editing of a CAA codon for Gln to a UAA codon for stop in apolipoprotein B mRNA. The introduction of a stop codon into apolipoprotein B mRNA alters lipid metabolism in the gastrointestinal tract. The editing enzyme complex comprises a minimal core composed of the cytidine deaminase APOBEC1 (apolipoprotein B mRNA editing enzyme catalytic subunit 1) and a complementation factor encoded by the A1CF gene. The A1CF protein has three non-identical RNA recognition motifs and belongs to the hnRNP R family of RNA-binding proteins. It binds to apolipoprotein B mRNA and is probably responsible for docking the catalytic subunit, APOBEC1, to the mRNA to allow it to deaminate its target cytosine (see Chester et al., EMBO J. 2003 Aug. 1; 22(15):3971-82).

Many reports on the apolipoprotein B mRNA editing enzyme complex are focused on the cytidine deaminase APOBEC1, rather than on the APOBEC1 complementation factor. It has been shown that APOBEC1 does not only edit apolipoprotein B mRNA, but also viral genomes including HBV.

In a mouse model for HBV replication, Renard et al. showed that mouse APOBEC1 edited HBV in vivo (Renard et al., J Mol Biol. 2010 Jul. 16; 400(3):323-34. doi: 10.1016/j.jmb.2010.05.029). In contrast, rat APOBEC1 did not inhibit HBV DNA production (Rösler et al., Hepatology. 2005 August; 42(2):301-9).

Gonzalez et al. showed that human APOBEC1 edits HBV DNA. In cells co-transfected with HBV and human APOBEC1, several G to A hypermutations were identified in the HBV genome. Further, the presence of human APOBEC1 impacted replication of HBV DNA. Specifically, it was shown that an increased expression of APOBEC1 resulted in a decreased amount of HBV DNA (Gonzalez et al., Retrovirology. 2009 Oct. 21; 6:96. doi: 10.1186/1742-4690-6-96).

To our knowledge A1CF has never been identified as a cccDNA dependency factor in the context of cccDNA stability and maintenance, nor have molecules inhibiting A1CF ever been suggested as cccDNA destabilizers for the treatment of HBV infection.

Furthermore, to our knowledge the only disclosure of oligonucleotides potentially related to the regulation of A1CF expression are suggested in WO 2016/142948. However, WO 2016/142948 relates to the alteration of splicing of a number of listed targets including A1CF, to produce alternative splice variants. The oligonucleotides are however decoy oligonucleotides encoding splicing-factor binding sites and therefore do not bind to the targets as such. WO 2016/142948 also mentions a list of treatments including cancer, inflammation, immunological disorders, neurodegeneration, Alzheimer disease, Parkinson, viral infections (HIV, HSV, HBV). There are however no specific examples of oligonucleotides targeting A1CF nor their use in HBV.

### SUMMARY OF THE INVENTION

HBV cccDNA in infected hepatocytes is responsible for persistent chronic infection and reactivation, being the template for all viral subgenomic transcripts and pre-genomic RNA (pgRNA) to ensure both newly synthesized viral progeny and cccDNA pool replenishment via intracellular nucleocapsid recycling. A1CF is associated with cccDNA stability. Inhibiting A1CF leads to destabilization of cccDNA in HBV infected subjects, which in turn opens the opportunity for a complete cure of chronically infected HBV patients.

Accordingly, the present invention relates to antisense oligonucleotides capable of reducing expression of A1CF. Reduction of A1CF expression may reduce cccDNA levels, which may be useful in treatment of HBV infection, particularly chronic HBV infection.

The invention provides an antisense oligonucleotide of 12 to 30 nucleotides in length, which comprises a contiguous nucleotide sequence of 12 to 30 nucleotides in length, wherein the contiguous nucleotide sequence is capable of binding to a target sequence in an apolipoprotein B mRNA editing enzyme catalytic subunit 1 (APOBEC1) Complementation Factor (A1CF) mRNA, wherein the target sequence is a sequence of at least 17 contiguous nucleotides within any of the following sequences:

| | (positions 2181-2199 of SEQ ID NO 45) |
|---|---|
| (SEQ ID NO 32) | |
| | GCCUAUCUGAGAAACUUUU, |
| | (positions 6951-6970 of SEQ ID NO 45) |
| (SEQ ID NO 42) | |
| | GAGAAAAACCUAUAAUGCCU, |
| | (positions 16970-16989 of SEQ ID NO 45) |
| (SEQ ID NO 43) | |
| | AAGUAAAAUUAACAUGUCCA, |
| | (positions 26358-26377 of SEQ ID NO 45) |
| (SEQ ID NO 39) | |
| | AAACACCACAAUCUUAAAAC, |
| | (positions 38053-38071 of SEQ ID NO 45) |
| (SEQ ID NO 34) | |
| | CAGGUAUAUAACAAGUUCA, |
| | and |
| | (positions 78973-78990 of SEQ ID NO 45) |
| (SEQ ID NO 44) | |
| | AGACACACAAAACUCUAU, |

and wherein the antisense oligonucleotide is capable of reducing A1CF expression.

In some embodiments, the target sequence is selected from the following sequences:

| | (positions 6953-6970 of SEQ ID NO 45) |
|---|---|
| (SEQ ID NO 31) | |
| | GAAAAACCUAUAAUGCCU, |
| | (positions 2181-2199 of SEQ ID NO 45) |
| (SEQ ID NO 32) | |
| | GCCUAUCUGAGAAACUUUU, |
| | (positions 16970-16988 of SEQ ID NO 45) |
| (SEQ ID NO 33) | |
| | AAGUAAAAUUAACAUGUCC, |
| | (positions 38053-38071 of SEQ ID NO 45) |
| (SEQ ID NO 34) | |
| | CAGGUAUAUAACAAGUUCA, |
| | (positions 78973-78989 of SEQ ID NO 45) |
| (SEQ ID NO 35) | |
| | AGACACACAAAACUCUA, |
| | (positions 6952-6968 of SEQ ID NO 45) |
| (SEQ ID NO 36) | |
| | AGAAAAACCUAUAAUGC, |
| | (positions 6953-6969 of SEQ ID NO 45) |
| (SEQ ID NO 37) | |
| | GAAAAACCUAUAAUGCC, |
| | (positions 16972-16989 of SEQ ID NO 45) |
| (SEQ ID NO 38) | |
| | GUAAAAUUAACAUGUCCA, |
| | (positions 26358-26377 of SEQ ID NO 45) |
| (SEQ ID NO 39) | |
| | AAACACCACAAUCUUAAAAC, |
| | (positions 78974-78990 of SEQ ID NO 45) |
| (SEQ ID NO 40) | |
| | GACACACAAAACUCUAU, |
| | and |
| | (positions 6951-6968 of SEQ ID NO 45) |
| (SEQ ID NO 41) | |
| | GAGAAAAACCUAUAAUGC. |

In some embodiments, the sequence of the antisense oligonucleotide and/or the sequence of the contiguous nucleotide sequence comprises or consists of a sequence having at least 80% identity to any one of the following sequences:

| (SEQ ID NO 1) | |
|---|---|
| | AGGCATTATAGGTTTTTE, |
| (SEQ ID NO 2) | |
| | AAAAGTTTCTCAGATAGGE, |
| (SEQ ID NO 3) | |
| | GGACATGTTAATTTTACTT, |
| (SEQ ID NO 4) | |
| | TGAACTTGTTATATACETG, |
| (SEQ ID NO 5) | |
| | TAGAGTTTTGTGTGTET, |
| (SEQ ID NO 6) | |
| | GEATTATAGGTTTTTET, |
| (SEQ ID NO 7) | |
| | GGCATTATAGGTTTTTE, |
| (SEQ ID NO 8) | |
| | GGEATTATAGGTTTTTE, |
| (SEQ ID NO 9) | |
| | TGGACAUGTTAATTTTAE, |
| (SEQ ID NO 10) | |
| | GGACATGUTAATTTTACTT, |
| (SEQ ID NO 11) | |
| | GTTTTAAGATTGTGGTGTTT, |
| (SEQ ID NO 12) | |
| | TAGAGTTTUGTGTGTET, |
| (SEQ ID NO 13) | |
| | TAGAGTTTTGTGUGTET, |
| (SEQ ID NO 14) | |
| | ATAGAFTTTTGTGTGTE, |
| (SEQ ID NO 15) | |
| | AAAAFTTTCTCAGATAGGE, |
| (SEQ ID NO 16) | |
| | AAAAGTTTCTCAFATAGGE, |
| (SEQ ID NO 17) | |
| | AAAAGTTUCTCAGATAGGE, |
| (SEQ ID NO 18) | |
| | AGGCATTATAGGUTTTTE, |
| (SEQ ID NO 19) | |
| | AGGCAUTATAGGTTTTTE, |
| (SEQ ID NO 20) | |
| | GEATTATAGGTTTTTCTE, |
| (SEQ ID NO 21) | |
| | GEATTATAGGTTTTUCTE, |
| (SEQ ID NO 22) | |
| | GEATTATAGGTTTUTCTE, |
| (SEQ ID NO 23) | |
| | GEATTATAGGTTUTTCTE, |
| (SEQ ID NO 24) | |
| | GEATTATAGGTTUTTET, |
| (SEQ ID NO 25) | |
| | GEATTATAGGTUTTTET, |
| (SEQ ID NO 26) | |
| | GEATTATAGGUTTTTCTE, |
| (SEQ ID NO 27) | |
| | GEATTATAGGUTTTTET, |
| (SEQ ID NO 28) | |
| | GEATTAUAGGTTTTTET, |
| (SEQ ID NO 29) | |
| | GEATUATAGGTTTTTET, |
| | and |
| (SEQ ID NO 30) | |
| | GEAUTATAGGTTTTTET. |

In some embodiments, the antisense oligonucleotide comprises or consists of any one of CMP ID NOs 1_1, 2_1, 3_1, 4_1, 4_2, 5_1, 5_2, 6_1, 7_1, 7_2, 8_1, 2_2, 9_1, 10_1, 11_1, 4_3, 12_1, 5_3, 13_1, 14_1, 15_1, 16_1, 16_2, 16_3, 2_3, 2_4, 2_5, 2_6, 2_7, 2_8, 2_9, 2_10, 2_11, 2_12, 17_1, 18_1, 19_1, 20_1, 20_2, 20_3, 20_4, 20_5, 20_6, 6_2, 6_3, 6_4, 6_5, 6_6, 6_7, 6_8, 6_9, 6_10, 6_11, 6_12, 21_1, 22_1, 23_1, 24_1, 25_1, 252, 26_1, 27_1, 27_2, 28_1, 29_1, 30_1, 11_2 and 11_3.

The invention also provides an antisense oligonucleotide conjugate comprising the antisense oligonucleotide of the invention covalently attached to at least one conjugate moiety.

In some embodiments, the antisense oligonucleotide conjugate comprises or consists of any one of CNJ ID NOs 1_1, 2_1, 3_1, 4_1, 4_2, 5_1, 5_2, 6_1, 7_1, 7_2, 8_1, 2_2, 9_1, 10_1, 11_1, 4_3, 12_1, 5_3, 13_1, 14_1, 15_1, 16_1, 16_2, 16_3, 2_3, 2_4, 2_5, 2_6, 2_7, 2_8, 2_9, 2_10, 2_11, 2_12, 171, 18_1, 19_1, 20_1, 20_2, 203, 20_4, 20_5, 20_6, 6_2, 6_3, 6_4, 6_5, 6_6, 6_7, 6_8, 6_9, 6_10, 6_11, 6_12, 21_1, 22_1, 23_1, 24_1, 25_1, 25_2, 26_1, 27_1, 27_2, 28_1, 29_1, 30_1, 11_2 and 11_3.

The invention also provides an antisense oligonucleotide conjugate selected from the group consisting of:
(a) CNJ ID NO 1_1 as depicted in FIG. 1;
(b) CNJ ID NO 2_1 as depicted in FIG. 2;
(c) CNJ ID NO 3_1 as depicted in FIG. 3;
(d) CNJ ID NO 4_1 as depicted in FIG. 4;
(e) CNJ ID NO 4_2 as depicted in FIG. 5;
(f) CNJ ID NO 5_1 as depicted in FIG. 6; and
(g) CNJ ID NO 5_2 as depicted in FIG. 7.

In some embodiments of the antisense oligonucleotide or the antisense oligonucleotide conjugate of the invention, the target sequence is SEQ ID NO 32. In some such embodiments, the sequence of the antisense oligonucleotide and/or the sequence of the contiguous nucleotide sequence comprises or consists of a sequence having at least 80% identity to any one of SEQ ID NOs 2, 15, 16 or 17. In some embodiments, the antisense oligonucleotide comprises or consists of any one of CMP ID NOs 2_1, 2_2, 15_1, 16_1, 16_2, 16_3, 2_3, 2_4, 2_5, 2_6, 2_7, 2_8, 2_9, 210, 2_11, 2_12, and 17_1. In some embodiments, the antisense oligonucleotide conjugate comprises or consists of any one of CNJ ID NOs 2_1, 2_2, 15_1, 16_1, 16_2, 16_3, 2_3, 2_4, 2_5, 2_6, 2_7, 2_8, 2_9, 2_10, 2_11, 212, and 17_1.

In some embodiments of the antisense oligonucleotide or the antisense oligonucleotide conjugate of the invention, the target sequence is selected from SEQ ID NOs 31, 36, 37 and 41. In some embodiments, the sequence of the antisense oligonucleotide and/or the sequence of the contiguous nucleotide sequence comprises or consists of a sequence having at least 80% identity to any one SEQ ID NOs 1, 6, 7, 8, 18, 19, 20, 21, 22, 23, 24, 25, 26, 27, 28, 29, or 30. In some embodiments, the antisense oligonucleotide comprises or consists of any one of CMP ID NOs 1_1, 6_1, 7_1, 7_2, 8_1, 181, 101, 201, 202, 203, 204, 205, 206, 6_2, 6_3, 6_4, 6_5, 6_6, 6_7, 6_8, 6_9, 6_10, 6_11, 6_12, 21_1, 22_1, 23_1, 24_1, 25_1, 25_2, 26_1, 27_1, 272, 28_1, 29_1, and 30_1. In some embodiments, the antisense oligonucleotide conjugate comprises or consists of any one of CNJ ID NOs 1_1, 6_1, 7_1, 7_2, 8_1, 18_1, 10_1, 20_1, 20_2, 20_3, 20_4, 20_5, 20_6, 6_2, 6_3, 6_4, 6_5, 6_6, 6_7, 6_8, 6_9, 6_10, 6_11, 6_12, 21_1, 22_1, 23_1, 24_1, 25_1, 25_2, 26_1, 27_1, 27_2, 28_1, 29_1, and 30_1.

In some embodiments of the antisense oligonucleotide or antisense oligonucleotide conjugate of the invention, the target sequence is SEQ ID NO 33 or SEQ ID NO 38. In some embodiments, the sequence of the antisense oligonucleotide and/or the sequence of the contiguous nucleotide sequence comprises or consists of a sequence having at least 80% identity to any one of SEQ ID NOs 3, 9 or 10. In some embodiments, the antisense oligonucleotide comprises or consists of any one of CMP ID NOs 3_1, 9_1, and 10_1. In some embodiments, the antisense oligonucleotide conjugate comprises or consists of any one of CNJ ID NOs 3_1, 9_1, or 10_1.

In some embodiments of the antisense oligonucleotide or the antisense oligonucleotide conjugate of the invention, the target sequence is SEQ ID NO 39. In some embodiments, the sequence of the antisense oligonucleotide and/or the sequence of the contiguous nucleotide sequence comprises or consists of a sequence having at least 80% identity to SEQ ID NO 11.

In some embodiments, the antisense oligonucleotide comprises or consists of any one of CMP ID NOs 11_1, 11_2 or 11_3. In some embodiments, the antisense oligonucleotide conjugate comprises or consists of any one of CNJ ID NOs 11_1, 11_2 or 11_3.

In some embodiments of the antisense oligonucleotide or antisense oligonucleotide conjugate of the invention, the target sequence is SEQ ID NO 34. In some embodiments, the sequence of the antisense oligonucleotide and/or the sequence of the contiguous nucleotide sequence comprises or consists of a sequence having at least 80% identity to SEQ ID NO 4. In some embodiments, the antisense oligonucleotide comprises or consists of any one of CMP ID NOs 4_1, 4_2, or 4_3. In some embodiments, the antisense oligonucleotide conjugate comprises or consists of any one of CNJ ID NOs 4_1, 4_2, or 4_3.

In some embodiments of the antisense oligonucleotide or antisense oligonucleotide conjugate of the invention, the target sequence is SEQ ID NO 35 or SEQ ID NO 40. In some embodiments, the sequence of the antisense oligonucleotide and/or the sequence of the contiguous nucleotide sequence comprises or consists of a sequence having at least 80% identity to any one of SEQ ID NOs 5, 12, 13 or 14. In some embodiments, the antisense oligonucleotide comprises or consists of any one of CMP ID NOs 5_1, 5_2, 12_1, 5_3, 13_1, or 14_1. In some embodiments, the antisense oligonucleotide conjugate comprises or consists of any one of CNJ ID NOs 5_1, 5_2, 12_1, 5_3, 13_1 or 14_1.

The invention also provides a pharmaceutical composition comprising the antisense oligonucleotide or the antisense oligonucleotide conjugate of the invention, and a pharmaceutically acceptable diluent, solvent, carrier, salt and/or adjuvant.

The invention also provides an in vitro or in vivo method for reducing A1CF expression in a target cell, the method comprising administering an effective amount of the antisense oligonucleotide or the antisense oligonucleotide conjugate of the invention or the pharmaceutical composition of the invention to the target cell.

The invention also provides a method for treating or preventing a disease comprising administering a therapeutically or prophylactically effective amount of the antisense oligonucleotide or the antisense oligonucleotide conjugate of the invention or the pharmaceutical composition of the invention to a subject suffering from or susceptible to a disease.

The invention also provides the antisense oligonucleotide or the antisense oligonucleotide conjugate of the invention or the pharmaceutical composition of the invention, for use in the treatment or prevention of a disease in a subject.

The invention also provides use of the antisense oligonucleotide or the antisense oligonucleotide conjugate of the invention or the pharmaceutical composition of the invention, for the preparation of a medicament for treatment or prevention of a disease in a subject.

In some embodiments, the disease is HBV infection. In preferred embodiments, the disease is chronic HBV infection.

### BRIEF DESCRIPTION OF FIGURES

FIG. 1 shows the chemical structure of CNJ ID NO 1_1 (corresponding to SEQ ID NO 1 and CMP ID NO 1_1).

FIG. 2 shows the chemical structure of CNJ ID NO 2_1 (corresponding to SEQ ID NO 2 and CMP ID NO 2_1).

FIG. 3 shows the chemical structure of CNJ ID NO 3_1 (corresponding to SEQ ID NO 3 and CMP ID NO 3_1).

FIG. 4 shows the chemical structure of CNJ ID NO 4_1 (corresponding to SEQ ID NO 4 and CMP ID NO 4_1).

FIG. 5 shows the chemical structure of CNJ ID NO 4_2 (corresponding to SEQ ID NO 4 and CMP ID NO 4_2).

FIG. 6 shows the chemical structure of CNJ ID NO 5_1 (corresponding to SEQ ID NO 5 and CMP ID NO 5_1).

FIG. 7 shows the chemical structure of CNJ ID NO 5_2 (corresponding to SEQ ID NO 5 and CMP ID NO 5_2).

FIG. 8 shows the chemical structure of aminohexyl conjugated tri(N-acetyl-galactosamine), (HELM annotation [5gn2c6]), which is the GalNAc moiety in each of antisense oligonucleotide conjugate presented in Table 5. The wavy line indicates the covalent bond by which the GalNAc moiety is attached to the linker or to the antisense oligonucleotide.

The chemical drawings show the protonated form of the antisense oligonucleotide, and it will be understood that each hydrogen on the sulphur atom in the phosphorothioate internucleoside linkage may independently be present or absent. It will be understood that the presence of the proton will depend on the acidity of the environment of the molecule. In a salt form, one or more of the hydrogens may for example be replaced with a cation, such as a metal cation, such as a sodium cation or a potassium cation. Protonated phosphorothioates exist in tautomeric forms.

### DETAILED DESCRIPTION OF THE INVENTION

Unless otherwise stated, all ranges are inclusive of the start and end value.

Each reference herein to a SEQ ID NO (sequence identifier number) refers to the sequence represented by that SEQ ID NO. Each reference herein to CMP ID NO (compound identifier number) refers to the compound represented by that CMP ID NO. Each reference herein to CNJ ID NO (conjugate identifier number) refers to the conjugate represented by that CNJ ID NO.

The invention provides an antisense oligonucleotide of 12 to 30 nucleotides in length, which comprises a contiguous nucleotide sequence of 12 to 30 nucleotides in length, wherein the contiguous nucleotide sequence is capable of binding to a target sequence in an apolipoprotein B mRNA editing enzyme catalytic subunit 1 (APOBEC1) Complementation Factor (A1CF) mRNA, wherein the target sequence is a sequence of at least 17 contiguous nucleotides within any of the following sequences:

| | (positions 2181-2199 of SEQ ID NO 45) |
|---|---|
| (SEQ ID NO 32) | |
| | GCCUAUCUGAGAAACUUUU, |
| | (positions 6951-6970 of SEQ ID NO 45) |
| (SEQ ID NO 42) | |
| | GAGAAAAACCUAUAAUGCCU, |
| | (positions 16970-16989 of SEQ ID NO 45) |
| (SEQ ID NO 43) | |
| | AAGUAAAAUUAACAUGUCCA, |
| | (positions 26358-26377 of SEQ ID NO 45) |
| (SEQ ID NO 39) | |
| | AAACACCACAAUCUUAAAAC, |
| | (positions 38053-38071 of SEQ ID NO 45) |
| (SEQ ID NO 34) | |
| | CAGGUAUAUAACAAGUUCA, |
| | and |
| | (positions 78973-78990 of SEQ ID NO 45) |
| (SEQ ID NO 44) | |
| | AGACACACAAAACUCUAU, |

and wherein the antisense oligonucleotide is capable of reducing A1CF expression.

Oligonucleotide

The term “oligonucleotide” as used herein is defined, as is generally understood by the skilled person, as a molecule comprising two or more covalently linked nucleosides. Such covalently bound nucleosides may also be referred to as nucleic acid molecules or oligomers.

Oligonucleotides are commonly made in a laboratory by solid-phase chemical synthesis followed by purification and isolation. When referring to the sequence of an oligonucleotide, reference is made to the sequence or order of nucleobase moieties, or modifications thereof, of the covalently linked nucleotides or nucleosides. The oligonucleotides of the invention are man-made, and are chemically synthesized, and are typically purified or isolated.

Nucleotides and Nucleosides

Nucleotides and nucleosides are the building blocks of oligonucleotides and polynucleotides, and for the purposes of the present invention include both naturally occurring and non-naturally occurring nucleotides and nucleosides. In nature, nucleotides, such as DNA and RNA nucleotides, comprise a ribose sugar moiety, a nucleobase moiety and one or more phosphate groups. The one or more phosphate groups are absent in nucleosides. Nucleosides and nucleotides may also interchangeably be referred to as “units” or “monomers”.

Nucleobase

The term nucleobase includes the purine (e.g. adenine and guanine) and pyrimidine (e.g. uracil, thymine and cytosine) moiety present in nucleosides and nucleotides which form hydrogen bonds in nucleic acid hybridization. In the context of the present invention the term nucleobase also encompasses modified nucleobases which may differ from naturally occurring nucleobases, but which are functional during nucleic acid hybridization. In this context “nucleobase” refers to both naturally occurring nucleobases such as adenine, guanine, cytosine, thymidine, uracil, xanthine and hypoxanthine, as well as non-naturally occurring variants. Such variants are for example described in Hirao et al., 2012, Accounts of Chemical Research, 45, 2055-2065 and Bergstrom, 2009, Curr. Protoc. Nucleic Acid Chem., 37, 1.4.1-1.4.32.

In some embodiments the nucleobase moiety is modified by changing the purine or pyrimidine into a modified purine or pyrimidine, such as substituted purine or substituted pyrimidine, such as a nucleobase selected from isocytosine, pseudoisocytosine, 5-methyl cytosine, 5-thiozolo-cytosine, 5-propynyl-cytosine, 7-deaza-8-azaguanine, 5-propynyl-uracil, 5-bromouracil 5-thiazolo-uracil, 2-thio-uracil, 2′thio-thymine, inosine, diaminopurine, 6-aminopurine, 2-aminopurine, 2,6-diaminopurine and 2-chloro-6-aminopurine.

The nucleobase moieties may be indicated by the letter code for each corresponding nucleobase, e.g. A, T, G, C or U, wherein each letter may optionally include modified nucleobases of equivalent function. For example, in the exemplified oligonucleotides, the nucleobase moieties are selected from A, T, G, C, 5-methyl cytosine and 7-deaza-8-azaguanine. Optionally, for LNA gapmers, 5-methyl cytosine LNA nucleosides may be used. 5-methyl cytosine may be denoted as “E”. 7-deaza-8-azaguanine may be denoted as “F”.

Antisense Oligonucleotide

The oligonucleotide is an antisense oligonucleotide.

The term “antisense oligonucleotide” as used herein is defined as an oligonucleotide capable of modulating expression of a target gene by hybridizing to a target nucleic acid, in particular to a contiguous sequence on a target nucleic acid. Antisense oligonucleotides are not essentially double stranded and are therefore not siRNAs or shRNAs. In some embodiments, the antisense oligonucleotide is a single stranded antisense oligonucleotide. It is understood that single stranded oligonucleotides of the present invention can form hairpins or intermolecular duplex structures (duplex between two molecules of the same oligonucleotide), as long as the degree of intra or inter self-complementarity is less than approximately 50% across of the full length of the oligonucleotide.

In some embodiments, the antisense oligonucleotides of the invention do not contain RNA nucleosides.

The term “antisense oligonucleotide” may be abbreviated as “ASO” herein.

Length of the Antisense Oligonucleotide

The antisense oligonucleotide of the invention is 12 to 30 nucleotides in length. In some embodiments, the antisense oligonucleotide is 12, 13, 14, 15, 16, 17, 18, 19, 20, 21, 22, 23, 24, 25, 26, 27, 28, 29 or 30 nucleotides in length. In some embodiments, the antisense oligonucleotide is 15 to 25 nucleotides in length, such as 15 to 24, 23, 22, 21 or 20 nucleotides in length. In some embodiments, the antisense oligonucleotide is 16 to 25 nucleotides in length, such as 16 to 24, 23, 22, 21 or 20 nucleotides in length. In some embodiments, the antisense oligonucleotide is 17 to 25 nucleotides in length, such as 17 to 24, 23, 22, 21 or 20 nucleotides in length. In some embodiments, the antisense oligonucleotide is 17, 18, 19 or 20 nucleotides in length. In some embodiments, the antisense oligonucleotide is 17 nucleotides in length. In some embodiments, the antisense oligonucleotide is 18 nucleotides in length. In some embodiments, the antisense oligonucleotide is 19 nucleotides in length. In some embodiments, the antisense oligonucleotide is 20 nucleotides in length.

Contiguous Nucleotide Sequence

The term “contiguous nucleotide sequence” refers to the region of the antisense oligonucleotide which binds to A1CF mRNA. In other words, it is the contiguous nucleotide sequence which mediates binding of the antisense oligonucleotide of the invention to A1CF mRNA. The contiguous nucleotide sequence mediates binding of the antisense oligonucleotide of the invention to the target sequence in A1CF mRNA. Accordingly, the contiguous nucleotide sequence is complementary to, and in some instances fully complementary to, the target sequence. The term “contiguous nucleotide sequence” is used interchangeably herein with the term “contiguous nucleobase sequence”.

In some embodiments, the antisense oligonucleotide comprises the contiguous nucleotide sequence, and may optionally comprise further nucleotide(s), for example a nucleotide linker sequence which may be used to attach a functional group (e.g. a conjugate group) to the contiguous nucleotide sequence. The nucleotide linker sequence may or may not be complementary to the target nucleic acid.

Length of the Contiguous Nucleotide Sequence

It is understood that the contiguous nucleotide sequence of the antisense oligonucleotide cannot be longer than the antisense oligonucleotide as such and that the antisense oligonucleotide cannot be shorter than the contiguous nucleotide sequence.

The contiguous nucleotide sequence of the antisense oligonucleotide of the invention is 12 to 30 nucleotides in length. In some embodiments, the contiguous nucleotide sequence is 12, 13, 14, 15, 16, 17, 18, 19, 20, 21, 22, 23, 24, 25, 26, 27, 28, 29 or 30 nucleotides in length. In some embodiments, the contiguous nucleotide sequence is 15 to 25 nucleotides in length, such as 15 to 24, 23, 22, 21 or 20 nucleotides in length. In some embodiments, contiguous nucleotide sequence is 16 to 25 nucleotides in length, such as 16 to 24, 23, 22, 21 or 20 nucleotides in length. In some embodiments, the contiguous nucleotide sequence is 17 to 25 nucleotides in length, such as 17 to 24, 23, 22, 21 or 20 nucleotides in length. In some embodiments, the contiguous nucleotide sequence is 17, 18, 19 or 20 nucleotides in length. In some embodiments, the contiguous nucleotide sequence is 17 nucleotides in length. In some embodiments, the contiguous nucleotide sequence is 18 nucleotides in length. In some embodiments, the contiguous nucleotide sequence is 19 nucleotides in length. In some embodiments, the contiguous nucleotide sequence is 20 nucleotides in length.

In some embodiments, the antisense oligonucleotide consists of the contiguous nucleotide sequence. In some embodiments, the contiguous nucleotide sequence is the same length as the antisense oligonucleotide. In some embodiments, the antisense oligonucleotide is the contiguous nucleotide sequence. In some embodiments, all of the nucleosides of the antisense oligonucleotide constitute the contiguous nucleotide sequence. In some embodiments, the terms “antisense oligonucleotide” and “contiguous nucleotide sequence” are interchangeable.

A1CF mRNA

The contiguous nucleotide sequence of the antisense oligonucleotide of the invention is capable of binding to a target sequence in an A1CF mRNA.

The term “A1CF mRNA” refers to an RNA molecule transcribed from the A1CF gene. In other words, the A1CF mRNA is the mRNA encoded by the A1CF gene. The human A1CF gene is located at positions 50,799,409 to 50,885,675 on the reverse strand of chromosome 10 (genome build GRCh38.p13, Ensembl ID ENSG00000148584). In some embodiments, the A1CF gene may comprise the human A1CF genomic sequence according to NCBI Reference Sequence NG_029916.1. The sense strand of the DNA sequence of the human A1CF gene is presented herein as SEQ ID NO 45.

The A1CF gene is transcribed to pre-mRNA. Thus, in some embodiments, the A1CF mRNA is human A1CF pre-mRNA. The sequence of A1CF pre-mRNA is considered herein to correspond to SEQ ID NO 45 (the DNA sequence of the sense strand of the human A1CF gene). As is well known in the art, each thymine nucleobase in a DNA sequence is, in the corresponding RNA sequence, replaced with a uracil nucleobase. Thus the A1CF pre-mRNA sequence corresponds to SEQ ID NO 45 wherein each thymine nucleobase is replaced with a uracil nucleobase. References to SEQ ID NO 45 herein accordingly may refer to the gene DNA sequence (comprising thymines) or the pre-mRNA RNA sequence (wherein each thymine is replaced with a uracil). The positions in the tables herein refer to positions in the human A1CF gene/pre-mRNA (i.e. SEQ ID NO 45). Any reference to the A1CF gene sequence also encompasses reference to the A1CF pre-mRNA sequence corresponding to the A1CF gene sequence. Accordingly, the A1CF mRNA may be an mRNA sequence which is encoded by SEQ ID NO 45.

Human A1CF pre-mRNA is alternatively spliced to any of 10 variant transcripts, 8 of which comprise an open reading frame that may be translated to protein. These spliced mRNAs may be referred to herein as “mature mRNAs”. Thus, in some embodiments, the A1CF mRNA is a human A1CF mature mRNA. The NCBI Reference Sequences in the NCBI database (ncbi.nlm.nih.gov) of the human A1CF gene/pre-mRNA and the 8 protein coding mature mRNA variants are presented with their corresponding SEQ ID NOs in Table 1 below.

TABLE 1
Human A1CF gene and mRNA sequences

| | | SEQ |
|---|---|---|
| Sequence | NCBI Ref Seq | ID NO |
| Human A1CF gene/pre-mRNA | NG_029916.1 | 45 |
| Human A1CF-201 (transcript variant 1) | NM_014576.4 | 46 |
| Human A1CF-202 (transcript variant 2) | NM_138932.3 | 47 |
| Human A1CF-203 (transcript variant 3) | NM_138933.3 | 48 |
| Human A1CF-204 (transcript variant 4) | NM_001198818.2 | 49 |
| Human A1CF-205 (transcript variant 5) | NM_001198819.2 | 50 |
| Human A1CF-206 (transcript variant 6) | NM_001198820.2 | 51 |
| Human A1CF-205 (transcript variant 7) | NM_001370130.1 | 52 |
| Human A1CF-205 (transcript variant 8) | NM_001370131.1 | 53 |

Thus, in some embodiments, the A1CF mRNA comprises a sequence selected from the group consisting of SEQ ID NOs 45, 46, 47, 48, 49, 50, 51, 52 and 53. In some embodiments, the A1CF mRNA consists of a sequence selected from the group consisting of SEQ ID NOs 45, 46, 47, 48, 49, 50, 51, 52 and 53. In some embodiments, the A1CF mRNA comprises SEQ ID NO 45. In some embodiments, the A1CF mRNA consists of SEQ ID NO 45.

SEQ ID NO 45 is provided herein as a reference sequence and it will be understood that the target nucleic acid may be a variant of SEQ ID NO 45, such as a naturally occurring variant, such as an allelic variant, which comprises one or more polymorphisms in the human A1CF sequence. The term “naturally occurring variant” refers to variants of the A1CF gene or transcripts which originate from the same genetic loci as the A1CF mRNA, but may differ for example, by virtue of degeneracy of the genetic code causing a multiplicity of codons encoding the same amino acid, or due to alternative splicing of pre-mRNA, or the presence of polymorphisms, such as single nucleotide polymorphisms (SNPs), and allelic variants. Based on the presence of the sufficient complementary sequence to the oligonucleotide, the oligonucleotide of the invention may therefore target A1CF mRNA and naturally occurring variants thereof.

Accordingly, in some embodiments, the A1CF mRNA comprises or consists of a sequence having at least 75%, at least 80%, at least 85%, at least 90%, at least 95%, at least 96%, at least 97%, at least 98%, at least 99%, or 100% (i.e. full) sequence identity to a sequence selected from the group consisting of SEQ ID NOs 45, 46, 47, 48, 49, 50, 51, 52 and 53. In some embodiments, the A1CF mRNA comprises or consists of a sequence having at least 75%, at least 80%, at least 85%, at least 90%, at least 95%, at least 96%, at least 97%, at least 98%, at least 99%, or 100% (i.e. full) sequence identity to the sequence according to SEQ ID NO 45.

Binding to A1CF mRNA

The contiguous nucleotide sequence of the antisense oligonucleotide of the invention is capable of binding to a target sequence in an A1CF mRNA.

The terms “is capable of binding”, “binds” and “can bind” are interchangeable herein. The term “is capable of binding” means that the contiguous nucleotide sequence can associate with the target sequence in an A1CF mRNA. This is achieved by complementary base pairing, as described herein. The contiguous nucleotide sequence need not bind to the entirety of the target sequence. Likewise, not all of the contiguous nucleotide sequence need bind to the target sequence. It is sufficient that at least part of the contiguous nucleotide sequence is capable of binding to at least part of the target sequence, thereby associating the antisense oligonucleotide with the A1CF mRNA. Thus, in some embodiments, at least part of the contiguous nucleotide sequence is capable of binding to at least part of the target sequence.

The contiguous nucleotide sequence thus mediates binding of the antisense oligonucleotide to the A1CF mRNA. Accordingly, in some embodiments the antisense oligonucleotide is capable of binding to a target sequence in an A1CF mRNA. In some embodiments the antisense oligonucleotide conjugate is capable of binding to a target sequence in an A1CF mRNA.

Target Sequence

The contiguous nucleotide sequence of the antisense oligonucleotide of the invention is capable of binding to a target sequence in an A1CF mRNA.

The term “target sequence” refers to the region of A1CF mRNA to which the contiguous nucleotide sequence or antisense oligonucleotide binds. The term “target sequence” is interchangeable with the terms “target site”, “target nucleic acid”, “target site sequence”, “target nucleic acid sequence” and “target site nucleic acid sequence”.

The target sequences to which the contiguous nucleotide sequence or antisense oligonucleotide of the invention is capable of binding are located in six regions in the A1CF mRNA sequence. These regions are at positions 2181-2199, 6951-6970, 16970-16989, 26358-26377, 38053-38071 and 78973-78990 of the A1CF gene/pre-mRNA sequence (SEQ ID NO 45). In some embodiments, these regions are the target sequences to which the contiguous nucleotide sequence or antisense oligonucleotide of the invention is capable of binding.

In some embodiments, the contiguous nucleotide sequences of the antisense oligonucleotides of the invention are capable of binding to shorter target sequences within these regions. In particular, within the target sequence at positions 6951-6970 are at least four target sequences at positions 6951-6968, 6952-6968, 6953-6969 and 6953-6970; within the target sequence at positions 16970-16988 are at least two target sequences at positions 16970-16988 and 16972-16989; and within the target sequence at positions 78973-78990 are at least two target sequences at positions 78973-78989 and 78974-78990. All of these target sequences are set out below in Table 2.

TABLE 2
Target sequences

| | | Position in |
|---|---|---|
| Target | | A1CF gene |
| SEQ ID | Target nucleobase | NG029916.1 |
| NO | sequence | (SEQ ID NO 45) |
| 32 | GCCUAUCUGAGAAACUUUU | 2181-2199 |
| 41 | GAGAAAAACCUAUAAUGC | 6951-6968 |
| 36 | AGAAAAACCUAUAAUGC | 6952-6968 |
| 37 | GAAAAACCUAUAAUGCC | 6953-6969 |
| 31 | GAAAAACCUAUAAUGCCU | 6953-6970 |
| 42 | GAGAAAAACCUAUAAUGCCU | 6951-6970 |
| 33 | AAGUAAAAUUAACAUGUCC | 16970-16988 |
| 38 | GUAAAAUUAACAUGUCCA | 16972-16989 |
| 43 | AAGUAAAAUUAACAUGUCCA | 16970-16989 |
| 39 | AAACACCACAAUCUUAAAAC | 26358-26377 |
| 34 | CAGGUAUAUAACAAGUUCA | 38053-38071 |
| 35 | AGACACACAAAACUCUA | 78973-78989 |
| 40 | GACACACAAAACUCUAU | 78974-78990 |
| 44 | AGACACACAAAACUCUAU | 78973-78990 |

Code for nucleobases:
A = adenine,
C = cytosine,
E = 5-methyl cytosine,
F = 7-deaza-8-azaguanine,
G = guanine,
T = thymine,
U = uracil

Thus, the target sequence for the antisense oligonucleotide of the invention is a sequence of at least 17 contiguous nucleotides within any of the following sequences:

| | (positions 2181-2199 of SEQ ID NO 45) |
|---|---|
| (SEQ ID NO 32) | |
| | GCCUAUCUGAGAAACUUUU, |
| | (positions 6951-6970 of SEQ ID NO 45) |
| (SEQ ID NO 42) | |
| | GAGAAAAACCUAUAAUGCCU, |
| | (positions 16970-16989 of SEQ ID NO 45) |
| (SEQ ID NO 43) | |
| | AAGUAAAAUUAACAUGUCCA, |
| | (positions 26358-26377 of SEQ ID NO 45) |
| (SEQ ID NO 39) | |
| | AAACACCACAAUCUUAAAAC, |
| | (positions 38053-38071 of SEQ ID NO 45) |
| (SEQ ID NO 34) | |
| | CAGGUAUAUAACAAGUUCA, |
| | and |
| | (positions 78973-78990 of SEQ ID NO 45) |
| (SEQ ID NO 44) | |
| | AGACACACAAAACUCUAU. |

In some embodiments, the target sequence is a sequence of at least 18, 19 or 20 contiguous nucleotides within any of SEQ ID NOs 32, 42, 43, 39, 34 or 44. In some embodiments, the target sequence is a sequence of 17, 18, 19 or 20 contiguous nucleotides within any of SEQ ID NOs 32, 42, 43, 39, 34 or 44.

In some embodiments, the target sequence is selected from the following sequences:

| | (positions 6953-6970 of SEQ ID NO 45) |
|---|---|
| (SEQ ID NO 31) | |
| | GAAAAACCUAUAAUGCCU, |
| | (positions 2181-2199 of SEQ ID NO 45) |
| (SEQ ID NO 32) | |
| | GCCUAUCUGAGAAACUUUU, |
| | (positions 16970-16988 of SEQ ID NO 45) |
| (SEQ ID NO 33) | |
| | AAGUAAAAUUAACAUGUCC, |
| | (positions 38053-38071 of SEQ ID NO 45) |
| (SEQ ID NO 34) | |
| | CAGGUAUAUAACAAGUUCA, |
| | (positions 78973-78989 of SEQ ID NO 45) |
| (SEQ ID NO 35) | |
| | AGACACACAAAACUCUA, |
| | (positions 6952-6968 of SEQ ID NO 45) |
| (SEQ ID NO 36) | |
| | AGAAAAACCUAUAAUGC, |
| | (positions 6953-6969 of SEQ ID NO 45) |
| (SEQ ID NO 37) | |
| | GAAAAACCUAUAAUGCC, |
| | (positions 16972-16989 of SEQ ID NO 45) |
| (SEQ ID NO 38) | |
| | GUAAAAUUAACAUGUCCA, |
| | (positions 26358-26377 of SEQ ID NO 45) |
| (SEQ ID NO 39) | |
| | AAACACCACAAUCUUAAAAC, |
| | (positions 78974-78990 of SEQ ID NO 45) |
| (SEQ ID NO 40) | |
| | GACACACAAAACUCUAU, |
| | and |
| | (positions 6951-6968 of SEQ ID NO 45) |
| (SEQ ID NO 41) | |
| | GAGAAAAACCUAUAAUGC. |

In some embodiments, the target sequence is selected from SEQ ID NOs 31, 32, 33, 34, 35, 36, 37, 38, 39 and 40. In some embodiments, the target sequence is selected from SEQ ID NOs 31, 32, 33, 34 and 35.

In some embodiments, the target sequence is SEQ ID NO 31. In some embodiments, the target sequence is SEQ ID NO 32. In some embodiments, the target sequence is SEQ ID NO 33. In some embodiments, the target sequence is SEQ ID NO 34. In some embodiments, the target sequence is SEQ ID NO 35. In some embodiments, the target sequence is SEQ ID NO 36. In some embodiments, the target sequence is SEQ ID NO 37. In some embodiments, the target sequence is SEQ ID NO 38. In some embodiments, the target sequence is SEQ ID NO 39. In some embodiments, the target sequence is SEQ ID NO 40. In some embodiments, the target sequence is SEQ ID NO 41.

Reducing A1CF Expression

The antisense oligonucleotide of the invention is capable of reducing A1CF expression.

The term “A1CF expression” refers to how much gene product is produced from the A1CF gene. In some embodiments, A1CF expression is the amount of A1CF mRNA produced from the A1CF gene (i.e. the amount of A1CF mRNA expressed). The amount of A1CF mRNA expressed may be determined by techniques known in the art, such as quantitative PCR or Northern blotting. In some embodiments, A1CF expression is the amount of A1CF protein produced from the A1CF gene (i.e. the amount of A1CF protein expressed). The amount of A1CF protein expressed may be determined by techniques known in the art, such as Western blotting.

The term “reduction of expression” as used herein is to be understood as an overall term for an oligonucleotide's ability to inhibit the amount or the activity of A1CF in a target cell. Reduction of activity may be determined by measuring the level of A1CF pre-mRNA or A1CF mRNA. Reduction of expression may therefore be determined in vitro or in vivo.

The term “reduction” or “reduce” may also be referred as down-regulate, inhibit, suppress, lessen, lower, the expression of A1CF, such as A1CF pre-mRNA.

The reduction of expression may occur by degradation of pre-mRNA or mRNA (e.g. using RNase H recruiting oligonucleotides, such as gapmers).

The effect of the antisense oligonucleotide on A1CF expression may be assessed by exposing a cell to an antisense oligonucleotide of the invention, such as by transfecting the cell with an antisense oligonucleotide of the invention, and then determining A1CF expression levels. Thus, in some embodiments, the antisense oligonucleotide is capable of reducing A1CF expression in a cell. In some embodiments, the antisense oligonucleotide is capable of reducing A1CF expression in a cell which expresses A1CF. In some embodiments, A1CF expression is the amount of A1CF mRNA expressed in the cell. In some embodiments, A1CF expression is the amount of A1CF protein expressed in the cell.

In some embodiments, the antisense oligonucleotide is capable of reducing A1CF expression by at least 10%, at least 20%, at least 30%, at least 40%, at least 50%, at least 60%, at least 70%, at least 75%, at least 80%, at least 85%, at least 90%, at least 95% or 100% (i.e. completely abolishing A1CF expression).

A person skilled in the art is aware that quantification of any biological parameter requires comparison to a control. Typically, reduction of expression is determined by comparing the reduction of activity due to the administration of an effective amount of the antisense oligonucleotide to the target cell and comparing that level to a reference level obtained from a target cell without administration of the antisense oligonucleotide (control experiment), or a known reference level (e.g. the level of expression prior to administration of the effective amount of the antisense oligonucleotide, or a predetermine or otherwise known expression level). For example a control experiment may be an animal or person, or a target cell treated with a saline composition or a reference oligonucleotide (often a scrambled control). In some embodiments, the control is a cell that has not been exposed to the antisense oligonucleotide.

In some embodiments, the antisense oligonucleotide is capable of reducing A1CF expression compared to a control. In some embodiments, the antisense oligonucleotide is capable of reducing A1CF expression compared to a control by at least 10%, at least 20%, at least 30%, at least 40%, at least 50%, at least 60%, at least 70%, at least 75%, at least 80%, at least 85%, at least 90%, at least 95% or 100% (i.e. completely abolishing A1CF expression).

Reducing HBV Nucleic Acids

The antisense oligonucleotides of the invention are useful in the treatment or prevention of HBV infection, as described herein. Accordingly, in some embodiments the cell is infected with HBV. In some embodiments, the cell is a hepatocyte.

A cell infected with HBV may comprise or be associated with various HBV nucleic acids (i.e. nucleic acids present in the cell due to the HBV infection). Such HBV nucleic acids include intracellular HBV DNA, secreted HBV DNA, pregenomic RNA (pgRNA) and covalently closed circular DNA (cccDNA).

The antisense oligonucleotides and antisense oligonucleotide conjugates of the invention may reduce any or all of these types of HBV nucleic acid. In other words, treating a cell infected with HBV with an antisense oligonucleotide or antisense oligonucleotide conjugate of the invention may reduce any or all of these types of HBV nucleic acid.

The level (and thus the degree of reduction) of these types of HBV nucleic acid may be determined by techniques known in the art and described herein, such as quantitative PCR. It will be understood that the reduction of these types of HBV nucleic acid is determined in comparison to a control. In some embodiments, the control is a cell that has not been exposed to the antisense oligonucleotide.

Reducing HBV DNA

A cell infected with HBV may comprise intracellular HBV DNA, which is the HBV genome. The intracellular HBV DNA may be comprised in live virus, which may be replicating in the cell. The intracellular HBV DNA may be comprised in dead virus.

A cell infected with HBV may secrete HBV DNA into the environment. In the case of a cultured cell, the environment may a culture medium. In the case of a cell in a living organism, the environment may be the extracellular milieu, such as serum. The secreted HBV DNA may be comprised in live virus. The secreted HBV DNA may be comprised in dead virus.

Thus, in some embodiments, the antisense oligonucleotide is capable of reducing total intracellular HBV DNA in the cell. In some embodiments, the antisense oligonucleotide is capable of reducing total intracellular HBV DNA in the cell by at least 10%, at least 20%, at least 30%, at least 40%, at least 50%, at least 60%, at least 70%, at least 75%, at least 80%, at least 85%, at least 90%, at least 95% or 100%.

In some embodiments, the antisense oligonucleotide is capable of reducing total intracellular HBV DNA in the cell compared to a control. In some embodiments, the antisense oligonucleotide is capable of reducing total intracellular HBV DNA in the cell compared to a control by at least 10%, at least 20%, at least 30%, at least 40%, at least 50%, at least 60%, at least 70%, at least 75%, at least 80%, at least 85%, at least 90%, at least 95% or 100%.

In some embodiments, the antisense oligonucleotide is capable of reducing the amount of HBV DNA secreted by the cell. In some embodiments, the antisense oligonucleotide is capable of reducing the amount of HBV DNA secreted by the cell by at least 10%, at least 20%, at least 30%, at least 40%, at least 50%, at least 60%, at least 70%, at least 75%, at least 80%, at least 85%, at least 90%, at least 95% or 100%.

In some embodiments, the antisense oligonucleotide is capable of reducing the amount of HBV DNA secreted by the cell compared to a control. In some embodiments, the antisense oligonucleotide is capable of reducing the amount of HBV DNA secreted by the cell compared to a control by at least 10%, at least 20%, at least 30%, at least 40%, at least 50%, at least 60%, at least 70%, at least 75%, at least 80%, at least 85%, at least 90%, at least 95% or 100%.

Reducing cccDNA

Covalently closed circular DNA (cccDNA) is an episomal form of the HBV genome which is stably maintained in the nucleus. After HBV infects a cell, its circular DNA genome is transferred into the nucleus and repaired to cccDNA by DNA polymerase. The cccDNA serves as a template for all HBV transcripts. cccDNA is needed for productive infection and is responsible for viral persistence during natural course of chronic HBV infection (Locarnini & Zoulim, 2010 Antivir Ther. 15 Suppl 3:3-14. doi: 10.3851/IMP1619). Acting as a viral reservoir, cccDNA is the source of viral rebound after cessation of treatment, necessitating long term, often, lifetime treatment. Consequently, novel therapies that can deliver a complete cure, defined by degradation or elimination of HBV cccDNA, to the majority of CHB patients are highly needed.

In some embodiments, the antisense oligonucleotide is capable of reducing the amount of cccDNA in the cell. In some embodiments, the antisense oligonucleotide is capable of reducing the amount of cccDNA in the cell by at least 10%, at least 20%, at least 30%, at least 40%, at least 50%, at least 60%, at least 70%, at least 75%, at least 80%, at least 85%, at least 90%, at least 95% or 100%.

In some embodiments, the antisense oligonucleotide is capable of reducing the amount of cccDNA in the cell compared to a control. In some embodiments, the antisense oligonucleotide is capable of reducing the amount of cccDNA in the cell compared to a control by at least 10%, at least 20%, at least 30%, at least 40%, at least 50%, at least 60%, at least 70%, at least 75%, at least 80%, at least 85%, at least 90%, at least 95% or 100%.

Reducing pgRNA

Pregenomic RNA (pgRNA) is an RNA molecule transcribed from cccDNA that serves as the template for reverse transcription of HBV genomic DNA. pgRNA is therefore the key intermediate in HBV replication.

In some embodiments, the antisense oligonucleotide is capable of reducing the amount of pgRNA in the cell. In some embodiments, the antisense oligonucleotide is capable of reducing the amount of pgRNA in the cell by at least 10%, at least 20%, at least 30%, at least 40%, at least 50%, at least 60%, at least 70%, at least 75%, at least 80%, at least 85%, at least 90%, at least 95% or 100%.

In some embodiments, the antisense oligonucleotide is capable of reducing the amount of pgRNA in the cell compared to a control. In some embodiments, the antisense oligonucleotide is capable of reducing the amount of pgRNA in the cell compared to a control by at least 10%, at least 20%, at least 30%, at least 40%, at least 50%, at least 60%, at least 70%, at least 75%, at least 80%, at least 85%, at least 90%, at least 95% or 100%.

RNase H Activity and Recruitment

Ribonuclease H (RNase H) refers to a family of endonucleases which cleave the RNA component of RNA/DNA duplexes. RNase H recruitment by antisense oligonucleotides to RNA target sequences followed by RNase H mediated degradation of the RNA target is considered to be one mechanism by which antisense oligonucleotides mediate suppression of RNA targets.

The RNase H activity of an antisense oligonucleotide refers to its ability to recruit RNase H when in a duplex with a complementary RNA molecule. WO01/23613 provides in vitro methods for determining RNaseH activity, which may be used to determine the ability to recruit RNaseH. Typically an oligonucleotide is deemed capable of recruiting RNase H if it, when provided with a complementary target nucleic acid sequence, has an initial rate, as measured in pmol/l/min, of at least 5%, such as at least 10%, at least 20% or more than 20%, of the initial rate determined when using an oligonucleotide having the same base sequence as the modified oligonucleotide being tested, but containing only DNA monomers with phosphorothioate linkages between all monomers in the oligonucleotide, and using the methodology provided by Examples 91-95 of WO01/23613 (hereby incorporated by reference). For use in determining RHase H activity, recombinant RNase H1 is available from Lubio Science GmbH, Lucerne, Switzerland.

DNA oligonucleotides are known to effectively recruit RNaseH, as are gapmer oligonucleotides which comprise a region of DNA nucleosides (typically at least 5 or 6 contiguous DNA nucleosides), flanked 5′ and 3′ by regions comprising 2′ sugar modified nucleosides, typically high affinity 2′ sugar modified nucleosides, such as 2′-O-MOE and/or LNA. In some embodiments, the antisense oligonucleotide of the invention is a gapmer.

For effective facilitation of steric blocking, degradation of the pre-mRNA is not desirable, and as such it is preferable to avoid the RNaseH degradation of the target. Therefore, in some embodiments the antisense oligonucleotides of the invention are not RNaseH recruiting gapmer oligonucleotides. RNaseH recruitment may be avoided by limiting the number of contiguous DNA nucleotides in the oligonucleotide. Therefore mixmer and totalmer designs may be used.

In some embodiments, the antisense oligonucleotide is capable of recruiting RNase H. In some embodiments, the antisense oligonucleotide has RNase H activity. In other words, when the antisense oligonucleotide binds to (i.e. hybridizes with) at least part of the target sequence on the A1CF mRNA, RNAse H is recruited, resulting in cleavage of the A1CF mRNA by RNase H and thereby reduction of A1CF expression.

Nuclease mediated degradation refers to an oligonucleotide capable of mediating degradation of a complementary nucleotide sequence when forming a duplex with such a sequence.

In some embodiments, the oligonucleotide may function via nuclease mediated degradation of the target nucleic acid, where the oligonucleotides of the invention are capable of recruiting a nuclease, particularly an endonuclease, preferably endoribonuclease (RNase), such as RNase H. Examples of oligonucleotide designs which operate via nuclease mediated mechanisms are oligonucleotides which typically comprise a region of at least 5 or 6 consecutive DNA nucleosides and are flanked on one side or both sides by affinity enhancing nucleosides, for example gapmers.

Complementarity

In some embodiments, the contiguous nucleotide sequence of the antisense oligonucleotide of the invention is complementary to a target sequence in an A1CF mRNA. In some embodiments, the antisense oligonucleotide of the invention is complementary to a target sequence in an A1CF mRNA.

The term “complementarity” describes the capacity for Watson-Crick base-pairing of nucleosides/nucleotides. Watson-Crick base pairs are cytosine-guanine (C-G) and adenine-thymine/uracil (A-T/U).

It will be understood that oligonucleotides may comprise nucleosides with modified nucleobases. For example 5-methyl cytosine (E) may be used in place of cytosine and 7-deaza-8-azaguanine (F) may be used in place of guanine. As such the term complementarity encompasses Watson-Crick base-pairing between non-modified and modified nucleobases (see for example Hirao et al., 2012, Accounts of Chemical Research, 45, 2055 and Bergstrom, 2009, Curr. Protoc. Nucleic Acid Chem., 37, 1.4.1). In particular, Watson-Crick base-pairing encompasses E-G, C-F and E-F base pairs.

The term “% complementary” as used herein, refers to the proportion of nucleotides (in percent) of a contiguous nucleotide sequence in a nucleic acid molecule (e.g. oligonucleotide) which across the contiguous nucleotide sequence, are complementary to a reference sequence (e.g. a target sequence). The percentage of complementarity is thus calculated by counting the number of aligned nucleobases that are complementary (i.e. form Watson Crick base pairs) between the two sequences (when aligned with the target sequence 5′-3′ and the contiguous nucleotide sequence from 3′-5′), dividing that number by the total number of nucleotides in the contiguous nucleotide sequence and multiplying by 100. In such a comparison a nucleobase/nucleotide which does not align (form a base pair) is termed a mismatch. Insertions and deletions are not allowed in the calculation of % complementarity of a contiguous nucleotide sequence. It will be understood that in determining complementarity, chemical modifications of the nucleobases are disregarded as long as the functional capacity of the nucleobase to form Watson Crick base pairing is retained (e.g. 5′-methyl cytosine is considered identical to a cytosine and 7-deaza-8-azaguanine is considered identical to a guanine for the purpose of calculating % complementarity).

The term “complementary” (such as in the phrase “the contiguous nucleotide sequence is complementary to a target sequence”) does not require 100% complementarity. Rather, within the present invention, the term “complementary” requires the contiguous nucleotide sequence to be at least 75% complementary, at least 80% complementary, at least 85% complementary, at least 90% complementary, at least 95% complementary or 100% complementary to the target sequence (such as a target sequence selected from SEQ ID NOs 31, 32, 33, 34, 35, 36, 37, 38, 39, 40 and 41). In some embodiments the antisense oligonucleotide, or contiguous nucleotide sequence thereof, is at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% complementary, or 100% complementary to the target sequence.

The term “fully complementary”, refers to 100% complementarity. In some embodiments the contiguous nucleotide sequence is fully complementary to the target sequence.

In some embodiments, the contiguous nucleotide sequences within the antisense oligonucleotides of the invention may include one, two, three or more mis-matches, wherein a mis-match is a nucleotide within the contiguous nucleotide sequence which does not base pair with its target.

In some embodiments, the contiguous nucleotide sequence is complementary to a target sequence selected from the group consisting of SEQ ID NOs 31, 32, 33, 34, 35, 36, 37, 38, 39, 40 and 41.

In some embodiments, the contiguous nucleotide sequence is complementary to SEQ ID NO 31. In some embodiments, the contiguous nucleotide sequence is complementary to SEQ ID NO 32. In some embodiments, the contiguous nucleotide sequence is complementary to SEQ ID NO 33. In some embodiments, the contiguous nucleotide sequence is complementary to SEQ ID NO 35. In some embodiments, the contiguous nucleotide sequence is complementary to SEQ ID NO 36. In some embodiments, the contiguous nucleotide sequence is complementary to SEQ ID NO 37. In some embodiments, the contiguous nucleotide sequence is complementary to SEQ ID NO 38. In some embodiments, the contiguous nucleotide sequence is complementary to SEQ ID NO 39. In some embodiments, the contiguous nucleotide sequence is complementary to SEQ ID NO 40. In some embodiments, the contiguous nucleotide sequence is complementary to SEQ ID NO 41.

In some embodiments, the contiguous nucleotide sequence is complementary to a target sequence within the sequence of SEQ ID NO 32 (positions 2181-2199 of the A1CF gene/pre-mRNA as depicted in SEQ ID NO 45). In some embodiments, the contiguous nucleotide sequence is complementary to SEQ ID NO 32. In some embodiments, the contiguous nucleotide sequence is at least 90%, at least 95%, or 100% complementary to SEQ ID NO 32. In some embodiments, the contiguous nucleotide sequence is 100% complementary to SEQ ID NO 32.

In some embodiments, the contiguous nucleotide sequence is complementary to a target sequence within the sequence of SEQ ID NO 42 (positions 6951-6970 of the A1CF gene/pre-mRNA as depicted in SEQ ID NO 45). In some embodiments, the contiguous nucleotide sequence is complementary to a target sequence selected from the group consisting of SEQ ID NOs 31, 36, 37 and 41. In some embodiments, the contiguous nucleotide sequence is at least 90%, at least 95%, or 100% complementary to a target sequence selected from the group consisting of SEQ ID NOs 31, 36, 37 and 41. In some embodiments, the contiguous nucleotide sequence is 100% complementary to a target sequence selected from the group consisting of SEQ ID NOs 31, 36, 37 and 41.

In some embodiments, the contiguous nucleotide sequence is complementary to a target sequence within the sequence of SEQ ID NO 43 (positions 16970-16989 of the A1CF gene/pre-mRNA as depicted in SEQ ID NO 45). In some embodiments, the contiguous nucleotide sequence is complementary to SEQ ID NO 33 or SEQ ID NO 38. In some embodiments, the contiguous nucleotide sequence is at least 90%, at least 95%, or 100% complementary to SEQ ID NO 33 or SEQ ID NO 38. In some embodiments, the contiguous nucleotide sequence is 100% complementary to SEQ ID NO 33 or SEQ ID NO 38.

In some embodiments, the contiguous nucleotide sequence is complementary to a target sequence within the sequence of SEQ ID NO 39 (positions 26358-26377 of the A1CF gene/pre-mRNA as depicted in SEQ ID NO 45). In some embodiments, the contiguous nucleotide sequence is complementary to SEQ ID NO 39. In some embodiments, the contiguous nucleotide sequence is at least 90%, at least 95%, or 100% complementary to SEQ ID NO 39. In some embodiments, the contiguous nucleotide sequence is 100% complementary to SEQ ID NO 39.

In some embodiments, the contiguous nucleotide sequence is complementary to a target sequence within the sequence of SEQ ID NO 34 (positions 38053-38071 of the A1CF gene/pre-mRNA as depicted in SEQ ID NO 45). In some embodiments, the contiguous nucleotide sequence is complementary to SEQ ID NO 34. In some embodiments, the contiguous nucleotide sequence is at least 90%, at least 95%, or 100% complementary to SEQ ID NO 34. In some embodiments, the contiguous nucleotide sequence is 100% complementary to SEQ ID NO 34.

In some embodiments, the contiguous nucleotide sequence is complementary to a target sequence within the sequence of SEQ ID NO 44 (positions 78973-78990 of the A1CF gene/pre-mRNA as depicted in SEQ ID NO 45). In some embodiments, the contiguous nucleotide sequence is complementary to SEQ ID NO 35 or SEQ ID NO 40. In some embodiments, the contiguous nucleotide sequence is at least 90%, at least 95%, or 100% complementary to SEQ ID NO 35 or SEQ ID NO 40. In some embodiments, the contiguous nucleotide sequence is 100% complementary to SEQ ID NO 35 or SEQ ID NO 40.

Hybridization

The terms “hybridizing” or “hybridizes” as used herein are to be understood as two nucleic acid strands (e.g. a contiguous nucleotide sequence and a target sequence) forming hydrogen bonds between base pairs on opposite strands thereby forming a duplex. The affinity of the binding between two nucleic acid strands is the strength of the hybridization. It is often described in terms of the melting temperature (Tₘ) defined as the temperature at which half of the oligonucleotides are duplexed with the target nucleic acid. At physiological conditions Tₘ is not strictly proportional to the affinity (Mergny and Lacroix, 2003, Oligonucleotides 13:515-537). The standard state Gibbs free energy ΔG° is a more accurate representation of binding affinity and is related to the dissociation constant (K_{d}) of the reaction by ΔG°=−RT ln(K_{d}), where R is the gas constant and T is the absolute temperature. Therefore, a very low ΔG° of the reaction between an oligonucleotide and the target nucleic acid reflects a strong hybridization between the oligonucleotide and target nucleic acid. ΔG° is the energy associated with a reaction where aqueous concentrations are 1M, the pH is 7, and the temperature is 37° C. The hybridization of oligonucleotides to a target nucleic acid is a spontaneous reaction and for spontaneous reactions ΔG° is less than zero. ΔG° can be measured experimentally, for example, by use of the isothermal titration calorimetry (ITC) method as described in Hansen et al., 1965, Chem. Comm. 36-38 and Holdgate et al., 2005, Drug Discov Today. The skilled person will know that commercial equipment is available for ΔG° measurements. ΔG° can also be estimated numerically by using the nearest neighbor model as described by SantaLucia, 1998, Proc Natl Acad Sci USA. 95: 1460-1465 using appropriately derived thermodynamic parameters described by Sugimoto et al., 1995, Biochemistry 34:11211-11216 and McTigue et al., 2004, Biochemistry 43:5388-5405.

In some embodiments, antisense oligonucleotides of the present invention hybridize to a target nucleic acid with estimated ΔG° values below −10 kcal for oligonucleotides that are 12-30 nucleotides in length.

In some embodiments the degree or strength of hybridization is measured by the standard state Gibbs free energy ΔG°. The oligonucleotides may hybridize to a target nucleic acid with estimated ΔG° values below the range of −10 kcal, such as below −15 kcal, such as below −20 kcal and such as below −25 kcal for oligonucleotides that are 8-30 nucleotides in length. In some embodiments the oligonucleotides hybridize to a target nucleic acid with an estimated ΔG° value of −10 to −60 kcal, such as −12 to −40, such as from −15 to −30 kcal, or −16 to −27 kcal such as −18 to −25 kcal.

Sequences

The term “sequence” as used herein refers to the order of nucleobases in a nucleic acid, such as an antisense oligonucleotide or a contiguous nucleotide sequence. When used to refer to a contiguous nucleotide sequence, an antisense oligonucleotide or an antisense oligonucleotide conjugate of the invention, the term “sequence” does not limit the form of the sugar moieties of the nucleic acid and does not limit the form of the internucleoside linkages of the nucleic acid. Thus, a given sequence of a contiguous nucleotide sequence, an antisense oligonucleotide or an antisense oligonucleotide conjugate may comprise any types of nucleoside sugar moieties (e.g. RNA, DNA, LNA, 2′-O-methyl-RNA, MOE-RNA, as described herein), in any combination, and may comprise any types of internucleoside linkages (e.g. phosphodiester, phosphorothioate, phosphorodithioate, as described herein).

In some embodiments, the sequence of the antisense oligonucleotide and/or the sequence of the contiguous nucleotide sequence comprises or consists of a sequence having at least 80% identity to any one of the following sequences:

| (SEQ ID NO 1) | |
|---|---|
| | AGGCATTATAGGTTTTTE, |
| (SEQ ID NO 2) | |
| | AAAAGTTTCTCAGATAGGE, |
| (SEQ ID NO 3) | |
| | GGACATGTTAATTTTACTT, |
| (SEQ ID NO 4) | |
| | TGAACTTGTTATATACETG, |
| (SEQ ID NO 5) | |
| | TAGAGTTTTGTGTGTET, |
| (SEQ ID NO 6) | |
| | GEATTATAGGTTTTTET, |
| (SEQ ID NO 7) | |
| | GGCATTATAGGTTTTTE, |
| (SEQ ID NO 8) | |
| | GGEATTATAGGTTTTTE, |
| (SEQ ID NO 9) | |
| | TGGACAUGTTAATTTTAE, |
| (SEQ ID NO 10) | |
| | GGACATGUTAATTTTACTT, |
| (SEQ ID NO 11) | |
| | GTTTTAAGATTGTGGTGTTT, |
| (SEQ ID NO 12) | |
| | TAGAGTTTUGTGTGTET, |
| (SEQ ID NO 13) | |
| | TAGAGTTTTGTGUGTET, |
| (SEQ ID NO 14) | |
| | ATAGAFTTTTGTGTGTE, |
| (SEQ ID NO 15) | |
| | AAAAFTTTCTCAGATAGGE, |
| (SEQ ID NO 16) | |
| | AAAAGTTTCTCAFATAGGE, |
| (SEQ ID NO 17) | |
| | AAAAGTTUCTCAGATAGGE, |
| (SEQ ID NO 18) | |
| | AGGCATTATAGGUTTTTE, |
| (SEQ ID NO 19) | |
| | AGGCAUTATAGGTTTTTE, |
| (SEQ ID NO 20) | |
| | GEATTATAGGTTTTTCTE, |
| (SEQ ID NO 21) | |
| | GEATTATAGGTTTTUCTE, |
| (SEQ ID NO 22) | |
| | GEATTATAGGTTTUTCTE, |
| (SEQ ID NO 23) | |
| | GEATTATAGGTTUTTCTE, |
| (SEQ ID NO 24) | |
| | GEATTATAGGTTUTTET, |
| (SEQ ID NO 25) | |
| | GEATTATAGGTUTTTET, |
| (SEQ ID NO 26) | |
| | GEATTATAGGUTTTTCTE, |
| (SEQ ID NO 27) | |
| | GEATTATAGGUTTTTET, |
| (SEQ ID NO 28) | |
| | GEATTAUAGGTTTTTET, |
| (SEQ ID NO 29) | |
| | GEATUATAGGTTTTTET |
| | and |
| (SEQ ID NO 30) | |
| | GEAUTATAGGTTTTTET. |

In some embodiments, the sequence of the antisense oligonucleotide comprises a sequence having at least 80% identity to any one of SEQ ID NOs 1, 2, 3, 4, 5, 6, 7, 8, 9, 10, 11, 12, 13, 14, 15, 16, 17, 18, 19, 20, 21, 22, 23, 24, 25, 26, 27, 28, 29 and 30. In some embodiments, the sequence of the antisense oligonucleotide consists of a sequence having at least 80% identity to any one of SEQ ID NOs 1, 2, 3, 4, 5, 6, 7, 8, 9, 10, 11, 12, 13, 14, 15, 16, 17, 18, 19, 20, 21, 22, 23, 24, 25, 26, 27, 28, 29 and 30. In some embodiments, the sequence of the contiguous nucleotide sequence comprises a sequence having at least 80% identity to any one of SEQ ID NOs 1, 2, 3, 4, 5, 6, 7, 8, 9, 10, 11, 12, 13, 14, 15, 16, 17, 18, 19, 20, 21, 22, 23, 24, 25, 26, 27, 28, 29 and 30. In some embodiments, the sequence of the contiguous nucleotide sequence consists of a sequence having at least 80% identity to any one of SEQ ID NOs 1, 2, 3, 4, 5, 6, 7, 8, 9, 10, 11, 12, 13, 14, 15, 16, 17, 18, 19, 20, 21, 22, 23, 24, 25, 26, 27, 28, 29 and 30.

In some embodiments, the sequence of the antisense oligonucleotide and/or the sequence of the contiguous nucleotide sequence comprises or consists of a sequence having at least 85%, at least 90%, at least 95% or 100% identity to any one of SEQ ID NOs 1, 2, 3, 4, 5, 6, 7, 8, 9, 10, 11, 12, 13, 14, 15, 16, 17, 18, 19, 20, 21, 22, 23, 24, 25, 26, 27, 28, 29 and 30. In some embodiments, the sequence of the antisense oligonucleotide and/or the sequence of the contiguous nucleotide sequence comprises or consists of a sequence having at least 85%, at least 86%, at least 87%, at least 88%, at least 89%, at least 90%, at least 91%, at least 92%, at least 93%, at least 94%, at least at least 95%, at least 96%, at least 97%, at least 98%, at least 99% or 100% identity to any one of SEQ ID NOs 1, 2, 3, 4, 5, 6, 7, 8, 9, 10, 11, 12, 13, 14, 15, 16, 17, 18, 19, 20, 21, 22, 23, 24, 25, 26, 27, 28, 29 and 30.

In some embodiments, the sequence of the antisense oligonucleotide comprises a sequence having at least 85%, at least 86%, at least 87%, at least 88%, at least 89%, at least 90%, at least 91%, at least 92%, at least 93%, at least 94%, at least at least 95%, at least 96%, at least 97%, at least 98%, at least 99% or 100% identity to any one of SEQ ID NOs 1, 2, 3, 4, 5, 6, 7, 8, 9, 10, 11, 12, 13, 14, 15, 16, 17, 18, 19, 20, 21, 22, 23, 24, 25, 26, 27, 28, 29 and 30. In some embodiments, the sequence of the antisense oligonucleotide consists of a sequence having at least 85%, at least 86%, at least 87%, at least 88%, at least 89%, at least 90%, at least 91%, at least 92%, at least 93%, at least 94%, at least at least 95%, at least 96%, at least 97%, at least 98%, at least 99% or 100% identity to any one of SEQ ID NOs 1, 2, 3, 4, 5, 6, 7, 8, 9, 10, 11, 12, 13, 14, 15, 16, 17, 18, 19, 20, 21, 22, 23, 24, 25, 26, 27, 28, 29 and 30. In some embodiments, the sequence of the contiguous nucleotide sequence comprises a sequence having at least 85%, at least 86%, at least 87%, at least 88%, at least 89%, at least 90%, at least 91%, at least 92%, at least 93%, at least 94%, at least at least 95%, at least 96%, at least 97%, at least 98%, at least 99% or 100% identity to any one of SEQ ID NOs 1, 2, 3, 4, 5, 6, 7, 8, 9, 10, 11, 12, 13, 14, 15, 16, 17, 18, 19, 20, 21, 22, 23, 24, 25, 26, 27, 28, 29 and 30. In some embodiments, the sequence of the contiguous nucleotide sequence consists of a sequence having at least 85%, at least 86%, at least 87%, at least 88%, at least 89%, at least 90%, at least 91%, at least 92%, at least 93%, at least 94%, at least at least 95%, at least 96%, at least 97%, at least 98%, at least 99% or 100% to any one of SEQ ID NOs 1, 2, 3, 4, 5, 6, 7, 8, 9, 10, 11, 12, 13, 14, 15, 16, 17, 18, 19, 20, 21, 22, 23, 24, 25, 26, 27, 28, 29 and 30.

In some embodiments, the sequence of the antisense oligonucleotide and/or the sequence of the contiguous nucleotide sequence comprises or consists of a sequence having at least 80% identity to any one of SEQ ID NOs 1, 2, 3, 4, 5, 6, 7, 8, 9, 10, 11, 12, 13 and 14.

In some embodiments, the sequence of the antisense oligonucleotide comprises a sequence having at least 80% identity to any one of SEQ ID NOs 1, 2, 3, 4, 5, 6, 7, 8, 9, 10, 11, 12, 13 and 14. In some embodiments, the sequence of the antisense oligonucleotide consists of a sequence having at least 80% identity to any one of SEQ ID NOs 1, 2, 3, 4, 5, 6, 7, 8, 9, 10, 11, 12, 13 and 14. In some embodiments, the sequence of the contiguous nucleotide sequence comprises a sequence having at least 80% identity to any one of SEQ ID NOs 1, 2, 3, 4, 5, 6, 7, 8, 9, 10, 11, 12, 13 and 14. In some embodiments, the sequence of the contiguous nucleotide sequence consists of a sequence having at least 80% identity to any one of SEQ ID NOs 1, 2, 3, 4, 5, 6, 7, 8, 9, 10, 11, 12, 13 and 14.

In some embodiments, the sequence of the antisense oligonucleotide and/or the sequence of the contiguous nucleotide sequence comprises or consists of a sequence having at least 85%, at least 86%, at least 87%, at least 88%, at least 89%, at least 90%, at least 91%, at least 92%, at least 93%, at least 94%, at least at least 95%, at least 96%, at least 97%, at least 98%, at least 99% or 100% identity to any one of SEQ ID NOs 1, 2, 3, 4, 5, 6, 7, 8, 9, 10, 11, 12, 13 and 14. In some embodiments, the sequence of the antisense oligonucleotide comprises a sequence having at least 85%, at least 86%, at least 87%, at least 88%, at least 89%, at least 90%, at least 91%, at least 92%, at least 93%, at least 94%, at least at least 95%, at least 96%, at least 97%, at least 98%, at least 99% or 100% identity to any one of SEQ ID NOs 1, 2, 3, 4, 5, 6, 7, 8, 9, 10, 11, 12, 13 and 14. In some embodiments, the sequence of the antisense oligonucleotide consists of a sequence having at least 85%, at least 86%, at least 87%, at least 88%, at least 89%, at least 90%, at least 91%, at least 92%, at least 93%, at least 94%, at least at least 95%, at least 96%, at least 97%, at least 98%, at least 99% or 100% identity to any one of SEQ ID NOs 1, 2, 3, 4, 5, 6, 7, 8, 9, 10, 11, 12, 13 and 14. In some embodiments, the sequence of the contiguous nucleotide sequence comprises a sequence having at least 85%, at least 86%, at least 87%, at least 88%, at least 89%, at least 90%, at least 91%, at least 92%, at least 93%, at least 94%, at least at least 95%, at least 96%, at least 97%, at least 98%, at least 99% or 100% identity to any one of SEQ ID NOs 1, 2, 3, 4, 5, 6, 7, 8, 9, 10, 11, 12, 13 and 14. In some embodiments, the sequence of the contiguous nucleotide sequence consists of a sequence having at least 85%, at least 86%, at least 87%, at least 88%, at least 89%, at least 90%, at least 91%, at least 92%, at least 93%, at least 94%, at least at least 95%, at least 96%, at least 97%, at least 98%, at least 99% or 100% identity to any one of SEQ ID NOs 1, 2, 3, 4, 5, 6, 7, 8, 9, 10, 11, 12, 13 and 14.

In some embodiments, the sequence of the antisense oligonucleotide and/or the sequence of the contiguous nucleotide sequence comprises or consists of a sequence having at least 80% identity to any one of SEQ ID NOs 1, 2, 3, 4 and 5.

In some embodiments, the sequence of the antisense oligonucleotide comprises a sequence having at least 80% identity to any one of SEQ ID NOs 1, 2, 3, 4 and 5. In some embodiments, the sequence of the antisense oligonucleotide consists of a sequence having at least 80% identity to any one of SEQ ID NOs 1, 2, 3, 4 and 5. In some embodiments, the sequence of the contiguous nucleotide sequence comprises a sequence having at least 80% identity to any one of SEQ ID NOs 1, 2, 3, 4 and 5. In some embodiments, the sequence of the contiguous nucleotide sequence consists of a sequence having at least 80% identity to any one of SEQ ID NOs 1, 2, 3, 4 and 5.

In some embodiments, the sequence of the antisense oligonucleotide and/or the sequence of the contiguous nucleotide sequence comprises or consists of a sequence having at least 85%, at least 86%, at least 87%, at least 88%, at least 89%, at least 90%, at least 91%, at least 92%, at least 93%, at least 94%, at least at least 95%, at least 96%, at least 97%, at least 98%, at least 99% or 100% identity to any one of SEQ ID NOs 1, 2, 3, 4 and 5. In some embodiments, the sequence of the antisense oligonucleotide comprises a sequence having at least 85%, at least 86%, at least 87%, at least 88%, at least 89%, at least 90%, at least 91%, at least 92%, at least 93%, at least 94%, at least at least 95%, at least 96%, at least 97%, at least 98%, at least 99% or 100% identity to any one of SEQ ID NOs 1, 2, 3, 4 and 5. In some embodiments, the sequence of the antisense oligonucleotide consists of a sequence having at least 85%, at least 86%, at least 87%, at least 88%, at least 89%, at least 90%, at least 91%, at least 92%, at least 93%, at least 94%, at least at least 95%, at least 96%, at least 97%, at least 98%, at least 99% or 100% identity to any one of SEQ ID NOs 1, 2, 3, 4 and 5. In some embodiments, the sequence of the contiguous nucleotide sequence comprises a sequence having at least 85%, at least 86%, at least 87%, at least 88%, at least 89%, at least 90%, at least 91%, at least 92%, at least 93%, at least 94%, at least at least 95%, at least 96%, at least 97%, at least 98%, at least 99% or 100% identity to any one of SEQ ID NOs 1, 2, 3, 4 and 5. In some embodiments, the sequence of the contiguous nucleotide sequence consists of a sequence having at least 85%, at least 86%, at least 87%, at least 88%, at least 89%, at least 90%, at least 91%, at least 92%, at least 93%, at least 94%, at least at least 95%, at least 96%, at least 97%, at least 98%, at least 99% or 100% identity to any one of SEQ ID NOs 1, 2, 3, 4 and 5.

Identity

The term “identity” as used herein, refers to the proportion of nucleotides (expressed in percent) of a contiguous nucleotide sequence in a nucleic acid molecule (e.g. oligonucleotide) which, across the contiguous nucleotide sequence, are identical to a reference sequence (e.g. a target sequence).

The percentage of identity is thus calculated by counting the number of aligned nucleobases that are identical (a Match) between two sequences (in the contiguous nucleotide sequence and in the reference sequence), dividing that number by the total number of nucleotides in the contiguous nucleotide sequence and multiplying by 100. Therefore, Percentage of Identity=(Matches×100)/Length of aligned region (e.g. the contiguous nucleotide sequence). Insertions and deletions are not allowed in the calculation the percentage of identity of a contiguous nucleotide sequence. It will be understood that in determining identity, chemical modifications of the nucleobases are disregarded as long as the functional capacity of the nucleobase to form Watson Crick base pairing is retained (e.g. 5-methyl cytosine is considered identical to a cytosine and 7-deaza-8-azaguanine is considered identical to a guanine for the purpose of calculating % identity).

Sugar Modifications

In some embodiments, the antisense oligonucleotide of the invention comprises one or more sugar modified nucleosides. In other words, the antisense oligonucleotides of the invention may comprise one or more nucleosides which have a modified sugar moiety, i.e. a modification of the sugar moiety when compared to the ribose sugar moiety found in DNA and RNA.

Numerous nucleosides with modification of the ribose sugar moiety have been made, primarily with the aim of improving certain properties of oligonucleotides, such as affinity and/or nuclease resistance.

Such modifications include those where the ribose ring structure is modified, e.g. by replacement with a hexose ring (HNA), or a bicyclic ring, which typically have a biradicle bridge between the C2 and C4 carbons on the ribose ring (LNA), or an unlinked ribose ring which typically lacks a bond between the C2 and C3 carbons (e.g. UNA). Other sugar modified nucleosides include, for example, bicyclohexose nucleic acids (WO 2011/017521) or tricyclic nucleic acids (WO 2013/154798). Modified nucleosides also include nucleosides where the sugar moiety is replaced with a non-sugar moiety, for example in the case of peptide nucleic acids (PNA), or morpholino nucleic acids.

Sugar modifications also include modifications made via altering the substituent groups on the ribose ring to groups other than hydrogen, or the 2′-OH group naturally found in DNA and RNA nucleosides. Substituents may, for example be introduced at the 2′, 3′, 4′ or 5′ positions.

In some embodiments, the sugar modified nucleosides are independently selected from the group consisting of 2′-O-methyl-RNA, 2′-O-methoxyethyl-RNA (MOE-RNA) and LNA nucleosides.

In some embodiments, each nucleotide in the antisense oligonucleotide of the invention that does not comprise a sugar modified nucleoside comprises a DNA nucleoside.

2′ Sugar Modified Nucleosides

A 2′ sugar modified nucleoside is a nucleoside which has a substituent other than H or —OH at the 2′ position (2′ substituted nucleoside) or comprises a 2′ linked biradicle capable of forming a bridge between the 2′ carbon and a second carbon in the ribose ring, such as LNA (2′-4′ biradicle bridged) nucleosides.

Indeed, much focus has been spent on developing 2′ sugar substituted nucleosides, and numerous 2′ substituted nucleosides have been found to have beneficial properties when incorporated into oligonucleotides. For example, the 2′ modified sugar may provide enhanced binding affinity and/or increased nuclease resistance to the oligonucleotide. Examples of 2′ substituted modified nucleosides are 2′-O-alkyl-RNA, 2′-O-methyl-RNA (2′OMe). 2′-alkoxy-RNA, 2′-O-methoxyethyl-RNA (MOE), 2′-amino-DNA, 2′-Fluoro-RNA, and 2′-F-ANA nucleoside. For further examples, please see e.g. Freier & Altmann; Nucl. Acid Res., 1997, 25, 4429-4443 and Uhlmann; Curr. Opinion in Drug Development 2000, 3(2), 203-213, and Deleavey and Damha, Chemistry and Biology 2012, 19, 937. Below are illustrations of some 2′ substituted modified nucleosides.

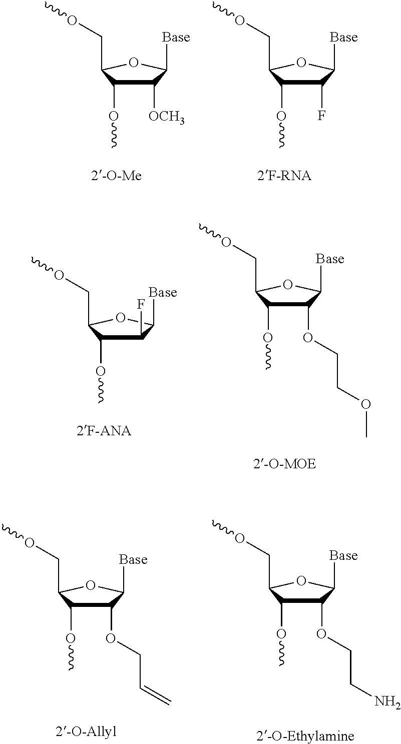

In some embodiments, the antisense oligonucleotide comprises one or more 2′ sugar modified nucleosides.

In some embodiments, the antisense oligonucleotide of the invention comprises one or more 2′-O-methyl-RNA nucleosides.

In some embodiments, the antisense oligonucleotide of the invention comprises one or more 2′-O-methoxyethyl-RNA (MOE-RNA) nucleosides.

Locked Nucleic Acid Nucleosides (LNA Nucleoside)

In some embodiments, the antisense oligonucleotide comprises one or more LNA nucleosides.

A “LNA nucleoside” is a 2′-modified nucleoside which comprises a biradical linking the C2′ and C4′ of the ribose sugar ring of said nucleoside (also referred to as a “2′-4′ bridge”), which restricts or locks the conformation of the ribose ring. These nucleosides are also termed bridged nucleic acid or bicyclic nucleic acid (BNA) in the literature. The locking of the conformation of the ribose is associated with an enhanced affinity of hybridization (duplex stabilization) when the LNA is incorporated into an oligonucleotide for a complementary RNA or DNA molecule. This can be routinely determined by measuring the melting temperature of the oligonucleotide/complement duplex.

Non limiting, exemplary LNA nucleosides are disclosed in WO 99/014226, WO 00/66604, WO 98/039352, WO 2004/046160, WO 00/047599, WO 2007/134181, WO 2010/077578, WO 2010/036698, WO 2007/090071, WO 2009/006478, WO 2011/156202, WO 2008/154401, WO 2009/067647, WO 2008/150729, Morita et al., Bioorganic & Med. Chem. Lett., 12, 73-76, Seth et al., J. Org. Chem., 2010, Vol 75(5) pp. 1569-81, Mitsuoka et al., Nucleic Acids Research, 2009, 37(4), 1225-1238, and Wan and Seth, J. Medical Chemistry, 2016, 59, 9645-9667.

Further non limiting, exemplary LNA nucleosides are disclosed in Scheme 1.

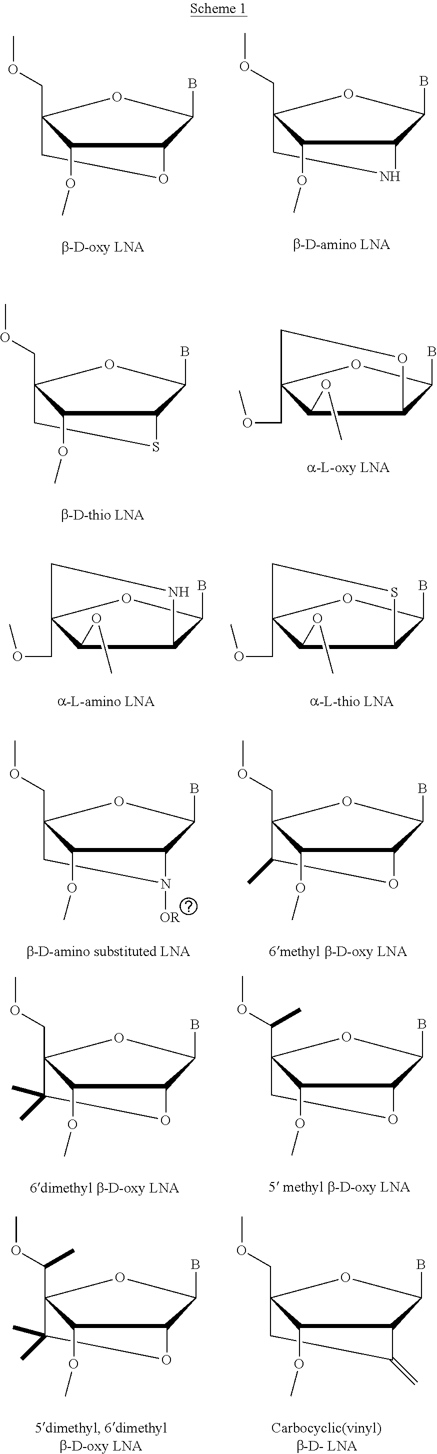

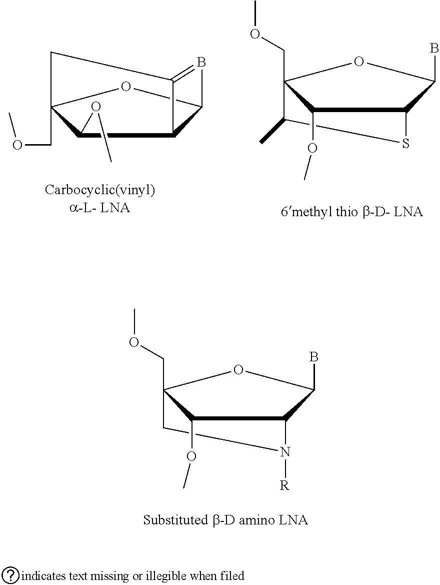

Particular LNA nucleosides are beta-D-oxy-LNA, 6′-methyl-beta-D-oxy LNA such as (S)-6′-methyl-beta-D-oxy-LNA (ScET) and ENA.

In some embodiments, the antisense oligonucleotide comprises one or more LNA beta-D-oxy-LNA nucleosides.

Modified Internucleoside Linkages

In some embodiments, the antisense oligonucleotides of the invention may comprise one or more modified internucleoside linkages.

The term “modified internucleoside linkage” is defined, as generally understood by the skilled person, as linkages other than phosphodiester (PO) linkages that covalently couple two nucleosides together.

In some embodiments, all internucleoside linkages in the antisense oligonucleotide are modified internucleoside linkages.

In some embodiments, each modified internucleoside linkage is independently selected from the group consisting of phosphorothioate internucleoside linkages and phosphorodithioate internucleoside linkages. Phosphorothioate internucleoside linkages and phosphorodithioate internucleoside linkages are useful in that they may render the antisense oligonucleotide more resistant to degradation by nucleases. In a phosphorothioate internucleoside linkage, relative to a naturally occurring phosphodiester internucleoside linkage, one of the oxygen atoms in the phosphate group that is not bonded to a carbon of a nucleoside sugar moiety is replaced with a sulphur atom. In a phosphorodithioate internucleoside linkage, each of the two oxygen atoms in the phosphate group that is not bonded to a carbon of a nucleoside sugar moiety is replaced with a sulphur atom. Thus, where a phosphodiester bond may be represented by the formula —O—P(O)₂—O—, a phosphorothioate internucleoside linkage may be represented by the formula —O—P(O,S)—O—, and a phosphorodithioate internucleoside linkage may be represented by the formula —O—P(S)₂—O—.

Regions D′ and D″

The antisense oligonucleotides of the invention may in some embodiments comprise or consist of the contiguous nucleotide sequences of the oligonucleotides, which are complementary to the target nucleic acid, and further 5′ and/or 3′ nucleosides. The further 5′ and/or 3′ nucleosides may or may not be complementary, such as fully complementary, to the target nucleic acid. Such further 5′ and/or 3′ nucleosides may be referred to as region D′ and D″ herein.

The addition of region D′ or D″ may be used for the purpose of joining the contiguous nucleotide sequence, such as the mixmer or totalmer, to a conjugate moiety or another functional group. When used for joining the contiguous nucleotide sequence with a conjugate moiety is can serve as a biocleavable linker. Alternatively, it may be used to provide exonucleoase protection or for ease of synthesis or manufacture.

Region D′ or D″ may independently comprise or consist of 1, 2, 3, 4 or 5 additional nucleotides, which may be complementary or non-complementary to the target nucleic acid. The nucleotide adjacent to the F or F′ region is not a sugar-modified nucleotide, such as a DNA or RNA or base modified versions of these. The D′ or D′ region may serve as a nuclease susceptible biocleavable linker (see definition of linkers). In some embodiments the additional 5′ and/or 3′ end nucleotides are linked with phosphodiester linkages, and are DNA or RNA. Nucleotide based biocleavable linkers suitable for use as region D′ or D″ are disclosed in WO2014/076195, which include by way of example a phosphodiester linked DNA dinucleotide. The use of biocleavable linkers in poly-oligonucleotide constructs is disclosed in WO2015/113922, where they are used to link multiple antisense constructs within a single oligonucleotide.

In one embodiment, the antisense oligonucleotides of the invention comprise a region D′ and/or D″ in addition to the contiguous nucleotide sequence.

In some embodiments, the internucleoside linkage positioned between region D′ or D″ and the contiguous nucleotide sequence is a phosphodiester linkage.

Compounds

The term “compound” is used herein to refer to the combination of sequence (i.e. the order of nucleobases), sugar moieties and internucleoside linkages in a given nucleic acid, such as an antisense oligonucleotide of the invention. For a given compound, the nucleobase, the type of sugar moiety and the internucleoside linkages of each nucleotide are specified. Thus, the order of sugar moieties and internucleoside linkages is also specified in a given compound. However, unless otherwise indicated, a compound may comprise other elements in addition to the specified sequence, sugar moieties and internucleoside linkages.

In some embodiments, the term “antisense oligonucleotide” is interchangeable with the term “compound”. In other words, in some embodiments, an antisense oligonucleotide of the invention is a compound of the invention, and vice versa.

Specific compounds are referred to herein using a compound identifier number (CMP ID NO) of the form X_Y, wherein X and Y are each a number. For each CMP ID NO, X is the number of the SEQ ID NO that corresponds to the sequence of the compound. For example, the compound designated CMP ID NO 2_1 has the same nucleobase sequence as SEQ ID NO 2.

In some embodiments, the antisense oligonucleotide comprises any one of CMP ID NOs 1_1, 2_1, 3_1, 4_1, 4_2, 5_1, 5_2, 6_1, 7_1, 7_2, 8_1, 2_2, 9_1, 10_1, 11_1, 4_3, 12_1, 5_3, 13_1, 14_1, 15_1, 16_1, 16_2, 16_3, 2_3, 2_4, 2_5, 2_6, 2_7, 2_8, 2_9, 2_10, 2_11, 2_12, 17_1, 18_1, 19_1, 20_1, 20_2, 20_3, 20_4, 20_5, 20_6, 6_2, 6_3, 6_4, 6_5, 6_6, 6_7, 6_8, 6_9, 6_10, 6_11, 6_12, 21_1, 22_1, 23_1, 24_1, 25_1, 25_2, 26_1, 27_1, 27_2, 28_1, 29_1, 30_1, 11_2 and 11_3. In some embodiments, the antisense oligonucleotide consists of any one of CMP ID NOs NOs 1_1, 2_1, 3_1, 4_1, 4_2, 5_1, 5_2, 6_1, 7_1, 7_2, 8_1, 2_2, 9_1, 101, 11_1, 4_3, 12_1, 5_3, 13_1, 14_1, 15_1, 16_1, 16_2, 16_3, 2_3, 2_4, 2_5, 2_6, 2_7, 2_8, 2_9, 2_10, 2_11, 2_12, 171, 18_1, 19_1, 20_1, 20_2, 203, 20_4, 20_5, 20_6, 6_2, 6_3, 6_4, 6_5, 6_6, 6_7, 6_8, 6_9, 6_10, 6_11, 6_12, 21_1, 22_1, 23_1, 24_1, 25_1, 25_2, 26_1, 27_1, 27_2, 28_1, 29_1, 30_1, 11_2 and 11_3.

In some embodiments the antisense oligonucleotide comprises any one of CMP ID NOs 1_1, 2_1, 3_1, 4_1, 4_2, 5_1, 5_2, 6_1, 7_1, 7_2, 8_1, 2_2, 9_1, 10_1, 11_1, 4_3, 12_1, 5_3, 13_1 and 14_1. In some embodiments the antisense oligonucleotide consists of any one of CMP ID NOs 1_1, 2_1, 3_1, 4_1, 4_2, 5_1, 5_2, 6_1, 7_1, 7_2, 8_1, 2_2, 9_1, 10_1, 11_1, 4_3, 12_1, 5_3, 13_1 and 14_1.

In some embodiments, the antisense oligonucleotide comprises or consists of any one of CMP ID NOs 1_1, 2_1, 3_1, 4_1, 4_2, 5_1 and 5_2. In some embodiments, the antisense oligonucleotide comprises any one of CMP ID NOs 1_1, 2_1, 3_1, 4_1, 4_2, 5_1 and 5_2. In some embodiments, the antisense oligonucleotide consists of any one of CMP ID NOs 1_1, 2_1, 3_1, 4_1, 4_2, 5_1 and 5_2.

The invention also provides an antisense oligonucleotide comprising or consisting of a compound selected from the following:

(a)
[LR](A)[sP].[LR](G)[sP].[dR](G)[sP].[dR](C)[sP].[LR](A)[sP].[dR](T)[sP].[dR](T)[sP].
[dR](A)[sP].[dR](T)[sP].[dR](A)[sP].[dR](G)[sP].[dR](G)[sP].[dR](T)[sP].[dR](T)[sP].
[dR](T)[sP].[LR](T)[sP].[LR](T)[sP].[LR]([5meC]) (SEQ ID NO 1, CMP ID NO 1_1),
(b)
[LR](A)[PS2].[LR](A)[sP].[dR](A)[sP].[mR](A)[sP].[LR](G)[sP].[dR](T)[sP].[dR](T)[sP].
[dR](T)[sP].[dR](C)[sP].[dR](T)[sP].[dR](C)[sP].[dR](A)[sP].[dR](G)[sP].[dR](A)[sP].
[dR](T)[sP].[dR](A)[sP].[mR](G)[sP].[LR](G)[PS2].[LR]([5meC])
(SEQ ID NO 2, CMP ID NO 2_1),
(c)
[LR](G)[P].[LR](G)[sP].[mR](A)[sP].[dR](C)[sP].[dR](A)[P].[dR](T)[sP].[LR](G)[sP].
[dR](T)[sP].[dR](T)[sP].[dR](A)[sP].[dR](A)[sP].[dR](T)[sP].[dR](T)[sP].[dR](T)[sP].[dR]
(T)[sP].[dR](A)[sP].[dR](C)[sP].[LR](T)[PS2].[LR](T) (SEQ ID NO 3, CMP ID NO 3_1),
(d)
[LR](T)[PS2].[LR](G)[sP].[LR](A)[sP].[dR](A)[sP].[dR](C)[sP].[dR](T)[sP].[dR](T)[sP].
[dR](G)[sP].[dR](T)[sP].[dR](T)[sP].[dR](A)[sP].[dR](T)[sP].[dR](A)[sP].[dR](T)[sP].
[dR](A)[sP].[dR](C)[sP].[LR]([5meC])[PS2].[LR](T)[PS2].[LR](G)
(SEQ ID NO 4, CMP ID NO 4_1),
(e)
[LR](T)[PS2].[LR](G)[sP].[LR](A)[sP].[mR](A)[sP].[dR](C)[sP].[dR](T)[sP].[dR](T)[sP].
[dR](G)[sP].[dR](T)[sP].[dR](T)[sP].[dR](A)[sP].[dR](T)[sP].[dR](A)[sP].[dR](T)[sP].
[dR](A)[sP].[dR](C)[sP].[LR]([5meC])[sP].[LR](T)[PS2].[LR](G)
(SEQ ID NO 4, CMP ID NO 4_2),
(f)
[LR](T)[PS2].[LR](A)[PS2].[dR](G)[sP].[dR](A)[sP].[LR](G)[sP].[dR](T)[sP].[dR](T)[sP].
[dR](T)[sP].[dR](T)[sP].[dR](G)[sP].[dR](T)[sP].[dR](G)[sP].[dR](T)[sP].[dR](G)[sP].
[dR](T)[sP].[MOE]([5meC])[sP].[MOE](T) (SEQ ID NO 5, CMP ID NO 5_1),
(g)
[LR](T)[PS2].[LR](A)[sP].[dR](G)[sP].[dR](A)[sP].[LR](G)[sP].[dR](T)[sP].[dR](T)[sP].
[dR](T)[sP].[dR](T)[sP].[dR](G)[sP].[dR](T)[sP].[dR](G)[sP].[dR](T)[sP].[dR](G)[sP].
[dR](T)[sP].[MOE]([5meC])[sP].[MOE](T) (SEQ ID NO 5, CMP ID NO 5_2),
(h)
[LR](G)[sP].[LR]([5meC])[sP].[dR](A)[sP].[dR](T)[sP].[dR](T)[sP].[dR](A)[sP].[dR](T)
[sP].[dR](A)[sP].[dR](G)[sP].[dR](G)[sP].[dR](T)[sP].[dR](T)[sP].[dR](T)[sP].[LR](T)
[sP].[LR](T)[sP].[LR]([5meC])[sP].[LR](T) (SEQ ID NO 6, CMP ID NO 6_1),
(i)
[MOE](G)[sP].[LR](G)[sP].[dR](C)[sP].[LR](A)[sP].[dR](T)[sP].[dR](T)[sP].[dR](A)[sP].
[dR](T)[sP].[dR](A)[sP].[dR](G)[sP].[dR](G)[sP].[dR](T)[sP].[dR](T)[sP].[LR](T)[sP].
[MOE](T)[sP].[MOE](T)[sP].[MOE]([5meC]) (SEQ ID NO 7, CMP ID NO 7_1),
(j)
[LR](G)[sP].[LR](G)[sP].[dR](C)[sP].[LR](A)[sP].[dR](T)[sP].[dR](T)[sP].[dR](A)[sP].
[dR](T)[sP].[mR](A)[sP].[dR](G)[sP].[dR](G)[sP].[dR](T)[sP].[dR](T)[sP].[dR](T)[sP].
[LR](T)[sP].[LR](T)[sP].[LR]([5meC]) (SEQ ID NO 7, CMP ID NO 7_2),
(k)
[MOE](G)[sP].[MOE](G)[sP].[MOE]([5meC])[sP].[LR](A)[PS2].[dR](T)[sP].[dR](T)[sP].
[dR](A)[sP].[dR](T)[sP].[mR](A)[sP].[dR](G)[sP].[dR](G)[sP].[dR](T)[sP].[dR](T)[sP].
[LR](T)[PS2].[LR](T)[PS2].[LR](T)[PS2].[MOE]([5meC])
(SEQ ID NO 8, CMP ID NO 8_1),
(l)
[LR](A)[PS2].[LR](A)[PS2].[dR](A)[sP].[mR](A)[sP].[LR](G)[sP].[dR](T)[sP].[dR](T)[sP].
[dR](T)[sP].[dR](C)[sP].[dR](T)[sP].[dR](C)[sP].[dR](A)[sP].[dR](G)[sP].[dR](A)[sP].
[dR](T)[sP].[dR](A)[sP].[dR](G)[sP].[LR](G)[PS2].[LR]([5meC])
(SEQ ID NO 2, CMP ID NO 2_2),
(m)
[LR](T)[PS2].[LR](G)[sP].[dR](G)[sP].[LR](A)[sP].[dR](C)[sP].[dR](A)[sP].[mR](U)[sP].
[dR](G)[sP].[dR](T)[sP].[LR](T)[sP].[dR](A)[sP].[dR](A)[sP].[dR](T)[sP].[dR](T)[sP].
[dR](T)[sP].[dR](T)[sP].[LR](A)[PS2].[LR]([5meC]) (SEQ ID NO 9, CMP ID NO 9_1),
(n)
[LR](G)[PS2].[LR](G)[PS2].[dR](A)[sP].[dR](C)[sP].[dR](A)[sP].[dR](T)[P].[LR](G)[sP].
[mR](U)[sP].[dR](T)[sP].[mR](A)[sP].[dR](A)[sP].[dR](T)[sP].[dR](T)[sP].[dR](T)[sP].
[dR](T)[sP].[dR](A)[sP].[dR](C)[sP].[LR](T)[PS2].[LR](T)
(SEQ ID NO 10, CMP ID NO 10_1),
(o)
[LR](G)[sP].[LR](T)[sP].[LR](T)[sP].[dR](T)[sP].[dR](T)[sP].[mR](A)[sP].[dR](A)[sP].
[dR](G)[sP].[dR](A)[sP].[dR](T)[P].[dR](T)[sP].[dR](G)[sP].[dR](T)[sP].[dR](G)[sP].
[dR](G)[sP].[dR](T)[sP].[dR](G)[sP].[LR](T)[sP].[LR](T)[PS2].[LR](T)
(SEQ ID NO 11, CMP ID NO 11_1),
(p)
[LR](T)[PS2].[LR](G)[sP].[LR](A)[P].[dR](A)[sP].[dR](C)[sP].[dR](T)[sP].[dR](T)[sP].
[dR](G)[sP].[dR](T)[sP].[dR](T)[sP].[dR](A)[sP].[dR](T)[sP].[dR](A)[sP].[dR](T)[sP].
[dR](A)[sP].[dR](C)[sP].[LR]([5meC])[sP].[LR](T)[PS2].[LR](G)
(SEQ ID NO 4, CMP ID NO 4_3),
(q)
[LR](T)[PS2].[LR](A)[PS2].[dR](G)[sP].[dR](A)[sP].[LR](G)[sP].[dR](T)[sP].[dR](T)
[sP].[dR](T)[sP].[mR](U)[sP].[dR](G)[sP].[dR](T)[sP].[dR](G)[sP].[dR](T)[sP].[dR](G)
[sP].[dR](T)[sP].[MOE]([5meC])[sP].[MOE](T) (SEQ ID NO 12, CMP ID NO 12_1),
(r)
[MOE](T)[P].[MOE](A)[sP].[MOE](G)[sP].[MOE](A)[sP].[LR](G)[PS2].[dR](T)[sP].[dR]
(T)[sP].[dR](T)[sP].[dR](T)[sP].[dR](G)[sP].[dR](T)[sP].[dR](G)[sP].[dR](T)[sP].[dR](G)
[sP].[dR](T)[sP].[LR]([5meC])[PS2].[MOE](T) (SEQ ID NO 5, CMP ID NO 5_3),
(s)
[MOE](T)[sP].[MOE](A)[sP].[MOE](G)[sP].[MOE](A)[sP].[LR](G)[PS2].[dR](T)[sP].[dR]
(T)[sP].[dR](T)[sP].[dR](T)[sP].[dR](G)[sP].[dR](T)[sP].[dR](G)[sP].[mR](U)[sP].[dR]
(G)[sP].[dR](T)[sP].[LR]([5meC])[PS2].[MOE](T) (SEQ ID NO 13, CMP ID NO 13_1),
(t)
[LR](A)[PS2].[LR](T)[sP].[LR](A)[sP].[LR](G)[sP].[dR](A)[sP].[dR]([PPG])[sP].[dR](T)
[sP].[dR](T)[sP].[dR](T)[sP].[dR](T)[sP].[dR](G)[sP].[dR](T)[sP].[dR](G)[sP].[dR](T)
[sP].[dR](G)[sP].[LR](T)[PS2].[LR]([5meC]) (SEQ ID NO 14, CMP ID NO 14_1),
(u)
[LR](A)[PS2].[LR](A)[sP].[LR](A)[sP].[dR](A)[sP].[dR]([PPG])[P].[dR](T)[sP].[dR](T)
[sP].[dR](T)[sP].[dR](C)[sP].[dR](T)[sP].[dR](C)[sP].[dR](A)[sP].[dR](G)[sP].[dR](A)
[sP].[dR](T)[sP].[dR](A)[sP].[LR](G)[P].[LR](G)[PS2].[LR]([5meC])
(SEQ ID NO 15, CMP ID NO 15_1),
(v)
[LR](A)[PS2].[LR](A)[sP].[LR](A)[sP].[dR](A)[sP].[dR](G)[sP].[dR](T)[sP].[dR](T)[sP].
[dR](T)[sP].[dR](C)[sP].[dR](T)[sP].[dR](C)[sP].[dR](A)[sP].[dR]([PPG])[sP].[dR](A)
[sP].[dR](T)[sP].[dR](A)[sP].[LR](G)[sP].[LR](G)[PS2].[LR]([5meC])
(SEQ ID NO 16, CMP ID NO 16_1),
(w)
[MOE](A)[sP].[LR](A)[PS2].[LR](A)[sP].[dR](A)[sP].[dR](G)[sP].[dR](T)[sP].[dR](T)[sP].
[dR](T)[sP].[dR](C)[sP].[dR](T)[sP].[dR](C)[sP].[dR](A)[sP].[dR]([PPG])[sP].[dR](A)
[sP].[dR](T)[sP].[dR](A)[sP].[LR](G)[sP].[LR](G)[PS2].[MOE]([5meC])
(SEQ ID NO 16, CMP ID NO 16_2),
(x)
[MOE](A)[sP].[LR](A)[PS2].[LR](A)[sP].[dR](A)[sP].[dR](G)[sP].[dR](T)[sP].[dR](T)[sP].
[dR](T)[sP].[dR](C)[sP].[dR](T)[sP].[dR](C)[sP].[dR](A)[sP].[dR]([PPG])[sP].[dR](A)
[sP].[dR](T)[sP].[dR](A)[sP].[LR](G)[PS2].[LR](G)[PS2].[MOE]([5meC])
(SEQ ID NO 16, CMP ID NO 16_3),
(y)
[LR](A)[PS2].[LR](A)[sP].[dR](A)[sP].[mR](A)[sP].[LR](G)[sP].[dR](T)[sP].[dR](T)[sP].
[dR](T)[sP].[dR](C)[sP].[dR](T)[sP].[dR](C)[sP].[dR](A)[sP].[dR](G)[sP].[dR](A)[sP].
[dR](T)[sP].[dR](A)[sP].[dR](G)[sP].[LR](G)[PS2].[LR]([5meC])
(SEQ ID NO 2, CMP ID NO 2_3),
(z)
[LR](A)[PS2].[LR](A)[PS2].[dR](A)[sP].[mR](A)[sP].[LR](G)[sP].[dR](T)[sP].[dR](T)[sP].
[dR](T)[sP].[dR](C)[sP].[dR](T)[sP].[dR](C)[sP].[dR](A)[sP].[dR](G)[sP].[dR](A)[sP].
[dR](T)[sP].[dR](A)[sP].[mR](G)[sP].[LR](G)[PS2].[LR]([5meC])
(SEQ ID NO 2, CMP ID NO 2_4),
(aa)
[LR](A)[PS2].[LR](A)[sP].[LR](A)[sP].[dR](A)[sP].[dR](G)[sP].[dR](T)[sP].[dR](T)[sP].
[dR](T)[sP].[dR](C)[sP].[dR](T)[sP].[dR](C)[sP].[mR](A)[sP].[dR](G)[sP].[dR](A)[sP].
[dR](T)[sP].[dR](A)[sP].[LR](G)[sP].[LR](G)[PS2].[LR]([5meC])
(SEQ ID NO 2, CMP ID NO 2_5),
(ab)
[LR](A)[PS2].[MOE](A)[sP].[MOE](A)[sP].[dR](A)[sP].[dR](G)[sP].[dR](T)[sP].[dR](T)
[sP].[dR](T)[sP].[dR](C)[sP].[dR](T)[sP].[dR](C)[sP].[mR](A)[sP].[dR](G)[sP].[dR](A)
[sP].[dR](T)[sP].[dR](A)[sP].[LR](G)[PS2].[LR](G)[PS2].[MOE]([5meC])
(SEQ ID NO 2, CMP ID NO 2_6),
(ac)
[LR](A)[PS2].[LR](A)[sP].[LR](A)[sP].[dR](A)[sP].[dR](G)[sP].[dR](T)[sP].[dR](T)[sP].
[dR](T)[sP].[dR](C)[sP].[dR](T)[sP].[mR](C)[sP].[dR](A)[sP].[dR](G)[sP].[dR](A)[sP].
[dR](T)[sP].[dR](A)[sP].[LR](G)[sP].[LR](G)[PS2].[LR]([5meC])
(SEQ ID NO 2, CMP ID NO 2_7),
(ad)
[MOE](A)[sP].[MOE](A)[sP].[LR](A)[PS2].[dR](A)[P].[dR](G)[sP].[dR](T)[sP].[dR](T)
[sP].[dR](T)[sP].[dR](C)[P].[dR](T)[sP].[mR](C)[sP].[dR](A)[P].[dR](G)[sP].[dR](A)
[sP].[dR](T)[sP].[dR](A)[P].[LR](G)[sP].[LR](G)[PS2].[MOE]([5meC])
(SEQ ID NO 2, CMP ID NO 2_8),
(ae)
[MOE](A)[sP].[LR](A)[sP].[LR](A)[sP].[dR](A)[sP].[dR](G)[sP].[dR](T)[sP].[dR](T)[sP].
[dR](T)[sP].[dR](C)[sP].[dR](T)[sP].[dR](C)[sP].[mR](A)[sP].[dR](G)[sP].[dR](A)[sP].
[dR](T)[sP].[dR](A)[sP].[LR](G)[sP].[LR](G)[P].[MOE]([5meC])
(SEQ ID NO 2, CMP ID NO 2_9),
(af)
[LR](A)[PS2].[LR](A)[sP].[LR](A)[sP].[dR](A)[sP].[dR](G)[sP].[dR](T)[sP].[dR](T)[sP].
[dR](T)[sP].[mR](C)[P].[dR](T)[sP].[dR](C)[sP].[dR](A)[sP].[dR](G)[sP].[dR](A)[sP].
[dR](T)[sP].[dR](A)[sP].[LR](G)[P].[LR](G)[PS2].[LR]([5meC])
(SEQ ID NO 2, CMP ID NO 2_10),
(ag)
[MOE](A)[sP].[LR](A)[sP].[LR](A)[sP].[dR](A)[sP].[dR](G)[P].[dR](T)[sP].[dR](T)[sP].
[dR](T)[sP].[mR](C)[sP].[dR](T)[sP].[dR](C)[sP].[dR](A)[sP].[dR](G)[sP].[dR](A)[sP].
[dR](T)[sP].[dR](A)[sP].[LR](G)[sP].[LR](G)[PS2].[MOE]([5meC])
(SEQ ID NO 2, CMP ID NO 2_11),
(ah)
[MOE](A)[sP].[LR](A)[PS2].[LR](A)[sP].[dR](A)[sP].[dR](G)[sP].[dR](T)[sP].[dR](T)[sP].
[dR](T)[sP].[dR](C)[sP].[dR](T)[sP].[dR](C)[sP].[dR](A)[sP].[dR](G)[sP].[dR](A)[sP].
[dR](T)[sP].[mR](A)[sP].[LR](G)[sP].[LR](G)[PS2].[MOE]([5meC])
(SEQ ID NO 2, CMP ID NO 2_12),
(ai)
[MOE](A)[sP].[MOE](A)[sP].[LR](A)[sP].[dR](A)[sP].[dR](G)[sP].[dR](T)[sP].[dR](T)
[sP].[mR](U)[sP].[dR](C)[sP].[dR](T)[sP].[dR](C)[sP].[dR](A)[sP].[dR](G)[sP].[dR](A)
[sP].[dR](T)[sP].[dR](A)[sP].[LR](G)[sP].[LR](G)[PS2].[MOE]([5meC])
(SEQ ID NO 17, CMP ID NO 17_1),
(aj)
[LR](A)[PS2].[LR](G)[sP].[dR](G)[sP].[dR](C)[sP].[LR](A)[sP].[dR](T)[sP].[dR](T)[sP].
[dR](A)[sP].[dR](T)[sP].[dR](A)[sP].[dR](G)[sP].[dR](G)[sP].[mR](U)[sP].[dR](T)[sP].
[dR](T)[sP].[LR](T)[sP].[LR](T)[PS2].[LR]([5meC])
(SEQ ID NO 18, CMP ID NO 18_1),
(ak)
[LR](A)[PS2].[LR](G)[sP].[dR](G)[sP].[dR](C)[sP].[LR](A)[sP].[mR](U)[sP].[dR](T)[sP].
[dR](A)[sP].[dR](T)[sP].[dR](A)[sP].[dR](G)[sP].[dR](G)[sP].[dR](T)[sP].[dR](T)[sP].
[dR](T)[sP].[LR](T)[sP].[LR](T)[PS2].[LR]([5meC])
(SEQ ID NO 19, CMP ID NO 19_1),
(al)
[LR](G)[PS2].[LR]([5meC])[sP].[dR](A)[sP].[dR](T)[sP].[dR](T)[sP].[dR](A)[sP].[dR](T)
[sP].[dR](A)[sP].[dR](G)[sP].[dR](G)[sP].[dR](T)[P].[LR](T)[sP].[dR](T)[sP].[dR](T)
[sP].[dR](T)[sP].[dR](C)[sP].[LR](T)[PS2].[LR]([5meC])
(SEQ ID NO 20, CMP ID NO 20_1),
(am)
[LR](G)[PS2].[LR]([5meC])[sP].[mR](A)[sP].[dR](T)[sP].[dR](T)[sP].[dR](A)[sP].[dR](T)
[P].[dR](A)[sP].[dR](G)[sP].[dR](G)[sP].[dR](T)[sP].[LR](T)[sP].[dR](T)[sP].[dR](T)
[sP].[dR](T)[sP].[dR](C)[sP].[LR](T)[PS2].[LR]([5meC])
(SEQ ID NO 20, CMP ID NO 20_2),
(an)
[LR](G)[PS2].[LR]([5meC])[sP].[dR](A)[sP].[dR](T)[sP].[dR](T)[sP].[dR](A)[sP].[dR](T)
[sP].[mR](A)[sP].[dR](G)[sP].[dR](G)[sP].[dR](T)[sP].[LR](T)[sP].[dR](T)[sP].[dR](T)
[sP].[dR](T)[sP].[dR](C)[sP].[LR](T)[PS2].[LR]([5meC])
(SEQ ID NO 20, CMP ID NO 20_3),
(ao)
[LR](G)[PS2].[LR]([5meC])[sP].[dR](A)[sP].[dR](T)[sP].[dR](T)[sP].[dR](A)[sP].[dR](T)
[sP].[dR](A)[sP].[mR](G)[sP].[dR](G)[sP].[dR](T)[sP].[LR](T)[sP].[dR](T)[sP].[dR](T)
[sP].[dR](T)[sP].[dR](C)[sP].[LR](T)[PS2].[LR]([5meC])
(SEQ ID NO 20, CMP ID NO 20_4),
(ap)
[LR](G)[PS2].[LR]([5meC])[sP].[dR](A)[sP].[dR](T)[sP].[dR](T)[sP].[dR](A)[sP].[dR](T)
[sP].[dR](A)[sP].[dR](G)[sP].[dR](G)[sP].[dR](T)[sP].[LR](T)[sP].[dR](T)[sP].[dR](T)
[sP].[dR](T)[sP].[dR](C)[sP].[LR](T)[PS2].[MOE]([5meC])
(SEQ ID NO 20, CMP ID NO 20_5),
(aq)
[LR](G)[PS2].[LR]([5meC])[sP].[dR](A)[sP].[dR](T)[sP].[dR](T)[sP].[dR](A)[sP].[dR](T)
[sP].[dR](A)[sP].[dR](G)[P].[mR](G)[sP].[dR](T)[sP].[LR](T)[sP].[dR](T)[sP].[dR](T)
[sP].[dR](T)[sP].[dR](C)[sP].[LR](T)[PS2].[LR]([5meC])
(SEQ ID NO 20, CMP ID NO 20_6),
(ar)
[LR](G)[PS2].[LR]([5meC])[sP].[LR](A)[sP].[dR](T)[sP].[dR](T)[sP].[dR](A)[sP].[dR](T)
[sP].[dR](A)[sP].[dR](G)[sP].[dR](G)[sP].[dR](T)[sP].[dR](T)[sP].[dR](T)[sP].[LR](T)
[sP].[dR](T)[sP].[LR]([5meC])[PS2].[LR](T) (SEQ ID NO 6, CMP ID NO 6_2),
(as)
[LR](G)[PS2].[LR]([5meC])[sP].[LR](A)[sP].[dR](T)[sP].[dR](T)[sP].[dR](A)[sP].[dR](T)
[sP].[mR](A)[sP].[dR](G)[sP].[dR](G)[sP].[dR](T)[sP].[dR](T)[sP].[dR](T)[sP].[LR](T)
[sP].[dR](T)[sP].[LR]([5meC])[PS2].[LR](T) (SEQ ID NO 6, CMP ID NO 6_3),
(at)
[LR](G)[PS2].[LR]([5meC])[sP].[LR](A)[sP].[dR](T)[sP].[dR](T)[sP].[dR](A)[sP].[dR](T)
[sP].[dR](A)[sP].[mR](G)[sP].[dR](G)[sP].[dR](T)[sP].[dR](T)[sP].[dR](T)[sP].[LR](T)
[sP].[dR](T)[P].[LR]([5meC])[PS2].[LR](T) (SEQ ID NO 6, CMP ID NO 6_4),
(au)
[LR](G)[PS2].[LR]([5meC])[sP].[mR](A)[sP].[dR](T)[sP].[dR](T)[sP].[dR](A)[sP].[dR](T)
[sP].[dR](A)[sP].[dR](G)[sP].[dR](G)[sP].[dR](T)[sP].[dR](T)[sP].[dR](T)[sP].[LR](T)
[sP].[LR](T)[sP].[LR]([5meC])[PS2].[LR](T) (SEQ ID NO 6, CMP ID NO 6_5),
(av)
[LR](G)[PS2].[LR]([5meC])[sP].[dR](A)[sP].[dR](T)[sP].[dR](T)[sP].[dR](A)[sP].[dR](T)
[sP].[mR](A)[sP].[dR](G)[sP].[dR](G)[sP].[dR](T)[sP].[dR](T)[sP].[dR](T)[sP].[LR](T)
[sP].[LR](T)[sP].[LR]([5meC])[PS2].[LR](T) (SEQ ID NO 6, CMP ID NO 6_6),
(aw)
[LR](G)[PS2].[LR]([5meC])[sP].[dR](A)[sP].[dR](T)[sP].[dR](T)[sP].[dR](A)[sP].[dR](T)
[sP].[dR](A)[sP].[mR](G)[sP].[dR](G)[sP].[dR](T)[sP].[dR](T)[sP].[dR](T)[sP].[LR](T)
[sP].[LR](T)[sP].[LR]([5meC])[PS2].[LR](T) (SEQ ID NO 6, CMP ID NO 6_7),
(ax)
[LR](G)[PS2].[LR]([5meC])[sP].[dR](A)[sP].[dR](T)[sP].[dR](T)[sP].[dR](A)[sP].[dR](T)
[sP].[dR](A)[sP].[dR](G)[sP].[mR](G)[sP].[dR](T)[P].[dR](T)[sP].[dR](T)[sP].[LR](T)
[sP].[LR](T)[sP].[LR]([5meC])[PS2].[LR](T) (SEQ ID NO 6, CMP ID NO 6_8),
(ay)
[MOE](G)[sP].[LR]([5meC])[PS2].[dR](A)[sP].[dR](T)[sP].[dR](T)[sP].[dR](A)[sP].[dR]
(T)[sP].[dR](A)[sP].[dR](G)[sP].[dR](G)[sP].[dR](T)[sP].[dR](T)[sP].[dR](T)[sP].[LR](T)
[sP].[LR](T)[P].[LR]([5meC])[PS2].[MOE](T) (SEQ ID NO 6, CMP ID NO 6_9),
(az)
[MOE](G)[sP].[LR]([5meC])[PS2].[dR](A)[sP].[dR](T)[sP].[dR](T)[sP].[dR](A)[sP].[dR]
(T)[sP].[dR](A)[sP].[dR](G)[sP].[dR](G)[sP].[dR](T)[sP].[dR](T)[sP].[dR](T)[sP].[LR](T)
[sP].[LR](T)[PS2].[MOE]([5meC])[sP].[MOE](T) (SEQ ID NO 6, CMP ID NO 6_10),
(ba)
[MOE](G)[sP].[LR]([5meC])[PS2].[dR](A)[sP].[dR](T)[sP].[dR](T)[sP].[dR](A)[sP].[dR]
(T)[sP].[dR](A)[sP].[dR](G)[sP].[dR](G)[sP].[dR](T)[sP].[dR](T)[sP].[dR](T)[sP].[LR](T)
[sP].[LR](T)[PS2].[LR]([5meC])[PS2].[MOE](T) (SEQ ID NO 6, CMP ID NO 6_11),
(bb)
[MOE](G)[sP].[LR]([5meC])[PS2].[dR](A)[sP].[dR](T)[sP].[dR](T)[sP].[dR](A)[sP].[dR]
(T)[sP].[dR](A)[sP].[dR](G)[sP].[dR](G)[P].[dR](T)[sP].[dR](T)[sP].[dR](T)[sP].[LR](T)
[PS2].[LR](T)[PS2].[MOE]([5meC])[sP].[MOE](T) (SEQ ID NO 6, CMP ID NO 6_12),
(bc)
[LR](G)[PS2].[LR]([5meC])[sP].[dR](A)[sP].[dR](T)[sP].[dR](T)[sP].[dR](A)[sP].[dR](T)
[sP].[dR](A)[sP].[dR](G)[sP].[dR](G)[sP].[dR](T)[sP].[LR](T)[sP].[dR](T)[sP].[dR](T)
[sP].[mR](U)[sP].[dR](C)[sP].[LR](T)[PS2].[LR]([5meC])
(SEQ ID NO 21, CMP ID NO 21_1),
(bd)
[LR](G)[PS2].[LR]([5meC])[sP].[dR](A)[P].[dR](T)[sP].[dR](T)[sP].[dR](A)[sP].[dR](T)
[sP].[dR](A)[sP].[dR](G)[sP].[dR](G)[sP].[dR](T)[sP].[LR](T)[sP].[dR](T)[sP].[mR](U)
[sP].[dR](T)[sP].[dR](C)[sP].[LR](T)[PS2].[LR]([5meC])
(SEQ ID NO 22, CMP ID NO 22_1),
(be)
[LR](G)[PS2].[LR]([5meC])[sP].[dR](A)[sP].[dR](T)[sP].[dR](T)[sP].[dR](A)[sP].[dR](T)
[sP].[dR](A)[sP].[dR](G)[sP].[dR](G)[sP].[dR](T)[sP].[LR](T)[sP].[mR](U)[sP].[dR](T)
[sP].[dR](T)[sP].[dR](C)[sP].[LR](T)[PS2].[LR]([5meC])
(SEQ ID NO 23, CMP ID NO 23_1),
(bf)
[LR](G)[PS2].[LR]([5meC])[sP].[dR](A)[sP].[dR](T)[sP].[dR](T)[sP].[dR](A)[sP].[dR](T)
[sP].[dR](A)[sP].[dR](G)[sP].[dR](G)[sP].[dR](T)[sP].[dR](T)[sP].[mR](U)[sP].[LR](T)
[sP].[LR](T)[sP].[LR]([5meC])[PS2].[LR](T) (SEQ ID NO 24, CMP ID NO 24_1),
(bg)
[LR](G)[PS2].[LR]([5meC])[sP].[LR](A)[sP].[dR](T)[sP].[dR](T)[sP].[dR](A)[sP].[dR](T)
[sP].[dR](A)[sP].[dR](G)[sP].[dR](G)[sP].[dR](T)[sP].[mR](U)[sP].[dR](T)[sP].[LR](T)
[sP].[dR](T)[sP].[LR]([5meC])[PS2].[LR](T) (SEQ ID NO 25, CMP ID NO 25_1),
(bh)
[LR](G)[PS2].[LR]([5meC])[sP].[dR](A)[sP].[dR](T)[sP].[dR](T)[sP].[dR](A)[sP].[dR](T)
[sP].[dR](A)[sP].[dR](G)[sP].[dR](G)[sP].[dR](T)[sP].[mR](U)[sP].[dR](T)[sP].[LR](T)
[sP].[LR](T)[sP].[LR]([5meC])[PS2].[LR](T) (SEQ ID NO 25, CMP ID NO 25_2),
(bi)
[LR](G)[PS2].[LR]([5meC])[sP].[dR](A)[P].[dR](T)[P].[dR](T)[sP].[dR](A)[sP].[dR](T)
[sP].[dR](A)[sP].[dR](G)[sP].[dR](G)[sP].[mR](U)[sP].[LR](T)[sP].[dR](T)[sP].[dR](T)
[sP].[dR](T)[sP].[dR](C)[sP].[LR](T)[PS2].[LR]([5meC])
(SEQ ID NO 26, CMP ID NO 26_1),
(bj)
[LR](G)[PS2].[LR]([5meC])[P].[LR](A)[sP].[dR](T)[sP].[dR](T)[sP].[dR](A)[sP].[dR](T)
[sP].[dR](A)[sP].[dR](G)[sP].[dR](G)[sP].[mR](U)[sP].[dR](T)[sP].[dR](T)[sP].[LR](T)
[sP].[dR](T)[sP].[LR]([5meC])[PS2].[LR](T) (SEQ ID NO 27, CMP ID NO 27_1),
(bk)
[LR](G)[PS2].[LR]([5meC])[sP].[dR](A)[sP].[dR](T)[sP].[dR](T)[sP].[dR](A)[sP].[dR](T)
[sP].[dR](A)[sP].[dR](G)[sP].[dR](G)[sP].[mR](U)[sP].[dR](T)[sP].[dR](T)[sP].[LR](T)
[sP].[LR](T)[sP].[LR]([5meC])[PS2].[LR](T) (SEQ ID NO 27, CMP ID NO 27_2),
(bl)
[LR](G)[PS2].[LR]([5meC])[sP].[dR](A)[sP].[dR](T)[sP].[dR](T)[sP].[dR](A)[sP].[mR]
(U)[sP].[dR](A)[sP].[dR](G)[sP].[dR](G)[sP].[dR](T)[sP].[dR](T)[sP].[dR](T)[sP].[LR](T)
[sP].[LR](T)[sP].[LR]([5meC])[PS2].[LR](T) (SEQ ID NO 28, CMP ID NO 28_1),
(bm)
[LR](G)[PS2].[LR]([5meC])[sP].[dR](A)[sP].[dR](T)[sP].[mR](U)[sP].[dR](A)[sP].[dR]
(T)[sP].[dR](A)[sP].[dR](G)[sP].[dR](G)[sP].[dR](T)[sP].[dR](T)[sP].[dR](T)[sP].[LR](T)
[sP].[LR](T)[sP].[LR]([5meC])[PS2].[LR](T) (SEQ ID NO 29, CMP ID NO 29_1),
(bn)
[LR](G)[PS2].[LR]([5meC])[sP].[dR](A)[sP].[mR](U)[sP].[dR](T)[sP].[dR](A)[sP].[dR]
(T)[sP].[dR](A)[sP].[dR](G)[sP].[dR](G)[sP].[dR](T)[sP].[dR](T)[sP].[dR](T)[sP].[LR](T)
[sP].[LR](T)[sP].[LR]([5meC])[PS2].[LR](T) (SEQ ID NO 30, CMP ID NO 30_1),
(bo)
[LR](G)[sP].[LR](T)[sP].[LR](T)[sP].[dR](T)[sP].[dR](T)[sP].[dR](A)[sP].[dR](A)[sP].
[mR](G)[sP].[dR](A)[sP].[dR](T)[sP].[dR](T)[sP].[dR](G)[sP].[dR](T)[sP].[dR](G)[sP].
[dR](G)[sP].[dR](T)[sP].[dR](G)[sP].[LR](T)[sP].[LR](T)[sP].[LR](T)
(SEQ ID NO 11, CMP ID NO 11_2), and
(bp)
[LR](G)[sP].[LR](T)[sP].[LR](T)[sP].[dR](T)[sP].[dR](T)[sP].[dR](A)[sP].[dR](A)[sP].
[mR](G)[sP].[dR](A)[sP].[dR](T)[sP].[dR](T)[sP].[dR](G)[sP].[dR](T)[sP].[dR](G)[sP].
[dR](G)[sP].[dR](T)[sP].[dR](G)[sP].[LR](T)[sP].[LR](T)[PS2].[LR](T)
(SEQ ID NO 11, CMP ID NO 11_3),

wherein
(A) is a nucleoside comprising an adenine nucleobase,
(C) is a nucleoside comprising a cytosine nucleobase,
([5meC]) is a nucleoside comprising a 5-methyl cytosine nucleobase,
([PPG]) is a nucleoside comprising a 7-deaza-8-aza guanine nucleobase,
(G) is a nucleoside comprising a guanine nucleobase,
(U) is a nucleoside comprising a uracil nucleobase,
(T) is a nucleoside comprising a thymine nucleobase,
[LR] indicates that the following nucleoside is a beta-D-oxy-LNA nucleoside,
[dR] indicates that the following nucleoside is a DNA nucleoside,
[mR] indicates that the following nucleoside is a 2′-O-methyl RNA nucleoside,
[MOE] indicates that the following nucleoside is a 2′-O-methoxyethyl RNA (MOE RNA) nucleoside,
[sP] indicates a phosphorothioate internucleoside linkage between the preceding and following nucleoside, and
[PS2] indicates a phosphorodithioate internucleoside linkage between the preceding and following nucleoside.

The invention also provides an antisense oligonucleotide comprising or consisting of a compound selected from the following:

(a)
[LR](A)[sP].[LR](G)[sP].[dR](G)[sP].[dR](C)[P].[LR](A)[sP].[dR](T)[sP].[dR](T)[sP].
[dR](A)[sP].[dR](T)[sP].[dR](A)[sP].[dR](G)[sP].[dR](G)[sP].[dR](T)[sP].[dR](T)[sP].
[dR](T)[sP].[LR](T)[sP].[LR](T)[sP].[LR]([5meC]) (SEQ ID NO 1, CMP ID NO 1_1),
(b)
[LR](A)[PS2].[LR](A)[sP].[dR](A)[sP].[mR](A)[sP].[LR](G)[sP].[dR](T)[sP].[dR](T)[sP].
[dR](T)[sP].[dR](C)[sP].[dR](T)[sP].[dR](C)[sP].[dR](A)[sP].[dR](G)[sP].[dR](A)[sP].
[dR](T)[sP].[dR](A)[sP].[mR](G)[sP].[LR](G)[PS2].[LR]([5meC])
(SEQ ID NO 2, CMP ID NO 2_1),
(c)
[LR](G)[sP].[LR](G)[sP].[mR](A)[sP].[dR](C)[sP].[dR](A)[sP].[dR](T)[sP].[LR](G)[sP].
[dR](T)[sP].[dR](T)[sP].[dR](A)[sP].[dR](A)[sP].[dR](T)[sP].[dR](T)[sP].[dR](T)[sP].
[dR](T)[sP].[dR](A)[sP].[dR](C)[sP].[LR](T)[PS2].[LR](T) (SEQ ID NO 3, CMP ID NO 3_1),
(d)
[LR](T)[PS2].[LR](G)[sP].[LR](A)[sP].[dR](A)[sP].[dR](C)[sP].[dR](T)[sP].[dR](T)[sP].
[dR](G)[sP].[dR](T)[sP].[dR](T)[sP].[dR](A)[sP].[dR](T)[sP].[dR](A)[sP].[dR](T)[sP].
[dR](A)[sP].[dR](C)[sP].[LR]([5meC])[PS2].[LR](T)[PS2].[LR](G)
(SEQ ID NO 4, CMP ID NO 4_1),
(e)
[LR](T)[PS2].[LR](G)[sP].[LR](A)[sP].[mR](A)[sP].[dR](C)[sP].[dR](T)[sP].[dR](T)[sP].
[dR](G)[sP].[dR](T)[sP].[dR](T)[sP].[dR](A)[sP].[dR](T)[sP].[dR](A)[sP].[dR](T)[sP].
[dR](A)[sP].[dR](C)[sP].[LR]([5meC])[sP].[LR](T)[PS2].[LR](G)
(SEQ ID NO 4, CMP ID NO 4_2),
(f)
[LR](T)[PS2].[LR](A)[PS2].[dR](G)[sP].[dR](A)[sP].[LR](G)[sP].[dR](T)[sP].[dR](T)[sP].
[dR](T)[sP].[dR](T)[sP].[dR](G)[sP].[dR](T)[sP].[dR](G)[sP].[dR](T)[sP].[dR](G)[sP].
[dR](T)[sP].[MOE]([5meC])[sP].[MOE](T) (SEQ ID NO 5, CMP ID NO 5_1),
(g)
[LR](T)[PS2].[LR](A)[sP].[dR](G)[sP].[dR](A)[sP].[LR](G)[sP].[dR](T)[sP].[dR](T)[sP].
[dR](T)[sP].[dR](T)[sP].[dR](G)[sP].[dR](T)[sP].[dR](G)[sP].[dR](T)[P].[dR](G)[sP].
[dR](T)[sP].[MOE]([5meC])[sP].[MOE](T) (SEQ ID NO 5, CMP ID NO 5_2),
(h)
[LR](G)[sP].[LR]([5meC])[sP].[dR](A)[sP].[dR](T)[sP].[dR](T)[sP].[dR](A)[sP].[dR](T)
[sP].[dR](A)[sP].[dR](G)[sP].[dR](G)[sP].[dR](T)[sP].[dR](T)[sP].[dR](T)[sP].[LR](T)
[sP].[LR](T)[sP].[LR]([5meC])[sP].[LR](T) (SEQ ID NO 6, CMP ID NO 6_1),
(i)
[MOE](G)[sP].[LR](G)[sP].[dR](C)[sP].[LR](A)[sP].[dR](T)[sP].[dR](T)[sP].[dR](A)[sP].
[dR](T)[sP].[dR](A)[sP].[dR](G)[sP].[dR](G)[sP].[dR](T)[sP].[dR](T)[sP].[LR](T)[sP].
[MOE](T)[sP].[MOE](T)[sP].[MOE]([5meC]) (SEQ ID NO 7, CMP ID NO 7_1),
(j)
[LR](G)[sP].[LR](G)[sP].[dR](C)[sP].[LR](A)[sP].[dR](T)[sP].[dR](T)[sP].[dR](A)[sP].
[dR](T)[sP].[mR](A)[sP].[dR](G)[sP].[dR](G)[sP].[dR](T)[sP].[dR](T)[sP].[dR](T)[sP].
[LR](T)[sP].[LR](T)[sP].[LR]([5meC]) (SEQ ID NO 7, CMP ID NO 7_2),
(k)
[MOE](G)[sP].[MOE](G)[sP].[MOE]([5meC])[sP].[LR](A)[PS2].[dR](T)[sP].[dR](T)[sP].
[dR](A)[sP].[dR](T)[sP].[mR](A)[sP].[dR](G)[sP].[dR](G)[sP].[dR](T)[sP].[dR](T)[sP].
[LR](T)[PS2].[LR](T)[PS2].[LR](T)[PS2].[MOE]([5meC])
(SEQ ID NO 8, CMP ID NO 8_1),
(l)
[LR](A)[PS2].[LR](A)[PS2].[dR](A)[sP].[mR](A)[sP].[LR](G)[sP].[dR](T)[sP].[dR](T)[sP].
[dR](T)[sP].[dR](C)[sP].[dR](T)[sP].[dR](C)[sP].[dR](A)[sP].[dR](G)[sP].[dR](A)[sP].
[dR](T)[sP].[dR](A)[sP].[dR](G)[sP].[LR](G)[PS2].[LR]([5meC])
(SEQ ID NO 2, CMP ID NO 2_2),
(m)
[LR](T)[PS2].[LR](G)[sP].[dR](G)[sP].[LR](A)[sP].[dR](C)[sP].[dR](A)[sP].[mR](U)[sP].
[dR](G)[sP].[dR](T)[sP].[LR](T)[sP].[dR](A)[sP].[dR](A)[sP].[dR](T)[sP].[dR](T)[sP].
[dR](T)[sP].[dR](T)[sP].[LR](A)[PS2].[LR]([5meC]) (SEQ ID NO 9, CMP ID NO 9_1),
(n)
[LR](G)[PS2].[LR](G)[PS2].[dR](A)[sP].[dR](C)[sP].[dR](A)[sP].[dR](T)[sP].[LR](G)[sP].
[mR](U)[sP].[dR](T)[sP].[mR](A)[sP].[dR](A)[sP].[dR](T)[sP].[dR](T)[sP].[dR](T)[sP].
[dR](T)[sP].[dR](A)[P].[dR](C)[sP].[LR](T)[PS2].[LR](T)
(SEQ ID NO 10, CMP ID NO 10_1),
(o)
[LR](G)[sP].[LR](T)[sP].[LR](T)[sP].[dR](T)[sP].[dR](T)[sP].[mR](A)[sP].[dR](A)[sP].
[dR](G)[sP].[dR](A)[sP].[dR](T)[sP].[dR](T)[sP].[dR](G)[sP].[dR](T)[sP].[dR](G)[sP].
[dR](G)[sP].[dR](T)[sP].[dR](G)[sP].[LR](T)[sP].[LR](T)[PS2].[LR](T)
(SEQ ID NO 11, CMP ID NO 11_1),
(p)
[LR](T)[PS2].[LR](G)[sP].[LR](A)[sP].[dR](A)[sP].[dR](C)[sP].[dR](T)[sP].[dR](T)[sP].
[dR](G)[P].[dR](T)[sP].[dR](T)[sP].[dR](A)[sP].[dR](T)[sP].[dR](A)[sP].[dR](T)[sP].
[dR](A)[sP].[dR](C)[sP].[LR]([5meC])[sP].[LR](T)[PS2].[LR](G)
(SEQ ID NO 4, CMP ID NO 4_3),
(q)
[LR](T)[PS2].[LR](A)[PS2].[dR](G)[sP].[dR](A)[sP].[LR](G)[sP].[dR](T)[sP].[dR](T)
[sP].[dR](T)[sP].[mR](U)[sP].[dR](G)[sP].[dR](T)[sP].[dR](G)[sP].[dR](T)[sP].[dR](G)
[sP].[dR](T)[sP].[MOE]([5meC])[sP].[MOE](T) (SEQ ID NO 12, CMP ID NO 12_1),
(r)
[MOE](T)[sP].[MOE](A)[sP].[MOE](G)[sP].[MOE](A)[sP].[LR](G)[PS2].[dR](T)[sP].[dR]
(T)[sP].[dR](T)[sP].[dR](T)[sP].[dR](G)[sP].[dR](T)[sP].[dR](G)[sP].[dR](T)[sP].[dR]
(G)[sP].[dR](T)[sP].[LR]([5meC])[PS2].[MOE](T) (SEQ ID NO 5, CMP ID NO 5_3),
(s)
[MOE](T)[sP].[MOE](A)[sP].[MOE](G)[sP].[MOE](A)[sP].[LR](G)[PS2].[dR](T)[sP].[dR]
(T)[sP].[dR](T)[sP].[dR](T)[sP].[dR](G)[sP].[dR](T)[sP].[dR](G)[sP].[mR](U)[sP].[dR]
(G)[sP].[dR](T)[sP].[LR]([5meC])[PS2].[MOE](T)
(SEQ ID NO 13, CMP ID NO 13_1),
and
(t)
[LR](A)[PS2].[LR](T)[sP].[LR](A)[sP].[LR](G)[sP].[dR](A)[sP].[dR]([PPG])[sP].[dR](T)
[sP].[dR](T)[sP].[dR](T)[sP].[dR](T)[sP].[dR](G)[sP].[dR](T)[P].[dR](G)[sP].[dR](T)
[sP].[dR](G)[sP].[LR](T)[PS2].[LR]([5meC]) (SEQ ID NO 14, CMP ID NO 14_1),

wherein
(A) is a nucleoside comprising an adenine nucleobase,
(C) is a nucleoside comprising a cytosine nucleobase,
([5meC]) is a nucleoside comprising a 5-methyl cytosine nucleobase,
([PPG]) is a nucleoside comprising a 7-deaza-8-aza guanine nucleobase,
(G) is a nucleoside comprising a guanine nucleobase,
(U) is a nucleoside comprising a uracil nucleobase,
(T) is a nucleoside comprising a thymine nucleobase,
[LR] indicates that the following nucleoside is a beta-D-oxy-LNA nucleoside,
[dR] indicates that the following nucleoside is a DNA nucleoside,
[mR] indicates that the following nucleoside is a 2′-O-methyl RNA nucleoside,
[MOE] indicates that the following nucleoside is a 2′-O-methoxyethyl RNA (MOE RNA) nucleoside,
[sP] indicates a phosphorothioate internucleoside linkage between the preceding and following nucleoside, and
[PS2] indicates a phosphorodithioate internucleoside linkage between the preceding and following nucleoside.

The invention also provides an antisense oligonucleotide comprising or consisting of a compound selected from the following:

(a)
[LR](A)[sP].[LR](G)[sP].[dR](G)[sP].[dR](C)[sP].[LR](A)[sP].[dR](T)[sP].[dR](T)[sP].
[dR](A)[sP].[dR](T)[sP].[dR](A)[sP].[dR](G)[sP].[dR](G)[P].[dR](T)[sP].[dR](T)[sP].
[dR](T)[sP].[LR](T)[sP].[LR](T)[sP].[LR]([5meC]) (SEQ ID NO 1, CMP ID NO 1_1),
(b)
[LR](A)[PS2].[LR](A)[sP].[dR](A)[sP].[mR](A)[sP].[LR](G)[sP].[dR](T)[sP].[dR](T)[sP].
[dR](T)[sP].[dR](C)[sP].[dR](T)[sP].[dR](C)[sP].[dR](A)[sP].[dR](G)[sP].[dR](A)[sP].
[dR](T)[sP].[dR](A)[sP].[mR](G)[sP].[LR](G)[PS2].[LR]([5meC])
(SEQ ID NO 2, CMP ID NO 2_1),
(c)
[LR](G)[P].[LR](G)[sP].[mR](A)[sP].[dR](C)[sP].[dR](A)[sP].[dR](T)[sP].[LR](G)[sP].
[dR](T)[sP].[dR](T)[sP].[dR](A)[sP].[dR](A)[sP].[dR](T)[sP].[dR](T)[sP].[dR](T)[sP].
[dR](T)[sP].[dR](A)[sP].[dR](C)[sP].[LR](T)[PS2].[LR](T) (SEQ ID NO 3, CMP ID NO 3_1),
(d)
[LR](T)[PS2].[LR](G)[sP].[LR](A)[sP].[dR](A)[sP].[dR](C)[sP].[dR](T)[sP].[dR](T)[sP].
[dR](G)[sP].[dR](T)[sP].[dR](T)[sP].[dR](A)[sP].[dR](T)[sP].[dR](A)[sP].[dR](T)[sP].
[dR](A)[sP].[dR](C)[sP].[LR]([5meC])[PS2].[LR](T)[PS2].[LR](G)
(SEQ ID NO 4, CMP ID NO 4_1),
(e)
[LR](T)[PS2].[LR](G)[sP].[LR](A)[sP].[mR](A)[sP].[dR](C)[sP].[dR](T)[sP].[dR](T)[sP].
[dR](G)[sP].[dR](T)[sP].[dR](T)[sP].[dR](A)[sP].[dR](T)[sP].[dR](A)[sP].[dR](T)[sP].
[dR](A)[sP].[dR](C)[sP].[LR]([5meC])[sP].[LR](T)[PS2].[LR](G)
(SEQ ID NO 4, CMP ID NO 4_2),
(f)
[LR](T)[PS2].[LR](A)[PS2].[dR](G)[sP].[dR](A)[sP].[LR](G)[sP].[dR](T)[sP].[dR](T)[sP].
[dR](T)[sP].[dR](T)[sP].[dR](G)[sP].[dR](T)[sP].[dR](G)[sP].[dR](T)[sP].[dR](G)[sP].
[dR](T)[sP].[MOE]([5meC])[sP].[MOE](T) (SEQ ID NO 5, CMP ID NO 5_1),
and
(g)
[LR](T)[PS2].[LR](A)[sP].[dR](G)[sP].[dR](A)[sP].[LR](G)[sP].[dR](T)[sP].[dR](T)[sP].
[dR](T)[sP].[dR](T)[sP].[dR](G)[sP].[dR](T)[sP].[dR](G)[sP].[dR](T)[sP].[dR](G)[sP].
[dR](T)[sP].[MOE]([5meC])[sP].[MOE](T) (SEQ ID NO 5, CMP ID NO 5_2),

wherein
(A) is a nucleoside comprising an adenine nucleobase,
(C) is a nucleoside comprising a cytosine nucleobase,
([5meC]) is a nucleoside comprising a 5-methyl cytosine nucleobase,
([PPG]) is a nucleoside comprising a 7-deaza-8-aza guanine nucleobase,
(G) is a nucleoside comprising a guanine nucleobase,
(U) is a nucleoside comprising a uracil nucleobase,
(T) is a nucleoside comprising a thymine nucleobase,
[LR] indicates that the following nucleoside is a beta-D-oxy-LNA nucleoside,
[dR] indicates that the following nucleoside is a DNA nucleoside,
[mR] indicates that the following nucleoside is a 2′-O-methyl RNA nucleoside,
[MOE] indicates that the following nucleoside is a 2′-O-methoxyethyl RNA (MOE RNA) nucleoside,
[sP] indicates a phosphorothioate internucleoside linkage between the preceding and following nucleoside, and
[PS2] indicates a phosphorodithioate internucleoside linkage between the preceding and following nucleoside.

The invention also provides an antisense oligonucleotide selected from the antisense oligonucleotides depicted by HELM strings in Table 4 herein.

Conjugates

The invention provides an antisense oligonucleotide conjugate comprising the antisense oligonucleotide of the invention covalently attached to at least one conjugate moiety. In other words, the invention provides an antisense oligonucleotide covalently attached to at least one conjugate moiety.

The term “antisense oligonucleotide conjugate” is used interchangeably herein with the terms “conjugate” and “conjugate of the invention”. The term “conjugate moiety” refers to a non-nucleotide moiety which can be covalently attached to an antisense oligonucleotide of the invention. Thus, the term “conjugate” as used herein refers to an antisense oligonucleotide of the invention which is covalently attached to a non-nucleotide moiety (conjugate moiety).

Oligonucleotide conjugates and their synthesis has also been reported in comprehensive reviews by Manoharan in Antisense Drug Technology, Principles, Strategies, and Applications, S. T. Crooke, ed., Ch. 16, Marcel Dekker, Inc., 2001 and Manoharan, Antisense and Nucleic Acid Drug Development, 2002, 12, 103.

In some embodiments, the at least one conjugate moiety is covalently attached to the 5′ end of the antisense oligonucleotide.

Conjugate Moieties

In some embodiments, the conjugate moiety (i.e. non-nucleotide moiety) is selected from the group consisting of carbohydrates (e.g. GalNAc), cell surface receptor ligands, drug substances, hormones, lipophilic substances, polymers, proteins, peptides, toxins (e.g. bacterial toxins), vitamins, viral proteins (e.g. capsids) or combinations thereof.

In some embodiments, the conjugate moiety is capable of binding to the asialoglycoprotein receptor, such as the human asialoglycoprotein receptor (ASGPR). For example, the conjugate moiety may comprise at least one asialoglycoprotein receptor targeting moiety selected from the group consisting of galactose, galactosamine, N-formyl-galactosamine, N-acetylgalactosamine, N-propionyl-galactosamine, N-n-butanoyl-galactosamine and N-isobutanoylgalactosamine.

In some embodiments, the asialoglycoprotein receptor-targeting moiety is N-acetylgalactosamine (GalNAc). In some embodiments, the conjugate moiety is an N-acetylgalactosamine (GalNAc) conjugate moiety. Thus, the antisense oligonucleotide of the present invention may be conjugated to at least one conjugate moiety comprising at least one N-acetylgalactosamine (GalNAc) moiety, such as at least one conjugate moiety comprising at least one N-acetylgalactosamine (GalNAc) moiety as described below.

In some embodiments, the conjugate moiety is an at least divalent, such as a divalent, trivalent or tetravalent, GalNAc. In preferred embodiments, the conjugate moiety is a trivalent GalNAc. Tri-valent N-acetylgalactosamine conjugate moieties are suitable for binding to the ASGPR, see for example WO 2014/076196, WO 2014/207232 and WO 2014/179620. Such conjugate moieties serve to enhance uptake of the oligonucleotide to the liver. The term “trivalent GalNAc” as used herein refers to a residue comprising three N-acetylgalactosamine moieties, i.e. preferably three moieties of formula

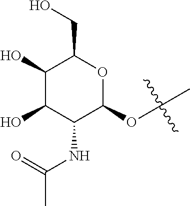 [SMILES: *O[C@@H]1O[C@H](CO)[C@H](O)[C@H](O)[C@H]1NC(C)=O]

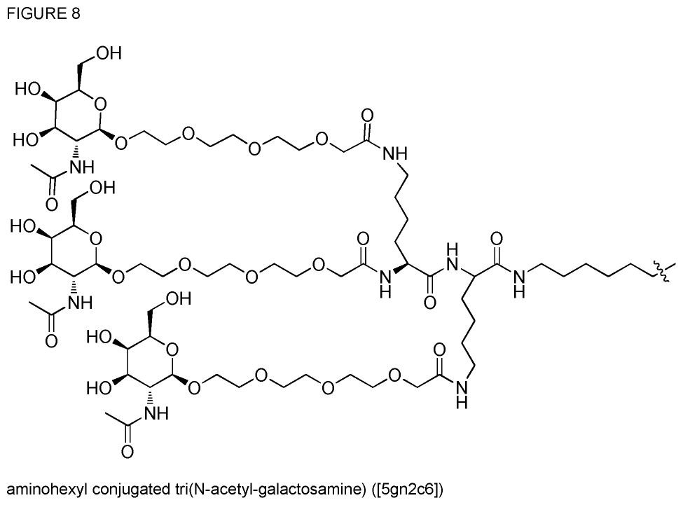

In some embodiments, the GalNAc conjugate moiety is aminohexyl conjugated tri(N-acetyl-galactosamine) as depicted below and in FIG. 8.

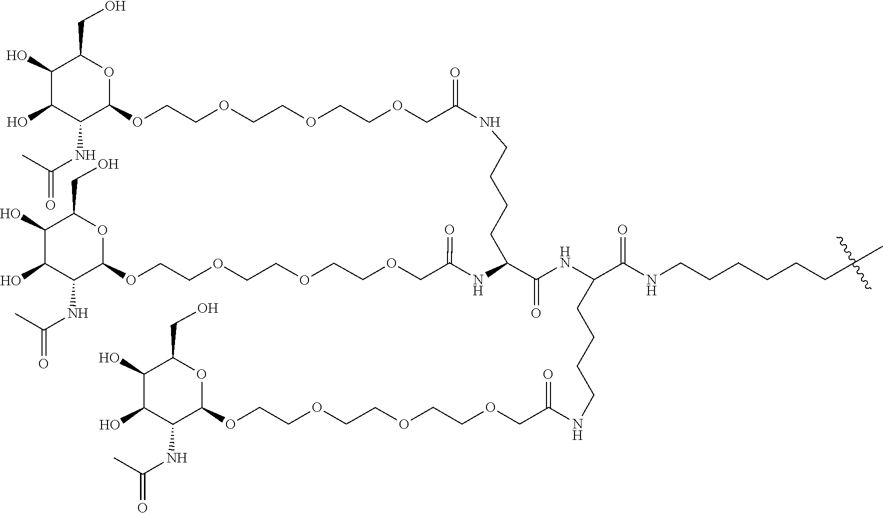 [SMILES: *CCCCCCNC(=O)C(CCCCNC(=O)COCCOCCOCCO[C@@H]1O[C@H](CO)[C@H](O)[C@H](O)[C@H]1NC(C)=O)NC(=O)[C@H](CCCCNC(=O)COCCOCCOCCO[C@@H]1O[C@H](CO)[C@H](O)[C@H](O)[C@H]1NC(C)=O)NC(=O)COCCOCCOCCO[C@@H]1O[C@H](CO)[C@H](O)[C@H](O)[C@H]1NC(C)=O]

The trivalent N-acetylgalactosamine (GalNAc) of FIG. 8 (and shown above) is also referred to herein as “5gn2c6”.

“Aminohexyl conjugated tri(N-acetyl-galactosamine)” may also be referred to as “-hexylene-NH-tri(N-acetyl-galactosamine)”.

In some embodiments, the conjugate moiety is covalently attached to the antisense oligonucleotide via a phosphodiester bond. In some embodiments, the conjugate moiety is covalently attached to the linker via a phosphodiester bond. In some embodiments, the GalNAc conjugate moiety as depicted in FIG. 8 is covalently attached to the antisense oligonucleotide via a phosphodiester bond. In some embodiments, the GalNAc conjugate moiety as depicted in FIG. 8 is covalently attached to the linker via a phosphodiester bond.

Linkers

In some embodiments of the antisense oligonucleotide conjugate of the invention, the conjugate moiety is covalently attached to the antisense oligonucleotide via a linker. Thus, in some embodiments, the conjugate comprises a linker. In some embodiments, the conjugate comprises a linker which is positioned between the antisense oligonucleotide and the conjugate moiety.

In some embodiments of the antisense oligonucleotide conjugate of the invention, the conjugate moiety is covalently attached to the antisense oligonucleotide via a linker nucleoside sequence. The linker nucleoside sequence may be referred to herein as region D′ or region D″.

In some embodiments, the linker nucleoside sequence comprises or consists of 1 to 10 linked nucleosides, such as 1, 2, 3, 4, 5, 6, 7, 8, 9 or 10 linked nucleosides, such as between 2 and 6 linked nucleosides, such as between 2 and 5 linked nucleosides, such as between 2 and 4 linked nucleosides. In some embodiments, the linker nucleoside sequence is 2 nucleosides in length. In some embodiments, the linker comprises two linked nucleosides. In some embodiments, the linker consists of two linked nucleosides.

In some embodiments, the linker nucleoside sequence comprises DNA nucleosides. In some embodiments, the linker nucleoside sequence consists of DNA nucleosides. In other words, in some embodiments, all nucleosides in the linker nucleoside sequence are DNA nucleosides.

In some embodiments, the nucleosides of the linker nucleoside sequence are linked via phosphodiester internucleoside linkages. In some embodiments, the linker is linked to the antisense oligonucleotide via a phosphodiester internucleoside linkage. In some embodiments, the linker is linked to the conjugate moiety via a phoisphodiester bond.

The terms “phosphodiester internucleoside linkage” and “phosphodiester bond” as used herein refer to the same chemical structure, known in the art, wherein a first chemical entity is linked to a second chemical entity via an intermediate phosphate group. The term “phosphodiester internucleoside linkage” is used particularly wherein the first chemical entity and second chemical entity that are linked are nucleosides, such as nucleosides of the antisense oligonucleotide of the invention. As is known in the art, the phosphodiester internucleoside linkage is the naturally occurring internucleoside in naturally occurring nucleic acids such as genomic DNA. The term “phosphodiester bond” is used particularly in the context of the present invention to refer to the covalent attachment of a conjugate moiety to the antisense oligonucleotide of the invention or to a linker, such as a nucleoside linker sequence, because the conjugate moiety is not a nucleoside so the term “phosphodiester internucleoside linkage” is not appriopriate. A phosphodiester internucleoside linkage/phosphodiester bond has the following structure

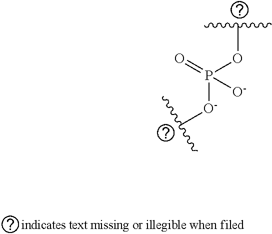

or in protonated form

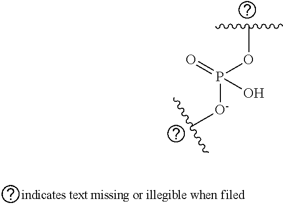

wherein the first chemical entity and second chemical entity are attached at positions 1 and 2 respectively. In some embodiments, the conjugate moiety is attached at position 1 and an antisense oligonucleotide of the invention is attached at position 2. In some embodiments, the conjugate moiety is attached at position 1 and a linker is attached at position 2. In some embodiments, a linker is attached at position 1 and an antisense oligonucleotide of the invention is attached at position 2.

In some embodiments, the physiologically labile linker comprises or consists of a DNA dinucleotide with a sequence selected from the group consisting of AA, AT, AC, AG, TA, TT, TC, TG, CA, CT, CC, CG, GA, GT, GC, or GG, where there is a phosphodiester linkage between the two DNA nucleosides and at least one further phosphodiester at the 5′ or 3′ end of the dinucleotide linking either the oligonucleotide of the nucleic acid molecule to the dinucleotide or the conjugate moiety to the dinucleotide. For example, the linker may by a CA dinucleotide. In some embodiments, the physiologically labile linker comprises or consists of a DNA trinucleotide of sequence AAA, AAT, AAC, AAG, ATA, ATT, ATC, ATG, ACA, ACT, ACC, ACG, AGA, AGT, AGC, AGG, TAA, TAT, TAC, TAG, TTA, TTT, TTC, TAG, TCA, TCT, TCC, TCG, TGA, TGT, TGC, TGG, CAA, CAT, CAC, CAG, CTA, CTG, CTC, CTT, CCA, CCT, CCC, CCG, CGA, CGT, CGC, CGG, GAA, GAT, GAC, CAG, GTA, GTT, GTC, GTG, GCA, GCT, GCC, GCG, GGA, GGT, GGC, or GGG, where there are phosphodiester linkages between the DNA nucleosides and potentially a further phosphodiester at the 5′ or 3′ end of the trinucleotide. Phosphodiester containing biocleavable linkers are described in more detail in WO 2014/076195 (hereby incorporated by reference). In a conjugate compound with a biocleavable linker at least about 50% of the conjugate moiety is cleaved from the oligonucleotide, such as at least about 60% cleaved, such as at least about 70% cleaved, such as at least about 80% cleaved, such as at least about 85% cleaved, such as at least about 90% cleaved, such as at least about 95% of the conjugate moiety is cleaved from the oligonucleotide cleaved when compared against a standard.

In some embodiments, the linker nucleoside sequence is CA. In other words, in some embodiments, the sequence of the linker nucleoside sequence is CA. In some embodiments, the linker nucleotide sequence is 5′-CA-3′. In some embodiments, the linker nucleoside sequence is the dinucleotide CA, wherein the C nucleoside is linked to the conjugate moiety by a phosphodiester bond, the C nucleoside is linked to the A nucleoside by a phosphodiester internucleoside linkage, and the A nucleoside is linked to the 5′ nucleoside of the antisense oligonucleotide of the invention by a phosphodiester internucleoside linkage.

In some embodiments, the linker is a biocleavable linker. Biocleavable linkers comprises or consist of a physiologically labile bond that is cleavable under conditions normally encountered or analogous to those encountered within a mammalian body. Conditions under which physiologically labile linkers undergo chemical transformation (e.g., cleavage) include chemical conditions such as pH, temperature, oxidative or reductive conditions or agents, and salt concentration found in or analogous to those encountered in mammalian cells. Mammalian intracellular conditions also include the presence of enzymatic activity normally present in a mammalian cell such as from proteolytic enzymes or hydrolytic enzymes or nucleases. In one embodiment the biocleavable linker is susceptible to S1 nuclease cleavage. In some embodiments the nuclease susceptible linker comprises between 1 and 5 nucleosides, such as DNA nucleoside(s) comprising at least two consecutive phosphodiester linkages. Phosphodiester containing biocleavable linkers are described in more detail in WO 2014/076195.

Exemplary Conjugates of the Invention

The term “conjugate” may be used herein to refer to the combination of sequence (i.e. the order of nucleobases), sugar moieties, internucleoside linkages and conjugate moiety in a given antisense oligonucleotide conjugate of the invention. For a given conjugate, the nucleobase, the type of sugar moiety and the internucleoside linkages of each nucleotide in the antisense oligonucleotide of the conjugate are specified. Thus, the order of sugar moieties and internucleoside linkages is also specified in a given conjugate. However, unless otherwise indicated, a conjugate may comprise other elements in addition to the specified sequence, sugar moieties, internucleoside linkages and conjugate moiety.

Specific conjugates are referred to herein using a conjugate identifier number (CNJ ID NO) of the form X_Y, wherein X and Y are each a number. For each CNJ ID NO, X is the number of the SEQ ID NO that corresponds to the sequence of the antisense oligonucleotide component of the conjugate. For example, the conjugate designated CNJ ID NO 2_1 has the same nucleobase sequence as SEQ ID NO 2 (and the same nucleobase sequence as CMP ID NO 2_1).

In some embodiments, the antisense oligonucleotide conjugate of the invention comprises any one of CNJ ID NOs 1_1, 2_1, 3_1, 4_1, 4_2, 5_1, 5_2, 6_1, 7_1, 7_2, 8_1, 2_2, 9_1, 10_1, 11_1, 4_3, 12_1, 5_3, 13_1, 14_1, 15_1, 16_1, 16_2, 16_3, 2_3, 2_4, 2_5, 2_6, 2_7, 2_8, 2_9, 2_10, 2_11, 2_12, 17_1, 18_1, 19_1, 20_1, 20_2, 203, 20_4, 20_5, 20_6, 6_2, 6_3, 6_4, 6_5, 6_6, 6_7, 6_8, 6_9, 6_10, 6_11, 6_12, 21_1, 22_1, 23_1, 24_1, 25_1, 25_2, 26_1, 27_1, 27_2, 28_1, 29_1, 30_1, 11_2 and 11_3. In some embodiments, the antisense oligonucleotide conjugate of the invention consists of any one of CNJ ID NOs 1_1, 2_1, 3_1, 4_1, 4_2, 5_1, 5_2, 6_1, 7_1, 7_2, 8_1, 2_2, 9_1, 10_1, 11_1, 4_3, 12_1, 5_3, 13_1, 14_1, 151, 16_1, 16_2, 16_3, 2_3, 2_4, 2_5, 2_6, 2_7, 2_8, 2_9, 2_10, 2_11, 2_12, 17_1, 18_1, 19_1, 20_1, 20_2, 20_3, 20_4, 20_5, 20_6, 6_2, 6_3, 6_4, 6_5, 6_6, 6_7, 6_8, 6_9, 6_10, 6_11, 6_12, 21_1, 22_1, 23_1, 24_1, 25_1, 25_2, 26_1, 27_1, 27_2, 28_1, 29_1, 30_1, 11_2 and 11_3.

In some embodiments, the antisense oligonucleotide conjugate of the invention comprises any one of CNJ ID NOs 1_1, 2_1, 3_1, 4_1, 4_2, 5_1, 5_2, 6_1, 7_1, 7_2, 8_1, 2_2, 9_1, 10_1, 11_1, 4_3, 12_1, 5_3, 13_1 and 14_1. In some embodiments, the antisense oligonucleotide conjugate of the invention consists of any one of CNJ ID NOs 1_1, 2_1, 3_1, 4_1, 4_2, 5_1, 5_2, 6_1, 7_1, 7_2, 8_1, 2_2, 9_1, 10_1, 11_1, 4_3, 12_1, 5_3, 13_1 and 14_1.

In some embodiments, the antisense oligonucleotide conjugate of the invention comprises or consists of any one of CNJ ID NOs 1_1, 2_1, 3_1, 4_1, 4_2, 5_1 and 5_2. In some embodiments, the antisense oligonucleotide conjugate of the invention comprises any one of CNJ ID NOs 1_1, 2_1, 3_1, 4_1, 4_2, 5_1 and 5_2. In some embodiments, the antisense oligonucleotide conjugate of the invention consists of any one of CNJ ID NOs 1_1, 2_1, 3_1, 4_1, 4_2, 5_1 and 5_2.

The invention provides an antisense oligonucleotide conjugate selected from the following:

(a)
[5gn2c6]P.[dR](C)P.[dR](A)P.[LR](A)[sP].[LR](G)[sP].[dR](G)[sP].[dR](C)[sP].[LR]
(A)[sP].[dR](T)[sP].[dR](T)[sP].[dR](A)[sP].[dR](T)[sP].[dR](A)[sP].[dR](G)[sP].[dR](G)
[sP].[dR](T)[sP].[dR](T)[sP].[dR](T)[sP].[LR](T)[sP].[LR](T)[sP].[LR]([5meC])
(SEQ ID NO 1, CMP ID NO 1_1, CNJ ID NO 1_1),
(b)
[5gn2c6]P.[dR](C)P.[dR](A)P.[LR](A)[PS2].[LR](A)[sP].[dR](A)[sP].[mR](A)[sP].[LR]
(G)[sP].[dR](T)[sP].[dR](T)[sP].[dR](T)[sP].[dR](C)[sP].[dR](T)[sP].[dR](C)[sP].[dR](A)
[sP].[dR](G)[sP].[dR](A)[sP].[dR](T)[sP].[dR](A)[sP].[mR](G)[sP].[LR](G)[PS2].[LR]
([5meC]) (SEQ ID NO 2, CMP ID NO 2_1, CNJ ID NO 2_1),
(c)
[5gn2c6]P.[dR](C)P.[dR](A)P.[LR](G)[sP].[LR](G)[sP].[mR](A)[sP].[dR](C)[sP].[dR]
(A)[sP].[dR](T)[sP].[LR](G)[sP].[dR](T)[sP].[dR](T)[sP].[dR](A)[sP].[dR](A)[sP].[dR](T)
[sP].[dR](T)[sP].[dR](T)[sP].[dR](T)[P].[dR](A)[sP].[dR](C)[sP].[LR](T)[PS2].[LR](T)
(SEQ ID NO 3, CMP ID NO 3_1, CNJ ID NO 3_1),
(d)
[5gn2c6]P.[dR](C)P.[dR](A)P.[LR](T)[PS2].[LR](G)[sP].[LR](A)[sP].[dR](A)[sP].[dR]
(C)[sP].[dR](T)[sP].[dR](T)[sP].[dR](G)[sP].[dR](T)[sP].[dR](T)[sP].[dR](A)[sP].[dR](T)
[sP].[dR](A)[sP].[dR](T)[sP].[dR](A)[sP].[dR](C)[sP].[LR]([5meC])[PS2].[LR](T)[PS2].
[LR](G) (SEQ ID NO 4, CMP ID NO 4_1, CNJ ID NO 4_1),
(e)
[5gn2c6]P.[dR](C)P.[dR](A)P.[LR](T)[PS2].[LR](G)[sP].[LR](A)[sP].[mR](A)[sP].[dR]
(C)[sP].[dR](T)[sP].[dR](T)[sP].[dR](G)[sP].[dR](T)[sP].[dR](T)[sP].[dR](A)[sP].[dR](T)
[sP].[dR](A)[sP].[dR](T)[sP].[dR](A)[sP].[dR](C)[sP].[LR]([5meC])[sP].[LR](T)[PS2].
[LR](G) (SEQ ID NO 4, CMP ID NO 4_2, CNJ ID NO 4_2),
(f)
[5gn2c6]P.[dR](C)P.[R](A)P.[LR](T)[PS2].[LR](A)[PS2].[dR](G)[sP].[dR](A)[sP].
[LR](G)[sP].[dR](T)[sP].[dR](T)[sP].[dR](T)[sP].[dR](T)[sP].[dR](G)[sP].[dR](T)[sP].
[dR](G)[sP].[dR](T)[sP].[dR](G)[sP].[dR](T)[sP].[MOE]([5meC])[sP].[MOE](T)
(SEQ ID NO 5, CMP ID NO 5_1, CNJ ID NO 5_1),
(g)
[5gn2c6]P.[dR](C)P.[dR](A)P.[LR](T)[PS2].[LR](A)[sP].[dR](G)[sP].[dR](A)[sP].[LR]
(G)[sP].[dR](T)[sP].[dR](T)[sP].[dR](T)[sP].[dR](T)[sP].[dR](G)[sP].[dR](T)[sP].[dR](G)
[sP].[dR](T)[sP].[dR](G)[sP].[dR](T)[sP].[MOE]([5meC])[sP].[MOE](T)
(SEQ ID NO 5, CMP ID NO 5_2, CNJ ID NO 5_2),
(h)
[5gn2c6]P.[dR](C)P.[dR](A)P.[LR](G)[sP].[LR]([5meC])[sP].[dR](A)[sP].[dR](T)[sP].
[dR](T)[sP].[dR](A)[sP].[dR](T)[sP].[dR](A)[sP].[dR](G)[sP].[dR](G)[sP].[dR](T)[sP].
[dR](T)[sP].[dR](T)[sP].[LR](T)[sP].[LR](T)[sP].[LR]([5meC])[sP].[LR](T)
(SEQ ID NO 6, CMP ID NO 6_1, CNJ ID NO 6_1),
(i)
[5gn2c6]P.[dR](C)P.[dR](A)P.[MOE](G)[sP].[LR](G)[sP].[dR](C)[sP].[LR](A)[sP].[dR]
(T)[sP].[dR](T)[sP].[dR](A)[sP].[dR](T)[sP].[dR](A)[sP].[dR](G)[sP].[dR](G)[sP].[dR](T)
[sP].[dR](T)[sP].[LR](T)[sP].[MOE](T)[sP].[MOE](T)[sP].[MOE]([5meC])
(SEQ ID NO 7, CMP ID NO 7_1, CNJ ID NO 7_1),
(j)
[5gn2c6]P.[dR](C)P.[dR](A)P.[LR](G)[sP].[LR](G)[sP].[dR](C)[sP].[LR](A)[sP].[dR](T)
[sP].[dR](T)[sP].[dR](A)[sP].[dR](T)[sP].[mR](A)[sP].[dR](G)[sP].[dR](G)[sP].[dR](T)
[sP].[dR](T)[sP].[dR](T)[sP].[LR](T)[sP].[LR](T)[sP].[LR]([5meC])
(SEQ ID NO 7, CMP ID NO 7_2, CNJ ID NO 7_2),
(k)
[5gn2c6]P.[dR](C)P.[dR](A)P.[MOE](G)[sP].[MOE](G)[sP].[MOE] ([5meC])[sP].[LR](A)
[PS2].[dR](T)[sP].[dR](T)[sP].[dR](A)[sP].[dR](T)[sP].[mR](A)[sP].[dR](G)[sP].[dR](G)
[sP].[dR](T)[sP].[dR](T)[sP].[LR](T)[PS2].[LR](T)[PS2].[LR](T)[PS2].[MOE]([5meC])
(SEQ ID NO 8, CMP ID NO 8_1, CNJ ID NO 8_1),
(l)
[5gn2c6]P.[dR](C)P.[dR](A)P.[LR](A)[PS2].[LR](A)[PS2].[dR](A)[sP].[mR](A)[sP].[LR]
(G)[sP].[dR](T)[sP].[dR](T)[sP].[dR](T)[sP].[dR](C)[sP].[dR](T)[sP].[dR](C)[sP].[dR](A)
[sP].[dR](G)[sP].[dR](A)[sP].[dR](T)[sP].[dR](A)[P].[dR](G)[sP].[LR](G)[PS2].[LR]
([5meC]) (SEQ ID NO 2, CMP ID NO 2_2, CNJ ID NO 2_2),
(m)
[5gn2c6]P.[dR](C)P.[dR](A)P.[LR](T)[PS2].[LR](G)[sP].[dR](G)[sP].[LR](A)[sP].[dR]
(C)[sP].[dR](A)[sP].[mR](U)[sP].[dR](G)[sP].[dR](T)[sP].[LR](T)[sP].[dR](A)[sP].[dR]
(A)[sP].[dR](T)[sP].[dR](T)[sP].[dR](T)[sP].[dR](T)[sP].[LR](A)[PS2].[LR]([5meC])
(SEQ ID NO 9, CMP ID NO 9_1, CNJ ID NO 9_1),
(n)
[5gn2c6]P.[dR](C)P.[dR](A)P.[LR](G)[PS2].[LR](G)[PS2].[dR](A)[sP].[dR](C)[sP].[dR]
(A)[sP].[dR](T)[sP].[LR](G)[sP].[mR](U)[sP].[dR](T)[sP].[mR](A)[sP].[dR](A)[sP].[dR]
(T)[P].[dR](T)[sP].[dR](T)[sP].[dR](T)[sP].[dR](A)[sP].[dR](C)[sP].[LR](T)[PS2].[LR]
(T) (SEQ ID NO 10, CMP ID NO 10_1, CNJ ID NO 10_1),
(o)
[5gn2c6]P.[dR](C)P.[dR](A)P.[LR](G)[sP].[LR](T)[sP].[LR](T)[sP].[dR](T)[sP].[dR](T)
[sP].[mR](A)[sP].[dR](A)[sP].[dR](G)[sP].[dR](A)[sP].[dR](T)[sP].[dR](T)[sP].[dR](G)
[sP].[dR](T)[sP].[dR](G)[sP].[dR](G)[sP].[dR](T)[sP].[dR](G)[sP].[LR](T)[sP].[LR](T)
[PS2].[LR](T) (SEQ ID NO 11, CMP ID NO 11_1, CNJ ID NO 11_1),
(p)
[5gn2c6]P.[dR](C)P.[dR](A)P.[LR](T)[PS2].[LR](G)[P].[LR](A)[sP].[dR](A)[sP].[dR](C)
[sP].[dR](T)[sP].[dR](T)[sP].[dR](G)[sP].[dR](T)[sP].[dR](T)[sP].[dR](A)[sP].[dR](T)
[sP].[dR](A)[sP].[dR](T)[sP].[dR](A)[sP].[dR](C)[sP].[LR]([5meC])[sP].[LR](T)[PS2].[LR]
(G) (SEQ ID NO 4, CMP ID NO 4_3, CNJ ID NO 4_3),
(q)
[5gn2c6]P.[dR](C)P.[dR](A)P.[LR](T)[PS2].[LR](A)[PS2].[dR](G)[sP].[dR](A)[sP].[LR]
(G)[sP].[dR](T)[sP].[dR](T)[sP].[dR](T)[sP].[mR](U)[sP].[dR](G)[sP].[dR](T)[sP].[dR]
(G)[sP].[dR](T)[sP].[dR](G)[sP].[dR](T)[sP].[MOE]([5meC])[sP].[MOE](T)
(SEQ ID NO 12, CMP ID NO 12_1, CNJ ID NO 12_1),
(r)
[5gn2c6]P.[dR](C)P.[dR](A)P.[MOE](T)[sP].[MOE](A)[sP].[MOE](G)[sP].[MOE](A)[sP].
[LR](G)[PS2].[dR](T)[sP].[dR](T)[sP].[dR](T)[sP].[dR](T)[sP].[dR](G)[sP].[dR](T)[sP].
[dR](G)[sP].[dR](T)[sP].[dR](G)[sP].[dR](T)[sP].[LR]([5meC])[PS2].[MOE](T)
(SEQ ID NO 5, CMP ID NO 5_3, CNJ ID NO 5_3),
(s)
[5gn2c6]P.[dR](C)P.[dR](A)P.[MOE](T)[sP].[MOE](A)[sP].[MOE](G)[sP].[MOE](A)[sP].
[LR](G)[PS2].[dR](T)[sP].[dR](T)[sP].[dR](T)[sP].[dR](T)[sP].[dR](G)[sP].[dR](T)[sP].
[dR](G)[sP].[mR](U)[sP].[dR](G)[sP].[dR](T)[sP].[LR]([5meC])[PS2].[MOE](T)
(SEQ ID NO 13, CMP ID NO 13_1, CNJ ID NO 13_1),
(t)
[5gn2c6]P.[dR](C)P.[dR](A)P.[LR](A)[PS2].[LR](T)[sP].[LR](A)[sP].[LR](G)[sP].[dR](A)
[sP].[dR]([PPG])[sP].[dR](T)[sP].[dR](T)[sP].[dR](T)[sP].[dR](T)[sP].[dR](G)[sP].[dR]
(T)[sP].[dR](G)[sP].[dR](T)[sP].[dR](G)[P].[LR](T)[PS2].[LR]([5meC])
(SEQ ID NO 14, CMP ID NO 14_1, CNJ ID NO 14_1),
(u)
[5gn2c6]P.[dR](C)P.[dR](A)P.[LR](A)[PS2].[LR](A)[sP].[LR](A)[sP].[dR](A)[sP].[dR]
([PPG])[sP].[dR](T)[sP].[dR](T)[sP].[dR](T)[sP].[dR](C)[sP].[dR](T)[sP].[dR](C)[sP].
[dR](A)[sP].[dR](G)[P].[dR](A)[sP].[dR](T)[sP].[dR](A)[sP].[LR](G)[sP].[LR](G)[PS2].
[LR]([5meC]) (SEQ ID NO 15, CMP ID NO 15_1, CNJ ID NO 15_1),
(v)
[5gn2c6]P.[dR](C)P.[dR](A)P.[LR](A)[PS2].[LR](A)[sP].[LR](A)[sP].[dR](A)[sP].[dR](G)
[sP].[dR](T)[sP].[dR](T)[sP].[dR](T)[sP].[dR](C)[sP].[dR](T)[sP].[dR](C)[sP].[dR](A)
[sP].[dR]([PPG])[sP].[dR](A)[sP].[dR](T)[sP].[dR](A)[P].[LR](G)[sP].[LR](G)[PS2].[LR]
([5meC]) (SEQ ID NO 16, CMP ID NO 16_1, CNJ ID NO 16_1),
(w)
[5gn2c6]P.[dR](C)P.[dR](A)P.[MOE](A)[sP].[LR](A)[PS2].[LR](A)[sP].[dR](A)[sP].[dR]
(G)[sP].[dR](T)[sP].[dR](T)[sP].[dR](T)[sP].[dR](C)[sP].[dR](T)[sP].[dR](C)[sP].[dR](A)
[sP].[dR]([PPG])[sP].[dR](A)[P].[dR](T)[sP].[dR](A)[sP].[LR](G)[sP].[LR](G)[PS2].
[MOE]([5meC]) (SEQ ID NO 16, CMP ID NO 16_2, CNJ ID NO 16_2),
(x)
[5gn2c6]P.[dR](C)P.[dR](A)P.[MOE](A)[sP].[LR](A)[PS2].[LR](A)[sP].[dR](A)[sP].[dR]
(G)[sP].[dR](T)[sP].[dR](T)[sP].[dR](T)[sP].[dR](C)[sP].[dR](T)[sP].[dR](C)[sP].[dR](A)
[sP].[dR]([PPG])[sP].[dR](A)[sP].[dR](T)[sP].[dR](A)[sP].[LR](G)[PS2].[LR](G)[PS2].
[MOE]([5meC]) (SEQ ID NO 16, CMP ID NO 16_3, CNJ ID NO 16_3),
(y)
[5gn2c6]P.[dR](C)P.[dR](A)P.[LR](A)[PS2].[LR](A)[sP].[dR](A)[sP].[mR](A)[sP].[LR]
(G)[sP].[dR](T)[sP].[dR](T)[sP].[dR](T)[sP].[dR](C)[sP].[dR](T)[sP].[dR](C)[P].[dR](A)
[sP].[dR](G)[sP].[dR](A)[P].[dR](T)[sP].[dR](A)[sP].[dR](G)[sP].[LR](G)[PS2].[LR]
([5meC]) (SEQ ID NO 2, CMP ID NO 2_3, CNJ ID NO 2_3),
(z)
[5gn2c6]P.[dR](C)P.[dR](A)P.[LR](A)[PS2].[LR](A)[PS2].[dR](A)[sP].[mR](A)[sP].[LR]
(G)[sP].[dR](T)[sP].[dR](T)[sP].[dR](T)[sP].[dR](C)[sP].[dR](T)[sP].[dR](C)[sP].[dR](A)
[sP].[dR](G)[sP].[dR](A)[sP].[dR](T)[sP].[dR](A)[sP].[mR](G)[sP].[LR](G)[PS2].[LR]
([5meC]) (SEQ ID NO 2, CMP ID NO 2_4, CNJ ID NO 2_4),
(aa)
[5gn2c6]P.[dR](C)P.[dR](A)P.[LR](A)[PS2].[LR](A)[sP].[LR](A)[sP].[dR](A)[sP].[dR](G)
[sP].[dR](T)[sP].[dR](T)[sP].[dR](T)[sP].[dR](C)[sP].[dR](T)[sP].[dR](C)[sP].[mR](A)
[sP].[dR](G)[sP].[dR](A)[P].[dR](T)[sP].[dR](A)[sP].[LR](G)[sP].[LR](G)[PS2].[LR]
([5meC]) (SEQ ID NO 2, CMP ID NO 2_5, CNJ ID NO 2_5),
(ab)
[5gn2c6]P.[dR](C)P.[dR](A)P.[LR](A)[PS2].[MOE](A)[sP].[MOE](A)[sP].[dR](A)[sP].
[dR](G)[sP].[dR](T)[sP].[dR](T)[sP].[dR](T)[sP].[dR](C)[sP].[dR](T)[sP].[dR](C)[sP].
[mR](A)[sP].[dR](G)[sP].[dR](A)[sP].[dR](T)[sP].[dR](A)[sP].[LR](G)[PS2].[LR](G)
[PS2].[MOE]([5meC]) (SEQ ID NO 2, CMP ID NO 2_6, CNJ ID NO 2_6),
(ac)
[5gn2c6]P.[dR](C)P.[dR](A)P.[LR](A)[PS2].[LR](A)[P].[LR](A)[sP].[dR](A)[sP].[dR](G)
[sP].[dR](T)[sP].[dR](T)[sP].[dR](T)[sP].[dR](C)[sP].[dR](T)[sP].[mR](C)[sP].[dR](A)
[sP].[dR](G)[sP].[dR](A)[sP].[dR](T)[sP].[dR](A)[sP].[LR](G)[sP].[LR](G)[PS2].[LR]
([5meC]) (SEQ ID NO 2, CMP ID NO 2_7, CNJ ID NO 2_7),
(ad)
[5gn2c6]P.[dR](C)P.[dR](A)P.[MOE](A)[sP].[MOE](A)[sP].[LR](A)[PS2].[dR](A)[sP].
[dR](G)[sP].[dR](T)[sP].[dR](T)[sP].[dR](T)[sP].[dR](C)[P].[dR](T)[sP].[mR](C)[sP].
[dR](A)[sP].[dR](G)[sP].[dR](A)[sP].[dR](T)[sP].[dR](A)[sP].[LR](G)[sP].[LR](G)[PS2].
[MOE]([5meC]) (SEQ ID NO 2, CMP ID NO 2_8, CNJ ID NO 2_8),
(ae)
[5gn2c6]P.[dR](C)P.[dR](A)P.[MOE](A)[sP].[LR](A)[P].[LR](A)[sP].[dR](A)[sP].[dR]
(G)[sP].[dR](T)[sP].[dR](T)[sP].[dR](T)[sP].[dR](C)[sP].[dR](T)[sP].[dR](C)[sP].[mR]
(A)[sP].[dR](G)[sP].[dR](A)[P].[dR](T)[sP].[dR](A)[sP].[LR](G)[sP].[LR](G)[sP].[MOE]
([5meC]) (SEQ ID NO 2, CMP ID NO 2_9, CNJ ID NO 2_9),
(af)
[5gn2c6]P.[dR](C)P.[dR](A)P.[LR](A)[PS2].[LR](A)[sP].[LR](A)[sP].[dR](A)[sP].[dR](G)
[sP].[dR](T)[sP].[dR](T)[sP].[dR](T)[sP].[mR](C)[sP].[dR](T)[sP].[dR](C)[sP].[dR](A)
[sP].[dR](G)[sP].[dR](A)[sP].[dR](T)[sP].[dR](A)[sP].[LR](G)[sP].[LR](G)[PS2].[LR]
([5meC]) (SEQ ID NO 2, CMP ID NO 2_10, CNJ ID NO 2_10),
(ag)
[5gn2c6]P.[dR](C)P.[dR](A)P.[MOE](A)[sP].[LR](A)[sP].[LR](A)[sP].[dR](A)[sP].[dR]
(G)[sP].[dR](T)[sP].[dR](T)[sP].[dR](T)[sP].[mR](C)[sP].[dR](T)[sP].[dR](C)[sP].[dR]
(A)[sP].[dR](G)[sP].[dR](A)[sP].[dR](T)[sP].[dR](A)[sP].[LR](G)[sP].[LR](G)[PS2].
[MOE]([5meC]) (SEQ ID NO 2, CMP ID NO 2_11, CNJ ID NO 2_11),
(ah)
[5gn2c6]P.[dR](C)P.[dR](A)P.[MOE](A)[sP].[LR](A)[PS2].[LR](A)[sP].[dR](A)[sP].[dR]
(G)[sP].[dR](T)[sP].[dR](T)[sP].[dR](T)[sP].[dR](C)[sP].[dR](T)[sP].[dR](C)[sP].[dR](A)
[sP].[dR](G)[sP].[dR](A)[sP].[dR](T)[sP].[mR](A)[sP].[LR](G)[sP].[LR](G)[PS2].[MOE]
([5meC]) (SEQ ID NO 2, CMP ID NO 2_12, CNJ ID NO 2_12),
(ai)
[5gn2c6]P.[dR](C)P.[dR](A)P.[MOE](A)[sP].[MOE](A)[sP].[LR](A)[sP].[dR](A)[sP].[dR]
(G)[sP].[dR](T)[sP].[dR](T)[sP].[mR](U)[sP].[dR](C)[sP].[dR](T)[sP].[dR](C)[sP].[dR]
(A)[sP].[dR](G)[sP].[dR](A)[sP].[dR](T)[sP].[dR](A)[P].[LR](G)[sP].[LR](G)[PS2].
[MOE]([5meC]) (SEQ ID NO 17, CMP ID NO 17_1, CNJ ID NO 17_1),
(aj)
[5gn2c6]P.[dR](C)P.[dR](A)P.[LR](A)[PS2].[LR](G)[sP].[dR](G)[sP].[dR](C)[sP].[LR]
(A)[sP].[dR](T)[sP].[dR](T)[sP].[dR](A)[sP].[dR](T)[sP].[dR](A)[sP].[dR](G)[sP].[dR](G)
[sP].[mR](U)[sP].[dR](T)[sP].[dR](T)[sP].[LR](T)[sP].[LR](T)[PS2].[LR]([5meC])
(SEQ ID NO 18, CMP ID NO 18_1, CNJ ID NO 18_1),
(ak)
[5gn2c6]P.[dR](C)P.[dR](A)P.[LR](A)[PS2].[LR](G)[sP].[dR](G)[sP].[dR](C)[P].[LR]
(A)[sP].[mR](U)[sP].[dR](T)[sP].[dR](A)[sP].[dR](T)[sP].[dR](A)[sP].[dR](G)[sP].[dR]
(G)[sP].[dR](T)[sP].[dR](T)[sP].[dR](T)[sP].[LR](T)[sP].[LR](T)[PS2].[LR]([5meC])
(SEQ ID NO 19, CMP ID NO 19_1, CNJ ID NO 19_1),
(al)
[5gn2c6]P.[dR](C)P.[dR](A)P.[LR](G)[PS2].[LR]([5meC])[sP].[dR](A)[sP].[dR](T)[sP].
[dR](T)[sP].[dR](A)[sP].[dR](T)[sP].[dR](A)[sP].[dR](G)[sP].[dR](G)[sP].[dR](T)[sP].
[LR](T)[sP].[dR](T)[sP].[dR](T)[sP].[dR](T)[sP].[dR](C)[sP].[LR](T)[PS2].[LR]([5meC])
(SEQ ID NO 20, CMP ID NO 20_1, CNJ ID NO 20_1),
(am)
[5gn2c6]P.[dR](C)P.[dR](A)P.[LR](G)[PS2].[LR]([5meC])[sP].[mR](A)[sP].[dR](T)[sP].
[dR](T)[sP].[dR](A)[sP].[dR](T)[sP].[dR](A)[sP].[dR](G)[sP].[dR](G)[sP].[dR](T)[sP].
[LR](T)[sP].[dR](T)[sP].[dR](T)[sP].[dR](T)[sP].[dR](C)[sP].[LR](T)[PS2].[LR]([5meC])
(SEQ ID NO 20, CMP ID NO 20_2, CNJ ID NO 20_2),
(an)
[5gn2c6]P.[dR](C)P.[dR](A)P.[LR](G)[PS2].[LR]([5meC])[sP].[dR](A)[sP].[dR](T)[sP].
[dR](T)[sP].[dR](A)[sP].[dR](T)[sP].[mR](A)[sP].[dR](G)[sP].[dR](G)[sP].[dR](T)[sP].
[LR](T)[sP].[dR](T)[sP].[dR](T)[sP].[dR](T)[sP].[dR](C)[sP].[LR](T)[PS2].[LR]([5meC])
(SEQ ID NO 20, CMP ID NO 20_3, CNJ ID NO 20_3),
(ao)
[5gn2c6]P.[dR](C)P.[dR](A)P.[LR](G)[PS2].[LR]([5meC])[sP].[dR](A)[sP].[dR](T)[sP].
[dR](T)[sP].[dR](A)[sP].[dR](T)[sP].[dR](A)[sP].[mR](G)[sP].[dR](G)[sP].[dR](T)[sP].
[LR](T)[sP].[dR](T)[sP].[dR](T)[sP].[dR](T)[sP].[dR](C)[sP].[LR](T)[PS2].[LR]([5meC])
(SEQ ID NO 20, CMP ID NO 20_4, CNJ ID NO 20_4),
(ap)
[5gn2c6]P.[dR](C)P.[dR](A)P.[LR](G)[PS2].[LR]([5meC])[sP].[dR](A)[P].[dR](T)[sP].
[dR](T)[sP].[dR](A)[sP].[dR](T)[sP].[dR](A)[sP].[dR](G)[sP].[dR](G)[sP].[dR](T)[sP].
[LR](T)[sP].[dR](T)[sP].[dR](T)[P].[dR](T)[sP].[dR](C)[sP].[LR](T)[PS2].[MOE]
([5meC]) (SEQ ID NO 20, CMP ID NO 20_5, CNJ ID NO 20_5),
(aq)
[5gn2c6]P.[dR](C)P.[dR](A)P.[LR](G)[PS2].[LR]([5meC])[sP].[dR](A)[sP].[dR](T)[sP].
[dR](T)[P].[dR](A)[sP].[dR](T)[sP].[dR](A)[sP].[dR](G)[sP].[mR](G)[sP].[dR](T)[sP].
[LR](T)[sP].[dR](T)[sP].[dR](T)[sP].[dR](T)[sP].[dR](C)[sP].[LR](T)[PS2].[LR]([5meC])
(SEQ ID NO 20, CMP ID NO 20_6, CNJ ID NO 20_6),
(ar)
[5gn2c6]P.[dR](C)P.[dR](A)P.[LR](G)[PS2].[LR]([5meC])[sP].[LR](A)[sP].[dR](T)[sP].
[dR](T)[sP].[dR](A)[sP].[dR](T)[sP].[dR](A)[sP].[dR](G)[sP].[dR](G)[sP].[dR](T)[sP].
[dR](T)[sP].[dR](T)[sP].[LR](T)[sP].[dR](T)[sP].[LR]([5meC])[PS2].[LR](T)
(SEQ ID NO 6, CMP ID NO 6_2, CNJ ID NO 6_2),
(as)
[5gn2c6]P.[dR](C)P.[dR](A)P.[LR](G)[PS2].[LR]([5meC])[sP].[LR](A)[sP].[dR](T)[sP].
[dR](T)[sP].[dR](A)[sP].[dR](T)[sP].[mR](A)[sP].[dR](G)[sP].[dR](G)[sP].[dR](T)[sP].
[dR](T)[sP].[dR](T)[sP].[LR](T)[P].[dR](T)[sP].[LR]([5meC])[PS2].[LR](T)
(SEQ ID NO 6, CMP ID NO 6_3, CNJ ID NO 6_3),
(at)
[5gn2c6]P.[dR](C)P.[dR](A)P.[LR](G)[PS2].[LR]([5meC])[sP].[LR](A)[sP].[dR](T)[sP].
[dR](T)[sP].[dR](A)[sP].[dR](T)[sP].[dR](A)[sP].[mR](G)[sP].[dR](G)[sP].[dR](T)[sP].
[dR](T)[sP].[dR](T)[sP].[LR](T)[sP].[dR](T)[sP].[LR]([5meC])[PS2].[LR](T)
(SEQ ID NO 6, CMP ID NO 6_4, CNJ ID NO 6_4),
(au)
[5gn2c6]P.[dR](C)P.[dR](A)P.[LR](G)[PS2].[LR]([5meC])[sP].[mR](A)[sP].[dR](T)[sP].
[dR](T)[sP].[dR](A)[sP].[dR](T)[sP].[dR](A)[sP].[dR](G)[sP].[dR](G)[sP].[dR](T)[sP].
[dR](T)[sP].[dR](T)[sP].[LR](T)[sP].[LR](T)[sP].[LR]([5meC])[PS2].[LR](T)
(SEQ ID NO 6, CMP ID NO 6_5, CNJ ID NO 6_5),
(av)
[5gn2c6]P.[dR](C)P.[dR](A)P.[LR](G)[PS2].[LR]([5meC])[sP].[dR](A)[sP].[dR](T)[sP].
[dR](T)[sP].[dR](A)[sP].[dR](T)[sP].[mR](A)[sP].[dR](G)[P].[dR](G)[sP].[dR](T)[sP].
[dR](T)[P].[dR](T)[sP].[LR](T)[sP].[LR](T)[sP].[LR]([5meC])[PS2].[LR](T)
(SEQ ID NO 6, CMP ID NO 6_6, CNJ ID NO 6_6),
(aw)
[5gn2c6]P.[dR](C)P.[dR](A)P.[LR](G)[PS2].[LR]([5meC])[sP].[dR](A)[sP].[dR](T)[sP].
[dR](T)[sP].[dR](A)[sP].[dR](T)[sP].[dR](A)[sP].[mR](G)[sP].[dR](G)[sP].[dR](T)[sP].
[dR](T)[sP].[dR](T)[sP].[LR](T)[sP].[LR](T)[sP].[LR]([5meC])[PS2].[LR](T)
(SEQ ID NO 6, CMP ID NO 6_7, CNJ ID NO 6_7),
(ax)
[5gn2c6]P.[dR](C)P.[dR](A)P.[LR](G)[PS2].[LR]([5meC])[sP].[dR](A)[sP].[dR](T)[sP].
[dR](T)[sP].[dR](A)[sP].[dR](T)[sP].[dR](A)[sP].[dR](G)[sP].[mR](G)[sP].[dR](T)[sP].
[dR](T)[sP].[dR](T)[sP].[LR](T)[sP].[LR](T)[sP].[LR]([5meC])[PS2].[LR](T)
(SEQ ID NO 6, CMP ID NO 6_8, CNJ ID NO 6_8),
(ay)
[5gn2c6]P.[dR](C)P.[dR](A)P.[MOE](G)[sP].[LR]([5meC])[PS2].[dR](A)[sP].[dR](T)
[sP].[dR](T)[sP].[dR](A)[sP].[dR](T)[sP].[dR](A)[sP].[dR](G)[sP].[dR](G)[sP].[dR](T)
[sP].[dR](T)[sP].[dR](T)[sP].[LR](T)[sP].[LR](T)[sP].[LR]([5meC])[PS2].[MOE](T)
(SEQ ID NO 6, CMP ID NO 6_9, CNJ ID NO 6_9),
(az)
[5gn2c6]P.[dR](C)P.[dR](A)P.[MOE](G)[sP].[LR]([5meC])[PS2].[dR](A)[sP].[dR](T)
[sP].[dR](T)[sP].[dR](A)[sP].[dR](T)[sP].[dR](A)[sP].[dR](G)[sP].[dR](G)[sP].[dR](T)
[sP].[dR](T)[sP].[dR](T)[sP].[LR](T)[sP].[LR](T)[PS2].[MOE]([5meC])[sP].[MOE](T)
(SEQ ID NO 6, CMP ID NO 6_10, CNJ ID NO 6_10),
(ba)
[5gn2c6]P.[dR](C)P.[dR](A)P.[MOE](G)[sP].[LR]([5meC])[PS2].[dR](A)[sP].[dR](T)
[sP].[dR](T)[sP].[dR](A)[sP].[dR](T)[sP].[dR](A)[sP].[dR](G)[sP].[dR](G)[sP].[dR](T)
[sP].[dR](T)[sP].[dR](T)[sP].[LR](T)[sP].[LR](T)[PS2].[LR]([5meC])[PS2].[MOE](T)
(SEQ ID NO 6, CMP ID NO 6_11, CNJ ID NO 6_11),
(bb)
[5gn2c6]P.[dR](C)P.[dR](A)P.[MOE](G)[sP].[LR]([5meC])[PS2].[dR](A)[sP].[dR](T)
[sP].[dR](T)[sP].[dR](A)[sP].[dR](T)[sP].[dR](A)[sP].[dR](G)[sP].[dR](G)[sP].[dR](T)
[sP].[dR](T)[sP].[dR](T)[sP].[LR](T)[PS2].[LR](T)[PS2].[MOE]([5meC])[sP].[MOE](T)
(SEQ ID NO 6, CMP ID NO 6_12, CNJ ID NO 6_12),
(bc)
[5gn2c6]P.[dR](C)P.[dR](A)P.[LR](G)[PS2].[LR]([5meC])[sP].[dR](A)[sP].[dR](T)[sP].
[dR](T)[sP].[dR](A)[sP].[dR](T)[sP].[dR](A)[sP].[dR](G)[sP].[dR](G)[sP].[dR](T)[sP].
[LR](T)[sP].[dR](T)[sP].[dR](T)[P].[mR](U)[sP].[dR](C)[sP].[LR](T)[PS2].[LR]([5meC])
(SEQ ID NO 21, CMP ID NO 21_1, CNJ ID NO 21_1),
(bd)
[5gn2c6]P.[dR](C)P.[dR](A)P.[LR](G)[PS2].[LR]([5meC])[sP].[dR](A)[sP].[dR](T)[sP].
[dR](T)[sP].[dR](A)[sP].[dR](T)[sP].[dR](A)[sP].[dR](G)[sP].[dR](G)[sP].[dR](T)[sP].
[LR](T)[P].[dR](T)[sP].[mR](U)[sP].[dR](T)[sP].[dR](C)[sP].[LR](T)[PS2].[LR]([5meC])
(SEQ ID NO 22, CMP ID NO 22_1, CNJ ID NO 22_1),
(be)
[5gn2c6]P.[dR](C)P.[dR](A)P.[LR](G)[PS2].[LR]([5meC])[P].[dR](A)[sP].[dR](T)[sP].
[dR](T)[sP].[dR](A)[sP].[dR](T)[sP].[dR](A)[sP].[dR](G)[sP].[dR](G)[sP].[dR](T)[sP].
[LR](T)[sP].[mR](U)[sP].[dR](T)[sP].[dR](T)[sP].[dR](C)[sP].[LR](T)[PS2].[LR]([5meC])
(SEQ ID NO 23, CMP ID NO 23_1, CNJ ID NO 23_1),
(bf)
[5gn2c6]P.[dR](C)P.[dR](A)P.[LR](G)[PS2].[LR]([5meC])[sP].[dR](A)[sP].[dR](T)[sP].
[dR](T)[sP].[dR](A)[P].[dR](T)[sP].[dR](A)[sP].[dR](G)[sP].[dR](G)[sP].[dR](T)[sP].
[dR](T)[sP].[mR](U)[sP].[LR](T)[sP].[LR](T)[sP].[LR]([5meC])[PS2].[LR](T)
(SEQ ID NO 24, CMP ID NO 24_1, CNJ ID NO 24_1),
(bg)
[5gn2c6]P.[dR](C)P.[dR](A)P.[LR](G)[PS2].[LR]([5meC])[sP].[LR](A)[sP].[dR](T)[sP].
[dR](T)[sP].[dR](A)[sP].[dR](T)[sP].[dR](A)[sP].[dR](G)[sP].[dR](G)[sP].[dR](T)[sP].
[mR](U)[sP].[dR](T)[sP].[LR](T)[sP].[dR](T)[sP].[LR]([5meC])[PS2].[LR](T)
(SEQ ID NO 25, CMP ID NO 25_1, CNJ ID NO 25_1),
(bh)
[5gn2c6]P.[dR](C)P.[dR](A)P.[LR](G)[PS2].[LR]([5meC])[sP].[dR](A)[sP].[dR](T)[sP].
[dR](T)[sP].[dR](A)[sP].[dR](T)[sP].[dR](A)[sP].[dR](G)[sP].[dR](G)[sP].[dR](T)[sP].
[mR](U)[sP].[dR](T)[P].[LR](T)[sP].[LR](T)[P].[LR]([5meC])[PS2].[LR](T)
(SEQ ID NO 25, CMP ID NO 25_2, CNJ ID NO 25_2),
(bi)
[5gn2c6]P.[dR](C)P.[dR](A)P.[LR](G)[PS2].[LR]([5meC])[sP].[dR](A)[sP].[dR](T)[sP].
[dR](T)[sP].[dR](A)[sP].[dR](T)[sP].[dR](A)[sP].[dR](G)[sP].[dR](G)[sP].[mR](U)[sP].
[LR](T)[sP].[dR](T)[sP].[dR](T)[sP].[dR](T)[sP].[dR](C)[sP].[LR](T)[PS2].[LR]([5meC])
(SEQ ID NO 26, CMP ID NO 26_1, CNJ ID NO 26_1),
(bj)
[5gn2c6]P.[dR](C)P.[dR](A)P.[LR](G)[PS2].[LR]([5meC])[sP].[LR](A)[sP].[dR](T)[sP].
[dR](T)[sP].[dR](A)[sP].[dR](T)[sP].[dR](A)[sP].[dR](G)[sP].[dR](G)[sP].[mR](U)[sP].
[dR](T)[sP].[dR](T)[sP].[LR](T)[sP].[dR](T)[sP].[LR]([5meC])[PS2].[LR](T)
(SEQ ID NO 27, CMP ID NO 27_1, CNJ ID NO 27_1),
(bk)
[5gn2c6]P.[dR](C)P.[dR](A)P.[LR](G)[PS2].[LR]([5meC])[sP].[dR](A)[sP].[dR](T)[sP].
[dR](T)[sP].[dR](A)[P].[dR](T)[sP].[dR](A)[sP].[dR](G)[P].[dR](G)[sP].[mR](U)[sP].
[dR](T)[sP].[dR](T)[sP].[LR](T)[sP].[LR](T)[P].[LR]([5meC])[PS2].[LR](T)
(SEQ ID NO 27, CMP ID NO 27_2, CNJ ID NO 27_2),
(bl)
[5gn2c6]P.[dR](C)P.[dR](A)P.[LR](G)[PS2].[LR]([5meC])[sP].[dR](A)[sP].[dR](T)[sP].
[dR](T)[sP].[dR](A)[sP].[mR](U)[sP].[dR](A)[sP].[dR](G)[sP].[dR](G)[sP].[dR](T)[sP].
[dR](T)[sP].[dR](T)[sP].[LR](T)[sP].[LR](T)[sP].[LR]([5meC])[PS2].[LR](T)
(SEQ ID NO 28, CMP ID NO 28_1, CNJ ID NO 28_1),
(bm)
[5gn2c6]P.[dR](C)P.[dR](A)P.[LR](G)[PS2].[LR]([5meC])[sP].[dR](A)[sP].[dR](T)[sP].
[mR](U)[sP].[dR](A)[sP].[dR](T)[sP].[dR](A)[sP].[dR](G)[sP].[dR](G)[sP].[dR](T)[sP].
[dR](T)[sP].[dR](T)[sP].[LR](T)[sP].[LR](T)[sP].[LR]([5meC])[PS2].[LR](T)
(SEQ ID NO 29, CMP ID NO 29_1, CNJ ID NO 29_1),
(bn)
[5gn2c6]P.[dR](C)P.[dR](A)P.[LR](G)[PS2].[LR]([5meC])[sP].[dR](A)[sP].[mR](U)[sP].
[dR](T)[sP].[dR](A)[sP].[dR](T)[sP].[dR](A)[sP].[dR](G)[sP].[dR](G)[sP].[dR](T)[sP].
[dR](T)[sP].[dR](T)[sP].[LR](T)[sP].[LR](T)[sP].[LR]([5meC])[PS2].[LR](T)
(SEQ ID NO 30, CMP ID NO 30_1, CNJ ID NO 30_1),
(bo)
[5gn2c6]P.[dR](C)P.[dR](A)P.[LR](G)[sP].[LR](T)[sP].[LR](T)[sP].[dR](T)[sP].[dR](T)
[sP].[dR](A)[sP].[dR](A)[sP].[mR](G)[sP].[dR](A)[sP].[dR](T)[sP].[dR](T)[sP].[dR](G)
[sP].[dR](T)[sP].[dR](G)[sP].[dR](G)[sP].[dR](T)[sP].[dR](G)[sP].[LR](T)[sP].[LR](T)
[sP].[LR](T) (SEQ ID NO 11, CMP ID NO 11_2, CNJ ID NO 11_2), and
(bp)
[5gn2c6]P.[dR](C)P.[dR](A)P.[LR](G)[sP].[LR](T)[sP].[LR](T)[sP].[dR](T)[sP].[dR](T)
[sP].[dR](A)[sP].[dR](A)[sP].[mR](G)[sP].[dR](A)[sP].[dR](T)[sP].[dR](T)[sP].[dR](G)
[sP].[dR](T)[sP].[dR](G)[sP].[dR](G)[sP].[dR](T)[sP].[dR](G)[sP].[LR](T)[sP].[LR](T)
[PS2].[LR](T) (SEQ ID NO 11, CMP ID NO 11_3, CNJ ID NO 11_3),

wherein
(A) is a nucleoside comprising an adenine nucleobase,
(C) is a nucleoside comprising a cytosine nucleobase,
([5meC]) is a nucleoside comprising a 5-methyl cytosine nucleobase,
([PPG]) is a nucleoside comprising a 7-deaza-8-aza guanine nucleobase,
(G) is a nucleoside comprising a guanine nucleobase,
(U) is a nucleoside comprising a uracil nucleobase,
(T) is a nucleoside comprising a thymine nucleobase,
[LR] indicates that the following nucleoside is a beta-D-oxy-LNA nucleoside,
[dR] indicates that the following nucleoside is a DNA nucleoside,
[mR] indicates that the following nucleoside is a 2′-O-methyl RNA nucleoside,
[MOE] indicates that the following nucleoside is a 2′-O-methoxyethyl RNA (MOE RNA) nucleoside,
[sP] indicates a phosphorothioate internucleoside linkage between the preceding and following nucleoside,
[PS2] indicates a phosphorodithioate internucleoside linkage between the preceding and following nucleoside,
P indicates a phosphodiester internucleoside linkage between the preceding nucleoside or moiety and the following nucleoside, and
[5gn2c6] is an aminohexyl conjugated tri(N-acetyl-galactosamine) moiety as shown in FIG. 8.

The invention also provides an antisense oligonucleotide conjugate selected from the following:

(a)
[5gn2c6]P.[dR](C)P.[dR](A)P.[LR](A)[sP].[LR](G)[sP].[dR](G)[sP].[dR](C)[sP].[LR]
(A)[sP].[dR](T)[sP].[dR](T)[sP].[dR](A)[sP].[dR](T)[sP].[dR](A)[sP].[dR](G)[sP].[dR](G)
[sP].[dR](T)[sP].[dR](T)[sP].[dR](T)[sP].[LR](T)[sP].[LR](T)[sP].[LR]([5meC])
(SEQ ID NO 1, CMP ID NO 1_1, CNJ ID NO 1_1),
(b)
[5gn2c6]P.[dR](C)P.[dR](A)P.[LR](A)[PS2].[LR](A)[sP].[dR](A)[sP].[mR](A)[sP].[LR]
(G)[sP].[dR](T)[P].[dR](T)[sP].[dR](T)[sP].[dR](C)[sP].[dR](T)[sP].[dR](C)[sP].[dR](A)
[sP].[dR](G)[sP].[dR](A)[sP].[dR](T)[sP].[dR](A)[sP].[mR](G)[sP].[LR](G)[PS2].[LR]
([5meC]) (SEQ ID NO 2, CMP ID NO 2_1, CNJ ID NO 2_1),
(c)
[5gn2c6]P.[dR](C)P.[dR](A)P.[LR](G)[P].[LR](G)[sP].[mR](A)[sP].[dR](C)[sP].[dR]
(A)[sP].[dR](T)[sP].[LR](G)[sP].[dR](T)[sP].[dR](T)[sP].[dR](A)[sP].[dR](A)[sP].[dR](T)
[sP].[dR](T)[sP].[dR](T)[sP].[dR](T)[sP].[dR](A)[sP].[dR](C)[sP].[LR](T)[PS2].[LR](T)
(SEQ ID NO 3, CMP ID NO 3_1, CNJ ID NO 3_1),
(d)
[5gn2c6]P.[dR](C)P.[dR](A)P.[LR](T)[PS2].[LR](G)[sP].[LR](A)[sP].[dR](A)[sP].[dR]
(C)[sP].[dR](T)[sP].[dR](T)[P].[dR](G)[sP].[dR](T)[sP].[dR](T)[sP].[dR](A)[sP].[dR](T)
[sP].[dR](A)[sP].[dR](T)[sP].[dR](A)[sP].[dR](C)[sP].[LR]([5meC])[PS2].[LR](T)[PS2].
[LR](G) (SEQ ID NO 4, CMP ID NO 4_1, CNJ ID NO 4_1),
(e)
[5gn2c6]P.[dR](C)P.[dR](A)P.[LR](T)[PS2].[LR](G)[sP].[LR](A)[sP].[mR](A)[sP].[dR]
(C)[sP].[dR](T)[sP].[dR](T)[sP].[dR](G)[sP].[dR](T)[sP].[dR](T)[sP].[dR](A)[sP].[dR](T)
[sP].[dR](A)[sP].[dR](T)[sP].[dR](A)[sP].[dR](C)[sP].[LR]([5meC])[sP].[LR](T)[PS2].
[LR](G) (SEQ ID NO 4, CMP ID NO 4_2, CNJ ID NO 4_2),
(f)
[5gn2c6]P.[dR](C)P.[dR](A)P.[LR](T)[PS2].[LR](A)[PS2].[dR](G)[sP].[dR](A)[sP].
[LR](G)[sP].[dR](T)[sP].[dR](T)[sP].[dR](T)[sP].[dR](T)[P].[dR](G)[sP].[dR](T)[sP].
[dR](G)[sP].[dR](T)[sP].[dR](G)[sP].[dR](T)[sP].[MOE]([5meC])[sP].[MOE](T)
(SEQ ID NO 5, CMP ID NO 5_1, CNJ ID NO 5_1),
(g)
[5gn2c6]P.[dR](C)P.[dR](A)P.[LR](T)[PS2].[LR](A)[sP].[dR](G)[sP].[dR](A)[sP].[LR]
(G)[sP].[dR](T)[sP].[dR](T)[sP].[dR](T)[sP].[dR](T)[sP].[dR](G)[P].[dR](T)[sP].[dR](G)
[sP].[dR](T)[sP].[dR](G)[sP].[dR](T)[sP].[MOE]([5meC])[P].[MOE](T)
(SEQ ID NO 5, CMP ID NO 5_2, CNJ ID NO 5_2),
(h)
[5gn2c6]P.[dR](C)P.[dR](A)P.[LR](G)[sP].[LR]([5meC])[sP].[dR](A)[sP].[dR](T)[sP].
[dR](T)[sP].[dR](A)[sP].[dR](T)[sP].[dR](A)[sP].[dR](G)[sP].[dR](G)[sP].[dR](T)[sP].
[dR](T)[sP].[dR](T)[sP].[LR](T)[sP].[LR](T)[sP].[LR]([5meC])[sP].[LR](T)
(SEQ ID NO 6, CMP ID NO 6_1, CNJ ID NO 6_1),
(i)
[5gn2c6]P.[dR](C)P.[dR](A)P.[MOE](G)[sP].[LR](G)[sP].[dR](C)[sP].[LR](A)[sP].[dR]
(T)[sP].[dR](T)[sP].[dR](A)[sP].[dR](T)[sP].[dR](A)[sP].[dR](G)[sP].[dR](G)[sP].[dR](T)
[sP].[dR](T)[sP].[LR](T)[sP].[MOE](T)[sP].[MOE](T)[sP].[MOE] ([5meC])
(SEQ ID NO 7, CMP ID NO 7_1, CNJ ID NO 7_1),
(j)
[5gn2c6]P.[dR](C)P.[dR](A)P.[LR](G)[sP].[LR](G)[sP].[dR](C)[sP].[LR](A)[P].[dR](T)
[sP].[dR](T)[sP].[dR](A)[sP].[dR](T)[sP].[mR](A)[sP].[dR](G)[sP].[dR](G)[sP].[dR](T)
[sP].[dR](T)[sP].[dR](T)[sP].[LR](T)[sP].[LR](T)[sP].[LR]([5meC])
(SEQ ID NO 7, CMP ID NO 7_2, CNJ ID NO 7_2),
(k)
[5gn2c6]P.[dR](C)P.[dR](A)P.[MOE](G)[sP].[MOE](G)[sP].[MOE]([5meC])[sP].[LR](A)
[PS2].[dR](T)[sP].[dR](T)[sP].[dR](A)[sP].[dR](T)[sP].[mR](A)[sP].[dR](G)[sP].[dR](G)
[sP].[dR](T)[sP].[dR](T)[sP].[LR](T)[PS2].[LR](T)[PS2].[LR](T)[PS2].[MOE]([5meC])
(SEQ ID NO 8, CMP ID NO 8_1, CNJ ID NO 8_1),
(l)
[5gn2c6]P.[dR](C)P.[dR](A)P.[LR](A)[PS2].[LR](A)[PS2].[dR](A)[sP].[mR](A)[sP].[LR]
(G)[sP].[dR](T)[sP].[dR](T)[sP].[dR](T)[sP].[dR](C)[sP].[dR](T)[sP].[dR](C)[sP].[dR](A)
[sP].[dR](G)[sP].[dR](A)[sP].[dR](T)[sP].[dR](A)[sP].[dR](G)[sP].[LR](G)[PS2].[LR]
([5meC]) (SEQ ID NO 2, CMP ID NO 2_2, CNJ ID NO 2_2),
(m)
[5gn2c6]P.[dR](C)P.[dR](A)P.[LR](T)[PS2].[LR](G)[sP].[dR](G)[sP].[LR](A)[sP].[dR]
(C)[sP].[dR](A)[sP].[mR](U)[sP].[dR](G)[sP].[dR](T)[sP].[LR](T)[sP].[dR](A)[sP].[dR]
(A)[sP].[dR](T)[P].[dR](T)[sP].[dR](T)[sP].[dR](T)[sP].[LR](A)[PS2].[LR] ([5meC])
(SEQ ID NO 9, CMP ID NO 9_1, CNJ ID NO 9_1),
(n)
[5gn2c6]P.[dR](C)P.[dR](A)P.[LR](G)[PS2].[LR](G)[PS2].[dR](A)[sP].[dR](C)[sP].[dR]
(A)[sP].[dR](T)[sP].[LR](G)[sP].[mR](U)[sP].[dR](T)[sP].[mR](A)[sP].[dR](A)[sP].[dR]
(T)[sP].[dR](T)[sP].[dR](T)[sP].[dR](T)[sP].[dR](A)[sP].[dR](C)[sP].[LR](T)[PS2].[LR]
(T) (SEQ ID NO 10, CMP ID NO 10_1, CNJ ID NO 10_1),
(o)
[5gn2c6]P.[dR](C)P.[dR](A)P.[LR](G)[sP].[LR](T)[sP].[LR](T)[sP].[dR](T)[sP].[dR](T)
[sP].[mR](A)[sP].[dR](A)[sP].[dR](G)[sP].[dR](A)[sP].[dR](T)[sP].[dR](T)[sP].[dR](G)
[sP].[dR](T)[sP].[dR](G)[sP].[dR](G)[sP].[dR](T)[sP].[dR](G)[sP].[LR](T)[sP].[LR](T)
[PS2].[LR](T) (SEQ ID NO 11, CMP ID NO 11_1, CNJ ID NO 11_1),
(p)
[5gn2c6]P.[dR](C)P.[dR](A)P.[LR](T)[PS2].[LR](G)[sP].[LR](A)[sP].[dR](A)[sP].[dR](C)
[sP].[dR](T)[sP].[dR](T)[sP].[dR](G)[sP].[dR](T)[sP].[dR](T)[sP].[dR](A)[sP].[dR](T)
[sP].[dR](A)[sP].[dR](T)[sP].[dR](A)[sP].[dR](C)[P].[LR]([5meC])[sP].[LR](T)[PS2].[LR]
(G) (SEQ ID NO 4, CMP ID NO 4_3, CNJ ID NO 4_3),
(q)
[5gn2c6]P.[dR](C)P.[dR](A)P.[LR](T)[PS2].[LR](A)[PS2].[dR](G)[sP].[dR](A)[sP].[LR]
(G)[sP].[dR](T)[sP].[dR](T)[sP].[dR](T)[sP].[mR](U)[sP].[dR](G)[sP].[dR](T)[sP].[dR]
(G)[sP].[dR](T)[sP].[dR](G)[sP].[dR](T)[sP].[MOE]([5meC])[sP].[MOE](T)
(SEQ ID NO 12, CMP ID NO 12_1, CNJ ID NO 12_1),
(r)
[5gn2c6]P.[dR](C)P.[dR](A)P.[MOE](T)[sP].[MOE](A)[sP].[MOE](G)[sP].[MOE](A)[sP].
[LR](G)[PS2].[dR](T)[sP].[dR](T)[sP].[dR](T)[sP].[dR](T)[sP].[dR](G)[P].[dR](T)[sP].
[dR](G)[sP].[dR](T)[sP].[dR](G)[sP].[dR](T)[sP].[LR]([5meC])[PS2].[MOE](T)
(SEQ ID NO 5, CMP ID NO 5_3, CNJ ID NO 5_3),
(s)
[5gn2c6]P.[dR](C)P.[dR](A)P.[MOE](T)[P].[MOE](A)[sP].[MOE](G)[sP].[MOE](A)[sP].
[LR](G)[PS2].[dR](T)[sP].[dR](T)[sP].[dR](T)[sP].[dR](T)[sP].[dR](G)[sP].[dR](T)[sP].
[dR](G)[sP].[mR](U)[sP].[dR](G)[sP].[dR](T)[sP].[LR]([5meC])[PS2].[MOE](T)
(SEQ ID NO 13, CMP ID NO 13_1, CNJ ID NO 13_1),
and
(t)
[5gn2c6]P.[dR](C)P.[dR](A)P.[LR](A)[PS2].[LR](T)[sP].[LR](A)[sP].[LR](G)[sP].[dR](A)
[sP].[dR]([PPG])[sP].[dR](T)[sP].[dR](T)[sP].[dR](T)[sP].[dR](T)[sP].[dR](G)[sP].[dR]
(T)[sP].[dR](G)[sP].[dR](T)[sP].[dR](G)[sP].[LR](T)[PS2].[LR]([5meC])
(SEQ ID NO 14, CMP ID NO 14_1, CNJ ID NO 14_1),

wherein
(A) is a nucleoside comprising an adenine nucleobase,
(C) is a nucleoside comprising a cytosine nucleobase,
([5meC]) is a nucleoside comprising a 5-methyl cytosine nucleobase,
([PPG]) is a nucleoside comprising a 7-deaza-8-aza guanine nucleobase,
(G) is a nucleoside comprising a guanine nucleobase,
(U) is a nucleoside comprising a uracil nucleobase,
(T) is a nucleoside comprising a thymine nucleobase,
[LR] indicates that the following nucleoside is a beta-D-oxy-LNA nucleoside,
[dR] indicates that the following nucleoside is a DNA nucleoside,
[mR] indicates that the following nucleoside is a 2′-O-methyl RNA nucleoside,
[MOE] indicates that the following nucleoside is a 2′-O-methoxyethyl RNA (MOE RNA) nucleoside,
[sP] indicates a phosphorothioate internucleoside linkage between the preceding and following nucleoside,
[PS2] indicates a phosphorodithioate internucleoside linkage between the preceding and following nucleoside,
P indicates a phosphodiester internucleoside linkage between the preceding nucleoside or moiety and the following nucleoside, and
[5gn2c6] is an aminohexyl conjugated tri(N-acetyl-galactosamine) moiety as shown in FIG. 8.

The invention also provides an antisense oligonucleotide conjugate selected from the following:

(a)
[5gn2c6]P.[dR](C)P.[dR](A)P.[LR](A)[sP].[LR](G)[sP].[dR](G)[sP].[dR](C)[sP].[LR]
(A)[sP].[dR](T)[sP].[dR](T)[sP].[dR](A)[sP].[dR](T)[sP].[dR](A)[sP].[dR](G)[sP].[dR](G)
[sP].[dR](T)[sP].[dR](T)[sP].[dR](T)[sP].[LR](T)[sP].[LR](T)[sP].[LR]([5meC])
(SEQ ID NO 1, CMP ID NO 1_1, CNJ ID NO 1_1),
(b)
[5gn2c6]P.[dR](C)P.[dR](A)P.[LR](A)[PS2].[LR](A)[sP].[dR](A)[sP].[mR](A)[sP].[LR]
(G)[sP].[dR](T)[sP].[dR](T)[sP].[dR](T)[sP].[dR](C)[sP].[dR](T)[sP].[dR](C)[sP].[dR](A)
[sP].[dR](G)[sP].[dR](A)[sP].[dR](T)[sP].[dR](A)[sP].[mR](G)[sP].[LR](G)[PS2].[LR]
([5meC]) (SEQ ID NO 2, CMP ID NO 2_1, CNJ ID NO 2_1),
(c)
[5gn2c6]P.[dR](C)P.[dR](A)P.[LR](G)[sP].[LR](G)[sP].[mR](A)[sP].[dR](C)[sP].[dR]
(A)[sP].[dR](T)[sP].[LR](G)[sP].[dR](T)[sP].[dR](T)[sP].[dR](A)[sP].[dR](A)[sP].[dR](T)
[sP].[dR](T)[sP].[dR](T)[sP].[dR](T)[sP].[dR](A)[sP].[dR](C)[sP].[LR](T)[PS2].[LR](T)
(SEQ ID NO 3, CMP ID NO 3_1, CNJ ID NO 3_1),
(d)
[5gn2c6]P.[dR](C)P.[dR](A)P.[LR](T)[PS2].[LR](G)[sP].[LR](A)[sP].[dR](A)[sP].[dR]
(C)[sP].[dR](T)[sP].[dR](T)[sP].[dR](G)[sP].[dR](T)[sP].[dR](T)[sP].[dR](A)[sP].[dR](T)
[sP].[dR](A)[sP].[dR](T)[sP].[dR](A)[sP].[dR](C)[sP].[LR]([5meC])[PS2].[LR](T)[PS2].
[LR](G) (SEQ ID NO 4, CMP ID NO 4_1, CNJ ID NO 4_1),
(e)
[5gn2c6]P.[dR](C)P.[dR](A)P.[LR](T)[PS2].[LR](G)[sP].[LR](A)[sP].[mR](A)[sP].[dR]
(C)[sP].[dR](T)[sP].[dR](T)[sP].[dR](G)[sP].[dR](T)[sP].[dR](T)[sP].[dR](A)[sP].[dR](T)
[sP].[dR](A)[sP].[dR](T)[sP].[dR](A)[sP].[dR](C)[sP].[LR]([5meC])[sP].[LR](T)[PS2].
[LR](G) (SEQ ID NO 4, CMP ID NO 4_2, CNJ ID NO 4_2),
(f)
[5gn2c6]P.[dR](C)P.[dR](A)P.[LR](T)[PS2].[LR](A)[PS2].[dR](G)[sP].[dR](A)[sP].
[LR](G)[P].[dR](T)[sP].[dR](T)[sP].[dR](T)[sP].[dR](T)[sP].[dR](G)[sP].[dR](T)[sP].
[dR](G)[sP].[dR](T)[sP].[dR](G)[sP].[dR](T)[sP].[MOE]([5meC])[sP].[MOE](T)
(SEQ ID NO 5, CMP ID NO 5_1, CNJ ID NO 5_1), and
(g)
[5gn2c6]P.[dR](C)P.[dR](A)P.[LR](T)[PS2].[LR](A)[sP].[dR](G)[sP].[dR](A)[sP].[LR]
(G)[sP].[dR](T)[sP].[dR](T)[sP].[dR](T)[sP].[dR](T)[sP].[dR](G)[sP].[dR](T)[sP].[dR](G)
[sP].[dR](T)[sP].[dR](G)[sP].[dR](T)[sP].[MOE]([5meC])[sP].[MOE](T)
(SEQ ID NO 5, CMP ID NO 5_2, CNJ ID NO 5_2),

wherein
(A) is a nucleoside comprising an adenine nucleobase,
(C) is a nucleoside comprising a cytosine nucleobase,
([5meC]) is a nucleoside comprising a 5-methyl cytosine nucleobase,
([PPG]) is a nucleoside comprising a 7-deaza-8-aza guanine nucleobase,
(G) is a nucleoside comprising a guanine nucleobase,
(U) is a nucleoside comprising a uracil nucleobase,
(T) is a nucleoside comprising a thymine nucleobase,
[LR] indicates that the following nucleoside is a beta-D-oxy-LNA nucleoside,
[dR] indicates that the following nucleoside is a DNA nucleoside,
[mR] indicates that the following nucleoside is a 2′-O-methyl RNA nucleoside,
[MOE] indicates that the following nucleoside is a 2′-O-methoxyethyl RNA (MOE RNA) nucleoside,
[sP] indicates a phosphorothioate internucleoside linkage between the preceding and following nucleoside,
[PS2] indicates a phosphorodithioate internucleoside linkage between the preceding and following nucleoside,
P indicates a phosphodiester internucleoside linkage between the preceding nucleoside or moiety and the following nucleoside, and
[5gn2c6] is an aminohexyl conjugated tri(N-acetyl-galactosamine) moiety as shown in FIG. 8.

The invention also provides an antisense oligonucleotide conjugate selected from the antisense oligonucleotide conjugates depicted by HELM strings in Table 5 herein.

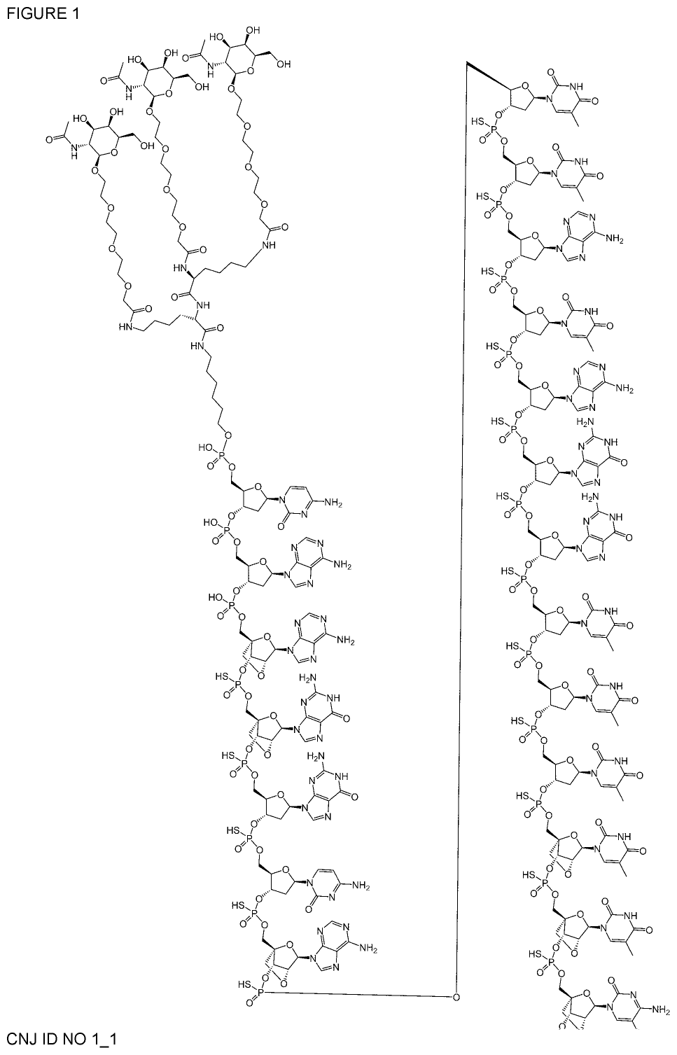

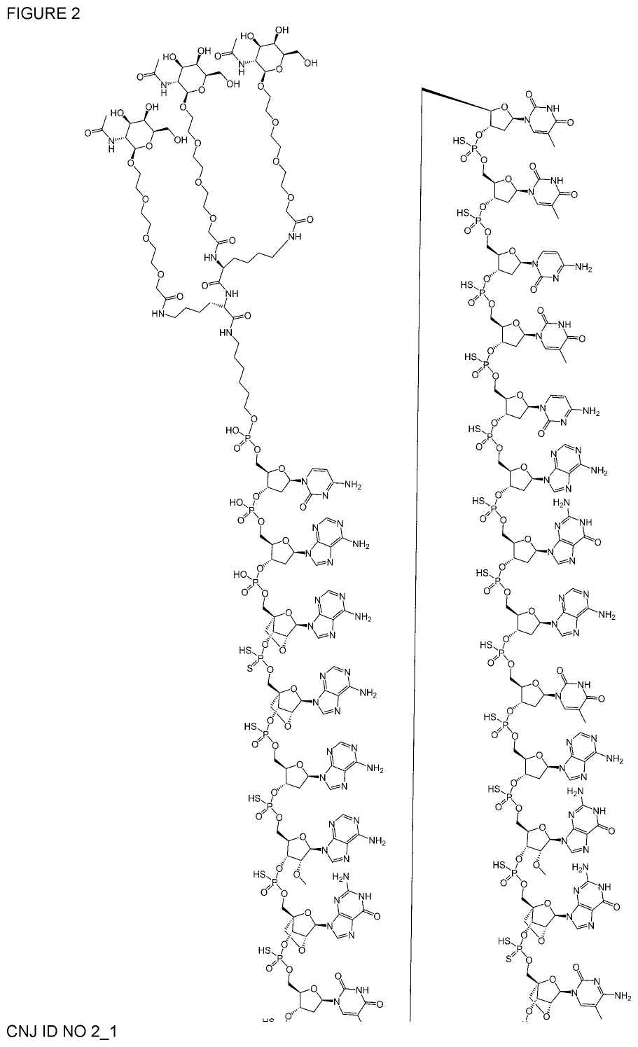

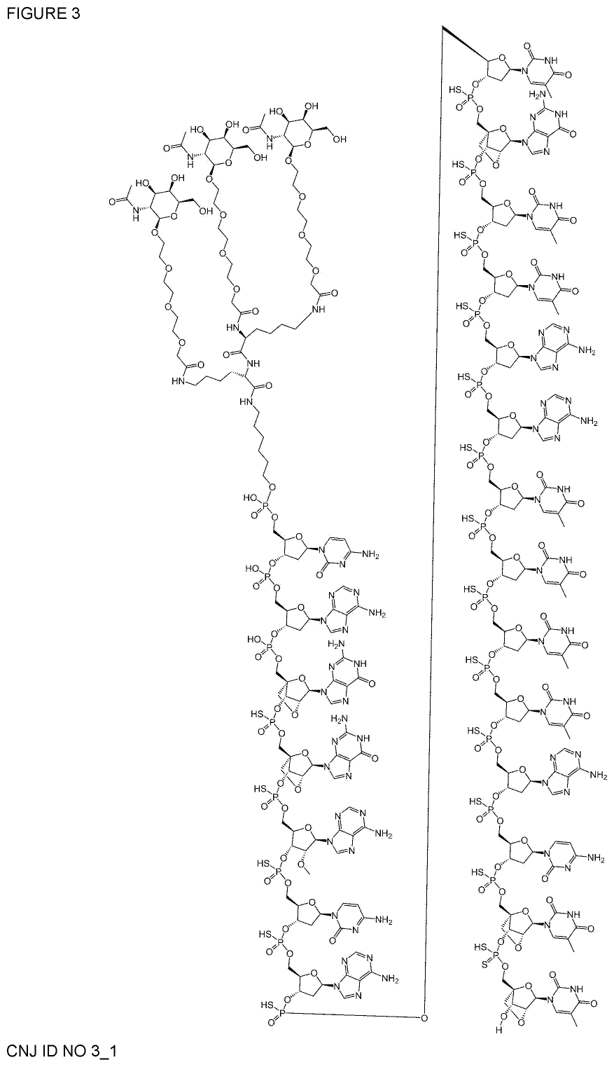

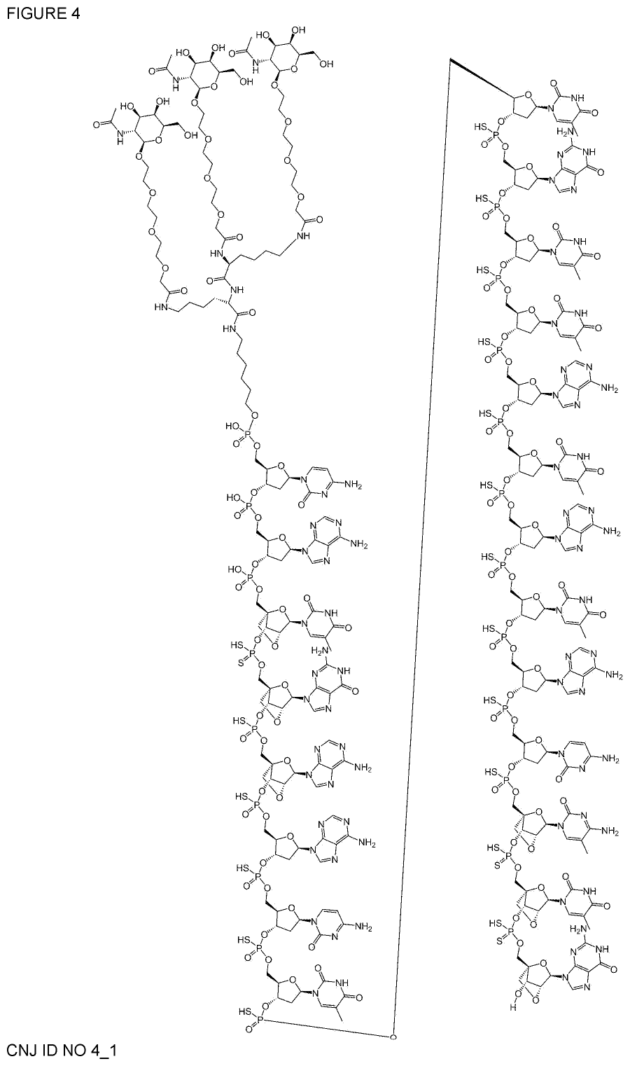

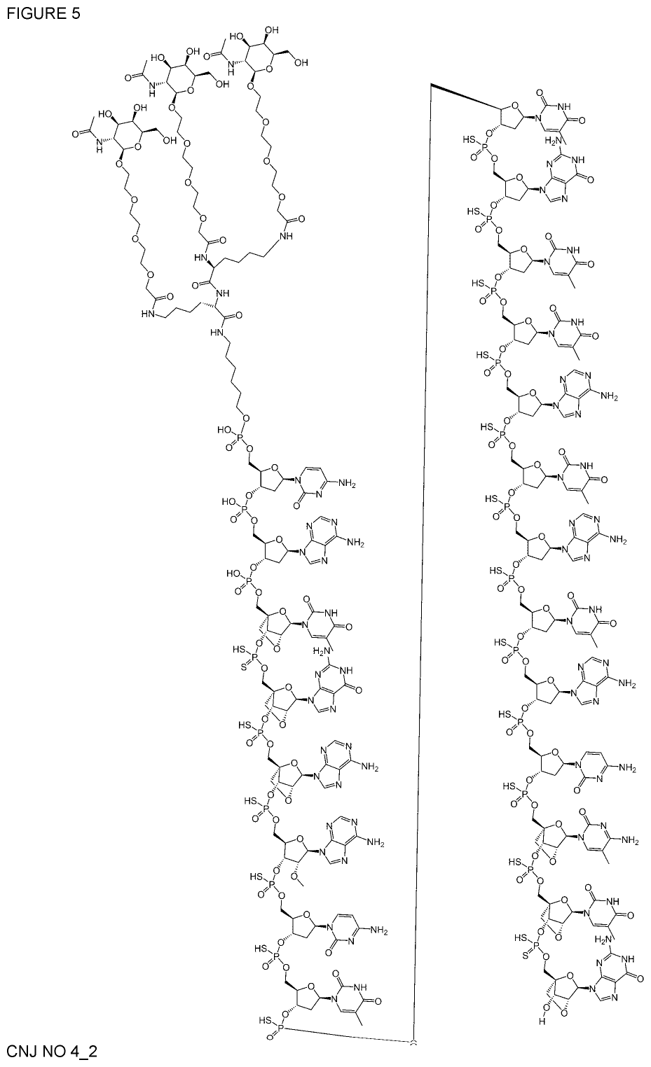

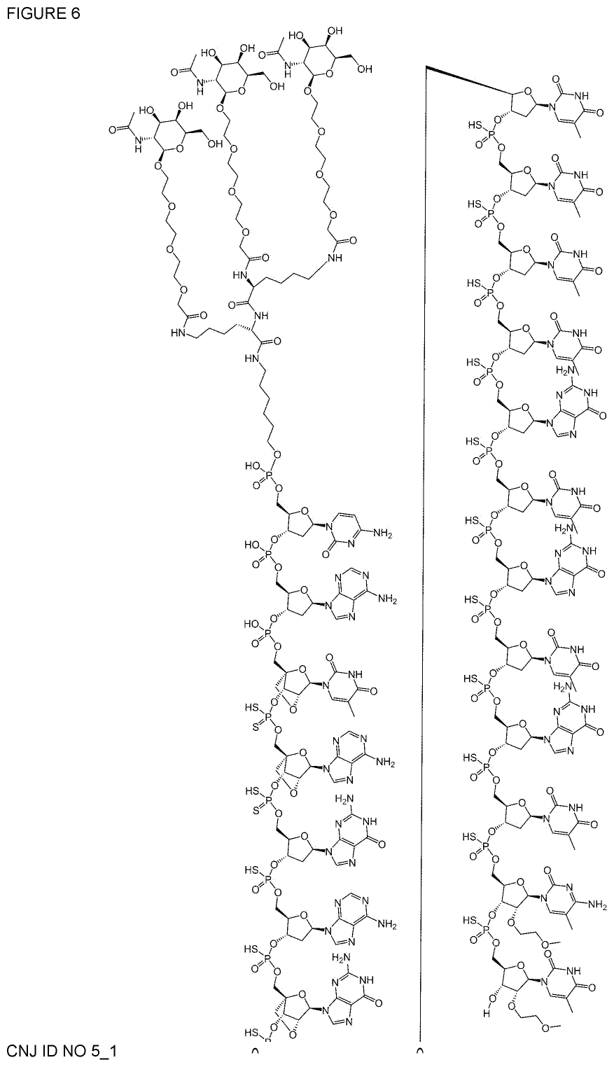

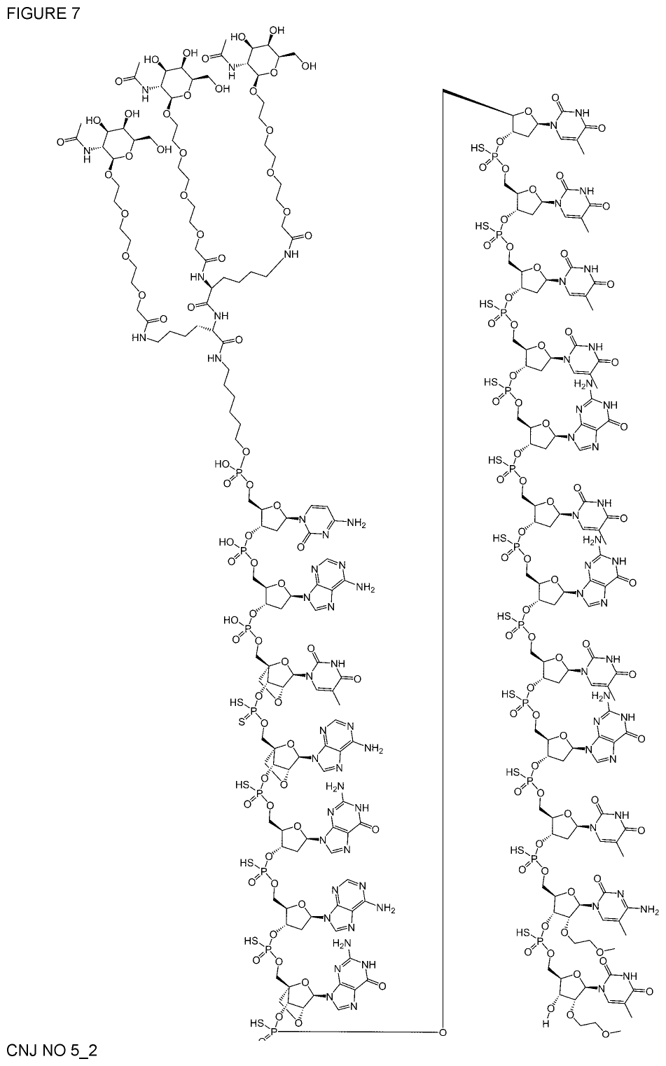

The invention also provides an antisense oligonucleotide conjugate selected from the group consisting of:
(a) CNJ ID NO 1_1 as depicted in FIG. 1;
(b) CNJ ID NO 2_1 as depicted in FIG. 2;
(c) CNJ ID NO 3_1 as depicted in FIG. 3;
(d) CNJ ID NO 4_1 as depicted in FIG. 4;
(e) CNJ ID NO 4_2 as depicted in FIG. 5;
(f) CNJ ID NO 5_1 as depicted in FIG. 6; and
(g) CNJ ID NO 5_2 as depicted in FIG. 7.

Targeting A1CF 2181-2199

The antisense oligonucleotide or antisense oligonucleotide conjugate of the invention may bind a target sequence within the region defined by positions 2181-2199 of the A1CF pre-mRNA (SEQ ID NO 45).

Thus, the invention provides an antisense oligonucleotide of 12 to 30 nucleotides in length, which comprises a contiguous nucleotide sequence of 12 to 30 nucleotides in length, wherein the contiguous nucleotide sequence is capable of binding to a target sequence in an A1CF mRNA, wherein the target sequence is a sequence of at least 17 contiguous nucleotides within GCCUAUCUGAGAAACUUUU (SEQ ID NO 32) (positions 2181-2199 of SEQ ID NO 45), and wherein the antisense oligonucleotide is capable of reducing A1CF expression.

The invention also provides an antisense oligonucleotide conjugate comprising an antisense oligonucleotide covalently attached to at least one conjugate moiety, wherein the antisense oligonucleotide is an antisense oligonucleotide of 12 to 30 nucleotides in length, which comprises a contiguous nucleotide sequence of 12 to 30 nucleotides in length, wherein the contiguous nucleotide sequence is capable of binding to a target sequence in an A1CF mRNA, wherein the target sequence is a sequence of at least 17 contiguous nucleotides within GCCUAUCUGAGAAACUUUU (SEQ ID NO 32) (positions 2181-2199 of SEQ ID NO 45), and wherein the antisense oligonucleotide is capable of reducing A1CF expression.

In some embodiments of the antisense oligonucleotide or the antisense oligonucleotide conjugate of the invention, the target sequence is a sequence of 17, 18, 19 or 20 contiguous nucleotides within SEQ ID NO 32.

In some embodiments, the target sequence is SEQ ID NO 32.

In some embodiments, the sequence of the antisense oligonucleotide and/or the sequence of the contiguous nucleotide sequence comprises or consists of a sequence having at least 80% identity to any one of the following sequences:

| (SEQ ID NO 2) | |
|---|---|
| | AAAAGTTTCTCAGATAGGE, |
| (SEQ ID NO 15) | |
| | AAAAFTTTCTCAGATAGGE, |
| (SEQ ID NO 16) | |
| | AAAAGTTTCTCAFATAGGE |
| | and |
| (SEQ ID NO 17) | |
| | AAAAGTTUCTCAGATAGGE. |

In some embodiments, the sequence of the antisense oligonucleotide and/or the sequence of the contiguous nucleotide sequence comprises or consists of a sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to any one of SEQ ID NOs 2, 15, 16 or 17.

In some embodiments, the sequence of the antisense oligonucleotide and/or the sequence of the contiguous nucleotide sequence comprises or consists of a sequence having at least 80% identity to SEQ ID NO 2.

In some embodiments, the sequence of the antisense oligonucleotide and/or the sequence of the contiguous nucleotide sequence comprises or consists of a sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO 2.

In some embodiments, the sequence of the antisense oligonucleotide and/or the sequence of the contiguous nucleotide sequence consists of SEQ ID NO 2. In some embodiments, the sequence of the sequence of the contiguous nucleotide sequence consists of SEQ ID NO 2. In some embodiments, the sequence of the antisense oligonucleotide consists of SEQ ID NO 2.

In some embodiments, the antisense oligonucleotide comprises or consists of any one of CMP ID NOs 2_1, 2_2, 15_1, 16_1, 162, 163, 2_3, 2_4, 2_5, 2_6, 2_7, 2_8, 2_9, 210, 2_11, 2_12, and 17_1.

In some embodiments, the antisense oligonucleotide comprises or consists of any one of CMP ID NOs 2_1, 2_2, 2_3, 2_4, 2_5, 2_6, 2_7, 2_8, 2_9, 210, 2_11, and 2_12.

In some embodiments, the antisense oligonucleotide comprises or consists of CMP ID NO 2_1 or CMP ID NO 2_2.

In some embodiments, the antisense oligonucleotide comprises or consists of CMP ID NO 2_1.

The invention provides an antisense oligonucleotide comprising or consisting of a compound selected from the following:

| (SEQ ID NO 2, CMP ID NO 2_1) | |
|---|---|
| (a) | [LR](A)[PS2].[LR](A)[sP].[dR](A)[sP].[mR](A)[sP].[LR](G)[sP].[dR](T)[sP].[dR](T)[sP]. |
| | [dR](T)[sP].[dR](C)[sP].[dR](T)[sP].[dR](C)[sP].[dR](A)[sP].[dR](G)[sP].[dR](A)[sP]. |
| | [dR](T)[sP].[dR](A)[sP].[mR](G)[sP].[LR](G)[PS2].[LR]([5meC]), |
| (SEQ ID NO 2, CMP ID NO 2_2) | |
| (b) | [LR](A)[PS2].[LR](A)[PS2].[dR](A)[sP].[mR](A)[sP].[LR](G)[sP].[dR](T)[sP].[dR](T)[sP]. |
| | [dR](T)[sP].[dR](C)[sP].[dR](T)[sP].[dR](C)[sP].[dR](A)[sP].[dR](G)[sP].[dR](A)[sP]. |
| | [dR](T)[sP].[dR](A)[sP].[dR](G)[sP].[LR](G)[PS2].[LR]([5meC]), |
| (SEQ ID NO 16, CMP ID NO 15_1) | |
| (c) | [LR](A)[PS2].[LR](A)[sP].[LR](A)[sP].[dR](A)[sP].[dR]([PPG])[sP].[dR](T)[sP].[dR](T) |
| | [sP].[dR](T)[sP].[dR](C)[sP].[dR](T)[sP].[dR](C)[sP].[dR](A)[sP].[dR](G)[sP].[dR](A) |
| | [sP].[dR](T)[sP].[dR](A)[sP].[LR](G)[sP].[LR](G)[PS2].[LR]([5meC]), |
| (SEQ ID NO 17, CMP ID NO 16_1) | |
| (d) | [LR](A)[PS2].[LR](A)[sP].[LR](A)[sP].[dR](A)[sP].[dR](G)[sP].[dR](T)[sP].[dR](T)[sP]. |
| | [dR](T)[sP].[dR](C)[sP].[dR](T)[sP].[dR](C)[sP].[dR](A)[sP].[dR]([PPG])[sP].[dR](A) |
| | [sP].[dR](T)[sP].[dR](A)[sP].[LR](G)[sP].[LR](G)[PS2].[LR]([5meC]), |
| (SEQ ID NO 17, CMP ID NO 16_2) | |
| (e) | [MOE](A)[P].[LR](A)[PS2].[LR](A)[sP].[dR](A)[sP].[dR](G)[sP].[dR](T)[sP].[dR](T)[sP]. |
| | [dR](T)[sP].[dR](C)[sP].[dR](T)[sP].[dR](C)[sP].[dR](A)[sP].[dR]([PPG])[sP].[dR](A) |
| | [sP].[dR](T)[sP].[dR](A)[sP].[LR](G)[sP].[LR](G)[PS2].[MOE]([5meC]), |
| (SEQ ID NO 17, CMP ID NO 16_3) | |
| (f) | [MOE](A)[sP].[LR](A)[PS2].[LR](A)[sP].[dR](A)[sP].[dR](G)[sP].[dR](T)[sP].[dR](T)[sP]. |
| | [dR](T)[sP].[dR](C)[sP].[dR](T)[sP].[dR](C)[sP].[dR](A)[sP].[dR]([PPG])[sP].[dR](A) |
| | [sP].[dR](T)[sP].[dR](A)[sP].[LR](G)[PS2].[LR](G)[PS2].[MOE]([5meC]), |
| (SEQ ID NO 2, CMP ID NO 2_3) | |
| (g) | [LR](A)[PS2].[LR](A)[sP].[dR](A)[sP].[mR](A)[sP].[LR](G)[sP].[dR](T)[sP].[dR](T)[sP]. |
| | [dR](T)[sP].[dR](C)[sP].[dR](T)[sP].[dR](C)[sP].[dR](A)[sP].[dR](G)[sP].[dR](A)[sP]. |
| | [dR](T)[sP].[dR](A)[sP].[dR](G)[sP].[LR](G)[PS2].[LR]([5meC]), |
| (SEQ ID NO 2, CMP ID NO 2_4) | |
| (h) | [LR](A)[PS2].[LR](A)[PS2].[dR](A)[sP].[mR](A)[sP].[LR](G)[sP].[dR](T)[sP].[dR](T)[sP]. |
| | [dR](T)[sP].[dR](C)[sP].[dR](T)[sP].[dR](C)[sP].[dR](A)[sP].[dR](G)[sP].[dR](A)[sP]. |
| | [dR](T)[sP].[dR](A)[sP].[mR](G)[sP].[LR](G)[PS2].[LR]([5meC]), |
| (SEQ ID NO 2, CMP ID NO 2_5) | |
| (i) | [LR](A)[PS2].[LR](A)[sP].[LR](A)[sP].[dR](A)[sP].[dR](G)[sP].[dR](T)[sP].[dR](T)[sP]. |
| | [dR](T)[sP].[dR](C)[sP].[dR](T)[sP].[dR](C)[sP].[mR](A)[sP].[dR](G)[sP].[dR](A)[sP]. |
| | [dR](T)[sP].[dR](A)[sP].[LR](G)[sP].[LR](G)[PS2].[LR]([5meC]), |
| (SEQ ID NO 2, CMP ID NO 2_6) | |
| (j) | [LR](A)[PS2].[MOE](A)[sP].[MOE](A)[sP].[dR](A)[sP].[dR](G)[sP].[dR](T)[sP].[dR](T) |
| | [sP].[dR](T)[sP].[dR](C)[sP].[dR](T)[sP].[dR](C)[sP].[mR](A)[sP].[dR](G)[sP].[dR](A) |
| | [sP].[dR](T)[sP].[dR](A)[sP].[LR](G)[PS2].[LR](G)[PS2].[MOE]([5meC]), |
| (SEQ ID NO 2, CMP ID NO 2_7) | |
| (k) | [LR](A)[PS2].[LR](A)[sP].[LR](A)[sP].[dR](A)[sP].[dR](G)[sP].[dR](T)[sP].[dR](T)[sP]. |
| | [dR](T)[sP].[dR](C)[sP].[dR](T)[sP].[mR](C)[sP].[dR](A)[sP].[dR](G)[sP].[dR](A)[sP]. |
| | [dR](T)[sP].[dR](A)[sP].[LR](G)[sP].[LR](G)[PS2].[LR]([5meC]), |
| (SEQ ID NO 2, CMP ID NO 2_8) | |
| (l) | [MOE](A)[sP].[MOE](A)[sP].[LR](A)[PS2].[dR](A)[sP].[dR](G)[sP].[dR](T)[sP].[dR](T) |
| | [sP].[dR](T)[sP].[dR](C)[sP].[dR](T)[sP].[mR](C)[sP].[dR](A)[sP].[dR](G)[sP].[dR](A) |
| | [sP].[dR](T)[sP].[dR](A)[sP].[LR](G)[sP].[LR](G)[PS2].[MOE]([5meC]), |
| (SEQ ID NO 2, CMP ID NO 2_9) | |
| (m) | [MOE](A)[sP].[LR](A)[sP].[LR](A)[sP].[dR](A)[sP].[dR](G)[sP].[dR](T)[sP].[dR](T)[sP]. |
| | [dR](T)[sP].[dR](C)[sP].[dR](T)[sP].[dR](C)[sP].[mR](A)[sP].[dR](G)[sP].[dR](A)[sP]. |
| | [dR](T)[sP].[dR](A)[sP].[LR](G)[sP].[LR](G)[sP].[MOE]([5meC]), |
| (SEQ ID NO 2, CMP ID NO 2_10) | |
| (n) | [LR](A)[PS2].[LR](A)[sP].[LR](A)[sP].[dR](A)[sP].[dR](G)[sP].[dR](T)[sP].[dR](T)[sP]. |
| | [dR](T)[sP].[mR](C)[sP].[dR](T)[sP].[dR](C)[sP].[dR](A)[sP].[dR](G)[sP].[dR](A)[sP]. |
| | [dR](T)[sP].[dR](A)[sP].[LR](G)[sP].[LR](G)[PS2].[LR]([5meC]), |
| (SEQ ID NO 2, CMP ID NO 2_11) | |
| (o) | [MOE](A)[sP].[LR](A)[sP].[LR](A)[sP].[dR](A)[sP].[dR](G)[sP].[dR](T)[sP].[dR](T)[sP]. |
| | [dR](T)[sP].[mR](C)[sP].[dR](T)[sP].[dR](C)[sP].[dR](A)[sP].[dR](G)[sP].[dR](A)[sP]. |
| | [dR](T)[sP].[dR](A)[sP].[LR](G)[sP].[LR](G)[PS2].[MOE]([5meC]), |
| (SEQ ID NO 2, CMP ID NO 2_12) | |
| (p) | [MOE](A)[sP].[LR](A)[PS2].[LR](A)[sP].[dR](A)[sP].[dR](G)[sP].[dR](T)[sP].[dR](T)[sP]. |
| | [dR](T)[sP].[dR](C)[sP].[dR](T)[sP].[dR](C)[sP].[dR](A)[sP].[dR](G)[sP].[dR](A)[sP]. |
| | [dR](T)[sP].[mR](A)[sP].[LR](G)[sP].[LR](G)[PS2].[MOE]([5meC]), |
| and | |
| (SEQ ID NO 18, CMP ID NO 17_1) | |
| (q) | [MOE](A)[sP].[MOE](A)[P].[LR](A)[sP].[dR](A)[sP].[dR](G)[sP].[dR](T)[sP].[dR](T) |
| | [sP].[mR](U)[sP].[dR](C)[sP].[dR](T)[sP].[dR](C)[sP].[dR](A)[sP].[dR](G)[sP].[dR](A) |
| | [sP].[dR](T)[sP].[dR](A)[sP].[LR](G)[sP].[LR](G)[PS2].[MOE]([5meC]), |

wherein
(A) is a nucleoside comprising an adenine nucleobase,
(C) is a nucleoside comprising a cytosine nucleobase,
([5meC]) is a nucleoside comprising a 5-methyl cytosine nucleobase,
([PPG]) is a nucleoside comprising a 7-deaza-8-aza guanine nucleobase,
(G) is a nucleoside comprising a guanine nucleobase,
(U) is a nucleoside comprising a uracil nucleobase,
(T) is a nucleoside comprising a thymine nucleobase,
[LR] indicates that the following nucleoside is a beta-D-oxy-LNA nucleoside,
[dR] indicates that the following nucleoside is a DNA nucleoside,
[mR] indicates that the following nucleoside is a 2′-O-methyl RNA nucleoside,
[MOE] indicates that the following nucleoside is a 2′-O-methoxyethyl RNA (MOE RNA) nucleoside,
[sP] indicates a phosphorothioate internucleoside linkage between the preceding and following nucleoside, and
[PS2] indicates a phosphorodithioate internucleoside linkage between the preceding and following nucleoside.

In some embodiments, the antisense oligonucleotide comprises or consists of a compound selected from the following:

(SEQ ID NO 2, CMP ID NO 2_1)

| | |
|---|---|
| (a) | [LR](A)[PS2].[LR](A)[sP].[dR](A)[sP].[mR](A) |
| | [sP].[LR](G)[sP].[dR](T)[sP].[dR](T)[sP]. |
| | [dR](T)[sP].[dR](C)[sP].[dR](T)[sP].[dR](C) |
| | [sP].[dR](A)[sP].[dR](G)[sP].[dR](A)[sP]. |
| | [dR](T)[sP].[dR](A)[sP].[mR](G)[sP].[LR](G) |
| | [PS2].[LR]([5meC]), |

and
(SEQ ID NO 2, CMP ID NO 2_2)

| | |
|---|---|
| (b) | [LR](A)[PS2].[LR](A)[PS2].[dR](A)[sP].[mR] |
| | (A)[sP].[LR](G)[sP].[dR](T)[sP].[dR](T)[sP]. |
| | [dR](T)[sP].[dR](C)[sP].[dR](T)[sP].[dR](C) |
| | [sP].[dR](A)[sP].[dR](G)[sP].[dR](A)[sP]. |
| | [dR](T)[sP].[dR](A)[sP].[dR](G)[sP].[LR](G) |
| | [PS2].[LR]([5meC]). |

In some embodiments, the antisense oligonucleotide comprises or consists of the following compound:

(SEQ ID NO 2, CMP ID NO 2_1)
[LR](A)[PS2].[LR](A)[sP].[dR](A)[sP].[mR](A)[sP].
[LR](G)[sP].[dR](T)[sP].[dR](T)[sP].[dR](T)[sP].
[dR](C)[sP].[dR](T)[sP].[dR](C)[sP].[dR](A)[sP].
[dR](G)[sP].[dR](A)[sP].[dR](T)[sP].[dR](A)[sP].
[mR](G)[sP].[LR](G)[PS2].[LR]([5meC]).

In some embodiments, the antisense oligonucleotide comprises the following compound:

(SEQ ID NO 2, CMP ID NO 2_1)
[LR](A)[PS2].[LR](A)[sP].[dR](A)[sP].[mR](A)
[sP].[LR](G)[sP].[dR](T)[sP].[dR](T)[sP].[dR]
(T)[sP].[dR](C)[sP].[dR](T)[sP].[dR](C)[sP].
[dR](A)[sP].[dR](G)[sP].[dR](A)[sP].[dR](T)
[sP].[dR](A)[sP].[mR](G)[sP].[LR](G)[PS2].
[LR]([5meC]).

In some embodiments, the antisense oligonucleotide consists of the following compound:

(SEQ ID NO 2, CMP ID NO 2_1)
[LR](A)[PS2].[LR](A)[sP].[dR](A)[sP].[mR](A)[sP].
[LR](G)[sP].[dR](T)[sP].[dR](T)[sP].[dR](T)[sP].
[dR](C)[sP].[dR](T)[sP].[dR](C)[sP].[dR](A)[sP].
[dR](G)[sP].[dR](A)[sP].[dR](T)[sP].[dR](A)[sP].
[mR](G)[sP].[LR](G)[PS2].[LR]([5meC]).

In some embodiments, the antisense oligonucleotide conjugate comprises or consists of any one of CNJ ID NOs 2_1, 2_2, 15_1, 16_1, 162, 163, 2_3, 2_4, 2_5, 2_6, 2_7, 2_8, 2_9, 2_10, 2_11, 2_12, and 17_1.

In some embodiments, the antisense oligonucleotide conjugate comprises or consists of any one of CNJ ID NOs 2_1, 2_2, 2_3, 2_4, 2_5, 2_6, 2_7, 2_8, 2_9, 210, 2_11, and 2_12.

In some embodiments, the antisense oligonucleotide conjugate comprises or consists of CNJ ID NO 2_1 or CNJ ID NO 2_2.

In some embodiments, the antisense oligonucleotide conjugate comprises or consists of CNJ ID NO 2_1.

The invention provides an antisense oligonucleotide conjugate selected from the following:

| (SEQ ID NO 2, CMP ID NO 2_1, CNJ ID NO 2_1) | |
|---|---|
| (a) | [5gn2c6]P.[dR](C)P.[dR](A)P.[LR](A)[PS2].[LR](A)[P].[dR](A)[sP].[mR](A)[sP].[LR] |
| | (G)[sP].[dR](T)[sP].[dR](T)[sP].[dR](T)[sP].[dR](C)[sP].[dR](T)[sP].[dR](C)[sP].[dR](A) |
| | [sP].[dR](G)[sP].[dR](A)[sP].[dR](T)[sP].[dR](A)[sP].[mR](G)[sP].[LR](G)[PS2].[LR] |
| | ([5meC]), |
| (SEQ ID NO 2, CMP ID NO 2_2, CNJ ID NO 2_2) | |
| (b) | [5gn2c6]P.[dR](C)P.[dR](A)P.[LR](A)[PS2].[LR](A)[PS2].[dR](A)[sP].[mR](A)[sP].[LR] |
| | (G)[sP].[dR](T)[sP].[dR](T)[sP].[dR](T)[sP].[dR](C)[sP].[dR](T)[sP].[dR](C)[sP].[dR](A) |
| | [sP].[dR](G)[sP].[dR](A)[sP].[dR](T)[sP].[dR](A)[sP].[dR](G)[sP].[LR](G)[PS2].[LR] |
| | ([5meC]), |
| (SEQ ID NO 15, CMP ID NO 15_1, CNJ ID NO 15_1) | |
| (c) | [5gn2c6]P.[dR](C)P.[dR](A)P.[LR](A)[PS2].[LR](A)[sP].[LR](A)[sP].[dR](A)[sP].[dR] |
| | ([PPG])[sP].[dR](T)[sP].[dR](T)[sP].[dR](T)[sP].[dR](C)[sP].[dR](T)[sP].[dR](C)[sP]. |
| | [dR](A)[sP].[dR](G)[sP].[dR](A)[sP].[dR](T)[sP].[dR](A)[sP].[LR](G)[sP].[LR](G)[PS2]. |
| | [LR]([5meC]), |
| (SEQ ID NO 16, CMP ID NO 16_1, CNJ ID NO 16_1) | |
| (d) | [5gn2c6]P.[dR](C)P.[dR](A)P.[LR](A)[PS2].[LR](A)[sP].[LR](A)[sP].[dR](A)[sP].[dR](G) |
| | [sP].[dR](T)[sP].[dR](T)[sP].[dR](T)[sP].[dR](C)[sP].[dR](T)[sP].[dR](C)[sP].[dR](A) |
| | [sP].[dR]([PPG])[sP].[dR](A)[sP].[dR](T)[sP].[dR](A)[sP].[LR](G)[sP].[LR](G)[PS2].[LR] |
| | ([5meC]), |
| (SEQ ID NO 16, CMP ID NO 16_2, CNJ ID NO 16_2) | |
| (e) | [5gn2c6]P.[dR](C)P.[dR](A)P.[MOE](A)[sP].[LR](A)[PS2].[LR](A)[sP].[dR](A)[sP].[dR] |
| | (G)[sP].[dR](T)[sP].[dR](T)[sP].[dR](T)[sP].[dR](C)[sP].[dR](T)[sP].[dR](C)[sP].[dR](A) |
| | [sP].[dR]([PPG])[sP].[dR](A)[sP].[dR](T)[sP].[dR](A)[sP].[LR](G)[sP].[LR](G)[PS2]. |
| | [MOE]([5meC]), |
| (SEQ ID NO 16, CMP ID NO 16_3, CNJ ID NO 16_3) | |
| (f) | [5gn2c6]P.[dR](C)P.[dR](A)P.[MOE](A)[sP].[LR](A)[PS2].[LR](A)[sP].[dR](A)[sP].[dR] |
| | (G)[sP].[dR](T)[sP].[dR](T)[sP].[dR](T)[sP].[dR](C)[sP].[dR](T)[sP].[dR](C)[sP].[dR](A) |
| | [sP].[dR]([PPG])[sP].[dR](A)[sP].[dR](T)[sP].[dR](A)[sP].[LR](G)[PS2].[LR](G)[PS2]. |
| | [MOE]([5meC]), |
| (SEQ ID NO 2, CMP ID NO 2_3, CNJ ID NO 2_3) | |
| (g) | [5gn2c6]P.[dR](C)P.[dR](A)P.[LR](A)[PS2].[LR](A)[sP].[dR](A)[sP].[mR](A)[sP].[LR] |
| | (G)[sP].[dR](T)[sP].[dR](T)[sP].[dR](T)[sP].[dR](C)[sP].[dR](T)[sP].[dR](C)[sP].[dR](A) |
| | [sP].[dR](G)[sP].[dR](A)[sP].[dR](T)[sP].[dR](A)[sP].[dR](G)[sP].[LR](G)[PS2].[LR] |
| | ([5meC]), |
| (SEQ ID NO 2, CMP ID NO 2_4, CNJ ID NO 2_4) | |
| (h) | [5gn2c6]P.[dR](C)P.[dR](A)P.[LR](A)[PS2].[LR](A)[PS2].[dR](A)[sP].[mR](A)[sP].[LR] |
| | (G)[sP].[dR](T)[sP].[dR](T)[sP].[dR](T)[sP].[dR](C)[sP].[dR](T)[sP].[dR](C)[sP].[dR](A) |
| | [sP].[dR](G)[sP].[dR](A)[sP].[dR](T)[sP].[dR](A)[sP].[mR](G)[sP].[LR](G)[PS2].[LR] |
| | ([5meC]), |
| (SEQ ID NO 2, CMP ID NO 2_5, CNJ ID NO 2_5) | |
| (i) | [5gn2c6]P.[dR](C)P.[dR](A)P.[LR](A)[PS2].[LR](A)[sP].[LR](A)[sP].[dR](A)[sP].[dR](G) |
| | [sP].[dR](T)[sP].[dR](T)[sP].[dR](T)[sP].[dR](C)[sP].[dR](T)[sP].[dR](C)[sP].[mR](A) |
| | [sP].[dR](G)[sP].[dR](A)[sP].[dR](T)[sP].[dR](A)[sP].[LR](G)[sP].[LR](G)[PS2].[LR] |
| | ([5meC]), |
| (SEQ ID NO 2, CMP ID NO 2_6, CNJ ID NO 2_6) | |
| (j) | [5gn2c6]P.[dR](C)P.[dR](A)P.[LR](A)[PS2].[MOE](A)[sP].[MOE](A)[sP].[dR](A)[sP]. |
| | [dR](G)[sP].[dR](T)[sP].[dR](T)[sP].[dR](T)[sP].[dR](C)[sP].[dR](T)[sP].[dR](C)[sP]. |
| | [mR](A)[sP].[dR](G)[sP].[dR](A)[P].[dR](T)[sP].[dR](A)[sP].[LR](G)[PS2].[LR](G) |
| | [PS2].[MOE]([5meC]), |
| (SEQ ID NO 2, CMP ID NO 2_7, CNJ ID NO 2_7) | |
| (k) | [5gn2c6]P.[dR](C)P.[dR](A)P.[LR](A)[PS2].[LR](A)[sP].[LR](A)[sP].[dR](A)[sP].[dR](G) |
| | [sP].[dR](T)[sP].[dR](T)[sP].[dR](T)[sP].[dR](C)[sP].[dR](T)[sP].[mR](C)[sP].[dR](A) |
| | [sP].[dR](G)[sP].[dR](A)[sP].[dR](T)[sP].[dR](A)[sP].[LR](G)[sP].[LR](G)[PS2].[LR] |
| | ([5meC]), |
| (SEQ ID NO 2, CMP ID NO 2_8, CNJ ID NO 2_8) | |
| (l) | [5gn2c6]P.[dR](C)P.[dR](A)P.[MOE](A)[sP].[MOE](A)[P].[LR](A)[PS2].[dR](A)[sP]. |
| | [dR](G)[sP].[dR](T)[sP].[dR](T)[sP].[dR](T)[sP].[dR](C)[sP].[dR](T)[sP].[mR](C)[sP]. |
| | [dR](A)[sP].[dR](G)[sP].[dR](A)[sP].[dR](T)[sP].[dR](A)[sP].[LR](G)[sP].[LR](G)[PS2]. |
| | [MOE]([5meC]), |
| (SEQ ID NO 2, CMP ID NO 2_9, CNJ ID NO 2_9) | |
| (m) | [5gn2c6]P.[dR](C)P.[dR](A)P.[MOE](A)[sP].[LR](A)[sP].[LR](A)[sP].[dR](A)[sP].[dR] |
| | (G)[sP].[dR](T)[sP].[dR](T)[sP].[dR](T)[sP].[dR](C)[sP].[dR](T)[sP].[dR](C)[sP].[mR] |
| | (A)[sP].[dR](G)[sP].[dR](A)[sP].[dR](T)[sP].[dR](A)[sP].[LR](G)[sP].[LR](G)[sP].[MOE] |
| | ([5meC]), |
| (SEQ ID NO 2, CMP ID NO 2_10, CNJ ID NO 2_10) | |
| (n) | [5gn2c6]P.[dR](C)P.[dR](A)P.[LR](A)[PS2].[LR](A)[sP].[LR](A)[sP].[dR](A)[sP].[dR](G) |
| | [sP].[dR](T)[sP].[dR](T)[sP].[dR](T)[sP].[mR](C)[sP].[dR](T)[sP].[dR](C)[sP].[dR](A) |
| | [sP].[dR](G)[sP].[dR](A)[sP].[dR](T)[sP].[dR](A)[sP].[LR](G)[sP].[LR](G)[PS2].[LR] |
| | ([5meC]), |
| (SEQ ID NO 2, CMP ID NO 2_11, CNJ ID NO 2_11) | |
| (o) | [5gn2c6]P.[dR](C)P.[dR](A)P.[MOE](A)[sP].[LR](A)[sP].[LR](A)[sP].[dR](A)[sP].[dR] |
| | (G)[sP].[dR](T)[sP].[dR](T)[sP].[dR](T)[sP].[mR](C)[sP].[dR](T)[sP].[dR](C)[sP].[dR] |
| | (A)[sP].[dR](G)[sP].[dR](A)[sP].[dR](T)[sP].[dR](A)[sP].[LR](G)[sP].[LR](G)[PS2]. |
| | [MOE]([5meC]), |
| (SEQ ID NO 2, CMP ID NO 2_12, CNJ ID NO 2_12) | |
| (p) | [5gn2c6]P.[dR](C)P.[dR](A)P.[MOE](A)[sP].[LR](A)[PS2].[LR](A)[sP].[dR](A)[sP].[dR] |
| | (G)[sP].[dR](T)[sP].[dR](T)[sP].[dR](T)[sP].[dR](C)[sP].[dR](T)[sP].[dR](C)[sP].[dR](A) |
| | [sP].[dR](G)[sP].[dR](A)[sP].[dR](T)[sP].[mR](A)[sP].[LR](G)[sP].[LR](G)[PS2].[MOE] |
| | ([5meC]), |
| and | |
| (SEQ ID NO 17, CMP ID NO 17_1, CNJ ID NO 17_1) | |
| (q) | [5gn2c6]P.[dR](C)P.[dR](A)P.[MOE](A)[sP].[MOE](A)[sP].[LR](A)[sP].[dR](A)[sP].[dR] |
| | (G)[sP].[dR](T)[sP].[dR](T)[sP].[mR](U)[sP].[dR](C)[sP].[dR](T)[sP].[dR](C)[sP].[dR] |
| | (A)[sP].[dR](G)[sP].[dR](A)[sP].[dR](T)[sP].[dR](A)[sP].[LR](G)[sP].[LR](G)[PS2]. |
| | [MOE]([5meC]), |

wherein
(A) is a nucleoside comprising an adenine nucleobase,
(C) is a nucleoside comprising a cytosine nucleobase,
([5meC]) is a nucleoside comprising a 5-methyl cytosine nucleobase,
([PPG]) is a nucleoside comprising a 7-deaza-8-aza guanine nucleobase,
(G) is a nucleoside comprising a guanine nucleobase,
(U) is a nucleoside comprising a uracil nucleobase,
(T) is a nucleoside comprising a thymine nucleobase,
[LR] indicates that the following nucleoside is a beta-D-oxy-LNA nucleoside,
[dR] indicates that the following nucleoside is a DNA nucleoside,
[mR] indicates that the following nucleoside is a 2′-O-methyl RNA nucleoside,
[MOE] indicates that the following nucleoside is a 2′-O-methoxyethyl RNA (MOE RNA) nucleoside,
[sP] indicates a phosphorothioate internucleoside linkage between the preceding and following nucleoside,
[PS2] indicates a phosphorodithioate internucleoside linkage between the preceding and following nucleoside,
P indicates a phosphodiester internucleoside linkage between the preceding nucleoside or moiety and the following nucleoside, and
[5gn2c6] is an aminohexyl conjugated tri(N-acetyl-galactosamine) moiety as shown in FIG. 8.

In some embodiments, the antisense oligonucleotide conjugate is selected from the following:

(SEQ ID NO 2, CMP ID NO 2_1, CNJ ID NO 2_1)

| | |
|---|---|
| (a) | [5gn2c6]P.[dR](C)P.[dR](A)P.[LR](A)[PS2].[LR] |
| | (A)[sP].[dR](A)[sP].[mR](A)[sP].[LR](G)[sP]. |
| | [dR](T)[sP].[dR](T)[sP].[dR](T)[sP].[dR](C) |
| | [sP].[dR](T)[sP].[dR](C)[sP].[dR](A)[sP].[dR] |
| | (G)[sP].[dR](A)[P].[dR](T)[sP].[dR](A)[sP]. |
| | [mR](G)[sP].[LR](G)[PS2].[LR]([5meC]), |

and
(SEQ ID NO 2, CMP ID NO 2_2, CNJ ID NO 2_2)

| | |
|---|---|
| (b) | [5gn2c6]P.[dR](C)P.[dR](A)P.[LR](A)[PS2].[LR] |
| | (A)[PS2].[dR](A)[sP].[mR](A)[sP].[LR](G)[sP]. |
| | [dR](T)[sP].[dR](T)[sP].[dR](T)[sP].[dR](C) |
| | [sP].[dR](T)[sP].[dR](C)[sP].[dR](A)[sP].[dR] |
| | (G)[sP].[dR](A)[sP].[dR](T)[sP].[dR](A)[sP]. |
| | [dR](G)[sP].[LR](G)[PS2].[LR]([5meC]). |

In some embodiments, the antisense oligonucleotide conjugate is the following:

(SEQ ID NO 2, CMP ID NO 2_1, CNJ ID NO 2_1)
[5gn2c6]P.[dR](C)P.[dR](A)P.[LR](A)[PS2].[LR](A)
[sP].[dR](A)[sP].[mR](A)[sP].[LR](G)[sP].[dR](T)
[sP].[dR](T)[sP].[dR](T)[sP].[dR](C)[sP].[dR](T)
[sP].[dR](C)[sP].[dR](A)[sP].[dR](G)[sP].[dR](A)
[sP].[dR](T)[P].[dR](A)[P].[mR](G)[sP].[LR](G)
[PS2].[LR]([5meC]).

The invention provides an antisense oligonucleotide conjugate which is CNJ ID NO 2_1 as depicted in FIG. 2.

Targeting A1CF 6951-6970

The antisense oligonucleotide or antisense oligonucleotide conjugate of the invention may bind a target sequence within the region defined by positions 6951-6970 of the A1CF pre-mRNA (SEQ ID NO 45).

Thus, the invention provides an antisense oligonucleotide of 12 to 30 nucleotides in length, which comprises a contiguous nucleotide sequence of 12 to 30 nucleotides in length, wherein the contiguous nucleotide sequence is capable of binding to a target sequence in an A1CF mRNA, wherein the target sequence is a sequence of at least 17 contiguous nucleotides within GAGAAAAACCUAUAAUGCCU (SEQ ID NO 42) (positions 6951-6970 of SEQ ID NO 45), and wherein the antisense oligonucleotide is capable of reducing A1CF expression.

The invention also provides an antisense oligonucleotide conjugate comprising an antisense oligonucleotide covalently attached to at least one conjugate moiety, wherein the antisense oligonucleotide is an antisense oligonucleotide of 12 to 30 nucleotides in length, which comprises a contiguous nucleotide sequence of 12 to 30 nucleotides in length, wherein the contiguous nucleotide sequence is capable of binding to a target sequence in an A1CF mRNA, wherein the target sequence is a sequence of at least 17 contiguous nucleotides within GAGAAAAACCUAUAAUGCCU (SEQ ID NO 42) (positions 6951-6970 of SEQ ID NO 45), and wherein the antisense oligonucleotide is capable of reducing A1CF expression.

In some embodiments of the antisense oligonucleotide or the antisense oligonucleotide conjugate of the invention, the target sequence is a sequence of 17, 18, 19 or 20 contiguous nucleotides within SEQ ID NO 42.

In some embodiments, the target sequence is selected from the following sequences:

(SEQ ID NO 31)
GAAAAACCUAUAAUGCCU (positions 6953-6970 of SEQ ID
NO 45),
(SEQ ID NO 36)
AGAAAAACCUAUAAUGC (positions 6952-6968 of SEQ ID
NO 45),
(SEQ ID NO 37)
GAAAAACCUAUAAUGCC (positions 6953-6969 of SEQ ID
NO 45),
and
(SEQ ID NO 41)
GAGAAAAACCUAUAAUGC (positions 6951-6968 of SEQ ID
NO 45).

In some embodiments, the target sequence is selected from SEQ ID NOs 31, 36 and 37

In some embodiments, the target sequence is SEQ ID NO 31.

In some embodiments, the sequence of the antisense oligonucleotide and/or the sequence of the contiguous nucleotide sequence comprises or consists of a sequence having at least 80% identity to any one of the following sequences:

(SEQ ID NO 1)
AGGCATTATAGGTTTTTE,
(SEQ ID NO 6)
GEATTATAGGTTTTTET,
(SEQ ID NO 7)
GGCATTATAGGTTTTTE,
(SEQ ID NO 8)
GGEATTATAGGTTTTTE,
(SEQ ID NO 18)
AGGCATTATAGGUTTTTE,
(SEQ ID NO 19)
AGGCAUTATAGGTTTTTE,
(SEQ ID NO 20)
GEATTATAGGTTTTTCTE,
(SEQ ID NO 21)
GEATTATAGGTTTTUCTE,
(SEQ ID NO 22)
GEATTATAGGTTTUTCTE,
(SEQ ID NO 23)
GEATTATAGGTTUTTCTE,
(SEQ ID NO 24)
GEATTATAGGTTUTTET,
(SEQ ID NO 25)
GEATTATAGGTUTTTET,
(SEQ ID NO 26)
GEATTATAGGUTTTTCTE,
(SEQ ID NO 27)
GEATTATAGGUTTTTET,
(SEQ ID NO 28)
GEATTAUAGGTTTTTET,
(SEQ ID NO 29)
GEATUATAGGTTTTTET
and
(SEQ ID NO 30)
GEAUTATAGGTTTTTET.

In some embodiments, the sequence of the antisense oligonucleotide and/or the sequence of the contiguous nucleotide sequence comprises or consists of a sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to any one of SEQ ID NOs 1, 6, 7, 8, 18, 19, 20, 21, 22, 23, 24, 25, 26, 27, 28, 29 or 30.

In some embodiments, the sequence of the antisense oligonucleotide and/or the sequence of the contiguous nucleotide sequence comprises or consists of a sequence having at least 80% identity to any one of SEQ ID NOs 1, 6, 7 or 8.

In some embodiments, the antisense oligonucleotide and/or the sequence of the contiguous nucleotide sequence comprises or consists of a sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to any one of SEQ ID NOs 1, 6, 7 or 8.

In some embodiments, the sequence of the antisense oligonucleotide and/or the sequence of the contiguous nucleotide sequence comprises or consists of a sequence having at least 80% identity to SEQ ID NO 1.

In some embodiments, the sequence of the antisense oligonucleotide and/or the sequence of the contiguous nucleotide sequence comprises or consists of a sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO 1.

In some embodiments, the sequence of the antisense oligonucleotide and/or the sequence of the contiguous nucleotide sequence consists of SEQ ID NO 1. In some embodiments, the sequence of the contiguous nucleotide sequence consists of SEQ ID NO 1. In some embodiments, the sequence of the antisense oligonucleotide consists of SEQ ID NO 1.

In some embodiments, the antisense oligonucleotide comprises or consists of any one of CMP ID NOs 1_1, 6_1, 7_1, 7_2, 8_1, 18_1, 19_1, 20_1, 202, 203, 204, 205, 206, 6_2, 6_3, 6_4, 6_5, 6_6, 6_7, 6_8, 6_9, 6_10, 6_11, 6_12, 21_1, 22_1, 23_1, 24_1, 25_1, 25_2, 26_1, 27_1, 27_2, 28_1, 29_1, and 30_1.

In some embodiments, the antisense oligonucleotide comprises or consists of any one of CMP ID NOs 1_1, 6_1, 7_1, 7_2, and 8_1.

In some embodiments, the antisense oligonucleotide comprises CMP ID NO 1_1. In some embodiments, the antisense oligonucleotide consists of CMP ID NO 1_1.

The invention provides an antisense oligonucleotide comprising or consisting of a compound selected from the following:

| (SEQ ID NO 1, CMP ID NO 1_1) | |
|---|---|
| (a) | [LR](A)[sP].[LR](G)[sP].[dR](G)[sP].[dR](C)[sP].[LR](A)[sP].[dR](T)[sP].[dR](T)[sP]. |
| | [dR](A)[sP].[dR](T)[sP].[dR](A)[sP].[dR](G)[sP].[dR](G)[sP].[dR](T)[sP].[dR](T)[sP]. |
| | [dR](T)[sP].[LR](T)[sP].[LR](T)[sP].[LR]([5meC]), |
| (SEQ ID NO 6, CMP ID NO 6_1) | |
| (b) | [LR](G)[sP].[LR]([5meC])[sP].[dR](A)[sP].[dR](T)[P].[dR](T)[sP].[dR](A)[sP].[dR](T) |
| | [sP].[dR](A)[sP].[dR](G)[sP].[dR](G)[sP].[dR](T)[sP].[dR](T)[sP].[dR](T)[sP].[LR](T) |
| | [sP].[LR](T)[sP].[LR]([5meC])[sP].[LR](T), |
| (SEQ ID NO 7, CMP ID NO 7_1) | |
| (c) | [MOE](G)[sP].[LR](G)[sP].[dR](C)[sP].[LR](A)[sP].[dR](T)[sP].[dR](T)[sP].[dR](A)[sP]. |
| | [dR](T)[sP].[dR](A)[sP].[dR](G)[sP].[dR](G)[sP].[dR](T)[sP].[dR](T)[sP].[LR](T)[sP]. |
| | [MOE](T)[sP].[MOE](T)[sP].[MOE]([5meC]), |
| (SEQ ID NO 7, CMP ID NO 7_2) | |
| (d) | [LR](G)[sP].[LR](G)[sP].[dR](C)[sP].[LR](A)[sP].[dR](T)[sP].[dR](T)[sP].[dR](A)[sP]. |
| | [dR](T)[sP].[mR](A)[sP].[dR](G)[sP].[dR](G)[sP].[dR](T)[sP].[dR](T)[sP].[dR](T)[sP]. |
| | [LR](T)[sP].[LR](T)[sP].[LR]([5meC]), |
| (SEQ ID NO 8, CMP ID NO 8_1) | |
| (e) | [MOE](G)[sP].[MOE](G)[sP].[MOE]([5meC])[sP].[LR](A)[PS2].[dR](T)[sP].[dR](T)[sP]. |
| | [dR](A)[sP].[dR](T)[sP].[mR](A)[sP].[dR](G)[sP].[dR](G)[sP].[dR](T)[sP].[dR](T)[sP]. |
| | [LR](T)[PS2].[LR](T)[PS2].[LR](T)[PS2].[MOE]([5meC]), |
| (SEQ ID NO 17, CMP ID NO 17_1) | |
| (f) | [MOE](A)[sP].[MOE](A)[sP].[LR](A)[sP].[dR](A)[sP].[dR](G)[sP].[dR](T)[sP].[dR](T) |
| | [sP].[mR](U)[sP].[dR](C)[sP].[dR](T)[sP].[dR](C)[sP].[dR](A)[sP].[dR](G)[sP].[dR](A) |
| | [sP].[dR](T)[sP].[dR](A)[sP].[LR](G)[sP].[LR](G)[PS2].[MOE]([5meC]), |
| (SEQ ID NO 18, CMP ID NO 18_1) | |
| (g) | [LR](A)[PS2].[LR](G)[sP].[dR](G)[sP].[dR](C)[sP].[LR](A)[sP].[dR](T)[sP].[dR](T)[sP]. |
| | [dR](A)[sP].[dR](T)[sP].[dR](A)[sP].[dR](G)[sP].[dR](G)[sP].[mR](U)[sP].[dR](T)[sP]. |
| | [dR](T)[sP].[LR](T)[sP].[LR](T)[PS2].[LR]([5meC]), |
| (SEQ ID NO 19, CMP ID NO 19_1) | |
| (h) | [LR](A)[PS2].[LR](G)[sP].[dR](G)[sP].[dR](C)[sP].[LR](A)[sP].[mR](U)[sP].[dR](T)[sP]. |
| | [dR](A)[sP].[dR](T)[sP].[dR](A)[sP].[dR](G)[sP].[dR](G)[sP].[dR](T)[sP].[dR](T)[sP]. |
| | [dR](T)[sP].[LR](T)[sP].[LR](T)[PS2].[LR]([5meC]), |
| (SEQ ID NO 20, CMP ID NO 20_1) | |
| (i) | [LR](G)[PS2].[LR]([5meC])[sP].[dR](A)[sP].[dR](T)[sP].[dR](T)[sP].[dR](A)[sP].[dR](T) |
| | [sP].[dR](A)[sP].[dR](G)[sP].[dR](G)[sP].[dR](T)[sP].[LR](T)[sP].[dR](T)[sP].[dR](T) |
| | [sP].[dR](T)[sP].[dR](C)[sP].[LR](T)[PS2].[LR]([5meC]), |
| (SEQ ID NO 20, CMP ID NO 20_2) | |
| (j) | [LR](G)[PS2].[LR]([5meC])[sP].[mR](A)[sP].[dR](T)[sP].[dR](T)[sP].[dR](A)[sP].[dR](T) |
| | [sP].[dR](A)[sP].[dR](G)[sP].[dR](G)[sP].[dR](T)[sP].[LR](T)[sP].[dR](T)[sP].[dR](T) |
| | [sP].[dR](T)[sP].[dR](C)[sP].[LR](T)[PS2].[LR]([5meC]), |
| (SEQ ID NO 20, CMP ID NO 20_3) | |
| (k) | [LR](G)[PS2].[LR]([5meC])[sP].[dR](A)[sP].[dR](T)[sP].[dR](T)[sP].[dR](A)[sP].[dR](T) |
| | [sP].[mR](A)[P].[dR](G)[sP].[dR](G)[sP].[dR](T)[sP].[LR](T)[sP].[dR](T)[sP].[dR](T) |
| | [sP].[dR](T)[sP].[dR](C)[sP].[LR](T)[PS2].[LR]([5meC]), |
| (SEQ ID NO 20, CMP ID NO 20_4) | |
| (l) | [LR](G)[PS2].[LR]([5meC])[sP].[dR](A)[sP].[dR](T)[sP].[dR](T)[sP].[dR](A)[sP].[dR](T) |
| | [sP].[dR](A)[sP].[mR](G)[sP].[dR](G)[sP].[dR](T)[sP].[LR](T)[sP].[dR](T)[sP].[dR](T) |
| | [sP].[dR](T)[sP].[dR](C)[sP].[LR](T)[PS2].[LR]([5meC]), |
| (SEQ ID NO 20, CMP ID NO 20_5) | |
| (m) | [LR](G)[PS2].[LR]([5meC])[sP].[dR](A)[sP].[dR](T)[sP].[dR](T)[sP].[dR](A)[sP].[dR](T) |
| | [sP].[dR](A)[sP].[dR](G)[sP].[dR](G)[sP].[dR](T)[sP].[LR](T)[sP].[dR](T)[sP].[dR](T) |
| | [sP].[dR](T)[sP].[dR](C)[sP].[LR](T)[PS2].[MOE]([5meC]), |
| (SEQ ID NO 20, CMP ID NO 20_6) | |
| (aq) | [LR](G)[PS2].[LR]([5meC])[sP].[dR](A)[sP].[dR](T)[sP].[dR](T)[sP].[dR](A)[sP].[dR](T) |
| | [sP].[dR](A)[sP].[dR](G)[sP].[mR](G)[sP].[dR](T)[sP].[LR](T)[sP].[dR](T)[sP].[dR](T) |
| | [sP].[dR](T)[sP].[dR](C)[sP].[LR](T)[PS2].[LR]([5meC]), |
| (SEQ ID NO 6, CMP ID NO 6_2) | |
| (n) | [LR](G)[PS2].[LR]([5meC])[sP].[LR](A)[sP].[dR](T)[sP].[dR](T)[sP].[dR](A)[sP].[dR](T) |
| | [sP].[dR](A)[sP].[dR](G)[sP].[dR](G)[sP].[dR](T)[sP].[dR](T)[sP].[dR](T)[sP].[LR](T) |
| | [sP].[dR](T)[sP].[LR]([5meC])[PS2].[LR](T), |
| (SEQ ID NO 6, CMP ID NO 6_3) | |
| (o) | [LR](G)[PS2].[LR]([5meC])[sP].[LR](A)[sP].[dR](T)[sP].[dR](T)[sP].[dR](A)[sP].[dR](T) |
| | [sP].[mR](A)[sP].[dR](G)[sP].[dR](G)[sP].[dR](T)[sP].[dR](T)[sP].[dR](T)[sP].[LR](T) |
| | [sP].[dR](T)[sP].[LR]([5meC])[PS2].[LR](T), |
| (SEQ ID NO 6, CMP ID NO 6_4) | |
| (p) | [LR](G)[PS2].[LR]([5meC])[sP].[LR](A)[sP].[dR](T)[sP].[dR](T)[sP].[dR](A)[sP].[dR](T) |
| | [sP].[dR](A)[sP].[mR](G)[sP].[dR](G)[sP].[dR](T)[sP].[dR](T)[sP].[dR](T)[sP].[LR](T) |
| | [sP].[dR](T)[sP].[LR]([5meC])[PS2].[LR](T), |
| (SEQ ID NO 6, CMP ID NO 6_5) | |
| (q) | [LR](G)[PS2].[LR]([5meC])[sP].[mR](A)[sP].[dR](T)[sP].[dR](T)[sP].[dR](A)[sP].[dR](T) |
| | [sP].[dR](A)[sP].[dR](G)[sP].[dR](G)[sP].[dR](T)[sP].[dR](T)[sP].[dR](T)[sP].[LR](T) |
| | [sP].[LR](T)[sP].[LR]([5meC])[PS2].[LR](T), |
| (SEQ ID NO 6, CMP ID NO 6_6) | |
| (r) | [LR](G)[PS2].[LR]([5meC])[sP].[dR](A)[sP].[dR](T)[sP].[dR](T)[sP].[dR](A)[sP].[dR](T) |
| | [sP].[mR](A)[sP].[dR](G)[sP].[dR](G)[sP].[dR](T)[sP].[dR](T)[sP].[dR](T)[sP].[LR](T) |
| | [sP].[LR](T)[sP].[LR]([5meC])[PS2].[LR](T), |
| (SEQ ID NO 6, CMP ID NO 6_7) | |
| (s) | [LR](G)[PS2].[LR]([5meC])[sP].[dR](A)[sP].[dR](T)[sP].[dR](T)[sP].[dR](A)[sP].[dR](T) |
| | [sP].[dR](A)[sP].[mR](G)[sP].[dR](G)[sP].[dR](T)[sP].[dR](T)[sP].[dR](T)[sP].[LR](T) |
| | [sP].[LR](T)[sP].[LR]([5meC])[PS2].[LR](T), |
| (SEQ ID NO 6, CMP ID NO 6_8) | |
| (t) | [LR](G)[PS2].[LR]([5meC])[sP].[dR](A)[sP].[dR](T)[sP].[dR](T)[sP].[dR](A)[sP].[dR](T) |
| | [sP].[dR](A)[sP].[dR](G)[sP].[mR](G)[sP].[dR](T)[sP].[dR](T)[sP].[dR](T)[sP].[LR](T) |
| | [sP].[LR](T)[sP].[LR]([5meC])[PS2].[LR](T), |
| (SEQ ID NO 6, CMP ID NO 6_9) | |
| (u) | [MOE](G)[sP].[LR]([5meC])[PS2].[dR](A)[sP].[dR](T)[sP].[dR](T)[sP].[dR](A)[sP].[dR] |
| | (T)[sP].[dR](A)[sP].[dR](G)[sP].[dR](G)[sP].[dR](T)[sP].[dR](T)[sP].[dR](T)[sP].[LR](T) |
| | [sP].[LR](T)[sP].[LR]([5meC])[PS2].[MOE](T), |
| (SEQ ID NO 6, CMP ID NO 6_10) | |
| (v) | [MOE](G)[sP].[LR]([5meC])[PS2].[dR](A)[sP].[dR](T)[sP].[dR](T)[sP].[dR](A)[sP].[dR] |
| | (T)[sP].[dR](A)[sP].[dR](G)[sP].[dR](G)[sP].[dR](T)[sP].[dR](T)[sP].[dR](T)[sP].[LR](T) |
| | [sP].[LR](T)[PS2].[MOE]([5meC])[sP].[MOE](T), |
| (SEQ ID NO 6, CMP ID NO 6_11) | |
| (w) | [MOE](G)[sP].[LR]([5meC])[PS2].[dR](A)[sP].[dR](T)[sP].[dR](T)[sP].[dR](A)[sP].[dR] |
| | (T)[sP].[dR](A)[sP].[dR](G)[sP].[dR](G)[sP].[dR](T)[sP].[dR](T)[sP].[dR](T)[sP].[LR](T) |
| | [sP].[LR](T)[PS2].[LR]([5meC])[PS2].[MOE](T), |
| (SEQ ID NO 6, CMP ID NO 6_12) | |
| (x) | [MOE](G)[sP].[LR]([5meC])[PS2].[dR](A)[sP].[dR](T)[sP].[dR](T)[sP].[dR](A)[sP].[dR] |
| | (T)[sP].[dR](A)[sP].[dR](G)[sP].[dR](G)[sP].[dR](T)[sP].[dR](T)[sP].[dR](T)[sP].[LR](T) |
| | [PS2].[LR](T)[PS2].[MOE]([5meC])[sP].[MOE](T), |
| (SEQ ID NO 21, CMP ID NO 21_1) | |
| (y) | [LR](G)[PS2].[LR]([5meC])[sP].[dR](A)[sP].[dR](T)[sP].[dR](T)[sP].[dR](A)[sP].[dR](T) |
| | [sP].[dR](A)[sP].[dR](G)[sP].[dR](G)[sP].[dR](T)[sP].[LR](T)[sP].[dR](T)[P].[dR](T) |
| | [sP].[mR](U)[sP].[dR](C)[sP].[LR](T)[PS2].[LR]([5meC]), |
| (SEQ ID NO 22, CMP ID NO 22_1) | |
| (z) | [LR](G)[PS2].[LR]([5meC])[sP].[dR](A)[sP].[dR](T)[sP].[dR](T)[sP].[dR](A)[sP].[dR](T) |
| | [sP].[dR](A)[sP].[dR](G)[sP].[dR](G)[sP].[dR](T)[sP].[LR](T)[sP].[dR](T)[sP].[mR](U) |
| | [sP].[dR](T)[sP].[dR](C)[sP].[LR](T)[PS2].[LR]([5meC]), |
| (SEQ ID NO 23, CMP ID NO 23_1) | |
| (aa) | [LR](G)[PS2].[LR]([5meC])[sP].[dR](A)[sP].[dR](T)[sP].[dR](T)[sP].[dR](A)[sP].[dR](T) |
| | [sP].[dR](A)[sP].[dR](G)[sP].[dR](G)[sP].[dR](T)[sP].[LR](T)[sP].[mR](U)[sP].[dR](T) |
| | [sP].[dR](T)[sP].[dR](C)[sP].[LR](T)[PS2].[LR]([5meC]), |
| (SEQ ID NO 24, CMP ID NO 24_1) | |
| (ab) | [LR](G)[PS2].[LR]([5meC])[sP].[dR](A)[sP].[dR](T)[sP].[dR](T)[sP].[dR](A)[sP].[dR](T) |
| | [sP].[dR](A)[sP].[dR](G)[sP].[dR](G)[sP].[dR](T)[sP].[dR](T)[sP].[mR](U)[sP].[LR](T) |
| | [sP].[LR](T)[sP].[LR]([5meC])[PS2].[LR](T), |
| (SEQ ID NO 25, CMP ID NO 25_1) | |
| (ac) | [LR](G)[PS2].[LR]([5meC])[sP].[LR](A)[sP].[dR](T)[sP].[dR](T)[sP].[dR](A)[sP].[dR](T) |
| | [sP].[dR](A)[sP].[dR](G)[sP].[dR](G)[sP].[dR](T)[sP].[mR](U)[sP].[dR](T)[sP].[LR](T) |
| | [sP].[dR](T)[sP].[LR]([5meC])[PS2].[LR](T), |
| (SEQ ID NO 25, CMP ID NO 25_2) | |
| (bh) | [LR](G)[PS2].[LR]([5meC])[sP].[dR](A)[sP].[dR](T)[sP].[dR](T)[sP].[dR](A)[sP].[dR](T) |
| | [sP].[dR](A)[sP].[dR](G)[sP].[dR](G)[sP].[dR](T)[sP].[mR](U)[sP].[dR](T)[sP].[LR](T) |
| | [sP].[LR](T)[sP].[LR]([5meC])[PS2].[LR](T), |
| (SEQ ID NO 26, CMP ID NO 26_1) | |
| (ad) | [LR](G)[PS2].[LR]([5meC])[sP].[dR](A)[sP].[dR](T)[sP].[dR](T)[sP].[dR](A)[sP].[dR](T) |
| | [sP].[dR](A)[sP].[dR](G)[sP].[dR](G)[sP].[mR](U)[sP].[LR](T)[sP].[dR](T)[sP].[dR](T) |
| | [sP].[dR](T)[sP].[dR](C)[sP].[LR](T)[PS2].[LR]([5meC]), |
| (SEQ ID NO 27, CMP ID NO 27_1) | |
| (ae) | [LR](G)[PS2].[LR]([5meC])[sP].[LR](A)[sP].[dR](T)[sP].[dR](T)[sP].[dR](A)[sP].[dR](T) |
| | [sP].[dR](A)[sP].[dR](G)[sP].[dR](G)[sP].[mR](U)[sP].[dR](T)[sP].[dR](T)[sP].[LR](T) |
| | [sP].[dR](T)[sP].[LR]([5meC])[PS2].[LR](T), |
| (SEQ ID NO 27, CMP ID NO 27_2) | |
| (af) | [LR](G)[PS2].[LR]([5meC])[sP].[dR](A)[sP].[dR](T)[sP].[dR](T)[sP].[dR](A)[sP].[dR](T) |
| | [sP].[dR](A)[sP].[dR](G)[sP].[dR](G)[sP].[mR](U)[sP].[dR](T)[sP].[dR](T)[sP].[LR](T) |
| | [sP].[LR](T)[sP].[LR]([5meC])[PS2].[LR](T), |
| (SEQ ID NO 28, CMP ID NO 28_1) | |
| (ag) | [LR](G)[PS2].[LR]([5meC])[sP].[dR](A)[sP].[dR](T)[sP].[dR](T)[sP].[dR](A)[sP].[mR] |
| | (U)[sP].[dR](A)[sP].[dR](G)[sP].[dR](G)[sP].[dR](T)[sP].[dR](T)[sP].[dR](T)[sP].[LR](T) |
| | [sP].[LR](T)[sP].[LR]([5meC])[PS2].[LR](T), |
| (SEQ ID NO 29, CMP ID NO 29_1) | |
| (ah) | [LR](G)[PS2].[LR]([5meC])[sP].[dR](A)[sP].[dR](T)[sP].[mR](U)[sP].[dR](A)[sP].[dR] |
| | (T)[sP].[dR](A)[sP].[dR](G)[sP].[dR](G)[sP].[dR](T)[sP].[dR](T)[sP].[dR](T)[sP].[LR](T) |
| | [sP].[LR](T)[sP].[LR]([5meC])[PS2].[LR](T), |
| (SEQ ID NO 30, CMP ID NO 30_1) | |
| (ai) | [LR](G)[PS2].[LR]([5meC])[sP].[dR](A)[sP].[mR](U)[sP].[dR](T)[sP].[dR](A)[sP].[dR] |
| | (T)[sP].[dR](A)[sP].[dR](G)[sP].[dR](G)[sP].[dR](T)[sP].[dR](T)[sP].[dR](T)[sP].[LR](T) |
| | [sP].[LR](T)[sP].[LR]([5meC])[PS2].[LR](T), |

wherein
(A) is a nucleoside comprising an adenine nucleobase,
(C) is a nucleoside comprising a cytosine nucleobase,
([5meC]) is a nucleoside comprising a 5-methyl cytosine nucleobase,
([PPG]) is a nucleoside comprising a 7-deaza-8-aza guanine nucleobase,
(G) is a nucleoside comprising a guanine nucleobase,
(U) is a nucleoside comprising a uracil nucleobase,
(T) is a nucleoside comprising a thymine nucleobase,
[LR] indicates that the following nucleoside is a beta-D-oxy-LNA nucleoside,
[dR] indicates that the following nucleoside is a DNA nucleoside,
[mR] indicates that the following nucleoside is a 2′-O-methyl RNA nucleoside,
[MOE] indicates that the following nucleoside is a 2′-O-methoxyethyl RNA (MOE RNA) nucleoside,
[sP] indicates a phosphorothioate internucleoside linkage between the preceding and following nucleoside, and
[PS2] indicates a phosphorodithioate internucleoside linkage between the preceding and following nucleoside.

In some embodiments, the antisense oligonucleotide comprises or consists of a compound selected from the following:

| (SEQ ID NO 1, CMP ID NO 1_1) | |
|---|---|
| (a) | [LR](A)[sP].[LR](G)[sP].[dR](G)[sP].[dR](C)[sP].[LR](A)[sP].[dR](T)[sP].[dR](T)[sP]. |
| | [dR](A)[sP].[dR](T)[sP].[dR](A)[sP].[dR](G)[sP].[dR](G)[sP].[dR](T)[sP].[dR](T)[sP]. |
| | [dR](T)[sP].[LR](T)[sP].[LR](T)[sP].[LR]([5meC]), |
| (SEQ ID NO 6, CMP ID NO 6_1) | |
| (b) | [LR](G)[sP].[LR]([5meC])[sP].[dR](A)[sP].[dR](T)[sP].[dR](T)[sP].[dR](A)[sP].[dR](T) |
| | [sP].[dR](A)[sP].[dR](G)[sP].[dR](G)[sP].[dR](T)[sP].[dR](T)[sP].[dR](T)[sP].[LR](T) |
| | [sP].[LR](T)[sP].[LR]([5meC])[sP].[LR](T), |
| (SEQ ID NO 7, CMP ID NO 7_1) | |
| (c) | [MOE](G)[sP].[LR](G)[sP].[dR](C)[sP].[LR](A)[sP].[dR](T)[sP].[dR](T)[sP].[dR](A)[sP]. |
| | [dR](T)[sP].[dR](A)[sP].[dR](G)[sP].[dR](G)[sP].[dR](T)[sP].[dR](T)[sP].[LR](T)[sP]. |
| | [MOE](T)[sP].[MOE](T)[sP].[MOE]([5meC]), |
| (SEQ ID NO 7, CMP ID NO 7_2) | |
| (d) | [LR](G)[sP].[LR](G)[sP].[dR](C)[sP].[LR](A)[sP].[dR](T)[sP].[dR](T)[sP].[dR](A)[sP]. |
| | [dR](T)[sP].[mR](A)[sP].[dR](G)[sP].[dR](G)[sP].[dR](T)[sP].[dR](T)[sP].[dR](T)[sP]. |
| | [LR](T)[sP].[LR](T)[sP].[LR]([5meC]), |
| and | |
| (SEQ ID NO 8, CMP ID NO 8_1) | |
| (e) | [MOE](G)[sP].[MOE](G)[sP].[MOE]([5meC])[sP].[LR](A)[PS2].[dR](T)[sP].[dR](T)[sP]. |
| | [dR](A)[sP].[dR](T)[sP].[mR](A)[sP].[dR](G)[sP].[dR](G)[sP].[dR](T)[sP].[dR](T)[sP]. |
| | [LR](T)[PS2].[LR](T)[PS2].[LR](T)[PS2].[MOE]([5meC]). |

In some embodiments, the antisense oligonucleotide comprises the following compound:

(SEQ ID NO 1, CMP ID NO 1_1)
[LR](A)[sP].[LR](G)[sP].[dR](G)[sP].[dR](C)[sP].
[LR](A)[sP].[dR](T)[sP].[dR](T)[sP].[dR](A)[sP].
[dR](T)[sP].[dR](A)[sP].[dR](G)[sP].[dR](G)[sP].
[dR](T)[sP].[dR](T)[sP].[dR](T)[sP].[LR](T)[sP].
[LR](T)[sP].[LR]([5meC]).

In some embodiments, the antisense oligonucleotide consists of the following compound:

(SEQ ID NO 1, CMP ID NO 1_1)
[LR](A)[sP].[LR](G)[sP].[dR](G)[sP].[dR](C)[sP].
[LR](A)[sP].[dR](T)[sP].[dR](T)[sP].[dR](A)[sP].
[dR](T)[sP].[dR](A)[sP].[dR](G)[sP].[dR](G)[sP].
[dR](T)[sP].[dR](T)[sP].[dR](T)[sP].[LR](T)[sP].
[LR](T)[sP].[LR]([5meC]).

In some embodiments, the antisense oligonucleotide conjugate comprises or consists of any one of CNJ ID NOs 1_1, 6_1, 7_1, 7_2, 8_1, 18_1, 19_1, 20_1, 20_2, 20_3, 20_4, 20_5, 20_6, 6_2, 6_3, 6_4, 6_5, 6_6, 6_7, 6_8, 6_9, 6_10, 6_11, 6_12, 21_1, 22_1, 23_1, 24_1, 25_1, 25_2, 26_1, 27_1, 27_2, 28_1, 29_1, and 30_1.

In some embodiments, the antisense oligonucleotide conjugate comprises or consists of any one of CNJ ID NOs 1_1, 6_1, 7_1, 7_2, 8_1.

In some embodiments, the antisense oligonucleotide conjugate comprises CNJ ID NO 1_1. In some embodiments, the antisense oligonucleotide conjugate consists of CNJ ID NO 1_1.

The invention provides an antisense oligonucleotide conjugate selected from the following:

| (SEQ ID NO 1, CMP ID NO 1_1, CNJ ID NO 1_1) | |
|---|---|
| (a) | [5gn2c6]P.[dR](C)P.[dR](A)P.[LR](A)[sP].[LR](G)[sP].[dR](G)[sP].[dR](C)[sP].[LR] |
| | (A)[sP].[dR](T)[sP].[dR](T)[sP].[dR](A)[sP].[dR](T)[sP].[dR](A)[sP].[dR](G)[sP].[dR](G) |
| | [sP].[dR](T)[sP].[dR](T)[sP].[dR](T)[sP].[LR](T)[sP].[LR](T)[sP].[LR]([5meC]), |
| (SEQ ID NO 6, CMP ID NO 6_1, CNJ ID NO 6_1) | |
| (b) | [5gn2c6]P.[dR](C)P.[dR](A)P.[LR](G)[sP].[LR]([5meC])[sP].[dR](A)[sP].[dR](T)[sP]. |
| | [dR](T)[sP].[dR](A)[sP].[dR](T)[sP].[dR](A)[sP].[dR](G)[sP].[dR](G)[sP].[dR](T)[sP]. |
| | [dR](T)[sP].[dR](T)[sP].[LR](T)[sP].[LR](T)[sP].[LR]([5meC])[sP].[LR](T), |
| (SEQ ID NO 7, CMP ID NO 7_1, CNJ ID NO 7_1) | |
| (c) | [5gn2c6]P.[dR](C)P.[dR](A)P.[MOE](G)[P].[LR](G)[sP].[dR](C)[sP].[LR](A)[sP].[dR] |
| | (T)[sP].[dR](T)[sP].[dR](A)[sP].[dR](T)[sP].[R](A)[sP].[dR](G)[sP].[dR](G)[sP].[dR](T) |
| | [sP].[dR](T)[sP].[LR](T)[sP].[MOE](T)[sP].[MOE](T)[sP].[MOE]([5meC]), |
| (SEQ ID NO 7, CMP ID NO 7_2, CNJ ID NO 7_2) | |
| (d) | [5gn2c6]P.[dR](C)P.[dR](A)P.[LR](G)[sP].[LR](G)[sP].[dR](C)[sP].[LR](A)[sP].[dR](T) |
| | [sP].[dR](T)[sP].[dR](A)[sP].[dR](T)[sP].[mR](A)[sP].[dR](G)[sP].[dR](G)[sP].[dR](T) |
| | [sP].[dR](T)[sP].[dR](T)[sP].[LR](T)[sP].[LR](T)[sP].[LR]([5meC]), |
| (SEQ ID NO 8, CMP ID NO 8_1, CNJ ID NO 8_1) | |
| (e) | [5gn2c6]P.[dR](C)P.[dR](A)P.[MOE](G)[sP].[MOE](G)[sP].[MOE]([5meC])[sP].[LR](A) |
| | [PS2].[dR](T)[sP].[dR](T)[sP].[dR](A)[sP].[dR](T)[sP].[mR](A)[sP].[dR](G)[sP].[dR](G) |
| | [sP].[dR](T)[sP].[dR](T)[sP].[LR](T)[PS2].[LR](T)[PS2].[LR](T)[PS2].[MOE]([5meC]), |
| (SEQ ID NO 18, CMP ID NO 18_1, CNJ ID NO 18_1) | |
| (f) | [5gn2c6]P.[dR](C)P.[dR](A)P.[LR](A)[PS2].[LR](G)[sP].[dR](G)[sP].[dR](C)[sP].[LR] |
| | (A)[sP].[dR](T)[sP].[dR](T)[sP].[dR](A)[sP].[dR](T)[sP].[dR](A)[sP].[dR](G)[sP].[dR](G) |
| | [sP].[mR](U)[sP].[dR](T)[sP].[dR](T)[sP].[LR](T)[sP].[LR](T)[PS2].[LR]([5meC]), |
| (SEQ ID NO 19, CMP ID NO 19_1, CNJ ID NO 19_1) | |
| (g) | [5gn2c6]P.[dR](C)P.[dR](A)P.[LR](A)[PS2].[LR](G)[sP].[dR](G)[sP].[dR](C)[sP].[LR] |
| | (A)[sP].[mR](U)[sP].[dR](T)[sP].[dR](A)[sP].[dR](T)[sP].[dR](A)[sP].[dR](G)[sP].[dR] |
| | (G)[sP].[dR](T)[sP].[dR](T)[sP].[dR](T)[sP].[LR](T)[sP].[LR](T)[PS2].[LR]([5meC]), |
| (SEQ ID NO 20, CMP ID NO 20_1, CNJ ID NO 20_1) | |
| (h) | [5gn2c6]P.[dR](C)P.[dR](A)P.[LR](G)[PS2].[LR]([5meC])[sP].[dR](A)[sP].[dR](T)[sP]. |
| | [dR](T)[sP].[dR](A)[sP].[dR](T)[sP].[dR](A)[sP].[dR](G)[sP].[dR](G)[sP].[dR](T)[sP]. |
| | [LR](T)[sP].[dR](T)[sP].[dR](T)[sP].[dR](T)[sP].[dR](C)[sP].[LR](T)[PS2].[LR]([5meC]), |
| (SEQ ID NO 20, CMP ID NO 20_2, CNJ ID NO 20_2) | |
| (i) | [5gn2c6]P.[dR](C)P.[dR](A)P.[LR](G)[PS2].[LR]([5meC])[sP].[mR](A)[sP].[dR](T)[sP]. |
| | [dR](T)[sP].[dR](A)[sP].[dR](T)[sP].[dR](A)[sP].[dR](G)[sP].[dR](G)[sP].[dR](T)[sP]. |
| | [LR](T)[sP].[dR](T)[sP].[dR](T)[sP].[dR](T)[sP].[dR](C)[sP].[LR](T)[PS2].[LR]([5meC]), |
| (SEQ ID NO 20, CMP ID NO 20_3, CNJ ID NO 20_3) | |
| (j) | [5gn2c6]P.[dR](C)P.[dR](A)P.[LR](G)[PS2].[LR]([5meC])[sP].[dR](A)[sP].[dR](T)[sP]. |
| | [dR](T)[sP].[dR](A)[sP].[dR](T)[P].[mR](A)[sP].[dR](G)[sP].[dR](G)[sP].[dR](T)[sP]. |
| | [LR](T)[sP].[dR](T)[sP].[dR](T)[sP].[dR](T)[sP].[dR](C)[sP].[LR](T)[PS2].[LR]([5meC]), |
| (SEQ ID NO 20, CMP ID NO 20_4, CNJ ID NO 20_4) | |
| (k) | [5gn2c6]P.[dR](C)P.[dR](A)P.[LR](G)[PS2].[LR]([5meC])[sP].[dR](A)[sP].[dR](T)[sP]. |
| | [dR](T)[sP].[dR](A)[sP].[dR](T)[sP].[dR](A)[sP].[mR](G)[sP].[dR](G)[sP].[dR](T)[sP]. |
| | [LR](T)[sP].[dR](T)[sP].[dR](T)[sP].[dR](T)[sP].[dR](C)[sP].[LR](T)[PS2].[LR]([5meC]), |
| (SEQ ID NO 20, CMP ID NO 20_5, CNJ ID NO 20_5) | |
| (l) | [5gn2c6]P.[dR](C)P.[dR](A)P.[LR](G)[PS2].[LR]([5meC])[sP].[dR](A)[sP].[dR](T)[sP]. |
| | [dR](T)[sP].[dR](A)[sP].[dR](T)[sP].[dR](A)[sP].[dR](G)[sP].[dR](G)[sP].[dR](T)[sP]. |
| | [LR](T)[sP].[dR](T)[sP].[dR](T)[sP].[dR](T)[sP].[dR](C)[sP].[LR](T)[PS2].[MOE] |
| | ([5meC]), |
| (SEQ ID NO 20, CMP ID NO 20_6, CNJ ID NO 20_6) | |
| (m) | [5gn2c6]P.[dR](C)P.[dR](A)P.[LR](G)[PS2].[LR]([5meC])[sP].[dR](A)[sP].[dR](T)[sP]. |
| | [dR](T)[sP].[dR](A)[sP].[dR](T)[sP].[dR](A)[sP].[dR](G)[sP].[mR](G)[P].[dR](T)[sP]. |
| | [LR](T)[sP].[dR](T)[sP].[dR](T)[sP].[dR](T)[sP].[dR](C)[sP].[LR](T)[PS2].[LR]([5meC]), |
| (SEQ ID NO 6, CMP ID NO 6_2, CNJ ID NO 6_2) | |
| (n) | [5gn2c6]P.[dR](C)P.[dR](A)P.[LR](G)[PS2].[LR]([5meC])[sP].[LR](A)[sP].[dR](T)[sP]. |
| | [dR](T)[sP].[dR](A)[sP].[dR](T)[sP].[dR](A)[sP].[dR](G)[sP].[dR](G)[sP].[dR](T)[sP]. |
| | [dR](T)[sP].[dR](T)[sP].[LR](T)[sP].[dR](T)[sP].[LR]([5meC])[PS2].[LR](T), |
| (SEQ ID NO 6, CMP ID NO 6_3, CNJ ID NO 6_3) | |
| (o) | [5gn2c6]P.[dR](C)P.[dR](A)P.[LR](G)[PS2].[LR]([5meC])[sP].[LR](A)[sP].[dR](T)[sP]. |
| | [dR](T)[sP].[dR](A)[sP].[dR](T)[sP].[mR](A)[sP].[dR](G)[sP].[dR](G)[sP].[dR](T)[sP]. |
| | [dR](T)[sP].[dR](T)[sP].[LR](T)[sP].[dR](T)[sP].[LR]([5meC])[PS2].[LR](T), |
| (SEQ ID NO 6, CMP ID NO 6_4, CNJ ID NO 6_4) | |
| (p) | [5gn2c6]P.[dR](C)P.[dR](A)P.[LR](G)[PS2].[LR]([5meC])[sP].[LR](A)[sP].[dR](T)[sP]. |
| | [dR](T)[sP].[dR](A)[sP].[dR](T)[sP].[dR](A)[sP].[mR](G)[sP].[dR](G)[sP].[dR](T)[sP]. |
| | [dR](T)[sP].[dR](T)[sP].[LR](T)[sP].[dR](T)[sP].[LR]([5meC])[PS2].[LR](T), |
| (SEQ ID NO 6, CMP ID NO 6_5, CNJ ID NO 6_5) | |
| (q) | [5gn2c6]P.[dR](C)P.[dR](A)P.[LR](G)[PS2].[LR]([5meC])[sP].[mR](A)[sP].[dR](T)[sP]. |
| | [dR](T)[sP].[dR](A)[sP].[dR](T)[sP].[dR](A)[sP].[dR](G)[sP].[dR](G)[sP].[dR](T)[sP]. |
| | [dR](T)[sP].[dR](T)[sP].[LR](T)[sP].[LR](T)[sP].[LR]([5meC])[PS2].[LR](T), |
| (SEQ ID NO 6, CMP ID NO 6_6, CNJ ID NO 6_6) | |
| (r) | [5gn2c6]P.[dR](C)P.[dR](A)P.[LR](G)[PS2].[LR]([5meC])[sP].[dR](A)[sP].[dR](T)[sP]. |
| | [dR](T)[sP].[dR](A)[sP].[dR](T)[sP].[mR](A)[sP].[dR](G)[sP].[dR](G)[sP].[dR](T)[sP]. |
| | [dR](T)[sP].[dR](T)[sP].[LR](T)[sP].[LR](T)[sP].[LR]([5meC])[PS2].[LR](T), |
| (SEQ ID NO 6, CMP ID NO 6_7, CNJ ID NO 6_7) | |
| (s) | [5gn2c6]P.[dR](C)P.[dR](A)P.[LR](G)[PS2].[LR]([5meC])[sP].[dR](A)[sP].[dR](T)[sP]. |
| | [dR](T)[sP].[dR](A)[sP].[dR](T)[sP].[dR](A)[sP].[mR](G)[sP].[dR](G)[sP].[dR](T)[sP]. |
| | [dR](T)[sP].[dR](T)[sP].[LR](T)[sP].[LR](T)[sP].[LR]([5meC])[PS2].[LR](T), |
| (SEQ ID NO 6, CMP ID NO 6_8, CNJ ID NO 6_8) | |
| (t) | [5gn2c6]P.[dR](C)P.[dR](A)P.[LR](G)[PS2].[LR]([5meC])[sP].[dR](A)[sP].[dR](T)[sP]. |
| | [dR](T)[sP].[dR](A)[sP].[dR](T)[sP].[dR](A)[sP].[dR](G)[sP].[mR](G)[sP].[dR](T)[sP]. |
| | [dR](T)[sP].[dR](T)[sP].[LR](T)[sP].[LR](T)[sP].[LR]([5meC])[PS2].[LR](T), |
| (SEQ ID NO 6, CMP ID NO 6_9, CNJ ID NO 6_9) | |
| (u) | [5gn2c6]P.[dR](C)P.[dR](A)P.[MOE](G)[sP].[LR]([5meC])[PS2].[dR](A)[sP].[dR](T) |
| | [sP].[dR](T)[sP].[dR](A)[sP].[dR](T)[sP].[dR](A)[sP].[dR](G)[sP].[dR](G)[sP].[dR](T) |
| | [sP].[dR](T)[sP].[dR](T)[sP].[LR](T)[sP].[LR](T)[sP].[LR]([5meC])[PS2].[MOE](T), |
| (SEQ ID NO 6, CMP ID NO 6_10, CNJ ID NO 6_10) | |
| (v) | [5gn2c6]P.[dR](C)P.[dR](A)P.[MOE](G)[sP].[LR]([5meC])[PS2].[dR](A)[sP].[dR](T) |
| | [sP].[dR](T)[sP].[dR](A)[sP].[dR](T)[sP].[dR](A)[sP].[dR](G)[sP].[dR](G)[sP].[dR](T) |
| | [sP].[dR](T)[sP].[dR](T)[sP].[LR](T)[sP].[LR](T)[PS2].[MOE]([5meC])[sP].[MOE](T), |
| (SEQ ID NO 6, CMP ID NO 6_11, CNJ ID NO 6_11) | |
| (w) | [5gn2c6]P.[dR](C)P.[dR](A)P.[MOE](G)[sP].[LR]([5meC])[PS2].[dR](A)[sP].[dR](T) |
| | [sP].[dR](T)[sP].[dR](A)[sP].[dR](T)[sP].[dR](A)[sP].[dR](G)[sP].[dR](G)[sP].[dR](T) |
| | [sP].[dR](T)[sP].[dR](T)[sP].[LR](T)[sP].[LR](T)[PS2].[LR]([5meC])[PS2].[MOE](T), |
| (SEQ ID NO 6, CMP ID NO 6_12, CNJ ID NO 6_12) | |
| (x) | [5gn2c6]P.[dR](C)P.[dR](A)P.[MOE](G)[sP].[LR]([5meC])[PS2].[dR](A)[sP].[dR](T) |
| | [sP].[dR](T)[sP].[dR](A)[sP].[dR](T)[sP].[dR](A)[sP].[dR](G)[sP].[dR](G)[sP].[dR](T) |
| | [sP].[dR](T)[sP].[dR](T)[sP].[LR](T)[PS2].[LR](T)[PS2].[MOE]([5meC])[sP].[MOE](T), |
| (SEQ ID NO 21, CMP ID NO 21_1, CNJ ID NO 21_1) | |
| (y) | [5gn2c6]P.[dR](C)P.[dR](A)P.[LR](G)[PS2].[LR]([5meC])[sP].[dR](A)[sP].[dR](T)[sP]. |
| | [dR](T)[sP].[dR](A)[sP].[dR](T)[sP].[dR](A)[sP].[dR](G)[sP].[dR](G)[sP].[dR](T)[sP]. |
| | [LR](T)[sP].[dR](T)[sP].[dR](T)[sP].[mR](U)[sP].[dR](C)[sP].[LR](T)[PS2].[LR]([5meC]), |
| (SEQ ID NO 22, CMP ID NO 22_1, CNJ ID NO 22_1) | |
| (z) | [5gn2c6]P.[dR](C)P.[dR](A)P.[LR](G)[PS2].[LR]([5meC])[sP].[dR](A)[sP].[dR](T)[sP]. |
| | [dR](T)[sP].[dR](A)[sP].[dR](T)[sP].[dR](A)[sP].[dR](G)[sP].[dR](G)[sP].[dR](T)[sP]. |
| | [LR](T)[sP].[dR](T)[sP].[mR](U)[sP].[dR](T)[sP].[dR](C)[sP].[LR](T)[PS2].[LR]([5meC]), |
| (SEQ ID NO 23, CMP ID NO 23_1, CNJ ID NO 23_1) | |
| (aa) | [5gn2c6]P.[dR](C)P.[dR](A)P.[LR](G)[PS2].[LR]([5meC])[sP].[dR](A)[sP].[dR](T)[sP]. |
| | [dR](T)[sP].[dR](A)[sP].[dR](T)[P].[dR](A)[sP].[dR](G)[sP].[dR](G)[sP].[dR](T)[sP]. |
| | [LR](T)[sP].[mR](U)[sP].[dR](T)[sP].[dR](T)[sP].[dR](C)[sP].[LR](T)[PS2].[LR]([5meC]), |
| (SEQ ID NO 24, CMP ID NO 24_1, CNJ ID NO 24_1) | |
| (ab) | [5gn2c6]P.[dR](C)P.[dR](A)P.[LR](G)[PS2].[LR]([5meC])[sP].[dR](A)[sP].[dR](T)[sP]. |
| | [dR](T)[sP].[dR](A)[sP].[dR](T)[sP].[dR](A)[sP].[dR](G)[sP].[dR](G)[sP].[dR](T)[sP]. |
| | [dR](T)[sP].[mR](U)[sP].[LR](T)[sP].[LR](T)[sP].[LR]([5meC])[PS2].[LR](T), |
| (SEQ ID NO 25, CMP ID NO 25_1, CNJ ID NO 25_1) | |
| (ac) | [5gn2c6]P.[dR](C)P.[dR](A)P.[LR](G)[PS2].[LR]([5meC])[sP].[LR](A)[sP].[dR](T)[sP]. |
| | [dR](T)[sP].[dR](A)[sP].[dR](T)[sP].[dR](A)[sP].[dR](G)[sP].[dR](G)[sP].[dR](T)[sP]. |
| | [mR](U)[sP].[dR](T)[sP].[LR](T)[sP].[dR](T)[sP].[LR]([5meC])[PS2].[LR](T), |
| (SEQ ID NO 25, CMP ID NO 25_2, CNJ ID NO 25_2) | |
| (ad) | [5gn2c6]P.[dR](C)P.[dR](A)P.[LR](G)[PS2].[LR]([5meC])[sP].[dR](A)[sP].[dR](T)[sP]. |
| | [dR](T)[sP].[dR](A)[sP].[dR](T)[sP].[dR](A)[sP].[dR](G)[sP].[dR](G)[sP].[dR](T)[sP]. |
| | [mR](U)[sP].[dR](T)[sP].[LR](T)[sP].[LR](T)[sP].[LR]([5meC])[PS2].[LR](T), |
| (SEQ ID NO 26, CMP ID NO 26_1, CNJ ID NO 26_1) | |
| (ae) | [5gn2c6]P.[dR](C)P.[dR](A)P.[LR](G)[PS2].[LR]([5meC])[sP].[dR](A)[sP].[dR](T)[sP]. |
| | [dR](T)[sP].[dR](A)[sP].[dR](T)[sP].[dR](A)[sP].[dR](G)[sP].[dR](G)[sP].[mR](U)[sP]. |
| | [LR](T)[sP].[dR](T)[sP].[dR](T)[sP].[dR](T)[sP].[dR](C)[sP].[LR](T)[PS2].[LR]([5meC]), |
| (SEQ ID NO 27, CMP ID NO 27_1, CNJ ID NO 27_1) | |
| (af) | [5gn2c6]P.[dR](C)P.[dR](A)P.[LR](G)[PS2].[LR]([5meC])[sP].[LR](A)[sP].[dR](T)[sP]. |
| | [dR](T)[sP].[dR](A)[sP].[dR](T)[sP].[dR](A)[sP].[dR](G)[sP].[dR](G)[sP].[mR](U)[sP]. |
| | [dR](T)[sP].[dR](T)[sP].[LR](T)[sP].[dR](T)[sP].[LR]([5meC])[PS2].[LR](T), |
| (SEQ ID NO 27, CMP ID NO 27_2, CNJ ID NO 27_2) | |
| (ag) | [5gn2c6]P.[dR](C)P.[dR](A)P.[LR](G)[PS2].[LR]([5meC])[sP].[dR](A)[sP].[dR](T)[sP]. |
| | [dR](T)[sP].[dR](A)[sP].[dR](T)[sP].[dR](A)[sP].[dR](G)[sP].[dR](G)[sP].[mR](U)[sP]. |
| | [dR](T)[sP].[dR](T)[sP].[LR](T)[sP].[LR](T)[sP].[LR]([5meC])[PS2].[LR](T), |
| (SEQ ID NO 28, CMP ID NO 28_1, CNJ ID NO 28_1) | |
| (ah) | [5gn2c6]P.[dR](C)P.[dR](A)P.[LR](G)[PS2].[LR]([5meC])[sP].[dR](A)[sP].[dR](T)[sP]. |
| | [dR](T)[sP].[dR](A)[sP].[mR](U)[sP].[dR](A)[sP].[dR](G)[sP].[dR](G)[sP].[dR](T)[sP]. |
| | [dR](T)[sP].[dR](T)[sP].[LR](T)[sP].[LR](T)[sP].[LR]([5meC])[PS2].[LR](T), |
| (SEQ ID NO 29, CMP ID NO 29_1, CNJ ID NO 29_1) | |
| (ai) | [5gn2c6]P.[dR](C)P.[dR](A)P.[LR](G)[PS2].[LR]([5meC])[sP].[dR](A)[sP].[dR](T)[sP]. |
| | [mR](U)[sP].[dR](A)[sP].[dR](T)[sP].[dR](A)[sP].[dR](G)[sP].[dR](G)[sP].[dR](T)[sP]. |
| | [dR](T)[sP].[dR](T)[sP].[LR](T)[sP].[LR](T)[sP].[LR]([5meC])[PS2].[LR](T), |
| and | |
| (SEQ ID NO 30, CMP ID NO 30_1, CNJ ID NO 30_1) | |
| (aj) | [5gn2c6]P.[dR](C)P.[dR](A)P.[LR](G)[PS2].[LR]([5meC])[sP].[dR](A)[sP].[mR](U)[sP]. |
| | [dR](T)[sP].[dR](A)[sP].[dR](T)[sP].[dR](A)[sP].[dR](G)[sP].[dR](G)[sP].[dR](T)[sP]. |
| | [dR](T)[sP].[dR](T)[sP].[LR](T)[sP].[LR](T)[sP].[LR]([5meC])[PS2].[LR](T), |

wherein
(A) is a nucleoside comprising an adenine nucleobase,
(C) is a nucleoside comprising a cytosine nucleobase,
([5meC]) is a nucleoside comprising a 5-methyl cytosine nucleobase,
([PPG]) is a nucleoside comprising a 7-deaza-8-aza guanine nucleobase,
(G) is a nucleoside comprising a guanine nucleobase,
(U) is a nucleoside comprising a uracil nucleobase,
(T) is a nucleoside comprising a thymine nucleobase,
[LR] indicates that the following nucleoside is a beta-D-oxy-LNA nucleoside,
[dR] indicates that the following nucleoside is a DNA nucleoside,
[mR] indicates that the following nucleoside is a 2′-O-methyl RNA nucleoside,
[MOE] indicates that the following nucleoside is a 2′-O-methoxyethyl RNA (MOE RNA) nucleoside,
[sP] indicates a phosphorothioate internucleoside linkage between the preceding and following nucleoside,
[PS2] indicates a phosphorodithioate internucleoside linkage between the preceding and following nucleoside,
P indicates a phosphodiester internucleoside linkage between the preceding nucleoside or moiety and the following nucleoside, and
[5gn2c6] is an aminohexyl conjugated tri(N-acetyl-galactosamine) moiety as shown in FIG. 8.

In some embodiments, the antisense oligonucleotide conjugate is selected from the following:

| (SEQ ID NO 1, CMP ID NO 1_1, CNJ ID NO 1_1) | |
|---|---|
| (a) | [5gn2c6]P.[dR](C)P.[dR](A)P.[LR](A)[sP].[LR](G)[sP].[dR](G)[sP].[dR](C)[sP].[LR] |
| | (A)[sP].[dR](T)[sP].[dR](T)[sP].[dR](A)[sP].[dR](T)[sP].[dR](A)[sP].[dR](G)[sP].[dR](G) |
| | [sP].[dR](T)[sP].[dR](T)[sP].[dR](T)[sP].[LR](T)[sP].[LR](T)[sP].[LR]([5meC]), |
| (SEQ ID NO 6, CMP ID NO 6_1, CNJ ID NO 6_1) | |
| (b) | [5gn2c6]P.[dR](C)P.[dR](A)P.[LR](G)[sP].[LR]([5meC])[sP].[dR](A)[sP].[dR](T)[sP]. |
| | [dR](T)[sP].[dR](A)[sP].[dR](T)[sP].[dR](A)[sP].[dR](G)[sP].[dR](G)[sP].[dR](T)[sP]. |
| | [dR](T)[sP].[dR](T)[sP].[LR](T)[sP].[LR](T)[sP].[LR]([5meC])[sP].[LR](T), |
| (SEQ ID NO 7, CMP ID NO 7_1, CNJ ID NO 7_1) | |
| (c) | [5gn2c6]P.[dR](C)P.[dR](A)P.[MOE](G)[sP].[LR](G)[sP].[dR](C)[sP].[LR](A)[sP].[dR] |
| | (T)[sP].[dR](T)[sP].[dR](A)[sP].[dR](T)[sP].[dR](A)[sP].[dR](G)[sP].[dR](G)[sP].[dR](T) |
| | [sP].[dR](T)[sP].[LR](T)[sP].[MOE](T)[sP].[MOE](T)[sP].[MOE]([5meC]), |
| (SEQ ID NO 7, CMP ID NO 7_2, CNJ ID NO 7_2) | |
| (d) | [5gn2c6]P.[dR](C)P.[dR](A)P.[LR](G)[sP].[LR](G)[sP].[dR](C)[sP].[LR](A)[sP].[dR](T) |
| | [sP].[dR](T)[sP].[dR](A)[sP].[dR](T)[sP].[mR](A)[sP].[dR](G)[sP].[dR](G)[sP].[dR](T) |
| | [sP].[dR](T)[sP].[dR](T)[sP].[LR](T)[sP].[LR](T)[sP].[LR]([5meC]), |
| and | |
| (SEQ ID NO 8, CMP ID NO 8_1, CNJ ID NO 8_1) | |
| (e) | [5gn2c6]P.[dR](C)P.[dR](A)P.[MOE](G)[sP].[MOE](G)[sP].[MOE]([5meC])[sP].[LR](A) |
| | [PS2].[dR](T)[sP].[dR](T)[sP].[dR](A)[sP].[dR](T)[sP].[mR](A)[sP].[dR](G)[sP].[dR](G) |
| | [sP].[dR](T)[sP].[dR](T)[sP].[LR](T)[PS2].[LR](T)[PS2].[LR](T)[PS2].[MOE]([5meC]). |

In some embodiments, the antisense oligonucleotide conjugate comprises the following:

(SEQ ID NO 1, CMP ID NO 1_1, CNJ ID NO 1_1)
[5gn2c6]P.[dR](C)P.[dR](A)P.[LR](A)[sP].[LR](G)[sP].[dR](G)[sP].[dR](C)[sP].[LR](A)[sP].[dR]
(T)[sP].[dR](T)[sP].[dR](A)[sP].[dR](T)[sP].[dR](A)[sP].[dR](G)[sP].[dR](G)[sP].[dR](T)[sP].
[dR](T)[sP].[dR](T)[sP].[LR](T)[sP].[LR](T)[sP].[LR]([5meC]).

In some embodiments, the antisense oligonucleotide conjugate consists of the following:

(SEQ ID NO 1, CMP ID NO 1_1, CNJ ID NO 1_1)
[5gn2c6]P.[dR](C)P.[dR](A)P.[LR](A)[sP].[LR](G)[P].[dR](G)[sP].[dR](C)[sP].[LR](A)[sP].[dR]
(T)[sP].[dR](T)[sP].[dR](A)[sP].[dR](T)[sP].[dR](A)[sP].[dR](G)[sP].[dR](G)[sP].[dR](T)[sP].
[dR](T)[sP].[dR](T)[sP].[LR](T)[sP].[LR](T)[sP].[LR]([5meC]).

The invention provides an antisense oligonucleotide conjugate which is CNJ ID NO 1_1 as depicted in FIG. 1.

Targeting A1CF 16970-16989

The antisense oligonucleotide or antisense oligonucleotide conjugate of the invention may bind a target sequence within the region defined by positions 16970-16989 of the A1CF pre-mRNA (SEQ ID NO 45).

Thus, the invention provides an antisense oligonucleotide of 12 to 30 nucleotides in length, which comprises a contiguous nucleotide sequence of 12 to 30 nucleotides in length, wherein the contiguous nucleotide sequence is capable of binding to a target sequence in an A1CF mRNA, wherein the target sequence is a sequence of at least 17 contiguous nucleotides within AAGUAAAAUUAACAUGUCCA (SEQ ID NO 43) (positions 16970-16989 of SEQ ID NO 45), and wherein the antisense oligonucleotide is capable of reducing A1CF expression.

The invention also provides an antisense oligonucleotide conjugate comprising an antisense oligonucleotide covalently attached to at least one conjugate moiety, wherein the antisense oligonucleotide is an antisense oligonucleotide of 12 to 30 nucleotides in length, which comprises a contiguous nucleotide sequence of 12 to 30 nucleotides in length, wherein the contiguous nucleotide sequence is capable of binding to a target sequence in an A1CF mRNA, wherein the target sequence is a sequence of at least 17 contiguous nucleotides within AAGUAAAAUUAACAUGUCCA (SEQ ID NO 43) (positions 16970-16989 of SEQ ID NO 45), and wherein the antisense oligonucleotide is capable of reducing A1CF expression.

In some embodiments of the antisense oligonucleotide or the antisense oligonucleotide conjugate of the invention, the target sequence is a sequence of 17, 18, 19 or 20 contiguous nucleotides within SEQ ID NO 43.

In some embodiments, the target sequence is

(SEQ ID NO 33)
AAGUAAAAUUAACAUGUCC
(positions 16970-16988 of SEQ ID NO 45)
or
(SEQ ID NO 38)
GUAAAAUUAACAUGUCCA
(positions 16972-16989 of SEQ ID NO 45).

In some embodiments, the target sequence is SEQ ID NO 33.

In some embodiments, the sequence of the antisense oligonucleotide and/or the sequence of the contiguous nucleotide sequence comprises or consists of a sequence having at least 80% identity to any one of the following sequences:

(SEQ ID NO 3)
GGACATGTTAATTTTACTT,
(SEQ ID NO 9)
TGGACAUGTTAATTTTAE,
and
(SEQ ID NO 10)
GGACATGUTAATTTTACTT.

In some embodiments, the sequence of the antisense oligonucleotide and/or the sequence of the contiguous nucleotide sequence comprises or consists of a sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to any one of SEQ ID NOs 3, 9 or 10.

In some embodiments, the sequence of the antisense oligonucleotide and/or the sequence of the contiguous nucleotide sequence comprises or consists of a sequence having at least 80% identity to SEQ ID NO 3.

In some embodiments, the sequence of the antisense oligonucleotide and/or the sequence of the contiguous nucleotide sequence comprises or consists of a sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO 3.

In some embodiments, the sequence of the antisense oligonucleotide and/or the sequence of the contiguous nucleotide sequence consists of SEQ ID NO 3. In some embodiments, the sequence of the contiguous nucleotide sequence consists of SEQ ID NO 3. In some embodiments, the sequence of the antisense oligonucleotide consists of SEQ ID NO 3.

In some embodiments, the antisense oligonucleotide comprises or consists of any one of CMP ID NOs 3_1, 9_1, and 10_1.

In some embodiments, the antisense oligonucleotide comprises CMP ID NO 3_1. In some embodiments, the antisense oligonucleotide consists of CMP ID NO 3_1.

The invention provides an antisense oligonucleotide comprising or consisting of a compound selected from the following:

(a)
(SEQ ID NO 3, CMP ID NO 3_1)
[LR](G)[sP].[LR](G)[sP].[mR](A)[sP].[dR](C)[sP].[dR](A)[sP].[dR](T)[sP].[LR](G)[sP].
[dR](T)[sP].[dR](T)[sP].[dR](A)[sP].[dR](A)[sP].[dR](T)[sP].[dR](T)[sP].[dR](T)[sP].[dR]
(T)[sP].[dR](A)[sP].[dR](C)[sP].[LR](T)[PS2].[LR](T),
(b)
(SEQ ID NO 9, CMP ID NO 9_1)
[LR](T)[PS2].[LR](G)[sP].[dR](G)[sP].[LR](A)[sP].[dR](C)[sP].[dR](A)[sP].[mR](U)[sP].
[dR](G)[sP].[dR](T)[sP].[LR](T)[sP].[dR](A)[sP].[dR](A)[sP].[dR](T)[sP].[dR](T)[sP].
[dR](T)[sP].[dR](T)[sP].[LR](A)[PS2].[LR]([5meC]),
and
(c)
(SEQ ID NO 10, CMP ID NO 10_1)
[LR](G)[PS2].[LR](G)[PS2].[dR](A)[sP].[dR](C)[sP].[dR](A)[sP].[dR](T)[sP].[LR](G)[sP]
.[mR](U)[sP].[dR](T)[sP].[mR](A)[sP].[dR](A)[sP].[dR](T)[sP].[dR](T)[sP].[dR](T)[sP].
[dR](T)[sP].[dR](A)[sP].[dR](C)[sP].[LR](T)[PS2].[LR](T),

wherein
(A) is a nucleoside comprising an adenine nucleobase,
(C) is a nucleoside comprising a cytosine nucleobase,
([5meC]) is a nucleoside comprising a 5-methyl cytosine nucleobase,
([PPG]) is a nucleoside comprising a 7-deaza-8-aza guanine nucleobase,
(G) is a nucleoside comprising a guanine nucleobase,
(U) is a nucleoside comprising a uracil nucleobase,
(T) is a nucleoside comprising a thymine nucleobase,
[LR] indicates that the following nucleoside is a beta-D-oxy-LNA nucleoside,
[dR] indicates that the following nucleoside is a DNA nucleoside,
[mR] indicates that the following nucleoside is a 2′-O-methyl RNA nucleoside,
[MOE] indicates that the following nucleoside is a 2′-O-methoxyethyl RNA (MOE RNA) nucleoside,
[sP] indicates a phosphorothioate internucleoside linkage between the preceding and following nucleoside, and
[PS2] indicates a phosphorodithioate internucleoside linkage between the preceding and following nucleoside.

In some embodiments, the antisense oligonucleotide comprises the following compound:

(SEQ ID NO 3, CMP ID NO 3_1)
[LR](G)[sP].[LR](G)[sP].[mR](A)[sP].[dR](C)[sP].[dR](A)[sP].[dR](T)[sP].[LR](G)[sP].[dR](T)
[sP].[dR](T)[sP].[dR](A)[sP].[dR](A)[sP].[dR](T)[sP].[dR](T)[sP].[dR](T)[sP].[dR](T)[sP].[dR]
(A)[sP].[dR](C)[sP].[LR](T)[PS2].[LR](T).

In some embodiments, the antisense oligonucleotide consists of the following compound:

(SEQ ID NO 3, CMP ID NO 3_1)
[LR](G)[sP].[LR](G)[sP].[mR](A)[sP].[dR](C)[sP].[dR](A)[sP].[dR](T)[sP].[LR](G)[sP].[dR](T)
[sP].[dR](T)[sP].[dR](A)[sP].[dR](A)[sP].[dR](T)[sP].[dR](T)[sP].[dR](T)[sP].[dR](T)[sP].[dR]
(A)[sP].[dR](C)[sP].[LR](T)[PS2].[LR](T).

In some embodiments, the antisense oligonucleotide conjugate comprises or consists of any one of CNJ ID NOs 3_1, 9_1, or 10_1.

In some embodiments, the antisense oligonucleotide conjugate comprises CNJ ID NO 3_1. In some embodiments, the antisense oligonucleotide conjugate consists of CNJ ID NO 3_1.

The invention provides an antisense oligonucleotide conjugate selected from the following:

(a)
(SEQ ID NO 3, CMP ID NO 3_1, CNJ ID NO 3_1)
[5gn2c6]P.[dR](C)P.[dR](A)P.[LR](G)[sP].[LR](G)[sP].[mR](A)[sP].[dR](C)[sP].[dR]
(A)[sP].[dR](T)[sP].[LR](G)[sP].[dR](T)[sP].[dR](T)[sP].[dR](A)[sP].[dR](A)[sP].[dR](T)
[sP].[dR](T)[sP].[dR](T)[sP].[dR](T)[sP].[dR](A)[sP].[dR](C)[sP].[LR](T)[PS2].[LR](T),
(b)
(SEQ ID NO 9, CMP ID NO 9_1, CNJ ID NO 9_1)
[5gn2c6]P.[dR](C)P.[dR](A)P.[LR](T)[PS2].[LR](G)[sP].[dR](G)[sP].[LR](A)[sP].[dR]
(C)[sP].[dR](A)[sP].[mR](U)[sP].[dR](G)[sP].[dR](T)[sP].[LR](T)[sP].[dR](A)[sP].[dR]
(A)[sP].[dR](T)[sP].[dR](T)[sP].[dR](T)[sP].[dR](T)[P].[LR](A)[PS2].[LR]([5meC]),
and
(c)
(SEQ ID NO 10, CMP ID NO 10_1, CNJ ID NO 10_1)
[5gn2c6]P.[dR](C)P.[dR](A)P.[LR](G)[PS2].[LR](G)[PS2].[dR](A)[sP].[dR](C)[sP].[dR]
(A)[sP].[dR](T)[sP].[LR](G)[sP].[mR](U)[sP].[dR](T)[sP].[mR](A)[P].[dR](A)[sP].[dR]
(T)[sP].[dR](T)[sP].[dR](T)[sP].[dR](T)[sP].[dR](A)[sP].[dR](C)[sP].[LR](T)[PS2].[LR]
(T),

wherein
(A) is a nucleoside comprising an adenine nucleobase,
(C) is a nucleoside comprising a cytosine nucleobase,
([5meC]) is a nucleoside comprising a 5-methyl cytosine nucleobase,
([PPG]) is a nucleoside comprising a 7-deaza-8-aza guanine nucleobase,
(G) is a nucleoside comprising a guanine nucleobase,
(U) is a nucleoside comprising a uracil nucleobase,
(T) is a nucleoside comprising a thymine nucleobase,
[LR] indicates that the following nucleoside is a beta-D-oxy-LNA nucleoside,
[dR] indicates that the following nucleoside is a DNA nucleoside,
[mR] indicates that the following nucleoside is a 2′-O-methyl RNA nucleoside,
[MOE] indicates that the following nucleoside is a 2′-O-methoxyethyl RNA (MOE RNA) nucleoside,
[sP] indicates a phosphorothioate internucleoside linkage between the preceding and following nucleoside,
[PS2] indicates a phosphorodithioate internucleoside linkage between the preceding and following nucleoside,
P indicates a phosphodiester internucleoside linkage between the preceding nucleoside or moiety and the following nucleoside, and
[5gn2c6] is an aminohexyl conjugated tri(N-acetyl-galactosamine) moiety as shown in FIG. 8.

In some embodiments the antisense oligonucleotide conjugate comprises the following:

(SEQ ID NO 3, CMP ID NO 3_1, CNJ ID NO 3_1)
[5gn2c6]P.[dR](C)P.[dR](A)P.[LR](G)[sP].[LR](G)[sP].[mR](A)[sP].[dR](C)[sP].[dR](A)[sP].
[dR](T)[P].[LR](G)[sP].[dR](T)[sP].[dR](T)[sP].[dR](A)[sP].[dR](A)[sP].[dR](T)[sP].[dR](T)
[sP].[dR](T)[sP].[dR](T)[sP].[dR](A)[sP].[dR](C)[sP].[LR](T)[PS2].[LR](T).

In some embodiments the antisense oligonucleotide conjugate consists of the following:

(SEQ ID NO 3, CMP ID NO 3_1, CNJ ID NO 3_1)
[5gn2c6]P.[dR](C)P.[dR](A)P.[LR](G)[sP].[LR](G)[sP].[mR](A)[sP].[dR](C)[sP].[dR](A)[sP].
[dR](T)[sP].[LR](G)[sP].[dR](T)[sP].[dR](T)[sP].[dR](A)[sP].[dR](A)[sP].[dR](T)[sP].[dR](T)
[sP].[dR](T)[sP].[dR](T)[sP].[dR](A)[sP].[dR](C)[sP].[LR](T)[PS2].[LR](T).

The invention provides an antisense oligonucleotide conjugate which is CNJ ID NO 3_1 as depicted in FIG. 3.

Targeting A1CF 26358-26377

The antisense oligonucleotide or antisense oligonucleotide conjugate of the invention may bind a target sequence within the region defined by positions 26358-26377 of the A1CF pre-mRNA (SEQ ID NO 45).

Thus, the invention provides an antisense oligonucleotide of 12 to 30 nucleotides in length, which comprises a contiguous nucleotide sequence of 12 to 30 nucleotides in length, wherein the contiguous nucleotide sequence is capable of binding to a target sequence in an A1CF mRNA, wherein the target sequence is a sequence of at least 17 contiguous nucleotides within AAACACCACAAUCUUAAAAC (SEQ ID NO 39) (positions 26358-26377 of SEQ ID NO 45), and wherein the antisense oligonucleotide is capable of reducing A1CF expression.

The invention also provides an antisense oligonucleotide conjugate comprising an antisense oligonucleotide covalently attached to at least one conjugate moiety, wherein the antisense oligonucleotide is an antisense oligonucleotide of 12 to 30 nucleotides in length, which comprises a contiguous nucleotide sequence of 12 to 30 nucleotides in length, wherein the contiguous nucleotide sequence is capable of binding to a target sequence in an A1CF mRNA, wherein the target sequence is a sequence of at least 17 contiguous nucleotides within AAACACCACAAUCUUAAAAC (SEQ ID NO 39) (positions 26358-26377 of SEQ ID NO 45), and wherein the antisense oligonucleotide is capable of reducing A1CF expression.

In some embodiments of the antisense oligonucleotide or the antisense oligonucleotide conjugate of the invention, the target sequence is a sequence of 17, 18, 19 or 20 contiguous nucleotides within SEQ ID NO 39.

In some embodiments, the target sequence is AAACACCACAAUCUUAAAAC (SEQ ID NO 40) (positions 26358-26377 of SEQ ID NO 45).

In some embodiments, the sequence of the antisense oligonucleotide and/or the sequence of the contiguous nucleotide sequence comprises or consists of a sequence having at least 80% identity to GTTTTAAGATTGTGGTGTTT (SEQ ID NO 11).

In some embodiments, the sequence of the antisense oligonucleotide and/or the sequence of the contiguous nucleotide sequence comprises or consists of a sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% to SEQ ID NO 11.

In some embodiments, the sequence of the antisense oligonucleotide and/or the sequence of the contiguous nucleotide sequence consists of SEQ ID NO 11. In some embodiments, the sequence of the contiguous nucleotide sequence consists of SEQ ID NO 11. In some embodiments, the sequence of the antisense oligonucleotide consists of SEQ ID NO 11.

In some embodiments, the antisense oligonucleotide comprises or consists of any one of CMP ID NOs 11_1, 11_2 or 11_3.

In some embodiments, the antisense oligonucleotide comprises CMP ID NO 11_1. In some embodiments, the antisense oligonucleotide consists of CMP ID NO 11_1.

The invention provides an antisense oligonucleotide comprising or consisting of a compound selected from the following:

(a)
(SEQ ID NO 11, CMP ID NO 11_1)
[LR](G)[sP].[LR](T)[sP].[LR](T)[sP].[dR](T)[sP].[dR](T)[sP].[mR](A)[sP].[dR](A)[sP].
[dR](G)[sP].[dR](A)[sP].[dR](T)[sP].[dR](T)[sP].[dR](G)[sP].[dR](T)[sP].[dR](G)[sP].
[dR](G)[sP].[dR](T)[sP].[dR](G)[sP].[LR](T)[sP].[LR](T)[PS2].[LR](T),
(b)
(SEQ ID NO 11, CMP ID NO 11_2)
[LR](G)[sP].[LR](T)[sP].[LR](T)[sP].[dR](T)[sP].[dR](T)[sP].[dR](A)[sP].[dR](A)[sP].
[mR](G)[sP].[dR](A)[sP].[dR](T)[sP].[dR](T)[sP].[dR](G)[sP].[dR](T)[sP].[dR](G)[sP].
[dR](G)[sP].[dR](T)[sP].[dR](G)[sP].[LR](T)[sP].[LR](T)[sP].[LR](T),
and
(c)
(SEQ ID NO 11, CMP ID NO 11_3)
[LR](G)[sP].[LR](T)[sP].[LR](T)[sP].[dR](T)[sP].[dR](T)[sP].[dR](A)[sP].[dR](A)[sP].
[mR](G)[sP].[dR](A)[sP].[dR](T)[sP].[dR](T)[sP].[dR](G)[sP].[dR](T)[sP].[dR](G)[sP].
[dR](G)[sP].[dR](T)[sP].[dR](G)[sP].[LR](T)[sP].[LR](T)[PS2].[LR](T),

wherein
(A) is a nucleoside comprising an adenine nucleobase,
(C) is a nucleoside comprising a cytosine nucleobase,
([5meC]) is a nucleoside comprising a 5-methyl cytosine nucleobase,
([PPG]) is a nucleoside comprising a 7-deaza-8-aza guanine nucleobase,
(G) is a nucleoside comprising a guanine nucleobase,
(U) is a nucleoside comprising a uracil nucleobase,
(T) is a nucleoside comprising a thymine nucleobase,
[LR] indicates that the following nucleoside is a beta-D-oxy-LNA nucleoside,
[dR] indicates that the following nucleoside is a DNA nucleoside,
[mR] indicates that the following nucleoside is a 2′-O-methyl RNA nucleoside,
[MOE] indicates that the following nucleoside is a 2′-O-methoxyethyl RNA (MOE RNA) nucleoside,
[sP] indicates a phosphorothioate internucleoside linkage between the preceding and following nucleoside, and
[PS2] indicates a phosphorodithioate internucleoside linkage between the preceding and following nucleoside.

In some embodiments, the antisense oligonucleotide comprises the following compound:

(SEQ ID NO 11, CMP ID NO 11_1)
[LR](G)[P].[LR](T)[sP].[LR](T)[sP].[dR](T)[sP].[dR](T)[sP].[mR](A)[sP].[dR](A)[sP].[dR](G)
[sP].[dR](A)[sP].[dR](T)[sP].[dR](T)[sP].[dR](G)[sP].[dR](T)[sP].[dR](G)[sP].[dR](G)[sP].[dR]
(T)[sP].[dR](G)[sP].[LR](T)[sP].[LR](T)[PS2].[LR](T).

In some embodiments, the antisense oligonucleotide consists of the following compound:

(SEQ ID NO 11, CMP ID NO 11_1)
[LR](G)[sP].[LR](T)[sP].[LR](T)[sP].[dR](T)[sP].[dR](T)[sP].[mR](A)[sP].[dR](A)[sP].[dR](G)
[sP].[dR](A)[sP].[dR](T)[sP].[dR](T)[sP].[dR](G)[sP].[dR](T)[sP].[dR](G)[sP].[dR](G)[sP].[dR]
(T)[sP].[dR](G)[sP].[LR](T)[sP].[LR](T)[PS2].[LR](T).

In some embodiments, the antisense oligonucleotide conjugate comprises or consists of any one of CNJ ID NOs 1_1, 11_2 or 11_3.

In some embodiments, the antisense oligonucleotide conjugate comprises CNJ ID NO 11_1.

In some embodiments, the antisense oligonucleotide conjugate consists of CNJ ID NO 11_1.

The invention provides an antisense oligonucleotide conjugate selected from the following:

(a)
(SEQ ID NO 11, CMP ID NO 11_1)
[LR](G)[sP].[LR](T)[sP].[LR](T)[sP].[dR](T)[sP].[dR](T)[sP].[mR](A)[sP].[dR](A)[sP].
[dR](G)[sP].[dR](A)[sP].[dR](T)[sP].[dR](T)[sP].[dR](G)[sP].[dR](T)[sP].[dR](G)[sP].
[dR](G)[sP].[dR](T)[sP].[dR](G)[sP].[LR](T)[sP].[LR](T)[PS2].[LR](T),
(b)
(SEQ ID NO 11, CMP ID NO 11_2)
[LR](G)[sP].[LR](T)[sP].[LR](T)[sP].[dR](T)[sP].[dR](T)[sP].[dR](A)[sP].[dR](A)[sP].
[mR](G)[sP].[dR](A)[sP].[dR](T)[sP].[dR](T)[sP].[dR](G)[sP].[dR](T)[sP].[dR](G)[sP].
[dR](G)[sP].[dR](T)[sP].[dR](G)[sP].[LR](T)[sP].[LR](T)[sP].[LR](T),
and
(c)
(SEQ ID NO 11, CMP ID NO 11_3)
[LR](G)[sP].[LR](T)[sP].[LR](T)[sP].[dR](T)[sP].[dR](T)[sP].[dR](A)[sP].[dR](A)[sP].
[mR](G)[sP].[dR](A)[sP].[dR](T)[sP].[dR](T)[sP].[dR](G)[sP].[dR](T)[sP].[dR](G)[sP].
[dR](G)[sP].[dR](T)[sP].[dR](G)[sP].[LR](T)[sP].[LR](T)[PS2].[LR](T),

wherein
(A) is a nucleoside comprising an adenine nucleobase,
(C) is a nucleoside comprising a cytosine nucleobase,
([5meC]) is a nucleoside comprising a 5-methyl cytosine nucleobase,
([PPG]) is a nucleoside comprising a 7-deaza-8-aza guanine nucleobase,
(G) is a nucleoside comprising a guanine nucleobase,
(U) is a nucleoside comprising a uracil nucleobase,
(T) is a nucleoside comprising a thymine nucleobase,
[LR] indicates that the following nucleoside is a beta-D-oxy-LNA nucleoside,
[dR] indicates that the following nucleoside is a DNA nucleoside,
[mR] indicates that the following nucleoside is a 2′-O-methyl RNA nucleoside,
[MOE] indicates that the following nucleoside is a 2′-O-methoxyethyl RNA (MOE RNA) nucleoside,
[sP] indicates a phosphorothioate internucleoside linkage between the preceding and following nucleoside,
[PS2] indicates a phosphorodithioate internucleoside linkage between the preceding and following nucleoside,
P indicates a phosphodiester internucleoside linkage between the preceding nucleoside or moiety and the following nucleoside, and
[5gn2c6] is an aminohexyl conjugated tri(N-acetyl-galactosamine) moiety as shown in FIG. 8.

In some embodiments, the antisense oligonucleotide conjugate comprises the following:

(SEQ ID NO 11, CMP ID NO 11_1)
[LR](G)[sP].[LR](T)[sP].[LR](T)[sP].[dR](T)[sP].[dR](T)[sP].[mR](A)[sP].[dR](A)[sP].[dR](G)
[sP].[dR](A)[sP].[dR](T)[sP].[dR](T)[sP].[dR](G)[sP].[dR](T)[sP].[dR](G)[sP].[dR](G)[sP].[dR]
(T)[sP].[dR](G)[sP].[LR](T)[sP].[LR](T)[PS2].[LR](T).

In some embodiments, the antisense oligonucleotide conjugate comprises the following:

(SEQ ID NO 11, CMP ID NO 11_1, CNJ ID NO 11_1)
[5gn2c6]P.[dR](C)P.[dR](A)P.[LR](G)[sP].[LR](T)[sP].[LR](T)[sP].[dR](T)[sP].[dR](T)[sP].[mR]
(A)[sP].[dR](A)[sP].[dR](G)[sP].[dR](A)[sP].[dR](T)[sP].[dR](T)[sP].[dR](G)[sP].[dR](T)[sP].
[dR](G)[sP].[dR](G)[sP].[dR](T)[sP].[dR](G)[sP].[LR](T)[sP].[LR](T)[PS2].[LR](T).

Targeting A1CF 38053-38071

The antisense oligonucleotide or antisense oligonucleotide conjugate of the invention may bind a target sequence within the region defined by positions 38053-38071 of the A1CF pre-mRNA (SEQ ID NO 45).

Thus, the invention provides an antisense oligonucleotide of 12 to 30 nucleotides in length, which comprises a contiguous nucleotide sequence of 12 to 30 nucleotides in length, wherein the contiguous nucleotide sequence is capable of binding to a target sequence in an A1CF mRNA, wherein the target sequence is a sequence of at least 17 contiguous nucleotides within CAGGUAUAUAACAAGUUCA (SEQ ID NO 34) (positions 38053-38071 of SEQ ID NO 45), and wherein the antisense oligonucleotide is capable of reducing A1CF expression.

The invention also provides an antisense oligonucleotide conjugate comprising an antisense oligonucleotide covalently attached to at least one conjugate moiety, wherein the antisense oligonucleotide is an antisense oligonucleotide of 12 to 30 nucleotides in length, which comprises a contiguous nucleotide sequence of 12 to 30 nucleotides in length, wherein the contiguous nucleotide sequence is capable of binding to a target sequence in an A1CF mRNA, wherein the target sequence is a sequence of at least 17 contiguous nucleotides within CAGGUAUAUAACAAGUUCA (SEQ ID NO 34) (positions 38053-38071 of SEQ ID NO 45), and wherein the antisense oligonucleotide is capable of reducing A1CF expression.

In some embodiments of the antisense oligonucleotide or the antisense oligonucleotide conjugate of the invention, the target sequence is a sequence of 17, 18, 19 or 20 contiguous nucleotides within SEQ ID NO 34.

In some embodiments, the target sequence is CAGGUAUAUAACAAGUUCA (SEQ ID NO 34) (positions 38053-38071 of SEQ ID NO 45).

In some embodiments, the sequence of the antisense oligonucleotide and/or the sequence of the contiguous nucleotide sequence comprises or consists of a sequence having at least 80% identity to TGAACTTGTTATATACETG (SEQ ID NO 4).

In some embodiments, the sequence of the antisense oligonucleotide and/or the sequence of the contiguous nucleotide sequence comprises or consists of a sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO 4.

In some embodiments, the sequence of the antisense oligonucleotide and/or the sequence of the contiguous nucleotide sequence comprises or consists of SEQ ID NO 4. In some embodiments, the sequence of the contiguous nucleotide sequence consists of SEQ ID NO 4. In some embodiments, the sequence of the antisense consists of SEQ ID NO 4.

In some embodiments, the antisense oligonucleotide comprises or consists of any one of CMP ID NOs 4_1, 4_2, or 4_3.

In some embodiments, the antisense oligonucleotide comprises CMP ID NO 4_1. In some embodiments, the antisense oligonucleotide consists of CMP ID NO 4_1.

In some embodiments, the antisense oligonucleotide comprises CMP ID NO 4_2. In some embodiments, the antisense oligonucleotide consists of CMP ID NO 4_2.

The invention provides an antisense oligonucleotide comprising or consisting of a compound selected from the following:

(a)
(SEQ ID NO 4, CMP ID NO 4_1)
[LR](T)[PS2].[LR](G)[sP].[LR](A)[sP].[dR](A)[sP].[dR](C)[sP].[dR](T)[sP].[dR](T)[sP].
[dR](G)[sP].[dR](T)[sP].[dR](T)[sP].[dR](A)[sP].[dR](T)[sP].[dR](A)[sP].[dR](T)[sP].
[dR](A)[sP].[dR](C)[sP].[LR]([5meC])[PS2].[LR](T)[PS2].[LR](G),
(b)
(SEQ ID NO 4, CMP ID NO 4_2)
[LR](T)[PS2].[LR](G)[sP].[LR](A)[sP].[mR](A)[sP].[dR](C)[sP].[dR](T)[sP].[dR](T)[sP].
[dR](G)[sP].[dR](T)[sP].[dR](T)[sP].[dR](A)[sP].[dR](T)[sP].[dR](A)[sP].[dR](T)[sP].
[dR](A)[sP].[dR](C)[sP].[LR]([5meC])[sP].[LR](T)[PS2].[LR](G),
and
(c)
(SEQ ID NO 4, CMP ID NO 4_3)
[LR](T)[PS2].[LR](G)[sP].[LR](A)[sP].[dR](A)[sP].[dR](C)[sP].[dR](T)[sP].[dR](T)[sP].
[dR](G)[sP].[dR](T)[sP].[dR](T)[sP].[dR](A)[sP].[dR](T)[sP].[dR](A)[sP].[dR](T)[sP].
[dR](A)[sP].[dR](C)[sP].[LR]([5meC])[P].[LR](T)[PS2].[LR](G),

wherein
(A) is a nucleoside comprising an adenine nucleobase,
(C) is a nucleoside comprising a cytosine nucleobase,
([5meC]) is a nucleoside comprising a 5-methyl cytosine nucleobase,
([PPG]) is a nucleoside comprising a 7-deaza-8-aza guanine nucleobase,
(G) is a nucleoside comprising a guanine nucleobase,
(U) is a nucleoside comprising a uracil nucleobase,
(T) is a nucleoside comprising a thymine nucleobase,
[LR] indicates that the following nucleoside is a beta-D-oxy-LNA nucleoside,
[dR] indicates that the following nucleoside is a DNA nucleoside,
[mR] indicates that the following nucleoside is a 2′-O-methyl RNA nucleoside,
[MOE] indicates that the following nucleoside is a 2′-O-methoxyethyl RNA (MOE RNA) nucleoside,
[sP] indicates a phosphorothioate internucleoside linkage between the preceding and following nucleoside, and
[PS2] indicates a phosphorodithioate internucleoside linkage between the preceding and following nucleoside.

In some embodiments, the antisense oligonucleotide comprises the following:

(SEQ ID NO 4, CMP ID NO 4_1)
[LR](T)[PS2].[LR](G)[sP].[LR](A)[sP].[dR](A)[sP].[dR](C)[sP].[dR](T)[sP].[dR](T)[sP].[dR](G)
[sP].[dR](T)[sP].[dR](T)[sP].[dR](A)[sP].[dR](T)[sP].[dR](A)[sP].[dR](T)[sP].[dR](A)[sP].[dR]
(C)[sP].[LR]([5meC])[PS2].[LR](T)[PS2].[LR](G).

In some embodiments the antisense oligonucleotide consists of the following:

(SEQ ID NO 4, CMP ID NO 4_1)
[LR](T)[PS2].[LR](G)[sP].[LR](A)[sP].[dR](A)[sP].[dR](C)[sP].[dR](T)[sP].[dR](T)[sP].[dR](G)
[sP].[dR](T)[sP].[dR](T)[sP].[dR](A)[sP].[dR](T)[sP].[dR](A)[sP].[dR](T)[sP].[dR](A)[sP].[dR]
(C)[sP].[LR]([5meC])[PS2].[LR](T)[PS2].[LR](G).

In some embodiments, the antisense oligonucleotide comprises the following:

(SEQ ID NO 4, CMP ID NO 4_2)
[LR](T)[PS2].[LR](G)[sP].[LR](A)[sP].[mR](A)[sP].[dR](C)[sP].[dR](T)[sP].[dR](T)[sP].[dR](G)
[sP].[dR](T)[sP].[dR](T)[sP].[dR](A)[sP].[dR](T)[sP].[dR](A)[sP].[dR](T)[sP].[dR](A)[sP].[dR]
(C)[sP].[LR]([5meC])[sP].[LR](T)[PS2].[LR](G).

In some embodiments, the antisense oligonucleotide consists of the following:

(SEQ ID NO 4, CMP ID NO 4_2)
[LR](T)[PS2].[LR](G)[sP].[LR](A)[sP].[mR](A)[sP].[dR](C)[sP].[dR](T)[sP].[dR](T)[sP].[dR](G)
[sP].[dR](T)[sP].[dR](T)[sP].[dR](A)[sP].[dR](T)[sP].[dR](A)[sP].[dR](T)[sP].[dR](A)[sP].[dR]
(C)[sP].[LR]([5meC])[sP].[LR](T)[PS2].[LR](G).

In some embodiments, the antisense oligonucleotide conjugate comprises or consists of any one of CNJ ID NOs 4_1, 4_2, or 4_3.

In some embodiments, the antisense oligonucleotide conjugate comprises CNJ ID NO 4_1. In some embodiments, the antisense oligonucleotide conjugate consists of CNJ ID NO 4_1.

In some embodiments, the antisense oligonucleotide conjugate comprises CNJ ID NO 4_2. In some embodiments, the antisense oligonucleotide conjugate consists of CNJ ID NO 4_2.

The invention provides an antisense oligonucleotide conjugate selected from the following:

(a)
(SEQ ID NO 4, CMP ID NO 4_1, CNJ ID NO 4_1)
[5gn2c6]P.[dR](C)P.[dR](A)P.[LR](T)[PS2].[LR](G)[P].[LR](A)[sP].[dR](A)[sP].[dR]
(C)[sP].[dR](T)[sP].[dR](T)[sP].[dR](G)[sP].[dR](T)[sP].[dR](T)[sP].[dR](A)[sP].[dR](T)
[sP].[dR](A)[sP].[dR](T)[sP].[dR](A)[sP].[dR](C)[sP].[LR]([5meC])[PS2].[LR](T)[PS2].
[LR](G),
(b)
(SEQ ID NO 4, CMP ID NO 4_2, CNJ ID NO 4_2)
[5gn2c6]P.[dR](C)P.[dR](A)P.[LR](T)[PS2].[LR](G)[sP].[LR](A)[sP].[mR](A)[sP].[dR]
(C)[sP].[dR](T)[sP].[dR](T)[sP].[dR](G)[sP].[dR](T)[sP].[dR](T)[sP].[dR](A)[sP].[dR](T)
[sP].[dR](A)[sP].[dR](T)[sP].[dR](A)[sP].[dR](C)[sP].[LR]([5meC])[sP].[LR](T)[PS2].
[LR](G),
and
(c)
(SEQ ID NO 4, CMP ID NO 4_3, CNJ ID NO 4_3)
[5gn2c6]P.[dR](C)P.[dR](A)P.[LR](T)[PS2].[LR](G)[P].[LR](A)[sP]. [dR](A)[sP].[dR](C)
[sP].[dR](T)[sP].[dR](T)[sP].[dR](G)[sP].[dR](T)[sP].[dR](T)[sP].[dR](A)[sP].[dR](T)[sP]
.[dR](A)[P].[dR](T)[sP].[dR](A)[sP].[dR](C)[sP].[LR]([5meC])[sP].[LR](T)[PS2].[LR]
(G),

wherein
(A) is a nucleoside comprising an adenine nucleobase,
(C) is a nucleoside comprising a cytosine nucleobase,
([5meC]) is a nucleoside comprising a 5-methyl cytosine nucleobase,
([PPG]) is a nucleoside comprising a 7-deaza-8-aza guanine nucleobase,
(G) is a nucleoside comprising a guanine nucleobase,
(U) is a nucleoside comprising a uracil nucleobase,
(T) is a nucleoside comprising a thymine nucleobase,
[LR] indicates that the following nucleoside is a beta-D-oxy-LNA nucleoside,
[dR] indicates that the following nucleoside is a DNA nucleoside,
[mR] indicates that the following nucleoside is a 2′-O-methyl RNA nucleoside,
[MOE] indicates that the following nucleoside is a 2′-O-methoxyethyl RNA (MOE RNA) nucleoside,
[sP] indicates a phosphorothioate internucleoside linkage between the preceding and following nucleoside,
[PS2] indicates a phosphorodithioate internucleoside linkage between the preceding and following nucleoside,
P indicates a phosphodiester internucleoside linkage between the preceding nucleoside or moiety and the following nucleoside, and
[5gn2c6] is an aminohexyl conjugated tri(N-acetyl-galactosamine) moiety as shown in FIG. 8.

In some embodiments, the antisense oligonucleotide conjugate comprises the following:

(SEQ ID NO 4, CMP ID NO 4_1, CNJ ID NO 4_1)
[5gn2c6]P.[dR](C)P.[dR](A)P.[LR](T)[PS2].[LR](G)[sP].[LR](A)[sP].[dR](A)[sP].[dR](C)[sP].
[dR](T)[sP].[dR](T)[sP].[dR](G)[sP].[dR](T)[sP].[dR](T)[sP].[dR](A)[sP].[dR](T)[P].[dR](A)
[sP][dR](T)[sP].[dR](A)[sP].[dR](C)[sP].[LR]([5meC])[PS2].[LR](T)[PS2].[LR](G).

In some embodiments, the antisense oligonucleotide conjugate consists of the following:

(SEQ ID NO 4, CMP ID NO 4_1, CNJ ID NO 4_1)
[5gn2c6]P.[dR](C)P.[dR](A)P.[LR](T)[PS2].[LR](G)[sP].[LR](A)[sP].[dR](A)[sP].[dR](C)[sP].
[dR](T)[sP].[dR](T)[sP].[dR](G)[sP].[dR](T)[sP].[dR](T)[sP].[dR](A)[sP].[dR](T)[sP].[dR](A)
[sP].[dR](T)[sP].[dR](A)[sP].[dR](C)[sP].[LR]([5meC])[PS2].[LR](T)[PS2].[LR](G).

In some embodiments, the antisense oligonucleotide conjugate comprises the following:

(SEQ ID NO 4, CMP ID NO 4_2, CNJ ID NO 4_2)
[5gn2c6]P.[dR](C)P.[dR](A)P.[LR](T)[PS2].[LR](G)[sP].[LR](A)[sP].[mR](A)[sP].[dR](C)[sP].
[dR](T)[sP].[dR](T)[P].[dR](G)[sP].[dR](T)[sP].[dR](T)[sP].[dR](A)[sP].[dR](T)[sP].[dR](A)[sP]
.[dR](T)[sP].[dR](A)[sP].[dR](C)[sP].[LR]([5meC])[sP].[LR](T)[PS2].[LR](G).

In some embodiments, the antisense oligonucleotide conjugate consist of the following:

(SEQ ID NO 4, CMP ID NO 4_2, CNJ ID NO 4_2)
[5gn2c6]P.[dR](C)P.[dR](A)P.[LR](T)[PS2].[LR](G)[sP].[LR](A)[sP].[mR](A)[sP].[dR](C)[sP].
[dR](T)[sP].[dR](T)[sP].[dR](G)[sP].[dR](T)[sP].[dR](T)[sP].[dR](A)[sP].[dR](T)[sP].[dR](A)
[P].[dR](T)[sP].[dR](A)[sP].[dR](C)[sP].[LR]([5meC])[sP].[LR](T)[PS2].[LR](G).

The invention provides an antisense oligonucleotide conjugate which is CNJ ID NO 4_1 as depicted in FIG. 4.

The invention provides an antisense oligonucleotide conjugate which is CNJ ID NO 4_2 as depicted in FIG. 5.

Targeting A1CF 78973-78990

The antisense oligonucleotide or antisense oligonucleotide conjugate of the invention may bind a target sequence within the region defined by positions 78973-78990 of the A1CF pre-mRNA (SEQ ID NO 45).

Thus, the invention provides an antisense oligonucleotide of 12 to 30 nucleotides in length, which comprises a contiguous nucleotide sequence of 12 to 30 nucleotides in length, wherein the contiguous nucleotide sequence is capable of binding to a target sequence in an A1CF mRNA, wherein the target sequence is a sequence of at least 17 contiguous nucleotides within AGACACACAAAACUCUAU (SEQ ID NO 44) (positions 78973-78990 of SEQ ID NO 45), and wherein the antisense oligonucleotide is capable of reducing A1CF expression.

The invention also provides an antisense oligonucleotide conjugate comprising an antisense oligonucleotide covalently attached to at least one conjugate moiety, wherein the antisense oligonucleotide is an antisense oligonucleotide of 12 to 30 nucleotides in length, which comprises a contiguous nucleotide sequence of 12 to 30 nucleotides in length, wherein the contiguous nucleotide sequence is capable of binding to a target sequence in an A1CF mRNA, wherein the target sequence is a sequence of at least 17 contiguous nucleotides within AGACACACAAAACUCUAU (SEQ ID NO 44) (positions 78973-78990 of SEQ ID NO 45), and wherein the antisense oligonucleotide is capable of reducing A1CF expression.

In some embodiments of the antisense oligonucleotide or the antisense oligonucleotide conjugate of the invention, the target sequence is a sequence of 17, 18, 19 or 20 contiguous nucleotides within SEQ ID NO 44.

In some embodiments, the target sequence is:

(SEQ ID NO 35)
AGACACACAAAACUCUA (positions 78973-78989 of SEQ ID NO 45),
or
(SEQ ID NO 40)
GACACACAAAACUCUAU (positions 78974-78990 of SEQ ID NO 45).

In some embodiments, the target sequence is AGACACACAAAACUCUA (SEQ ID NO 35) (positions 78973-78989 of SEQ ID NO 45).

In some embodiments, the sequence of the antisense oligonucleotide and/or the sequence of the contiguous nucleotide sequence comprises or consists of a sequence having at least 80% identity to any one of the following sequences:

(SEQ ID NO 5)
TAGAGTTTTGTGTGTET,
(SEQ ID NO 12)
TAGAGTTTUGTGTGTET,
(SEQ ID NO 13)
TAGAGTTTTGTGUGTET,
and
(SEQ ID NO 14)
ATAGAFTTTTGTGTGTE.

In some embodiments, the sequence of the antisense oligonucleotide and/or the sequence of the contiguous nucleotide sequence comprises or consists of a sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to any one of SEQ ID NOs 5, 12, 13 or 14.

In some embodiments, the sequence of the antisense oligonucleotide and/or the sequence of the contiguous nucleotide sequence comprises or consists of a sequence having at least 80% identity to SEQ ID NO 5.

In some embodiments, the sequence of the antisense oligonucleotide and/or the sequence of the contiguous nucleotide sequence comprises or consists of a sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO 5.

In some embodiments, the sequence of the antisense oligonucleotide and/or the sequence of the contiguous nucleotide sequence comprises or consists of SEQ ID NO 5. In some embodiments, the sequence of the sequence of the contiguous nucleotide sequence consists of SEQ ID NO 5. In some embodiments, the sequence of the antisense consists of SEQ ID NO 5.

In some embodiments, the antisense oligonucleotide comprises or consists of any one of CMP ID NOs 5_1, 5_2, 12_1, 5_3, 13_1 or 14_1.

In some embodiments, the antisense oligonucleotide comprises CMP ID NO 5_1. In some embodiments, the antisense oligonucleotide consists of CMP ID NO 5_1.

In some embodiments, the antisense oligonucleotide comprises CMP ID NO 5_2. In some embodiments, the antisense oligonucleotide consists of CMP ID NO 5_2.

The invention provides an antisense oligonucleotide comprising or consisting of a compound selected from the following:

(a)
(SEQ ID NO 5, CMP ID NO 5_1)
[LR](T)[PS2].[LR](A)[PS2].[dR](G)[sP].[dR](A)[sP].[LR](G)[sP].[dR](T)[sP].[dR](T)[sP].
[dR](T)[sP].[dR](T)[sP].[dR](G)[sP].[dR](T)[sP].[dR](G)[sP].[dR](T)[sP].[dR](G)[sP].
[dR](T)[sP].[MOE]([5meC])[sP].[MOE](T),
(b)
(SEQ ID NO 5, CMP ID NO 5_2)
[LR](T)[PS2].[LR](A)[sP].[dR](G)[sP].[dR](A)[P].[LR](G)[sP].[dR](T)[sP].[dR](T)[sP].
[dR](T)[sP].[dR](T)[sP].[dR](G)[sP].[dR](T)[sP].[dR](G)[sP].[dR](T)[sP].[dR](G)[sP].
[dR](T)[sP].[MOE]([5meC])[sP].[MOE](T),
(c)
(SEQ ID NO 12, CMP ID NO 12_1)
[LR](T)[PS2].[LR](A)[PS2].[dR](G)[sP].[dR](A)[sP].[LR](G)[sP].[dR](T)[sP].[dR](T)
[sP].[dR](T)[sP].[mR](U)[sP].[dR](G)[sP].[dR](T)[sP].[dR](G)[sP].[dR](T)[sP].[dR](G)
[sP].[dR](T)[sP].[MOE]([5meC])[sP].[MOE](T),
(d)
(SEQ ID NO 5, CMP ID NO 5_3)
[MOE](T)[sP].[MOE](A)[sP].[MOE](G)[sP].[MOE](A)[sP].[LR](G)[PS2].[dR](T)[sP].[dR]
(T)[sP].[dR](T)[sP].[dR](T)[sP].[dR](G)[sP].[dR](T)[sP].[dR](G)[sP].[dR](T)[sP].[dR](G)
[sP].[dR](T)[sP].[LR]([5meC])[PS2].[MOE](T),
(e)
(SEQ ID NO 13, CMP ID NO 13_1)
[MOE](T)[sP].[MOE](A)[sP].[MOE](G)[sP].[MOE](A)[sP].[LR](G)[PS2].[dR](T)[sP].[dR]
(T)[sP].[dR](T)[sP].[dR](T)[sP].[dR](G)[sP].[dR](T)[sP].[dR](G)[sP].[mR](U)[sP].[dR]
(G)[sP].[dR](T)[sP].[LR]([5meC])[PS2].[MOE](T),
and
(f)
(SEQ ID NO 14, CMP ID NO 14_1)
[LR](A)[PS2].[LR](T)[sP].[LR](A)[sP].[LR](G)[sP].[dR](A)[sP].[dR]([PPG])[sP].[dR](T)
[sP].[dR](T)[sP].[dR](T)[sP].[dR](T)[sP].[dR](G)[sP].[dR](T)[sP].[dR](G)[sP].[dR](T)
[sP].[dR](G)[sP].[LR](T)[PS2].[LR]([5meC]),

wherein
(A) is a nucleoside comprising an adenine nucleobase,
(C) is a nucleoside comprising a cytosine nucleobase,
([5meC]) is a nucleoside comprising a 5-methyl cytosine nucleobase,
([PPG]) is a nucleoside comprising a 7-deaza-8-aza guanine nucleobase,
(G) is a nucleoside comprising a guanine nucleobase,
(U) is a nucleoside comprising a uracil nucleobase,
(T) is a nucleoside comprising a thymine nucleobase,
[LR] indicates that the following nucleoside is a beta-D-oxy-LNA nucleoside,
[dR] indicates that the following nucleoside is a DNA nucleoside,
[mR] indicates that the following nucleoside is a 2′-O-methyl RNA nucleoside,
[MOE] indicates that the following nucleoside is a 2′-O-methoxyethyl RNA (MOE RNA) nucleoside,
[sP] indicates a phosphorothioate internucleoside linkage between the preceding and following nucleoside, and
[PS2] indicates a phosphorodithioate internucleoside linkage between the preceding and following nucleoside.

In some embodiments, the antisense oligonucleotide comprises the following compound:

(SEQ ID NO 5, CMP ID NO 5_1)
[LR](T)[PS2].[LR](A)[PS2].[dR](G)[sP].[dR](A)[sP].[LR](G)[sP].[dR](T)[P].[dR](T)[sP].[dR](T)
[sP].[dR](T)[sP].[dR](G)[sP].[dR](T)[sP].[dR](G)[sP].[dR](T)[sP].[dR](G)[sP].[dR](T)[sP].
[MOE]([5meC])[sP].[MOE](T).

In some embodiments, the antisense oligonucleotide consists of the following compound:

(SEQ ID NO 5, CMP ID NO 5_1)
[LR](T)[PS2].[LR](A)[PS2].[dR](G)[sP].[dR](A)[sP].[LR](G)[sP].[dR](T)[sP].[dR](T)[sP].[dR](T)
[sP].[dR](T)[sP].[dR](G)[sP].[dR](T)[sP].[dR](G)[sP].[dR](T)[sP].[dR](G)[sP].[dR](T)[sP].
[MOE]([5meC])[sP].[MOE](T).

In some embodiments, the antisense oligonucleotide comprises the following compound:

(SEQ ID NO 5, CMP ID NO 5_2)
[LR](T)[PS2].[LR](A)[sP].[dR](G)[sP].[dR](A)[sP].[LR](G)[sP].[dR](T)[sP].[dR](T)[P].[dR](T)
[sP].[dR](T)[sP].[dR](G)[sP].[dR](T)[sP].[dR](G)[sP].[dR](T)[sP].[dR](G)[sP].[dR](T)[sP].
[MOE]([5meC])[sP].[MOE](T).

In some embodiments, the antisense oligonucleotide consists of the following compound:

(SEQ ID NO 5, CMP ID NO 5_2)
[LR](T)[PS2].[LR](A)[sP].[dR](G)[sP].[dR](A)[sP].[LR](G)[sP].[dR](T)[sP].[dR](T)[sP].[dR](T)
[sP].[dR](T)[sP].[dR](G)[sP].[dR](T)[sP].[dR](G)[sP].[dR](T)[sP].[dR](G)[sP].[dR](T)[sP].
[MOE]([5meC])[sP].[MOE](T).

In some embodiments, the antisense oligonucleotide conjugate comprises or consists of any one of CNJ ID NOs 5_1, 5_2, 12_1, 5_3, 13_1 or 14_1.

In some embodiments, the antisense oligonucleotide conjugate comprises CNJ ID NO 5_1. In some embodiments, the antisense oligonucleotide conjugate consists of CNJ ID NO 5_1.

In some embodiments, the antisense oligonucleotide conjugate comprises CNJ ID NO 5_2. In some embodiments, the antisense oligonucleotide conjugate consists of CNJ ID NO 5_2.

The invention provides an antisense oligonucleotide conjugate selected from the following:

(a)
(SEQ ID NO 5, CMP ID NO 5_1, CNJ ID NO 5_1)
[5gn2c6]P.[dR](C)P.[dR](A)P.[LR](T)[PS2].[LR](A)[PS2].[dR](G)[sP].[dR](A)[sP].
[LR](G)[sP].[dR](T)[sP].[dR](T)[sP].[dR](T)[sP].[dR](T)[sP].[dR](G)[sP].[dR](T)[sP].
[dR](G)[sP].[dR](T)[sP].[dR](G)[sP].[dR](T)[sP].[MOE]([5meC])[sP].[MOE](T),
(b)
(SEQ ID NO 5, CMP ID NO 5_2, CNJ ID NO 5_2)
[5gn2c6]P.[dR](C)P.[dR](A)P.[LR](T)[PS2].[LR](A)[sP].[dR](G)[sP].[dR](A)[sP].[LR]
(G)[sP].[dR](T)[sP].[dR](T)[sP].[dR](T)[sP].[dR](T)[sP].[dR](G)[sP].[dR](T)[sP].[dR](G)
[sP].[dR](T)[sP].[dR](G)[sP].[dR](T)[sP].[MOE]([5meC])[sP].[MOE](T),
(c)
(SEQ ID NO 12, CMP ID NO 12_1, CNJ ID NO 12_1)
[5gn2c6]P.[dR](C)P.[dR](A)P.[LR](T)[PS2].[LR](A)[PS2].[dR](G)[sP].[dR](A)[sP].[LR]
(G)[sP].[dR](T)[sP].[dR](T)[sP].[dR](T)[sP].[mR](U)[sP].[dR](G)[sP].[dR](T)[sP].[dR]
(G)[sP].[dR](T)[sP].[dR](G)[sP].[dR](T)[sP].[MOE]([5meC])[sP].[MOE](T),
(d)
(SEQ ID NO 5, CMP ID NO 5_3, CNJ ID NO 5_3)
[5gn2c6]P.[dR](C)P.[dR](A)P.[MOE](T)[sP].[MOE](A)[sP].[MOE](G)[sP].[MOE](A)[sP]
[LR](G)[PS2].[dR](T)[sP].[dR](T)[sP].[dR](T)[sP].[dR](T)[sP].[dR](G)[sP].[dR](T)[sP].
[dR](G)[P].[dR](T)[sP].[dR](G)[sP].[dR](T)[sP].[LR]([5meC])[PS2].[MOE](T),
(e)
(SEQ ID NO 13, CMP ID NO 13_1, CNJ ID NO 13_1)
[5gn2c6]P.[dR](C)P.[dR](A)P.[MOE](T)[sP].[MOE](A)[sP].[MOE](G)[sP].[MOE](A)[sP]
.[LR](G)[PS2].[dR](T)[sP].[dR](T)[sP].[dR](T)[sP].[dR](T)[sP].[dR](G)[sP].[dR](T)[sP].
[dR](G)[sP].[mR](U)[sP].[dR](G)[sP].[dR](T)[sP].[LR]([5meC])[PS2].[MOE](T),
and
(f)
(SEQ ID NO 14, CMP ID NO 14_1, CNJ ID NO 14_1)
[5gn2c6]P.[dR](C)P.[dR](A)P.[LR](A)[PS2].[LR](T)[sP].[LR](A)[sP].[LR](G)[sP].[dR](A)
[sP].[dR]([PPG])[sP].[dR](T)[sP].[dR](T)[sP].[dR](T)[sP].[dR](T)[sP].[dR](G)[sP].[dR]
(T)[sP].[dR](G)[sP].[dR](T)[sP].[dR](G)[sP].[LR](T)[PS2].[LR]([5meC]),

wherein
(A) is a nucleoside comprising an adenine nucleobase,
(C) is a nucleoside comprising a cytosine nucleobase,
([5meC]) is a nucleoside comprising a 5-methyl cytosine nucleobase,
([PPG]) is a nucleoside comprising a 7-deaza-8-aza guanine nucleobase,
(G) is a nucleoside comprising a guanine nucleobase,
(U) is a nucleoside comprising a uracil nucleobase,
(T) is a nucleoside comprising a thymine nucleobase,
[LR] indicates that the following nucleoside is a beta-D-oxy-LNA nucleoside,
[dR] indicates that the following nucleoside is a DNA nucleoside,
[mR] indicates that the following nucleoside is a 2′-O-methyl RNA nucleoside,
[MOE] indicates that the following nucleoside is a 2′-O-methoxyethyl RNA (MOE RNA) nucleoside,
[sP] indicates a phosphorothioate internucleoside linkage between the preceding and following nucleoside,
[PS2] indicates a phosphorodithioate internucleoside linkage between the preceding and following nucleoside,
P indicates a phosphodiester internucleoside linkage between the preceding nucleoside or moiety and the following nucleoside, and
[5gn2c6] is an aminohexyl conjugated tri(N-acetyl-galactosamine) moiety as shown in FIG. 8.

In some embodiments, the antisense oligonucleotide comprises the following:

(SEQ ID NO 5, CMP ID NO 5_1, CNJ ID NO 5_1)
[5gn2c6]P.[dR](C)P.[dR](A)P.[LR](T)[PS2].[LR](A)[PS2].[dR](G)[sP].[dR](A)[sP].[LR](G)[sP].
[dR](T)[sP].[dR](T)[sP].[dR](T)[sP].[dR](T)[sP].[dR](G)[sP].[dR](T)[sP].[dR](G)[sP].[dR](T)
[sP].[dR](G)[sP].[dR](T)[sP].[MOE]([5meC])[sP].[MOE](T).

In some embodiments, the antisense oligonucleotide consists of the following:

(SEQ ID NO 5, CMP ID NO 5_1, CNJ ID NO 5_1)
[5gn2c6]P.[dR](C)P.[dR](A)P.[LR](T)[PS2].[LR](A)[PS2].[dR](G)[sP].[dR](A)[sP].[LR](G)[sP].
[dR](T)[sP].[dR](T)[sP].[dR](T)[sP].[dR](T)[sP].[dR](G)[sP].[dR](T)[sP].[dR](G)[sP].[dR](T)
[sP].[dR](G)[sP].[dR](T)[sP].[MOE]([5meC])[sP].[MOE](T).

In some embodiments, the antisense oligonucleotide comprises the following:

(SEQ ID NO 5, CMP ID NO 5_2, CNJ ID NO 5_2)
[5gn2c6]P.[dR](C)P.[dR](A)P.[LR](T)[PS2].[LR](A)[sP].[dR](G)[sP].[dR](A)[sP].[LR](G)[sP].
[dR](T)[sP].[dR](T)[sP].[dR](T)[sP].[dR](T)[sP].[dR](G)[sP].[dR](T)[sP].[dR](G)[sP].[dR](T)
[sP].[dR](G)[sP].[dR](T)[sP].[MOE]([5meC])[sP].[MOE](T).

In some embodiments, the antisense oligonucleotide consists of the following:

(SEQ ID NO 5, CMP ID NO 5_2, CNJ ID NO 5_2)
[5gn2c6]P.[dR](C)P.[dR](A)P.[LR](T)[PS2].[LR](A)[sP].[dR](G)[sP].[dR](A)[sP].[LR](G)[sP].
[dR](T)[sP].[dR](T)[sP].[dR](T)[sP].[dR](T)[sP].[dR](G)[sP].[dR](T)[sP].[dR](G)[sP].[dR](T)
[sP].[dR](G)[sP].[dR](T)[sP].[MOE]([5meC])[sP].[MOE](T).

The invention provides an antisense oligonucleotide conjugate which is CNJ ID NO 5_1 as depicted in FIG. 6.

The invention provides an antisense oligonucleotide conjugate which is CNJ ID NO 5_2 as depicted in FIG. 7.

Pharmaceutically Acceptable Salts

In some embodiments, the antisense oligonucleotide or the antisense oligonucleotide conjugate of the invention is in the form of a pharmaceutically acceptable salt.

The term “salt” as used herein conforms to its generally known meaning, i.e. an ionic assembly of anions and cations.

The term “pharmaceutically acceptable salts” refers to those salts, which retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid, particularly hydrochloric acid, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcystein. In addition, these salts may be prepared form addition of an inorganic base or an organic base to the free acid. Salts derived from an inorganic base include, but are not limited to, the sodium, potassium, lithium, ammonium, calcium, magnesium salts. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyamine resins. The compounds of the present invention can also be present in the form of zwitterions. Particularly preferred pharmaceutically acceptable salts of compounds of formula (I) are the salts of hydrochloric acid, hydrobromic acid, sulfuric acid, phosphoric acid and methanesulfonic acid.

In some embodiments, the antisense oligonucleotide is in the form of a pharmaceutically acceptable salt. In some embodiments, the antisense oligonucleotide conjugate is in the form of a pharmaceutically acceptable salt.

In some embodiments, the pharmaceutically acceptable salt is a sodium salt or a potassium salt. In some embodiments, the antisense oligonucleotide or the antisense oligonucleotide conjugate of the invention is in the form of a sodium salt. In some embodiments, the antisense oligonucleotide of the invention is in the form of a sodium salt. In some embodiments, the antisense oligonucleotide conjugate of the invention is in the form of a sodium salt. In some embodiments, the antisense oligonucleotide or the antisense oligonucleotide conjugate of the invention is in the form of a potassium salt. In some embodiments, the antisense oligonucleotide of the invention is in the form of a potassium salt. In some embodiments, the antisense oligonucleotide conjugate of the invention is in the form of a potassium salt.

Delivery of Antisense Oligonucleotides

In some embodiments, the antisense oligonucleotide or the antisense oligonucleotide conjugate of the invention is encapsulated in a lipid-based delivery vehicle, covalently linked to or encapsulated in a dendrimer, or conjugated to an aptamer. This may be for the purpose of delivering the antisense oligonucleotides of the invention to the targeted cells and/or to improve the pharmacokinetics of the antisense oligonucleotide. Examples of lipid-based delivery vehicles include oil-in-water emulsions, micelles, liposomes, and lipid nanoparticles.

In some embodiments, the antisense oligonucleotide is encapsulated in a lipid-based delivery vehicle. In some embodiments, the antisense oligonucleotide is covalently linked to a dendrimer. In some embodiments, the antisense oligonucleotide is encapsulated in a dendrimer. In some embodiments, the antisense oligonucleotide is conjugated to an aptamer.

In some embodiments, the antisense oligonucleotide conjugate is encapsulated in a lipid-based delivery vehicle. In some embodiments, the antisense oligonucleotide conjugate is covalently linked to a dendrimer. In some embodiments, the antisense oligonucleotide conjugate is encapsulated in a dendrimer. In some embodiments, the antisense oligonucleotide conjugate is conjugated to an aptamer.

Pharmaceutical Compositions

The invention provides a pharmaceutical composition comprising the antisense oligonucleotide or the antisense oligonucleotide conjugate of the invention, and a pharmaceutically acceptable diluent, solvent, carrier, salt and/or adjuvant. In some embodiments, the pharmaceutical composition comprises an aqueous diluent or solvent. In some embodiments, the aqueous diluent or solvent is phosphate buffered saline. In some embodiments, the aqueous diluent or solvent is sterile.

The invention provides a pharmaceutical composition comprising the antisense oligonucleotide of the invention, and a pharmaceutically acceptable salt. In some embodiments, the salt comprises a metal cation. In some embodiments, the pharmaceutically acceptable salt is selected from the group consisting of a sodium salt, a potassium salt and an ammonium salt.

The invention also provides a pharmaceutical solution of the antisense oligonucleotide of the invention or the conjugate thereof, wherein the pharmaceutical solution comprises the antisense oligonucleotide of the invention or the conjugate thereof and a pharmaceutically acceptable solvent, such as saline.

The invention also provides the antisense oligonucleotide of the invention or the conjugate thereof in solid powdered form, such as in the form of a lyophilized powder.

Method for Reducing A1CF Expression

The invention provides an in vitro method for reducing A1CF expression in a target cell, the method comprising administering an effective amount of the antisense oligonucleotide or the antisense oligonucleotide conjugate of the invention or the pharmaceutical composition of the invention to the target cell.

The invention provides an in vivo method for reducing A1CF expression in a target cell, the method comprising administering an effective amount of the antisense oligonucleotide or the antisense oligonucleotide conjugate of the invention or the pharmaceutical composition of the invention to the target cell.

In some embodiments, the target cell is an animal cell, preferably a mammalian cell such as a mouse cell, rat cell, hamster cell, or monkey cell. Most preferably the target cell is a human cell.

In some embodiments, the target cell is a hepatocyte.

In some embodiments, the target cell is infected with HBV. In some embodiments, the target cell is infected with HBV. In some embodiments, the target cell may comprise HBV cccDNA. In some embodiments, the target cell comprises A1CF pre-mRNA (such as SEQ ID NO 45), mature mRNA (such asd any of SEQ ID NOs 47 to 54) and HBV cccDNA.

In some embodiments, the target cell is an HBV infected primary human hepatocyte, either derived from an HBV infected individual or from a HBV infected mouse with a humanized liver (PhoenixBio, PXB-mouse).

In some embodiments of the in vivo method of the invention, the target cell is part of a subject suffering from HBV infection, such as chronic HBV infection. In some embodiments of the in vitro method of the invention, the target cell is derived from a subject suffering from HBV infection, such as chronic HBV infection.

In some embodiments of the method, A1CF expression is reduced compared to a control by at least 10%, at least 20%, at least 30%, at least 40%, at least 50%, at least 60%, at least 70%, at least 75%, at least 80%, at least 85%, at least 90%, at least 95% or 100%. In some embodiments, the control is a cell that has not been exposed to the antisense oligonucleotide or the antisense oligonucleotide conjugate. In some embodiments, A1CF expression is the amount of A1CF mRNA expressed by the target cell.

Methods of Treatment, Methods of Prevention and Medical Uses

The invention provides a method for treating a disease comprising administering a therapeutically effective amount of the antisense oligonucleotide, the antisense oligonucleotide conjugate or the pharmaceutical composition of the invention to a subject suffering from the disease.

The invention provides a method for treating a disease comprising administering a therapeutically effective amount of the antisense oligonucleotide of the invention to a subject suffering from the disease. The invention provides a method for treating a disease comprising administering a therapeutically effective amount of the antisense oligonucleotide conjugate of the invention to a subject suffering from the disease. The invention provides a method for treating a disease comprising administering a therapeutically effective amount of the pharmaceutical composition of the invention to a subject suffering from the disease.

The invention provides a method for preventing a disease comprising administering a prophylactically effective amount of the antisense oligonucleotide, the antisense oligonucleotide conjugate or the pharmaceutical composition of the invention to a subject susceptible to the disease.

The invention provides a method for preventing a disease comprising administering a prophylactically effective amount of the antisense oligonucleotide of the invention to a subject susceptible to the disease. The invention provides a method for preventing a disease comprising administering a prophylactically effective amount of the antisense oligonucleotide conjugate of the invention to a subject susceptible to the disease. The invention provides a method for preventing a disease comprising administering a prophylactically effective amount of the pharmaceutical composition of the invention to a subject susceptible to the disease.

The invention provides the antisense oligonucleotide, the antisense oligonucleotide conjugate or the pharmaceutical composition of the invention for use in the treatment of a disease in a subject.

The invention provides the antisense oligonucleotide of the invention for use in the treatment of a disease in a subject. The invention also provides the antisense oligonucleotide conjugate of the invention for use in the treatment of a disease in a subject. The invention also provides the pharmaceutical composition of the invention for use in the treatment of a disease in a subject.

The invention provides the antisense oligonucleotide, the antisense oligonucleotide conjugate or the pharmaceutical composition of the invention for use in the prevention of a disease in a subject.

The invention provides the antisense oligonucleotide of the invention for use in the prevention of a disease in a subject. The invention provides the antisense oligonucleotide conjugate for use in the prevention of a disease in a subject. The invention provides the pharmaceutical composition of the invention for use in the prevention of a disease in a subject.

The invention provides a use of the antisense oligonucleotide, the antisense oligonucleotide conjugate or the pharmaceutical composition of the invention for the preparation of a medicament for treatment of a disease in a subject.

The invention provides a use of the antisense oligonucleotide of the invention for the preparation of a medicament for treatment of a disease in a subject. The invention provides a use of the antisense oligonucleotide conjugate of the invention for the preparation of a medicament for treatment of a disease in a subject. The invention provides a use of the pharmaceutical composition of the invention for the preparation of a medicament for treatment of a disease in a subject.

The invention provides a use of the antisense oligonucleotide, the antisense oligonucleotide conjugate or the pharmaceutical composition of the invention for the preparation of a medicament for prevention of a disease in a subject.

The invention provides a use of the antisense oligonucleotide of the invention for the preparation of a medicament for prevention of a disease in a subject. The invention provides a use of the antisense oligonucleotide of the invention for the preparation of a medicament for prevention of a disease in a subject. The invention provides a use of the pharmaceutical composition of the invention for the preparation of a medicament for prevention of a disease in a subject.

The terms “treating”, “treatment” and “treats” as used herein refer to both treatment of an existing disease (e.g. a disease or disorder as herein referred to), or prevention of a disease, i.e. prophylaxis. It will therefore be recognized that treatment, as referred to herein may in some embodiments be prophylactic. Prophylactic can be understood as preventing an HBV infection from turning into a chronic HBV infection or the prevention of severe liver diseases such as liver cirrhosis and hepatocellular carcinoma caused by a chronic HBV infection.

Herein the term “preventing”, “prevention” or “prevents” relates to a prophylactic treatment, i.e. to a measure or procedure the purpose of which is to prevent, rather than to cure a disease.

Prevention means that a desired pharmacological and/or physiological effect is obtained that is prophylactic in terms of completely or partially preventing a disease or symptom thereof.

Accordingly, herein “preventing a HBV infection” includes preventing a HBV infection from occurring in a subject, and preventing the occurrence of symptoms of a HBV infection. In the present invention in particular the prevention of HBV infection in children from HBV infected mothers are contemplated. Also contemplated is the prevention of an acute HBV infection turning into a chronic HBV infection.

The invention provides for an antisense oligonucleotide of the invention, an antisense oligonucleotide conjugate of the invention or a pharmaceutical composition of the invention, for use as a medicament. The invention provides an antisense oligonucleotide of the invention, an antisense oligonucleotide conjugate of the invention or a pharmaceutical composition of the invention for use in therapy. The invention provides for an antisense oligonucleotide of the invention, an antisense oligonucleotide conjugate of the invention or a pharmaceutical composition of the invention, for the preparation of a medicament.

Disease

In some embodiments, the disease is HBV infection. In some embodiments, the HBV infection is chronic HBV infection.

The term “hepatitis B virus infection” or “HBV infection” is commonly known in the art and refers to an infectious disease that is caused by the hepatitis B virus (HBV) and affects the liver. A HBV infection can be an acute or a chronic infection. Chronic hepatitis B virus (CHB) infection is a global disease burden affecting 248 million individuals worldwide. Approximately 686,000 deaths annually are attributed to HBV-related end-stage liver diseases and hepatocellular carcinoma (HCC) (GBD 2013; Schweitzer et al., 2015). WHO projected that without expanded intervention, the number of people living with CHB infection will remain at the current high levels for the next 40-50 years, with a cumulative 20 million deaths occurring between 2015 and 2030 (WHO 2016). CHB infection is not a homogenous disease with singular clinical presentation. Infected individuals have progressed through several phases of CHB-associated liver disease in their life; these phases of disease are also the basis for treatment with standard of care (SOC). Current guidelines recommend treating only selected CHB-infected individuals based on three criteria—serum ALT level, HBV DNA level, and severity of liver disease (EASL, 2017). This recommendation was due to the fact that SOC i.e. nucleos(t)ide analogs (NAs) and pegylated interferon-alpha (PEG-IFN), are not curative and must be administered for long periods of time thereby increasing their safety risks. PEG-IFN can only be administered to a small subset of CHB due to its various side effects. NAs effectively suppress HBV DNA replication; however, they have very limited/no effect on other viral markers. Two hallmarks of HBV infection, hepatitis B surface antigen (HBsAg) and covalently closed circular DNA (cccDNA) are the main targets of novel drugs aiming for HBV cure. In the plasma of CHB individuals, HBsAg subviral (empty) particles outnumber HBV virions by a factor of 103 to 105 (Ganem & Prince, 2014); its excess is believed to contribute to immunopathogenesis of the disease, including inability of individuals to develop neutralizing anti-HBs antibody, the serological marker observed following resolution of acute HBV

In some embodiments, the disease is associated with HBV infection. In some embodiments, the disease is a liver disease. In some embodiments, the disease is liver cirrhosis. In some embodiments, the disease is hepatocellular carcinoma.

Subject

For the purposes of the present invention, the “subject” or “patient” may be a vertebrate. In context of the present invention, the term “subject” includes both humans and other animals, particularly mammals, and other organisms. Thus, the herein provided means and methods are applicable to both human therapy and veterinary applications. Accordingly, herein the subject may be an animal such as a mouse, rat, hamster, rabbit, guinea pig, ferret, cat, dog, chicken, sheep, bovine species, horse, camel, or primate. Preferably, the subject is a mammal. More preferably, the subject is human. In some embodiments, the subject is suffering from a disease as referred to herein, such as HBV infection. In some embodiments, the subject is susceptible to said disease.

Administration

The antisense oligonucleotide of the invention, the antisense oligonucleotide conjugate of the invention or the pharmaceutical composition of the invention may be administered topically (such as, to the skin, inhalation, ophthalmic or otic) or enteral (such as, orally or through the gastrointestinal tract) or parenterally (such as, intravenous, subcutaneous, intra-muscular, intracerebral, intracerebroventricular or intrathecal).

In a preferred embodiment the antisense oligonucleotide of the invention, the antisense oligonucleotide conjugate of the invention or pharmaceutical composition of the invention is administered by a parenteral route including intravenous, intraarterial, subcutaneous, intraperitoneal or intramuscular injection or infusion, intrathecal or intracranial, e.g., intracerebral or intraventricular, administration. In one embodiment the antisense oligonucleotide of the invention is administered intracerebrally or intracerebroventricularly. In another embodiment the antisense oligonucleotide of the invention is administered intrathecally.

The invention also provides for the use of the antisense oligonucleotide of the invention or pharmaceutical composition of the invention as described for the preparation of a medicament wherein the medicament is in a dosage form for intrathecal administration.

The invention also provides for the use of the antisense oligonucleotide of the invention or pharmaceutical composition of the invention as described for the preparation of a medicament wherein the medicament is in a dosage form for intraventricular administration.

In some embodiments, the antisense oligonucleotide, the antisense oligonucleotide conjugate or the pharmaceutical composition of the invention is for use in a combination treatment with another therapeutic agent.

HELM Notation

Antisense oligonucleotides (compounds) of the invention and antisense oligonucleotide conjugates (conjugates) of the invention are depicted herein using Hierarchical Editing Language for Macromolecules (HELM) notation.

HELM is a notation format designed to depict the structure of macromolecules. Full details of HELM notation may be found at www.pistoiaalliance.org/helm-tools/, in Zhang et al. J. Chem. Inf. Model. 2012, 52, 2796-2806 (which initially described HELM notation) and in Milton et al. J. Chem Inf. Model. 2017, 57, 1233-1239 (which describes HELM version 2.0).

Briefly, a macromolecule is depicted as a “HELM string”, which is divided into sections. The first section lists the molecules comprised in the macromolecule. The second section lists the connections between molecules within the macromolecule. One or more dollar sign $ marks the end of a section of a HELM string.

Compounds of the invention are represented by a HELM string consisting of a single first section defining the oligonucleotide.

Conjugates of the invention are represented by a HELM string consisting of two sections: a first section, which defines the oligonucleotide (including a 5′ CA dinucleotide linker) and the conjugate moiety, and a second section, which defines the connection between the oligonucleotide and the conjugate moiety. Third, fourth and fifth sections (which may be used in HELM strings for more complex macromolecules) are not used in the HELM strings herein.

Each molecule listed in the first section of a HELM string is given an identifier (e.g. “RNA1” for a nucleic acid, “PEPTIDE1” for an amino acid sequence, “CHEM1” for a chemical structure) and the structure of the molecule is defined by notation in braces { } immediately following the identifier. The HELM notations used to define the structure of each molecule in braces { } in the first section of HELM strings for the compounds and conjugates of the present invention are as follows:
[LR](G) is a beta-D-oxy-LNA guanine nucleoside,
[LR](T) is a beta-D-oxy-LNA thymine nucleoside,
[LR](A) is a beta-D-oxy-LNA adenine nucleoside,
[LR]([5meC]) is a beta-D-oxy-LNA 5-methyl cytosine nucleoside,
[dR](G) is a DNA guanine nucleoside,
[dR](T) is a DNA thymine nucleoside,
[dR](A) is a DNA adenine nucleoside,
[dR]([C]) is a DNA cytosine nucleoside,
[mR](G) is a 2′-O-methyl RNA guanine nucleoside,
[mR](U) is a 2′-O-methyl RNA uracil nucleoside,
[mR](A) is a 2′-O-methyl RNA adenine nucleoside,
[mR]([C]) is a 2′-O-methyl RNA cytosine nucleoside,
[sP] is a phosphorothioate internucleoside linkage,
[PS2] is a phosphorodithioate internucleoside linakge
[MOE](G) is a 2′-MOE RNA guanine nucleoside,
[MOE](T) is a 2′-MOE RNA thymine nucleoside,
[MOE](A) is a 2′-MOE thyl RNA adenine nucleoside,
[MOE]([5meC]) is a 2′-MOE RNA 5-methyl cytosine nucleoside,
[dR]([PPG]) is a DNA 7-deaza-8-aza guanine nucleoside,
[P] is a phosphodiester internucleoside linkage,
. is used to demarcate nucleosides, and
[5gn2c6] is an aminohexyl conjugated tri(N-acetyl-galactosamine) moiety as shown in FIG. 8.

As noted above, in the context of the invention, a second section is used only in HELM strings representing conjugates of the invention. This second section lists the connections between the molecules listed in the first section. Each pair of molecules that are connected are defined by listing their identifiers, and then the attachment points between them (i.e. the point at which there is a covalent bond between the molecules) are defined.

In HELM strings representing the conjugates of the invention there is a single connection (between the conjugate moiety and the oligonucleotide). This single connection is represented in all HELM strings herein as follows:
CHEM1,RNA1,1:R2-1:R1.

This indicates that the conjugate moiety (CHEM1) is attached to the oligonucleotide (RNA1) by a covalent bond between the R2 attachment point of the first monomer of CHEM1 (which is the entire conjugate moiety as depicted in FIG. 8) (indicated by “1:R2”) and the R1 attachment point of the first monomer of RNA1 (indicated by “1:R1”).

Examples of HELM Notation

For example, CMP ID NO 1_1 is represented by the following HELM string (as depicted in Table 4):

RNA1{[LR](A)[sP].[LR](G)[sP].[dR](G)[sP].[dR](C)[sP].[LR](A)[sP].[dR](T)[sP].[dR](T)[
sP].[dR](A)[sP].[dR](T)[sP].[dR](A)[sP].[dR](G)[sP].[dR](G)[sP].[dR](T)[sP].[dR](T)[sP]
.[dR](T)[sP].[LR](T)[sP].[LR](T)[sP].[LR]([5mec])}$$$$V2.0

This HELM string consists of a single section listing the oligonucleotide of CMP ID NO 1_1. The initial “RNA1” indicates the molecule is a nucleic acid (oligonucleotide). The structure of the oligonucleotide is presented using HELM notation in the braces { } following RNA1. “$$$$” marks the end of the section, and of the HELM string as a whole. “V2.0” indicates that HELM version 2.0 is used.

As another example, CNJ ID NO 1_1 is represented by the following HELM string (as depicted in Table 5):

CHEM1{[5gn2c6]}|RNA1{P.[dR](C)P.[dR](A)P.[LR](G)[sP].[LR]([5meC])[sP].[dR](A)[s
P].[dR](T)[sP].[dR](T)[sP].[dR](A)[sP].[dR](T)[sP].[dR](A)[sP].[dR](G)[sP].[dR](G)[sP].[
dR](T)[sP].[dR](T)[sP].[dR](T)[sP].[LR](T)[sP].[LR](T)[sP].[LR]([5meC])[sP].[LR](T)}$C
HEM1, RNA1, 1:R2-1:R1$$$V2.0

This HELM string consists of two sections. The first section lists the conjugate moiety (which has the identifier CHEM1) and the oligonucleotide (identifier RNA1) which are the two molecules which constitute the antisense oligonucleotide conjugate of CNJ ID NO 1_1. The conjugate moiety is defined in braces { } immediately after CHEM1, and the oligonucleotide (including CA dinucleotide linker) is defined in braces { } after RNA1. “$” marks the end of the first section. The second section defines how CHEM1 and RNA1 are connected to one another. “CHEM1,RNA1,1:R2-1:R1” indicates that the R2 attachment point of the conjugate moiety (1:R2) is attached to the R1 attachment point of the first monomer of the oligonucleotide (in this case, a phosphate group [P]). “$$$” marks the end of the second section, and of the HELM string as a whole. “V2.0” again indicates that HELM version 2.0 is used.

Simplified HELM Notation

A simplified HELM notation is also used herein to depict the compounds and conjugates of the invention in a more readable format than a typical HELM string. In this simplified notation, the second section (listing connection between molecules) is omitted. In each HELM string herein depicting a conjugate of the invention, the connection between the conjugate moiety and the oligonucleotide is a phosphodiester bond between the conjugate moiety and the 5′ nucleoside of the oligonucleotide depicted. The “CHEM1” and “RNA1” identifiers and associated braces { }, the section end $, and the HELM version number are also omitted in the simplified HELM notation.

For example, CMP ID NO 1_1 is represented herein in simplified HELM notation as follows:

[LR](A)[sP].[LR](G)[sP].[dR](G)[sP].[dR](C)[sP].[LR](A)[sP].[dR](T)[sP].[dR](T)[sP].[d
R](A)[sP].[dR](T)[sP].[dR](A)[sP].[dR](G)[sP].[dR](G)[sP].[dR](T)[sP].[dR](T)[sP].[dR]
(T)[sP].[LR](T)[sP].[LR](T)[sP].[LR]([5meC])

For example, CNJ ID NO 1_1 is represented herein in simplified HELM notation as follows:

[5gn2c6]P.[dR](C)P.[dR](A)P.[LR](A)[sP].[LR](G)[sP].[dR](G)[sP].[dR](C)[sP].[LR]
(A)[sP].[dR](T)[sP].[dR](T)[sP].[dR](A)[P].[dR](T)[sP].[dR](A)[sP].[dR](G)[sP].[dR](G)
[sP].[dR](T)[sP].[dR](T)[sP].[dR](T)[sP].[LR](T)[sP].[LR](T)[sP].[LR]([5meC])

In all simplified HELM strings herein, [5gn2c6]P. indicates the conjugate moiety as depicted in FIG. 8 attached by a phosphodiester bond to the oligonucleotide represented by the rest of the HELM string.

It will be understood that a simplified HELM string represents the same molecule as the corresponding full HELM string. Thus, the simplified HELM string for a given compound or conjugate is interchangeable with the full HELM string for that compound or conjugate (as depicted in Tables 4 and 5 herein).

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology, biochemistry, cell biology, virology or related fields are intended to be within the scope of the following claims.

### EXAMPLES

### Example 1—Antisense Oligonucleotide Sequences, Compounds and Conjugates

68 antisense oligonucleotides (compounds) targeting A1CF mRNA were synthesised. Conjugate forms of these 68 antisense oligonucleotides, wherein an aminohexyl conjugated tri(N-acetyl-galactosamine) conjugate moiety (as depicted in FIG. 8) was covalently attached to the 5′ end of each antisense oligonucleotide via a CA dinucleotide linker, were also synthesised.

Oligonucleotide Synthesis

Oligonucleotide synthesis is generally known in the art. The antisense oligonucleotides and conjugates of the invention may be synthesized by any such method known in the art. Below is a protocol which may be applied. The oligonucleotides of the present invention may have been produced by slightly varying methods in terms of apparatus, support and concentrations used.

Oligonucleotides are synthesized on uridine universal supports using the phosphoramidite approach on a Mermade 192 at 1 μmol scale. At the end of the synthesis, the oligonucleotides are cleaved from the solid support using aqueous ammonia for 5-16 hours at 60° C. The oligonucleotides are purified by reverse phase HPLC (RP-HPLC), ion exchange chromatography or by solid phase extractions and characterized by UPLC, and the molecular mass is further confirmed by ESI-MS.

Elongation of the Oligonucleotide:

The coupling of β-cyanoethyl-phosphoramidites (DNA-A(Bz), DNA-G(ibu), DNA-C(Bz), DNA-T, LNA-5-methyl-C(Bz), LNA-A(Bz), LNA-G(dmf), or LNA-T) is performed by using a solution of 0.1 M of the 5′-O-DMT-protected amidite in acetonitrile and DCI (4,5-dicyanoimidazole) in acetonitrile (0.25 M) as activator. For the final cycle a phosphoramidite with desired modifications can be used, e.g. a C6 linker for attaching a conjugate group or a conjugate group as such. Thiolation for introduction of phosphorthioate linkages is carried out by using xanthane hydride (0.01 M in acetonitrile/pyridine 9:1). Phosphordiester linkages can be introduced using 0.02 M iodine in THF/Pyridine/water 7:2:1. The rest of the reagents are the ones typically used for oligonucleotide synthesis.

For post solid phase synthesis conjugation a commercially available C6 aminolinker phorphoramidite can be used in the last cycle of the solid phase synthesis and after deprotection and cleavage from the solid support the aminolinked deprotected oligonucleotide is isolated. The conjugates are introduced via activation of the functional group using standard synthesis methods.

Purification by RP-HPLC:

The crude compounds are purified by preparative RP-HPLC on a Phenomenex Jupiter® C18 10 μm 150×10 mm column. 0.1 M ammonium acetate pH 8 and acetonitrile is used as buffers at a flow rate of 5 mL/min. The collected fractions are lyophilized to give the purified compound typically as a white solid.

### Abbreviations

DCI: 4,5-Dicyanoimidazole
DCM: Dichloromethane
DMF: Dimethylformamide
DMT: 4,4′-Dimethoxytrityl
THF: Tetrahydrofurane
Bz: Benzoyl
lbu: Isobutyryl
RP-HPLC: Reverse phase high performance liquid chromatography

Tables of Sequences, Compounds and Conjugates

Table 3 below shows the sequences of the antisense oligonucleotides (ASOs), alongside the pattern of sugar moieties and internucleoside linkages therein, and with reference to the corresponding target sequence in A1CF mRNA. The code used in Table 3 is as follows:
Code for nucleobases: A=adenine, C=cytosine, E=5-methyl cytosine, F=7-deaza-8-azaguanine, G=guanine, T=thymine, U=uracil
Code for sugars: D=DNA, L=LNA, M=2′-O-methoxyethyl-RNA (MOE), O=2′-O-methyl-RNA
Code for internucleoside linkages: S=phosphorothioate, 2=phosphorodithioate

Table 4 below represents the compounds using HELM strings. Likewise, Table 5 below represents the conjugates using HELM strings. Details regarding how to read a HELM string are provided at www.pistoiaalliance.org/helm-tools/ and Zhang et al. J. Chem. Inf. Model. 2012, 52, 2796-2806.

HELM Annotation Key:
RNA1 is the identifier of the oligonucleotide part of the compound or conjugate; the oligonucleotide is defined in braces { } after the first instance of “RNA1”; the second instance of “RNA1” in each conjugate string is defining the location of the link between the oligonucleotide and the conjugate moiety,
CHEM1 is the identifier of the conjugate moiety part of the conjugate; the conjugate moiety is defined in braces { } after the first instance of “CHEM1”; the second instance of “CHEM1” in each conjugate string is defining the location of the link between the oligonucleotide and the conjugate moiety,
one or more $ marks the end of a section of a HELM string; the first section of HELM string defines the oligonucleotide sequence (including sugar moieties and internucleoside linkages); the second section (present only in the conjugate HELM strings) defines the location of the link between the oligonucleotide and conjugate moiety,
V2.0 indicates that HELM version 2.0 is used,
[LR](G) is a beta-D-oxy-LNA guanine nucleoside,
[LR](T) is a beta-D-oxy-LNA thymine nucleoside,
[LR](A) is a beta-D-oxy-LNA adenine nucleoside,
[LR]([5meC]) is a beta-D-oxy-LNA 5-methyl cytosine nucleoside,
[dR](G) is a DNA guanine nucleoside,
[dR](T) is a DNA thymine nucleoside,
[dR](A) is a DNA adenine nucleoside,
[dR]([C]) is a DNA cytosine nucleoside,
[mR](G) is a 2′-O-methyl RNA guanine nucleoside,
[mR](U) is a 2′-O-methyl RNA uracil nucleoside,
[mR](A) is a 2′-O-methyl RNA adenine nucleoside,
[mR]([C]) is a 2′-O-methyl RNA cytosine nucleoside,
[sP] is a phosphorothioate internucleoside linkage,
[PS2] is a phosphorodithioate internucleoside linakge
[MOE](G) is a 2′-MOE RNA guanine nucleoside,
[MOE](T) is a 2′-MOE RNA thymine nucleoside,
[MOE](A) is a 2′-MOE thyl RNA adenine nucleoside,
[MOE]([5meC]) is a 2′-MOE RNA 5-methyl cytosine nucleoside,
[dR]([PPG]) is a DNA 7-deaza-8-aza guanine nucleoside,
[P] is a phosphodiester internucleoside linkage
. is used to demarcate nucleosides, and
[5gn2c6] is a aminohexyl conjugated tri(N-acetyl-galactosamine) moiety as shown in FIG. 8.

Each of the conjugates of the invention set out in Table 5 consists of an ASO corresponding to the indicated CMP ID NO covalently attached at its 5′ end to an aminohexyl conjugated tri(N-acetyl-galactosamine) moiety as shown in FIG. 8 via a dinucleotide (CA) linker, wherein the nucleosides of the dinucleotide linker are DNA nucleosides which are linked to one another, to the ASO and to the conjugate via phosphodiester internucleoside linkages.

TABLE 3
Sequences and corresponding target sequences

| ASO | | | | | |
|---|---|---|---|---|---|
| SEQ | | | ASO | Target | Position in |
| ID | ASO nucleobase | ASO sugar | internuceloside | SEQ | A1CF gene |
| NO | sequence | sequence | linkages | ID NO | (SEQ ID NO 45) |
| 1 | AGGCATTATAGGTTTTTE | LLDDLDDDDDDDDDDLLL | SSSSSSSSSSSSSSSSS | 31 | 6953-6970 |
| 2 | AAAAGTTTCTCAGATAGGE | LLDOLDDDDDDDDDDDOLL | 2SSSSSSSSSSSSSSSS2 | 32 | 2181-2199 |
| 3 | GGACATGTTAATTTTACTT | LLODDDLDDDDDDDDDDLL | SSSSSSSSSSSSSSSSS2 | 33 | 16970-16988 |
| 4 | TGAACTTGTTATATACETG | LLLDDDDDDDDDDDDDLLL | 2SSSSSSSSSSSSSSS22 | 34 | 38053-38071 |
| 4 | TGAACTTGTTATATACETG | LLLODDDDDDDDDDDDLLL | 2SSSSSSSSSSSSSSSS2 | 34 | 38053-38071 |
| 5 | TAGAGTTTTGTGTGTET | LLDDLDDDDDDDDDDMM | 22SSSSSSSSSSSSSS | 35 | 78973-78989 |
| 5 | TAGAGTTTTGTGTGTET | LLDDLDDDDDDDDDDMM | 2SSSSSSSSSSSSSSS | 35 | 78973-78989 |
| 6 | GEATTATAGGTTTTTET | LLDDDDDDDDDDDLLLL | SSSSSSSSSSssSSSS | 36 | 6952-6968 |
| 7 | GGCATTATAGGTTTTTE | MLDLDDDDDDDDDLMMM | SSSSSSssSSSSSSSS | 37 | 6953-6969 |
| 7 | GGCATTATAGGTTTTTE | LLDLDDDDODDDDDLLL | SSSSSSSSSSSSSSSS | 37 | 6953-6969 |
| 8 | GGEATTATAGGTTTTTE | MMMLDDDDODDDDLLLM | SSS2SSSSSSSSS222 | 37 | 6953-6969 |
| 2 | AAAAGTTTCTCAGATAGGE | LLDOLDDDDDDDDDDDDLL | 22SSSSSSSSSSSSSSS2 | 32 | 2181-2199 |
| 9 | TGGACAUGTTAATTTTAE | LLDLDDODDLDDDDDDLL | 2SSSSSSSSSSSSSSS2 | 38 | 16972-16989 |
| 10 | GGACATGUTAATTTTACTT | LLDDDDLODODDDDDDDLL | 22SSSSSSSSSSSSSSS2 | 33 | 16970-16988 |
| 11 | GTTTTAAGATTGTGGTGTTT | LLLDDODDDDDDDDDDDLLL | SSSSSSSSSSSSSSSSSS2 | 39 | 26358-26377 |
| 4 | TGAACTTGTTATATACETG | LLLDDDDDDDDDDDDDLLL | 2SSSSSSSSSSSSSSSS2 | 34 | 38053-38071 |
| 12 | TAGAGTTTUGTGTGTET | LLDDLDDDODDDDDDMM | 22SSSSSSSSSSSSSS | 35 | 78973-78989 |
| 5 | TAGAGTTTTGTGTGTET | MMMMLDDDDDDDDDDLM | SSSS2SSSSSSSSSS2 | 35 | 78973-78989 |
| 13 | TAGAGTTTTGTGUGTET | MMMMLDDDDDDDODDLM | SSSS2SSSSSSSSSS2 | 35 | 78973-78989 |
| 14 | ATAGAFTTTTGTGTGTE | LLLLDDDDDDDDDDDLL | 2SSSSSSSSSSSSSS2 | 40 | 78974-78990 |
| 15 | AAAAFTTTCTCAGATAGGE | LLLDDDDDDDDDDDDDLLL | 2SSSSSSSSSSSSSSSS2 | 32 | 2181-2199 |
| 16 | AAAAGTTTCTCAFATAGGE | LLLDDDDDDDDDDDDDLLL | 2SSSSSSSSSSSSSSSS2 | 32 | 2181-2199 |
| 16 | AAAAGTTTCTCAFATAGGE | MLLDDDDDDDDDDDDDLLM | S2SSSSSSSSSSSSSSS2 | 32 | 2181-2199 |
| 16 | AAAAGTTTCTCAFATAGGE | MLLDDDDDDDDDDDDDLLM | S2SSSSSSSSSSSSSS22 | 32 | 2181-2199 |
| 2 | AAAAGTTTCTCAGATAGGE | LLDOLDDDDDDDDDDDDLL | 2SSSSSSSSSSSSSSSS2 | 32 | 2181-2199 |
| 2 | AAAAGTTTCTCAGATAGGE | LLDOLDDDDDDDDDDDOLL | 22SSSSSSSSSSSSSSS2 | 32 | 2181-2199 |
| 2 | AAAAGTTTCTCAGATAGGE | LLLDDDDDDDDODDDDLLL | 2SSSSSSSSSSSSSSSS2 | 32 | 2181-2199 |
| 2 | AAAAGTTTCTCAGATAGGE | LMMDDDDDDDDODDDDLLM | 2SSSSSSSSSSSSSSS22 | 32 | 2181-2199 |
| 2 | AAAAGTTTCTCAGATAGGE | LLLDDDDDDDODDDDDLLL | 2SSSSSSSSSSSSSSSS2 | 32 | 2181-2199 |
| 2 | AAAAGTTTCTCAGATAGGE | MMLDDDDDDDODDDDDLLM | SS2SSSSSSSSSSSSSS2 | 32 | 2181-2199 |
| 2 | AAAAGTTTCTCAGATAGGE | MLLDDDDDDDDODDDDLLM | SSSSSSSSSSSSSSSSSS | 32 | 2181-2199 |
| 2 | AAAAGTTTCTCAGATAGGE | LLLDDDDDODDDDDDDLLL | 2SSSSSSSSSSSSSSSS2 | 32 | 2181-2199 |
| 2 | AAAAGTTTCTCAGATAGGE | MLLDDDDDODDDDDDDLLM | SSSSSSSSSSSSSSSSS2 | 32 | 2181-2199 |
| 2 | AAAAGTTTCTCAGATAGGE | MLLDDDDDDDDDDDDOLLM | S2SSSSSSSSSSSSSSS2 | 32 | 2181-2199 |
| 17 | AAAAGTTUCTCAGATAGGE | MMLDDDDODDDDDDDDLLM | SSSSSSSSSSSSSSSSS2 | 32 | 2181-2199 |
| 18 | AGGCATTATAGGUTTTTE | LLDDLDDDDDDDODDLLL | 2SSSSSSSSSSSSSSS2 | 31 | 6953-6970 |
| 19 | AGGCAUTATAGGTTTTTE | LLDDLODDDDDDDDDLLL | 2SSSSSSSSSSSSSSS2 | 31 | 6953-6970 |
| 20 | GEATTATAGGTTTTTCTE | LLDDDDDDDDDLDDDDLL | 2SSSSSSSSSSSSSSS2 | 41 | 6951-6968 |
| 20 | GEATTATAGGTTTTTCTE | LLODDDDDDDDLDDDDLL | 2SSSSSSSSSSSSSSS2 | 41 | 6951-6968 |
| 20 | GEATTATAGGTTTTTCTE | LLDDDDDODDDLDDDDLL | 2SSSSSSSSSSSSSSS2 | 41 | 6951-6968 |
| 20 | GEATTATAGGTTTTTCTE | LLDDDDDDODDLDDDDLL | 2SSSSSSSSSSSSSSS2 | 41 | 6951-6968 |
| 20 | GEATTATAGGTTTTTCTE | LLDDDDDDDDDLDDDDLM | 2SSSSSSSSSSSSSSS2 | 41 | 6951-6968 |
| 20 | GEATTATAGGTTTTTCTE | LLDDDDDDDODLDDDDLL | 2SSSSSSSSSSSSSSS2 | 41 | 6951-6968 |
| 6 | GEATTATAGGTTTTTET | LLLDDDDDDDDDDLDLL | 2SSSSSSSSSSSSSS2 | 36 | 6952-6968 |
| 6 | GEATTATAGGTTTTTET | LLLDDDDODDDDDLDLL | 2SSSSSSSSSSSSSS2 | 36 | 6952-6968 |
| 6 | GEATTATAGGTTTTTET | LLLDDDDDODDDDLDLL | 2SSSSSSSSSSSSSS2 | 36 | 6952-6968 |
| 6 | GEATTATAGGTTTTTET | LLODDDDDDDDDDLLLL | 2SSSSSSSSSSSSSS2 | 36 | 6952-6968 |
| 6 | GEATTATAGGTTTTTET | LLDDDDDODDDDDLLLL | 2SSSSSSSSSSSSSS2 | 36 | 6952-6968 |
| 6 | GEATTATAGGTTTTTET | LLDDDDDDODDDDLLLL | 2SSSSSSSSSSSSSS2 | 36 | 6952-6968 |
| 6 | GEATTATAGGTTTTTET | LLDDDDDDDODDDLLLL | 2SSSSSSSSSSSSSS2 | 36 | 6952-6968 |
| 6 | GEATTATAGGTTTTTET | MLDDDDDDDDDDDLLLM | S2SSSSSSSSSSSSS2 | 36 | 6952-6968 |
| 6 | GEATTATAGGTTTTTET | MLDDDDDDDDDDDLLMM | S2SSSSSSSSSSSS2S | 36 | 6952-6968 |
| 6 | GEATTATAGGTTTTTET | MLDDDDDDDDDDDLLLM | S2SSSSSSSSSSSS22 | 36 | 6952-6968 |
| 6 | GEATTATAGGTTTTTET | MLDDDDDDDDDDDLLMM | S2SSSSSSSSSSS22S | 36 | 6952-6968 |
| 21 | GEATTATAGGTTTTUCTE | LLDDDDDDDDDLDDODLL | 2SSSSSSSSSSSSSSS2 | 41 | 6951-6968 |
| 22 | GEATTATAGGTTTUTCTE | LLDDDDDDDDDLDODDLL | 2SSSSSSSSSSSSSSS2 | 41 | 6951-6968 |
| 23 | GEATTATAGGTTUTTCTE | LLDDDDDDDDDLODDDLL | 2SSSSSSSSSSSSSSS2 | 41 | 6951-6968 |
| 24 | GEATTATAGGTTUTTET | LLDDDDDDDDDDOLLLL | 2SSSSSSSSSSSSSS2 | 36 | 6952-6968 |
| 25 | GEATTATAGGTUTTTET | LLLDDDDDDDDODLDLL | 2SSSSSSSSSSSSSS2 | 36 | 6952-6968 |
| 25 | GEATTATAGGTUTTTET | LLDDDDDDDDDODLLLL | 2SSSSSSSSSSSSSS2 | 36 | 6952-6968 |
| 26 | GEATTATAGGUTTTTCTE | LLDDDDDDDDOLDDDDLL | 2SSSSSSSSSSSSSSS2 | 41 | 6951-6968 |
| 27 | GEATTATAGGUTTTTET | LLLDDDDDDDODDLDLL | 2SSSSSSSSSSSSSS2 | 36 | 6952-6968 |
| 27 | GEATTATAGGUTTTTET | LLDDDDDDDDODDLLLL | 2SSSSSSSSSSSSSS2 | 36 | 6952-6968 |
| 28 | GEATTAUAGGTTTTTET | LLDDDDODDDDDDLLLL | 2SSSSSSSSSSSSSS2 | 36 | 6952-6968 |
| 29 | GEATUATAGGTTTTTET | LLDDODDDDDDDDLLLL | 2SSSSSSSSSSSSSS2 | 36 | 6952-6968 |
| 30 | GEAUTATAGGTTTTTET | LLDODDDDDDDDDLLLL | 2SSSSSSSSSSSSSS2 | 36 | 6952-6968 |
| 11 | GTTTTAAGATTGTGGTGTTT | LLLDDDDODDDDDDDDDLLL | SsssssssssssssSSSSS | 39 | 26358-26377 |
| 11 | GTTTTAAGATTGTGGTGTTT | LLLDDDDODDDDDDDDDLLL | SSSSSSSSSSSSSSSSSS2 | 39 | 26358-26377 |

TABLE 4
Compound (CMP) structures

| SEQ | CMP | |
|---|---|---|
| ID | ID | |
| NO | NO | HELM string (5′ to 3′) |
| 1 | 1 1 | RNA1{[LR](A)[sP].[LR](G)[sP].[dR](G)[sP].[dR](C)[sP].[LR](A)[sP].[dR](T)[sP].[dR](T)[sP].[dR](A)[sP].[dR](T)[sP].[dR](A)[sP].[dR]( |
| | | G)[sP].[dR](G)[sP].[dR](T)[sP][dR](T)[sP].[dR](T)[sP].[LR](T)[sP].[LR](T)[sP].[LR][(5meC])}$$$$V2.0 |
| 2 | 2_1 | RNA1{[LR](A)[PS2].[LR](A)[sP].[dR](A)[sP].[mR](A)[sP].[LR](G)[sP].[dR](T)[sP].[dR](T)[sP].[dR](T)[sP].[dR](C)[sP].[dR](T)[sP].[dR |
| | | ](C)[sP].[dR](A)[sP].[dR](G)[sP].[dR](A)[sP].[dR](T)[sP].[dR](A)[sP].[mR](G)[sP].[LR](G)[PS2].[LR]([5meC])}$$$$V2.0 |
| 3 | 3_1 | RNA1{[LR](G)[sP].[LR](G)[sP].[mR](A)[sP].[dR](C)[sP].[dR](A)[sP].[dR](T)[sP].[LR](G)[sP].[dR](T)[sP].[dR](T)[sP].[dR](A)[sP].[dR]( |
| | | A)[sP][sP].[dR](T)[sP].[dR](T)[sP].[dR](T)[sP].[dR](T)[sP].[dR](A)[sP].[dR](C)[sP].[LR](T)[PS2].[LR](T)####V2.0 |
| 4 | 4_1 | RNA1{[LR](T)[PS2].[LR](G)[sP].[LR](A)[sP].[dR](A)[sP].[dR](C)[sP].[dR](T)[sP].[dR](T)[sP].[dR](G)[sP].[dR](T)[sP].[dR](T)[sP].[dR] |
| | | (A)[sP].[dR](T)[sP].[dR](A)[sP].[dR](T)[sP].[dR](A)[sP].[dR](C)[sP].[LR]([5meC])[PS2].[LR](T)[PS2].[LR](G)}$$$$V2.0 |
| 4 | 4_2 | RNA1{[LR](T)[PS2].[LR](G)[sP].[LR](A)[sP].[mR](A)[sP].[dR](C)[sP].[dR](T)[sP].[dR](T)[sP].[dR](G)[P].[dR](T)[sP].[dR](T)[sP].[dR |
| | | ](A)[sP].[dR](T)[sP]0[dR](A)[sP].[dR](T)[sP].[dR](A)[sP].[dR](C)[sP].[LR]([5meC])[sP].[LR](T)[PS2].(LR)(G)$$$$V2.0 |
| 5 | 5_1 | RNA1{[LR](T)[PS2].[LR](A)[PS2].[dR](G)[sP].[dR](A)[sP].[LR](G)[sP].[dR](T)[sP].[dR](T)[sP].[dR](T)[sP].[dR](T)[sP].[dR](G)[sP].[d |
| | | R](T)[sP].[dR](G)[sP].[dR](T)[sP].[dR](G)[sP].[dR](T)[sP].[MOE]([5meC])[sP].[MOE](T)}$$$$V2.0 |
| 5 | 5_2 | RNA1{[LR](T)[PS2].[LR](A)[sP].[dR](G)[sP].[dR](A)[sP].[LR](G)[sP].[dR](T)[sP].[dR](T)[sP].[dR](T)[sP].[dR](T)[sP].[dR](G)[P].[dR] |
| | | (T)[sP].[dR](G)[sP].[dR](T)[sP].[dR](G)[sP].[dR](T)[sP].[MOE]([5meC])[sP].[MOE](T)}$$$$V2.0 |
| 6 | 6_1 | RNA1{[LR](G)[sP].[LR]([5meC])[sP].[dR](A)[P].[dR](T)[sP].[dR](T)[sP].[dR](A)[sP].[dR](T)[sP].[dR](A)[sP].[dR](G)[sP].[dR](G)[sP]. |
| | | [dR](T)[sP].[dR](T)[sP].[dR](T)[sP].[LR](T)[sP].[LR](T)[sP].[LR]([5meC])[sP].[LR](T)}$$$$V2.0 |
| 7 | 7_1 | RNA1{[MOE](G)[sP].[LR](G)[sP].[dR](C)[sP].[LR](A)[sP].[dR](T)[sP].[dR](T)[sP].[dR](A)[sP].[dR](T)[sP].[dR](A)[sP].[dR](G)[sP].[d |
| | | R](G)[sP].[dR](T)[sP].[dR](T)[sP].[LR](T)[sP].[MOE](T)[sP].[MOE](T)[sP].[MOE]([5meC])}$$$$V2.0 |
| 7 | 7_2 | RNA1{[LR](G)[sP].[LR](G)[sP].[dR](C)[sP].[LR](A)[sP].[dR](T)[sP].[dR](T)[sP].[dR](A)[sP].[dR](T)[sP].[mR](A)[sP].[dR](G)[sP].[dR]( |
| | | G)[sP].[dR](T)[sP].[dR](T)[sP].[dR](T)[sP].[LR](T)[sP].[LR](T)[P].[LR]([5meC])}$$$$V2.0 |
| 8 | 8_1 | RNA1{[MOE](G)[sP].[MOE](G)[sP].[MOE]([5meC])[sP].[LR](A)[PS2].[dR](T)[sP].[dR](T)[sP].[dR](A)[sP].[dR](T)[sP].[mR](A)[sP].[d |
| | | R](G)[sP].[dR](G)[sP].[dR](T)[sP].[dR](T)[sP].[LR](T)[PS2].[LR](T)[PS2].[LR](T)[PS2].[MOE]([5meC])}$$$$V2.0 |
| 2 | 2_2 | RNA1{[LR](A)[PS2].[LR](A)[PS2].[dR](A)[sP].[mR](A)[sP].[LR](G)[sP].[dR](T)[sP].[dR](T)[sP].[dR](T)[sP].[dR](C)[sP].[dR](T)[sP].[d |
| | | R](C)[sP].[dR](A)[sP].[dR](G)[sP].[dR](A)[sP].[dR](T)[sP].[dR](A)[sP].[dR](G)[sP].[LR](G)[PS2].[LR]([5meC])}$$$$V2.0 |
| 9 | 9_1 | RNA1{[LR](T)[PS2].[LR](G)[sP].[dR](G)[sP].[LR](A)[sP].[dR](C)[sP].[dR](A)[sP].[mR](U)[sP].[dR](G)[sP].[dR](T)[sP].[LR](T)[sP].[d |
| | | R](A)[sP].[dR](A)[sP].[dR](T)[sP].[dR](T)[sP].[dR](T)[sP].[dR](T)[sP].[LR](A)[PS2].[LR]([5meC])}$$$$V2.0 |
| 10 | 10_1 | RNA1{[LR](G)[PS2].[LR](G)[PS2].[dR](A)[sP].[dR](C)[sP].[dR](A)[sP].[dR](T)[sP].[LR](G)[sP].[mR](U)[sP].[dR](T)[sP].[mR](A)[sP].[ |
| | | dR](A)[sP].[dR](T)[sP].[dR](T)[sP].[dR](T)[sP].[dR](T)[sP].[dR](A)[sP].[dR](C)[sP].[LR](T)[PS2].[LR](T)}$$$$V2.0 |
| 11 | 11_1 | RNA1{[LR](G)[sP].[LR](T)[sP].[LR](T)[sP].[dR](T)[sP].[dR](T)[sP].[mR](A)[sP].[dR](A)[sP].[dR](G)[sP].[dR](A)[sP].[dR](T)[sP].[dR]( |
| | | T)[sP].[dR](G)[sP].[dR](T)[sP].[dR](G)[sP].[dR](G)[sP].[dR](T)[sP].[dR](G)[sP].[LR](T)[sP].[LR](T)[PS2].[LR](T)}$$$$V2.0 |
| 4 | 4_3 | RNA1{[LR](T)[PS2].[LR](G)[sP].[LR](A)[sP].[dR](A)[sP].[dR](C)[sP].[dR](T)[sP].[dR](T)[sP].[dR](G)[sP].[dR](T)[sP].[dR](T)[sP].[dR] |
| | | (A)[sP].[dR](T)[sP].[dR](A)[sP].[dR](T)[sP].[dR](A)[sP].[dR](C)[sP].[LR]([5meC])[sP].[LR](T)[PS2].[LR](G)}$$$$V2.0 |
| 12 | 12_1 | RNA1{[LR](T)[PS2].[LR](A)[PS2].[dR](G)[sP].[dR](A)[sP].[LR](G)[sP].[dR](T)[sP].[dR](T)[sP].[dR](T)[sP].[mR](U)[sP].[dR](G)[sP].[d |
| | | R](T)[sP].[dR](G)[sP].[dR](T)[sP].[dR](G)[sP].[dR](T)[sP].[MOE]([5meC])[sP].[MOe](T)}$$$$V2.0 |
| 5 | 5_3 | RNA1{[MOE](T)[sP].[MOE](A)[sP].[MOE](G)[sP].[MOE](A)[sP].[LR](G)[PS2].[dR](T)[sP].[dR](T)[sP].[dR](T)[sP].[dR](T)[sP].[dR](G |
| | | )[sP].[dR](T)[sP].[dR](G)[sP].[dR](T)[sP].[dR](G)[sP].[dR](T)[sP].[LR]([5meC])[PS2].[MOE](T)}$$$$V2.0 |
| 13 | 13_1 | RNA1{[MOE](T)[sP].[MOE](A)[sP].[MOE](G)[sP].[MOE](A)[sP].[LR](G)[PS2].[dR](T)[sP].[dR](T)[sP].[dR](T)[sP].[dR](T)[sP].[dR](G |
| | | )[sP].[dR](T)[sP].[dR](G)[sP].[mR](U)[sP].[dR](G)[sP].[dR](T)[sP].[LR]([5meC])[PS2).[MOE](T)}$$$$V2.0 |
| 14 | 14_1 | RNA1{[LR](A)[PS2].[LR](T)[sP].[LR](A)[sP].[LR](G)[sP].[dR](A)[sP].[dR]([PPG])[P].[dR](T)[sP].[dR](T)[sP].[dR](T)[sP].[dR](T)[sP]. |
| | | [dR](G)[sP].[dR](T)[sP].[dR](G)[sP].[dR](T)[sP].[dR](G)[sP].[LR](T)[PS2].[LR]([5meC])}$$$$V2.0 |
| 15 | 15_1 | RNA1{[LR](A)[PS2].[LR](A)[sP].[LR](A)[sP].[dR](A)[sP].[dR]([PPG])[sP].[dR](T)[sP].[dR](T)[sP].[dR](T)[sP].[dR](C)[sP].[dR](T)[sP]. |
| | | [dR](C)[sP].[dR](A)[sP].[dR](G)[sP].[dR](A)[sP].[dR](T)[sP].[dR](A)[sP].[LR](G)[sP].[LR](G)[PS2].[LR]([5meC])}$$$$V2.0 |
| 16 | 16_1 | RNA1{[LR](A)[PS2].[LR](A)[sP].[LR](A)[sP].[dR](A)[sP].[dR](G)[sP].[dR](T)[sP].[dR](T)[sP].[dR](T)[sP].[dR](C)[sP].[dR](T)[sP].[dR] |
| | | (C)[sP].[dR](A)[sP].[dR]([PPG])[sP].[dR](A)[sP].[dR](T)[sP].[dR](A)[sP].[LR](G)[sP].[LR](G)[PS2].[LR]([5meC])}$$$$V2.0 |
| 16 | 16_2 | RNA1{[MOE](A)[P].[LR](A)[PS2].[LR](A)[sP].[dR](A)[sP].[dR](G)[sP].[dR](T)[sP].[dR](T)[sP].[dR](T)[sP].[dR](C)[sP].[dR](T)[sP].[d |
| | | R](C)[sP].[dR](A)[sP].[dR]([PPG])[sP].[dR](A)[sP].[dR](T)[sP].[dR](A)[sP].[LR](G)[sP].[LR](G)[PS2].[MOE]([5meC])}$$$$V2.0 |
| 16 | 16_3 | RNA1{[MOE](A)[sP].[LR](A)[PS2].[LR](A)[sP].[dR](A)[sP].[dR](G)[sP].[dR](T)[sP].[dR](T)[sP].[dR](T)[sP].[dR](C)[sP].[dR](T)[sP].[d |
| | | R](C)[sP].[dR](A)[sP].[dR]([PPG])[sP].[dR](A)[sP].[dR](T)[sP].[dR](A)[sP].[LR](G)[PS2].[LR](G)[PS2].[MOE]([5meC])}$$$$V2.0 |
| 2 | 2_3 | RNA1{[LR](A)[PS2].[LR](A)[sP].[dR](A)[sP].[mR](A)[sP].[LR](G)[sP].[dR](T)[sP].[dR](T)[sP].[dR](T)[sP].[dR](C)[sP].[dR](T)[sP].[dR |
| | | ](C)[sP].[dR](A)[sP].[dR](G)[sP].[dR](A)[sP].[dR](T)[sP].]dR](A)[sP].[dR](G)[sP].[LR](G)[PS2].[LR]([5meC])}$$$$V2.0 |
| 2 | 2_4 | RNA1{[LR](A)[PS2].[LR](A)[PS2].[dR](A)[sP].[mR](A)[sP].[LR](G)[P].[dR](T)[sP].[dR](T)[sP].[dR](T)[sP].[dR](C)[sP].[dR](T)[sP].[d |
| | | R](C)[sP].[dR](A)[sP].[dR](G)[sP].[dR](A)[sP].[dR](T)[sP].[dR](A)[sP].[mR](G)[sP].[LR](G)[PS2].[LR]([5meC])}$$$$V2.0 |
| 2 | 2_5 | RNA1{[LR](A)[PS2].[LR](A)[sP].[LR](A)[sP].[dR](A)[sP].[dR](G)[sP].[dR](T)[sP].[dR](T)[sP].[dR](T)[sP].[dR](C)[sP].[dR](T)[sP].[dR] |
| | | (C)[sP].[mR](A)[sP].[dR](G)[sP].[dR](A)[sP].[dR](T)[sP].[dR](A)[sP].[LR](G)[sP].[LR](G)[PS2].[LR]([5meC])}$$$$V2.0 |
| 2 | 2_6 | RNA1{[LR](A)[PS2].[MOE](A)[sP].[MOE](A)[sP].[dR](A)[sP].[dR](G)[sP].[dR](T)[sP].[dR](T)[sP].[dR](T)[sP].[dR](C)[sP].[dR](T)[sP]. |
| | | [dR](C)[sP].[mR](A)[sP].[dR](G)[sP].[dR](A)[sP].[dR](T)[sP].[dR](A)[sP].[LR](G)[PS2].[LR](G)[PS2].[MOE]([5meC])}$$$$V2.0 |
| 2 | 2_7 | RNA1{[LR](A)[PS2].[LR](A)[sP].[LR](A)[sP].[dR](A)[sP].[dR](G)[sP].[dR](T)[sP].[dR](T)[sP].[dR](T)[sP].[dR](C)[sP].[dR](T)[sP].[mR |
| | | ](C)[sP].[dR](A)[sP].[dR](G)[sP].[dR](A)[sP].[dR](T)[sP].[dR](A)[sP].[LR](G)[sP].[LR](G)[PS2].[LR]([5me])}$$$$V2.0 |
| 2 | 2_8 | RNA1{[MOE](A)[sP].[MOE](A)[sP].[LR](A)[PS2].[dR](A)[sP].[dR](G)[sP].[dR](T)[sP].[dR](T)[sP].[dR](T)[sP].[dR](C)[sP].[dR](T)[sP]. |
| | | [mR](C)[sP].[dR](A)[sP].[dR](G)[sP].[dR](A)[sP].[dR](T)[sP].[dR](A)[sP].[LR](G)[sP].[LR](G)[PS2].[MOE]([5meC])}$$$$v2.0 |
| 2 | 2_9 | RNA1{[MOE](A)[sP].[LR](A)[sP].[LR](A)[sP].[dR](A)[sP].[dR](G)[sP].[dR](T)[sP].[dR](T)[sP].[dR](T)[sP].[dR](C)[sP].[dR](T)[sP].[dR |
| | | ](C)[sP].[mR](A)[sP].[dR](G)[sP].[dR](A)[sP].[dR](T)[sP].[dR](A)[sP].[LR](G)[sP].[LR](G)[sP].[MOE]([5meC])}$$$$V2.0 |
| 2 | 2_10 | RNA1{[LR](A)[PS2].[LR](A)[sP].[LR](A)[sP].[dR](A)[sP].[dR](G)[sP].[dR](T)[sP].[dR](T)[sP].[dR](T)[sP].[mR](C)[sP].[dR](T)[sP].[dR |
| | | ](C)[sP].[dR](A)[sP].[dR](G)[sP].[dR](A)[sP].[dR](T)[sP].[dR](A)[sP].[LR](G)[sP].[LR](G)[PS2].[LR]([5meC])}$$$$V2.0 |
| 2 | 2_11 | RNA1{[MOE](A)[sP].[LR](A)[sP].[LR](A)[sP].[dR](A)[sP].[dR](G)[sP].[dR](T)[sP].[dR](T)[sP].[dR](T)[sP].[mR](C)[P].[dR](T)[sP].[d |
| | | R](C)[sP].[dR](A)[sP].[dR](G)[sP].[dR](A)[sP].[dR](T)[sP].[dR](A)[sP].[LR](G)[sP].[LR](G)[PS2].[MOE]([5meC])}$$$$V2.0 |
| 2 | 2_12 | RNA1{[MOE](A)[sP].[LR](A)[PS2].[LR](A)[sP].[dR](A)[sP].[dR](G)[P].[dR](T)[sP].[dR](T)[sP].[dR](T)[sP].[dR](C)[sP].[dR](T)[sP].[d |
| | | R](C)[sP].[dR](A)[sP].[dR](G)[sP].[dR](A)[sP].[dR](T)[sP].[mR](A)[sP].[LR](G)[sP].[LR](G)[PS2].[MOE]([5meC])}$$$$V2.0 |
| 17 | 17_1 | RNA1{[MOE](A)[sP].[MOE](A)[sP].[LR](A)[sP].[dR](A)[sP].[dR](G)[sP].[dR](T)[sP].[dR](T)[sP].[mR](U)[sP].[dR](C)[sP].[dR](T)[sP].[ |
| | | dR](C)[sP].[dR](A)[sP].[dR](G)[sP].[dR](A)[sP].[dR](T)[sP].[dR](A)[sP].[LR](G)[sP].[LR](G)[PS2].[MOE]([5meC])}$$$$V2.0 |
| 18 | 18_1 | RNA1{[LR](A)[PS2].[LR](G)[sP].[dR](G)[sP].[dR](C)[sP].[LR](A)[sP].[dR](T)[sP].[dR](T)[sP].[dR](A)[sP].[dR](T)[sP].[dR](A)[sP].[dR] |
| | | (G)[sP].[dR](G)[sP].[mR](U)[sP].[dR](T)[sP].[dR](T)[sP].[LR](T)[sP].[LR](T)[PS2].[LR]([5meC])}$$$$V2.0 |
| 19 | 19_1 | RNA1{[LR](A)[PS2].[LR](G)[sP].[dR](G)[sP].[dR](C)[sP].[LR](A)[sP].[mR](U)[sP].[dR](T)[sP].[dR](A)[sP].[dR](T)[sP].[dR](A)[sP].[d |
| | | R](G)[sP].[dR](G)[sP].[dR](T)[sP].[dR](T)[sP].[dR](T)[sP].[LR](T)[sP].[LR](T)[PS2].[LR]([5meC])}$$$$V2.0 |
| 20 | 20_1 | RNA1{[LR](G)[PS2].[LR]([5meC])[sP].[dR](A)[sP].[dR](T)[sP].[dR](T)[sP].[dR](A)[sP].[dR](T)[sP].[dR](A)[sP].[dR](G)[sP].[dR](G)[s |
| | | P].[dR](T)[sP].[LR](T)[sP].[dR](T)[sP].[dR](T)[sP].[dR](T)[sP].[dR](C)[sP].[LR][PS2].[LR]([5meC])}$$$$V2.0 |
| 20 | 20_2 | RNA1{[LR](G)[PS2].[LR]([5meC])[sP].[mR](A)[sP].[dR](T)[sP].[dR](T)[sP].[dR](A)[sP].[dR](T)[sP].[dR](A)[sP].[dR](G)[sP].[dR](G)[s |
| | | P].[dR](T)[sP].[LR](T)[sP].[dR](T)[sP].[dR](T)[sP].[dR](T)[sP].[dR](C)[sP].[LR][PS2].[LR]([5meC])}$$$$V2.0 |
| 20 | 20_3 | RNA1{[LR](G)[PS2].[LR]([5meC])[sP].[dR](A)[sP].[dR](T)[sP].[dR](T)[sP].[dR](A)[sP].[dR](T)[sP].[mR](A)[sP].[dR](G)[sP].[dR](G)[s |
| | | P].[dR](T)[sP].[LR](T)[sP].[dR](T)[sP].[dR](T)[sP].[dR](T)[sP].[dR](C)[sP].[LR][PS2].[LR]([5meC])}$$$$V2.0 |
| 20 | 20_4 | RNA1{[LR](G)[PS2].[LR]([5meC])[sP].[dR](A)[sP].[dR](T)[sP].[dR](T)[sP].[dR](A)[sP].[dR](T)[sP].[dR](A)[sP].[mR](G)[P].[dR](G)[s |
| | | P].[dR](T)[sP].[LR](T)[sP].[dR](T)[sP].[dR](T)[sP].[dR](T)[sP].[dR](C)[sP].[LR](T)[PS2].[LR]([5meC])}$$$$V2.0 |
| 20 | 20_5 | RNA1{[LR](G)[PS2].[LR]([5meC])[sP].[dR](A)[P].[dR](T)[sP].[dR](T)[sP].[dR](A)[sP].[dR](T)[sP].[dR](A)[sP].[dR](G)[P].[dR](G)[s |
| | | P].[dR](T)[sP].[LR](T)[sP].[dR](T)[sP].[dR](T)[sP].[dR](T)[sP].[dR](C)[sP].[LR](T)[PS2].[MOE]([5meC])}$$$$V2.0 |
| 20 | 20_6 | RNA1{[LR](G)[PS2].[LR]([5meC])[sP].[dR](A)[sP].[dR](T)[sP].[dR](T)[sP].[dR](A)[sP].[dR](T)[sP].[dR](A)[sP].[dR](G)[sP].[mR](G)[s |
| | | P].[dR](T)[sP].[LR](T)[sP].[dR](T)[sP].[dR](T)[sP].[dR](T)[sP].[dR](C)[P].[LR](T)[PS2].[LR]([5meC])}$$$$V2.0 |
| 6 | 6_2 | RNA1{[LR](G)[PS2].[LR]([5meC])[sP].[LR](A)[P].[dR](T)[sP].[dR](T)[sP].[dR](A)[sP].[dR](T)[sP].[dR](A)[P].[dR](G)[sP].[dR](G)[s |
| | | P].[dR](T)[sP].[LR](T)[sP].[dR](T)[sP].[dR](T)[sP].[dR](T)[sP].[LR]([5meC])[PS2].[LR](T)}$$$$V2.0 |
| 6 | 6_3 | RNA1{[LR](G)[PS2].[LR]([5meC])[P].[LR](A)[sP].[dR](T)[sP].[dR](T)[sP].[dR](A)[sP].[dR](T)[sP].[mR](A)[sP].[dR](G)[sP].[dR](G)[s |
| | | P].[dR](T)[sP].[LR](T)[sP].[dR](T)[sP].[dR](T)[sP].[dR](T)[sP].[LR]([5meC])[PS2].[LR](T)}$$$$V2.0 |
| 6 | 6_4 | RNA1{[LR](G)[PS2].[LR]([5meC])[sP].[LR](A)[sP].[dR](T)[sP].[dR](T)[P].[dR](A)[sP].[dR](T)[sP].[dR](A)[sP].[mR](G)[sP].[dR](G)[s |
| | | P].[dR](T)[sP].[LR](T)[sP].[dR](T)[sP].[dR](T)[sP].[dR](T)[sP].[LR]([5meC])[PS2].[LR](T)}$$$$V2.0 |
| 6 | 6_5 | RNA1{[LR](G)[PS2].[LR]([5meC])[sP].[mR](A)[sP].[dR](T)[sP].[dR](T)[sP].[dR](A)[sP].[dR](T)[sP].[dR](A)[sP].[dR](G)[sP].[dR](G)[s |
| | | P].[dR](T)[sP].[LR](T)[sP].[dR](T)[sP].[dR](T)[sP].[LR](T)[sP].[LR]([5meC])[PS2].[LR](T)}$$$$V2.0 |
| 6 | 6_6 | RNA1{[LR](G)[PS2].[LR]([5meC])[sP].[dR](A)[sP].[dR](T)[sP].[dR](T)[P].[dR](A)[sP].[dR](T)[sP].[mR](A)[sP].[dR](G)[sP].[dR](G)[s |
| | | P].[dR](T)[sP].[dR](T)[sP].[dR](T)[sP].[LR](T)[sP].[LR](T)[sP].[LR]([5meC])[PS2].[LR](T)}$$$$V2.0 |
| 6 | 6_7 | RNA1{[LR](G)[PS2].[LR]([5meC])[sP].[dR](A)[sP].[dR](T)[sP].[dR](T)[sP].[dR](A)[sP].[dR](T)[sP].[dR](A)[P].[mR](G)[sP].[dR](G)[s |
| | | P].[dR](T)[sP].[dR](T)[sP].[dR](T)[sP].[LR](T)[sP].[LR](T)[sP].[LR]([5meC])[PS2].[LR](T)}$$$$V2.0 |
| 6 | 6_8 | RNA1{[LR](G)[PS2].[LR]([5meC])[sP].[dR](A)[P].[dR](T)[sP].[dR](T)[sP].[dR](A)[sP].[dR](T)[sP].[dR](A)[sP].[dR](G)[sP].[mR](G)[s |
| | | P].[dR](T)[sP].[dR](T)[sP].[dR](T)[sP].[LR](T)[sP].[LR](T)[sP].[LR]([5meC])[PS2].[LR](T)}$$$$V2.0 |
| 6 | 6_9 | RNA1{[MOE](G)[sP].[LR]([5meC])[PS2].[dR](A)[sP].[dR](T)[sP].[dR](T)[sP].[dR](A)[sP].[dR](T)[sP].[dR](A)[sP].[dR](G)[sP].[dR](G)[ |
| | | sP].[dR](T)[sP].[dR](T)[sP].[dR](T)[sP].[LR](T)[sP].[LR](T)[sP].[LR]([5meC])[PS2].[MOE](T)}$$$$V2.0 |
| 6 | 6_10 | RNA1{[MOE](G)[sP].[LR]([5meC])[PS2].[dR](A)[sP].[dR](T)[sP].[dR](T)[sP].[dR](A)[sP].[dR](T)[sP].[dR](A)[sP].[dR](G)[sP].[dR](G)[ |
| | | sP].[dR](T)[sP].[dR](T)[sP].[dR](T)[sP].[LR](T)[sP].[LR](T)[PS2].[MOE]([5meC])[sP].[MOE](T)}$$$$V2.0 |
| 6 | 6_11 | RNA1{[MOE](G)[sP].[LR]([5meC])[PS2].[dR](A)[sP].[dR](T)[sP].[dR](T)[sP].[dR](A)[sP].[dR](T)[sP].[dR](A)[sP].[dR](G)[sP].[dR](G)[ |
| | | sP].[dR](T)[sP].[dR](T)[sP].[dR](T)[sP].[LR](T)[sP].[LR](T)[PS2].[LR]([5meC])[PS2].[MOE](T)}$$$$V2.0 |
| 6 | 6_12 | RNA1{[MOE](G)[sP].[LR]([5meC])[PS2].[dR](A)[sP].[dR](T)[sP].[dR](T)[sP].[dR](A)[sP].[dR](T)[sP].[dR](A)[sP].[dR](G)[sP].[dR](G)[ |
| | | sP].[dR](T)[sP].[dR](T)[sP].[dR](T)[sP].[LR](T)[PS2].[LR](T)[PS2].[MOE]([5meC])[sP].[MOE](T)}$$$$V2.0 |
| 21 | 21_1 | RNA1{[LR](G)[PS2].[LR]([5meC])[sP].[dR](A)[sP].[dR](T)[sP].[dR](T)[sP].[dR](A)[sP].[dR](T)[sP].[dR](A)[sP].[dR](G)[sP].[dR](G)[s |
| | | P].[dR](T)[sP].[LR](T)[sP].[dR](T)[sP].[dR](T)[sP].[mR](U)[sP].[dR](C)[sP].[LR](T)[PS2].[LR]([5meC])}$$$$V2.0 |
| 22 | 22_1 | RNA1{[LR](G)[PS2].[LR]([5meC])[sP].[dR](A)[sP].[dR](T)[sP].[dR](T)[sP].[dR](A)[sP].[dR](T)[sP].[dR](A)[sP].[dR](G)[sP].[dR](G)[s |
| | | P].[dR](T)[sP].[LR](T)[sP].[dR](T)[sP].[mR](U)[sP].[dR](T)[sP].[dR](C)[sP].[LR](T)[PS2].[LR]([5meC])}$$$$V2.0 |
| 23 | 23_1 | RNA1{[LR](G)[PS2].[LR]([5meC])[sP].[dR](A)[sP].[dR](T)[sP].[dR](T)[sP].[dR](A)[sP].[dR](T)[sP].[dR](A)[sP].[dR](G)[sP].[dR](G)[s |
| | | P].[dR](T)[sP].[LR](T)[sP].[mR](U)[sP].[dR](T)[sP].[dR](T)[sP].[dR](C)[sP].[LR](T)[PS2].[LR]([5meC])}$$$$V2.0 |
| 24 | 24_1 | RNA1{[LR](G)[PS2].[LR]([5meC])[sP].[dR](A)[sP].[dR](T)[sP].[dR](T)[sP].[dR](A)[sP].[dR](T)[sP].[dR](A)[sP].[dR](G)[sP].[dR](G)[s |
| | | P].[dR](T)[sP].[dR](T)[sP].[mR](U)[sP].[LR](T)[sP].[LR](T)[sP].[LR]([5meC])[PS2].[LR](T)}$$$$V2.0 |
| 25 | 25_1 | RNA1{[LR](G)[PS2].[LR]([5meC])[sP].[LR](A)[sP].[dR](T)[sP].[dR](T)[sP].[dR](A)[sP].[dR](T)[P].[dR](A)[P].[dR](G)[sP].[dR](G)[s |
| | | P].[dR](T)[sP].[mR](U)[sP].[dR](T)[sP].[LR](T)[sP].[dR](T)[sP].[LR]([5meC])[PS2].[LR](T)}$$$$V2.0 |
| 25 | 25_2 | RNA1{[LR](G)[PS2].[LR]([5meC])[sP].[dR](A)[sP].[dR](T)[sP].[dR](T)[sP].[dR](A)[P].[dR](T)[sP].[dR](A)[sP].[dR](G)[sP].[dR](G)[s |
| | | P].[dR](T)[sP].[mR](U)[sP].[dR](T)[sP].[LR](T)[sP].[LR](T)[sP].[LR]([5meC])[PS2].[LR](T)}$$$$V2.0 |
| 26 | 26_1 | RNA1{[LR](G)[PS2].[LR]([5meC])[sP].[dR](A)[sP].[dR](T)[sP].[dR](T)[sP].[dR](A)[sP].[dR](T)[sP].[dR](A)[sP].[dR](G)[sP].[dR](G)[s |
| | | P].[mR](U)[sP].[LR](T)[sP].[dR](T)[sP].[dR](T)[sP].[dR](T)[sP].[dR](C)[sP].[LR](T)[PS2].[LR]([5meC])}$$$$V2.0 |
| 27 | 27_1 | RNA1{[LR](G)[PS2].[LR]([5meC])[sP].[LR](A)[sP].[dR](T)[sP].[dR](T)[sP].[dR](A)[sP].[dR](T)[sP].[dR](A)[sP].[dR](G)[sP].[dR](G)[s |
| | | P].[mR](U)[sP].[dR](T)[sP].[dR](T)[sP].[LR](T)[sP].[dR](T)[sP].[LR]([5meC])[PS2].[LR](T)}$$$$V2.0 |
| 27 | 27_2 | RNA1{[LR](G)[PS2].[LR]([5meC])[sP].[dR](A)[sP].[dR](T)[sP].[dR](T)[sP].[dR](A)[sP].[dR](T)[sP].[dR](A)[sP].[dR](G)[sP].[dR](G)[s |
| | | P].[mR](U)[sP].[dR](T)[sP].[dR](T)[sP].[LR](T)[sP].[LR](T)[sP].[LR]([5meC])[PS2].[LR](T)}$$$$V2.0 |
| 28 | 28_1 | RNA1{[LR](G)[PS2].[LR]([5meC])[P].[dR](A)[sP].[dR](T)[sP].[dR](T)[sP].[dR](A)[sP].[mR](U)[sP].[dR](A)[sP].[dR](G)[sP].[dR](G)[s |
| | | P].[dR](T)[sP].[dR](T)[sP].[dR](T)[sP].[LR](T)[sP].[LR](T)[sP].[LR]([5meC])[PS2].[LR](T)}$$$$V2.0 |
| 29 | 29_1 | RNA1{[LR](G)[PS2].[LR]([5meC])[sP].[dR](A)[sP].[dR](T)[sP].[mR](U)[P].[dR](A)[sP].[dR](T)[sP].[dR](A)[sP].[dR](G)[sP].[dR](G)[s |
| | | P].[dR](T)[sP].[dR](T)[sP].[dR](T)[sP].[LR](T)[sP].[LR](T)[sP].[LR]([5meC])[PS2].[LR](T)}$$$$V2.0 |
| 30 | 30_1 | RNA1{[LR](G)[PS2].[LR]([5meC])[sP].[dR](A)[sP].[mR](U)[sP].[dR](T)[sP].[dR](A)[sP].[dR](T)[sP].[dR](A)[sP].[dR](G)[sP].[dR](G)[s |
| | | P].[dR](T)[sP].[dR](T)[sP].[dR](T)[sP].[LR](T)[sP].[LR](T)[sP].[LR]([5meC])[PS2].[LR](T)}$$$$V2.0 |
| 11 | 11_2 | RNA1{[LR](G)[sP].[LR](T)[sP].[LR](T)[P].[dR](T)[sP].[dR](T)[sP].[dR](A)[sP].[dR](A)[sP].[mR](G)[sP].[dR](A)[sP].[dR](T)[sP].[dR]( |
| | | T)[sP].[dR](G)[sP].[dR](T)[sP].[dR](G)[sP].[dR](G)[sP].[dR](T)[sP].[dR](G)[sP].[LR](T)[sP].[LR](T)[sP].[LR](T)}$$$$V2.0 |
| 11 | 11_3 | RNA1{[LR](G)[sP].[LR](T)[sP].[LR](T)[sP].[dR](T)[sP].[dR](T)[sP].[dR](A)[sP].[dR](A)[sP].[mR](G)[sP].[dR](A)[sP].[dR](T)[sP].[dR]( |
| | | T)[sP].[dR](G)[sP].[dR](T)[sP].[dR](G)[sP].[dR](G)[sP].[dR](T)[sP].[dR](G)[sP].[LR](T)[sP].[LR](T)[PS2].[LR](T)}$$$$V2.0 |

TABLE 5
Conjugate (CNJ) Structures

| SEQ | CMP | CNJ | |
|---|---|---|---|
| ID | ID | ID | |
| NO | NO | NO | HELM string (5' to 3') |
| 6 | 6_1 | 6_1 | CHEM1{[5gn2c6]}\|RNA1{P.[dR](C)P.[dR](A)P.[LR](G)[sP].[LR]([5meC])[sP].[dR](A)[sP].[dR](T)[sP].[dR](T)[sP].[dR] |
| | | | (A)[sP].[dR](T)[sP].[dR](A)[sP].[dR](G)[sP].[dR](G)[sP].[dR](T)[sP].[dR](T)[sP].[dR](T)[P].[LR](T)[sP].[LR](T)[sP].[L |
| | | | R]([5meC])[sP].[LR](T)}$CHEM1, RNA1, 1:R2-1:R1$$$V2.0 |
| 1 | 1_1 | 1_1 | CHEM1{[5gn2c6]}\|RNA1{P.[dR](C)P.[dR](A)P.[LR](A)[sP].[LR](G)[sP].[dR](G)[P].[dR](C)[sP].[LR](A)[sP].[dR](T)[s |
| | | | P].[dR](T)[sP].[dR](A)[sP].[dR](T)[sP].[dR](A)[sP].[dR](G)[sP].[dR](G)[sP].[dR](T)[sP].[dR](T)[sP].[dR](T)[sP].[LR](T) |
| | | | [sP].[LR](T)[sP].[LR]([5meC])}$CHEM1, RNA1, 1:R2-1:R1$$$V2.0 |
| 7 | 7_1 | 7_1 | CHEM1{[5gn2c6]}\|RNA1{P.[dR](C)P.[dR](A)P.[MOE](G)[sP].[LR](G)[sP].[dR](C)[sP].[LR](A)[sP].[dR](T)[sP].[dR](T)[ |
| | | | sP].[dR](A)[sP].[dR](T)[sP].[dR](A)[sP].[dR](G)[sP].[dR](G)[sP].[dR](T)[sP].[dR](T)[sP].[LR](T)[sP].[MOE](T)[sP].[M |
| | | | OE](T)[sP].[MOE]([5meC])}$CHEM1, RNA1, 1:R2-1:R1$$$V2.0 |
| 7 | 7_2 | 7_2 | CHEM1{[5gn2c6]}\|RNA1{P.[dR](C)P.[dR](A)P.[LR](G)[sP].[LR](G)[sP].[dR](C)[sP].[LR](A)[sP].[dR](T)[sP].[dR](T)[s |
| | | | P].[dR](A)[sP].[dR](T)[sP].[mR](A)[sP].[dR](G)[P].[dR](G)[sP].[dR](T)[sP].[dR](T)[sP].[dR](T)[sP].[LR](T)[sP].[LR](T |
| | | | )[sP].[LR]([5meC])}$CHEM1, RNA1, 1:R2-1:R1$$$V2.0 |
| 2 | 2_2 | 2_2 | CHEM1{[5gn2c6]}\|RNA1{P.[dR](C)P.[dR](A)P.[LR](A)[PS2].[LR](A)[PS2].[dR](A)[sP].[mR](A)[sP].[LR](G)[sP].[dR](T |
| | | | )[sP].[dR](T)[sP].[dR](T)[sP].[dR](C)[sP].[dR](T)[sP].[dR](C)[sP].[dR](A)[sP].[dR](G)[sP].[dR](A)[sP].[dR](T)[sP].[dR]( |
| | | | A)[sP].[dR](G)[sP].[LR](G)[PS2].[LR]([5meC])}$CHEM1, RNA1, 1:R2-1:R1$$$V2.0 |
| 2 | 2_1 | 2_1 | CHEM1{[5gn2c6]}\|RNA1{P.[dR](C)P.[dR](A)P.[LR](A)[PS2].[LR](A)[sP].[dR](A)[sP].[mR](A)[sP].[LR](G)[sP].[dR](T)[ |
| | | | sP].[dR](T)[P].[dR](T)[sP].[dR](C)[sP].[dR](T)[sP].[dR](C)[sP].[dR](A)[sP].[dR](G)[sP].[dR](A)[sP].[dR](T)[sP].[dR]( |
| | | | A)[sP].[mR](G)[sP].[LR](G)[PS2].[LR]([5meC])}$CHEM1, RNA1, 1:R2-1:R1$$$V2.0 |
| 3 | 3_1 | 3_1 | CHEM1{[5gn2c6]}\|RNA1{P.[dR](C)P.[dR](A)P.[LR](G)[sP].[LR](G)[sP].[mR](A)[sP].[dR](C)[sP].[dR](A)[sP].[dR](T)[s |
| | | | P].[LR](G)[sP].[dR](T)[sP].[dR](T)[sP].[dR](A)[sP].[dR](A)[sP].[dR](T)[sP].[dR](T)[sP].[dR](T)[sP].[dR](T)[sP].[dR](A)[ |
| | | | sPl.[dR](C)[sP].[LR](T)[PS2].[LR](T)}$CHEM1, RNA1, 1:R2-1:R1$$$V2.0 |
| 9 | 9_1 | 9_1 | CHEM1{[5gn2c6]}\|RNA1{P.[dR](C)P.[dR](A)P.[LR](T)[PS2].[LR](G)[P].[dR](G)[sP].[LR](A)[sP].[dR](C)[sP].[dR](A)[ |
| | | | sP].[mR](U)[sP].[dR](G)[sP].[dR](T)[sP].[LR](T)[sP].[dR](A)[sP].[dR](A)[sP].[dR](T)[sP].[dR](T)[sP].[dR](T)[sP].[dR]( |
| | | | T)[sP].[LR](A)[PS2].[LR]([5meC])}$CHEM1, RNA1, 1:R2-1:R1$$$V2.0 |
| 8 | 8_1 | 8_1 | CHEM1{[5gn2c6]}\|RNA1{P.[dR](C)P.[dR](A)P.[MOE](G)[sP].[MOE](G)[sP].[MOE]([5meC])[sP].[LR](A)[PS2].[dR](T) |
| | | | [sP].[dR](T)[sP].[dR](A)[P].[dR](T)[sP].[mR](A)[sP].[dR](G)[sP].[dR](G)[sP].[dR](T)[sP].[dR](T)[sP].[LR](T)[PS2].[LR |
| | | | [(T)[PS2].[LR](T)[PS2].[MOE]([5meC])}$CHEM1, RNA1, 1:R2-1:R1$$$V2.0 |
| 4 | 4_1 | 4_1 | CHEM1{[5gn2c6]}\|RNA1{P.[dR](C)P.[dR](A)P.[LR](T)[PS2].[LR](G)[sP].[LR](A)[sP].[dR](A)[sP].[dR](C)[sP].[dR](T)[ |
| | | | sP].[dR](T)[sP].[dR](G)[sP].[dR](T)[sP].[dR](T)[sP].[dR](A)[sP].[dR](T)[sP].[dR](A)[sP].[dR](T)[sP].[dR](A)[sP].[dR](C |
| | | | )[sP].[LR]([5meC])[PS2].[LR](T)[PS2].[LR](G)}SCHEM1, RNA1, 1:R2-1:R1$$$V2.0 |
| 4 | 4_2 | 4_2 | CHEM1{[5gn2c6]}\|RNA1{P.[dR](C)P.[dR](A)P.[LR](T)[PS2].[LR](G)[sP].[LR](A)[sP].[mR](A)[sP].[dR](C)[sP].[dR](T)[ |
| | | | sP].[dR](T)[sP].[dR](G)[sP].[dR](T)[sP].[dR](T)[sP].[dR](A)[sP].[dR](T)[sP].[dR](A)[sP].[dR](T)[sP].[dR](A)[sP].[dR](C |
| | | | )[sP].[LR]([5meC])[sP].[LR](T)[PS2].[LR](G)}$CHEM1, RNA1, 1:R2-1:R1$$$V2.0 |
| 12 | 12_1 | 12_1 | CHEM1{[5gn2c6]}\|RNA1{P.[dR](C)P.[dR](A)P.[LR](T)[PS2].[LR](A)[PS2].[dR](G)[sP].[dR](A)[sP].[LR](G)[sP].[dR](T |
| | | | )[sP].[dR](T)[sP].[dR](T)[sP].[mR](U)[P].[dR](G)[sP].[dR](T)[sP].[dR](G)[sP].[dR](T)[sP].[dR](G)[sP].[dR](T)[sP].[M |
| | | | OE]([5meC])[sP].[MOE](T)}$CHEM1, RNA1, 1:R2-1:R1$$$V2.0 |
| 5 | 5_3 | 5_3 | CHEM1{[5gn2c6]}\|RNA1{P.[dR](C)P.[dR](A)P.[MOE](T)[sP].[MOE](A)[sP].[MOE](G)[sP].[MOE](A)[sP].[LR](G)[PS2] |
| | | | .[dR](T)[sP].[dR](T)[sP].[dR](T)[P].[dR](T)[sP].[dR](G)[sP].[dR](T)[sP].[dR](G)[sP].[dR](T)[sP].[dR](G)[sP].[dR](T)[s |
| | | | P].[LR]([5meC])[PS2].[MOE](T)}$CHEM1, RNA1, 1:R2-1:R1$$$V2.0 |
| 5 | 5_1 | 5_1 | CHEM1{[5gn2c6]}\|RNA1{P.[dR](C)P.[dR](A)P.[LR](T)[PS2].[LR](A)[PS2].[dR](G)[sP].[dR](A)[sP].[LR](G)[sP].[dR](T |
| | | | )[sP].[dR](T)[sP].[dR](T)[sP].[dR](T)[P].[dR](G)[sP].[dR](T)[sP].[dR](G)[sP].[dR](T)[sP].[dR](G)[sP].[dR](T)[sP].[MO |
| | | | E]([5meC])[sP].[MOE](T)}$CHEM1, RNA1, 1:R2-1:R1$$$V2.0 |
| 13 | 13_1 | 13_1 | CHEM1{[5gn2c6]}\|RNA1{P.[dR](C)P.[dR](A)P.[MOE](T)[sP].[MOE](A)[sP].[MOE](G)[sP].[MOE](A)[sP].[LR](G)[PS2] |
| | | | .[dR](T)[sP].[dR](T)[sP].[dR](T)[sP].[dR](T)[sP].[dR](G)[sP].[dR](T)[sP].[dR](G)[sP].[mR](U)[sP].[dR](G)[sP].[dR](T)[ |
| | | | SP].[LR]([5meC])[PS2].[MOE](T)}$CHEM1, RNA1, 1:R2-1:R1$$$V2.0 |
| 14 | 14_1 | 14_1 | CHEM1{[5gn2c6]}\|RNA1{P.[dR](C)P.[dR](A)P.[LR](A)[PS2].[LR](T)[sP].[LR](A)[sP].[LR](G)[sP].[dR](A)[P].[dR]([PP |
| | | | G])[sP].[dR](T)[sP].[dR](T)[sP].[dR](T)[sP].[dR](T)[sP].[dR](G)[sP].[dR](T)[sP].[dR](G)[sP].[dR](T)[sP].[dR](G)[sP].[L |
| | | | RJ(T)[PS2].[LR]([5meC])}$CHEM1, RNA1, 1:R2-1:R1$$$V2.0 |
| 10 | 10_1 | 10_1 | CHEM1{[5gn2c6]}\|RNA1{P.[dR](C)P.[dR](A)P.[LR](G)[PS2].[LR](G)[PS2].[dR](A)[sP].[dR](C)[sP].[dR](A)[sP].[dR](T |
| | | | )[sP].[LR](G)[sP].[mR](U)[sP].[dR](T)[sP].[mR](A)[sP].[dR](A)[sP].[dR](T)[sP].[dR](T)[sP].[dR](T)[sP].[dR](T)[sP].[dR |
| | | | [(A)[sP].[dR](C)[sP].[LR](T)[PS2].[LR](T)}$CHEM1, RNA1 ,1:R2-1:R1$$$V2.0 |
| 11 | 11_1 | 11_1 | CHEM1{[5gn2c6]}\|RNA1{P.[dR](C)P.[dR](A)P.[LR](G)[sP].[LR](T)[sP].[LR](T)[sP].[dR](T)[sP].[dR](T)[sP].[mR](A)[s |
| | | | P].[dR](A)[sP].[dR](G)[sP].[dR](A)[sP].[dR](T)[sP].[dR](T)[sP].[dR](G)[sP].[dR](T)[sP].[dR](G)[sP].[dR](G)[sP].[dR](T |
| | | | )[sP].[dR](G)[sP].[LR](T)[sP].[LR](T)[PS2].[LR](T)}$CHEM1, RNA1, 1:R2-1:R1$$$V2.0 |
| 4 | 4_3 | 4_3 | CHEM1{[5gn2c6]}\|RNA1{P.[dR](C)P.[dR](A)P.[LR](T)[PS2].[LR](G)[sP].[LR](A)[sP].[dR](A)[sP].[dR](C)[sP].[dR](T)[ |
| | | | sP].[dR](T)[sP].[dR](G)[sP].[dR](T)[sP].[dR](T)[sP].[dR](A)[sP].[dR](T)[sP].[dR](A)[sP].[dR](T)[sP].[dR](A)[sP].[dR](C |
| | | | )[sP].[LR]([5meC])[sP].[LR](T)[PS2].[LR](G)}$CHEM1, RNA1, 1:R2-1:R1$$$V2.0 |
| 5 | 5_2 | 5_2 | CHEM1{[5gn2c6]}\|RNA1{P.[dR](C)P.[dR](A)P.[LR](T)[PS2].[LR](A)[sP].[dR](G)[sP].[dR](A)[sP].[LR](G)[sP].[dR](T)[ |
| | | | sP].[dR](T)[sP].[dR](T)[sP].[dR](T)[sP].[dR](G)[sP].[dR](T)[sP].[dR](G)[sP].[dR](T)[sP].[dR](G)[sP].[dR](T)[sP].[MOE |
| | | | ]([5meC])[sP].[MOE](T)}$CHEM1, RNA1, 1:R2-1:R1$$$V2.0 |
| 15 | 15_1 | 15_1 | CHEM1{[5gn2c6]}\|RNA1{P.[dR](C)P.[dR](A)P.[LR](A)[PS2].[LR](A)[sP].[LR](A)[sP].[dR](A)[sP].[dR]([PPG])[sP].[dR] |
| | | | (T)[sP].[dR](T)[sP].[dR](T)[sP].[dR](C)[sP].[dR](T)[sP].[dR](C)[sP].[dR](A)[sP].[dR](G)[sP].[dR](A)[sP].[dR](T)[sP].[d |
| | | | RI(A)[sP].[LR](G)[sP].[LR](G)[PS2].[LR]([5meC])}$CHEM1, RNA1, 1:R2-1:R1$$$V2.0 |
| 16 | 16_1 | 16_1 | CHEM1{[5gn2c6]}\|RNA1{P.[dR](C)P.[dR](A)P.[LR](A)[PS2].[LR](A)[sP].[LR](A)[sP].[dR](A)[sP].[dR](G)[sP].[dR](T)[ |
| | | | sP].[dR](T)[sP].[dR](T)[sP].[dR](C)[sP].[dR](T)[sP].[dR](C)[sP].[dR](A)[sP].[dR]([PPG])[sP].[dR](A)[sP].[dR](T)[sP].[d |
| | | | R](A)[sP].[LR](G)[sP].[LR](G)[PS2].[LR]([5meC])}$CHEM1, RNA1, 1:R2-1:R1$$$V2.0 |
| 16 | 16_2 | 16_2 | CHEM1{[5gn2c6]}\|RNA1{P.[dR](C)P.[dR](A)P.[MOE](A)[sP].[LR](A)[PS2].[LR](A)[sP].[dR](A)[sP].[dR](G)[P].[dR](T |
| | | | )[sP].[dR](T)[sP].[dR](T)[sP].[dR](C)[sP].[dR](T)[sP].[dR](C)[sP].[dR](A)[sP].[dR]([PPG])[sP].[dR](A)[sP].[dR](T)[sP].[ |
| | | | dR](A)[sP].[LR](G)[P].[LR](G)[PS2].[MOE]([5meC])}$CHEM1, RNA1, 1:R2-1:R1$$$V2.0 |
| 16 | 16_3 | 16_3 | CHEM1{[5gn2c6]}\|RNA1{P.[dR](C)P.[dR](A)P.[MOE](A)[P].[LR](A)[PS2].[LR](A)[sP].[dR](A)[sP].[dR](G)[P].[dR](T |
| | | | )[sP].[dR](T)[sP].[dR](T)[sP].[dR](C)[sP].[dR](T)[sP].[dR](C)[P].[dR](A)[sP].[dR]([PPG])[sP].[dR](A)[sP].[dR](T)[sP].[ |
| | | | dR](A)[sP].[LR](G)[PS2].[LR](G)[PS2].[MOE]([5meC])}$CHEM1, RNA1, 1:R2-1:R1$$$V2.0 |
| 2 | 2_3 | 2_3 | CHEM1{[5gn2c6]}\|RNA1{P.[dR](C)P.[dR](A)P.[LR](A)[PS2].[LR](A)[sP].[dR](A)[sP].[mR](A)[sP].[LR](G)[sP].[dR](T)[ |
| | | | sP].[dR](T)[sP].[dR](T)[sP].[dR](C)[sP].[dR](T)[sP].[dR](C)[sP].[dR](A)[sP].[dR](G)[sP].[dR](A)[sP].[dR](T)[sP].[dR]( |
| | | | A)[sP].[dR](G)[sP].[LR](G)[PS2].[LR]([5meC])}$CHEM1, RNA1, 1:R2-1:R1$$$V2.0 |
| 2 | 2_4 | 2_4 | CHEM1{[5gn2c6]}\|RNA1{P.[dR](C)P.[dR](A)P.[LR](A)[PS2].[LR](A)[PS2].[dR](A)[sP].[mR](A)[sP].[LR](G)[sP].[dR](T |
| | | | )[sP].[dR](T)[sP].[dR](T)[sP].[dR](C)[sP].[dR](T)[sP].[dR](C)[sP].[dR](A)[sP].[dR](G)[sP].[dR](A)[sP].[dR](T)[sP].[dR]( |
| | | | A)[sP].[mR](G)[sP].[LR](G)[PS2].[LR]([5meC])}$CHEM1, RNA1, 1:R2-1:R1$$$V2.0 |
| 2 | 2_5 | 2_5 | CHEM1{[5gn2c6]}\|RNA1{P.[dR](C)P.[dR](A)P.[LR](A)[PS2].[LR](A)[sP].[LR](A)[sP].[dR](A)[sP].[dR](G)[sP].[dR](T)[ |
| | | | sP].[dR](T)[sP].[dR](T)[sP].[dR](C)[sP].[dR](T)[sP].[dR](C)[sP].[mR](A)[sP].[dR](G)[sP].[dR](A)[sP].[dR](T)[sP].[dR]( |
| | | | A)[sP].[LR](G)[sP].[LR](G)[PS2].[LR]([5meC])}$CHEM1, RNA1, 1:R2-1:R1$$$V2.0 |
| 2 | 2_6 | 2_6 | CHEM1{[5gn2c6]}\|RNA1{P.[dR](C)P.[dR](A)P.[LR](A)[PS2].[MOE](A)[sP].[MOE](A)[sP].[dR](A)[sP].[dR](G)[sP].[dR] |
| | | | (T)[sP].[dR](T)[sP].[dR](T)[sP].[dR](C)[P].[dR](T)[sP].[dR](C)[sP].[mR](A)[sP].[dR](G)[sP].[dR](A)[sP].[dR](T)[sP].[d |
| | | | R](A)[sP].[LR](G)[PS2].[LR](G)[PS2].[MOE]([5meC])}$CHEM1, RNA1, 1:R2-1:R1$$$V2.0 |
| 2 | 2_7 | 2_7 | CHEM1{[5gn2c6]}\|RNA1{P.[dR](C)P.[dR](A)P.[LR](A)[PS2].[LR](A)[sP].[LR](A)[sP].[dR](A)[sP].[dR](G)[sP].[dR](T)[ |
| | | | sP].[dR](T)[sP].[dR](T)[sP].[dR](C)[sP].[dR](T)[sP].[mR](C)[sP].[dR](A)[sP].[dR](G)[sP].[dR](A)[sP].[dR](T)[sP].[dR]( |
| | | | A)[sP].[LR](G)[sP].[LR](G)[PS2].[LR]([5meC])}$CHEM1, RNA1, 1:R2-1:R1$$$V2.0 |
| 2 | 2_8 | 2_8 | CHEM1{[5gn2c6]}\|RNA1{P.[dR](C)P.[dR](A)P.[MOE](A)[sP].[MOE](A)[sP].[LR](A)[PS2].[dR](A)[sP].[dR](G)[sP].[dR] |
| | | | (T)[sP].[dR](T)[sP].[dR](T)[sP].[dR](C)[sP].[dR](T)[sP].[mR](C)[sP].[dR](A)[sP].[dR](G)[sP].[dR](A)[sP].[dR](T)[sP].[d |
| | | | R](A)[sP].[LR](G)[sP].[LR](G)[PS2].[MOE]([5meC])}$CHEM1, RNA1, 1:R2-1:R1$$$V2.0 |
| 2 | 2_9 | 2_9 | CHEM1{[5gn2c6]}\|RNA1{P.[dR](C)P.[dR](A)P.[MOE](A)[sP].[LR](A)[sP].[LR](A)[sP].[dR](A)[P].[dR](G)[sP].[dR](T)[ |
| | | | sP].[dR](T)[sP].[dR](T)[sP].[dR](C)[sP].[dR](T)[sP].[dR](C)[sP].[mR](A)[sP].[dR](G)[sP].[dR](A)[sP].[dR](T)[sP].[dR]( |
| | | | A)[sP].[LR](G)[sP].[LR](G)[sP].[MOE]([5meC])}$CHEM1, RNA1, 1:R2-1:R1$$$V2.0 |
| 2 | 2_10 | 2_10 | CHEM1{[5gn2c6]}\|RNA1{P.[dR](C)P.[dR](A)P.[LR](A)[PS2].[LR](A)[sP].[LR](A)[P].[dR](A)[sP].[R](G)[sP].[dR](T)[ |
| | | | sP].[dR](T)[sP].[dR](T)[sP].[mR](C)[sP].[dR](T)[sP].[dR](C)[sP].[dR](A)[sP].[dR](G)[sP].[dR](A)[sP].[dR](T)[sP].[dR]( |
| | | | A)[sP].[LR](G)[P].[LR](G)[PS2].[LR]([5meC])}$CHEM1, RNA1, 1:R2-1:R1$$$V2.0 |
| 2 | 2_11 | 2_11 | CHEM1{[5gn2c6]}\|RNA1{P.[dR](C)P.[dR](A)P.[MOE](A)[sP].[LR](A)[sP].[LR](A)[sP].[dR](A)[sP].[dR](G)[sP].[dR](T)[ |
| | | | sP].[dR](T)[sP].[dR](T)[sP].[mR](C)[sP].[dR](T)[sP].[dR](C)[sP].[dR](A)[sP].[dR](G)[sP].[dR](A)[sP].[dR](T)[sP].[dR]( |
| | | | A)[sP].[LR](G)[sP].[LR](G)[PS2].[MOE]([5meC])}$CHEM1, RNA1, 1:R2-1:R1$$$V2.0 |
| 2 | 2_12 | 2_12 | CHEM1{[5gn2c6]}\|RNA1{P.[dR](C)P.[dR](A)P.[MOE](A)[sP].[LR](A)[PS2].[LR](A)[sP].[dR](A)[sP].[dR](G)[sP].[dR](T |
| | | | )[sP].[dR](T)[sP].[dR](T)[sP].[dR](C)[sP].[dR](T)[sP].[dR](C)[sP].[dR](A)[sP].[dR](G)[sP].[dR](A)[sP].[dR](T)[sP].[mR] |
| | | | (A)[sP].[LR](G)[sP].[LR](G)[PS2].[MOE]([5meC])}$CHEM1, RNA1, 1:R2-1:R1$$$V2.0 |
| 17 | 17_1 | 17_1 | CHEM1{[5gn2c6]}\|RNA1{P.[dR](C)P.[dR](A)P.[MOE](A)[sP].[MOE](A)[sP].[LR](A)[sP].[dR](A)[sP].[dR](G)[sP].[dR]( |
| | | | T)[sP].[dR](T)[sP].[mR](U)[sP].[dR](C)[sP].[dR](T)[sP].[dR](C)[sP].[dR](A)[sP].[dR](G)[sP].[dR](A)[sP].[dR](T)[sP].[d |
| | | | R](A)[sP].[LR](G)[sP].[LR](G)[PS2].[MOE]([5meC])}$CHEM1, RNA1, 1:R2-1:R1$$$V2.0 |
| 18 | 18_1 | 18_1 | CHEM1{[5gn2c6]}\|RNA1{P.[dR](C)P.[dR](A)P.[LR](A)[PS2].[LR](G)[sP].[dR](G)[sP].[dR](C)[sP].[LR](A)[sP].[dR](T)[ |
| | | | sP].[dR](T)[sP].[dR](A)[sP].[dR](T)[sP].[dR](A)[sP].[dR](G)[sP].[dR](G)[sP].[mR](U)[sP].[dR](T)[sP].[dR](T)[sP].[LR]( |
| | | | T)[sP].[LR](T)[PS2].[LR]([5meC])}$CHEM1, RNA1, 1:R2-1:R1$$$V2.0 |
| 19 | 19_1 | 19_1 | CHEM1{[5gn2c6]}\|RNA1{P.[dR](C)P.[dR](A)P.[LR](A)[PS2].[LR](G)[sP].[dR](G)[P].[dR](C)[sP].[LR](A)[sP].[mR](U) |
| | | | [sP].[dR](T)[sP].[dR](A)[sP].[dR](T)[sP].[dR](A)[sP].[dR](G)[sP].[dR](G)[sP].[dR](T)[sP].[dR](T)[sP].[dR](T)[sP].[LR]( |
| | | | T)[sP].[LR](T)[PS2].[LR]([5meC])}$CHEM1, RNA1, 1:R2-1:R1$$$V2.0 |
| 20 | 20_1 | 20_1 | CHEM1{[5gn2c6]}\|RNA1{P.[dR](C)P.[dR](A)P.[LR](G)[PS2].[LR]([5meC])[sP].[dR](A)[sP].[dR](T)[sP].[dR](T)[sP].[d |
| | | | R](A)[sP].[dR](T)[sP].[dR](A)[sP].[dR](G)[sP].[dR](G)[sP].[dR](T)[sP].[LR](T)[sP].[dR](T)[sP].[dR](T)[sP].[dR](T)[sP].[ |
| | | | dR](C)[sP].[LR](T)[PS2].[LR]([5meC])}$CHEM1, RNA1, 1:R2-1:R1$$$V2.0 |
| 20 | 20_2 | 20_2 | CHEM1{[5gn2c6]}\|RNA1{P.[dR](C)P.[dR](A)P.[LR](G)[PS2].[LR]([5meC])[P].[mR](A)[sP].[dR](T)[sP].[dR](T)[sP].[d |
| | | | R](A)[sP].[dR](T)[sP].[dR](A)[sP].[dR](G)[sP].[dR](G)[sP].[dR](T)[sP].[LR](T)[sP].[dR](T)[sP].[dR](T)[P].[dR](T)[sP].[ |
| | | | dR](C)[sP].[LR](T)[PS2].[LR]([5meC])}$CHEM1, RNA1, 1:R2-1:R1$$$V2.0 |
| 20 | 20_3 | 20_3 | CHEM1{[5gn2c6]}\|RNA1{P.[dR](C)P.[dR](A)P.[LR](G)[PS2].[LR]([5meC])[sP].[dR](A)[sP].[dR](T)[sP].[dR](T)[sP].[d |
| | | | R](A)[sP].[dR](T)[sP].[mR](A)[sP].[dR](G)[sP].[dR](G)[sP].[dR](T)[sP].[LR](T)[sP].[dR](T)[sP].[dR](T)[sP].[dR](T)[sP]. |
| | | | [dR](C)[sP].[LR](T)[PS2].[LR]([5meC])} $CHEM1, RNA1, 1:R2-1:R1$$$V2.0 |
| 20 | 20_4 | 20_4 | CHEM1{[5gn2c6]}\|RNA1{P.[dR](C)P.[dR](A)P.[LR](G)[PS2].[LR]([5meC])[sP].[dR](A)[P].[dR](T)[sP].[dR](T)[sP].[d |
| | | | R](A)[sP].[dR](T)[sP].[dR](A)[sP].[mR](G)[sP].[dR](G)[sP].[dR](T)[sP].[LR](T)[P].[dR](T)[sP].[dR](T)[sP].[dR](T)[sP]. |
| | | | [dR](C)[sP].[LR](T)[PS2].[LR]([5meC])}$CHEM1, RNA1, 1:R2-1:R1$$$V2.0 |
| 20 | 20_5 | 20_5 | CHEM1{[5gn2c6]}\|RNA1{P.[dR](C)P.[dR](A)P.[LR](G)[PS2].[LR]([5meC])[sP].[dR](A)[sP].[dR](T)[sP].[dR](T)[sP].[d |
| | | | R](A)[sP].[dR](T)[sP].[dR](A)[sP].[dR](G)[sP].[dR](G)[sP].[dR](T)[sP].[LR](T)[sP].[dR](T)[sP].[dR](T)[sP].[dR](T)[sP].[ |
| | | | dR](C)[sP].[LR](T)[PS2].[MOE]([5meC])}$CHEM1, RNA1, 1:R2-1:R1$$$V2.0 |
| 20 | 20_6 | 20_6 | CHEM1{[5gn2c6]}\|RNA1{P.[dR](C)P.[dR](A)P.[LR](G)[PS2].[LR]([5meC])[sP].[dR](A)[sP].[dR](T)[sP].[dR](T)[sP].[d |
| | | | R](A)[sP].[dR](T)[sP].[dR](A)[sP].[dR](G)[sP].[mR](G)[sP].[dR](T)[sP].[LR](T)[sP].[dR](T)[sP].[dR](T)[sP].[dR](T)[sP]. |
| | | | [dR](C)[sP].[LR](T)[PS2].[LR]([5meC])}$CHEM1, RNA1, 1:R2-1:R1$$$V2.0 |
| 6 | 6_2 | 6_2 | CHEM1{[5gn2c6]}\|RNA1{P.[dR](C)P.[dR](A)P.[LR](G)[PS2].[LR]([5meC])[sP].[LR](A)[sP].[dR](T)[sP].[dR](T)[sP].[d |
| | | | R](A)[sP].[dR](T)[sP].[dR](A)[sP].[dR](G)[sP].[dR](G)[sP].[dR](T)[sP].[dR](T)[sP].[dR](T)[P].[LR](T)[sP].[dR](T)[sP].[ |
| | | | LR]([5meC])[PS2].[LR](T)}$CHEM1, RNA1, 1:R2-1:R1$$$V2.0 |
| 6 | 6_3 | 6_3 | CHEM1{[5gn2c6]}\|RNA1{P.[dR](C)P.[dR](A)P.[LR](G)[PS2].[LR]([5meC])[sP].[LR](A)[sP].[dR](T)[sP].[dR](T)[sP].[d |
| | | | R](A)[sP].[dR](T)[sP].[mR](A)[sP].[dR](G)[sP].[dR](G)[sP].[dR](T)[sP].[dR](T)[sP].[dR](T)[sP].[LR](T)[sP].[dR](T)[sP]. |
| | | | [LR]([5meC])[PS2].[LR](T)}$CHEM1, RNA1, 1:R2-1:R1$$$V2.0 |
| 6 | 6_4 | 6_4 | CHEM1{[5gn2c6]}\|RNA1{P.[dR](C)P.[dR](A)P.[LR](G)[PS2].[LR]([5meC])[sP].[LR](A)[P].[dR](T)[sP].[dR](T)[sP].[d |
| | | | R](A)[sP].[dR](T)[sP].[dR](A)[sP].[mR](G)[sP].[dR](G)[sP].[dR](T)[sP].[dR](T)[sP].[dR](T)[sP].[LR](T)[sP].[dR](T)[sP]. |
| | | | LR]([5meC])[PS2].[LR](T)}$CHEM1, RNA1, 1:R2-1:R1$$$V2.0 |
| 6 | 6_5 | 6_5 | CHEM1{[5gn2c6]}\|RNA1{P.[dR](C)P.[dR](A)P.[LR](G)[PS2].[LR]([5meC])[sP].[mR](A)[sP].[dR](T)[sP].[dR](T)[sP].[d |
| | | | R](A)[sP].[dR](T)[sP].[dR](A)[sP].[dR](G)[sP].[dR](G)[sP].[dR](T)[sP].[dR](T)[sP].[dR](T)[sP].[LR](T)[sP].[LR](T)[sP].[ |
| | | | LR]([5meC])[PS2].[LR](T)}$CHEM1, RNA1, 1:R2-1:R1$$$V2.0 |
| 6 | 6_6 | 6_6 | CHEM1{[5gn2c6]}\|RNA1{P.[dR](C)P.[dR](A)P.[LR](G)[PS2].[LR]([5meC])[sP].[dR](A)[sP].[dR](T)[sP].[dR](T)[sP].[d |
| | | | R](A)[sP].[dR](T)[sP].[mR](A)[sP].[dR](G)[sP].[dR](G)[sP].[dR](T)[sP].[dR](T)[sP].[dR](T)[sP].[LR](T)[sP].[LR](T)[sP]. |
| | | | [LR]([5meC])[PS2].[LR](T)}$CHEM1, RNA1, 1:R2-1:R1$$$V2.0 |
| 6 | 6_7 | 6_7 | CHEM1{[5gn2c6]}\|RNA1{P.[dR](C)P.[dR](A)P.[LR](G)[PS2].[LR]([5meC])[sP].[dR](A)[sP].[dR](T)[sP].[dR](T)[sP].[d |
| | | | R](A)[sP].[dR](T)[sP].[dR](A)[sP].[mR](G)[sP].[dR](G)[sP].[dR](T)[sP].[dR](T)[sP].[dR](T)[sP].[LR](T)[sP].[LR](T)[sP]. |
| | | | [LR]([5meC])[PS2].[LR](T)}$CHEM1, RNA1, 1:R2-1:R1$$$V2.0 |
| 6 | 6_8 | 6_8 | CHEM1{[5gn2c6]}\|RNA1{P.[dR](C)P.[dR](A)P.[LR](G)[PS2].[LR]([5meC])[sP].[dR](A)[sP].[dR](T)[sP].[dR](T)[sP].[d |
| | | | R](A)[sP].[dR](T)[sP].[dR](A)[sP].[dR](G)[sP].[mR](G)[sP].[dR](T)[sP].[dR](T)[sP].[dR](T)[sP].[LR](T)[sP].[LR](T)[sP]. |
| | | | [LR]([5meC])[PS2].[LR](T)}$CHEM1, RNA1, 1:R2-1:R1$$$V2.0 |
| 6 | 6_9 | 6_9 | CHEM1{[5gn2c6]}\|RNA1{P.[dR](C)P.[dR](A)P.[MOE](G)[sP].[LR]([5meC])[PS2].[dR](A)[sP].[dR](T)[sP].[dR](T)[P].[ |
| | | | dR](A)[sP].[dR](T)[sP].[dR](A)[sP].[dR](G)[sP].[dR](G)[sP].[dR](T)[sP].[dR](T)[sP].[dR](T)[sP].[LR](T)[sP].[LR](T)[sP] |
| | | | .[LR]([5meC])[PS2].[MOE](T)}$CHEM1, RNA1, 1:R2-1:R1$$$V2.0 |
| 6 | 6_10 | 6_10 | CHEM1{[5gn2c6]}\|RNA1{P.[dR](C)P.[dR](A)P.[MOE](G)[P].[LR]([5meC])[PS2].[dR](A)[sP].[dR](T)[sP].[dR](T)[sP].[ |
| | | | dR](A)[sP].[dR](T)[sP].[dR](A)[sP].[dR](G)[sP].[dR](G)[P].[dR](T)[sP].[dR](T)[sP].[dR](T)[sP].[LR](T)[P].[LR](T)[PS |
| | | | 2].[MOE]([5meC])[sP].[MOE](T)}$CHEM1, RNA1, 1:R2-1:R1$$$V2.0 |
| 6 | 6_11 | 6_11 | CHEM1{[5gn2c6]}\|RNA1{P.[dR](C)P.[dR](A)P.[MOE](G)[sP].[LR]([5meC])[PS2].[dR](A)[sP].[dR](T)[sP].[dR](T)[sP].[ |
| | | | dR](A)[sP].[dR](T)[sP].[dR](A)[P].[dR](G)[sP].[dR](G)[sP].[dR](T)[sP].[dR](T)[sP].[dR](T)[sP].[LR](T)[sP].[LR](T)[PS |
| | | | 2].[LR]([5meC])[PS2].[MOE](T)}$CHEM1,RNA1, 1:R2-1:R1$$$V2.0 |
| 6 | 6_12 | 6_12 | CHEM1{[5gn2c6]}\|RNA1{P.[dR](C)P.[dR](A)P.[MOE](G)[P].[LR]([5meC])[PS2].[dR](A)[sP].[dR](T)[sP].[dR](T)[sP].[ |
| | | | dR](A)[sP].[dR](T)[sP].[dR](A)[sP].[dR](G)[sP].[dR](G)[sP].[dR](T)[sP].[dR](T)[sP].[dR](T)[sP].[LR](T)[PS2].[LR](T)[P |
| | | | S2].[MOE]([5meC])[P].[MOE](T)}$CHEM1, RNA1, 1:R2-1:R1$$$V2.0 |
| 21 | 21_1 | 21_1 | CHEM1{[5gn2c6]}\|RNA1{P.[dR](C)P.[dR](A)P.[LR](G)[PS2].[LR]([5meC])[sP].[dR](A)[sP].[dR](T)[sP].[dR](T)[sP].[d |
| | | | R](A)[sP].[dR](T)[sP].[dR](A)[sP].[dR](G)[sP].[dR](G)[sP].[dR](T)[sP].[LR](T)[sP].[dR](T)[sP].[dR](T)[sP].[mR](U)[sP] |
| | | | .[dR](C)[sP].[LR](T)[PS2].[LR]([5meC])}$CHEM1, RNA1, 1:R2-1:R1$$$V2.0 |
| 22 | 22_1 | 22_1 | CHEM1{[5gn2c6]}\|RNA1{P.[dR](C)P.[dR](A)P.[LR](G)[PS2].[LR]([5meC])[sP].[dR](A)[sP].[dR](T)[sP].[dR](T)[sP].[d |
| | | | R](A)[sP].[dR](T)[P].[dR](A)[sP].[dR](G)[sP].[dR](G)[sP].[dR](T)[sP].[LR](T)[sP].[dR](T)[sP].[mR](U)[sP].[dR](T)[sP] |
| | | | .[dR](C)[sP].[LR](T)[PS2].[LR]([5meC])}$CHEM1, RNA1, 1:R2-1:R1$$$V2.0 |
| 23 | 23_1 | 23_1 | CHEM1{[5gn2c6]}\|RNA1{P.[dR](C)P.[dR](A)P.[LR](G)[PS2].[LR]([5meC])[sP].[dR](A)[sP].[dR](T)[sP].[dR](T)[sP].[d |
| | | | R](A)[sP].[dR](T)[sP].[dR](A)[sP].[dR](G)[sP].[dR](G)[sP].[dR](T)[sP].[LR](T)[sP].[mR](U)[sP].[dR](T)[sP].[dR](T)[sP] |
| | | | .[dR](C)[sP].[LR](T)[PS2].[LR]([5meC])}$CHEM1, RNA1, 1:R2-1:R1$$$V2.0 |
| 24 | 24_1 | 24_1 | CHEM1{[5gn2c6]}\|RNA1{P.[dR](C)P.[dR](A)P.[LR](G)[PS2].[LR]([5meC])[sP].[dR](A)[sP].[dR](T)[sP].[dR](T)[sP].[d |
| | | | R](A)[sP].[dR](T)[sP].[dR](A)[sP].[dR](G)[sP].[dR](G)[P].[dR](T)[sP].[dR](T)[sP].[mR](U)[sP].[LR](T)[sP].[LR](T)[sP] |
| | | | .[LR]([5meC])[PS2].[LR](T)}$CHEM1, RNA1, 1:R2-1:R1$$$V2.0 |
| 25 | 25_1 | 25_1 | CHEM1{[5gn2c6]}\|RNA1{P.[dR](C)P.[dR](A)P.[LR](G)[PS2].[LR]([5meC])[sP].[LR](A)[sP].[dR](T)[sP].[dR](T)[sP].[d |
| | | | R](A)[sP].[dR](T)[sP].[dR](A)[sP].[dR](G)[sP].[dR](G)[sP].[dR](T)[sP].[mR](U)[sP].[dR](T)[sP].[LR](T)[sP].[dR](T)[sP] |
| | | | .[LR]([5meC])[PS2].[LR](T)}$CHEM1, RNA1, 1:R2-1:R1$$$V2.0 |
| 25 | 25_2 | 25_2 | CHEM1{[5gn2c6]}\|RNA1{P.[dR](C)P.[dR](A)P.[LR](G)[PS2].[LR]([5meC])[sP].[dR](A)[sP].[dR](T)[sP].[dR](T)[sP].[d |
| | | | R](A)[sP].[dR](T)[sP].[dR](A)[sP].[dR](G)[sP].[dR](G)[sP].[dR](T)[sP].[mR](U)[sP].[dR](T)[sP].[LR](T)[sP].[LR](T)[sP] |
| | | | .[LR]([5meC])[PS2].[LR](T)}$CHEM1, RNA1, 1:R2-1:R1$$$V2.0 |
| 26 | 26_1 | 26_1 | CHEM1{[5gn2c6]}\|RNA1{P.[dR](C)P.[dR](A)P.[LR](G)[PS2].[LR]([5meC])[sP].[dR](A)[sP].[dR](T)[sP].[dR](T)[sP].[d |
| | | | R](A)[sP].[dR](T)[sP].[dR](A)[sP].[dR](G)[sP].[dR](G)[sP].[mR](U)[sP].[LR](T)[sP].[dR](T)[sP].[dR](T)[sP].[dR](T)[sP] |
| | | | .[dR](C)[P].[LR](T)[PS2].[LR]([5meC])}$CHEM1, RNA1, 1:R2-1:R1$$$V2.0 |
| 27 | 27_1 | 27_1 | CHEM1{[5gn2c6]}\|RNA1{P.[dR](C)P.[dR](A)P.[LR](G)[PS2].[LR]([5meC])[sP].[LR](A)[sP].[dR](T)[sP].[dR](T)[sP].[d |
| | | | R](A)[sP].[dR](T)[sP].[dR](A)[sP].[dR](G)[sP].[dR](G)[sP].[mR](U)[sP].[dR](T)[P].[dR](T)[sP].[LR](T)[sP].[dR](T)[sP] |
| | | | .[LR]([5meC])[PS2].[LR](T)}$CHEM1, RNA1, 1:R2-1:R1$$$V2.0 |
| 27 | 27_2 | 27_2 | CHEM1{[5gn2c6]}\|RNA1{P.[dR](C)P.[dR](A)P.[LR](G)[PS2].[LR]([5meC])[sP].[dR](A)[sP].[dR](T)[sP].[dR](T)[sP].[d |
| | | | R](A)[sP].[dR](T)[sP].[dR](A)[sP].[dR](G)[sP].[dR](G)[sP].[mR](U)[sP].[dR](T)[sP].[dR](T)[sP].[LR](T)[sP].[LR](T)[sP] |
| | | | .[LR]([5meC])[PS2].[LR](T)}$CHEM1, RNA1, 1:R2-1:R1$$$V2.0 |
| 28 | 28_1 | 28_1 | CHEM1{[5gn2c6]}\|RNA1{P.[dR](C)P.[dR](A)P.[LR](G)[PS2].[LR]([5meC])[sP].[dR](A)[sP].[dR](T)[sP].[dR](T)[sP].[d |
| | | | R](A)[P].[mR](U)[sP].[dR](A)[sP].[dR](G)[sP].[dR](G)[P].[dR](T)[sP].[dR](T)[sP].[dR](T)[sP].[LR](T)[sP].[LR](T)[sP] |
| | | | .[LR]([5meC])[PS2].[LR](T)}$CHEM1, RNA1, 1:R2-1:R1$$$V2.0 |
| 29 | 29_1 | 29_1 | CHEM1{[5gn2c6]}\|RNA1{P.[dR](C)P.[dR](A)P.[LR](G)[PS2].[LR]([5meC])[sP].[dR](A)[sP].[dR](T)[sP].[mR](U)[sP].[d |
| | | | R](A)[sP].[dR](T)[sP].[dR](A)[sP].[dR](G)[sP].[dR](G)[sP].[dR](T)[P].[dR](T)[sP].[dR](T)[sP].[LR](T)[sP].[LR](T)[sP].[ |
| | | | LR]([5meC])[PS2].[LR](T)}$CHEM1, RNA1, 1:R2-1:R1$$$V2.0 |
| 30 | 30_1 | 30_1 | CHEM1{[5gn2c6]}\|RNA1{P.[dR](C)P.[dR](A)P.[LR](G)[PS2].[LR]([5meC])[P].[dR](A)[P].[mR](U)[sP].[dR](T)[sP].[d |
| | | | R](A)[sP].[dR](T)[sP].[dR](A)[sP].[dR](G)[sP].[dR](G)[sP].[dR](T)[sP].[dR](T)[sP].[dR](T)[sP].[LR](T)[P].[LR](T)[sP].[ |
| | | | LR]([5meC])[PS2].[LR](T)}$CHEM1, RNA1, 1:R2-1:R1$$$V2.0 |
| 11 | 11_2 | 11_2 | CHEM1{[5gn2c6]}\|RNA1{P.[dR](C)P.[dR](A)P.[LR](G)[sP].[LR](T)[sP].[LR](T)[sP].[dR](T)[sP].[dR](T)[sP].[dR](A)[sP |
| | | | ].[dR](A)[sP].[mR](G)[sP].[dR](A)[sP].[dR](T)[sP].[dR](T)[sP].[dR](G)[P].[dR](T)[sP].[dR](G)[sP].[dR](G)[sP].[dR](T)[ |
| | | | sP].[dR](G)[sP].[LR](T)[sP].[LR](T)[sP].[LR](T)}$CHEM1, RNA1, 1:R2-1:R1$$$V2.0 |
| 11 | 11_3 | 11_3 | CHEM1{[5gn2c6]}\|RNA1{P.[dR](C)P.[dR](A)P.[LR](G)[sP].[LR](T)[sP].[LR](T)[sP].[dR](T)[sP].[dR](T)[sP].[dR](A)[sP |
| | | | ].[dR](A)[sP].[mR](G)[sP].[dR](A)[sP].[dR](T)[sP].[dR](T)[sP].[dR](G)[sP].[dR](T)[sP].[dR](G)[sP].[dR](G)[sP].[dR](T)[ |
| | | | sP].[dR](G)[sP].[LR](T)[sP].[LR](T)[PS2].[LR](T)}$CHEM1, RNA1, 1:R2-1:R1$$$V2.0 |

### Example 2—A1CF mRNA Knockdown

A1CF mRNA expression was suppressed in HepaRG cells (a hepatic cell line) using antisense oligonucleotides (ASOs). The effect on the amount of A1CF mRNA was determined using quantitative PCR.

TABLE 6
Materials for A1CF knockdown

| Material | Source | Cat. No./ID |
|---|---|---|
| HepaRG cells | Roche RNCB | RNCB ID: CL007104 |
| | | Specimen: |
| | | CLHO00141 |
| HepaRG growth medium: | | |
| William's E medium | Sigma | 4128 |
| Additives | Biopredic | ADD710c |
| 1x Glutamax | Gibco | 35050-38 |
| HepaRG diff medium: | | |
| William's E Medium | Sigma | 4128 |
| +Diff Medium Supplement with pen/strep | Biopredics | ADD720 |
| DMSO, 1% (v/v) GlutaMAX-I | | |
| PBS | Gibco | #14190-094 |
| Trypsin-EDTA | Sigma | #T4049 |
| 96-well Nunc plate (Nunclon delta) (96-well cell | Thermo Scientific | 167008 |
| culture plate) | | |
| 96-well Oligo plates w barcodes | Eppendorf | 0030609204 |
| Buffer RLT (cell lysis buffer) | Qiagen | 79216 |
| 70% ethanol | Prepared at RICC | — |
| RNeasy 96 plate (filter plate) | Qiagen | 74182 |
| S-block (receiver plate) | Qiagen | 19585 |
| Buffer RW1 (first wash buffer) | Qiagen | 74182 |
| Buffer RPE (second and third wash buffer) | Qiagen | 74182 |
| 96-well PCR plate (elution plate) | Thermo Scientific | AB0900 |
| RNase-free water | Qiagen | 74182 |
| Plate sealer | HJ Bioanalytik | — |
| Airpore tape (sealing tape) | Qiagen | 19571 |
| RNA dilution plate | Thermo Scientific | #AB0900 |
| qScript ™ XLT One-Step RT-qPCR ToughMix ®, | Quanta Bioscience | 95134-500 |
| Low ROX ™ (assay buffer) | | |
| A1CF (Gene Of Interest qPCR assay - probe 1. | IDT | Hs.PT.58.26619649 |
| FAM, Ratio 2) | | |
| POLR2A (Endogenous control qPCR assay. | IDT | Hs.PT.39a.19639531 |
| HEX, Ratio 2) | | |
| MicroAmp Optical 384-well plate (qPCR plate) | Applied Biosystems | 4309849 |
| MicroAmp Optical Adhesive Film | Applied Biosystems | 4311971 |
| Viia7 or Quantstudio 7 (qPCR machine) | Thermo Scientific | — |

HepaRG Cell Culture

HepaRG cells were cultured at 37° C. in a humidified atmosphere with 5% CO₂ in complete HepaRG growth medium consisting of William's E Medium (Sigma), Growth Medium Supplement (Biopredics, Cat #ADD710), 1% (v/v) GlutaMAX-I.

To initiate differentiation, HepaRG cells were grown in complete HepaRG growth medium for two weeks in a T175 flask (after splitting 1:5), until they were fully confluent. The medium was then changed to 50% complete HepaRG growth medium+50% HepaRG diff medium containing 0.9% (v/v) DMSO for 3 days, and later changed to complete HepaRG growth medium+1.8% (v/v) DMSO. The cells need to differentiate in differentiation medium (with 0.9-1.8% DMSO) for 2-3 weeks with medium renewal every 2-3 times per week. Differentiated HepaRG cells (dHepaRG) displayed hepatocyte-like cell islands surrounded by monolayer of biliary-like cells.

Differentiated HepaRG cells were seeded into collagen I coated 96-well plates at 80,000 cells per well in complete HepaRG diff medium. Cells were allowed to recover their differentiated phenotype in 96-well plates for approximately 4-6 days after plating prior to ASO treatment. Normally, one T175 flask of differentiated HepaRG cells contains enough cells for (2-4) 96-well plates seeded with 80,000 cells/well.

The cell culture plates were incubated for 5 days with ASOs and subsequently harvested and processed for RNA extraction.

RNA Extraction

The cell medium was removed from the culture wells by pipetting, then RNA was purified from the cells using the following protocol:

1. Add 125 μL of Buffer RLT to each well of the microplate. Shake it vigorously back and forth for 10 s.

2. Add 1 volume (125 μL) of 70% ethanol to each microplate well and mix well.

3. Place an RNeasy® 96 plate on the top of a S-Block, and apply the samples from step 2 into the wells of the RNeasy 96 plate.

3. Seal the plate with airpore tape and centrifuge at 6000 rpm (˜5300×g) for 5 min. at room temperature. Discard flow-through.

4. Add 800 μL of Buffer RW1 to each well of the RNeasy® 96 plate. Seal the plate with airpore tape and centrifuge at 6000 rpm for 5 min. Discard flow-through and replace with a clean S-Block.

5. Add 800 μL Buffer RPE to each well of the RNeasy® 96 plate. Seal the plate with airpore tape and centrifuge at 6000 rpm for 5 min. at RT. Discard flow-through.

6. Add another 800 μL of Buffer RPE to each well of the RNeasy® 96 plate. Seal the plate with airpore tape and centrifuge at 6000 rpm for 5 min. at RT. Discard flow-through and replace with a clean S-Block.

7. Seal the plate with airpore tape and centrifuge the RNeasy® 96 plate for an additional 10 minutes at 6000 rpm.

8. To elute RNA, add 100 μL of RNase-free water to each well. Immediately after adding water, place a PCR 96-well plate on the s-block and put the filterplate back on top of the PCR plate. Incubate for 2 min. at RT. Then seal the plate with airpore tape and centrifuge at 6000 rpm for 5 min. at RT. Discard the filterplate and seal the PCR plate.

The purified RNA was stored at −80° C. until use.

qPCR Assay

The purified RNA was heat shocked for 40 seconds at 90° C. to melt RNA:ASO duplexes, moved directly to ice and spun down before use.

qPCR standard curves were included as a performance control. Purified RNA from two PBS wells was diluted 5-fold in RNase free water and a dilution series was created from this material by serial dilution in RNase free water.

The qPCR assay was carried out using the following protocol:

1. Prepare the following master mix for each well of a 384-well qPCR plate.
1 reaction (10 uL):
XLT One-Step mix 5 uL
GOI taqman probe (FAM) 0.5 uL
Control taqman probe (VIC) 0.5 uL
Mix the master mix thoroughly before use as the XLT buffer is viscous.

2. Add 6 uL master mix per well in a 384 plate.

3. Add 4 uL of diluted RNA per well from the RNA dilution plate. Seal the 384-well plate and vortex. Use a plate or other plastic between vortex rubber and the 384 plate to avoid dark rubber on wells. Spin down the qPCR plate for 3 mins at max speed.

4. Run the following program on a ViiA7 or Quantstudio 7 qPCR instrument:
15 minutes at 50° C.
3 minutes at 95° C.
Followed by 40 cycles of:
95° C. for 5 secs
60° C. for 45 secs
Set temperature change rate to 1.9° C./sec for all steps, except 95 to 60 which is 1.6° C./sec

qPCR Data Analysis

The qPCR data was captured and raw data quality control done in Quantstudio 7 software.

1. Quantity was calculated by the delta delta Ct method (Quantity=2\hat{ }(−Ct)*1000000000)

2. Quantity was normalized to the calculated quantity for the housekeeping gene assay run in the same well. Relative Target Quantity=QUANTITY_target/QUANTITY_housekeeping

3. The RNA knockdown was calculated for each well by division with the mean of all PBS-treated wells on the same plate. Normalised Target Quantity=(Relative Target Quantity/[mean] Relative Target Quantity]_pbs_wells)*100

4. For concentration-response experiments, a curve was fitted from the RNA knockdown values (step 3-4) for each compound. Curves are fitted using a 4 Parameter Sigmoidal Dose-Response Model.

The final data are shown in Table 7 below as a percentage of untreated (PBS) wells.

TABLE 7
Results of A1CF knockdown

| SEQ | CMP | EC50 (μM) | A1CF mRNA remaining (%) | |
|---|---|---|---|---|
| ID NO | ID NO | A1CF | 1 μM ASO | 5 μM ASO |
| 1 | 1_1 | 0.10358 | 19.72 | 1.69 |
| 2 | 2_1 | 0.17106 | 17.28 | 3.02 |
| 3 | 3_1 | 0.18750 | 36.91 | 7.20 |
| 4 | 4_1 | 0.21593 | 19.97 | 3.36 |
| 4 | 4_2 | 0.15548 | 21.60 | 2.11 |
| 5 | 5_1 | 0.20544 | 24.45 | 1.01 |
| 5 | 5_1 | 0.21824 | 30.57 | 2.39 |
| 6 | 6_1 | 0.09931 | 20.16 | 1.13 |
| 7 | 7_1 | 0.06919 | 30.59 | 2.28 |
| 7 | 7_2 | 0.06142 | 17.24 | 1.24 |
| 8 | 8_1 | 0.12850 | 4.42 | 0.55 |
| 2 | 2_2 | 0.20129 | 16.90 | 3.03 |
| 9 | 9_1 | 0.23145 | 30.34 | 9.36 |
| 10 | 10_1 | 0.18685 | 10.75 | 1.54 |
| 11 | 11_1 | 0.11215 | 28.79 | 4.51 |
| 4 | 4_3 | 0.15581 | 35.34 | 6.42 |
| 12 | 12_1 | 0.20495 | 25.92 | 2.41 |
| 5 | 5_3 | 0.23966 | 24.41 | 1.21 |
| 13 | 13_1 | 0.17192 | 30.21 | 1.25 |
| 14 | 14_1 | 0.23067 | 28.61 | 1.43 |
| 15 | 15_1 | ND | 14.63 | 1.47 |
| 16 | 16_1 | ND | 14.12 | 1.67 |
| 16 | 16_2 | ND | 22.00 | 3.05 |
| 16 | 16_3 | ND | 14.74 | 1.80 |
| 2 | 2_3 | ND | 25.70 | 3.24 |
| 2 | 2_4 | ND | 13.73 | 3.45 |
| 2 | 2_5 | ND | 15.90 | 1.67 |
| 2 | 2_6 | ND | 17.33 | 5.55 |
| 2 | 2_7 | ND | 12.88 | 1.63 |
| 2 | 2_8 | ND | 17.26 | 3.09 |
| 2 | 2_9 | ND | 25.20 | 4.10 |
| 2 | 2_10 | ND | 13.01 | 1.44 |
| 2 | 2_11 | ND | 20.78 | 3.64 |
| 2 | 2_12 | ND | 19.59 | 2.45 |
| 17 | 17_1 | ND | 23.10 | 3.99 |
| 18 | 18_1 | ND | 7.79 | 0.61 |
| 19 | 19_1 | ND | 11.77 | 0.86 |
| 20 | 20_1 | ND | 7.51 | 0.58 |
| 20 | 20_2 | ND | 8.79 | 0.48 |
| 20 | 20_3 | ND | 12.00 | 1.04 |
| 20 | 20_4 | ND | 14.23 | 2.41 |
| 20 | 20_5 | ND | 9.97 | 0.60 |
| 20 | 20_6 | ND | 16.13 | 3.22 |
| 6 | 6_2 | ND | 8.50 | 0.32 |
| 6 | 6_3 | ND | 12.05 | 0.70 |
| 6 | 6_4 | ND | 10.41 | 0.77 |
| 6 | 6_5 | ND | 5.11 | 0.14 |
| 6 | 6_6 | ND | 6.61 | 0.27 |
| 6 | 6_7 | ND | 8.99 | 0.65 |
| 6 | 6_8 | ND | 9.80 | 0.58 |
| 6 | 6_9 | ND | 9.07 | 0.33 |
| 6 | 6_10 | ND | 15.85 | 0.63 |
| 6 | 6_11 | ND | 5.04 | 0.19 |
| 6 | 6_12 | ND | 5.01 | 0.23 |
| 21 | 21_1 | ND | 6.93 | 0.51 |
| 22 | 22_1 | ND | 12.64 | 1.29 |
| 23 | 23_1 | ND | 7.82 | 0.54 |
| 24 | 24_1 | ND | 17.03 | 0.72 |
| 25 | 25_1 | ND | 11.31 | 0.52 |
| 25 | 25_2 | ND | 8.60 | 0.31 |
| 26 | 26_1 | ND | 12.49 | 1.03 |
| 27 | 27_1 | ND | 12.92 | 0.71 |
| 27 | 27_2 | ND | 9.07 | 0.49 |
| 28 | 28_1 | ND | 10.91 | 0.77 |
| 29 | 29_1 | ND | 15.88 | 2.34 |
| 30 | 30_1 | ND | 15.57 | 0.71 |
| 11 | 11_2 | ND | 43.11 | 10.36 |
| 11 | 11_3 | ND | 19.91 | 3.57 |

ND = not determined

### Example 3—Potency and Antiviral Efficacy of ASOs in Primary Human Hepatocytes

Primary human hepatocytes (PHH) were treated with ASOs targeting A1CF mRNA. The effect of these treatments on A1CF mRNA expression, pgRNA expression, HBV DNA secretion into the supernatant and total intracellular HBV DNA were then quantified using qPCR. The effect on A1CF protein levels was also quantified using automated Western blotting.

Primary Human Hepatocytes (PHH) Cell Culture

Fresh primary human hepatocytes (PHH) harvested from humanized mice (uPA/SCID mice) were obtained from PhoenixBio Co., Ltd (Japan) in 96-well format and cultured in modified hepatocyte clonal growth medium (dHCGM). dHCGM is a DMEM medium containing 100 U/ml Penicillin, 100 μg/ml Streptomycin, 20 mM Hepes, 44 mM NaHC03, 15 μg/ml L-proline, 0.25 μg/ml Insulin, 50 nM Dexamethazone, 5 ng/ml EGF, 0.1 mM Asc-2P, 2% DMSO and 10% FBS (Ishida et al., 2015). Cells were cultured at 37° C., in a humidified atmosphere with 5% CO2.

PHH were infected with HBV GtD derived from HepG2.2.15 cell culture in the presence of 4% PEG for 16-20 h. The virus inoculum was removed the following day and cells were washed 3 times with PBS before addition of fresh medium. To allow for cccDNA establishment, compound treatment was started at day 4 post HBV infection in triplicates at a final concentration of 25 uM in a total volume of 120 ul per well. The same treatment was repeated on day 11 and 18. On day 7, 14 and 21 the medium was changed. For the cccDNA readout the cells were maintained in medium supplemented with 10 nM entecavir (ETV) starting from day 7 post infection until the day of harvest. All other readouts were conducted without adding ETV.

On day 25, the supernatants were harvested and stored at −80° C. The cells were washed with 1×DPBS (Gibco, #14190250) once using 150 ul/well. For the RNA readout 200 ul/well MagNA Pure 96 External Lysis Buffer (Roche, #06374913001) was added and the plates frozen at −80° C. For the cccDNA and intracellular DNA readout the cells were lysed with 80 ul/well cccDNA SDS lysis buffer (50 mM Tris pH8, 5 mM EDTA, 1% SDS in nuclease free water), then frozen in the −80° C. freezer for 2 hours minimally. For the protein readout the cells are lysed with 100 ul/well RIPA buffer (ThermoFischer, #89901) supplemented with complete Protease Inhibitor Cocktail tablet (Sigma, #11697498001; 1 tablet per 25 ml) and 1 mM Phenylmethylsulfonyl fluoride (Roche, #10837091001) and frozen at −80° C.

A1CF mRNA and pgRNA Quantification—RNA Extraction and RT-qPCR

Total RNA was extracted from the cells using a MagNA Pure robot and the MagNA Pure 96 Cellular RNA Large Volume Kit (Roche, #05467535001) according to the manufacturer's protocol using the “Cellular RNA LV” protocol with a final elution volume of 100 μl. The A1CF mRNA and pgRNA expression levels were quantified in technical duplicates by qPCR using a QuantStudio 12K Flex (Applied Biosystems), the TaqMan RNA-to-CT 1-Step Kit (Applied Biosystems, #4392938), and human GusB endogenous control. Run the qPCR on QuantStudio Cycler with 48° C. for 15 minutes, then 95° C. for 10 minutes, then 40 cycles with 95° C. for 15 seconds and 60° C. for 1 minute, with 10 μl reaction volume. The mRNA expression was analyzed using the comparative cycle threshold 2-ΔΔCt method normalized to the reference gene GusB and to HBV-infected, non-treated cells. Fold change calculations were conducted in GraphPad using each Ct value individually (no averaging of technical Ct values for the dCT calculation). Excel was then used to calculate the average fold change for each biological sample by averaging the values of the technical duplicates. From the fold changes for each biological replicates the percentage, final average, SD and N were then calculated using excel. TaqMan primers used for GusB mRNA, A1CF mRNA and pgRNA quantification are listed in Table 8 below.

TABLE 8
Primers for GusB mRNA, A1CF mRNA and pgRNA quantification

| Target | Source |
|---|---|
| A1CF | ThermoFisher-Assay ID: Hs00205840_m1 |
| pgRNA | ThermoFisher-Custom assay: |
| | Probe FAM GAGGCAGGTCCCCTAGAAGA (SEQ ID NO 54) |
| | Forward GGAGTGTGGATTCGCACTCCT (SEQ ID NO 55) |
| | Reverse AGATTGAGATCTTCTGCGAC (SEQ ID NO 56) |
| GusB | ThermoFisher-Assay ID: Hs00939627_m1 |

The relative A1CF mRNA and pgRNA expression levels in PHH cells are shown in Table 9 as % of control, i.e. the lower the value the larger the inhibition.

TABLE 9
A1CF mRNA and pgRNA expression levels in PHH cells

| SEQ | CNJ | A1CF mRNA (%) | | | pgRNA (%) | | |
|---|---|---|---|---|---|---|---|
| ID NO | ID NO | Mean | SD | N | Mean | SD | N |
| 6 | 6_1 | 0.071 | 0.037 | 3 | 37.08 | 8.05 | 2 |
| 1 | 1_1 | 0.120 | 0.011 | 3 | 13.99 | 1.29 | 3 |
| 7 | 7_1 | 0.093 | 0.031 | 3 | 2.76 | 1.34 | 3 |
| 7 | 7_2 | 0.092 | 0.037 | 3 | 3.08 | 0.00 | 2 |
| 8 | 8_1 | 0.652 | 0.002 | 2 | 2.89 | N/A | 1 |
| 2 | 2_2 | 1.742 | 1.199 | 3 | 48.60 | 17.18 | 3 |
| 2 | 2_1 | 3.359 | 2.148 | 3 | 19.29 | 2.10 | 3 |
| 3 | 3_1 | 0.980 | 0.180 | 3 | 16.09 | 5.70 | 3 |
| 9 | 9_1 | 5.181 | 0.767 | 3 | 134.75 | 7.55 | 3 |
| 10 | 10_1 | 3.157 | 0.288 | 3 | 4.13 | 2.08 | 3 |
| 11 | 11_1 | 0.772 | 0.248 | 3 | 27.11 | 10.30 | 3 |
| 4 | 4_3 | 0.127 | 0.056 | 3 | 81.44 | 23.89 | 3 |
| 4 | 4_1 | 0.230 | 0.084 | 3 | 69.02 | 9.96 | 3 |
| 4 | 4_2 | 0.243 | 0.095 | 3 | 99.31 | 11.96 | 3 |
| 12 | 12_1 | 0.367 | 0.329 | 3 | 31.60 | 27.10 | 3 |
| 5 | 5_3 | 0.396 | 0.002 | 2 | 36.08 | 4.36 | 2 |
| 5 | 5_1 | 0.434 | 0.059 | 3 | 46.90 | 7.28 | 3 |
| 13 | 13_1 | 0.700 | 0.059 | 3 | 28.71 | 5.74 | 3 |
| 14 | 14_1 | 0.274 | 0.016 | 3 | 42.78 | 3.72 | 3 |

Secreted HBV DNA Quantification—Supernatant DNA Extraction and qPCR

DNA was extracted from 25 uL of supernatant on the MagNA Pure robot with the MagNA Pure 96 DNA and Viral NA Small Volume Kit (Roche, #06543588001) using the “Viral NA Plasma ext lys SV 4.0” protocol.

For quantification of HBV DNA a 99 nucleotide fragment covering the core region was amplified with forward primer CTG TGC CTT GGG TGG CTT T (SEQ ID NO 57) (final concentration 200 nM), reverse primer AAG GAA AGA AGT CAG AAG GCA AAA (SEQ ID NO 58) (final concentration 200 nM), and probe 56-FAM-AGC TCC AAA/ZEN/TTC TTT ATA AGG GTC GAT GTC CAT G-3IABkFQ (SEQ ID NO 59) (final concentration 100 nM) (IDT DNA) using the TaqMan Gene Expression Master Mix and the following cycling condition: 2 min at 50° C., 10 min at 95° C., and 40 cycles of 95° C. for 15 sec and 60° C. for 1 min. All qPCR reactions were performed using the QuantStudio 12K Flex Real Time PCR system (Life Technologies).

The relative expression levels were calculated using the comparative cycle threshold 2-ΔCt method normalized to HBV-infected, non-treated cells. Fold change calculations were conducted in GraphPad using each Ct value individually (no averaging of technical Ct values for the dCT calculation). Excel was then used to calculate the average fold change for each biological sample by averaging the values of the technical duplicates. From the fold changes for each biological replicates the percentage, final average, SD and N were then calculated using excel.

TaqMan primers used for secreted HBV DNA quantification are listed in Table 10 below.

TABLE 10
Primers for supernatant HBV quantification

| Target | Source |
|---|---|
| HBV core | forward primer CTG TGC CTT GGG TGG CTT T (SEQ ID NO 57) |
| | reverse primer AAG GAA AGA AGT CAG AAG GCA AAA (SEQ ID NO 58) |
| | probe 56-FAM-AGC TCC AAA/ZEN/TTC TTT ATA AGG GTC GAT GTC CAT |
| | G-3IABKFQ (SEQ ID NO 59) |

The relative secreted HBV DNA levels in PHH cells are shown in Table 11 as % of control, i.e. the lower the value the larger the inhibition.

TABLE 11
Relative secreted HBV DNA levels

| | | |
|---|---|---|
| SEQ | CNJ | Secreted HBV DNA (%) |

| ID NO | ID NO | Mean | SD | N |
|---|---|---|---|---|
| 6 | 6_1 | 32.51 | 4.29 | 3 |
| 1 | 1_1 | 5.63 | 0.68 | 3 |
| 7 | 7_1 | 3.93 | 0.87 | 3 |
| 7 | 7_2 | 8.85 | 1.35 | 3 |
| 8 | 8_1 | 2.85 | 0.26 | 3 |
| 2 | 2_2 | 109.32 | 17.39 | 3 |
| 2 | 2_1 | 58.94 | 33.92 | 3 |
| 3 | 3_1 | 21.07 | 5.33 | 3 |
| 9 | 9_1 | 178.96 | 17.77 | 3 |
| 10 | 10_1 | 9.18 | 3.00 | 3 |
| 11 | 11_1 | 18.80 | 4.46 | 3 |
| 4 | 4_3 | 89.06 | 10.62 | 3 |
| 4 | 4_1 | 93.54 | 6.33 | 3 |
| 4 | 4_3 | 111.12 | 10.90 | 3 |
| 12 | 12_1 | 46.26 | 6.30 | 3 |
| 5 | 5_3 | 51.21 | 18.24 | 3 |
| 5 | 5_1 | 83.29 | 8.91 | 3 |
| 13 | 13_1 | 33.11 | 5.83 | 3 |
| 14 | 14_1 | 69.61 | 3.28 | 3 |

Intracellular Total HBV Quantification—Cellular DNA Extraction and qPCR

Intracellular DNA was extracted using the ZR-96 Genomic DNA Clean & Concentrator-5 kit (Zymo Research, #D4067). First, 1 uL of Proteinase K (Ambion, #AM2546; 20 mg/mL) was added to each thawed lysate and the plates were sealed and incubated at 56° C. for 30 minutes. After incubation, the lysates are mixed with 240 ul ChIP DNA binding buffer and loaded on the Zymo-Spin i-96-XL Plate mounted on a collection plate. The plates are then spun at 3500×g for 5 minutes, flow-through was discarded and 200 uL of DNA Wash buffer added to each well. The plates are then spun at 3500×g for 5 minutes again, discarding the flow-through. The wash step is repeated once, then the Zymo-Spin I-96-XL Plate is mounted on an elution plate and 40 uL of the DNA Elution Buffer is added directly to the matrix in each well. The plates are incubated at RT for 5 minutes, then spun at 3500×g for 5 minutes to elute the DNA. The DNA is either stored at −20° C. or used directly in the intracellular total HBV DNA qPCR. DNA samples were diluted 1:10 with water before running the total HBV DNA qPCR. The total intracellular HBV DNA expression levels were quantified in technical duplicates by qPCR using a QuantStudio 12K Flex (Applied Biosystems), the TaqMan Fast Advanced Master Mix (Applied Biosystems, #4444557) and human HBB endogenous control. Run the qPCR on QuantStudio Cycler with standard settings for Fast heating block (95° C. for 20 seconds, then 40 cycles with 95° C. for 1 second and 60° C. for 20 seconds, 10 ul reaction volume).

For HBB Ct values the threshold was set at 0.18. The DNA expression levels were analyzed using the comparative cycle threshold 2-ΔΔCt method normalized to the reference gene HBB and to HBV-infected, non-treated cells. Fold change calculations were conducted in GraphPad using each Ct value individually (no averaging of technical Ct values for the dCT calculation). Excel was then used to calculate the average fold change for each biological sample by averaging the values of the technical duplicates. From the fold changes for each biological replicates the percentage, final average, SD and N were then calculated using excel. TaqMan primers used for HBB and total HBV DNA quantification are listed in Table 12 below.

TABLE 12
Primers for total HBV DNA and HBB quantification

| Target | Source |
|---|---|
| Total HBV DNA | ThermoFisher - Assay ID: Pa03453406_s1 |
| HBB | ThermoFisher - Assay ID: HBB Hs00758889_s1 |

The total intracellular HBV DNA expression levels in PHH cells are shown in Table 13 as % of control, i.e. the lower the value the larger the inhibition.

TABLE 13
Total intracellular HBV DNA

| | | |
|---|---|---|
| SEQ | CNJ | total intracellular HBV DNA (%) |

| ID NO | ID NO | Mean | SD | N |
|---|---|---|---|---|
| 6 | 6_1 | 41.35 | 10.57 | 3 |
| 1 | 1_1 | 23.06 | 4.47 | 3 |
| 7 | 7_1 | 43.05 | 2.98 | 3 |
| 7 | 7_2 | 36.80 | 11.16 | 3 |
| 8 | 8_1 | 29.64 | 12.58 | 3 |
| 2 | 2_2 | 69.93 | 14.26 | 3 |
| 2 | 2_1 | 35.34 | 5.33 | 3 |
| 3 | 3_1 | 33.07 | 5.72 | 3 |
| 9 | 9_1 | 96.81 | 13.27 | 3 |
| 10 | 10_1 | 80.67 | 27.70 | 3 |
| 11 | 11_1 | 24.26 | 7.21 | 3 |
| 4 | 4_3 | 125.87 | 7.51 | 3 |
| 4 | 4_1 | 104.62 | 15.89 | 3 |
| 4 | 4_3 | 92.86 | 36.32 | 3 |
| 12 | 12_1 | 106.90 | 31.03 | 3 |
| 5 | 5_3 | 99.44 | 8.86 | 3 |
| 5 | 5_1 | 92.29 | 15.87 | 3 |
| 13 | 13_1 | 57.43 | 3.65 | 3 |
| 14 | 14_1 | 94.93 | 19.91 | 3 |

A1CF Protein Level Quantification—Automated Western Blotting

A1CF levels in the RIPA protein lysates were quantified using the JESS system (proteinsimple) with the 12-230 kDa Jess or Wes Separation Module, 8×25 capillary cartridges (Protein Simple, #SM-W004) according to the manufacturer's instructions. The protein lysates were thawed and mixed well by pipetting, then 1.5 ul of 5× Fluorescent Sample buffer was mixed with 5 ul protein lysate. The samples were heated at 95° C. for 5 min and cooled to 4° C., then the samples were spun down for loading. Primary antibody mix by consisted of a 1:25 dilution of anti-A1CF (abcam, #ab231614) and 1:50 dilution of anti-ACTB (Sigma, #A1978-200 ul) antibodies in Milk-free Antibody Diluent (Protein Simple, #043-524). The secondary antibody mix consisted of a 1:20 dilution of 20× Anti-Mouse NIR antibody (Protein Simple, #043-821, 1:20) in Anti-Rabbit Secondary Antibody (Protein Simple, #042-206). Load and run using chemiluminescent and fluorescent readouts with 60 min for the primary antibody incubation time. The relative A1CF expression is calculated by normalizing the A1CF band to the ACTB band for each sample and then normalizing the treated to the non-treated control.

For all A1CF ASO treated samples, A1CF protein levels were under the limit of detection so the % inhibition could not be calculated.

HBsAg and HBeAg Levels in Supernatant

HBsAg and HBeAg levels in the supernatant of PHH cells were detected by diluting the supernatant 1:50 with 1×DPBS and then quantifying the antigens with chemiluminescence immunoassay (CLIA) kits (DiaSino® #DS1877032012V4, #DS1877012012V4) according to the manufacturer's instructions. Luminescence was detected on the Envision (Perkin Elmer) and Excel used to subtract background values and calculate percentage inhibitions based on the no drug control set to 100%.

The relative HBsAg and HBeAg expression levels in the supernatant of PHH cells are shown in Table 14 as % remaining of control, i.e. the lower the value the larger the inhibition.

TABLE 14
HBsAg and HBeAg levels in supernatant of PHH cells

| CNJ | HBsAg (%) | | | HBeAg (%) | | |
|---|---|---|---|---|---|---|
| ID NO | Mean | SD | N | Mean | SD | N |
| 6_1 | 37.71 | 4.30 | 3 | 35.29 | 2.63 | 3 |
| 1_1 | 7.57 | 2.07 | 3 | 5.21 | 3.52 | 3 |
| 7_1 | 0.46 | 0.16 | 3 | 2.56 | 2.44 | 3 |
| 7_2 | 0.45 | 0.49 | 3 | 4.45 | 4.71 | 3 |
| 8_1 | 0.46 | 0.89 | 3 | 0.60 | 2.84 | 3 |
| 2_2 | 69.79 | 2.50 | 3 | 52.43 | 2.37 | 3 |
| 2_1 | 28.10 | 13.29 | 3 | 14.40 | 7.73 | 3 |
| 3_1 | 17.22 | 14.53 | 3 | 7.17 | 4.42 | 3 |
| 9_1 | 108.38 | 3.92 | 3 | 92.16 | 4.13 | 3 |
| 10_1 | *BDL | 0.23 | 3 | 2.24 | 1.18 | 3 |
| 11_1 | 10.35 | 4.02 | 3 | 6.39 | 3.60 | 3 |
| 4_3 | 81.75 | 4.59 | 3 | 57.57 | 2.58 | 3 |
| 4_1 | 82.90 | 6.38 | 3 | 56.86 | 4.76 | 3 |
| 4_3 | 81.25 | 2.70 | 3 | 60.92 | 9.81 | 3 |
| 12_1 | 68.26 | 7.47 | 3 | 32.68 | 2.90 | 3 |
| 5_3 | 66.47 | 1.39 | 3 | 20.03 | 5.48 | 3 |
| 5_1 | 59.41 | 9.64 | 3 | 28.97 | 4.75 | 3 |
| 13_1 | 43.45 | 12.21 | 3 | 11.50 | 2.79 | 3 |
| 14_1 | 68.55 | 32.06 | 3 | 45.19 | 19.70 | 3 |

BDL = below detection limit

### Example 4—In Vitro Safety Assays

The safety of the ASOs was assessed using in vitro assays, namely a caspase activation assay, hepatotoxicity assay, renal toxicity assay and immunotoxicity assay. The results of these assays were expressed in a combined safety score.

Caspase Activation Assay

HepG2 cells were cultivated at app. 70% confluence in MEM medium with GlutaMax (Gibco #41090), supplemented with 10% heat inactivated fetal calf serum. Cells were detached with 0.25% Trypsin-EDTA solution (Gibco #25200056) and seeded into black, clear 96-well plates (Corning #3904, NY, USA) at a density of 1×10⁴ cells/well. 24 h post-seeding HepG2 cells were transiently transfected with Lipofectamine 2000 (Life Technologies #11668019) using 100 nM oligonucleotides dissolved in Opti-MEM (Gibco #31985). Caspase-3/7 activity was determined using the Caspase-Glo® 3/7 Assay (Promega Corporation, Madison WI, USA). Reconstituted Caspase-Glo® 3/7 reagent was added to the cells 24 hours post-transfection, incubated for 60 min, cell lysates were transferred into opaque 96-well plates (Corning #3600, NY, USA) before luminescence was determined on an Enspire multi-mode plate reader (Perkin Elmer) according to the manufacturer's instructions.

Data analysis: Each assay plate contains one toxic and two safe SSO controls; In parallel to calculating the data as % of the vehicle control [% V], we also calculate the actual assay window. This is based on the safe and the toxic control SSO, which allows determining the change of caspase3/7 proteases activities related to the assay window [% AW]

Hepatotoxicity Assay

Cryopreserved human hepatocytes (BioIVT-F00995, Lot QQE) or mouse hepatocytes (KalyCell) were thawed and seeded at a density of 25.000 cells/well for mouse hepatocytes and 40.000 cells/well for human hepatocytes on collagen-coated 96-well plate (Becton Dickinson AG, Allschwil, Switzerland). Cells were allowed to attach to the plates for 4 hours before ASO treatment.

Oligonucleotides were dissolved in PBS and 10× stock solutions are prepared in cell culture medium. ASOs were added to the cells with a maximal concentration of 30 μM for mouse hepatocytes and 100 μM for human hepatocytes without any assistance/transfection (gymnotic conditions). ASOs were left on the cells for 3 days, before cytotoxicity (LDH and ATP) assays were conducted [see References 1 and 2].

Renal Toxicity Assay

RPTEC/TERT1 cells were obtained from Evercyte GmbH, Vienna, Austria. The cells were routinely cultivated in DMEM/F12 medium supplemented with hormones, growth factors including 10 ng/ml recombinant human EGF as described in [2] plus addition of 2% FBS. Cells were seeded at a density of 18.000 cells/well in collagen-coated 96-well plates and cultivated for 3 days. On the day prior to starting treatment with oligonucleotides, the medium was changed to FBS-free medium and the cells were incubated for a further 24 hours. ASOs were dissolved in PBS and 10× stock solutions were prepared in FBS-free medium. ASOs were added to the cells at a final concentration of 100 M in triplicates. Treatment of the cells with compounds in fresh medium was repeated two times after 3 days and 6 days of incubation. Cell culture supernatants were harvested after 6 days of compound treatment for quantification of EGF consumption (i. e. EGF left over in medium). Intracellular ATP content was determined after a total of 9 days of compound treatment [see Reference 3].

Readouts for Caspase Activation, Hepatotoxicity and Renal Toxicity Assays

LDH assay—Cytotoxicity levels were determined by measuring the amount of LDH released into the culture media using a Cytotoxicity Detection Kit (Roche 11644793001, Roche Diagnostics GmbH Roche Applied Science Mannheim, Germany) and an EnSpire Multi Mode Plate Reader (Perkin Elmer Schweiz AG, Schwerzenbach, Switzerland) according to the manufacturer's protocol. Each sample was tested in triplicate.

Enzyme activity in the medium is expressed as percentage of total LDH activity in the supernatant of vehicle treated cells. Finally data are normalized to the assay window that is constituted by the negative and the positive control ASOon each plate to reveal the % assay window for the test item.

Intracellular ATP content—For the determination of cellular ATP levels the CellTiter-Glo® Luminescent Cell Viability Assay (G9242, Promega Corporation, Madison WI, USA) was used according to the manufacturer's protocol. Luminescence was recorded using an Envision plate reader (Perkin Elmer Schweiz AG, Schwerzenbach, Switzerland). ATP concentrations were calculated from a standard curve generated with ATP using the exact same reagents and treatment conditions. Each sample was tested in triplicate. Changes in ATP levels are calculated as % safe control or % assay window, which is generated by a safe and a toxic ASO control.

EGF Determination—For analysis of soluble EGF, frozen cell supernatants were thawed on ice, diluted 1:100 in PBS and analyzed by ELISA using Human EGF DuoSet ELISA (R&D #DY236-05) plus DuoSet Ancillary Reagent Kit 2 (R&D #DY008) according to the manufacturer's instructions. Data are reported as mean EGF concentrations and standard deviations of triplicate wells, and normalized on vehicle (PBS) control. Compounds are safe when normalized EGF value at 100 μM is below 5% of toxic control.

Immunotoxicity Assay [See Reference 4]

Venous blood from three separate healthy donors was collected in anticoagulant-sprayed vacutainer tubes (Sanquin Diagnostiek BV, Amsterdam NL) and kept at room temperature. Due to physico-chemical properties of the single stranded oligos (LNA) and for integrity of the Complement cascade, hirudin must be used as blood anticoagulant. Within 3 hour after collection, 195 μl of fresh whole blood was added in triplicates to U-bottom wells of 96-well plates containing 5 μl of the items to be tested. Test items were analyzed at final concentrations 50 μM. Blood cells responsiveness to innate immune stimuli and endogenous activation level were assessed by including controls containing PBS, stabilizing solution, and TLR activators (R848, CpG and polyDC, InvivoGen). Internal controls for complement stimulation were also included (Complement activator, TECO medical and Zymosan, Sigma).

After incubation at 37° C. with 5% CO2, cells and plasma were separated by centrifugation at 1800 g for 5 min. Plasma samples for Complement analysis were collected after 45 min, diluted 1:1 in stabilizing solution and frozen in aliquots at −70° C. until analysis. Analyte concentrations were determined by individual sandwich ELISA using the different human assay kits according to the manufacturer's instructions (Human Complement Plus EIA kits: C3a Cat No. A015, C5a Cat No. A025, from Quidel TECOmedical AG, Sissach, Switzerland). Plates were analyzed on Versamax microplate ELISA reader (Bucher Biotec, Basel, Switzerland) using the Softmax Pro software v.5.2.

Determination of cytokine concentrations in frozen plasma samples collected at 6 hours, was performed by multiplex ELISA using the Human Soluble Protein Flex Assay (BD CBA Human Soluble Protein assay) with the FCAP Array analysis software.

Triplicate values were averaged for each concentration and stimulation index (SI, relative to negative control reference ASO) was calculated for each donor. Mean stimulation index for the 3 donors was used to rank the immunotoxicity potential of the test items: ASO with a SI lower than 2 are considered of low immunotoxicity potential. Test items ASO having a SI between 2 and 5 are concluded to be of medium immunotoxicity risk, while a SI greater the 5 qualifies the ASO as of high immunotoxicity potential.

Final Safety Score Calculation

The criterion for selection of oligonucleotides assessed in the various safety assays is based on the magnitude and frequency of signals obtained. The signals obtained in the individual in vitro safety assays result in a score (0=safe, 0.5=borderline toxicity, 1=mild toxicity, 2=medium toxicity and 3=severe toxicity) and are summarized into a cumulative score for each sequence, that allows clear ranking of compounds. The signal strength is a measure of risk for in vivo toxicity based on validation of the assays using in vivo relevant reference molecules

### REFERENCES

1. Sewing S. et al. (2016) Establishment of a Predictive In Vitro Assay for Assessment of the Hepatotoxic Potential of Oligonucleotide Drugs. PLoS ONE 11(7): e0159431
2. WO2017/067970 A1 IN VITRO TOXICITY SCREENING ASSAY PCT/EP2016/075060 Sabine Sewing, Adrian B. Roth, Annie Moisan, Cristina Bertinetti-Lapatki, Franziska Boess
3. Moisan A. et al. (2017) Inhibition of EGF Uptake by Nephrotoxic Antisense Drugs In Vitro and Implications for Preclinical Safety Profiling. Molecular Therapy: Nucleic Acids 6, 89-105
4. Sewing S. et al. (2018) Assessing single-stranded oligonucleotide drug-induced effects in vitro reveals key risk factors for thrombocytopenia. PLoS ONE 12(11): e0187574

TABLE 15
In vitro safety results

| | | Caspase | | | | | | | | | | | | | |
|---|---|---|---|---|---|---|---|---|---|---|---|---|---|---|---|
| Antisense | | activation | | Hepatotoxicity | | | | | | Immunotoxicity | | Renal toxicity | | | |
| oligonucleotide | | HepG2 | | mouse 30 μM | | | human 100 μM | | | 50 μM | | Human PTEC 100 μM | | | |
| CMP ID | CNJ ID | 100 nM | | LDH | ATP | | LDH | ATP | | Complement | Cytokines | ATP | EGF | | Total |
| NO | NO | % AW | Score | % AW | % AW | Score | % AW | % AW | Score | Score | Score | % AW | % AW | Score | score |
| 1_1 | 1_1 | 5 | 0 | 39 | 0 | 1 | 17 | 5 | 0 | 0 | 0 | 95 | −1 | 0 | 1 |
| 7_2 | 7_2 | 6 | 0 | 25 | −5 | 0.5 | 15 | 15 | 0 | 0 | 1 | 91 | 1 | 0 | 1.5 |
| 2_1 | 2_1 | 10 | 0 | 24 | 10 | 0.5 | 6 | 17 | 0 | 0 | 1 | 107 | 0 | 0 | 1.5 |
| 4_3 | 4_3 | 12 | 0 | 17 | 1 | 0 | 11 | 29 | 1 | 0 | 1 | 108 | 2 | 0 | 2 |
| 5_1 | 5_1 | 4 | 0 | 21 | −11 | 0.5 | 24 | 6 | 0.5 | 0 | 1 | 111 | 2 | 0 | 2 |
| 4_1 | 4_1 | −1 | 0 | 20 | −2 | 0 | 10 | 30 | 1 | 0 | 1 | 87 | 3 | 0 | 2 |
| 13_1 | 13_1 | −7 | 0 | 14 | 2 | 0 | 8 | 9 | 0 | 0 | 2 | 94 | 3 | 0 | 2 |
| 3_1 | 3_1 | 9 | 0 | 33 | 8 | 1 | 24 | 15 | 0.5 | 0 | 0 | 97 | 12 | 1 | 2.5 |
| 4_2 | 4_2 | −1 | 0 | 22 | −2 | 0.5 | 12 | 27 | 1 | 0 | 1 | 99 | 0 | 0 | 2.5 |
| 6_1 | 6_1 | −4 | 0 | 32 | −5 | 1 | 29 | 29 | 1 | 0 | 1 | 92 | −2 | 0 | 3 |
| 10_1 | 10_1 | 1 | 0 | 0 | 28 | 1 | 7 | 19 | 0 | 0 | 0 | 73 | 23 | 2 | 3 |
| 14_1 | 14_1 | 14 | 0 | 20 | 7 | 0 | 18 | 20 | 0.5 | 0 | 2 | 110 | 7 | 0.5 | 3 |
| 7_2 | 7_2 | 22 | 0.5 | 28 | 7 | 1 | 23 | 23 | 0.5 | 0 | 1 | 93 | 9 | 0.5 | 3.5 |
| 12_1 | 12_1 | 7 | 0 | 21 | −6 | 0.5 | 24 | 28 | 1 | 0 | 2 | 107 | 1 | 0 | 3.5 |
| 5_3 | 5_3 | −7 | 0 | 33 | −2 | 1 | 5 | 22 | 0.5 | 1 | 1 | 97 | 3 | 0 | 3.5 |
| 8_1 | 8_1 | −1 | 0 | 27 | 1 | 1 | 11 | −2 | 0 | 1 | 2 | 103 | −1 | 0 | 4 |
| 11_1 | 11_1 | 12 | 0 | 3 | 9 | 0 | 8 | 37 | 1 | 1 | 2 | 101 | 10 | 1 | 5 |
| 2_2 | 2_2 | 9 | 0 | 24 | 5 | 0.5 | 0 | 13 | 0 | 2 | 2 | 97 | 5 | 0.5 | 5 |
| 9_1 | 9_1 | −6 | 0 | 15 | 8 | 0 | 22 | 39 | 1 | 2 | 2 | 101 | 11 | 1 | 6 |
| 5_2 | 5_2 | 8 | 0 | 21 | −5 | 0.5 | 14 | 8 | 0 | 0 | 0 | 110 | 2 | 0 | 0.5 |
| 15_1 | 15_1 | 10 | 0 | 9 | −8 | 0 | 15 | 56 | 2 | ND | ND | 111 | 6 | 0.5 | 2.5 |
| 16_1 | 16_1 | 14 | 0 | 20 | −5 | 0.5 | 16 | 39 | 1 | ND | ND | 107 | 6 | 0.5 | 2.0 |
| 16_2 | 16_2 | 11 | 0 | 14 | 11 | 0 | 9 | 32 | 1 | 0 | 0 | 108 | 5 | 0.5 | 1.5 |
| 16_3 | 16_3 | 10 | 0 | 5 | 3 | 0 | 10 | 38 | 1 | ND | ND | 108 | 3 | 0 | 1.0 |
| 2_3 | 2_3 | 7 | 0 | 11 | −2 | 0 | 14 | 38 | 1 | ND | ND | 106 | 5 | 0 | 1.0 |
| 2_4 | 2_4 | 4 | 0 | 12 | −1 | 0 | 16 | 48 | 2 | ND | ND | 105 | 4 | 0 | 2.0 |
| 2_5 | 2_5 | 8 | 0 | 20 | 8 | 0.5 | 11 | 39 | 1 | ND | ND | 106 | 6 | 0.5 | 2.0 |
| 2_6 | 2_6 | −6 | 0 | 8 | 7 | 0 | 5 | 47 | 2 | ND | ND | 109 | 5 | 0 | 2.0 |
| 2_7 | 2_7 | 10 | 0 | 20 | −12 | 0.5 | 21 | 40 | 1 | ND | ND | 116 | 2 | 0 | 1.5 |
| 2_8 | 2_8 | 7 | 0 | 21 | −13 | 0.5 | 15 | 38 | 1 | ND | ND | 117 | 3 | 0 | 1.5 |
| 2_9 | 2_9 | 18 | 0 | 22 | 2 | 0.5 | 17 | 24 | 0.5 | 0 | 0 | 101 | 3 | 0 | 1.0 |
| 2_10 | 2_10 | 17 | 0 | 23 | 7 | 0.5 | 12 | 30 | 1 | ND | ND | 106 | 4 | 0 | 1.5 |
| 2_11 | 2_11 | 13 | 0 | 22 | −11 | 0.5 | 27 | 38 | 1 | ND | ND | 109 | 2 | 0 | 1.5 |
| 2_12 | 2_12 | 19 | 0 | 25 | −5 | 1 | 11 | 33 | 1 | ND | ND | 115 | 3 | 0 | 2.0 |
| 17_1 | 17_1 | 7 | 0 | 16 | 4 | 0 | 16 | 26 | 1 | 0 | 0 | 109 | 2 | 0 | 1.0 |
| 18_1 | 18_1 | 14 | 0 | 23 | 6 | 0.5 | 12 | 21 | 0.5 | 0 | 0 | 114 | 3 | 0 | 1.0 |
| 19_1 | 19_1 | 11 | 0 | 16 | −12 | 0 | 12 | 32 | 1 | 0 | 0 | 113 | 3 | 0 | 1.0 |
| 20_1 | 20_1 | −7 | 0 | 22 | −7 | 0.5 | 6 | 42 | 2 | ND | ND | 101 | 4 | 0 | 2.5 |
| 20_2 | 20_2 | −8 | 0 | 20 | −14 | 0 | −2 | 38 | 1 | ND | ND | 102 | 3 | 0 | 1.0 |
| 20_3 | 20_3 | −7 | 0 | 18 | −5 | 0 | −3 | 26 | 1 | 1 | 1 | 100 | 2 | 0 | 3.0 |
| 20_4 | 20_4 | −4 | 0 | 0 | −7 | 0 | 0 | 33 | 1 | 1 | 0 | 107 | 2 | 0 | 2.0 |
| 20_5 | 20_5 | −8 | 0 | 17 | −9 | 0 | −3 | 41 | 2 | ND | ND | 102 | 2 | 0 | 2.0 |
| 20_6 | 20_6 | −4 | 0 | 16 | −5 | 0 | 5 | 40 | 1 | ND | ND | 104 | 2 | 0 | 1.0 |
| 6_2 | 6_2 | −6 | 0 | 18 | −6 | 0 | 3 | 52 | 2 | ND | ND | 105 | 4 | 0 | 2.0 |
| 6_3 | 6_3 | −3 | 0 | 8 | −7 | 0 | 7 | −26 | 0 | 0 | 0 | 115 | 2 | 0 | 0.0 |
| 6_4 | 6_4 | −1 | 0 | 22 | 12 | 0.5 | 5 | 41 | 2 | ND | ND | 108 | 4 | 0 | 2.5 |
| 6_5 | 6_5 | −3 | 0 | ND | ND | ND | 9 | 40 | 1 | ND | ND | 92 | 4 | 0 | ND |
| 6_6 | 6_6 | −4 | 0 | −1 | 13 | 1 | 9 | 45 | 2 | ND | ND | 95 | 7 | 0.5 | 2.5 |
| 6_7 | 6_7 | 5 | 0 | 33 | 14 | 1 | 7 | 40 | 2 | ND | ND | 103 | 3 | 0 | 3.0 |
| 6_8 | 6_8 | −3 | 0 | 15 | 6 | 0 | 5 | 32 | 1 | 0 | 1 | 108 | 2 | 0 | 2.0 |
| 6_9 | 6_9 | −5 | 0 | ND | ND | ND | ND | ND | ND | ND | ND | ND | ND | 0 | ND |
| 6_10 | 6_10 | −3 | 0 | 15 | 9 | 0 | 15 | 43 | 2 | ND | ND | 104 | 3 | 0 | 2.0 |
| 6_11 | 6_11 | −2 | 0 | ND | ND | ND | ND | ND | ND | ND | ND | ND | ND | 0 | ND |
| 6_12 | 6_12 | −5 | 0 | 8 | 16 | 0 | 19 | 51 | 2 | ND | ND | 109 | 3 | 0 | 2.0 |
| 21_1 | 21_1 | −7 | 0 | 17 | −16 | 0 | −6 | −19 | 0 | 0 | 1 | 103 | 2 | 0 | 2.0 |
| 22_1 | 22_1 | −6 | 0 | 14 | −8 | 0 | −2 | −4 | 0 | 0 | 0 | 102 | 2 | 0 | 0.0 |
| 23_1 | 23_1 | −6 | 0 | 19 | −10 | 0 | −11 | 13 | 0 | 0 | 0 | 102 | 2 | 0 | 0.0 |
| 24_1 | 24_1 | 4 | 0 | 22 | −19 | 0.5 | −11 | 12 | 0 | 0 | 0 | 87 | 6 | 0.5 | 1.0 |
| 25_1 | 25_1 | −7 | 0 | 9 | −16 | 0 | −10 | 7 | 0 | 0 | 0 | 95 | 4 | 0 | 0.0 |
| 25_2 | 25_2 | −3 | 0 | 24 | −23 | 0.5 | −16 | 7 | 0 | 0 | 0 | 86 | 5 | 0.5 | 1.0 |
| 26_1 | 26_1 | −3 | 0 | 27 | −16 | 1 | −13 | 10 | 0 | 0 | 1 | 92 | 5 | 0 | 2.0 |
| 27_1 | 27_1 | −5 | 0 | 23 | −12 | 0.5 | −9 | 31 | 1 | ND | ND | 110 | 3 | 0 | 1.5 |
| 27_2 | 27_2 | −5 | 0 | 28 | −9 | 1 | 7 | 42 | 2 | ND | ND | 102 | 2 | 0 | 3.0 |
| 28_1 | 28_1 | 2 | 0 | 28 | −2 | 1 | 4 | 40 | 2 | ND | ND | 100 | 3 | 0 | 3.0 |
| 29_1 | 29_1 | −5 | 0 | 19 | −1 | 0 | 4 | 39 | 1 | ND | ND | 94 | 5 | 0 | 1.0 |
| 30_1 | 30_1 | −4 | 0 | 19 | 2 | 0 | 5 | 30 | 1 | 0 | 0 | 100 | 4 | 0 | 1.0 |
| 11_2 | 11_2 | 19 | 0 | 13 | −3 | 0 | 14 | 44 | 2 | ND | ND | 101 | 3 | 0 | 2.0 |
| 11_3 | 11_3 | 24 | 0.5 | 20 | −3 | 0 | 15 | 35 | 1 | ND | ND | 100 | 3 | 0 | 1.0 |

ND = not determined

### Example 5—4-Week Subcutaneous Exploratory Toxicity Study in Rats (In Vivo Safety)

Study Design

The study was conducted as an initial screen for major target organ toxicity and to rank the oligonucleotides regarding potential safety liabilities. In the study, four male rats (Wistar Han IGS Crl:WI, approximately 8 weeks old at study start) were administered subcutaneously once weekly on days 1, 8, 15 and 22 at 0 (vehicle control), 15 or 45 mg/kg/administration of each oligonucleotide (CNJ ID NO 1_1, CNJ ID NO 2_1, CNJ ID NO 3_1, CNJ ID NO 4_2 and CNJ ID NO 5_1). Three days after the last dose (day 25) following overnight food deprivation, animals were anesthetized by intraperitoneal injection of 150 mg/kg pentobarbital followed by decapitation or bilateral pneumothorax. Organs and tissues from all animals were examined in situ, removed, and checked for abnormalities. Serial sections were prepared from formalin-fixed, paraffin-embedded organs, i.e. liver, kidney, spleen, axillary lymph node, lung, heart, ileum, stomach and injection site. The sections were stained with hematoxylin-eosin. Assessment of toxicity was based upon mortality, in-life observations, body weight, food consumption, spleen weights, clinical and anatomic pathology.

Results

All oligonucleotides tested in the rat study reduced body weight gain and food consumption. Test item-related histopathological findings indicative of an immunostimulatory effect were observed with all oligonucleotides in liver, kidney, spleen, axillary lymph node, lung and injection site. Histopathological changes most probably secondary to oligonucleotide accumulation were observed mainly in the kidney and liver. Increased liver enzymes were observed with CNJ ID NO 1_1, CNJ ID NO 4_2 and CNJ ID NO 5_1 in correlation with hepatocellular single cell necrosis at the low and high dose level. Treatment with CNJ ID NO 3_1 resulted in the same findings, but limited to the high dose level in ¼ animals. Adverse histopathological findings in the kidney, i.e. tubular degeneration/regeneration were observed only for CNJ ID NO 4_2 and CNJ ID NO 5_1 at the high dose. No test item-related findings were observed in the heart, ileum and stomach with any compound.

CNJ ID NO 2_1 was tolerated well and did not result in any adverse effects, except non-adverse immunostimulatory changes.

### Example 6—84-Day Anti-Viral Efficacy Study in HBV-Infected Mice

This example describes testing of anti-viral efficacy of select oligonucleotides in vivo using HBV-infected mice with a humanized liver (PhoenixBio, PXB-mouse).

Materials & Methods

Compounds Formulation

All compounds were formulated in sterile saline.

Mice

The use of the animals for this study was approved by the Animal Ethics Committee of PhoenixBio (Resolution No: 2743). Mouse study PBCA-00130 included seventy PXB-mice® (PhoenixBio Co.; Ltd.) with a humanized liver [Uchida et al. Usefulness of humanized cDNA-uPA/SCID mice for the study of hepatitis B virus and hepatitis C virus virology. J Gen Virol. 2017 May; 98(5):1040-1047] that were infected with HBV genotype C as previously described for 69 days prior to the first dosing. On the day preceding the start of compound administration, all the candidate animals were weighed and those with a healthy appearance and which met all of the criteria regarding the age, weight, serum viremia were assigned to the study. To minimize variance between the groups, the group composition was randomized based on the arithmetic mean values for body weight and blood h-Alb concentration and geometric mean values for serum HBV DNA concentration.

The mice were dosed with a subcutaneous injection of the dose formulation based on the dosing schedule below and sacrificed on day 84 post-dosing. Each group consisted of 8 PXB mice infected with HBV.

Dosing Schedule:

| | | Dose | | | |
|---|---|---|---|---|---|
| | Test | Level | Conc. | Volume | |
| Group | compounds | (mg/kg) | (mg/mL) | (mL/kg) | Frequency |
| 1 | Vehicle | 0 | 0 | 10 | Weekly, 12 times, |
| | | | | | days 0, 7, 14, 21, |
| | | | | | 28, 35, 42, 49, |
| | | | | | 56, 63, 70, 77 |
| 2 | CNJ ID | 10 | 1 | 10 | Weekly, 9 times, |
| | NO 2_1 | | | | days 0, 7, 14, 21, |
| | | | | | 28, 35, 42, 49, 56 |
| 3 | CNJ ID | 10 | 1 | 10 | Weekly, 12 times, |
| | NO 3_1 | | | | days 0, 7, 14, 21, |
| | | | | | 28, 35, 42, 49, |
| | | | | | 56, 63, 70, 77 |

Blood Collection and Serum Separation

For the serum preparations 75 μl blood was drawn per time point per animal. Two microliters of the blood were used for blood human albumin quantification. The remaining 73 μl were left at room temperature for at least 5 minutes to coagulate and then centrifuged at 13200×g, 4° C. for 3 minutes to obtain serum.

Terminal Sacrifice

At sacrifice, day 84 post-dosing, the mice were anaesthetized with isoflurane anesthesia and a minimum of 300 μL of blood was collected from each animal via the heart into syringes after which the animals were sacrificed by cardiac puncture and exsanguination. Necropsy was performed after the whole blood was collected at sacrifice. Whole livers from all animals were harvested and blot dried. The gallbladder was removed and several small liver tissue sections cut and snap-frozen directly for the intrahepatic readouts.

Liver Hirt Extraction

Small pieces of liver tissue were homogenized with 1 ml of cccDNA extraction buffer (50 mM Tris-HCl, pH 7.5; 150 mM NaCl; 10 mM EDTA; 1% SDS) for 20 secs at 6000 rpm at RT in MagNA Lyser Green Bead tubes (Roche, 03358941001) using the FastPrep-24 5G Sample preparation System (M.P. Biomedicals). Samples were spun down, and the lysate transferred to a fresh tube with 250 μl 5M NaCl (Gibco, #24740-011). The samples were rotated at 4° C. overnight, then spun down and the supernatant transferred to a new tube. The DNA was extracted thrice with 1 mL UltraPure Buffer-Saturated Phenol (Life Technologies, #15513-039), then once with 1 mL UltraPure Phenol:Chloroform:Isoamyl Alcohol (25:24:1) (Life Technologies, #15593-031). The aqueous phase was then mixed with 2.2 volumes of EtOH and precipitated at −80° C. overnight. The DNA was pelleted and washed with 70% EtOH thrice, then air dried and resuspended in 50 μl 10 mM Tris-HCl, pH8.

Human Beta Globin (HBB) qPCR

A 1:50 dilution of the Hirt extracted DNA is then used to determine the HBB copy number by qPCR. For this purpose 5 ul TaqMan Fast Advanced Master Mix (Applied Biosystems, #4444557), 0.5 uL 20× Taqman Assay (HBB Hs00758889_s1, VIC), 0.5 uL H2O and 4 μl diluted DNA were mixed per reaction. A genomic DNA standard was also prepared and included in the qPCR to allow copy number calculations. The QuantStudio 12KFlex Cycler was run at 95° C. for 20 seconds, then 40 cycles with 95° C. for 1 second and 60° C. for 20 seconds).

Southern Blot

Southern blotting was done by loading a defined amount of HBB copies per sample (525′000 copies) onto a 0.95% agarose gel and running for 3.5 h at 50V. After this, the gel was incubated in 0.2M HCl for 10 mins at RT, denaturing buffer (0.5 M NaOH, 1.5 M NaCl) for 30 mins at RT, neutralizing buffer (0.5 M Tris-HCl pH 7.5, 1.5 M NaCl) for 30 mins at RT and finally in UltraPure 20×SSC buffer (Life Technologies, #15557-036) for 30 mins at RT. The transfer was to Hybond-XL membrane (GE Healthcare, #RPN2020S) was done with the Whatman Nytran SuPerCharge (SPC) TurboBlotter Kit (Sigma, #WHA10416328) overnight. The membrane was UV crosslinked at 1800×100 μJ/cm2 once and dried. Then the membrane was pre-hybridized at 37° C. for 1 h in DIG Easy Hyb buffer (Roche, #11603558001). A DIG labeled HBV specific probe was prepared using the PCR DIG Probe Synthesis Kit (Merk, 11636090910) with a HBV specific plasmid as template, heated to 100° C. for 5 minutes and then rapidly cooled down and incubated on the membrane in fresh 37° C. warm hybridization buffer overnight. The membrane was washed with SSC wash buffer I (2×SSC, 0.1% SDS) 2×5 min at RT and SSC wash buffer II (0.5×SSC, 0.1% SDS) 2×15 min at 65° C. The DIG Wash and Block buffer Set (Roche, #11585762001) was then used according to the manufacturer's instructions and luminescence intensity detected with CDP-Star with NitroBlock solution in a Fusion Fx (VILBER). Mice dead prior to the sacrifice at day 84 were excluded from the analysis. Two vehicle mice with mouse lymphoma were excluded from analysis as too low number of HBB copies loaded. Luminescence intensity of the cccDNA bands was quantified using the Image Studio Lite version 5.2 software (LI-COR Biosciences). The remaining percentage of cccDNA was calculated as follows for each treatment group compared to the vehicle group:

% cccDNA remaining=(average of intensities for the treated mice*100)/average of intensities for the vehicle mice.

Measuring Serum HBsAg Concentration

Serum HBsAg concentration was determined by SRL, Inc. (Tokyo, Japan) based on Chemiluminescent Enzyme Immuno Assay (CLEIA) developed by Fujirebio (LUMIPULSE HBsAg-HQ, LUMIPULSE® Presto II). The dilution factor was 60, and the measurement range of this assay was between 0.005 and 150 IU/mL. Mice dead prior to the sacrifice at day 84 were excluded from the analysis. Mice with serum HBsAg levels that were outliers from the group at day 0 were excluded from the analysis.

Measuring Serum HBeAg Concentration

Serum HBeAg concentration was determined by SRL, Inc. based on Chemiluminescent Enzyme Immuno Assay (CLEIA) developed by Fujirebio (LUMIPULSE HBeAg, LUMIPULSE® Presto II). The dilution factor was 60, and the measurement range of this assay was between 0.1 and 1590 C.O.I. Mice dead prior to the sacrifice at day 84 were excluded from the analysis.

Mice with serum HBeAg levels that were outliers from the group at day 0 were excluded from the analysis.

Measuring Serum HBcrAg Concentration

Serum HBcrAg concentration was determined by SRL, Inc. using ChemiLuminescence Enzyme ImmunoAssay (CLEIA) developed by Fujirebio Inc. (LUMIPULSE HBcrAg, LUMIPULSE F). The lowest quantification limit of this assay was 3.0 log U/mL. In this study, the dilution factor was 300. Mice dead prior to the sacrifice at day 84 were excluded from the analysis.

Measuring Circulating HBV RNA in Serum

Serum was diluted 1:12.5 and HBV RNA levels were detected with the quantitative Cobas® HBV RNA investigational test that is run on the Cobas® 6800/8800 System according to the manufacturer's instructions. The viral load (copies/mL) is quantified against a non-HBV RNA quantitation standard (RNA-QS), which is introduced into each specimen during sample processing. In addition, the test utilizes three external controls: a high titer positive, a low titer positive, and a negative control. Mice dead prior to the sacrifice at day 84 were excluded from the analysis.

Measuring Human Albumin (hAlb) in Blood

Two microliters of blood were diluted in saline. The clinical chemistry analyzer BioMajesty™ Series JCA-BM6050 (JEOL Ltd., Tokyo, Japan) was used to measure the blood h-Alb concentration in mg/mL using latex agglutination immunonephelometry LZ Test “Eiken” U-ALB (Eiken Chemical Co., Ltd., Tokyo, Japan). Mice dead prior to the sacrifice at day 84 were excluded from the analysis.

Measuring Intrahepatic pgRNA, Intrahepatic A1CF mRNA and Intrahepatic HBV RNA

Sample Handling

Tissue sections were lysed and homogenized in 1400 μl MagNaPure LC RNA Isolation Tissue buffer (Roche, #03604721001) using 2 mL MagNA Lyser Green Beads (Roche, #03358941001) at 6000 rpm for 20 secs. The homogenates were then incubated at RT for 30 mins and the insoluble debris removed by centrifugation.

RNA Isolation and Quantification of A1CF, pgRNA and HBV RNA Transcripts by qPCR

Total RNA was extracted from the tissue homogenates using a MagNA Pure robot and the MagNA Pure 96 Cellular RNA Large Volume Kit (Roche, #05467535001) according to the manufacturer's protocol using the “Cellular RNA LV” protocol. 350 μl of tissue homogenate was loaded in the robot and the final elution volume was 50 ul.

RNA concentration and quality was checked on the isolated RNA samples (by NanoDrop), and they were all normalized to a concentration of 7.5 ng/ul in a dilution plate (Thermo scientific—#AB0900). The RNA was mixed, the plate sealed and the RNA heat shocked for 40 seconds at 90 C to melt RNA:LNA duplexes and then put on ice. A standard curve was made from saline samples as a 8-point 2-fold dilution series of the RNA.

The one-step qPCR mix was prepared and pipetted into a 384-well qPCR plate (MicroAmp Optical 384-well plate—Applied Biosystems 4309849). Per reaction (10 μl), 5 μL qScript™ XLT One-Step RT-qPCR ToughMix®, Low ROX™ (Quanta Bioscience cat 95134-500), 0.25 uL assay mix (see below) and 0.75 uL water was mixed. 6 uL of this mix was added to the 384 well plate and 4 μL diluted RNA added on top. After a quick spin, the following qPCR program was run on a ViA 7 (Thermo): 15 minutes at 50 C, then 3:30 minutes at 95 C, followed by 40 cycles of 95° C. for 5 secs and 60° C. for 45 secs.

| | Vendor |
|---|---|
| Human A1CF | |
| A1CF_Hs00205840_m1 | Thermo |
| Human Housekeeping genes | |
| GAPDH_Hs.PT.39a.22214836 | IDT |
| TBP_Hs.PT.58v39858774 | IDT |
| GUSB_Hs.PT.58v27737538 | IDT |
| PGK1_Taqman_Hs9999906_m1 | Thermo |
| HPRT1_Hs.PT.58v45621572 | IDT |
| Viral readouts | |
| pgRNA_Taqman_pgRNA_AILJKX5 | Thermo |
| HBV-RNA_taqman_Pa03453406_s1 | Thermo |

The qPCR results were analyzed in QuantStudio. The expression of each transcript was calculated using the standard curve. Human A1CF expression was normalized to the geometric mean of the four housekeeping genes shown above. The results are shown as percent of the average of the saline group.

Measuring Oligonucleotide Exposure by Hybridization ELISA

Oligonucleotide exposure (i.e. the amount of molecule present in a tissue) was measured using a two-probe hybridization ELISA assay system. In short, the ASO standard (unconjugated molecules) or tissue sample homogenate (aliquot of the samples used for qPCR above) was diluted in a buffer containing a biotin-conjugated capture probe and a digoxigenin-conjugated detection probe, bound to a streptavidin-coated assay plate and quantified through use of a digoxigenin-alkaline phosphatase secondary antibody and absorbance readout.

The protocol used was as follows. Samples were diluted in a 5×SSCT buffer (750 mM NaCl, and 75 mM sodium citrate, containing 0.05% (v/v) Tween-20). Appropriate standards matching sample matrix and dilution factor were run on every plate.

Sample and standards were added to a dilution plate (for CNJ ID NO 2_1: Polypropylene 96-well plate with round bottom, Thermo—for CNJ ID NO 3_1: Eppendorf twin.tec PCR plate 96 LoBind, semi-skirted—cat no: 0030129504) in the desired setup and dilution series were made. 250 μL sample/standard plus capture-detection solution was added to the first wells and 125 μL capture-detection solution in the remaining wells. The capture-detection solutions used were:

| | | |
|---|---|---|
| Oligonucleotide | 5′-[5gn2c6]- | 5′-[5gn2c6]- |
| (conjugated) | CAAAAAGTTTCTCAGATAGGC-3′ | CAGGACATGTTAATTTTACTT-3′ |
| | (CNJ ID NO 2_1) | (CNJ ID NO 3_1) |
| Compound | 5′-CAAAAAGTTTCTCAGATAGGC-3′ | 5′-CAGGACATGTTAATTTTACTT-3′ |
| (unconjugated) | (CMP ID NO 2_1) | (CMP ID NO 3_1) |
| Capture probe | 5′-BIO-GCCTATCTGAG-3′ | 5′-BIO-AAGTAAAAT-3′ |
| | (SEQ ID NO 60) | |
| Detection probe | 5′-AACTTTT-DIG-3′ | 5′-ATGTCC-DIG-3′ |
| Probe | 10 | 100 |
| concentration | | |
| in 5x SSCT | | |
| buffer (nM) | | |

A two-fold dilution series of standards (of the unconjugated analyte) and samples was made by transferring 125 μL liquid sequentially. 2-4 wells were kept for blanks (capture-detection solution only). A two-fold sample dilution series of at least 6 wells is recommended for optimal results.

Dilution plates for CNJ ID NO 2_1 quantification were placed for 30 minutes at RT.

Dilution plates for CNJ ID NO 31 quantification were incubated for 5 minutes at 90° C. (using a thermocycler) and then left for 30 minutes at RT.

For both oligonucleotides, the procedure was then:

100 μL of liquid was transferred from the dilution plate to a streptavidin plate (Roche Cat. No. 11989685001). The plate was incubated for 1 hour at RT with gentle agitation (plate shaker).

The wells were aspirated and washed three times with 300 μL of 2×SSCT buffer (300 mM NaCl, and 30 mM sodium citrate, containing 0.05% (v/v) Tween-20).

100 μL anti-DIG-AP (Roche Applied Science, Cat. No. 11 093 274 910) diluted 1:4000 in PBST (0.05% tween-20 added) (made on the same day) was added to each well and incubated for 1 hour at room temperature under gentle agitation.

The wells were aspirated and washed three times with 300 μL of 2×SSCT buffer.

100 μL of substrate (AP) solution (freshly prepared, Blue Phos Substrate, KPL product code 50-88-00) was added to each well. Protective eyewear was worn when handling AP substrate A.

The intensity of the colour was measured spectrophotometrically at 615 nm after a 30-minute incubation with gentle agitation. For the manual ELISA procedure plates were transferred directly to the reader and read every 5 min from t=0 to t=45, with shaking before each reading.

Raw data were exported from the readers (Gen5 2.0 software) to excel format and further analyzed in excel. Standard curves were generated using GraphPad Prism 6 software and a logistic 4PL regression model (log concentration vs absorbance). Data points are reported as the mean value of the technical replicates coming from all the wells accepted in the analysis (above 5× background signal and below signal saturation level).

Results

cccDNA Expression Level by Southern Blot in the Liver of PXB Mice at Day 84

Remaining cccDNA is shown in Table 16 as % of vehicle mice. cccDNA band intensity was measured and normalized to the vehicle mice.

TABLE 16
cccDNA level in the liver of PXB mice

| Group | N | Mean of remaining % cccDNA | SEM |
|---|---|---|---|
| Vehicle | 3 | 100.00 | 23.37 |
| CNJ ID NO 2_1 | 3 | 64.88 | 16.80 |
| CNJ ID NO 3_1 | 5 | 64.48 | 13.97 |

HBsAg in Serum of PXB Mice

HBsAg level at day 0 and day 84 is shown in Table 17 as % of day 0 as baseline.

TABLE 17
Relative HBsAg in the serum of PXB mice

| | % of remaining serum HBsAg | | | | | | | | |
|---|---|---|---|---|---|---|---|---|---|
| | | | | CNJ ID NO | | | CNJ ID NO | | |
| study | Saline | | | 2_1 | | | 3_1 | | |
| day | Mean | SEM | N | Mean | SEM | N | Mean | SEM | N |
| 0 | 100 | 0 | 4 | 100 | 0 | 3 | 100 | 0 | 4 |
| 84 | 136.59 | 19.79 | 4 | 88.26 | 1.37 | 3 | 71.38 | 8.92 | 4 |

HBeAg in Serum of PXB Mice

HBeAg level at day 0 and day 84 is shown in Table 18 as % of day 0 as baseline.

TABLE 18
Relative HBeAg in the serum of PXB mice

| | % of remaining serum HBeAg | | | | | | | | |
|---|---|---|---|---|---|---|---|---|---|
| | | | | CNJ ID NO | | | CNJ ID NO | | |
| study | Saline | | | 2_1 | | | 3_1 | | |
| day | Mean | SEM | N | Mean | SEM | N | Mean | SEM | N |
| 0 | 100 | 0 | 5 | 100 | 0 | 3 | 100 | 0 | 4 |
| 84 | 109.32 | 12.33 | 5 | 63.10 | 10.48 | 3 | 44.10 | 6.15 | 4 |

HBcrAg in Serum at Day 84

HBcrAg level at day 84 is shown in Table 19 as log U/mL.

TABLE 19
HBcrAg in the serum of PXB mice

| Group | N | Mean of HBcrAg in LogU/mL | SEM |
|---|---|---|---|
| Vehicle | 5 | 8.3 | 0.04 |
| CNJ ID NO 2_1 | 3 | 8.067 | 0.03 |
| CNJ ID NO 3_1 | 5 | 7.96 | 0.05 |

Circulating HBV RNA in the Serum at Day 84

Remaining circulating HBV RNA at day 84 is shown in Table 20 as % of remaining vs vehicle.

TABLE 20
Relative circulating HBV RNA level in the serum of PXB mice

| Group | N | % of remaining circHBV RNA | SEM |
|---|---|---|---|
| Vehicle | 5 | 100 | 33.69 |
| CNJ ID NO 2_1 | 3 | 38.86 | 14.66 |
| CNJ ID NO 3_1 | 5 | 61.55 | 16.58 |

pgRNA Level at Day 84

Relative pgRNA level at day 84 is shown in Table 21 as % of remaining vs vehicle.

TABLE 21
Relative pgRNA in the liver of PXB mice

| Group | N | % of remaining pgRNA | SEM |
|---|---|---|---|
| Vehicle | 5 | 100 | 18.07 |
| CNJ ID NO 2_1 | 3 | 79.9 | 8.19 |
| CNJ ID NO 3_1 | 5 | 75.4 | 4.91 |

Intrahepatic HBV RNA Level at Day 84

Relative pgRNA level at day 84 is shown in Table 22 as % of remaining vs vehicle.

TABLE 22
Relative HBV RNA in the liver of PXB mice

| Group | N | % of remaining intrahepatic HBV RNA | SEM |
|---|---|---|---|
| Vehicle | 5 | 100 | 13.66 |
| CNJ ID NO 2_1 | 3 | 73.7 | 7.16 |
| CNJ ID NO 3_1 | 5 | 77.0 | 5.91 |

Intrahepatic Human A1CF mRNA Level at Day 84

A1CF mRNA expression level at day 84 is shown in Table 23 as % of remaining vs vehicle.

TABLE 23
Relative human A1CF mRNA in the liver of PXB mice

| Group | N | % of remaining A1CF mRNA | SEM |
|---|---|---|---|
| Vehicle | 5 | 100 | 6.76 |
| CNJ ID NO 2_1 | 3 | 32.2 | 3.06 |
| CNJ ID NO 3_1 | 5 | 13.1 | 2.14 |

Liver Compound Exposure Level at Day 84

Absolute level of compound exposure at day 84 is shown in Table 24 as % of remaining vs vehicle.

TABLE 24
Compound exposure in the liver of PXB mice

| Group | N | Compound exposure (nmol/g) | SEM |
|---|---|---|---|
| Vehicle | 5 | 0.00 | 0.00 |
| CNJ ID NO 2_1 | 3 | 0.30 | 0.04 |
| CNJ ID NO 3_1 | 5 | 2.80 | 0.39 |

Human Albumin in Blood of PXB Mice

Human Albumin (hAlb) level at day 0 and day 84 is shown in Table 25 as % of day 0 as baseline. Although there is a slight decline of human albumin from day 0 to day 84 in all groups, the total human albumin levels that are maintained through the study indicate a liver humanization of >70%.

TABLE 25
Human Albumin in the blood of PXB mice

| | % of remaining Human Albumin | | | | | | | | |
|---|---|---|---|---|---|---|---|---|---|
| | | | | CNJ ID NO | | | CNJ ID NO | | |
| study | Saline | | | 2_1 | | | 3_1 | | |
| day | Mean | SEM | N | Mean | SEM | N | Mean | SEM | N |
| 0 | 100 | 0 | 5 | 100 | 0 | 3 | 100 | 0 | 5 |
| 84 | 64.66 | 3.03 | 5 | 70.97 | 3.37 | 3 | 66.64 | 2.12 | 5 |

Histopathological Evaluation (In Vivo Safety Data)

The livers of HBV-infected mice treated with the oligonucleotides were evaluated to assess in vivo safety of the oligonucleotides.

Half of the left kidney and a ˜5 mm thick section from the left lateral lobe of the liver was obtained from all animals at necropsy, fixed with 10% neutral-buffered formalin for at least 24 hours and up to 48 hours and processed as a paraffin-embedded kidney and liver blocks. Serial sections were prepared from both organs and the sections were stained with hematoxylin-eosin.

Histopathological evaluation was performed on all animals sacrificed on Day 84, i.e. 24 animals in total. Individual animals, including control animals showed the presence of lymphoma in liver and kidney.

There were no evident oligonucleotide related histopathological changes in the kidney or liver for any of the three oligonucleotides tested in this study (CNJ ID NO 1_1, CNJ ID NO 2_1 and CNJ ID NO 3_1).

1. An antisense oligonucleotide of 12 to 30 nucleotides in length, which comprises a contiguous nucleotide sequence of 12 to 30 nucleotides in length, wherein the contiguous nucleotide sequence is capable of binding to a target sequence in an apolipoprotein B mRNA editing enzyme catalytic subunit 1 (APOBEC1) Complementation Factor (A1CF) mRNA, wherein the target sequence is a sequence of at least 17 contiguous nucleotides within any of the following sequences:
GCCUAUCUGAGAAACUUUU (SEQ ID NO 32) (positions 2181-2199 of SEQ ID NO 45),
GAGAAAAACCUAUAAUGCCU (SEQ ID NO 42) (positions 6951-6970 of SEQ ID NO 45),
AAGUAAAAUUAACAUGUCCA (SEQ ID NO 43) (positions 16970-16989 of SEQ ID NO 45),
AAACACCACAAUCUUAAAAC (SEQ ID NO 39) (positions 26358-26377 of SEQ ID NO 45),
CAGGUAUAUAACAAGUUCA (SEQ ID NO 34) (positions 38053-38071 of SEQ ID NO 45), and AGACACACAAAACUCUAU (SEQ ID NO 44) (positions 78973-78990 of SEQ ID NO 45), and wherein the antisense oligonucleotide is capable of reducing A1CF expression.

2. The antisense oligonucleotide of claim 1, wherein the target sequence is GCCUAUCUGAGAAACUUUU (SEQ ID NO 32) (positions 2181-2199 of SEQ ID NO 45) or AAGUAAAAUUAACAUGUCC (SEQ ID NO 33) (positions 16970-16988 of SEQ ID NO 45).

3. The antisense oligonucleotide of claim 1, wherein the antisense oligonucleotide is capable of (a) reducing A1CF expression in a cell infected with hepatitis B virus (HBV), (b) reducing total intracellular HBV DNA in the cell compared to a control, (c) reducing the amount of HBV DNA secreted by the cell compared to a control, (d) reducing the amount of covalently closed circular DNA (cccDNA) in the cell compared to a control, (e) reducing the amount of pregenomic RNA (pgRNA) in the cell compared to a control, and/or (f) recruiting RNase H.

4-8. (canceled)

9. The antisense oligonucleotide of claim 1, wherein the antisense oligonucleotide is a single stranded antisense oligonucleotide.

10. The antisense oligonucleotide of claim 1, wherein the contiguous nucleotide sequence is at least 90%, at least 91%, at least 92%, at least 93%, at least 94%, at least 95%, at least 96%, at least 97%, at least 98%, at least 99% or 100% complementary to the target sequence.

11-12. (canceled)

13. The antisense oligonucleotide of claim 1, wherein the antisense oligonucleotide consists of the contiguous nucleotide sequence.

14. The antisense oligonucleotide of claim 1, wherein the sequence of the antisense oligonucleotide and/or the sequence of the contiguous nucleotide sequence comprises or consists of a sequence having at least 80%, at least 85%, at least 90%, at least 95% or 100% identity to AAAAGTTTCTCAGATAGGE (SEQ ID NO 2) or GGACATGTTAATTTTACTT (SEQ ID NO 3).

15. The antisense oligonucleotide of claim 1, wherein the antisense oligonucleotide comprises one or more sugar modified nucleosides, wherein the one or more sugar modified nucleosides are independently selected from the group consisting of 2′-O-methyl-RNA, 2′-O-methoxyethyl-RNA (MOE-RNA) and LNA nucleosides.

16. (canceled)

17. The antisense oligonucleotide of claim 15, wherein each nucleotide in the antisense oligonucleotide that does not comprise a sugar modified nucleoside comprises a DNA nucleoside.

18. The antisense oligonucleotide of claim 1, wherein the antisense oligonucleotide comprises one or more modified internucleoside linkages, wherein each modified internucleoside linkage is independently selected from the group consisting of phosphorothioate internucleoside linkages and phosphorodithioate internucleoside linkages.

19-20. (canceled)

21. An antisense oligonucleotide comprising or consisting of a compound selected from the following:
(a)
(SEQ ID NO 1, CMP ID NO 1_1)
[LR](A)[sP].[LR](G)[sP].[dR](G)[sP].[dR](C)[sP].[LR](A)[sP].[dR](T)[sP].[dR](T)[sP].
[dR](A)[sP].[dR](T)[sP].[dR](A)[sP].[dR](G)[sP].[dR](G)[sP].[dR](T)[sP].[dR](T)[sP].
[dR](T)[sP].[LR](T)[sP].[LR](T)[sP].[LR]([5meC]),
(b)
(SEQ ID NO 2, CMP ID NO 2_1)
[LR](A)[PS2].[LR](A)[sP].[dR](A)[sP].[mR](A)[sP].[LR](G)[sP].[dR](T)[sP].[dR](T)[sP].
[dR](T)[sP].[dR](C)[sP].[dR](T)[sP].[dR](C)[sP].[dR](A)[sP].[dR](G)[sP].[dR](A)[sP].
[dR](T)[sP].[dR](A)[sP].[mR](G)[sP].[LR](G)[PS2].[LR]([5meC]),
(c)
(SEQ ID NO 3, CMP ID NO 3_1)
[LR](G)[sP].[LR](G)[sP].[mR](A)[sP].[dR](C)[P].[dR](A)[sP].[dR](T)[sP].[LR](G)[sP].
[dR](T)[sP].[dR](T)[sP].[dR](A)[sP].[dR](A)[sP].[dR](T)[sP].[dR](T)[sP].[dR](T)[sP].[dR]
(T)[sP].[dR](A)[sP].[dR](C)[sP].[LR](T)[PS2].[LR](T),
(d)
(SEQ ID NO 4, CMP ID NO 4_1)
[LR](T)[PS2].[LR](G)[sP].[LR](A)[sP].[dR](A)[sP].[dR](C)[sP].[dR](T)[sP].[dR](T)[sP].
[dR](G)[sP].[dR](T)[sP].[dR](T)[sP].[dR](A)[sP].[dR](T)[sP].[dR](A)[sP].[dR](T)[sP].
[dR](A)[sP].[dR](C)[sP].[LR]([5meC])[PS2].[LR](T)[PS2].[LR](G),
(e)
(SEQ ID NO 4, CMP ID NO 4_2)
[LR](T)[PS2].[LR](G)[sP].[LR](A)[sP].[mR](A)[sP].[dR](C)[sP].[dR](T)[sP].[dR](T)[sP].
[dR](G)[sP].[dR](T)[sP].[dR](T)[sP].[dR](A)[sP].[dR](T)[sP].[dR](A)[sP].[dR](T)[sP].
[dR](A)[sP].[dR](C)[sP].[LR]([5meC])[sP].[LR](T)[PS2].[LR](G),
(f)
(SEQ ID NO 5, CMP ID NO 5_1)
[LR](T)[PS2].[LR](A)[PS2].[dR](G)[sP].[dR](A)[sP].[LR](G)[sP].[dR](T)[sP].[dR](T)[sP].
[dR](T)[sP].[dR](T)[sP].[dR](G)[sP].[dR](T)[sP].[dR](G)[sP].[dR](T)[sP].[dR](G)[sP].
[dR](T)[sP].[MOE]([5meC])[sP].[MOE](T),
(g)
(SEQ ID NO 5, CMP ID NO 5_2)
[LR](T)[PS2].[LR](A)[sP].[dR](G)[sP].[dR](A)[sP].[LR](G)[sP].[dR](T)[sP].[dR](T)[sP].
[dR](T)[sP].[dR](T)[sP].[dR](G)[sP].[dR](T)[sP].[dR](G)[sP].[dR](T)[sP].[dR](G)[sP].
[dR](T)[sP].[MOE]([5meC])[sP].[MOE](T),
(h)
(SEQ ID NO 6, CMP ID NO 6_1)
[LR](G)[sP].[LR]([5meC])[sP].[dR](A)[sP].[dR](T)[sP].[dR](T)[sP].[dR](A)[sP].[dR](T)
[sP].[dR](A)[sP].[dR](G)[sP].[dR](G)[sP].[dR](T)[sP].[dR](T)[sP].[dR](T)[sP].[LR](T)
[sP].[LR](T)[sP].[LR]([5meC])[sP].[LR](T),
(i)
(SEQ ID NO 7, CMP ID NO 7_1)
[MOE](G)[sP].[LR](G)[sP].[dR](C)[sP].[LR](A)[sP].[dR](T)[sP].[dR](T)[sP].[dR](A)[sP].
[dR](T)[sP].[dR](A)[sP].[dR](G)[sP].[dR](G)[sP].[dR](T)[sP].[dR](T)[sP].[LR](T)[sP].
[MOE](T)[sP].[MOE](T)[sP].[MOE]([5meC]),
(j)
(SEQ ID NO 7, CMP ID NO 7_2)
[LR](G)[sP].[LR](G)[sP].[dR](C)[P].[LR](A)[sP].[dR](T)[sP].[dR](T)[sP].[dR](A)[sP].
[dR](T)[sP].[mR](A)[sP].[dR](G)[sP].[dR](G)[sP].[dR](T)[sP].[dR](T)[sP].[dR](T)[sP].
[LR](T)[sP].[LR](T)[sP].[LR]([5meC]),
(k)
(SEQ ID NO 8, CMP ID NO 8_1)
[MOE](G)[sP].[MOE](G)[sP].[MOE]([5meC])[sP].[LR](A)[PS2].[dR](T)[sP].[dR](T)[sP].
[dR](A)[sP].[dR](T)[sP].[mR](A)[sP].[dR](G)[sP].[dR](G)[sP].[dR](T)[sP].[dR](T)[sP].
[LR](T)[PS2].[LR](T)[PS2].[LR](T)[PS2].[MOE]([5meC]),
(l)
(SEQ ID NO 2, CMP ID NO 2_2)
[LR](A)[PS2].[LR](A)[PS2].[dR](A)[sP].[mR](A)[sP].[LR](G)[sP].[dR](T)[sP].[dR](T)[sP]
.[dR](T)[sP].[dR](C)[sP].[dR](T)[sP].[dR](C)[sP].[dR](A)[sP].[dR](G)[sP].[dR](A)[sP].
[dR](T)[sP].[dR](A)[sP].[dR](G)[sP].[LR](G)[PS2].[LR]([5meC]),
(m)
(SEQ ID NO 9, CMP ID NO 9_1)
[LR](T)[PS2].[LR](G)[sP].[dR](G)[sP].[LR](A)[sP].[dR](C)[sP].[dR](A)[sP].[mR](U)[sP].
[dR](G)[sP].[dR](T)[sP].[LR](T)[sP].[dR](A)[sP].[dR](A)[sP].[dR](T)[sP].[dR](T)[sP].
[dR](T)[sP].[dR](T)[sP].[LR](A)[PS2].[LR]([5meC]),
(n)
(SEQ ID NO 10, CMP ID NO 10_1)
[LR](G)[PS2].[LR](G)[PS2].[dR](A)[sP].[dR](C)[sP].[dR](A)[sP].[dR](T)[sP].[LR](G)[sP]
.[mR](U)[sP].[dR](T)[sP].[mR](A)[sP].[dR](A)[sP].[dR](T)[sP].[dR](T)[sP].[dR](T)[sP].
[dR](T)[sP].[dR](A)[sP].[dR](C)[P].[LR](T)[PS2].[LR](T),
(o)
(SEQ ID NO 11, CMP ID NO 11_1)
[LR](G)[sP].[LR](T)[sP].[LR](T)[sP].[dR](T)[sP].[dR](T)[sP].[mR](A)[sP].[dR](A)[sP].
[dR](G)[sP].[dR](A)[P].[dR](T)[sP].[dR](T)[sP].[dR](G)[sP].[dR](T)[sP].[dR](G)[sP].
[dR](G)[sP].[dR](T)[sP].[dR](G)[sP].[LR](T)[sP].[LR](T)[PS2].[LR](T),
(p)
(SEQ ID NO 4, CMP ID NO 4_3)
[LR](T)[PS2].[LR](G)[sP].[LR](A)[sP].[dR](A)[sP].[dR](C)[sP].[dR](T)[sP].[dR](T)[sP].
[dR](G)[sP].[dR](T)[sP].[dR](T)[sP].[dR](A)[sP].[dR](T)[sP].[dR](A)[sP].[dR](T)[sP].
[dR](A)[sP].[dR](C)[sP].[LR]([5meC])[sP].[LR](T)[PS2].[LR](G),
(q)
(SEQ ID NO 12, CMP ID NO 12_1)
[LR](T)[PS2].[LR](A)[PS2].[dR](G)[sP].[dR](A)[sP].[LR](G)[sP].[dR](T)[sP].[dR](T)
[sP].[dR](T)[sP].[mR](U)[sP].[dR](G)[sP].[dR](T)[sP].[dR](G)[sP].[dR](T)[sP].[dR](G)
[sP].[dR](T)[sP].[MOE]([5meC])[sP].[MOE](T),
(r)
(SEQ ID NO 5, CMP ID NO 5_3)
[MOE](T)[sP].[MOE](A)[sP].[MOE](G)[sP].[MOE](A)[sP].[LR](G)[PS2].[dR](T)[sP].[dR]
(T)[sP].[dR](T)[sP].[dR](T)[sP].[dR](G)[sP].[dR](T)[sP].[dR](G)[sP].[dR](T)[sP].[dR](G)
[sP].[dR](T)[sP].[LR]([5meC])[PS2].[MOE](T),
(s)
(SEQ ID NO 13, CMP ID NO 13_1)
[MOE](T)[sP].[MOE](A)[sP].[MOE](G)[sP].[MOE](A)[sP].[LR](G)[PS2].[dR](T)[sP].[dR]
(T)[sP].[dR](T)[sP].[dR](T)[sP].[dR](G)[sP].[dR](T)[sP].[dR](G)[sP].[mR](U)[sP].[dR]
(G)[sP].[dR](T)[sP].[LR]([5meC])[PS2].[MOE](T),
(t)
(SEQ ID NO 14, CMP ID NO 14_1)
[LR](A)[PS2].[LR](T)[sP].[LR](A)[sP].[LR](G)[P].[dR](A)[sP].[dR]([PPG])[sP].[dR](T)
[sP].[dR](T)[sP].[dR](T)[sP].[dR](T)[sP].[dR](G)[sP].[dR](T)[sP].[dR](G)[sP].[dR](T)
[sP].[dR](G)[sP].[LR](T)[PS2].[LR]([5meC]),
(u)
(SEQ ID NO 15, CMP ID NO 15_1)
[LR](A)[PS2].[LR](A)[sP].[LR](A)[sP].[dR](A)[sP].[dR]([PPG])[sP].[dR](T)[sP].[dR](T)
[sP].[dR](T)[sP].[dR](C)[sP].[dR](T)[sP].[dR](C)[sP].[dR](A)[sP].[dR](G)[sP].[dR](A)
[sP].[dR](T)[sP].[dR](A)[sP].[LR](G)[sP].[LR](G)[PS2].[LR]([5meC]),
(v)
(SEQ ID NO 16, CMP ID NO 16_1)
[LR](A)[PS2].[LR](A)[sP].[LR](A)[sP].[dR](A)[sP].[dR](G)[sP].[dR](T)[sP].[dR](T)[sP].
[dR](T)[sP].[dR](C)[sP].[dR](T)[sP].[dR](C)[sP].[dR](A)[sP].[dR]([PPG])[sP].[dR](A)
[sP].[dR](T)[sP].[dR](A)[sP].[LR](G)[sP].[LR](G)[PS2].[LR]([5meC]),
(w)
(SEQ ID NO 16, CMP ID NO 16_2)
[MOE](A)[sP].[LR](A)[PS2].[LR](A)[P].[dR](A)[sP].[dR](G)[sP].[dR](T)[sP].[dR](T)[sP]
[dR](T)[sP].[dR](C)[sP].[dR](T)[sP].[dR](C)[sP].[dR](A)[sP].[dR]([PPG])[sP].[dR](A)
[sP].[dR](T)[sP].[dR](A)[sP].[LR](G)[sP].[LR](G)[PS2].[MOE]([5meC]),
(x)
(SEQ ID NO 16, CMP ID NO 16_3)
[MOE](A)[sP].[LR](A)[PS2].[LR](A)[sP].[dR](A)[sP].[dR](G)[sP].[dR](T)[sP].[dR](T)[sP]
[dR](T)[sP].[dR](C)[sP].[dR](T)[sP].[dR](C)[P].[dR](A)[sP].[dR]([PPG])[sP].[dR](A)
[sP].[dR](T)[sP].[dR](A)[sP].[LR](G)[PS2].[LR](G)[PS2].[MOE]([5meC]),
(y)
(SEQ ID NO 2, CMP ID NO 2_3)
[LR](A)[PS2].[LR](A)[sP].[dR](A)[sP].[mR](A)[sP].[LR](G)[sP].[dR](T)[sP].[dR](T)[sP].
[dR](T)[sP].[dR](C)[sP].[dR](T)[sP].[dR](C)[sP].[dR](A)[sP].[dR](G)[sP].[dR](A)[sP].
[dR](T)[sP].[dR](A)[sP].[dR](G)[P].[LR](G)[PS2].[LR]([5meC]),
(z)
(SEQ ID NO 2, CMP ID NO 2_4)
[LR](A)[PS2].[LR](A)[PS2].[dR](A)[sP].[mR](A)[sP].[LR](G)[sP].[dR](T)[sP].[dR](T)[sP]
.[dR](T)[sP].[dR](C)[sP].[dR](T)[sP].[dR](C)[sP].[dR](A)[sP].[dR](G)[sP].[dR](A)[sP].
[dR](T)[sP].[dR](A)[sP].[mR](G)[sP].[LR](G)[PS2].[LR]([5meC]),
(aa)
(SEQ ID NO 2, CMP ID NO 2_5)
[LR](A)[PS2].[LR](A)[sP].[LR](A)[sP].[dR](A)[sP].[dR](G)[sP].[dR](T)[sP].[dR](T)[sP].
[dR](T)[sP].[dR](C)[sP].[dR](T)[sP].[dR](C)[sP].[mR](A)[sP].[dR](G)[sP].[dR](A)[sP].
[dR](T)[sP].[dR](A)[sP].[LR](G)[sP].[LR](G)[PS2].[LR]([5meC]),
(ab)
(SEQ ID NO 2, CMP ID NO 2_6)
[LR](A)[PS2].[MOE](A)[sP].[MOE](A)[sP].[dR](A)[sP].[dR](G)[sP].[dR](T)[sP].[dR](T)
[sP].[dR](T)[sP].[dR](C)[sP].[dR](T)[sP].[dR](C)[sP].[mR](A)[sP].[dR](G)[sP].[dR](A)
[sP].[dR](T)[sP].[dR](A)[sP].[LR](G)[PS2].[LR](G)[PS2].[MOE]([5meC]),
(ac)
(SEQ ID NO 2, CMP ID NO 2_7)
[LR](A)[PS2].[LR](A)[sP].[LR](A)[sP].[dR](A)[sP].[dR](G)[sP].[dR](T)[sP].[dR](T)[sP].
[dR](T)[sP].[dR](C)[sP].[dR](T)[sP].[mR](C)[sP].[dR](A)[sP].[dR](G)[sP].[dR](A)[sP].
[dR](T)[sP].[dR](A)[sP].[LR](G)[sP].[LR](G)[PS2].[LR]([5meC]),
(ad)
(SEQ ID NO 2, CMP ID NO 2_8)
[MOE](A)[sP].[MOE](A)[sP].[LR](A)[PS2].[dR](A)[sP].[dR](G)[sP].[dR](T)[sP].[dR](T)
[sP].[dR](T)[sP].[dR](C)[P].[dR](T)[sP].[mR](C)[sP].[dR](A)[sP].[dR](G)[sP].[dR](A)
[sP].[dR](T)[sP].[dR](A)[sP].[LR](G)[sP].[LR](G)[PS2].[MOE]([5meC]),
(ae)
(SEQ ID NO 2, CMP ID NO 2_9)
[MOE](A)[P].[LR](A)[sP].[LR](A)[sP].[dR](A)[sP].[dR](G)[sP].[dR](T)[sP].[dR](T)[sP].
[dR](T)[sP].[dR](C)[sP].[dR](T)[sP].[dR](C)[sP].[mR](A)[sP].[dR](G)[sP].[dR](A)[sP].
[dR](T)[sP].[dR](A)[sP].[LR](G)[sP].[LR](G)[sP].[MOE]([5meC]),
(af)
(SEQ ID NO 2, CMP ID NO 2_10)
[LR](A)[PS2].[LR](A)[sP].[LR](A)[sP].[dR](A)[sP].[dR](G)[sP].[dR](T)[sP].[dR](T)[sP].
[dR](T)[sP].[mR](C)[sP].[dR](T)[sP].[dR](C)[sP].[dR](A)[sP].[dR](G)[sP].[dR](A)[sP].
[dR](T)[sP].[dR](A)[sP].[LR](G)[sP].[LR](G)[PS2].[LR]([5meC]),
(ag)
(SEQ ID NO 2, CMP ID NO 2_11)
[MOE](A)[sP].[LR](A)[sP].[LR](A)[sP].[dR](A)[P].[dR](G)[sP].[dR](T)[sP].[dR](T)[sP].
[dR](T)[sP].[mR](C)[P].[dR](T)[sP].[dR](C)[sP].[dR](A)[sP].[dR](G)[sP].[dR](A)[sP].
[dR](T)[sP].[dR](A)[sP].[LR](G)[sP].[LR](G)[PS2].[MOE]([5meC]),
(ah)
(SEQ ID NO 2, CMP ID NO 2_12)
[MOE](A)[sP].[LR](A)[PS2].[LR](A)[sP].[dR](A)[sP].[dR](G)[sP].[dR](T)[sP].[dR](T)[sP]
.[dR](T)[sP].[dR](C)[sP].[dR](T)[sP].[dR](C)[P].[dR](A)[sP].[dR](G)[sP].[dR](A)[sP].
[dR](T)[sP].[mR](A)[sP].[LR](G)[sP].[LR](G)[PS2].[MOE]([5meC]),
(ai)
(SEQ ID NO 17, CMP ID NO 17_1)
[MOE](A)[sP].[MOE](A)[sP].[LR](A)[sP].[dR](A)[sP].[dR](G)[sP].[dR](T)[sP].[dR](T)
[sP].[mR](U)[sP].[dR](C)[sP].[dR](T)[sP].[dR](C)[P].[dR](A)[sP].[dR](G)[sP].[dR](A)
[sP].[dR](T)[sP].[dR](A)[sP].[LR](G)[sP].[LR](G)[PS2].[MOE]([5meC]),
(aj)
(SEQ ID NO 18, CMP ID NO 18_1)
[LR](A)[PS2].[LR](G)[sP].[dR](G)[sP].[dR](C)[sP].[LR](A)[P].[dR](T)[sP].[dR](T)[sP].
[dR](A)[sP].[dR](T)[sP].[dR](A)[sP].[dR](G)[sP].[dR](G)[sP].[mR](U)[sP].[dR](T)[sP].
[dR](T)[sP].[LR](T)[sP].[LR](T)[PS2].[LR]([5meC]),
(ak)
(SEQ ID NO 19, CMP ID NO 19_1)
[LR](A)[PS2].[LR](G)[sP].[dR](G)[sP].[dR](C)[sP].[LR](A)[sP].[mR](U)[sP].[dR](T)[sP].
[dR](A)[sP].[dR](T)[sP].[dR](A)[sP].[dR](G)[sP].[dR](G)[sP].[dR](T)[sP].[dR](T)[sP].
[dR](T)[sP].[LR](T)[sP].[LR](T)[PS2].[LR]([5meC]),
(al)
(SEQ ID NO 20, CMP ID NO 20_1)
[LR](G)[PS2].[LR]([5meC])[P].[dR](A)[sP].[dR](T)[sP].[dR](T)[sP].[dR](A)[sP].[dR](T)
[sP].[dR](A)[sP].[dR](G)[sP].[dR](G)[sP].[dR](T)[sP].[LR](T)[sP].[dR](T)[sP].[dR](T)
[sP].[dR](T)[sP].[dR](C)[sP].[LR](T)[PS2].[LR]([5meC]),
(am)
(SEQ ID NO 20, CMP ID NO 20_2)
[LR](G)[PS2].[LR]([5meC])[sP].[mR](A)[sP].[dR](T)[sP].[dR](T)[sP].[dR](A)[sP].[dR](T)
[sP].[dR](A)[sP].[dR](G)[sP].[dR](G)[sP].[dR](T)[sP].[LR](T)[sP].[dR](T)[sP].[dR](T)
[sP].[dR](T)[sP].[dR](C)[sP].[LR](T)[PS2].[LR]([5meC]),
(an)
(SEQ ID NO 20, CMP ID NO 20_3)
[LR](G)[PS2].[LR]([5meC])[sP].[dR](A)[sP].[dR](T)[sP].[dR](T)[sP].[dR](A)[sP].[dR](T)
[sP].[mR](A)[sP].[dR](G)[sP].[dR](G)[sP].[dR](T)[sP].[LR](T)[sP].[dR](T)[sP].[dR](T)
[sP].[dR](T)[sP].[dR](C)[sP].[LR](T)[PS2].[LR]([5meC]),
(ao)
(SEQ ID NO 20, CMP ID NO 20_4)
[sP].[dR](A)[sP].[mR](G)[sP].[dR](G)[sP].[dR](T)[sP].[LR](T)[sP].[dR](T)[sP].[dR](T)
[sP].[dR](T)[sP].[dR](C)[sP].[LR](T)[PS2].[LR]([5meC]),
(ap)
(SEQ ID NO 20, CMP ID NO 20_5)
[LR](G)[PS2].[LR]([5meC])[sP].[dR](A)[sP].[dR](T)[sP].[dR](T)[sP].[dR](A)[sP].[dR](T)
[sP].[dR](A)[sP].[dR](G)[sP].[dR](G)[sP].[dR](T)[sP].[LR](T)[sP].[dR](T)[sP].[dR](T)
[sP].[dR](T)[sP].[dR](C)[sP].[LR](T)[PS2].[MOE]([5meC]),
(aq)
(SEQ ID NO 20, CMP ID NO 20_6)
[LR](G)[PS2].[LR]([5meC])[sP].[dR](A)[sP].[dR](T)[sP].[dR](T)[sP].[dR](A)[sP].[dR](T)
[sP].[dR](A)[sP].[dR](G)[sP].[mR](G)[sP].[dR](T)[sP].[LR](T)[sP].[dR](T)[sP].[dR](T)
[sP].[dR](T)[sP].[dR](C)[sP].[LR](T)[PS2].[LR]([5meC]),
(ar)
(SEQ ID NO 6, CMP ID NO 6_2)
[LR](G)[PS2].[LR]([5meC])[sP].[LR](A)[sP].[dR](T)[sP].[dR](T)[sP].[dR](A)[sP].[dR](T)
[sP].[dR](A)[sP].[dR](G)[sP].[dR](G)[sP].[dR](T)[sP].[dR](T)[sP].[dR](T)[sP].[LR](T)
[sP].[dR](T)[sP].[LR]([5meC])[PS2].[LR](T),
(as)
(SEQ ID NO 6, CMP ID NO 6_3)
[LR](G)[PS2].[LR]([5meC])[sP].[LR](A)[sP].[dR](T)[sP].[dR](T)[sP].[dR](A)[sP].[dR](T)
[sP].[mR](A)[sP].[dR](G)[sP].[dR](G)[sP].[dR](T)[sP].[dR](T)[sP].[dR](T)[sP].[LR](T)
[sP].[dR](T)[sP].[LR]([5meC])[PS2].[LR](T),
(at)
(SEQ ID NO 6, CMP ID NO 6_4)
[LR](G)[PS2].[LR]([5meC])[sP].[LR](A)[sP].[dR](T)[sP].[dR](T)[sP].[dR](A)[sP].[dR](T)
[sP].[dR](A)[sP].[mR](G)[sP].[dR](G)[sP].[dR](T)[sP].[dR](T)[sP].[dR](T)[sP].[LR](T)
[sP].[dR](T)[sP].[LR]([5meC])[PS2].[LR](T),
(au)
(SEQ ID NO 6, CMP ID NO 6_5)
[LR](G)[PS2].[LR]([5meC])[sP].[mR](A)[sP].[dR](T)[sP].[dR](T)[sP].[dR](A)[sP].[dR](T)
[sP].[dR](A)[sP].[dR](G)[sP].[dR](G)[sP].[dR](T)[sP].[dR](T)[sP].[dR](T)[sP].[LR](T)
[sP].[LR](T)[sP].[LR]([5meC])[PS2].[LR](T),
(av)
(SEQ ID NO 6, CMP ID NO 6_6)
[LR](G)[PS2].[LR]([5meC])[sP].[dR](A)[sP].[dR](T)[sP].[dR](T)[sP].[dR](A)[sP].[dR](T)
[sP].[mR](A)[sP].[dR](G)[sP].[dR](G)[sP].[dR](T)[sP].[dR](T)[sP].[dR](T)[sP].[LR](T)
[sP].[LR](T)[sP].[LR]([5meC])[PS2].[LR](T),
(aw)
(SEQ ID NO 6, CMP ID NO 6_7)
[LR](G)[PS2].[LR]([5meC])[sP].[dR](A)[sP].[dR](T)[sP].[dR](T)[sP].[dR](A)[sP].[dR](T)
[sP].[dR](A)[sP].[mR](G)[sP].[dR](G)[sP].[dR](T)[sP].[dR](T)[sP].[dR](T)[sP].[LR](T)
[sP].[LR](T)[sP].[LR]([5meC])[PS2].[LR](T),
(ax)
(SEQ ID NO 6, CMP ID NO 6_8)
[LR](G)[PS2].[LR]([5meC])[P].[dR](A)[sP].[dR](T)[sP].[dR](T)[sP].[dR](A)[sP].[dR](T)
[sP].[dR](A)[sP].[dR](G)[sP].[mR](G)[sP].[dR](T)[sP].[dR](T)[sP].[dR](T)[sP].[LR](T)
[sP].[LR](T)[sP].[LR]([5meC])[PS2].[LR](T),
(ay)
(SEQ ID NO 6, CMP ID NO 6_9)
[MOE](G)[sP].[LR]([5meC])[PS2].[dR](A)[sP].[dR](T)[sP].[dR](T)[sP].[dR](A)[sP].[dR]
(T)[sP].[dR](A)[sP].[dR](G)[sP].[dR](G)[sP].[dR](T)[sP].[dR](T)[sP].[dR](T)[sP].[LR](T)
[sP].[LR](T)[sP].[LR]([5meC])[PS2].[MOE](T),
(az)
(SEQ ID NO 6, CMP ID NO 6_10)
[MOE](G)[sP].[LR]([5meC])[PS2].[dR](A)[sP].[dR](T)[sP].[dR](T)[sP].[dR](A)[sP].[dR]
(T)[sP].[dR](A)[sP].[dR](G)[sP].[dR](G)[sP].[dR](T)[sP].[dR](T)[sP].[dR](T)[sP].[LR](T)
[sP].[LR](T)[PS2].[MOE]([5meC])[sP].[MOE](T),
(ba)
(SEQ ID NO 6, CMP ID NO 6_11)
[MOE](G)[sP].[LR]([5meC])[PS2].[dR](A)[sP].[dR](T)[sP].[dR](T)[sP].[dR](A)[sP].[dR]
(T)[sP].[dR](A)[sP].[dR](G)[sP].[dR](G)[sP].[dR](T)[sP].[dR](T)[sP].[dR](T)[sP].[LR](T)
[sP].[LR](T)[PS2].[LR]([5meC])[PS2].[MOE](T),
(bb)
(SEQ ID NO 6, CMP ID NO 6_12)
[MOE](G)[sP].[LR]([5meC])[PS2].[dR](A)[sP].[dR](T)[sP].[dR](T)[sP].[dR](A)[sP].[dR]
(T)[sP].[dR](A)[sP].[dR](G)[sP].[dR](G)[sP].[dR](T)[sP].[dR](T)[sP].[dR](T)[sP].[LR](T)
[PS2].[LR](T)[PS2].[MOE]([5meC])[sP].[MOE](T),
(bc)
(SEQ ID NO 21, CMP ID NO 21_1)
[LR](G)[PS2].[LR]([5meC])[sP].[dR](A)[sP].[dR](T)[sP].[dR](T)[sP].[dR](A)[sP].[dR](T)
[sP].[dR](A)[sP].[dR](G)[sP].[dR](G)[sP].[dR](T)[sP].[LR](T)[sP].[dR](T)[sP].[dR](T)
[sP].[mR](U)[sP].[dR](C)[sP].[LR](T)[PS2].[LR]([5meC]),
(bd)
(SEQ ID NO 22, CMP ID NO 22_1)
[LR](G)[PS2].[LR]([5meC])[sP].[dR](A)[sP].[dR](T)[sP].[dR](T)[sP].[dR](A)[sP].[dR](T)
[sP].[dR](A)[sP].[dR](G)[sP].[dR](G)[sP].[dR](T)[sP].[LR](T)[sP].[dR](T)[sP].[mR](U)
[sP].[dR](T)[sP].[dR](C)[sP].[LR](T)[PS2].[LR]([5meC]),
(be)
(SEQ ID NO 23, CMP ID NO 23_1)
[LR](G)[PS2].[LR]([5meC])[sP].[dR](A)[sP].[dR](T)[sP].[dR](T)[sP].[dR](A)[sP].[dR](T)
[sP].[dR](A)[sP].[dR](G)[sP].[dR](G)[sP].[dR](T)[sP].[LR](T)[sP].[mR](U)[sP].[dR](T)
[sP].[dR](T)[sP].[dR](C)[sP].[LR](T)[PS2].[LR]([5meC]),
(bf)
(SEQ ID NO 24, CMP ID NO 24_1)
[LR](G)[PS2].[LR]([5meC])[sP].[dR](A)[sP].[dR](T)[sP].[dR](T)[sP].[dR](A)[sP].[dR](T)
[sP].[dR](A)[sP].[dR](G)[sP].[dR](G)[sP].[dR](T)[sP].[dR](T)[sP].[mR](U)[sP].[LR](T)
[sP].[LR](T)[sP].[LR]([5meC])[PS2].[LR](T),
(bg)
(SEQ ID NO 25, CMP ID NO 25_1)
[LR](G)[PS2].[LR]([5meC])[sP].[LR](A)[sP].[dR](T)[sP].[dR](T)[sP].[dR](A)[sP].[dR](T)
[sP].[dR](A)[sP].[dR](G)[sP].[dR](G)[sP].[dR](T)[sP].[mR](U)[sP].[dR](T)[sP].[LR](T)
[sP].[dR](T)[sP].[LR]([5meC])[PS2].[LR](T),
(bh)
(SEQ ID NO 25, CMP ID NO 25_2)
[LR](G)[PS2].[LR]([5meC])[sP].[dR](A)[sP].[dR](T)[sP].[dR](T)[sP].[dR](A)[sP].[dR](T)
[sP].[dR](A)[sP].[dR](G)[sP].[dR](G)[sP].[dR](T)[sP].[mR](U)[sP].[dR](T)[sP].[LR](T)
[sP].[LR](T)[sP].[LR]([5meC])[PS2].[LR](T),
(bi)
(SEQ ID NO 26, CMP ID NO 26_1)
[LR](G)[PS2].[LR]([5meC])[sP].[dR](A)[sP].[dR](T)[sP].[dR](T)[sP].[dR](A)[sP].[dR](T)
[sP].[dR](A)[sP].[dR](G)[sP].[dR](G)[sP].[mR](U)[sP].[LR](T)[P].[dR](T)[sP].[dR](T)
[sP].[dR](T)[sP].[dR](C)[sP].[LR](T)[PS2].[LR]([5meC]),
(bj)
(SEQ ID NO 27, CMP ID NO 27_1)
[LR](G)[PS2].[LR]([5meC])[sP].[LR](A)[sP].[dR](T)[sP].[dR](T)[sP].[dR](A)[sP].[dR](T)
[sP].[dR](A)[sP].[dR](G)[sP].[dR](G)[sP].[mR](U)[sP].[dR](T)[sP].[dR](T)[sP].[LR](T)
[sP].[dR](T)[sP].[LR]([5meC])[PS2].[LR](T),
(bk)
(SEQ ID NO 27, CMP ID NO 27_2)
[LR](G)[PS2].[LR]([5meC])[sP].[dR](A)[sP].[dR](T)[sP].[dR](T)[sP].[dR](A)[sP].[dR](T)
[sP].[dR](A)[sP].[dR](G)[sP].[dR](G)[sP].[mR](U)[sP].[dR](T)[sP].[dR](T)[sP].[LR](T)
[sP].[LR](T)[sP].[LR]([5meC])[PS2].[LR](T),
(bl)
(SEQ ID NO 28, CMP ID NO 28_1)
[LR](G)[PS2].[LR]([5meC])[sP].[dR](A)[sP].[dR](T)[sP].[dR](T)[sP].[dR](A)[sP].[mR]
(U)[sP].[dR](A)[sP].[dR](G)[sP].[dR](G)[sP].[dR](T)[sP].[dR](T)[sP].[dR](T)[sP].[LR](T)
[sP].[LR](T)[sP].[LR]([5meC])[PS2].[LR](T),
(bm)
(SEQ ID NO 29, CMP ID NO 29_1)
[LR](G)[PS2].[LR]([5meC])[sP].[dR](A)[sP].[dR](T)[sP].[mR](U)[sP].[dR](A)[sP].[dR]
(T)[P].[dR](A)[sP].[dR](G)[sP].[dR](G)[sP].[dR](T)[sP].[dR](T)[sP].[dR](T)[sP].[LR](T)
[sP].[LR](T)[sP].[LR]([5meC])[PS2].[LR](T),
(bn)
(SEQ ID NO 30, CMP ID NO 30_1)
[LR](G)[PS2].[LR]([5meC])[sP].[dR](A)[sP].[mR](U)[sP].[dR](T)[sP].[dR](A)[sP].[dR]
(T)[sP].[dR](A)[sP].[dR](G)[sP].[dR](G)[sP].[dR](T)[sP].[dR](T)[sP].[dR](T)[sP].[LR](T)
[sP].[LR](T)[sP].[LR]([5meC])[PS2].[LR](T),
(bo)
(SEQ ID NO 11, CMP ID NO 11_2)
[LR](G)[sP].[LR](T)[sP].[LR](T)[sP].[dR](T)[sP].[dR](T)[sP].[dR](A)[sP].[dR](A)[sP].
[mR](G)[sP].[dR](A)[sP].[dR](T)[sP].[dR](T)[sP].[dR](G)[sP].[dR](T)[sP].[dR](G)[sP].
[dR](G)[sP].[dR](T)[sP].[dR](G)[sP].[LR](T)[sP].[LR](T)[sP].[LR](T),
and
(bp)
(SEQ ID NO 11, CMP ID NO 11_3)
[LR](G)[sP].[LR](T)[sP].[LR](T)[sP].[dR](T)[sP].[dR](T)[sP].[dR](A)[sP].[dR](A)[sP].
[mR](G)[sP].[dR](A)[sP].[dR](T)[sP].[dR](T)[sP].[dR](G)[sP].[dR](T)[sP].[dR](G)[sP].
[dR](G)[sP].[dR](T)[sP].[dR](G)[P].[LR](T)[sP].[LR](T)[PS2].[LR](T),
wherein
(A) is a nucleoside comprising an adenine nucleobase,
(C) is a nucleoside comprising a cytosine nucleobase,
([5meC]) is a nucleoside comprising a 5-methyl cytosine nucleobase,
([PPG]) is a nucleoside comprising a 7-deaza-8-aza guanine nucleobase,
(G) is a nucleoside comprising a guanine nucleobase,
(U) is a nucleoside comprising a uracil nucleobase,
(T) is a nucleoside comprising a thymine nucleobase,
[LR] indicates that the following nucleoside is a beta-D-oxy-LNA nucleoside,
[dR] indicates that the following nucleoside is a DNA nucleoside,
[mR] indicates that the following nucleoside is a 2′-O-methyl RNA nucleoside,
[MOE] indicates that the following nucleoside is a 2′-O-methoxyethyl RNA (MOE RNA) nucleoside,
[sP] indicates a phosphorothioate internucleoside linkage between the preceding and following nucleoside, and
[PS2] indicates a phosphorodithioate internucleoside linkage between the preceding and following nucleoside.

22. An antisense oligonucleotide conjugate comprising the antisense oligonucleotide of claim 21 covalently attached to at least one conjugate moiety, wherein the conjugate moiety is an N-acetylgalactosamine (GalNAc) conjugate moiety, preferably wherein the GalNAc conjugate moiety is aminohexyl conjugated tri(N-acetyl-galactosamine) as depicted in FIG. 8.

23-24. (canceled)

25. An antisense oligonucleotide conjugate selected from the following:
(a)
(SEQ ID NO 1, CMP ID NO 1_1, CNJ ID NO 1_1)
[5gn2c6]P.[dR](C)P.[dR](A)P.[LR](A)[sP].[LR](G)[sP].[dR](G)[sP].[dR](C)[sP].[LR]
(A)[sP].[dR](T)[sP].[dR](T)[sP].[dR](A)[sP].[dR](T)[sP].[dR](A)[sP].[dR](G)[sP].[dR](G)
[sP].[dR](T)[sP].[dR](T)[sP].[dR](T)[sP].[LR](T)[sP].[LR](T)[sP].[LR]([5meC]),
(b)
(SEQ ID NO 2, CMP ID NO 2_1, CNJ ID NO 2_1)
[5gn2c6]P.[dR](C)P.[dR](A)P.[LR](A)[PS2].[LR](A)[sP].[dR](A)[sP].[mR](A)[sP].[LR]
(G)[sP].[dR](T)[sP].[dR](T)[sP].[dR](T)[sP].[dR](C)[sP].[dR](T)[sP].[dR](C)[sP].[dR](A)
[sP].[dR](G)[sP].[dR](A)[sP].[dR](T)[sP].[dR](A)[P].[mR](G)[sP].[LR](G)[PS2].[LR]([5meC]),
(c)
(SEQ ID NO 3, CMP ID NO 3_1, CNJ ID NO 3_1)
[5gn2c6]P.[dR](C)P.[dR](A)P.[LR](G)[sP].[LR](G)[sP].[mR](A)[sP].[dR](C)[sP].[dR]
(A)[sP].[dR](T)[sP].[LR](G)[sP].[dR](T)[sP].[dR](T)[sP].[dR](A)[sP].[dR](A)[sP].[dR](T)
[sP].[dR](T)[sP].[dR](T)[sP].[dR](T)[sP].[dR](A)[sP].[dR](C)[sP].[LR](T)[PS2].[LR](T),
(d)
(SEQ ID NO 4, CMP ID NO 4_1, CNJ ID NO 4_1)
[5gn2c6]P.[dR](C)P.[dR](A)P.[LR](T)[PS2].[LR](G)[sP].[LR](A)[sP].[dR](A)[sP].[dR]
(C)[sP].[dR](T)[sP].[dR](T)[sP].[dR](G)[sP].[dR](T)[sP].[dR](T)[sP].[dR](A)[sP].[dR](T)
[sP].[dR](A)[sP].[dR](T)[sP].[dR](A)[sP].[dR](C)[sP].[LR]([5meC])[PS2].[LR](T)[PS2].[LR](G),
(e)
(SEQ ID NO 4, CMP ID NO 4_2, CNJ ID NO 4_2)
[5gn2c6]P.[dR](C)P.[dR](A)P.[LR](T)[PS2].[LR](G)[sP].[LR](A)[sP].[mR](A)[sP].[dR]
(C)[sP].[dR](T)[sP].[dR](T)[sP].[dR](G)[sP].[dR](T)[sP].[dR](T)[sP].[dR](A)[sP].[dR](T)
[sP].[dR](A)[sP].[dR](T)[sP].[dR](A)[sP].[dR](C)[sP].[LR]([5meC])[sP].[LR](T)[PS2].[LR](G),
(f)
(SEQ ID NO 5, CMP ID NO 5_1, CNJ ID NO 5_1)
[5gn2c6]P.[dR](C)P.[dR](A)P.[LR](T)[PS2].[LR](A)[PS2].[dR](G)[sP].[dR](A)[sP].
[LR](G)[sP].[dR](T)[sP].[dR](T)[sP].[dR](T)[sP].[dR](T)[sP].[dR](G)[sP].[dR](T)[sP].
[dR](G)[sP].[dR](T)[sP].[dR](G)[sP].[dR](T)[sP].[MOE]([5meC])[sP].[MOE](T),
(g)
(SEQ ID NO 5, CMP ID NO 5_2, CNJ ID NO 5_2)
[5gn2c6]P.[dR](C)P.[dR](A)P.[LR](T)[PS2].[LR](A)[sP].[dR](G)[sP].[dR](A)[sP].[LR]
(G)[sP].[dR](T)[sP].[dR](T)[sP].[dR](T)[sP].[dR](T)[sP].[dR](G)[sP].[dR](T)[sP].[dR](G)
[sP].[dR](T)[sP].[dR](G)[sP].[dR](T)[sP].[MOE]([5meC])[sP].[MOE](T),
(h)
(SEQ ID NO 6, CMP ID NO 6_1, CNJ ID NO 6_1)
[5gn2c6]P.[dR](C)P.[dR](A)P.[LR](G)[sP].[LR]([5meC])[sP].[dR](A)[sP].[dR](T)[sP].
[dR](T)[sP].[dR](A)[sP].[dR](T)[sP].[dR](A)[sP].[dR](G)[sP].[dR](G)[sP].[dR](T)[sP].
[dR](T)[sP].[dR](T)[sP].[LR](T)[P].[LR](T)[sP].[LR]([5meC])[sP].[LR](T),
(i)
(SEQ ID NO 7, CMP ID NO 7_1, CNJ ID NO 7_1)
[5gn2c6]P.[dR](C)P.[dR](A)P.[MOE](G)[P].[LR](G)[sP].[dR](C)[sP].[LR](A)[sP].[dR]
(T)[sP].[dR](T)[sP].[dR](A)[P].[dR](T)[sP].[dR](A)[sP].[dR](G)[sP].[dR](G)[sP].[dR](T)
[sP].[dR](T)[sP].[LR](T)[sP].[MOE](T)[sP].[MOE](T)[sP].[MOE]([5meC]),
(j)
(SEQ ID NO 7, CMP ID NO 7_2, CNJ ID NO 7_2)
[5gn2c6]P.[dR](C)P.[dR](A)P.[LR](G)[sP].[LR](G)[sP].[dR](C)[sP].[LR](A)[sP].[dR](T)
[sP].[dR](T)[sP].[dR](A)[sP].[dR](T)[sP].[mR](A)[sP].[dR](G)[sP].[dR](G)[sP].[dR](T)
[sP].[dR](T)[sP].[dR](T)[sP].[LR](T)[sP].[LR](T)[sP].[LR]([5meC]),
(k)
(SEQ ID NO 8, CMP ID NO 8_1, CNJ ID NO 8_1)
[5gn2c6]P.[dR](C)P.[dR](A)P.[MOE](G)[sP].[MOE](G)[sP].[MOE]([5meC])[sP].[LR](A)
[PS2].[dR](T)[sP].[dR](T)[sP].[dR](A)[sP].[dR](T)[sP].[mR](A)[sP].[dR](G)[sP].[dR](G)
[sP].[dR](T)[sP].[dR](T)[sP].[LR](T)[PS2].[LR](T)[PS2].[LR](T)[PS2].[MOE]([5meC]),
(l)
(SEQ ID NO 2, CMP ID NO 2_2, CNJ ID NO 2_2)
[5gn2c6]P.[dR](C)P.[dR](A)P.[LR](A)[PS2].[LR](A)[PS2].[dR](A)[sP].[mR](A)[sP].[LR]
(G)[sP].[dR](T)[sP].[dR](T)[sP].[dR](T)[sP].[dR](C)[sP].[dR](T)[sP].[dR](C)[sP].[dR](A)
[sP].[dR](G)[sP].[dR](A)[sP].[dR](T)[sP].[dR](A)[sP].[dR](G)[sP].[LR](G)[PS2].[LR]([5meC]),
(m)
(SEQ ID NO 9, CMP ID NO 9_1, CNJ ID NO 9_1)
[5gn2c6]P.[dR](C)P.[dR](A)P.[LR](T)[PS2].[LR](G)[sP].[dR](G)[sP].[LR](A)[sP].[dR]
(C)[sP].[dR](A)[sP].[mR](U)[sP].[dR](G)[sP].[dR](T)[sP].[LR](T)[sP].[dR](A)[sP].[dR]
(A)[sP].[dR](T)[sP].[dR](T)[sP].[dR](T)[sP].[dR](T)[sP].[LR](A)[PS2].[LR]([5meC]),
(n)
(SEQ ID NO 10, CMP ID NO 10_1, CNJ ID NO 10_1)
[5gn2c6]P.[dR](C)P.[dR](A)P.[LR](G)[PS2].[LR](G)[PS2].[dR](A)[sP].[dR](C)[sP].[dR]
(A)[sP].[dR](T)[sP].[LR](G)[sP].[mR](U)[sP].[dR](T)[sP].[mR](A)[sP].[dR](A)[sP].[dR]
(T)[sP].[dR](T)[sP].[dR](T)[sP].[dR](T)[sP].[dR](A)[sP].[dR](C)[sP].[LR](T)[PS2].[LR](T),
(o)
(SEQ ID NO 11, CMP ID NO 11_1, CNJ ID NO 11_1)
[5gn2c6]P.[dR](C)P.[dR](A)P.[LR](G)[sP].[LR](T)[P].[LR](T)[sP].[dR](T)[sP].[dR](T)[sP].
[mR](A)[sP].[dR](A)[sP].[dR](G)[sP].[dR](A)[sP].[dR](T)[sP].[dR](T)[sP].[dR](G)[sP].[dR](T)
[sP].[dR](G)[sP].[dR](G)[sP].[dR](T)[sP].[dR](G)[sP].[LR](T)[sP].[LR](T)[PS2].[LR](T),
(p)
(SEQ ID NO 4, CMP ID NO 4_3, CNJ ID NO 4_3)
[5gn2c6]P.[dR](C)P.[dR](A)P.[LR](T)[PS2].[LR](G)[sP].[LR](A)[sP].[dR](A)[sP].[dR](C)
[sP].[dR](T)[sP].[dR](T)[sP].[dR](G)[sP].[dR](T)[sP].[dR](T)[sP].[dR](A)[sP].[dR](T)[sP]
.[dR](A)[sP].[dR](T)[sP].[dR](A)[sP].[dR](C)[sP].[LR]([5meC])[sP].[LR](T)[PS2].[LR](G),
(q)
(SEQ ID NO 12, CMP ID NO 12_1, CNJ ID NO 12_1)
[5gn2c6]P.[dR](C)P.[dR](A)P.[LR](T)[PS2].[LR](A)[PS2].[dR](G)[sP].[dR](A)[sP].[LR]
(G)[sP].[dR](T)[sP].[dR](T)[sP].[dR](T)[sP].[mR](U)[sP].[dR](G)[sP].[dR](T)[sP].[dR]
(G)[sP].[dR](T)[sP].[dR](G)[sP].[dR](T)[sP].[MOE]([5meC])[sP].[MOE](T),
(r)
(SEQ ID NO 5, CMP ID NO 5_3, CNJ ID NO 5_3)
[5gn2c6]P.[dR](C)P.[dR](A)P.[MOE](T)[sP].[MOE](A)[sP].[MOE](G)[sP].[MOE](A)[sP]
.[LR](G)[PS2].[dR](T)[sP].[dR](T)[sP].[dR](T)[sP].[dR](T)[sP].[dR](G)[sP].[dR](T)[sP].
[dR](G)[sP].[dR](T)[sP].[dR](G)[sP].[dR](T)[sP].[LR]([5meC])[PS2].[MOE](T),
(s)
(SEQ ID NO 13, CMP ID NO 13_1, CNJ ID NO 13_1)
[5gn2c6]P.[dR](C)P.[dR](A)P.[MOE](T)[sP].[MOE](A)[sP].[MOE](G)[sP].[MOE](A)[sP]
.[LR](G)[PS2].[dR](T)[sP].[dR](T)[sP].[dR](T)[sP].[dR](T)[sP].[dR](G)[sP].[dR](T)[sP].
[dR](G)[sP].[mR](U)[sP].[dR](G)[sP].[dR](T)[sP].[LR]([5meC])[PS2].[MOE](T),
(t)
(SEQ ID NO 14, CMP ID NO 14_1, CNJ ID NO 14_1)
[5gn2c6]P.[dR](C)P.[dR](A)P.[LR](A)[PS2].[LR](T)[sP].[LR](A)[sP].[LR](G)[sP].[dR](A)
[sP].[dR]([PPG])[sP].[dR](T)[sP].[dR](T)[sP].[dR](T)[sP].[dR](T)[sP].[dR](G)[sP].[dR]
(T)[sP].[dR](G)[sP].[dR](T)[sP].[dR](G)[sP].[LR](T)[PS2].[LR]([5meC]),
(u)
(SEQ ID NO 15, CMP ID NO 15_1, CNJ ID NO 15_1)
[5gn2c6]P.[dR](C)P.[dR](A)P.[LR](A)[PS2].[LR](A)[sP].[LR](A)[P].[dR](A)[sP].[dR]
([PPG])[sP].[dR](T)[sP].[dR](T)[sP].[dR](T)[sP].[dR](C)[sP].[dR](T)[sP].[dR](C)[sP].[dR]
(A)[sP].[dR](G)[sP].[dR](A)[sP].[dR](T)[P].[dR](A)[sP].[LR](G)[sP].[LR](G)[PS2].[LR]([5meC]),
(v)
(SEQ ID NO 16, CMP ID NO 16_1, CNJ ID NO 16_1)
[5gn2c6]P.[dR](C)P.[dR](A)P.[LR](A)[PS2].[LR](A)[sP].[LR](A)[sP].[dR](A)[sP].[dR](G)
[sP].[dR](T)[sP].[dR](T)[sP].[dR](T)[sP].[dR](C)[sP].[dR](T)[sP].[dR](C)[sP].[dR](A)[sP].
[dR]([PPG])[sP].[dR](A)[sP].[dR](T)[sP].[dR](A)[sP].[LR](G)[sP].[LR](G)[PS2].[LR]([5meC]),
(w)
(SEQ ID NO 16, CMP ID NO 16_2, CNJ ID NO 16_2)
[5gn2c6]P.[dR](C)P.[dR](A)P.[MOE](A)[sP].[LR](A)[PS2].[LR](A)[sP].[dR](A)[sP].[dR](G)
[sP].[dR](T)[sP].[dR](T)[sP].[dR](T)[sP].[dR](C)[sP].[dR](T)[sP].[dR](C)[sP].[dR](A)[sP].
[dR]([PPG])[sP].[dR](A)[sP].[dR](T)[sP].[dR](A)[sP].[LR](G)[sP].[LR](G)[PS2].[MOE]([5meC]),
(x)
(SEQ ID NO 16, CMP ID NO 16_3, CNJ ID NO 16_3)
[5gn2c6]P.[dR](C)P.[dR](A)P.[MOE](A)[sP].[LR](A)[PS2].[LR](A)[sP].[dR](A)[sP].[dR](G)
[sP].[dR](T)[sP].[dR](T)[sP].[dR](T)[sP].[dR](C)[sP].[dR](T)[sP].[dR](C)[sP].[dR](A)[sP].
[dR]([PPG])[sP].[dR](A)[sP].[dR](T)[sP].[dR](A)[sP].[LR](G)[PS2].[LR](G)[PS2].[MOE]([5meC]),
(y)
(SEQ ID NO 2, CMP ID NO 2_3, CNJ ID NO 2_3)
[5gn2c6]P.[dR](C)P.[dR](A)P.[LR](A)[PS2].[LR](A)[sP].[dR](A)[sP].[mR](A)[sP].[LR]
(G)[sP].[dR](T)[sP].[dR](T)[sP].[dR](T)[sP].[dR](C)[sP].[dR](T)[sP].[dR](C)[sP].[dR](A)[s
P].[dR](G)[sP].[dR](A)[sP].[dR](T)[sP].[dR](A)[sP].[dR](G)[sP].[LR](G)[PS2].[LR]([5meC]),
(z)
(SEQ ID NO 2, CMP ID NO 2_4, CNJ ID NO 2_4)
[5gn2c6]P.[dR](C)P.[dR](A)P.[LR](A)[PS2].[LR](A)[PS2].[dR](A)[sP].[mR](A)[sP].[LR]
(G)[sP].[dR](T)[sP].[dR](T)[sP].[dR](T)[sP].[dR](C)[sP].[dR](T)[sP].[dR](C)[sP].[dR](A)
[sP].[dR](G)[sP].[dR](A)[sP].[dR](T)[sP].[dR](A)[sP].[mR](G)[sP].[LR](G)[PS2].[LR]([5meC]),
(aa)
(SEQ ID NO 2, CMP ID NO 2_5, CNJ ID NO 2_5)
[5gn2c6]P.[dR](C)P.[dR](A)P.[LR](A)[PS2].[LR](A)[sP].[LR](A)[sP].[dR](A)[sP].[dR](G)
[sP].[dR](T)[sP].[dR](T)[sP].[dR](T)[sP].[dR](C)[sP].[dR](T)[sP].[dR](C)[sP].[mR](A)
[sP].[dR](G)[sP].[dR](A)[sP].[dR](T)[sP].[dR](A)[sP].[LR](G)[sP].[LR](G)[PS2].[LR]([5meC]),
(ab)
(SEQ ID NO 2, CMP ID NO 2_6, CNJ ID NO 2_6)
[5gn2c6]P.[dR](C)P.[dR](A)P.[LR](A)[PS2].[MOE](A)[sP].[MOE](A)[sP].[dR](A)[sP].
[dR](G)[sP].[dR](T)[sP].[dR](T)[sP].[dR](T)[sP].[dR](C)[sP].[dR](T)[sP].[dR](C)[sP].
[mR](A)[sP].[dR](G)[sP].[dR](A)[sP].[dR](T)[sP].[dR](A)[sP].[LR](G)[PS2].[LR](G)
[PS2].[MOE]([5meC]),
(ac)
(SEQ ID NO 2, CMP ID NO 2_7, CNJ ID NO 2_7)
[5gn2c6]P.[dR](C)P.[dR](A)P.[LR](A)[PS2].[LR](A)[sP].[LR](A)[sP].[dR](A)[sP].[dR](G)
[sP].[dR](T)[sP].[dR](T)[sP].[dR](T)[sP].[dR](C)[sP].[dR](T)[sP].[mR](C)[sP].[dR](A)
[sP].[dR](G)[sP].[dR](A)[sP].[dR](T)[sP].[dR](A)[sP].[LR](G)[sP].[LR](G)[PS2].[LR]([5meC]),
(ad)
(SEQ ID NO 2, CMP ID NO 2_8, CNJ ID NO 2_8)
[5gn2c6]P.[dR](C)P.[dR](A)P.[MOE](A)[sP].[MOE](A)[P].[LR](A)[PS2].[dR](A)[sP].
[dR](G)[sP].[dR](T)[sP].[dR](T)[sP].[dR](T)[sP].[dR](C)[sP].[dR](T)[sP].[mR](C)[sP].
[dR](A)[sP].[dR](G)[sP].[dR](A)[sP].[dR](T)[sP].[dR](A)[sP].[LR](G)[sP].[LR](G)[PS2].
[MOE]([5meC]),
(ae)
(SEQ ID NO 2, CMP ID NO 2_9, CNJ ID NO 2_9)
[5gn2c6]P.[dR](C)P.[dR](A)P.[MOE](A)[sP].[LR](A)[P].[LR](A)[sP].[dR](A)[sP].[dR]
(G)[sP].[dR](T)[sP].[dR](T)[sP].[dR](T)[sP].[dR](C)[sP].[dR](T)[sP].[dR](C)[sP].[mR]
(A)[sP].[dR](G)[sP].[dR](A)[sP].[dR](T)[sP].[dR](A)[sP].[LR](G)[sP].[LR](G)[sP].[MOE]
([5meC]),
(af)
(SEQ ID NO 2, CMP ID NO 2_10, CNJ ID NO 2_10)
[5gn2c6]P.[dR](C)P.[dR](A)P.[LR](A)[PS2].[LR](A)[sP].[LR](A)[sP].[dR](A)[sP].[dR](G)
[sP].[dR](T)[sP].[dR](T)[sP].[dR](T)[sP].[mR](C)[sP].[dR](T)[sP].[dR](C)[sP].[dR](A)
[sP].[dR](G)[sP].[dR](A)[sP].[dR](T)[sP].[dR](A)[sP].[LR](G)[sP].[LR](G)[PS2].[LR]
([5meC]),
(ag)
(SEQ ID NO 2, CMP ID NO 2_11, CNJ ID NO 2_11)
[5gn2c6]P.[dR](C)P.[dR](A)P.[MOE](A)[sP].[LR](A)[sP].[LR](A)[sP].[dR](A)[sP].[dR]
(G)[sP].[dR](T)[sP].[dR](T)[sP].[dR](T)[sP].[mR](C)[sP].[dR](T)[sP].[dR](C)[sP].[dR]
(A)[sP].[dR](G)[sP].[dR](A)[sP].[dR](T)[sP].[dR](A)[sP].[LR](G)[sP].[LR](G)[PS2].
[MOE]([5meC]),
(ah)
(SEQ ID NO 2, CMP ID NO 2_12, CNJ ID NO 2_12)
[5gn2c6]P.[dR](C)P.[dR](A)P.[MOE](A)[sP].[LR](A)[PS2].[LR](A)[sP].[dR](A)[sP].[dR]
(G)[sP].[dR](T)[sP].[dR](T)[sP].[dR](T)[sP].[dR](C)[sP].[dR](T)[sP].[dR](C)[sP].[dR](A)
[sP].[dR](G)[sP].[dR](A)[sP].[dR](T)[sP].[mR](A)[sP].[LR](G)[sP].[LR](G)[PS2].[MOE]
([5meC]),
(ai)
(SEQ ID NO 17, CMP ID NO 17_1, CNJ ID NO 17_1)
[5gn2c6]P.[dR](C)P.[dR](A)P.[MOE](A)[sP].[MOE](A)[sP].[LR](A)[sP].[dR](A)[sP].[dR]
(G)[sP].[dR](T)[sP].[dR](T)[sP].[mR](U)[sP].[dR](C)[sP].[dR](T)[sP].[dR](C)[sP].[dR]
(A)[sP].[dR](G)[sP].[dR](A)[sP].[dR](T)[sP].[dR](A)[sP].[LR](G)[sP].[LR](G)[PS2].
[MOE]([5meC]),
(aj)
(SEQ ID NO 18, CMP ID NO 18_1, CNJ ID NO 18_1)
[5gn2c6]P.[dR](C)P.[dR](A)P.[LR](A)[PS2].[LR](G)[sP].[dR](G)[sP].[dR](C)[sP].[LR]
(A)[sP].[dR](T)[sP].[dR](T)[sP].[dR](A)[sP].[dR](T)[sP].[dR](A)[sP].[dR](G)[sP].[dR](G)
[sP].[mR](U)[sP].[dR](T)[sP].[dR](T)[sP].[LR](T)[sP].[LR](T)[PS2].[LR]([5meC]),
(ak)
(SEQ ID NO 19, CMP ID NO 19_1, CNJ ID NO 19_1)
[5gn2c6]P.[dR](C)P.[dR](A)P.[LR](A)[PS2].[LR](G)[sP].[dR](G)[sP].[dR](C)[sP].[LR]
(A)[sP].[mR](U)[sP].[dR](T)[sP].[dR](A)[sP].[dR](T)[sP].[dR](A)[sP].[dR](G)[sP].[dR]
(G)[sP].[dR](T)[sP].[dR](T)[sP].[dR](T)[sP].[LR](T)[sP].[LR](T)[PS2].[LR]([5meC]),
(al)
(SEQ ID NO 20, CMP ID NO 20_1, CNJ ID NO 20_1)
[5gn2c6]P.[dR](C)P.[dR](A)P.[LR](G)[PS2].[LR]([5meC])[sP].[dR](A)[sP].[dR](T)[sP].
[dR](T)[sP].[dR](A)[sP].[dR](T)[sP].[dR](A)[sP].[dR](G)[sP].[dR](G)[sP].[dR](T)[sP].
[LR](T)[sP].[dR](T)[sP].[dR](T)[sP].[dR](T)[sP].[dR](C)[sP].[LR](T)[PS2].[LR]([5meC]),
(am)
(SEQ ID NO 20, CMP ID NO 20_2, CNJ ID NO 20_2)
[5gn2c6]P.[dR](C)P.[dR](A)P.[LR](G)[PS2].[LR]([5meC])[sP].[mR](A)[sP].[dR](T)[sP].
[dR](T)[sP].[dR](A)[sP].[dR](T)[sP].[dR](A)[sP].[dR](G)[sP].[dR](G)[sP].[dR](T)[sP].
[LR](T)[sP].[dR](T)[sP].[dR](T)[sP].[dR](T)[sP].[dR](C)[sP].[LR](T)[PS2].[LR]([5meC]),
(an)
(SEQ ID NO 20, CMP ID NO 20_3, CNJ ID NO 20_3)
[5gn2c6]P.[dR](C)P.[dR](A)P.[LR](G)[PS2].[LR]([5meC])[sP].[dR](A)[sP].[dR](T)[sP].
[dR](T)[sP].[dR](A)[sP].[dR](T)[sP].[mR](A)[sP].[dR](G)[sP].[dR](G)[sP].[dR](T)[sP].
[LR](T)[sP].[dR](T)[sP].[dR](T)[sP].[dR](T)[sP].[dR](C)[sP].[LR](T)[PS2].[LR]([5meC]),
(ao)
(SEQ ID NO 20, CMP ID NO 20_4, CNJ ID NO 20_4)
[5gn2c6]P.[dR](C)P.[dR](A)P.[LR](G)[PS2].[LR]([5meC])[sP].[dR](A)[sP].[dR](T)[sP].
[dR](T)[sP].[dR](A)[sP].[dR](T)[sP].[dR](A)[sP].[mR](G)[sP].[dR](G)[sP].[dR](T)[sP].
[LR](T)[sP].[dR](T)[sP].[dR](T)[sP].[dR](T)[sP].[dR](C)[sP].[LR](T)[PS2].[LR]([5meC]),
(ap)
(SEQ ID NO 20, CMP ID NO 20_5, CNJ ID NO 20_5)
[5gn2c6]P.[dR](C)P.[dR](A)P.[LR](G)[PS2].[LR]([5meC])[sP].[dR](A)[sP].[dR](T)[sP].
[dR](T)[sP].[dR](A)[sP].[dR](T)[sP].[dR](A)[sP].[dR](G)[sP].[dR](G)[sP].[dR](T)[sP].
[LR](T)[sP].[dR](T)[sP].[dR](T)[sP].[dR](T)[sP].[dR](C)[sP].[LR](T)[PS2].[MOE]
([5meC]),
(aq)
(SEQ ID NO 20, CMP ID NO 20_6, CNJ ID NO 20_6)
[5gn2c6]P.[dR](C)P.[dR](A)P.[LR](G)[PS2].[LR]([5meC])[sP].[dR](A)[sP].[dR](T)[sP].
[dR](T)[sP].[dR](A)[sP].[dR](T)[sP].[dR](A)[sP].[dR](G)[sP].[mR](G)[sP].[dR](T)[sP].
[LR](T)[P].[dR](T)[sP].[dR](T)[sP].[dR](T)[sP].[dR](C)[sP].[LR](T)[PS2].[LR]([5meC]),
(ar)
(SEQ ID NO 6, CMP ID NO 6_2, CNJ ID NO 6_2)
[5gn2c6]P.[dR](C)P.[dR](A)P.[LR](G)[PS2].[LR]([5meC])[P].[LR](A)[sP].[dR](T)[sP].
[dR](T)[sP].[dR](A)[sP].[dR](T)[sP].[dR](A)[sP].[dR](G)[sP].[dR](G)[sP].[dR](T)[sP].
[dR](T)[sP].[dR](T)[sP].[LR](T)[sP].[dR](T)[sP].[LR]([5meC])[PS2].[LR](T),
(as)
(SEQ ID NO 6, CMP ID NO 6_3, CNJ ID NO 6_3)
[5gn2c6]P.[dR](C)P.[dR](A)P.[LR](G)[PS2].[LR]([5meC])[sP].[LR](A)[sP].[dR](T)[sP].
[dR](T)[sP].[dR](A)[sP].[dR](T)[sP].[mR](A)[sP].[dR](G)[sP].[dR](G)[sP].[dR](T)[sP].
[dR](T)[sP].[dR](T)[sP].[LR](T)[sP].[dR](T)[sP].[LR]([5meC])[PS2].[LR](T),
(at)
(SEQ ID NO 6, CMP ID NO 6_4, CNJ ID NO 6_4)
[5gn2c6]P.[dR](C)P.[dR](A)P.[LR](G)[PS2].[LR]([5meC])[sP].[LR](A)[sP].[dR](T)[sP].
[dR](T)[sP].[dR](A)[sP].[dR](T)[sP].[dR](A)[sP].[mR](G)[sP].[dR](G)[sP].[dR](T)[sP].
[dR](T)[sP].[dR](T)[sP].[LR](T)[sP].[dR](T)[sP].[LR]([5meC])[PS2].[LR](T),
(au)
(SEQ ID NO 6, CMP ID NO 6_5, CNJ ID NO 6_5)
[5gn2c6]P.[dR](C)P.[dR](A)P.[LR](G)[PS2].[LR]([5meC])[sP].[mR](A)[sP].[dR](T)[sP].
[dR](T)[sP].[dR](A)[sP].[dR](T)[sP].[dR](A)[sP].[dR](G)[sP].[dR](G)[sP].[dR](T)[sP].
[dR](T)[sP].[dR](T)[sP].[LR](T)[sP].[LR](T)[sP].[LR]([5meC])[PS2].[LR](T),
(av)
(SEQ ID NO 6, CMP ID NO 6_6, CNJ ID NO 6_6)
[5gn2c6]P.[dR](C)P.[dR](A)P.[LR](G)[PS2].[LR]([5meC])[sP].[dR](A)[sP].[dR](T)[sP].
[dR](T)[sP].[dR](A)[sP].[dR](T)[sP].[mR](A)[sP].[dR](G)[sP].[dR](G)[sP].[dR](T)[sP].
[dR](T)[sP].[dR](T)[sP].[LR](T)[sP].[LR](T)[sP].[LR]([5meC])[PS2].[LR](T),
(aw)
(SEQ ID NO 6, CMP ID NO 6_7, CNJ ID NO 6_7)
[5gn2c6]P.[dR](C)P.[dR](A)P.[LR](G)[PS2].[LR]([5meC])[sP].[dR](A)[sP].[dR](T)[sP].
[dR](T)[sP].[dR](A)[sP].[dR](T)[sP].[dR](A)[sP].[mR](G)[sP].[dR](G)[sP].[dR](T)[sP].
[dR](T)[sP].[dR](T)[sP].[LR](T)[sP].[LR](T)[sP].[LR]([5meC])[PS2].[LR](T),
(ax)
(SEQ ID NO 6, CMP ID NO 6_8, CNJ ID NO 6_8)
[5gn2c6]P.[dR](C)P.[dR](A)P.[LR](G)[PS2].[LR]([5meC])[sP].[dR](A)[sP].[dR](T)[sP].
[dR](T)[sP].[dR](A)[sP].[dR](T)[sP].[dR](A)[sP].[dR](G)[sP].[mR](G)[sP].[dR](T)[sP].
[dR](T)[sP].[dR](T)[sP].[LR](T)[sP].[LR](T)[sP].[LR]([5meC])[PS2].[LR](T),
(ay)
(SEQ ID NO 6, CMP ID NO 6_9, CNJ ID NO 6_9)
[5gn2c6]P.[dR](C)P.[dR](A)P.[MOE](G)[sP].[LR]([5meC])[PS2].[dR](A)[sP].[dR](T)
[sP].[dR](T)[sP].[dR](A)[sP].[dR](T)[sP].[dR](A)[sP].[dR](G)[sP].[dR](G)[sP].[dR](T)
[sP].[dR](T)[sP].[dR](T)[sP].[LR](T)[sP].[LR](T)[sP].[LR]([5meC])[PS2].[MOE](T),
(az)
(SEQ ID NO 6, CMP ID NO 6_10, CNJ ID NO 6_10)
[5gn2c6]P.[dR](C)P.[dR](A)P.[MOE](G)[sP].[LR]([5meC])[PS2].[dR](A)[sP].[dR](T)
[sP].[dR](T)[sP].[dR](A)[sP].[dR](T)[sP].[dR](A)[sP].[dR](G)[sP].[dR](G)[sP].[dR](T)
[sP].[dR](T)[sP].[dR](T)[sP].[LR](T)[sP].[LR](T)[PS2].[MOE]([5meC])[sP].[MOE](T),
(ba)
(SEQ ID NO 6, CMP ID NO 6_11, CNJ ID NO 6_11)
[5gn2c6]P.[dR](C)P.[dR](A)P.[MOE](G)[sP].[LR]([5meC])[PS2].[dR](A)[sP].[dR](T)
[sP].[dR](T)[sP].[dR](A)[sP].[dR](T)[sP].[dR](A)[P].[dR](G)[sP].[dR](G)[sP].[dR](T)
[sP].[dR](T)[sP].[dR](T)[sP].[LR](T)[sP].[LR](T)[PS2].[LR]([5meC])[PS2].[MOE](T),
(bb)
(SEQ ID NO 6, CMP ID NO 6_12, CNJ ID NO 6_12)
[5gn2c6]P.[dR](C)P.[dR](A)P.[MOE](G)[sP].[LR]([5meC])[PS2].[dR](A)[sP].[dR](T)
[sP].[dR](T)[sP].[dR](A)[sP].[dR](T)[P].[dR](A)[sP].[dR](G)[sP].[dR](G)[sP].[dR](T)
[sP].[dR](T)[sP].[dR](T)[sP].[LR](T)[PS2].[LR](T)[PS2].[MOE]([5meC])[sP].[MOE](T),
(bc)
(SEQ ID NO 21, CMP ID NO 21_1, CNJ ID NO 21_1)
[5gn2c6]P.[dR](C)P.[dR](A)P.[LR](G)[PS2].[LR]([5meC])[sP].[dR](A)[P].[dR](T)[sP].
[dR](T)[sP].[dR](A)[sP].[dR](T)[sP].[dR](A)[sP].[dR](G)[sP].[dR](G)[sP].[dR](T)[sP].
[LR](T)[sP].[dR](T)[sP].[dR](T)[sP].[mR](U)[sP].[dR](C)[sP].[LR](T)[PS2].[LR]([5meC]),
(bd)
(SEQ ID NO 22, CMP ID NO 22_1, CNJ ID NO 22_1)
[5gn2c6]P.[dR](C)P.[dR](A)P.[LR](G)[PS2].[LR]([5meC])[sP].[dR](A)[sP].[dR](T)[sP].
[dR](T)[sP].[dR](A)[sP].[dR](T)[sP].[dR](A)[sP].[dR](G)[sP].[dR](G)[sP].[dR](T)[sP].
[LR](T)[sP].[dR](T)[sP].[mR](U)[sP].[dR](T)[sP].[dR](C)[sP].[LR](T)[PS2].[LR]([5meC]),
(be)
(SEQ ID NO 23, CMP ID NO 23_1, CNJ ID NO 23_1)
[5gn2c6]P.[dR](C)P.[dR](A)P.[LR](G)[PS2].[LR]([5meC])[sP].[dR](A)[sP].[dR](T)[sP].
[dR](T)[sP].[dR](A)[sP].[dR](T)[sP].[dR](A)[sP].[dR](G)[sP].[dR](G)[sP].[dR](T)[sP].
[LR](T)[sP].[mR](U)[sP].[dR](T)[sP].[dR](T)[sP].[dR](C)[sP].[LR](T)[PS2].[LR]([5meC]),
(bf)
(SEQ ID NO 24, CMP ID NO 24_1, CNJ ID NO 24_1)
[5gn2c6]P.[dR](C)P.[dR](A)P.[LR](G)[PS2].[LR]([5meC])[sP].[dR](A)[sP].[dR](T)[sP].
[dR](T)[sP].[dR](A)[sP].[dR](T)[sP].[dR](A)[sP].[dR](G)[sP].[dR](G)[sP].[dR](T)[sP].
[dR](T)[sP].[mR](U)[sP].[LR](T)[sP].[LR](T)[sP].[LR]([5meC])[PS2].[LR](T),
(bg)
(SEQ ID NO 25, CMP ID NO 25_1, CNJ ID NO 25_1)
[5gn2c6]P.[dR](C)P.[dR](A)P.[LR](G)[PS2].[LR]([5meC])[sP].[LR](A)[sP].[dR](T)[sP].
[dR](T)[sP].[dR](A)[sP].[dR](T)[sP].[dR](A)[sP].[dR](G)[sP].[dR](G)[sP].[dR](T)[sP].
[mR](U)[sP].[dR](T)[sP].[LR](T)[sP].[dR](T)[sP].[LR]([5meC])[PS2].[LR](T),
(bh)
(SEQ ID NO 25, CMP ID NO 25_2, CNJ ID NO 25_2)
[5gn2c6]P.[dR](C)P.[dR](A)P.[LR](G)[PS2].[LR]([5meC])[sP].[dR](A)[sP].[dR](T)[sP].
[dR](T)[sP].[dR](A)[sP].[dR](T)[sP].[dR](A)[sP].[dR](G)[sP].[dR](G)[sP].[dR](T)[sP].
[mR](U)[sP].[dR](T)[sP].[LR](T)[sP].[LR](T)[sP].[LR]([5meC])[PS2].[LR](T),
(bi)
(SEQ ID NO 26, CMP ID NO 26_1, CNJ ID NO 26_1)
[5gn2c6]P.[dR](C)P.[dR](A)P.[LR](G)[PS2].[LR]([5meC])[sP].[dR](A)[sP].[dR](T)[sP].
[dR](T)[sP].[dR](A)[sP].[dR](T)[sP].[dR](A)[sP].[dR](G)[sP].[dR](G)[sP].[mR](U)[sP].
[LR](T)[sP].[dR](T)[sP].[dR](T)[sP].[dR](T)[sP].[dR](C)[sP].[LR](T)[PS2].[LR]([5meC]),
(bj)
(SEQ ID NO 27, CMP ID NO 27_1, CNJ ID NO 27_1)
[5gn2c6]P.[dR](C)P.[dR](A)P.[LR](G)[PS2].[LR]([5meC])[sP].[LR](A)[sP].[dR](T)[sP].
[dR](T)[sP].[dR](A)[sP].[dR](T)[sP].[dR](A)[sP].[dR](G)[sP].[dR](G)[sP].[mR](U)[sP].
[dR](T)[sP].[dR](T)[sP].[LR](T)[sP].[dR](T)[sP].[LR]([5meC])[PS2].[LR](T),
(bk)
(SEQ ID NO 27, CMP ID NO 27_2, CNJ ID NO 27_2)
[5gn2c6]P.[dR](C)P.[dR](A)P.[LR](G)[PS2].[LR]([5meC])[sP].[dR](A)[P].[dR](T)[sP].
[dR](T)[sP].[dR](A)[sP].[dR](T)[sP].[dR](A)[sP].[dR](G)[sP].[dR](G)[sP].[mR](U)[sP].
[dR](T)[sP].[dR](T)[sP].[LR](T)[sP].[LR](T)[sP].[LR]([5meC])[PS2].[LR](T),
(bl)
(SEQ ID NO 28, CMP ID NO 28_1, CNJ ID NO 28_1)
[5gn2c6]P.[dR](C)P.[dR](A)P.[LR](G)[PS2].[LR]([5meC])[sP].[dR](A)[sP].[dR](T)[sP].
[dR](T)[sP].[dR](A)[sP].[mR](U)[sP].[dR](A)[sP].[dR](G)[sP].[dR](G)[sP].[dR](T)[sP].
[dR](T)[sP].[dR](T)[sP].[LR](T)[sP].[LR](T)[sP].[LR]([5meC])[PS2].[LR](T),
(bm)
(SEQ ID NO 29, CMP ID NO 29_1, CNJ ID NO 29_1)
[5gn2c6]P.[dR](C)P.[dR](A)P.[LR](G)[PS2].[LR]([5meC])[sP].[dR](A)[sP].[dR](T)[sP].
[mR](U)[sP].[dR](A)[sP].[dR](T)[sP].[dR](A)[sP].[dR](G)[sP].[dR](G)[sP].[dR](T)[sP].
[dR](T)[sP].[dR](T)[sP].[LR](T)[sP].[LR](T)[sP].[LR]([5meC])[PS2].[LR](T),
(bn)
(SEQ ID NO 30, CMP ID NO 30_1, CNJ ID NO 30_1)
[5gn2c6]P.[dR](C)P.[dR](A)P.[LR](G)[PS2].[LR]([5meC])[sP].[dR](A)[sP].[mR](U)[sP].
[dR](T)[sP].[dR](A)[sP].[dR](T)[sP].[dR](A)[sP].[dR](G)[sP].[dR](G)[sP].[dR](T)[sP].
[dR](T)[sP].[dR](T)[sP].[LR](T)[sP].[LR](T)[sP].[LR]([5meC])[PS2].[LR](T),
(bo)
(SEQ ID NO 11, CMP ID NO 11_2, CNJ ID NO 11_2)
[5gn2c6]P.[dR](C)P.[dR](A)P.[LR](G)[P].[LR](T)[sP].[LR](T)[sP].[dR](T)[sP].[dR](T)
[sP].[dR](A)[sP].[dR](A)[sP].[mR](G)[sP].[dR](A)[sP].[dR](T)[sP].[dR](T)[sP].[dR](G)
[sP].[dR](T)[sP].[dR](G)[sP].[dR](G)[sP].[dR](T)[sP].[dR](G)[sP].[LR](T)[sP].[LR](T)
[sP].[LR](T),
and
(bp)
(SEQ ID NO 11, CMP ID NO 11_3, CNJ ID NO 11_3)
[5gn2c6]P.[dR](C)P.[dR](A)P.[LR](G)[sP].[LR](T)[sP].[LR](T)[sP].[dR](T)[sP].[dR](T)
[sP].[dR](A)[sP].[dR](A)[sP].[mR](G)[sP].[dR](A)[sP].[dR](T)[sP].[dR](T)[sP].[dR](G)
[sP].[dR](T)[sP].[dR](G)[sP].[dR](G)[sP].[dR](T)[sP].[dR](G)[sP].[LR](T)[sP].[LR](T)
[PS2].[LR](T),
wherein
(A) is a nucleoside comprising an adenine nucleobase,
(C) is a nucleoside comprising a cytosine nucleobase,
([5meC]) is a nucleoside comprising a 5-methyl cytosine nucleobase,
([PPG]) is a nucleoside comprising a 7-deaza-8-aza guanine nucleobase,
(G) is a nucleoside comprising a guanine nucleobase,
(U) is a nucleoside comprising a uracil nucleobase,
(T) is a nucleoside comprising a thymine nucleobase,
[LR] indicates that the following nucleoside is a beta-D-oxy-LNA nucleoside,
[dR] indicates that the following nucleoside is a DNA nucleoside,
[mR] indicates that the following nucleoside is a 2′-O-methyl RNA nucleoside,
[MOE] indicates that the following nucleoside is a 2′-O-methoxyethyl RNA (MOE RNA) nucleoside,
[sP] indicates a phosphorothioate internucleoside linkage between the preceding and following nucleoside,
[PS2] indicates a phosphorodithioate internucleoside linkage between the preceding and following nucleoside,
P indicates a phosphodiester internucleoside linkage between the preceding nucleoside or moiety and the following nucleoside, and
[5gn2c6] is an aminohexyl conjugated tri(N-acetyl-galactosamine) moiety as shown in FIG. 8.

26. (canceled)

27. The antisense oligonucleotide or the antisense oligonucleotide conjugate of claim 1, wherein the target sequence is a sequence of at least 17 contiguous nucleotides within the sequence GCCUAUCUGAGAAACUUUU (SEQ ID NO 32) (positions 2181-2199 of SEQ ID NO 45).

28. The antisense oligonucleotide or the antisense oligonucleotide conjugate of claim 27, wherein the target sequence is GCCUAUCUGAGAAACUUUU (SEQ ID NO 32) (positions 2181-2199 of SEQ ID NO 45).

29. The antisense oligonucleotide or the antisense oligonucleotide conjugate of claim 27, wherein the sequence of the antisense oligonucleotide and/or the sequence of the contiguous nucleotide sequence comprises or consists of a sequence having at least 80%, at least 85%, at least 90%, at least 95% or 100% identity to AAAAGTTTCTCAGATAGGE (SEQ ID NO 2).

30. An antisense oligonucleotide comprising or consisting of the following compound:
(SEQ ID NO 2, CMP ID NO 2_1)
[LR](A)[PS2].[LR](A)[sP].[dR](A)[sP].[mR](A)[sP].[LR](G)[sP].[dR](T)[sP].[dR](T)[sP].
[dR](T)[sP].[dR](C)[sP].[dR](T)[sP].[dR](C)[sP].[dR](A)[sP].[dR](G)[sP].[dR](A)[sP].
[dR](T)[sP].[dR](A)[sP].[mR](G)[sP].[LR](G)[PS2].[LR]([5meC]),
wherein
(A) is a nucleoside comprising an adenine nucleobase,
(C) is a nucleoside comprising a cytosine nucleobase,
([5meC]) is a nucleoside comprising a 5-methyl cytosine nucleobase,
([PPG]) is a nucleoside comprising a 7-deaza-8-aza guanine nucleobase,
(G) is a nucleoside comprising a guanine nucleobase,
(U) is a nucleoside comprising a uracil nucleobase,
(T) is a nucleoside comprising a thymine nucleobase,
[LR] indicates that the following nucleoside is a beta-D-oxy-LNA nucleoside,
[dR] indicates that the following nucleoside is a DNA nucleoside,
[mR] indicates that the following nucleoside is a 2′-O-methyl RNA nucleoside,
[MOE] indicates that the following nucleoside is a 2′-O-methoxyethyl RNA (MOE RNA) nucleoside,
[sP] indicates a phosphorothioate internucleoside linkage between the preceding and following nucleoside, and
[PS2] indicates a phosphorodithioate internucleoside linkage between the preceding and following nucleoside.

31. An antisense oligonucleotide conjugate of claim 25, wherein the antisense oligonucleotide conjugate is the following:
(SEQ ID NO 2, CMP ID NO 2_1, CNJ ID NO 2_1)
[5gn2c6]P.[dR](C)P.[dR](A)P.[LR](A)[PS2].[LR](A)[sP].[dR](A)[sP].[mR](A)[sP].[LR]
(G)[sP].[dR](T)[sP].[dR](T)[sP].[dR](T)[sP].[dR](C)[sP].[dR](T)[sP].[dR](C)[sP].[dR](A)
[sP].[dR](G)[sP].[dR](A)[sP].[dR](T)[sP].[dR](A)[sP].[mR](G)[sP].[LR](G)[PS2].[LR]
([5meC]),
wherein
(A) is a nucleoside comprising an adenine nucleobase,
(C) is a nucleoside comprising a cytosine nucleobase,
([5meC]) is a nucleoside comprising a 5-methyl cytosine nucleobase,
([PPG]) is a nucleoside comprising a 7-deaza-8-aza guanine nucleobase,
(G) is a nucleoside comprising a guanine nucleobase,
(U) is a nucleoside comprising a uracil nucleobase,
(T) is a nucleoside comprising a thymine nucleobase,
[LR] indicates that the following nucleoside is a beta-D-oxy-LNA nucleoside,
[dR] indicates that the following nucleoside is a DNA nucleoside,
[mR] indicates that the following nucleoside is a 2′-O-methyl RNA nucleoside,
[MOE] indicates that the following nucleoside is a 2′-O-methoxyethyl RNA (MOE RNA) nucleoside,
[sP] indicates a phosphorothioate internucleoside linkage between the preceding and following nucleoside,
[PS2] indicates a phosphorodithioate internucleoside linkage between the preceding and following nucleoside,
P indicates a phosphodiester internucleoside linkage between the preceding nucleoside or moiety and the following nucleoside, and
[5gn2c6] is an aminohexyl conjugated tri(N-acetyl-galactosamine) moiety as shown in FIG. 8.

32. (canceled)

33. The antisense oligonucleotide or antisense oligonucleotide conjugate of claim 1, wherein the target sequence is a sequence of at least 17 contiguous nucleotides within AAGUAAAAUUAACAUGUCCA (SEQ ID NO 43) (positions 16970-16989 of SEQ ID NO 45).

34. The antisense oligonucleotide or antisense oligonucleotide conjugate of claim 33, wherein the target sequence is AAGUAAAAUUAACAUGUCC (SEQ ID NO 33) (positions 16970-16988 of SEQ ID NO 45).

35. The antisense oligonucleotide or antisense oligonucleotide conjugate of claim 33, wherein the sequence of the antisense oligonucleotide and/or the sequence of the contiguous nucleotide sequence comprises or consists of a sequence having at least 80%, at least 85%, at least 90%, at least 95% or 100% identity to GGACATGTTAATTTTACTT (SEQ ID NO 3).

36. An antisense oligonucleotide comprising or consisting of the following compound:
(SEQ ID NO 3, CMP ID NO 3_1)
[LR](G)[sP].[LR](G)[sP].[mR](A)[sP].[dR](C)[sP].[dR](A)[sP].[dR](T)[sP].[LR](G)[sP].
[dR](T)[sP].[dR](T)[sP].[dR](A)[sP].[dR](A)[sP].[dR](T)[sP].[dR](T)[sP].[dR](T)[sP].
[dR](T)[sP].[dR](A)[sP].[dR](C)[sP].[LR](T)[PS2].[LR](T),
wherein
(A) is a nucleoside comprising an adenine nucleobase,
(C) is a nucleoside comprising a cytosine nucleobase,
([5meC]) is a nucleoside comprising a 5-methyl cytosine nucleobase,
([PPG]) is a nucleoside comprising a 7-deaza-8-aza guanine nucleobase,
(G) is a nucleoside comprising a guanine nucleobase,
(U) is a nucleoside comprising a uracil nucleobase,
(T) is a nucleoside comprising a thymine nucleobase,
[LR] indicates that the following nucleoside is a beta-D-oxy-LNA nucleoside,
[dR] indicates that the following nucleoside is a DNA nucleoside,
[mR] indicates that the following nucleoside is a 2′-O-methyl RNA nucleoside,
[MOE] indicates that the following nucleoside is a 2′-O-methoxyethyl RNA (MOE RNA) nucleoside,
[sP] indicates a phosphorothioate internucleoside linkage between the preceding and following nucleoside, and
[PS2] indicates a phosphorodithioate internucleoside linkage between the preceding and following nucleoside.

37. An antisense oligonucleotide conjugate of claim 25, wherein the antisense oligonucleotide conjugate is the following:
(SEQ ID NO 3, CMP ID NO 3_1, CNJ ID NO 3_1)
[5gn2c6]P.[dR](C)P.[dR](A)P.[LR](G)[sP].[LR](G)[sP].[mR](A)[sP].[dR](C)[sP].[dR]
(A)[sP].[dR](T)[sP].[LR](G)[sP].[dR](T)[sP].[dR](T)[sP].[dR](A)[sP].[dR](A)[sP].[dR](T)
[sP].[dR](T)[sP].[dR](T)[sP].[dR](T)[sP].[dR](A)[sP].[dR](C)[sP].[LR](T)[PS2].[LR](T),
wherein
(A) is a nucleoside comprising an adenine nucleobase,
(C) is a nucleoside comprising a cytosine nucleobase,
([5meC]) is a nucleoside comprising a 5-methyl cytosine nucleobase,
([PPG]) is a nucleoside comprising a 7-deaza-8-aza guanine nucleobase,
(G) is a nucleoside comprising a guanine nucleobase,
(U) is a nucleoside comprising a uracil nucleobase,
(T) is a nucleoside comprising a thymine nucleobase,
[LR] indicates that the following nucleoside is a beta-D-oxy-LNA nucleoside,
[dR] indicates that the following nucleoside is a DNA nucleoside,
[mR] indicates that the following nucleoside is a 2′-O-methyl RNA nucleoside,
[MOE] indicates that the following nucleoside is a 2′-O-methoxyethyl RNA (MOE RNA) nucleoside,
[sP] indicates a phosphorothioate internucleoside linkage between the preceding and following nucleoside,
[PS2] indicates a phosphorodithioate internucleoside linkage between the preceding and following nucleoside,
P indicates a phosphodiester internucleoside linkage between the preceding nucleoside or moiety and the following nucleoside, and
[5gn2c6] is an aminohexyl conjugated tri(N-acetyl-galactosamine) moiety as shown in FIG. 8.

38. (canceled)

39. The antisense oligonucleotide or the antisense oligonucleotide conjugate of claim 1, wherein the antisense oligonucleotide or the antisense oligonucleotide conjugate is in the form of a pharmaceutically acceptable salt.

40. The antisense oligonucleotide or the antisense oligonucleotide conjugate of claim 1, wherein the antisense oligonucleotide or the antisense oligonucleotide conjugate is encapsulated in a lipid-based delivery vehicle, covalently linked to or encapsulated in a dendrimer, or conjugated to an aptamer.

41. A pharmaceutical composition comprising the antisense oligonucleotide or the antisense oligonucleotide conjugate of claim 1, and a pharmaceutically acceptable diluent, solvent, carrier, salt and/or adjuvant.

42. An in vitro or in vivo method for reducing A1CF expression in a target cell, the method comprising administering an effective amount of the antisense oligonucleotide or the antisense oligonucleotide conjugate of claim 1.

43. A method for treating or preventing a disease comprising administering a therapeutically or prophylactically effective amount of the antisense oligonucleotide or the antisense oligonucleotide conjugate of claim 1.

44-45. (canceled)

46. The method of claim 43, wherein the disease is HBV infection.

47. An in vitro or in vivo method for reducing A1CF expression in a target cell, the method comprising administering an effective amount of the pharmaceutical composition of claim 41 to the target cell.

48. A method for treating or preventing a disease comprising administering a therapeutically or prophylactically effective amount of the pharmaceutical composition of claim 41 to a subject suffering from or susceptible to a disease.
